(19) United States
(12) Patent Application Publication
(10) Pub. No.: US 2026/0285875 A1
(43) Pub. Date: Sep. 24, 2026

(54) INHIBITORS OF PARG

(71) Applicant: 858 Therapeutics, Inc., San Diego, CA (US)

(72) Inventors: Shyama HERATH, San Diego, CA (US); James M. VEAL, Solana Beach, CA (US); Jeffrey A. STAFFORD, Rancho Santa Fe, CA (US); Taylor ALEXANDER, Carlsbad, CA (US); Grant SEILER, La Jolla, CA (US); Riley MILLS, Del Mar, CA (US); Christopher MCBRIDE, San Diego, CA (US); Donald S. KARANEWSKY, Escondido, CA (US)

(21) Appl. No.: 19/671,000

(22) Filed: May 7, 2026

Related U.S. Application Data
(63) Continuation of application No. PCT/US2024/056940, filed on Nov. 21, 2024.
(60) Provisional application No. 63/602,264, filed on Nov. 22, 2023.

(51) Int. Cl. C07D 498/04; A61K 31/433; A61K 31/519; A61K 31/5365; A61K 31/537; A61K 31/5377; A61K 31/5395; C07B 59/00; C07D 471/14; C07D 487/04; C07D 487/10; C07D 487/14; C07D 491/147; C07D 495/14; C07D 519/00

(52) U.S. Cl. CPC C07D 498/04; A61K 31/433; A61K 31/519; A61K 31/5365; A61K 31/537; A61K 31/5377; A61K 31/5395; C07B 59/002; C07D 471/14; C07D 487/04; C07D 487/10; C07D 487/14; C07D 491/147; C07D 495/14; C07D 519/00; C07B 2200/05

(57) ABSTRACT

Provided herein are inhibitors of PARG, pharmaceutical compositions comprising the inhibitory compounds, and methods for using the PARG inhibitory compounds for the treatment of disease.

### CROSS-REFERENCE TO RELATED APPLICATION

This application is a continuation of International Application No. PCT/US2024/056940, Filed Nov. 21, 2024, which claims the benefit of U.S. Patent Application No. 63/602,264, filed on Nov. 22, 2023, which are hereby incorporated by reference in their entirety.

### BACKGROUND

Poly(ADP-ribose)glycohydrolase (PARG) is an enzyme that plays a critical role in DNA damage response. PARG inhibition or depletion has been shown to be beneficial for the treatment of certain cancer types, and PARG-depleted or inhibited cancer cells also show an increased sensitivity to other therapies such as DNA damaging agents, cell cycle checkpoint inhibitors, and inhibitors of enzymes involved in nucleotide metabolism. PARG inhibitors are anticipated to have utility as a cancer treatment both as single agents and in combination with therapeutic agents and radiotherapy.

### BRIEF SUMMARY OF THE INVENTION

Provided herein are inhibitors of PARG, pharmaceutical compositions comprising said inhibitory compounds, and methods for using said inhibitory compounds for the treatment of disease.

One embodiment provides a compound, or a pharmaceutically acceptable salt, stereoisomer, or deuteroisotope thereof, having the structure of Formula (I):

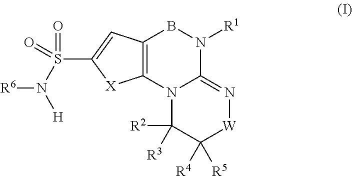

wherein,
B is a bond, C═O, or CN—OR⁹;
W is a bond, —O—, or C(R¹⁰)(R¹¹), or NR⁷;
X is —S—, or —*Y═C(A)—; wherein the * indicates point of attachment to C—SO₂ group;
Y is N, C—H, C—F, C—O(optionally substituted C1-C6 alkyl), C—NH₂, C—NH(optionally substituted C1-C6 alkyl), C—N(optionally substituted C1-C6 alkyl)₂, C—SH, C—S(optionally substituted C1-C6 alkyl), C—NH(optionally substituted (heteroaryl)alkylene), —C—NH(optionally substituted (heteroaryl)alkynylene);
A is selected from the group consisting of hydrogen, halo, —OH, —CN, optionally substituted C1-C6 alkoxy, —N(R⁷)₂, optionally substituted C1-C6 alkyl, optionally substituted C2-C6 alkenyl, optionally substituted C2-C6 alkynyl, optionally substituted C3-C7 carbocyclyl, optionally substituted (carbocyclyl)alkylene, optionally substituted (carbocyclyl)alkynylene, optionally substituted heterocyclyl, optionally substituted (heterocyclyl)alkylene, optionally substituted heteroaryl, and optionally substituted aryl;
R¹ is selected from the group consisting of optionally substituted alkyl, optionally substituted alkenyl, optionally substituted alkynyl, optionally substituted (cycloalkyl)alkylene, optionally substituted (aryl)alkylene, optionally substituted (heterocyclyl)alkylene, optionally substituted (heteroaryl)alkylene, optionally substituted (heteroaryl)alkynylene, optionally substituted aryl, optionally substituted heteroaryl, optionally substituted C3-C7 carbocyclyl, and optionally substituted heterocyclyl;
R² and R³ are independently selected from the group consisting of hydrogen, halo, —OH, —CN, optionally substituted alkyl, optionally substituted alkenyl, optionally substituted alkynyl, optionally substituted cycloalkyl, optionally substituted aryl, optionally substituted heteroaryl, optionally substituted heterocyclyl, optionally substituted (cycloalkyl)alkylene, optionally substituted (heterocyclyl)alkylene, optionally substituted (aryl)alkylene, optionally substituted (heteroaryl)alkylene, optionally substituted (cycloalkyl)alkenylene, optionally substituted (heterocyclyl)alkenylene, optionally substituted (aryl)alkenylene, optionally substituted (heteroaryl)alkenylene, optionally substituted (cycloalkyl)alkynylene, optionally substituted (heterocyclyl)alkynylene, optionally substituted (heteroaryl)alkynylene, optionally substituted (aryl)alkynylene, optionally substituted (cycloalkyl)-O-alkylene, optionally substituted (heterocyclyl)-O-alkylene, optionally substituted (aryl)-O-alkylene, optionally substituted (heteroaryl)-O-alkylene, optionally substituted (cycloalkyl)-NR⁷-alkylene, optionally substituted (heterocyclyl)-NR⁷-alkylene, optionally substituted (aryl)-NR⁷-alkylene, optionally substituted (heteroaryl)-NR⁷-alkylene, optionally substituted (alkyl)-O-alkylene, optionally substituted (alkyl)-NR⁷-alkylene, optionally substituted (alkyl)-O-alkynylene, and optionally substituted (alkyl)-NR⁷-alkynylene; or
optionally, R² and R³ combine to form an optionally substituted carbocyclic or heterocyclic ring;
R⁴, R⁵, R¹⁰, and R¹¹ are independently selected from the group consisting of hydrogen, halo, —OH, —CN, optionally substituted alkyl, optionally substituted alkenyl, optionally substituted alkynyl, optionally substituted cycloalkyl, optionally substituted (cycloalkyl)alkylene, optionally substituted (cycloalkyl)alkynylene, optionally substituted heterocyclyl, optionally substituted (heterocyclyl)alkylene, optionally substituted heteroaryl, and optionally substituted aryl;
or optionally, R⁴ and R⁵ combine to form an optionally substituted carbocyclic or heterocyclic ring;
or optionally, R³ and R⁴ combine to form an optionally substituted carbocyclic or heterocyclic ring;
R⁶ is optionally substituted alkyl, optionally substituted cycloalkyl, optionally substituted heterocyclyl, or an optionally substituted 1,1′-bi(cyclopropan)-1-yl;
each R⁷ is independently hydrogen or an optionally substituted C1-C6 alkyl; and
each R⁹ is independently hydrogen or an optionally substituted C1-C6 alkyl.

One embodiment provides a compound, or a pharmaceutically acceptable salt, stereoisomer, or deuteroisotope thereof, having the structure of Formula (Ia):

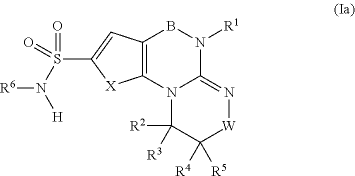

wherein,
B is a bond, C═O, or CN—OR⁹;
W is a bond, —O—, or C(R¹⁰)(R¹¹), or NR⁷;
X is —S—, or —Y═C(A)—;
Y is N, C—H, or C—F;
A is selected from the group consisting of hydrogen, halo, —OH, —CN, optionally substituted C1-C6 alkoxy, —N(R⁷)₂, optionally substituted C1-C6 alkyl, optionally substituted C2-C6 alkenyl, optionally substituted C2-C6 alkynyl, optionally substituted C3-C7 carbocyclyl, optionally substituted (carbocyclyl)alkylene, optionally substituted (carbocyclyl)alkynylene, optionally substituted heterocyclyl, optionally substituted (heterocyclyl)alkylene, optionally substituted heteroaryl, and optionally substituted aryl;
R¹ is selected from the group consisting of optionally substituted alkyl, optionally substituted alkenyl, optionally substituted alkynyl, optionally substituted (cycloalkyl)alkylene, optionally substituted (aryl)alkylene, optionally substituted (heterocyclyl)alkylene, optionally substituted (heteroaryl)alkylene, optionally substituted aryl, optionally substituted heteroaryl, optionally substituted C3-C7 carbocyclyl, and optionally substituted heterocyclyl;
R² and R³ are independently selected from the group consisting of hydrogen, halo, —OH, —CN, optionally substituted alkyl, optionally substituted alkenyl, optionally substituted alkynyl, optionally substituted cycloalkyl, optionally substituted aryl, optionally substituted heteroaryl, optionally substituted heterocyclyl, optionally substituted (cycloalkyl)alkylene, optionally substituted (heterocyclyl)alkylene, optionally substituted (aryl)alkylene, optionally substituted (heteroaryl)alkylene, optionally substituted (cycloalkyl)alkenylene, optionally substituted (heterocyclyl)alkenylene, optionally substituted (aryl)alkenylene, optionally substituted (heteroaryl)alkenylene, optionally substituted (cycloalkyl)alkynylene, optionally substituted (heterocyclyl)alkynylene, optionally substituted (heteroaryl)alkynylene, optionally substituted (aryl)alkynylene, optionally substituted (cycloalkyl)-O-alkylene, optionally substituted (heterocyclyl)-O-alkylene, optionally substituted (aryl)-O-alkylene, optionally substituted (heteroaryl)-O-alkylene, optionally substituted (cycloalkyl)-NR⁷-alkylene, optionally substituted (heterocyclyl)-NR⁷-alkylene, optionally substituted (aryl)-NR⁷-alkylene, optionally substituted (heteroaryl)-NR⁷-alkylene, optionally substituted (alkyl)-O-alkylene, optionally substituted (alkyl)-NR⁷-alkylene, optionally substituted (alkyl)-O-alkynylene, and optionally substituted (alkyl)-NR⁷-alkynylene; or
optionally, R² and R³ combine to form an optionally substituted carbocyclic or heterocyclic ring;
R⁴, R⁵, R¹⁰, and R¹¹ are independently selected from the group consisting of hydrogen, halo, —OH, —CN, optionally substituted alkyl, optionally substituted alkenyl, optionally substituted alkynyl, optionally substituted cycloalkyl, optionally substituted (cycloalkyl)alkylene, optionally substituted (cycloalkyl)alkynylene, optionally substituted heterocyclyl, optionally substituted (heterocyclyl)alkylene, optionally substituted heteroaryl, and optionally substituted aryl;
or optionally, R⁴ and R⁵ combine to form an optionally substituted carbocyclic or heterocyclic ring;
or optionally, R³ and R⁴ combine to form an optionally substituted carbocyclic or heterocyclic ring;
R⁶ is optionally substituted alkyl, optionally substituted cycloalkyl, optionally substituted heterocyclyl, or an optionally substituted 1,1′-bi(cyclopropan)-1-yl;
each R⁷ is independently hydrogen or an optionally substituted C1-C6 alkyl; and
each R⁹ is independently hydrogen or an optionally substituted C1-C6 alkyl.

One embodiment provides a pharmaceutical composition comprising a compound of Formula (I), or a pharmaceutically acceptable salt or solvate thereof, and at least one pharmaceutically acceptable excipient.

One embodiment provides a method of treating a disease or disorder in a patient in need thereof comprising administering to the patient a compound of Formula (I), or a pharmaceutically acceptable salt or solvate thereof. Another embodiment provides the method wherein the disease or disorder is cancer.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.
“Amino” refers to the —NH₂ radical.
“Cyano” refers to the —CN radical.
“Nitro” refers to the —NO₂ radical.
“Oxa” refers to the —O— radical.
“Oxo” refers to the ═O radical.
“Thioxo” refers to the ═S radical.
“Imino” refers to the ═N—H radical.
“Oximo” refers to the ═N—OH radical.
“Hydrazino” refers to the ═N—NH₂ radical.
“Alkyl” refers to a straight or branched hydrocarbon chain radical consisting solely of carbon and hydrogen atoms, containing no unsaturation, having from one to fifteen carbon atoms (e.g., C1-C15 alkyl). In certain embodiments, an alkyl comprises one to thirteen carbon atoms (e.g., C1-C13 alkyl). In certain embodiments, an alkyl comprises one to eight carbon atoms (e.g., C1-C8 alkyl). In other embodiments, an alkyl comprises one to five carbon atoms (e.g., C1-C5 alkyl). In other embodiments, an alkyl comprises one to four carbon atoms (e.g., C1-C4 alkyl). In other embodiments, an alkyl comprises one to three carbon atoms (e.g., C1-C3 alkyl). In other embodiments, an alkyl comprises one to two carbon atoms (e.g., C1-C2 alkyl). In other embodiments, an alkyl comprises one carbon atom (e.g., C1 alkyl). In other embodiments, an alkyl comprises five to fifteen carbon atoms (e.g., C5-C15 alkyl). In other embodiments, an alkyl comprises five to eight carbon atoms (e.g., C5-C8 alkyl). In other embodiments, an alkyl comprises two to five carbon atoms (e.g., C2-C5 alkyl). In other embodiments, an alkyl comprises three to five carbon atoms (e.g., C3-C5 alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —ORa, —SRa, —OC(O)—Ra, —N(Ra)₂, —C(O)Ra, —C(O)ORa, —C(O)N(Ra)₂, —N(Ra)C(O)ORa, —OC(O)—N(Ra)₂, —N(Ra)C(O)Ra, —N(Ra)S(O)tRa (where t is 1 or 2), —S(O)tORa (where t is 1 or 2), —S(O)tRa (where t is 1 or 2) and —S(O)tN(Ra)2 (where t is 1 or 2) where each Ra is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl). In certain embodiments, an optionally substituted alkyl is a haloalkyl. In other embodiments, an optionally substituted alkyl is a fluoroalkyl. In other embodiments, an optionally substituted alkyl is a —CF3 group.
“Alkoxy” refers to a radical bonded through an oxygen atom of the formula —O-alkyl, where alkyl is an alkyl chain as defined above.
“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —ORa, —SRa, —OC(O)—Ra, —N(Ra)₂, —C(O)Ra, —C(O)ORa, —C(O)N(Ra)₂, —N(Ra)C(O)ORa, —OC(O)—N(Ra)₂, —N(Ra)C(O)Ra, —N(Ra)S(O)tRa (where t is 1 or 2), —S(O)tORa (where t is 1 or 2), —S(O)tRa (where t is 1 or 2) and —S(O)tN(Ra)2 (where t is 1 or 2) where each Ra is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).
“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —ORa, —SRa, —OC(O)—Ra, —N(Ra)₂, —C(O)Ra, —C(O)ORa, —C(O)N(Ra)₂, —N(Ra)C(O)ORa, —OC(O)—N(Ra)₂, —N(Ra)C(O)Ra, —N(Ra)S(O)tRa (where t is 1 or 2), —S(O)tORa (where t is 1 or 2), —S(O)tRa (where t is 1 or 2) and —S(O)tN(Ra)2 (where t is 1 or 2) where each Ra is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).
“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C1-C8 alkylene). In other embodiments, an alkylene comprises one to five carbon atoms (e.g., C1-C5 alkylene). In other embodiments, an alkylene comprises one to four carbon atoms (e.g., C1-C4 alkylene). In other embodiments, an alkylene comprises one to three carbon atoms (e.g., C1-C3 alkylene). In other embodiments, an alkylene comprises one to two carbon atoms (e.g., C1-C2 alkylene). In other embodiments, an alkylene comprises one carbon atom (e.g., C1 alkylene). In other embodiments, an alkylene comprises five to eight carbon atoms (e.g., C5-C8 alkylene). In other embodiments, an alkylene comprises two to five carbon atoms (e.g., C2-C5 alkylene). In other embodiments, an alkylene comprises three to five carbon atoms (e.g., C3-C5 alkylene). Unless stated otherwise specifically in the specification, an alkylene chain is optionally substituted by one or more of the following substituents: halo, cyano, nitro, oxo, thioxo, imino, oximo, trimethylsilanyl, —ORa, —SRa, —OC(O)—Ra, —N(Ra)₂, —C(O)Ra, —C(O)ORa, —C(O)N(Ra)₂, —N(Ra)C(O)ORa, —OC(O)—N(Ra)₂, —N(Ra)C(O)Ra, —N(Ra)S(O)tRa (where t is 1 or 2), —S(O)tORa (where t is 1 or 2), —S(O)tRa (where t is 1 or 2) and —S(O)tN(Ra)2 (where t is 1 or 2) where each Ra is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, amino, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).
“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C2-C8 alkenylene). In other embodiments, an alkenylene comprises two to five carbon atoms (e.g., C2-C5 alkenylene). In other embodiments, an alkenylene comprises two to four carbon atoms (e.g., C2-C4 alkenylene). In other embodiments, an alkenylene comprises two to three carbon atoms (e.g., C2-C3 alkenylene). In other embodiments, an alkenylene comprises two carbon atoms (e.g., C2 alkenylene). In other embodiments, an alkenylene comprises five to eight carbon atoms (e.g., C5-C8 alkenylene). In other embodiments, an alkenylene comprises three to five carbon atoms (e.g., C3-C5 alkenylene). Unless stated otherwise specifically in the specification, an alkenylene chain is optionally substituted by one or more of the following substituents: halo, cyano, nitro, oxo, thioxo, imino, oximo, trimethylsilanyl, —ORa, —SRa, —OC(O)—Ra, —N(Ra)₂, —C(O)Ra, —C(O)ORa, —C(O)N(Ra)₂, —N(Ra)C(O)ORa, —OC(O)—N(Ra)₂, —N(Ra)C(O)Ra, —N(Ra)S(O)tRa (where t is 1 or 2), —S(O)tORa (where t is 1 or 2), —S(O)tRa (where t is 1 or 2) and —S(O)tN(Ra)2 (where t is 1 or 2) where each Ra is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).
“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C2-C8 alkynylene). In other embodiments, an alkynylene comprises two to five carbon atoms (e.g., C2-C5 alkynylene). In other embodiments, an alkynylene comprises two to four carbon atoms (e.g., C2-C4 alkynylene). In other embodiments, an alkynylene comprises two to three carbon atoms (e.g., C2-C3 alkynylene). In other embodiments, an alkynylene comprises two carbon atoms (e.g., C2 alkynylene). In other embodiments, an alkynylene comprises five to eight carbon atoms (e.g., C5-C8 alkynylene). In other embodiments, an alkynylene comprises three to five carbon atoms (e.g., C3-C5 alkynylene). Unless stated otherwise specifically in the specification, an alkynylene chain is optionally substituted by one or more of the following substituents: halo, cyano, nitro, oxo, thioxo, imino, oximo, trimethylsilanyl, —ORa, —SRa, —OC(O)—Ra, —N(Ra)₂, —C(O)Ra, —C(O)ORa, —C(O)N(Ra)₂, —N(Ra)C(O)ORa, —OC(O)—N(Ra)₂, —N(Ra)C(O)Ra, —N(Ra)S(O)tRa (where t is 1 or 2), —S(O)tORa (where t is 1 or 2), —S(O)tRa (where t is 1 or 2) and —S(O)tN(Ra)2 (where t is 1 or 2) where each Ra is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).
“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from optionally substituted alkyl, optionally substituted alkenyl, optionally substituted alkynyl, halo, cyano, nitro, —Rb—ORa, —Rb—OC(O)—Ra, —Rb—OC(O)—ORa, —Rb—OC(O)—N(Ra)₂, —Rb—N(Ra)₂, —Rb—C(O)Ra, —Rb—C(O)ORa, —Rb—C(O)N(Ra)₂, —Rb—O—Re—C(O)N(Ra)₂, —Rb—N(Ra)C(O)ORa, —Rb—N(Ra)C(O)Ra, —Rb—N(Ra)S(O)tRa (where t is 1 or 2), —Rb—S(O)tRa (where t is 1 or 2), —Rb—S(O)tORa (where t is 1 or 2) and —Rb—S(O)tN(Ra)2 (where t is 1 or 2), where each Ra is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each Rb is independently a direct bond or a straight or branched alkylene or alkenylene chain, and Rc is a straight or branched alkylene or alkenylene chain, and where each of the Ra, Rb, or Rc substituents is unsubstituted unless otherwise indicated.
“Aralkyl” refers to a radical of the formula -Rc-aryl where Rc is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.
“Aralkenyl” refers to a radical of the formula -Rd-aryl where Rd is an alkenylene chain as defined above. The aryl part of the aralkenyl radical is optionally substituted as described above for an aryl group. The alkenylene chain part of the aralkenyl radical is optionally substituted as defined above for an alkenylene group.
“Aralkynyl” refers to a radical of the formula —Re-aryl, where Re is an alkynylene chain as defined above. The aryl part of the aralkynyl radical is optionally substituted as described above for an aryl group. The alkynylene chain part of the aralkynyl radical is optionally substituted as defined above for an alkynylene chain.
“Aralkoxy” refers to a radical bonded through an oxygen atom of the formula -O-Rc-aryl where Rc is an alkylene chain as defined above, for example, methylene, ethylene, and the like.
The alkylene chain part of the aralkyl radical is optionally substituted as described above for an alkylene chain. The aryl part of the aralkyl radical is optionally substituted as described above for an aryl group.
“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond.
Carbocyclyl is saturated (i.e., containing single C-C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from optionally substituted alkyl, optionally substituted alkenyl, optionally substituted alkynyl, halo, oxo, thioxo, cyano, nitro, —Rb—ORa, —Rb—OC(O)—Ra, —Rb—OC(O)—ORa, —Rb—OC(O)—N(Ra)₂, —Rb—N(Ra)₂, —Rb—C(O)Ra, —Rb—C(O)ORa, —Rb—C(O)N(Ra)₂, —Rb—O-Rc-C(O)N(Ra)₂, —Rb—N(Ra)C(O)ORa, —Rb—N(Ra)C(O)Ra, —Rb—N(Ra)S(O)tRa (where t is 1 or 2), —Rb—S(O)tRa (where t is 1 or 2), —Rb—S(O)tORa (where t is 1 or 2) and —Rb—S(O)tN(Ra)2 (where t is 1 or 2), where each Ra is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, amino or trifluoromethyl), or heteroarylalkyl (optionally substituted with halogen, hydroxy, methoxy, or trifluoromethyl), each Rb is independently a direct bond or a straight or branched alkylene or alkenylene chain, and Rc is a straight or branched alkylene or alkenylene chain, and where each of the Ra, Rb, or Rc substituents is unsubstituted unless otherwise indicated.
“Carbocyclylalkyl” refers to a radical of the formula -Rc-carbocyclyl where Rc is an alkylene chain as defined above. The alkylene chain and the carbocyclyl radical is optionally substituted as defined above.
“Carbocyclylalkynyl” refers to a radical of the formula -Rc-carbocyclyl where Rc is an alkynylene chain as defined above. The alkynylene chain and the carbocyclyl radical is optionally substituted as defined above.
“Carbocyclylalkoxy” refers to a radical bonded through an oxygen atom of the formula —O—Re-carbocyclyl where Re is an alkylene chain as defined above. The alkylene chain and the carbocyclyl radical is optionally substituted as defined above.
“Halo” or “halogen” refers to bromo, chloro, fluoro or iodo substituents.
“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.
“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic, or tetracyclic ring system, which optionally includes spiro, fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocyclyl radical includes 2-oxa-7-azaspiro[3.5]nonanyl. Unless stated otherwise specifically in the specification, the term “heterocyclyl” is meant to include heterocyclyl radicals as defined above that are optionally substituted by one or more substituents selected from optionally substituted alkyl, optionally substituted alkenyl, optionally substituted alkynyl, halo, fluoroalkyl, oxo, thioxo, cyano, nitro, —Rb—ORa, —Rb—OC(O)—Ra, —Rb—OC(O)—ORa, —Rb—OC(O)—N(Ra)₂, —Rb—N(Ra)₂, —Rb—C(O)Ra, —Rb—C(O)ORa, —Rb—C(O)N(Ra)₂, —Rb—O—Rc—C(O)N(Ra)₂, —Rb—N(Ra)C(O)ORa, —Rb—N(Ra)C(O)Ra, —Rb—N(Ra)S(O)tRa (where t is 1 or 2), —Rb—S(O)tRa (where t is 1 or 2), —Rb—S(O)tORa (where t is 1 or 2) and —Rb—S(O)tN(Ra)2 (where t is 1 or 2), where each Ra is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each Rb is independently a direct bond or a straight or branched alkylene or alkenylene chain, and Rc is a straight or branched alkylene or alkenylene chain, and where each of the Ra, Rb, or Rc substituents is unsubstituted unless otherwise indicated.
“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, and imidazolidinyl.
“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.
“Heterocyclylalkyl” refers to a radical of the formula —Rc-heterocyclyl where Rc is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.
“Heterocyclylalkoxy” refers to a radical bonded through an oxygen atom of the formula —O—Rc-heterocyclyl where Rc is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3-to 18-membered aromatic ring radical that comprises two to seventeen carbon atoms and from one to six heteroatoms selected from nitrogen, oxygen, and sulfur. As used herein, the heteroaryl radical is a monocyclic, bicyclic, tricyclic, or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl,benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). In some embodiments, the heteroaryl includes 5,6-dihydro-4H-pyrrolo[1,2-b]pyrazol-3-yl and 6,7-dihydro-5H-pyrazolo[5,1-b][1,3]oxazin-3-yl. In some embodiments, the heteroaryl includes 4,5,6,7-tetrahydropyrazolo[1,5-a]pyrazine. Unless stated otherwise specifically in the specification, the term “heteroaryl” is meant to include heteroaryl radicals as defined above which are optionally substituted by one or more substituents selected from optionally substituted alkyl, optionally substituted cycloalkylalkyl, optionally substituted heterocyclylalkyl, optionally substituted alkenyl, optionally substituted alkynyl, halo, optionally substituted fluoroalkyl, optionally substituted haloalkenyl, optionally substituted haloalkynyl, oxo, thioxo, cyano, nitro, —Rb—ORa, —Rb—OC(O)—Ra, —Rb—OC(O)—ORa, —Rb—OC(O)—N(Ra)₂, —Rb—N(Ra)₂, —Rb—C(O)Ra, —Rb—C(O)ORa, —Rb—C(O)N(Ra)₂, —Rb—O—Re—C(O)N(Ra)₂, —Rb—N(Ra)C(O)ORa, —Rb—N(Ra)C(O)Ra, —Rb—N(Ra)S(O)tRa (where t is 1 or 2), —Rb—S(O)tRa (where t is 1 or 2), —Rb—S(O)tORa (where t is 1 or 2) and —Rb—S(O)tN(Ra)2 (where t is 1 or 2), where each Ra is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each Rb is independently a direct bond or a straight or branched alkylene or alkenylene chain, and Rc is a straight or branched alkylene or alkenylene chain, and where each of the Ra, Rb, or Rc substituents is unsubstituted unless otherwise indicated.
“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.
“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.
“Heteroarylalkyl” refers to a radical of the formula —Rc-heteroaryl, where Rc is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.
“Heteroarylalkoxy” refers to a radical bonded through an oxygen atom of the formula —O—Rc-heteroaryl, where Rc is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

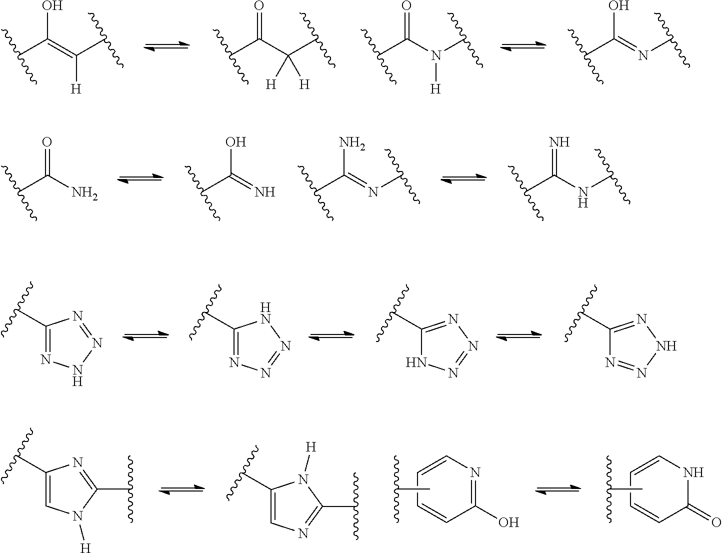

The compounds disclosed herein, in some embodiments, are used in different enriched isotopic forms, e.g., enriched in the content of 2H, 3H, 11C, 13C and/or 14C. In one particular embodiment, the compound is deuterated in at least one position. Such deuterated forms can be made by the procedure described in U.S. Pat. Nos. 5,846,514, 6,334,997. As described in U.S. Pat. Nos. 5,846,514, 6,334,997, deuteration can improve the metabolic stability and or efficacy, thus increasing the duration of action of drugs. Such compounds are referred to herein as deuteroisotope.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by 13C- or 14C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (2H), tritium (3H), iodine-125 (125I) or carbon-14 (14C). Isotopic substitution with 2H, 11C, 13C, 14C, 15C, 12N, 13N, 15N, 16N, 160, 170, 14F, 15F, 16F, 17F, 18F, 33S, 34S, 35S, 36S, 35C1, 37C1, 79Br, 81Br, 125I are all contemplated. In some embodiments, isotopic substitution with 18F is contemplated. All isotopic variations of the compounds of the present invention, whether radioactive or not, are encompassed within the scope of the present invention.

In certain embodiments, the compounds disclosed herein have some or all of the 1H atoms replaced with 2H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds.

Large numbers of deuterium-containing reagents and building blocks are available commercially from chemical vendors, such as Aldrich Chemical Co.

Deuterium-transfer reagents suitable for use in nucleophilic substitution reactions, such as iodomethane-d3 (CD3I), are readily available and may be employed to transfer a deuterium-substituted carbon atom under nucleophilic substitution reaction conditions to the reaction substrate. The use of CD3I is illustrated, by way of example only, in the reaction schemes below.

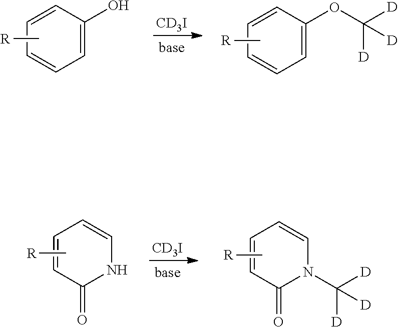

Deuterium-transfer reagents, such as lithium aluminum deuteride (LiAlD4), are employed to transfer deuterium under reducing conditions to the reaction substrate. The use of LiAlD4 is illustrated, by way of example only, in the reaction schemes below.

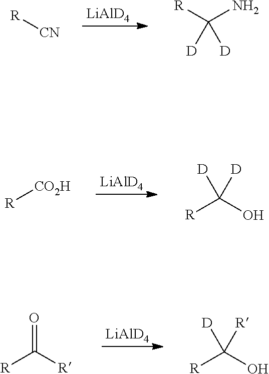

Deuterium gas and palladium catalyst are employed to reduce unsaturated carbon-carbon linkages and to perform a reductive substitution of aryl carbon-halogen bonds as illustrated, by way of example only, in the reaction schemes below.

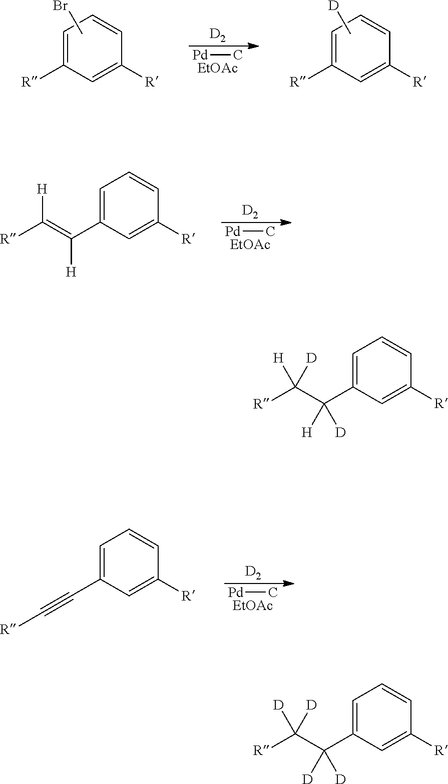

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable 1H hydrogen atoms. In one embodiment, the level of deuterium incorporation is determined by synthetic methods in which a deuterated synthetic building block is used as a starting material.

“A pharmaceutically acceptable salt” includes both acid and base addition salts. A pharmaceutically acceptable salt of any one of the PARG inhibitory compounds described herein is intended to encompass any and all pharmaceutically suitable salt forms. Preferred a pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid,p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like.

Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein exist in either unsolvated or solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### Poly(ADP-ribose) Glycohydrolase

A hallmark of cancer cells is increased levels of damaged DNA and associated replication stress, which can be defined as the slowing or stalling of replication forks during the DNA replication process. The cellular response to damaged DNA and replication stress is the activation of cell-cycle checkpoints and DNA damage response (DDR) mechanism s to arrest the cell cycle and promote repair of the damaged DNA via mechanisms that include single stranded break repair (SSBR) and base excision repair (BER) pathways. DDR and replication stress response represent cancer-specific vulnerabilities, which can be targeted to induce cancer cell senescence or death.

DNA damage and replication stress responses are regulated by poly ADP ribosylation (PARylation), a transient post-translational modification characterized by the polymerization of ADP-ribose molecules onto nuclear proteins to form poly(ADP-ribose) (PAR) chains. PARylation is catalyzed by the PARP (poly ADP-ribose polymerase) family of proteins via the hydrolysis of NAD+ into nicotinamide and ADP-ribose and the polymerization of the ADP-ribose onto acceptor proteins. PAR chain removal is catalyzed by members of the glycohydrolase family of enzymes of which PARG is the primary PAR glycohydrolase and accounts for approximately 90% of the dePARylation activity in vivo (Koh, 2004). PARG has a critical role in the DNA damage repair cycle and is required to complete the DNA break repair cycle initiated by PARP enzymes.

PARG exists as a single gene with isoforms that reside in the nucleus, mitochondria, and cytosol. In humans, the PARG gene is located on a single chromosome, at the locus 10q11.23-21, but can be subjected to alternative splicing, resulting in different PARG isoforms. Different PARG isoforms can be localized in different subcellular locations and have different degrees of catalytic activity.

In addition to its role in DNA damage response, PARG impacts PAR signaling in splicing, transcriptional, and epigenetic pathways. PARG can prevent the accumulation of cytoplasmic PAR, and also parthanatos, a PAR-mediated type of cell death. PARG functions to maintain stable levels of PAR to protect the cell against parthanatos, which is triggered by the release of the apoptosis-inducing factor (AIF) from the mitochondria to the nucleus. PARG can also have a functional role in telomere maintenance and replication by negatively regulating access to telomeric DNA and reversing ADP-ribosylation of telomeric-specific protein TRF1.

### Inhibition of PARG Function

Suppression of PAR chain hydrolysis via PARG depletion or inhibition leads to defective single-strand and double-strand break repair, reduced kinetics of break repair, and hypersensitivity to DNA damaging agents (Ame, 2009; Fisher, 2007; Gravells, 2017; Gravells, 2018, Kassab, 2020). PARG depletion or inhibition has been shown to inhibit proliferation and arrest cells in the S or G2 phase of the cell cycle and/or induce apoptosis alone or in combination with DNA damaging agents or replication stress inducers. Although PARG and PARP1 work in concert to regulate DNA SSB repair, PARG inhibitors show distinct pharmacology as compared to PARP inhibitors in some cancer cells, suggesting that PARG inhibitors could be used to treat a different subset of tumors as compared to PARP inhibitors. PARG depletion reduces survival of BRCA2-deficient cancer cells. PARG inhibitor sensitivity can also occur in BRCA wild-type, HR-proficient cells, and in PARP inhibitor resistant cells (Coulson-Gilmer, 2021; Pillay, 2019; Houl, 2019; James, 2016). Additionally, depletion of TIMELESS or DNA polymerase R (polo) showed synthetic lethality with PARG inhibition, but not PARP inhibition, in ovarian cancer cell lines (Pillay, 2019; Pillay, 2021; Ali, 2021).

PARG depletion can sensitize lung, cervical and pancreatic cancer cells to y-irradiation or experimental DNA damaging agents (e.g., hydrogen peroxide, methylmethanesulfonate) (Ame, Fouquerel et al. 2009) (Nakadate, Kodera et al. 2013) (Shirai, Poetsch et al. 2013). Deficiency in PARG does not sensitize to all agents (e.g., gemcitabine, camptothecin), indicating a specificity for PARG function with certain pathways of DNA damage repair and chemo- and radiotherapies (Fujihara, Ogino et al. 2009) (Shirai, Fujimori et al. 2013) (Zhou, Feng et al. 2010) (Zhou, Feng et al. 2011).

The unique efficacy of PARG inhibition in certain cancer types may occur because PARG inhibition appears to exploit specific deficiencies in replication fork machinery in cancer cells under conditions of replication stress (Pillay, 2019, Harrision, 2020). PARG inhibition further slows replication fork progression, increases fork stalling, and increases the number of reversed forks (Houl, 2019; Pillay, 2019; Slade, 2020). In support of the replication catastrophe hypothesis, treatment of sensitive cells with the PARG inhibitor PDD00017273 results in an S-phase dependent accumulation of nuclear RPA protein and induction of pan-nuclear QH2AX expression, hallmarks of replication catastrophe (Pillay, 2019; Toledo, 2013).

Inhibition of PARG activity can also be cytotoxic to sensitive cancer cells via additional mechanisms that include necrosis via cellular NAD+ depletion and parthanatos, a PARP-dependent, PAR-mediated, and caspase-independent form of cell death triggered by the nuclear translocation of apoptosis-inducing factor (AIF) and induction of DNA fragmentation (Feng, 2012; Nagashima, 2020; Zhou, 2011). Depletion of PARG, in contrast to PARP depletion, can lead to a drop in NAD levels, resulting in lung cancer cell death as a result of energy failure (Erdelyi, Bai et al. 2009).

In summary, PARG inhibitors can have uses as a cancer treatment both as single agents and in combination with therapeutic agents and radiotherapy. PARG inhibition leads to antitumor efficacy in cancer types that rely on the mechanisms of SSBR, BER, and protection of stalled replication forks under conditions of replication stress, and these mechanisms represent cancer-specific vulnerabilities. PARG inhibition can be used to target cancer cells having background genetic deficiencies that confer sensitivity to a replication catastrophe mechanism of cell death. PARG inhibition can be effective as a therapy against tumors that are resistant to other treatments such as PARP inhibitors and platinum therapies.

### Prior Art PARG Inhibitors

PARG inhibitors have not been studied to the extent of PARP inhibitors. Clinical resistance to PARP inhibitors has already been described and therefore there is a need to discover alternative inhibitors targeting the DNA damage repair pathways. Previously described PARG inhibitors include bicyclic aryl and heteroaryl compounds as described in WO 2016/092326, WO 2016/097749, WO 2021/055744, WO 2018/237296, WO 2020/023802, WO 2020/205646, WO 2022/138812, WO 2023/057389, WO 2023/057394, WO 2023/154913, WO 2023/165571, WO 2023/175184, WO 2023/175185, WO 2023/183850, WO2023/205914, and WO2023208092. There remains a need to find alternative PARG inhibitors, which can target DNA damage response pathways and can be cancer therapeutics. Novel PARG inhibitors have potential, as single agents or in combination with other therapeutics, for the treatment of cancers that do not respond, or have become resistant to, other therapeutics, such as PARP inhibitors and platinum-based therapeutics.

### PARG Inhibitory Compounds

In one aspect, provided herein is a PARG inhibitory compound.

One embodiment provides a compound, or a pharmaceutically acceptable salt, stereoisomer, or deuteroisotope thereof, having the structure of Formula (I):

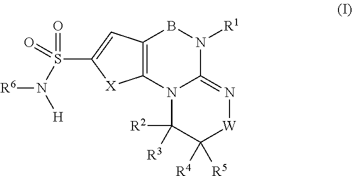

wherein,
B is a bond, C═O, or CN—OR⁹;
W is a bond, —O—, or C(R¹⁰)(R¹¹), or NR⁷;
X is —S—, or —*Y═C(A)—; wherein the * indicates point of attachment to C—SO₂ group;
Y is N, C—H, C—F, C—O(optionally substituted C1-C6 alkyl), C—NH₂, C—NH(optionally substituted C1-C6 alkyl), C—N(optionally substituted C1-C6 alkyl)₂, C—SH, C—S(optionally substituted C1-C6 alkyl), C—NH(optionally substituted (heteroaryl)alkylene), —C—NH(optionally substituted (heteroaryl)alkynylene);
A is selected from the group consisting of hydrogen, halo, —OH, —CN, optionally substituted C1-C6 alkoxy, —N(R⁷)₂, optionally substituted C1-C6 alkyl, optionally substituted C2-C6 alkenyl, optionally substituted C2-C6 alkynyl, optionally substituted C3-C7 carbocyclyl, optionally substituted (carbocyclyl)alkylene, optionally substituted (carbocyclyl)alkynylene, optionally substituted heterocyclyl, optionally substituted (heterocyclyl)alkylene, optionally substituted heteroaryl, and optionally substituted aryl;
R¹ is selected from the group consisting of optionally substituted alkyl, optionally substituted alkenyl, optionally substituted alkynyl, optionally substituted (cycloalkyl)alkylene, optionally substituted (aryl)alkylene, optionally substituted (heterocyclyl)alkylene, optionally substituted (heteroaryl)alkylene, optionally substituted (heteroaryl)alkynylene, optionally substituted aryl, optionally substituted heteroaryl, optionally substituted C3-C7 carbocyclyl, and optionally substituted heterocyclyl;
R² and R³ are independently selected from the group consisting of hydrogen, halo, —OH, —CN, optionally substituted alkyl, optionally substituted alkenyl, optionally substituted alkynyl, optionally substituted cycloalkyl, optionally substituted aryl, optionally substituted heteroaryl, optionally substituted heterocyclyl, optionally substituted (cycloalkyl)alkylene, optionally substituted (heterocyclyl)alkylene, optionally substituted (aryl)alkylene, optionally substituted (heteroaryl)alkylene, optionally substituted (cycloalkyl)alkenylene, optionally substituted (heterocyclyl)alkenylene, optionally substituted (aryl)alkenylene, optionally substituted (heteroaryl)alkenylene, optionally substituted (cycloalkyl)alkynylene, optionally substituted (heterocyclyl)alkynylene, optionally substituted (heteroaryl)alkynylene, optionally substituted (aryl)alkynylene, optionally substituted (cycloalkyl)-O-alkylene, optionally substituted (heterocyclyl)-O-alkylene, optionally substituted (aryl)-O-alkylene, optionally substituted (heteroaryl)-O-alkylene, optionally substituted (cycloalkyl)-NR⁷-alkylene, optionally substituted (heterocyclyl)-NR⁷-alkylene, optionally substituted (aryl)-NR⁷-alkylene, optionally substituted (heteroaryl)-NR⁷-alkylene, optionally substituted (alkyl)-O-alkylene, optionally substituted (alkyl)-NR⁷-alkylene, optionally substituted (alkyl)-O-alkynylene, and optionally substituted (alkyl)-NR⁷-alkynylene; or
optionally, R² and R³ combine to form an optionally substituted carbocyclic or heterocyclic ring;
R⁴, R¹, R¹⁰, and R¹¹ are independently selected from the group consisting of hydrogen, halo, —OH, —CN, optionally substituted alkyl, optionally substituted alkenyl, optionally substituted alkynyl, optionally substituted cycloalkyl, optionally substituted (cycloalkyl)alkylene, optionally substituted (cycloalkyl)alkynylene, optionally substituted heterocyclyl, optionally substituted (heterocyclyl)alkylene, optionally substituted heteroaryl, and optionally substituted aryl;
or optionally, R⁴ and R⁵ combine to form an optionally substituted carbocyclic or heterocyclic ring;
or optionally, R³ and R⁴ combine to form an optionally substituted carbocyclic or heterocyclic ring;
R⁶ is optionally substituted alkyl, optionally substituted cycloalkyl, optionally substituted heterocyclyl, or an optionally substituted 1,1′-bi(cyclopropan)-1-yl;
each R⁷ is independently hydrogen or an optionally substituted C1-C6 alkyl; and
each R⁹ is independently hydrogen or an optionally substituted C1-C6 alkyl.

One embodiment provides a compound, or a pharmaceutically acceptable salt, stereoisomer, or deuteroisotope thereof, having the structure of Formula (Ia):

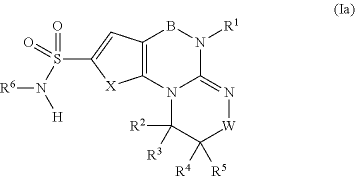

wherein,
B is a bond, C═O, or CN—OR⁹;
W is a bond, —O—, or C(R¹⁰)(R¹¹), or NR⁷;
X is —S—, or —Y═C(A)—;
Y is N, C—H, or C—F;
A is selected from the group consisting of hydrogen, halo, —OH, —CN, optionally substituted C1-C6 alkoxy, —N(R⁷)₂, optionally substituted C1-C6 alkyl, optionally substituted C2-C6 alkenyl, optionally substituted C2-C6 alkynyl, optionally substituted C3-C7 carbocyclyl, optionally substituted (carbocyclyl)alkylene, optionally substituted (carbocyclyl)alkynylene, optionally substituted heterocyclyl, optionally substituted (heterocyclyl)alkylene, optionally substituted heteroaryl, and optionally substituted aryl;
R¹ is selected from the group consisting of optionally substituted alkyl, optionally substituted alkenyl, optionally substituted alkynyl, optionally substituted (cycloalkyl)alkylene, optionally substituted (aryl)alkylene, optionally substituted (heterocyclyl)alkylene, optionally substituted (heteroaryl)alkylene, optionally substituted aryl, optionally substituted heteroaryl, optionally substituted C3-C7 carbocyclyl, and optionally substituted heterocyclyl;
R² and R³ are independently selected from the group consisting of hydrogen, halo, —OH, —CN, optionally substituted alkyl, optionally substituted alkenyl, optionally substituted alkynyl, optionally substituted cycloalkyl, optionally substituted aryl, optionally substituted heteroaryl, optionally substituted heterocyclyl, optionally substituted (cycloalkyl)alkylene, optionally substituted (heterocyclyl)alkylene, optionally substituted (aryl)alkylene, optionally substituted (heteroaryl)alkylene, optionally substituted (cycloalkyl)alkenylene, optionally substituted (heterocyclyl)alkenylene, optionally substituted (aryl)alkenylene, optionally substituted (heteroaryl)alkenylene, optionally substituted (cycloalkyl)alkynylene, optionally substituted (heterocyclyl)alkynylene, optionally substituted (heteroaryl)alkynylene, optionally substituted (aryl)alkynylene, optionally substituted (cycloalkyl)-O-alkylene, optionally substituted (heterocyclyl)-O-alkylene, optionally substituted (aryl)-O-alkylene, optionally substituted (heteroaryl)-O-alkylene, optionally substituted (cycloalkyl)-NR⁷-alkylene, optionally substituted (heterocyclyl)-NR⁷-alkylene, optionally substituted (aryl)-NR⁷-alkylene, optionally substituted (heteroaryl)-NR⁷-alkylene, optionally substituted (alkyl)-O-alkylene, optionally substituted (alkyl)-NR⁷-alkylene, optionally substituted (alkyl)-O-alkynylene, and optionally substituted (alkyl)-NR⁷-alkynylene; or
optionally, R² and R³ combine to form an optionally substituted carbocyclic or heterocyclic ring;
R⁴, R¹, R¹⁰, and R¹¹ are independently selected from the group consisting of hydrogen, halo, —OH, —CN, optionally substituted alkyl, optionally substituted alkenyl, optionally substituted alkynyl, optionally substituted cycloalkyl, optionally substituted (cycloalkyl)alkylene, optionally substituted (cycloalkyl)alkynylene, optionally substituted heterocyclyl, optionally substituted (heterocyclyl)alkylene, optionally substituted heteroaryl, and optionally substituted aryl;
or optionally, R⁴ and R⁵ combine to form an optionally substituted carbocyclic or heterocyclic ring;
or optionally, R³ and R⁴ combine to form an optionally substituted carbocyclic or heterocyclic ring;
R⁶ is optionally substituted alkyl, optionally substituted cycloalkyl, optionally substituted heterocyclyl, or an optionally substituted 1,1′-bi(cyclopropan)-1-yl;
each R⁷ is independently hydrogen or an optionally substituted C1-C6 alkyl; and
each R⁹ is independently hydrogen or an optionally substituted C1-C6 alkyl.

One embodiment provides the compound of Formula (I), or a pharmaceutically acceptable salt, stereoisomer, or deuteroisotope thereof, wherein X is -S-.

One embodiment provides the compound of Formula (I), or a pharmaceutically acceptable salt, stereoisomer, or deuteroisotope thereof, wherein Y is N. One embodiment provides the compound of Formula (I), or a pharmaceutically acceptable salt, stereoisomer, or deuteroisotope thereof, wherein Y is C—H. One embodiment provides the compound of Formula (I), or a pharmaceutically acceptable salt, stereoisomer, or deuteroisotope thereof, wherein Y is C—F. One embodiment provides the compound of Formula (I), or a pharmaceutically acceptable salt, stereoisomer, or deuteroisotope thereof, wherein A is hydrogen. One embodiment provides the compound of Formula (I), or a pharmaceutically acceptable salt, stereoisomer, or deuteroisotope thereof, wherein A is halo or -CN. One embodiment provides the compound of Formula (I), or a pharmaceutically acceptable salt, stereoisomer, or deuteroisotope thereof, wherein A is optionally substituted C1-C6 alkoxy or —N(R7)2. One embodiment provides the compound of Formula (I), or a pharmaceutically acceptable salt, stereoisomer, or deuteroisotope thereof, wherein A is selected from the group consisting optionally substituted C1-C6 alkyl, optionally substituted C3-C7 carbocyclyl, and optionally substituted (carbocyclyl)alkylene. One embodiment provides the compound of Formula (I), or a pharmaceutically acceptable salt, stereoisomer, or deuteroisotope thereof, wherein A is optionally substituted heterocyclyl or optionally substituted (heterocyclyl)alkylene. One embodiment provides the compound of Formula (I), or a pharmaceutically acceptable salt, stereoisomer, or deuteroisotope thereof, wherein A is selected from optionally substituted heteroaryl or optionally substituted aryl.

One embodiment provides the compound of Formula (I), or a pharmaceutically acceptable salt, stereoisomer, or deuteroisotope thereof, having the structure of Formula (II):

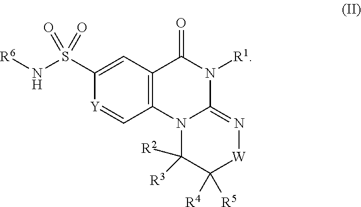

One embodiment provides the compound of Formula (II), or a pharmaceutically acceptable salt, stereoisomer, or deuteroisotope thereof, wherein W is a bond. One embodiment provides the compound of Formula (II), or a pharmaceutically acceptable salt, stereoisomer, or deuteroisotope thereof, wherein W is O. One embodiment provides the compound of Formula (II), or a pharmaceutically acceptable salt, stereoisomer, or deuteroisotope thereof, wherein W is CR10R11.

One embodiment provides the compound of Formula (I), or a pharmaceutically acceptable salt, stereoisomer, or deuteroisotope thereof, having the structure of Formula (III):

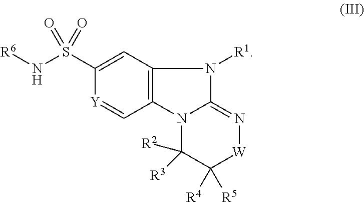

One embodiment provides the compound of Formula (III), or a pharmaceutically acceptable salt, stereoisomer, or deuteroisotope thereof, wherein W is a bond. One embodiment provides the compound of Formula (III), or a pharmaceutically acceptable salt, stereo isomer, or deuteroisotope thereof, wherein W is O. One embodiment provides the compound of Formula (III), or a pharmaceutically acceptable salt, stereoisomer, or deuteroisotope thereof, wherein W is CR10R11.

One embodiment provides the compound of Formula (III), or a pharmaceutically acceptable salt, stereoisomer, or deuteroisotope thereof, wherein Y is N. One embodiment provides the compound of Formula (III), or a pharmaceutically acceptable salt, stereoisomer, or deuteroisotope thereof, wherein Y is C—H. One embodiment provides the compound of Formula (III), or a pharmaceutically acceptable salt, stereoisomer, or deuteroisotope thereof, wherein Y is C—F. One embodiment provides the compound of Formula (III), or a pharmaceutically acceptable salt, stereoisomer, or deuteroisotope thereof, wherein A is hydrogen. One embodiment provides the compound of Formula (III), or a pharmaceutically acceptable salt, stereoisomer, or deuteroisotope thereof, wherein A is halo or —CN. One embodiment provides the compound of Formula (III), or a pharmaceutically acceptable salt, stereoisomer, or deuteroisotope thereof, wherein A is optionally substituted C1-C6 alkoxy or —N(R7)2. One embodiment provides the compound of Formula (III), or a pharmaceutically acceptable salt, stereoisomer, or deuteroisotope thereof, wherein A is selected from the group consisting optionally substituted C1-C6 alkyl, optionally substituted C3-C7 carbocyclyl, and optionally substituted (carbocyclyl)alkylene. One embodiment provides the compound of Formula (III), or a pharmaceutically acceptable salt, stereoisomer, or deuteroisotope thereof, wherein A is optionally substituted heterocyclyl or optionally substituted (heterocyclyl)alkylene. One embodiment provides the compound of Formula (III), or a pharmaceutically acceptable salt, stereoisomer, or deuteroisotope thereof, wherein A is selected from optionally substituted heteroaryl or optionally substituted aryl.

One embodiment provides the compound of Formula (I), or a pharmaceutically acceptable salt, stereoisomer, or deuteroisotope thereof, wherein R1 is optionally substituted alkyl. One embodiment provides the compound of Formula (I), or a pharmaceutically acceptable salt, stereoisomer, or deuteroisotope thereof, wherein R1 is optionally substituted C1-C4 alkyl.

One embodiment provides the compound of Formula (I), or a pharmaceutically acceptable salt, stereoisomer, or deuteroisotope thereof, wherein R1 is optionally substituted C1-C4 alkyl and is substituted with at least one substituent selected from —CN, —OR9, halo, oxo, —N(R9)2, or -CON(R9)2; wherein each R9 is independently hydrogen, or optionally substituted C1-C4 alkyl.

One embodiment provides the compound of Formula (I), or a pharmaceutically acceptable salt, stereoisomer, or deuteroisotope thereof, wherein R1 is optionally substituted (heteroaryl)alkylene. One embodiment provides the compound of Formula (I), or a pharmaceutically acceptable salt, stereoisomer, or deuteroisotope thereof, wherein the optionally substituted heteroaryl is selected from a 5- or a 6-membered heteroaryl. One embodiment provides the compound of Formula (I), or a pharmaceutically acceptable salt, stereoisomer, or deuteroisotope thereof, wherein the heteroaryl is selected from a 5-membered nitrogen-containing heteroaryl. One embodiment provides the compound of Formula (I), or a pharmaceutically acceptable salt, stereoisomer, or deuteroisotope thereof, wherein the optionally substituted 5-membered nitrogen-containing heteroaryl is selected from optionally substituted thiazole, optionally substituted oxazole, optionally substituted imidazole, optionally substituted pyrazole, optionally substituted isoxazole, optionally substituted pyrrole, optionally substituted oxadiazole, optionally substituted triazole, optionally substituted thiadiazole, or optionally substituted isothiazole or optionally substituted isoxazole. One embodiment provides the compound of Formula (I), or a pharmaceutically acceptable salt, stereoisomer, or deuteroisotope thereof, wherein the optionally substituted 5-membered nitrogen-containing heteroaryl is selected from optionally substituted pyrazole or optionally substituted isoxazole.

One embodiment provides the compound of Formula (I), or a pharmaceutically acceptable salt, stereoisomer, or deuteroisotope thereof, wherein the heteroaryl is a 6-membered nitrogen-containing heteroaryl.

One embodiment provides the compound of Formula (I), or a pharmaceutically acceptable salt, stereoisomer, or deuteroisotope thereof, wherein the 6-membered nitrogen-containing heteroaryl is an optionally substituted pyridine or optionally substituted pyrazine.

One embodiment provides the compound of Formula (I), or a pharmaceutically acceptable salt, stereoisomer, or deuteroisotope thereof, wherein R1 is optionally substituted heteroaralkyl and the alkylene is an optionally substituted C1-C4 alkylene. One embodiment provides the compound of Formula (I), or a pharmaceutically acceptable salt, stereoisomer, or deuteroisotope thereof, wherein the optionally substituted C1-C4 alkylene is a —CH2—. One embodiment provides the compound of Formula (I), or a pharmaceutically acceptable salt, stereoisomer, or deuteroisotope thereof, wherein the optionally substituted C1-C4 alkylene is a -CD2-. One embodiment provides the compound of Formula (I), or a pharmaceutically acceptable salt, stereoisomer, or deuteroisotope thereof, wherein R1 is optionally substituted (aryl)alkylene. One embodiment provides the compound of Formula (I), or a pharmaceutically acceptable salt, stereoisomer, or deuteroisotope thereof, wherein the optionally substituted (aryl)alkylene comprises an optionally substituted phenyl. One embodiment provides the compound of Formula (I), or a pharmaceutically acceptable salt, stereoisomer, or deuteroisotope thereof, wherein the optionally substituted (aryl)alkylene comprises an optionally substituted C1-C4 alkylene.

One embodiment provides the compound of Formula (I), or a pharmaceutically acceptable salt, stereoisomer, or deuteroisotope thereof, wherein the optionally substituted C1-C4 alkylene is a —CH2— or —CD2—. One embodiment provides the compound of Formula (I), or a pharmaceutically acceptable salt, stereoisomer, or deuteroisotope thereof, wherein R1 is an optionally substituted C4-C7 (carbocyclyl)alkylene. One embodiment provides the compound of Formula (I), or a pharmaceutically acceptable salt, stereoisomer, or deuteroisotope thereof, wherein the optionally substituted (carbocyclyl)alkylene is an optionally substituted (cyclopropyl)methylene. One embodiment provides the compound of Formula (I), or a pharmaceutically acceptable salt, stereoisomer, or deuteroisotope thereof, wherein R1 is an optionally substituted (heterocyclyl)alkylene. One embodiment provides the compound of Formula (I), or a pharmaceutically acceptable salt, stereoisomer, or deuteroisotope thereof, wherein the heteroaryl is selected from a 5-membered nitrogen-containing heteroaryl. One embodiment provides the compound of Formula (I), or a pharmaceutically acceptable salt, stereoisomer, or deuteroisotope thereof, wherein the optionally substituted 5-membered nitrogen-containing heteroaryl is selected from optionally substituted thiazole, optionally substituted oxazole, optionally substituted imidazole, optionally substituted pyrazole, optionally substituted isoxazole, optionally substituted pyrrole, optionally substituted oxadiazole, optionally substituted triazole, optionally substituted thiadiazole, or optionally substituted isothiazole or optionally substituted isoxazole. One embodiment provides the compound of Formula (I), or a pharmaceutically acceptable salt, stereoisomer, or deuteroisotope thereof, wherein the optionally substituted 5-membered nitrogen-containing heteroaryl is selected from optionally substituted thiadiazole or optionally substituted isoxazole.

One embodiment provides the compound of Formula (I), or a pharmaceutically acceptable salt, stereoisomer, or deuteroisotope thereof, wherein R4 is hydrogen or methyl.

One embodiment provides the compound of Formula (I), or a pharmaceutically acceptable salt, stereoisomer, or deuteroisotope thereof, wherein R5 is hydrogen or methyl.

One embodiment provides the compound of Formula (I), or a pharmaceutically acceptable salt, stereoisomer, or deuteroisotope thereof, wherein R3 is hydrogen or methyl.

One embodiment provides the compound of Formula (I), or a pharmaceutically acceptable salt, stereoisomer, or deuteroisotope thereof, wherein R2 is hydrogen.

One embodiment provides the compound of Formula (I), or a pharmaceutically acceptable salt, stereoisomer, or deuteroisotope thereof, wherein R2 is not hydrogen.

One embodiment provides the compound of Formula (I), or a pharmaceutically acceptable salt, stereoisomer, or deuteroisotope thereof, wherein R2 is an optionally substituted (heteroaryl)alkynylene. One embodiment provides the compound of Formula (I), or a pharmaceutically acceptable salt, stereoisomer, or deuteroisotope thereof, wherein the optionally substituted (heteroaryl)alkynyl is selected from:

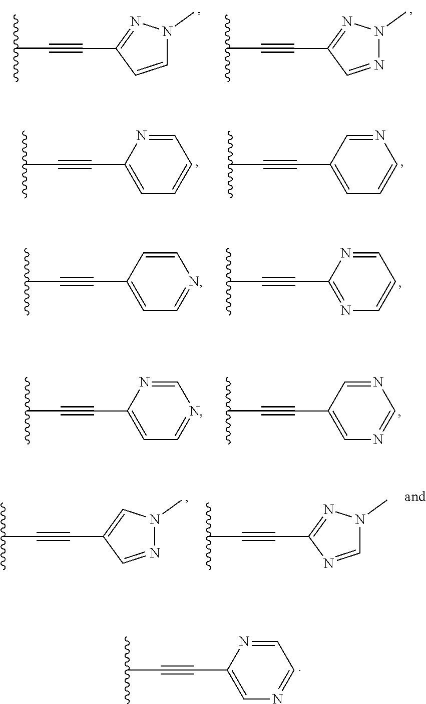

One embodiment provides the compound of Formula (I), or a pharmaceutically acceptable salt, stereoisomer, or deuteroisotope thereof, wherein R² is an optionally substituted (aryl)alkynylene.

One embodiment provides the compound of Formula (I), or a pharmaceutically acceptable salt, stereoisomer, or deuteroisotope thereof, wherein the optionally substituted (aryl)alkynylene is —C≡C—C6H₅.

One embodiment provides the compound of Formula (I), or a pharmaceutically acceptable salt, stereoisomer, or deuteroisotope thereof, wherein R² is an optionally substituted (aryl)alkenylene.

One embodiment provides the compound of Formula (I), or a pharmaceutically acceptable salt, stereoisomer, or deuteroisotope thereof, wherein the optionally substituted (aryl)alkenylene is —CH═CH—C6H₅.

One embodiment provides the compound of Formula (I), or a pharmaceutically acceptable salt, stereoisomer, or deuteroisotope thereof, wherein R² is an optionally substituted (aryl)-O-alkylene.

One embodiment provides the compound of Formula (I), or a pharmaceutically acceptable salt, stereoisomer, or deuteroisotope thereof, wherein the optionally substituted (aryl)-O-alkylene is

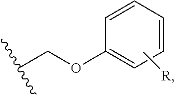

wherein R is selected from halo, —OH, —CN, optionally substituted C1-C6 alkoxy, or optionally substituted C1-C6 alkyl.

One embodiment provides the compound of Formula (I), or a pharmaceutically acceptable salt, stereoisomer, or deuteroisotope thereof, wherein R⁶ is optionally substituted C3-C5 cycloalkyl.

One embodiment provides the compound of Formula (I), or a pharmaceutically acceptable salt, stereoisomer, or deuteroisotope thereof, wherein the optionally substituted cycloalkyl is:

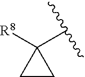

wherein R⁸ is selected from the group consisting of hydrogen, —CH₃, —CH₂F, —CHF₂, —CF₃, —CN, cyclopropyl, —CH₂CH₃, —CH(CH₃)₂, and —C(CH₃)3.

One embodiment provides the compound of Formula (I), or a pharmaceutically acceptable salt, stereoisomer, or deuteroisotope thereof, wherein B is CN—OR⁹.

One embodiment provides the compound of Formula (I), or a pharmaceutically acceptable salt, stereoisomer, or deuteroisotope thereof, wherein R⁹ is H. One embodiment provides the compound of Formula (I), or a pharmaceutically acceptable salt, stereoisomer, or deuteroisotope thereof, wherein R⁹ is optionally substituted C1-C3 alkyl.

One embodiment provides an PARG inhibitory compound, or a pharmaceutically acceptable salt or solvate thereof, having a structure presented in Table 1.

TABLE 1

| Synthetic | | |
|---|---|---|
| Chemistry | | |
| Example | Compound Structure | Compound Name |
| 1 | 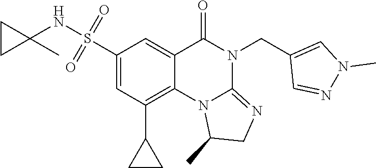 [SMILES: C[C@@H]1CN=C2N(Cc3cnn(C)c3)C(=O)c3cc(S(=O)(=O)NC4(C)CC4)cc(C4CC4)c3N21] | (R)-9-cyclopropyl-1-methyl-4- ((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 2 | 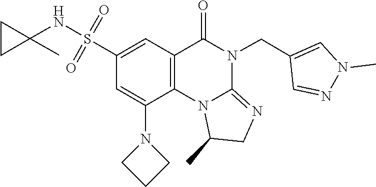 [SMILES: C[C@@H]1CN=C2N(Cc3cnn(C)c3)C(=O)c3cc(S(=O)(=O)NC4(C)CC4)cc(N4CCC4)c3N21] | (R)-9-(azetidin-1-yl)-1-methyl- 4-((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 3 | 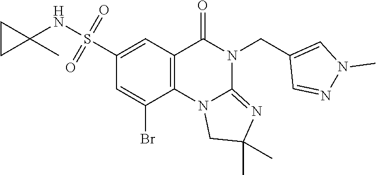 [SMILES: Cn1cc(CN2C(=O)c3cc(S(=O)(=O)NC4(C)CC4)cc(Br)c3N3CC(C)(C)N=C23)cn1] | 9-bromo-2,2-dimethyl-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 4 | 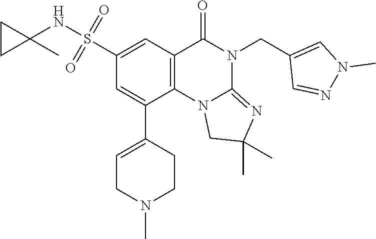 [SMILES: CN1CC=C(c2cc(S(=O)(=O)NC3(C)CC3)cc3c2N2CC(C)(C)N=C2N(Cc2cnn(C)c2)C3=O)CC1] | 2,2-dimethyl-9-(1-methyl- 1,2,3,6-tetrahydropyridin-4-yl)- 4-((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 5 | 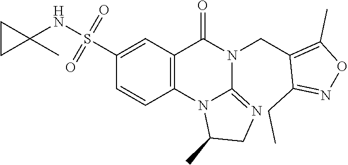 [SMILES: CCc1noc(C)c1CN1C(=O)c2cc(S(=O)(=O)NC3(C)CC3)ccc2N2C1=NC[C@H]2C] | (R)-4-((3-ethyl-5- methylisoxazol-4-yl)methyl)-1- methyl-N-(1- methylcyclopropyl)-5-oxo- 1,2,4,5-tetrahydroimidazo[1,2- a]quinazoline-7-sulfonamide |
| 6 | 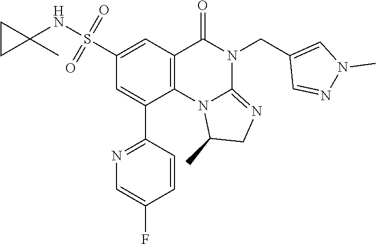 [SMILES: C[C@@H]1CN=C2N(Cc3cnn(C)c3)C(=O)c3cc(S(=O)(=O)NC4(C)CC4)cc(-c4ccc(F)cn4)c3N21] | (R)-9-(5-fluoropyridin-2-yl)-1- methyl-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 7 | 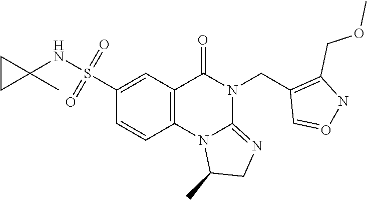 | (R)-4-((3- (methoxymethyl)isoxazol-4- yl)methyl)-1-methyl-N-(1- methylcyclopropyl)-5-oxo- 1,2,4,5-tetrahydroimidazo[1,2- a]quinazoline-7-sulfonamide |
| 8 | 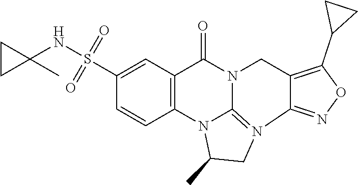 | (R)-4-((5-cyclopropyl-3- methylisoxazol-4-yl)methyl)-1- methyl-N-(1- methylcyclopropyl)-5-oxo- 1,2,4,5-tetrahydroimidazo[1,2- a]quinazoline-7-sulfonamide |
| 9 | 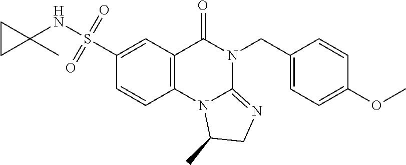 [SMILES: COc1ccc(CN2C(=O)c3cc(S(=O)(=O)NC4(C)CC4)ccc3N3C2=NC[C@H]3C)cc1] | (R)-4-(4-methoxybenzyl)-1- methyl-N-(1- methylcyclopropyl)-5-oxo- 1,2,4,5-tetrahydroimidazo[1,2- a]quinazoline-7-sulfonamide |
| 10 | 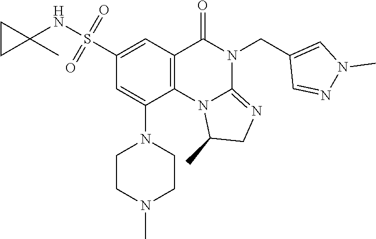 [SMILES: C[C@@H]1CN=C2N(Cc3cnn(C)c3)C(=O)c3cc(S(=O)(=O)NC4(C)CC4)cc(N4CCN(C)CC4)c3N21] | (R)-1-methyl-4-((1-methyl-1H- pyrazol-4-yl)methyl)-N-(1- methylcyclopropyl)-9-(4- methylpiperazin-1-yl)-5-oxo- 1,2,4,5-tetrahydroimidazo[1,2- a]quinazoline-7-sulfonamide |
| 11 | 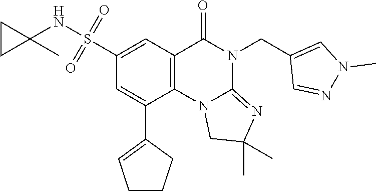 [SMILES: Cn1cc(CN2C(=O)c3cc(S(=O)(=O)NC4(C)CC4)cc(C4=CCCC4)c3N3CC(C)(C)N=C23)cn1] | 9-(cyclopent-1-en-1-yl)-2,2- dimethyl-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 12 | 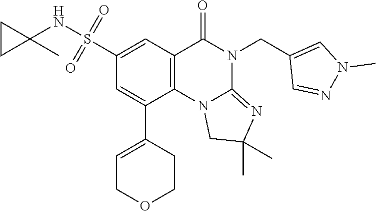 [SMILES: Cn1cc(CN2C(=O)c3cc(S(=O)(=O)NC4(C)CC4)cc(C4=CCOCC4)c3N3CC(C)(C)N=C23)cn1] | 9-(3,6-dihydro-2H-pyran-4-yl)- 2,2-dimethyl-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 13 | 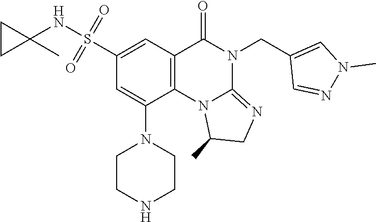 [SMILES: C[C@@H]1CN=C2N(Cc3cnn(C)c3)C(=O)c3cc(S(=O)(=O)NC4(C)CC4)cc(N4CCNCC4)c3N21] | (R)-1-methyl-4-((1-methyl-1H- pyrazol-4-yl)methyl)-N-(1- methylcyclopropyl)-5-oxo-9- (piperazin-1-yl)-1,2,4,5- tetrahydroimidazo[1,2- a]quinazoline-7-sulfonamide |
| 14 | 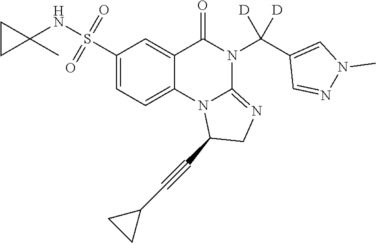 [SMILES: [2H]C([2H])(c1cnn(C)c1)N1C(=O)c2cc(S(=O)(=O)NC3(C)CC3)ccc2N2C1=NC[C@H]2C#CC1CC1] | (R)-1-(cyclopropylethynyl)-4- ((1-methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 15 | 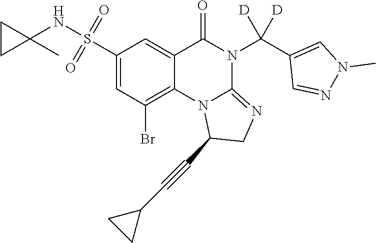 [SMILES: [2H]C([2H])(c1cnn(C)c1)N1C(=O)c2cc(S(=O)(=O)NC3(C)CC3)cc(Br)c2N2C1=NC[C@H]2C#CC1CC1] | (R)-9-bromo-1- (cyclopropylethynyl)-4-((1- methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 16 | 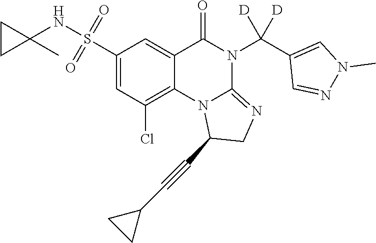 [SMILES: [2H]C([2H])(c1cnn(C)c1)N1C(=O)c2cc(S(=O)(=O)NC3(C)CC3)cc(Cl)c2N2C1=NC[C@H]2C#CC1CC1] | (R)-9-chloro-1- (cyclopropylethynyl)-4-((1- methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 17 | 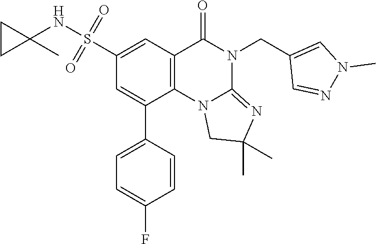 [SMILES: Cn1cc(CN2C(=O)c3cc(S(=O)(=O)NC4(C)CC4)cc(-c4ccc(F)cc4)c3N3CC(C)(C)N=C23)cn1] | 9-(4-fluorophenyl)-2,2- dimethyl-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 18 | 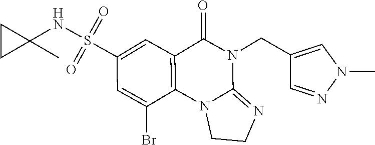 [SMILES: Cn1cc(CN2C(=O)c3cc(S(=O)(=O)NC4(C)CC4)cc(Br)c3N3CCN=C23)cn1] | 9-bromo-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 19 | 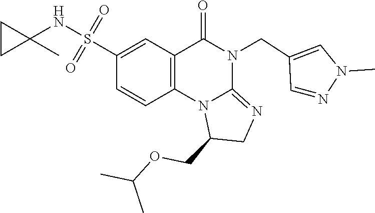 [SMILES: CC(C)OC[C@@H]1CN=C2N(Cc3cnn(C)c3)C(=O)c3cc(S(=O)(=O)NC4(C)CC4)ccc3N21] | (S)-1-(isopropoxymethyl)-4- ((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 20 | 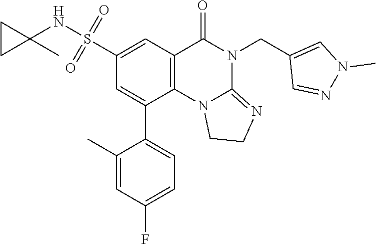 [SMILES: Cc1cc(F)ccc1-c1cc(S(=O)(=O)NC2(C)CC2)cc2c1N1CCN=C1N(Cc1cnn(C)c1)C2=O] | 9-(4-fluoro-2-methylphenyl)-4- ((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 21 | 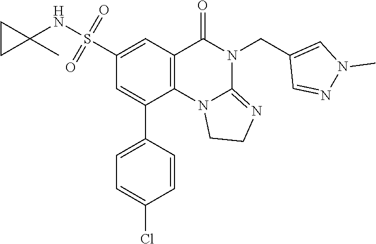 [SMILES: Cn1cc(CN2C(=O)c3cc(S(=O)(=O)NC4(C)CC4)cc(-c4ccc(Cl)cc4)c3N3CCN=C23)cn1] | 9-(4-chlorophenyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 22 | 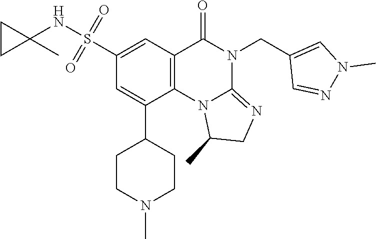 [SMILES: C[C@@H]1CN=C2N(Cc3cnn(C)c3)C(=O)c3cc(S(=O)(=O)NC4(C)CC4)cc(C4CCN(C)CC4)c3N21] | (R)-1-methyl-4-((1-methyl-1H- pyrazol-4-yl)methyl)-N-(1- methylcyclopropyl)-9-(1- methylpiperidin-4-yl)-5-oxo- 1,2,4,5-tetrahydroimidazo[1,2- a]quinazoline-7-sulfonamide |
| 23 | 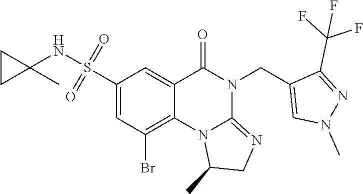 [SMILES: C[C@@H]1CN=C2N(Cc3cn(C)nc3C(F)(F)F)C(=O)c3cc(S(=O)(=O)NC4(C)CC4)cc(Br)c3N21] | (R)-9-bromo-1-methyl-4-((1- methyl-3-(trifluoromethyl)-1H- pyrazol-4-yl)methyl)-N-(1- methylcyclopropyl)-5-oxo- 1,2,4,5-tetrahydroimidazo[1,2- a]quinazoline-7-sulfonamide |
| 24 | 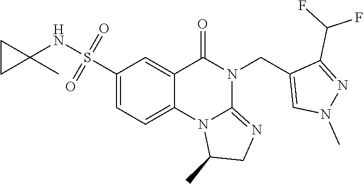 [SMILES: C[C@@H]1CN=C2N(Cc3cn(C)nc3C(F)F)C(=O)c3cc(S(=O)(=O)NC4(C)CC4)ccc3N21] | (R)-4-((3-(difluoromethyl)-1- methyl-1H-pyrazol-4- yl)methyl)-1-methyl-N-(1- methylcyclopropyl)-5-oxo- 1,2,4,5-tetrahydroimidazo[1,2- a]quinazoline-7-sulfonamide |
| 25 | 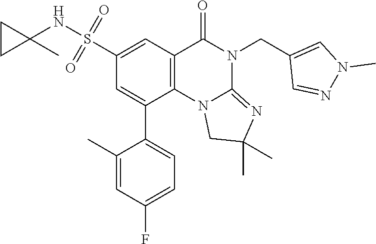 [SMILES: Cc1cc(F)ccc1-c1cc(S(=O)(=O)NC2(C)CC2)cc2c1N1CC(C)(C)N=C1N(Cc1cnn(C)c1)C2=O] | 9-(4-fluoro-2-methylphenyl)- 2,2-dimethyl-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 26 | 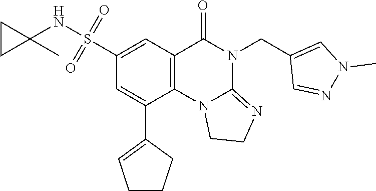 [SMILES: Cn1cc(CN2C(=O)c3cc(S(=O)(=O)NC4(C)CC4)cc(C4=CCCC4)c3N3CCN=C23)cn1] | 9-(cyclopent-1-en-1-yl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 27 | 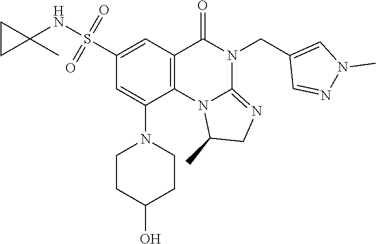 [SMILES: C[C@@H]1CN=C2N(Cc3cnn(C)c3)C(=O)c3cc(S(=O)(=O)NC4(C)CC4)cc(N4CCC(O)CC4)c3N21] | (R)-9-(4-hydroxypiperidin-1- yl)-1-methyl-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 28 | 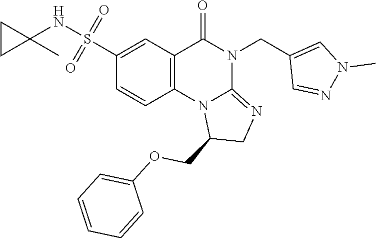 [SMILES: Cn1cc(CN2C(=O)c3cc(S(=O)(=O)NC4(C)CC4)ccc3N3C2=NC[C@H]3COc2ccccc2)cn1] | (S)-4-((1-methyl-1H-pyrazol-4- yl)methyl)-N-(1- methylcyclopropyl)-5-oxo-1- (phenoxymethyl)-1,2,4,5- tetrahydroimidazo[1,2- a]quinazoline-7-sulfonamide |
| 29 | 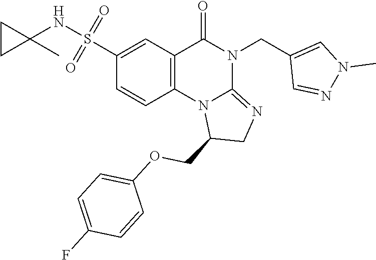 [SMILES: Cn1cc(CN2C(=O)c3cc(S(=O)(=O)NC4(C)CC4)ccc3N3C2=NC[C@H]3COc2ccc(F)cc2)cn1] | (S)-1-((4- fluorophenoxy)methyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 30 | 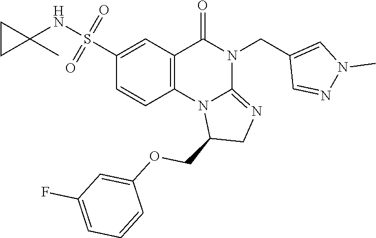 [SMILES: Cn1cc(CN2C(=O)c3cc(S(=O)(=O)NC4(C)CC4)ccc3N3C2=NC[C@H]3COc2cccc(F)c2)cn1] | (S)-1-((3- fluorophenoxy)methyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 31 | 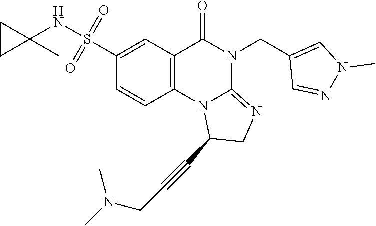 [SMILES: CN(C)CC#C[C@@H]1CN=C2N(Cc3cnn(C)c3)C(=O)c3cc(S(=O)(=O)NC4(C)CC4)ccc3N21] | (R)-1-(3-(dimethylamino)prop- 1-yn-1-yl)-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 32 | 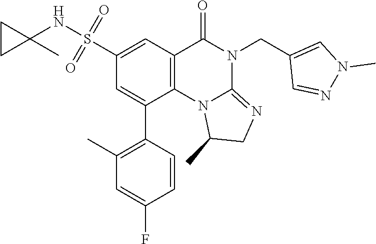 [SMILES: Cc1cc(F)ccc1-c1cc(S(=O)(=O)NC2(C)CC2)cc2c1N1C(=NC[C@H]1C)N(Cc1cnn(C)c1)C2=O] | (R)-9-(4-fluoro-2- methylphenyl)-1-methyl-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 33 | 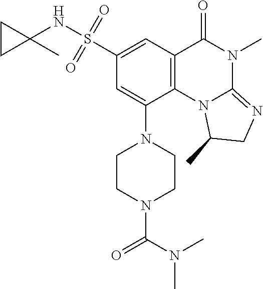 [SMILES: C[C@@H]1CN=C2N(C)C(=O)c3cc(S(=O)(=O)NC4(C)CC4)cc(N4CCN(C(=O)N(C)C)CC4)c3N21] | (R)-4-(1,4-dimethyl-7-(N-(1- methylcyclopropyl)sulfamoyl)- 5-oxo-1,2,4,5- tetrahydroimidazo[1,2- a]quinazolin-9-yl)-N,N- dimethylpiperazine-1- carboxamide |
| 34 | 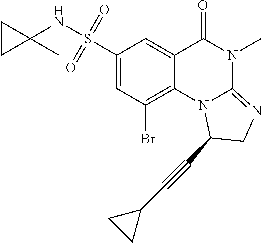 [SMILES: CN1C(=O)c2cc(S(=O)(=O)NC3(C)CC3)cc(Br)c2N2C1=NC[C@H]2C#CC1CC1] | (R)-9-bromo-1- (cyclopropylethynyl)-4-methyl- N-(1-methylcyclopropyl)-5- oxo-1,2,4,5- tetrahydroimidazo[1,2- a]quinazoline-7-sulfonamide |
| 35 | 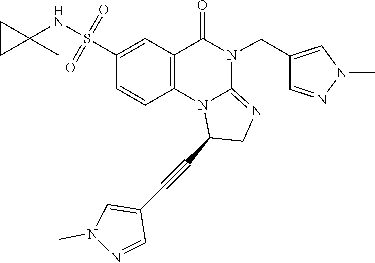 [SMILES: Cn1cc(C#C[C@@H]2CN=C3N(Cc4cnn(C)c4)C(=O)c4cc(S(=O)(=O)NC5(C)CC5)ccc4N32)cn1] | (R)-1-((1-methyl-1H-pyrazol-4- yl)ethynyl)-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 36 | 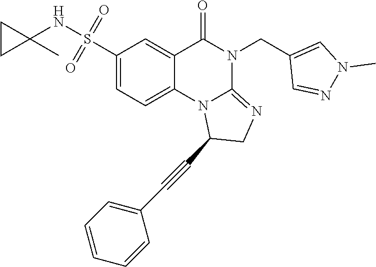 [SMILES: Cn1cc(CN2C(=O)c3cc(S(=O)(=O)NC4(C)CC4)ccc3N3C2=NC[C@H]3C#Cc2ccccc2)cn1] | (R)-4-((1-methyl-1H-pyrazol-4- yl)methyl)-N-(1- methylcyclopropyl)-5-oxo-1- (phenylethynyl)-1,2,4,5- tetrahydroimidazo[1,2- a]quinazoline-7-sulfonamide |
| 37 | 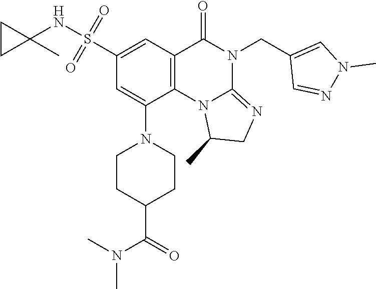 [SMILES: C[C@@H]1CN=C2N(Cc3cnn(C)c3)C(=O)c3cc(S(=O)(=O)NC4(C)CC4)cc(N4CCC(C(=O)N(C)C)CC4)c3N21] | (R)-N,N-dimethyl-1-(1-methyl- 4-((1-methyl-1H-pyrazol-4- yl)methyl)-7-(N-(1- methylcyclopropyl)sulfamoyl)- 5-oxo-1,2,4,5- tetrahydroimidazo[1,2- a]quinazolin-9-yl)piperidine-4- carboxamide |
| 38 | 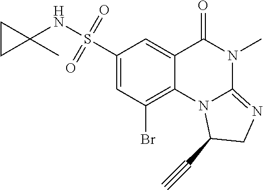 [SMILES: C#C[C@@H]1CN=C2N(C)C(=O)c3cc(S(=O)(=O)NC4(C)CC4)cc(Br)c3N21] | (R)-9-bromo-1-ethynyl-4- methyl-N-(1- methylcyclopropyl)-5-oxo- 1,2,4,5-tetrahydroimidazo[1,2- a]quinazoline-7-sulfonamide |
| 39 | 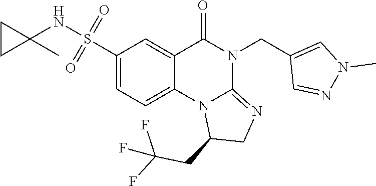 [SMILES: Cn1cc(CN2C(=O)c3cc(S(=O)(=O)NC4(C)CC4)ccc3N3C2=NC[C@H]3CC(F)(F)F)cn1] | (R)-4-((1-methyl-1H-pyrazol-4- yl)methyl)-N-(1- methylcyclopropyl)-5-oxo-1- (2,2,2-trifluoroethyl)-1,2,4,5- tetrahydroimidazo[1,2- a]quinazoline-7-sulfonamide |
| 40 | 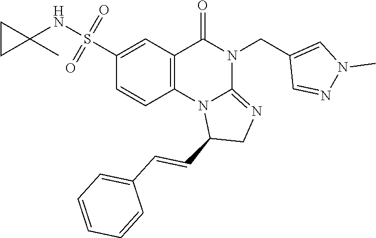 [SMILES: Cn1cc(CN2C(=O)c3cc(S(=O)(=O)NC4(C)CC4)ccc3N3C2=NC[C@H]3/C=C/c2ccccc2)cn1] | (R,E)-4-((1-methyl-1H-pyrazol- 4-yl)methyl)-N-(1- methylcyclopropyl)-5-oxo-1- styryl-1,2,4,5- tetrahydroimidazo[1,2- a]quinazoline-7-sulfonamide |
| 41 | 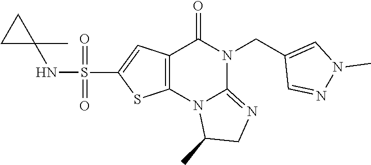 [SMILES: C[C@@H]1CN=C2N(Cc3cnn(C)c3)C(=O)c3cc(S(=O)(=O)NC4(C)CC4)sc3N21] | (R)-8-methyl-5-((1-methyl-1H- pyrazol-4-yl)methyl)-N-(1- methylcyclopropyl)-4-oxo- 4,5,7,8-tetrahydroimidazo[1,2- a]thieno[3,2-e]pyrimidine-2- sulfonamide |
| 42 | 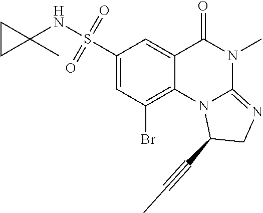 [SMILES: CC#C[C@@H]1CN=C2N(C)C(=O)c3cc(S(=O)(=O)NC4(C)CC4)cc(Br)c3N21] | (R)-9-bromo-4-methyl-N-(1- methylcyclopropyl)-5-oxo-1- (prop-1-yn-1-yl)-1,2,4,5- tetrahydroimidazo[1,2- a]quinazoline-7-sulfonamide |
| 43 | 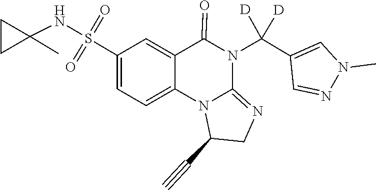 [SMILES: [2H]C([2H])(c1cnn(C)c1)N1C(=O)c2cc(S(=O)(=O)NC3(C)CC3)ccc2N2C1=NC[C@H]2C#C] | (R)-1-ethynyl-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 44 | 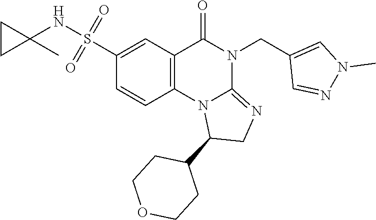 [SMILES: Cn1cc(CN2C(=O)c3cc(S(=O)(=O)NC4(C)CC4)ccc3N3C2=NC[C@H]3C2CCOCC2)cn1] | (R)-4-((1-methyl-1H-pyrazol-4- yl)methyl)-N-(1- methylcyclopropyl)-5-oxo-1- (tetrahydro-2H-pyran-4-yl)- 1,2,4,5-tetrahydroimidazo[1,2- a]quinazoline-7-sulfonamide |
| 45 | 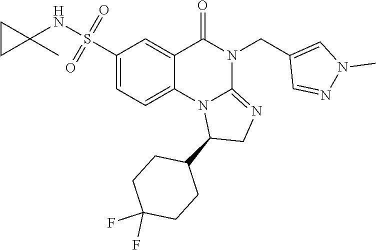 [SMILES: Cn1cc(CN2C(=O)c3cc(S(=O)(=O)NC4(C)CC4)ccc3N3C2=NC[C@H]3C2CCC(F)(F)CC2)cn1] | (R)-1-(4,4-difluorocyclohexyl)- 4-((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 46 | 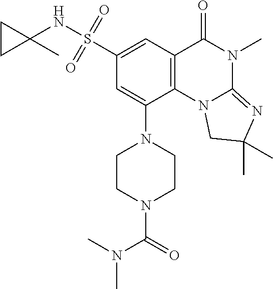 [SMILES: CN(C)C(=O)N1CCN(c2cc(S(=O)(=O)NC3(C)CC3)cc3c2N2CC(C)(C)N=C2N(C)C3=O)CC1] | N,N-dimethyl-4-(2,2,4- trimethyl-7-(N-(1- methylcyclopropyl)sulfamoyl)- 5-oxo-1,2,4,5- tetrahydroimidazo[1,2- a]quinazolin-9-yl)piperazine-1- carboxamide |
| 47 | 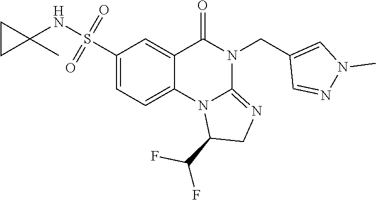 [SMILES: Cn1cc(CN2C(=O)c3cc(S(=O)(=O)NC4(C)CC4)ccc3N3C2=NC[C@H]3C(F)F)cn1] | (S)-1-(difluoromethyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 48 | 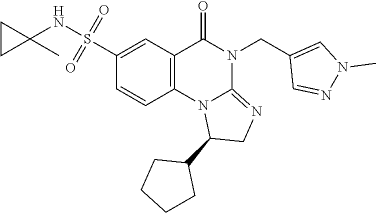 [SMILES: Cn1cc(CN2C(=O)c3cc(S(=O)(=O)NC4(C)CC4)ccc3N3C2=NC[C@H]3C2CCCC2)cn1] | (R)-1-cyclopentyl-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 49 | 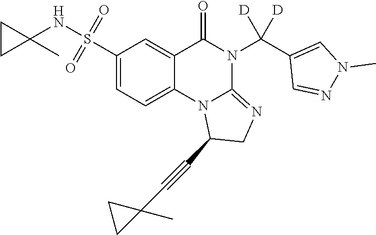 [SMILES: [2H]C([2H])(c1cnn(C)c1)N1C(=O)c2cc(S(=O)(=O)NC3(C)CC3)ccc2N2C1=NC[C@H]2C#CC1(C)CC1] | (R)-4-((1-methyl-1H-pyrazol-4- yl)methyl-d2)-N-(1- methylcyclopropyl)-1-((1- methylcyclopropyl)ethynyl)-5- oxo-1,2,4,5- tetrahydroimidazo[1,2- a]quinazoline-7-sulfonamide |
| 50 | 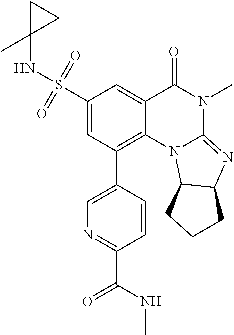 | N-methyl-5-((7aS,10aR)-6- methyl-3-(N-(1- methylcyclopropyl)sulfamoyl)- 5-oxo-6,7a,8,9,10,10a- hexahydro-5H- cyclopenta[4,5]imidazo[1,2- a]quinazolin-1-yl)picolinamide |
| 51 | 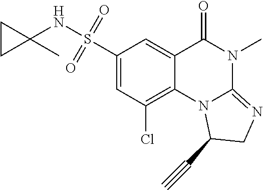 [SMILES: C#C[C@@H]1CN=C2N(C)C(=O)c3cc(S(=O)(=O)NC4(C)CC4)cc(Cl)c3N21] | (R)-9-chloro-1-ethynyl-4- methyl-N-(1- methylcyclopropyl)-5-oxo- 1,2,4,5-tetrahydroimidazo[1,2- a]quinazoline-7-sulfonamide |
| 52 | 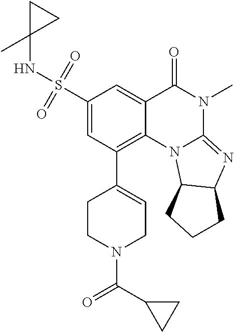 | (7aS,10aR)-1-(1- (cyclopropanecarbonyl)- 1,2,3,6-tetrahydropyridin-4-yl)- 6-methyl-N-(1- methylcyclopropyl)-5-oxo- 6,7a,8,9,10,10a-hexahydro-5H- cyclopenta[4,5]imidazo[1,2- a]quinazoline-3-sulfonamide |
| 53 | 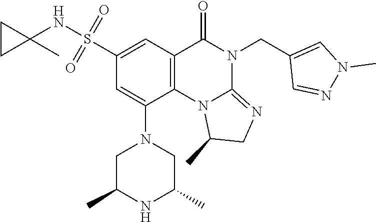 [SMILES: C[C@@H]1CN=C2N(Cc3cnn(C)c3)C(=O)c3cc(S(=O)(=O)NC4(C)CC4)cc(N4C[C@H](C)N[C@@H](C)C4)c3N21] | (R)-9-((3S,5S)-3,5- dimethylpiperazin-1-yl)-1- methyl-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 54 | 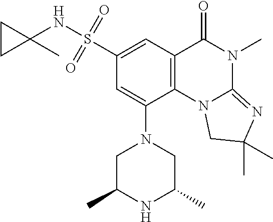 [SMILES: C[C@H]1CN(c2cc(S(=O)(=O)NC3(C)CC3)cc3c2N2CC(C)(C)N=C2N(C)C3=O)C[C@H](C)N1] | 9-((3S,5S)-3,5- dimethylpiperazin-1-yl)-2,2,4- trimethyl-N-(1- methylcyclopropyl)-5-oxo- 1,2,4,5-tetrahydroimidazo[1,2- a]quinazoline-7-sulfonamide |
| 55 | 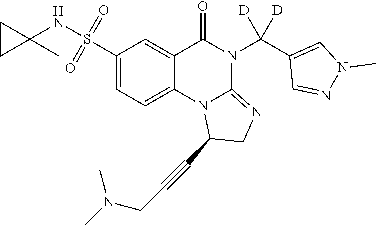 [SMILES: [2H]C([2H])(c1cnn(C)c1)N1C(=O)c2cc(S(=O)(=O)NC3(C)CC3)ccc2N2C1=NC[C@H]2C#CCN(C)C] | (R)-1-(3-(dimethylamino)prop- 1-yn-1-yl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 56 | 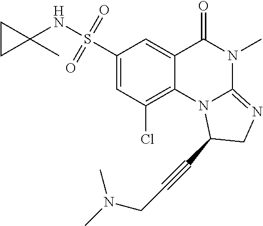 [SMILES: CN(C)CC#C[C@@H]1CN=C2N(C)C(=O)c3cc(S(=O)(=O)NC4(C)CC4)cc(Cl)c3N21] | (R)-9-chloro-1-(3- (dimethylamino)prop-1-yn-1- yl)-4-methyl-N-(1- methylcyclopropyl)-5-oxo- 1,2,4,5-tetrahydroimidazo[1,2- a]quinazoline-7-sulfonamide |
| 57 | 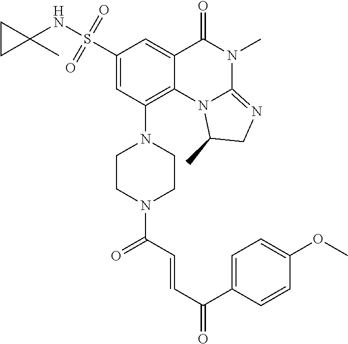 [SMILES: COc1ccc(C(=O)/C=C/C(=O)N2CCN(c3cc(S(=O)(=O)NC4(C)CC4)cc4c3N3C(=NC[C@H]3C)N(C)C4=O)CC2)cc1] | (R,E)-9-(4-(4-(4- methoxyphenyl)-4-oxobut-2- enoyl)piperazin-1-yl)-1,4- dimethyl-N-(1- methylcyclopropyl)-5-oxo- 1,2,4,5-tetrahydroimidazo[1,2- a]quinazoline-7-sulfonamide |
| 58 | 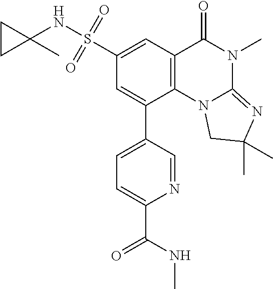 [SMILES: CNC(=O)c1ccc(-c2cc(S(=O)(=O)NC3(C)CC3)cc3c2N2CC(C)(C)N=C2N(C)C3=O)cn1] | N-methyl-5-(2,2,4-trimethyl-7- (N-(1- methylcyclopropyl)sulfamoyl)- 5-oxo-1,2,4,5- tetrahydroimidazo[1,2- a]quinazolin-9-yl)picolinamide |
| 59 | 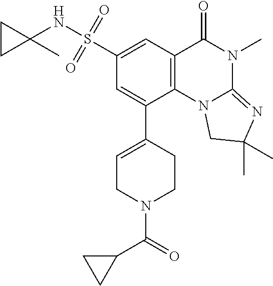 [SMILES: CN1C(=O)c2cc(S(=O)(=O)NC3(C)CC3)cc(C3=CCN(C(=O)C4CC4)CC3)c2N2CC(C)(C)N=C12] | 9-(1-(cyclopropanecarbonyl)- 1,2,3,6-tetrahydropyridin-4-yl)- 2,2,4-trimethyl-N-(1- methylcyclopropyl)-5-oxo- 1,2,4,5-tetrahydroimidazo[1,2- a]quinazoline-7-sulfonamide |
| 60 | 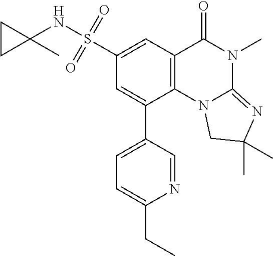 [SMILES: CCc1ccc(-c2cc(S(=O)(=O)NC3(C)CC3)cc3c2N2CC(C)(C)N=C2N(C)C3=O)cn1] | 9-(6-methoxypyridin-3-yl)- 2,2,4-trimethyl-N-(1- methylcyclopropyl)-5-oxo- 1,2,4,5-tetrahydroimidazo[1,2- a]quinazoline-7-sulfonamide |
| 61 | 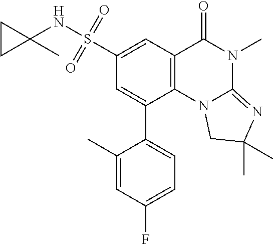 [SMILES: Cc1cc(F)ccc1-c1cc(S(=O)(=O)NC2(C)CC2)cc2c1N1CC(C)(C)N=C1N(C)C2=O] | 9-(4-fluoro-2-methylphenyl)- 2,2,4-trimethyl-N-(1- methylcyclopropyl)-5-oxo- 1,2,4,5-tetrahydroimidazo[1,2- a]quinazoline-7-sulfonamide |
| 62 | 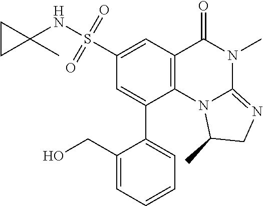 [SMILES: C[C@@H]1CN=C2N(C)C(=O)c3cc(S(=O)(=O)NC4(C)CC4)cc(-c4ccccc4CO)c3N21] | (R)-9-(2- (hydroxymethyl)phenyl)-1,4- dimethyl-N-(1- methylcyclopropyl)-5-oxo- 1,2,4,5-tetrahydroimidazo[1,2- a]quinazoline-7-sulfonamide |
| 63 | 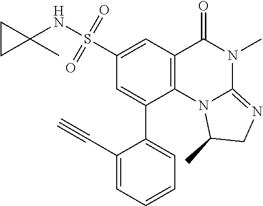 [SMILES: C#Cc1ccccc1-c1cc(S(=O)(=O)NC2(C)CC2)cc2c1N1C(=NC[C@H]1C)N(C)C2=O] | (R)-9-(2-cyanophenyl)-1,4- dimethyl-N-(1- methylcyclopropyl)-5-oxo- 1,2,4,5-tetrahydroimidazo[1,2- a]quinazoline-7-sulfonamide |
| 64 | 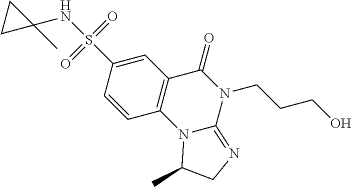 [SMILES: C[C@@H]1CN=C2N(CCCO)C(=O)c3cc(S(=O)(=O)NC4(C)CC4)ccc3N21] | (R)-4-(3-hydroxypropyl)-1- methyl-N-(1- methylcyclopropyl)-5-oxo- 1,2,4,5-tetrahydroimidazo[1,2- a]quinazoline-7-sulfonamide |
| 65 | 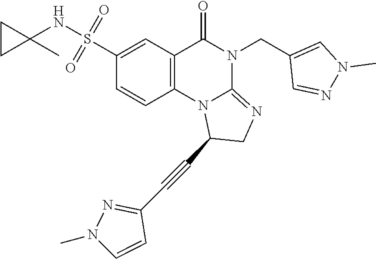 [SMILES: Cn1cc(CN2C(=O)c3cc(S(=O)(=O)NC4(C)CC4)ccc3N3C2=NC[C@H]3C#Cc2ccn(C)n2)cn1] | (R)-1-((1-methyl-1H-pyrazol-3- yl)ethynyl)-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 66 | 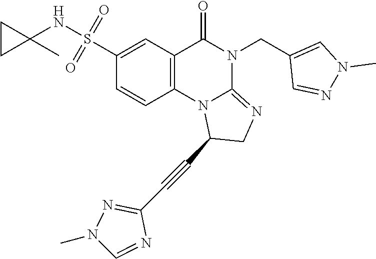 [SMILES: Cn1cc(CN2C(=O)c3cc(S(=O)(=O)NC4(C)CC4)ccc3N3C2=NC[C@H]3C#Cc2ncn(C)n2)cn1] | (R)-1-((1-methyl-1H-1,2,4- triazol-3-yl)ethynyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 67 | 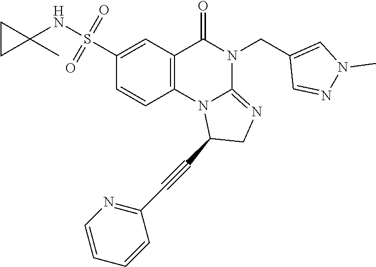 [SMILES: Cn1cc(CN2C(=O)c3cc(S(=O)(=O)NC4(C)CC4)ccc3N3C2=NC[C@H]3C#Cc2ccccn2)cn1] | (R)-4-((1-methyl-1H-pyrazol-4- yl)methyl)-N-(1- methylcyclopropyl)-5-oxo-1- (pyridin-2-ylethynyl)-1,2,4,5- tetrahydroimidazo[1,2- a]quinazoline-7-sulfonamide |
| 68 | 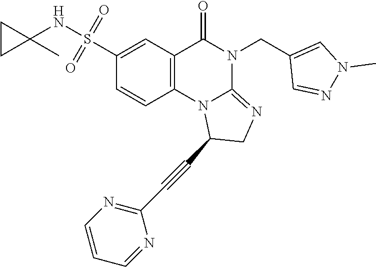 [SMILES: Cn1cc(CN2C(=O)c3cc(S(=O)(=O)NC4(C)CC4)ccc3N3C2=NC[C@H]3C#Cc2ncccn2)cn1] | (R)-4-((1-methyl-1H-pyrazol-4- yl)methyl)-N-(1- methylcyclopropyl)-5-oxo-1- (pyrimidin-2-ylethynyl)- 1,2,4,5-tetrahydroimidazo[1,2- a]quinazoline-7-sulfonamide |
| 69 | 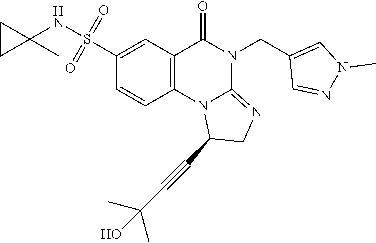 [SMILES: Cn1cc(CN2C(=O)c3cc(S(=O)(=O)NC4(C)CC4)ccc3N3C2=NC[C@H]3C#CC(C)(C)O)cn1] | (R)-1-(3-hydroxy-3-methylbut- 1-yn-1-yl)-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 70 | 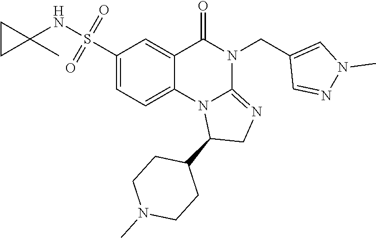 [SMILES: CN1CCC([C@@H]2CN=C3N(Cc4cnn(C)c4)C(=O)c4cc(S(=O)(=O)NC5(C)CC5)ccc4N32)CC1] | (R)-4-((1-methyl-1H-pyrazol-4- yl)methyl)-N-(1- methylcyclopropyl)-1-(1- methylpiperidin-4-yl)-5-oxo- 1,2,4,5-tetrahydroimidazo[1,2- a]quinazoline-7-sulfonamide |
| 71 | 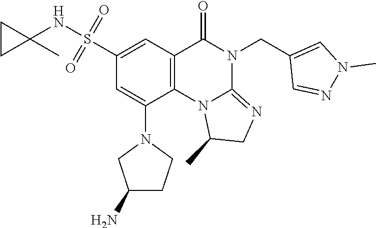 [SMILES: C[C@@H]1CN=C2N(Cc3cnn(C)c3)C(=O)c3cc(S(=O)(=O)NC4(C)CC4)cc(N4CC[C@@H](N)C4)c3N21] | (R)-9-((R)-3-aminopyrrolidin- 1-yl)-1-methyl-4-((1-methyl- 1H-pyrazol-4-yl)methyl)-N-(1- methylcyclopropyl)-5-oxo- 1,2,4,5-tetrahydroimidazo[1,2- a]quinazoline-7-sulfonamide |
| 72 | 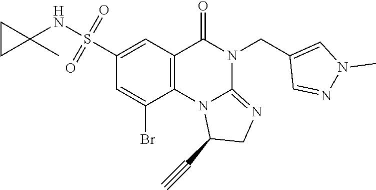 [SMILES: C#C[C@@H]1CN=C2N(Cc3cnn(C)c3)C(=O)c3cc(S(=O)(=O)NC4(C)CC4)cc(Br)c3N21] | (R)-9-bromo-1-ethynyl-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 73 | 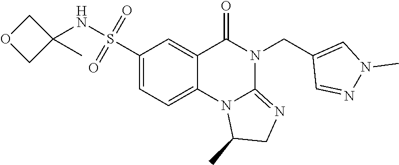 [SMILES: C[C@@H]1CN=C2N(Cc3cnn(C)c3)C(=O)c3cc(S(=O)(=O)NC4(C)COC4)ccc3N21] | (R)-1-methyl-4-((1-methyl-1H- pyrazol-4-yl)methyl)-N-(3- methyloxetan-3-yl)-5-oxo- 1,2,4,5-tetrahydroimidazo[1,2- a]quinazoline-7-sulfonamide |
| 74 | 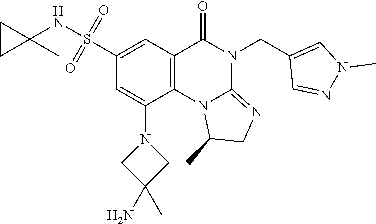 [SMILES: C[C@@H]1CN=C2N(Cc3cnn(C)c3)C(=O)c3cc(S(=O)(=O)NC4(C)CC4)cc(N4CC(C)(N)C4)c3N21] | (R)-9-(3-amino-3- methylazetidin-1-yl)-1-methyl- 4-((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 75 | 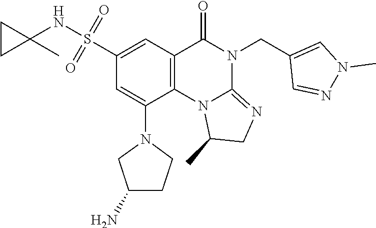 [SMILES: C[C@@H]1CN=C2N(Cc3cnn(C)c3)C(=O)c3cc(S(=O)(=O)NC4(C)CC4)cc(N4CC[C@H](N)C4)c3N21] | (R)-9-((S)-3-aminopyrrolidin- 1-yl)-1-methyl-4-((1-methyl- 1H-pyrazol-4-yl)methyl)-N-(1- methylcyclopropyl)-5-oxo- 1,2,4,5-tetrahydroimidazo[1,2- a]quinazoline-7-sulfonamide |
| 76 | 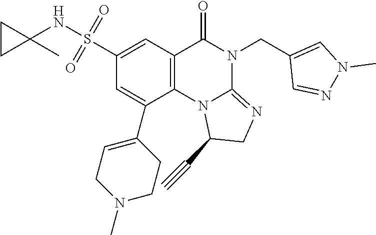 [SMILES: C#C[C@@H]1CN=C2N(Cc3cnn(C)c3)C(=O)c3cc(S(=O)(=O)NC4(C)CC4)cc(C4=CCN(C)CC4)c3N21] | (R)-1-ethynyl-9-(1-methyl- 1,2,3,6-tetrahydropyridin-4-yl)- 4-((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 77 | 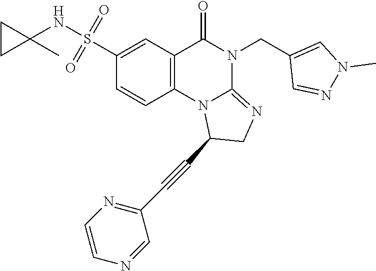 [SMILES: Cn1cc(CN2C(=O)c3cc(S(=O)(=O)NC4(C)CC4)ccc3N3C2=NC[C@H]3C#Cc2cnccn2)cn1] | (R)-4-((1-methyl-1H-pyrazol-4- yl)methyl)-N-(1- methylcyclopropyl)-5-oxo-1- (pyrazin-2-ylethynyl)-1,2,4,5- tetrahydroimidazo[1,2- a]quinazoline-7-sulfonamide |
| 78 | 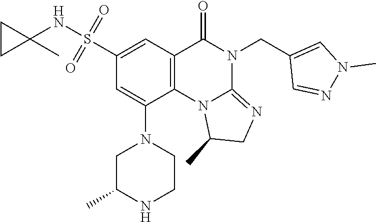 [SMILES: C[C@@H]1CN(c2cc(S(=O)(=O)NC3(C)CC3)cc3c2N2C(=NC[C@H]2C)N(Cc2cnn(C)c2)C3=O)CCN1] | (R)-1-methyl-4-((1-methyl-1H- pyrazol-4-yl)methyl)-N-(1- methylcyclopropyl)-9-((R)-3- methylpiperazin-1-yl)-5-oxo- 1,2,4,5-tetrahydroimidazo[1,2- a]quinazoline-7-sulfonamide |
| 79 | 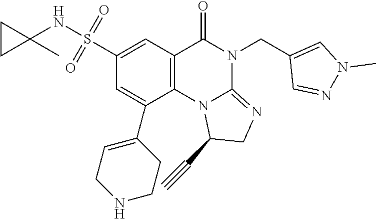 [SMILES: C#C[C@@H]1CN=C2N(Cc3cnn(C)c3)C(=O)c3cc(S(=O)(=O)NC4(C)CC4)cc(C4=CCNCC4)c3N21] | (R)-1-ethynyl-4-((1-methyl-1H- pyrazol-4-yl)methyl)-N-(1- methylcyclopropyl)-5-oxo-9- (1,2,3,6-tetrahydropyridin-4- yl)-1,2,4,5- tetrahydroimidazo[1,2- a]quinazoline-7-sulfonamide |
| 80 | 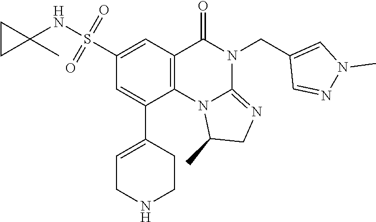 [SMILES: C[C@@H]1CN=C2N(Cc3cnn(C)c3)C(=O)c3cc(S(=O)(=O)NC4(C)CC4)cc(C4=CCNCC4)c3N21] | (R)-1-methyl-4-((1-methyl-1H- pyrazol-4-yl)methyl)-N-(1- methylcyclopropyl)-5-oxo-9- (1,2,3,6-tetrahydropyridin-4- yl)-1,2,4,5- tetrahydroimidazo[1,2- a]quinazoline-7-sulfonamide |
| 81 | 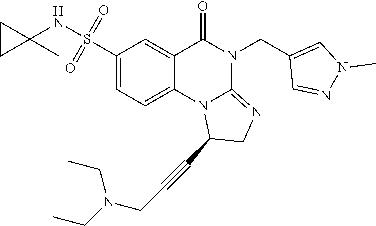 [SMILES: CCN(CC)CC#C[C@@H]1CN=C2N(Cc3cnn(C)c3)C(=O)c3cc(S(=O)(=O)NC4(C)CC4)ccc3N21] | (R)-1-(3-(diethylamino)prop-1- yn-1-yl)-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 82 | 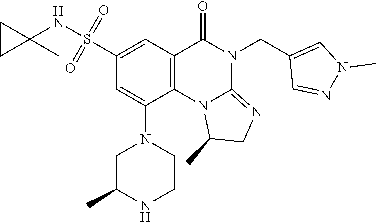 [SMILES: C[C@@H]1CN=C2N(Cc3cnn(C)c3)C(=O)c3cc(S(=O)(=O)NC4(C)CC4)cc(N4CCN[C@@H](C)C4)c3N21] | (R)-1-methyl-4-((1-methyl-1H- pyrazol-4-yl)methyl)-N-(1- methylcyclopropyl)-9-((S)-3- methylpiperazin-1-yl)-5-oxo- 1,2,4,5-tetrahydroimidazo[1,2- a]quinazoline-7-sulfonamide |
| 83 | 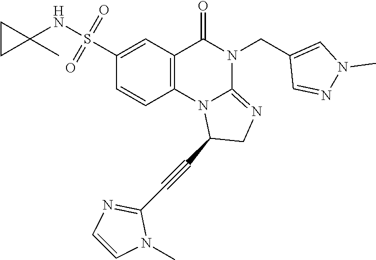 [SMILES: Cn1cc(CN2C(=O)c3cc(S(=O)(=O)NC4(C)CC4)ccc3N3C2=NC[C@H]3C#Cc2nccn2C)cn1] | (R)-1-((1-methyl-1H-imidazol- 2-yl)ethynyl)-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 84 | 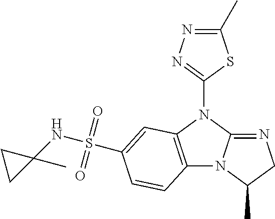 [SMILES: Cc1nnc(N2C3=NC[C@@H](C)N3c3ccc(S(=O)(=O)NC4(C)CC4)cc32)s1] | (R)-3-methyl-9-(5-methyl- 1,3,4-thiadiazol-2-yl)-N-(1- methylcyclopropyl)-2,9- dihydro-3H- benzo[d]imidazo[1,2- a]imidazole-7-sulfonamide |
| 85 | 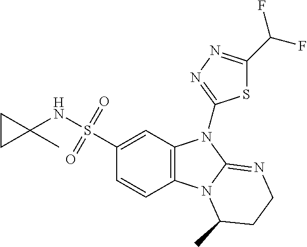 [SMILES: C[C@@H]1CCN=C2N(c3nnc(C(F)F)s3)c3cc(S(=O)(=O)NC4(C)CC4)ccc3N21] | (R)-10-(5-(difluoromethyl)- 1,3,4-thiadiazol-2-yl)-4-methyl- N-(1-methylcyclopropyl)- 2,3,4,10- tetrahydrobenzo[4,5]imidazo[1, 2-a]pyrimidine-8-sulfonamide |
| 86 | 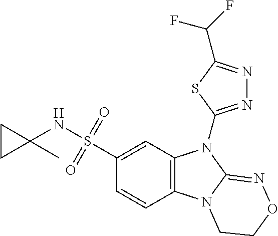 [SMILES: CC1(NS(=O)(=O)c2ccc3c(c2)N(c2nnc(C(F)F)s2)C2=NOCCN23)CC1] | 10-(5-(difluoromethyl)-1,3,4- thiadiazol-2-yl)-N-(1- methylcyclopropyl)-3,4- dihydro-10H- benzo[4,5]imidazo[2,1- c][1,2,4]oxadiazine-8- sulfonamide |
| 87 | 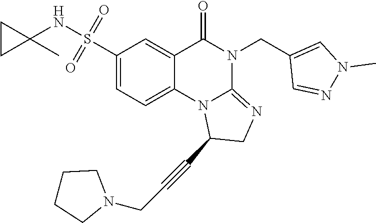 [SMILES: Cn1cc(CN2C(=O)c3cc(S(=O)(=O)NC4(C)CC4)ccc3N3C2=NC[C@H]3C#CCN2CCCC2)cn1] | (R)-4-((1-methyl-1H-pyrazol-4- yl)methyl)-N-(1- methylcyclopropyl)-5-oxo-1- (3-(pyrrolidin-1-yl)prop-1-yn- 1-yl)-1,2,4,5- tetrahydroimidazo[1,2- a]quinazoline-7-sulfonamide |
| 88 | 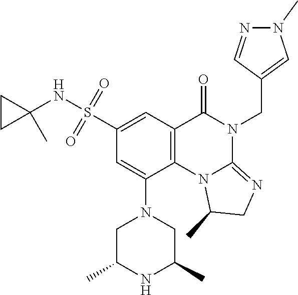 [SMILES: C[C@@H]1CN(c2cc(S(=O)(=O)NC3(C)CC3)cc3c2N2C(=NC[C@H]2C)N(Cc2cnn(C)c2)C3=O)C[C@@H](C)N1] | (R)-9-((3R,5R)-3,5- dimethylpiperazin-1-yl)-1- methyl-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 89 | 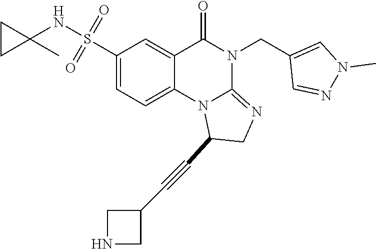 [SMILES: Cn1cc(CN2C(=O)c3cc(S(=O)(=O)NC4(C)CC4)ccc3N3C2=NC[C@H]3C#CC2CNC2)cn1] | (R)-1-(azetidin-3-ylethynyl)-4- ((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 90 | 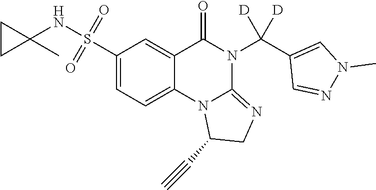 [SMILES: [2H]C([2H])(c1cnn(C)c1)N1C(=O)c2cc(S(=O)(=O)NC3(C)CC3)ccc2N2C1=NC[C@@H]2C#C] | (S)-1-ethynyl-4-((1-methyl-1H- pyrazol-4-yl)methyl-d₂)-N-(1- methylcyclopropyl)-5-oxo- 1,2,4,5-tetrahydroimidazo[1,2- a]quinazoline-7-sulfonamide |
| 91 | 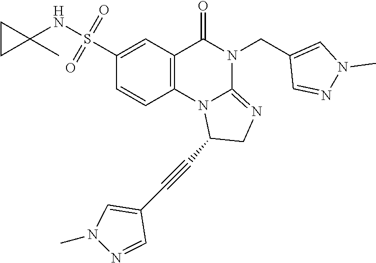 [SMILES: Cn1cc(C#C[C@H]2CN=C3N(Cc4cnn(C)c4)C(=O)c4cc(S(=O)(=O)NC5(C)CC5)ccc4N32)cn1] | (S)-1-((1-methyl-1H-pyrazol-4- yl)ethynyl)-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 92 | 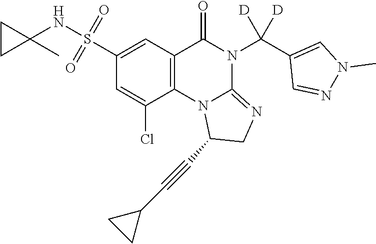 [SMILES: [2H]C([2H])(c1cnn(C)c1)N1C(=O)c2cc(S(=O)(=O)NC3(C)CC3)cc(Cl)c2N2C1=NC[C@@H]2C#CC1CC1] | (S)-9-chloro-1- (cyclopropylethynyl)-4-((1- methyl-1H-pyrazol-4- yl)methyl-d₂)-N-(1- methylcyclopropyl)-5-oxo- 1,2,4,5-tetrahydroimidazo[1,2- a]quinazoline-7-sulfonamide |
| 93 | 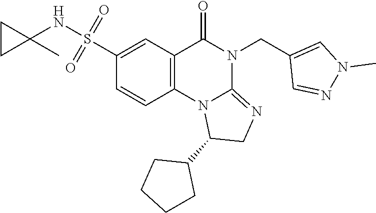 [SMILES: Cn1cc(CN2C(=O)c3cc(S(=O)(=O)NC4(C)CC4)ccc3N3C2=NC[C@@H]3C2CCCC2)cn1] | (S)-1-cyclopentyl-4-((1-methyl- 1H-pyrazol-4-yl)methyl)-N-(1- methylcyclopropyl)-5-oxo- 1,2,4,5-tetrahydroimidazo[1,2- a]quinazoline-7-sulfonamide |
| 94 | 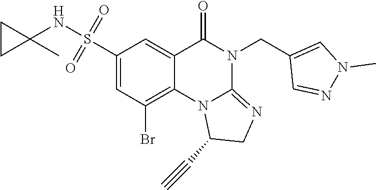 [SMILES: C#C[C@H]1CN=C2N(Cc3cnn(C)c3)C(=O)c3cc(S(=O)(=O)NC4(C)CC4)cc(Br)c3N21] | (S)-9-bromo-1-ethynyl-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 95 | 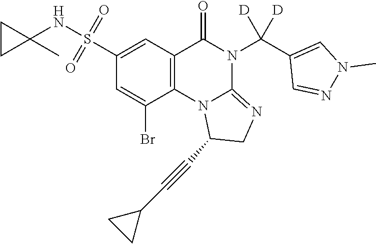 [SMILES: [2H]C([2H])(c1cnn(C)c1)N1C(=O)c2cc(S(=O)(=O)NC3(C)CC3)cc(Br)c2N2C1=NC[C@@H]2C#CC1CC1] | (S)-9-bromo-1- (cyclopropylethynyl)-4-((1- methyl-1H-pyrazol-4- yl)methyl-d₂)-N-(1- methylcyclopropyl)-5-oxo- 1,2,4,5-tetrahydroimidazo[1,2- a]quinazoline-7-sulfonamide |
| 96 | 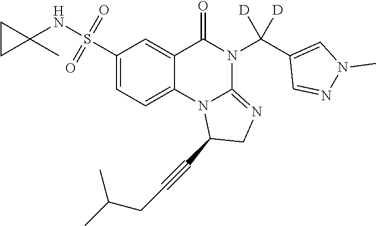 [SMILES: [2H]C([2H])(c1cnn(C)c1)N1C(=O)c2cc(S(=O)(=O)NC3(C)CC3)ccc2N2C1=NC[C@H]2C#CCC(C)C] | (R)-4-((1-methyl-1H-pyrazol-4- yl)methyl-d2)-N-(1- methylcyclopropyl)-1-(4- methylpent-1-yn-1-yl)-5-oxo- 1,2,4,5-tetrahydroimidazo[1,2- a]quinazoline-7-sulfonamide |
| 97 | 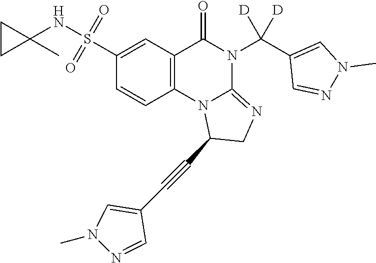 [SMILES: [2H]C([2H])(c1cnn(C)c1)N1C(=O)c2cc(S(=O)(=O)NC3(C)CC3)ccc2N2C1=NC[C@H]2C#Cc1cnn(C)c1] | (R)-1-((1-methyl-1H-pyrazol-4- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 98 | 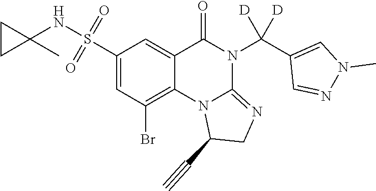 [SMILES: [2H]C([2H])(c1cnn(C)c1)N1C(=O)c2cc(S(=O)(=O)NC3(C)CC3)cc(Br)c2N2C1=NC[C@H]2C#C] | (R)-9-bromo-1-ethynyl-4-((1- methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 99 | 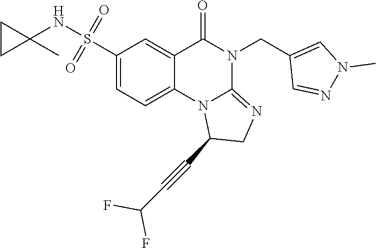 [SMILES: Cn1cc(CN2C(=O)c3cc(S(=O)(=O)NC4(C)CC4)ccc3N3C2=NC[C@H]3C#CC(F)F)cn1] | (R)-1-(3,3-difluoroprop-1-yn-1- yl)-4-((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 100 | 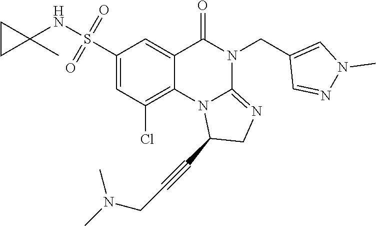 [SMILES: CN(C)CC#C[C@@H]1CN=C2N(Cc3cnn(C)c3)C(=O)c3cc(S(=O)(=O)NC4(C)CC4)cc(Cl)c3N21] | (R)-9-chloro-1-(3- (dimethylamino)prop-1-yn-1- yl)-4-((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 101 | 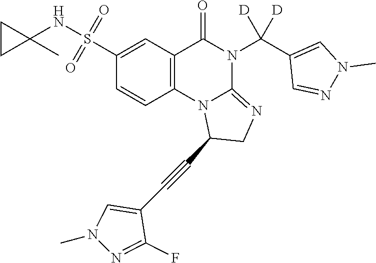 [SMILES: [2H]C([2H])(c1cnn(C)c1)N1C(=O)c2cc(S(=O)(=O)NC3(C)CC3)ccc2N2C1=NC[C@H]2C#Cc1cn(C)nc1F] | (R)-1-((3-fluoro-1-methyl-1H- pyrazol-4-yl)ethynyl)-4-((1- methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 102 | 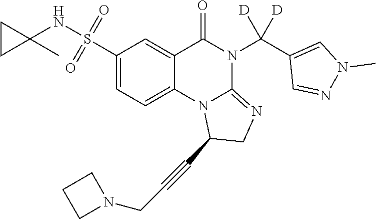 [SMILES: [2H]C([2H])(c1cnn(C)c1)N1C(=O)c2cc(S(=O)(=O)NC3(C)CC3)ccc2N2C1=NC[C@H]2C#CCN1CCC1] | (R)-1-(3-(azetidin-1-yl)prop-1- yn-1-yl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 103 | 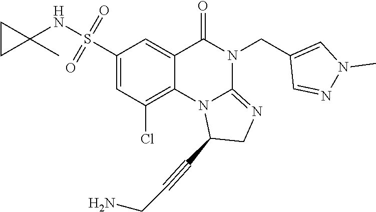 [SMILES: Cn1cc(CN2C(=O)c3cc(S(=O)(=O)NC4(C)CC4)cc(Cl)c3N3C2=NC[C@H]3C#CCN)cn1] | (R)-1-(3-aminoprop-1-yn-1-yl)- 9-chloro-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 104 | 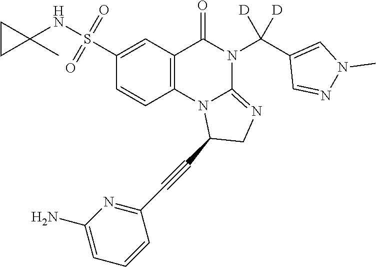 [SMILES: [2H]C([2H])(c1cnn(C)c1)N1C(=O)c2cc(S(=O)(=O)NC3(C)CC3)ccc2N2C1=NC[C@H]2C#Cc1cccc(N)n1] | (R)-1-((6-aminopyridin-2- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 105 | 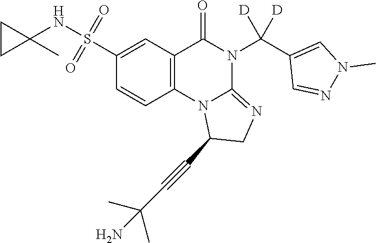 [SMILES: [2H]C([2H])(c1cnn(C)c1)N1C(=O)c2cc(S(=O)(=O)NC3(C)CC3)ccc2N2C1=NC[C@H]2C#CC(C)(C)N] | (R)-1-(3-amino-3-methylbut-1- yn-1-yl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 106 | 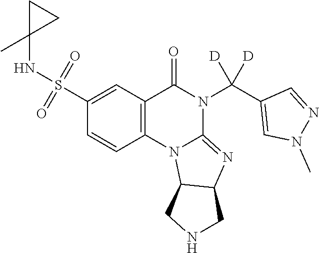 [SMILES: [2H]C([2H])(c1cnn(C)c1)N1C(=O)c2cc(S(=O)(=O)NC3(C)CC3)ccc2N2C1=N[C@H]1CNC[C@H]12] | (7aS,10aR)-6-((1-methyl-1H- pyrazol-4-yl)methyl-d2)-N-(1- methylcyclopropyl)-5-oxo- 6,7a,8,9,10,10a-hexahydro-5H- pyrrolo[3′,4′:4,5]imidazo[1,2- a]quinazoline-3-sulfonamide |
| 107 | 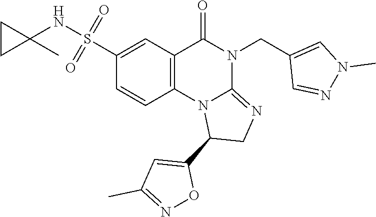 [SMILES: Cc1cc([C@@H]2CN=C3N(Cc4cnn(C)c4)C(=O)c4cc(S(=O)(=O)NC5(C)CC5)ccc4N32)on1] | (S)-4-((1-methyl-1H-pyrazol-4- yl)methyl)-N-(1- methylcyclopropyl)-1-(3- methylisoxazol-5-yl)-5-oxo- 1,2,4,5-tetrahydroimidazo[1,2- a]quinazoline-7-sulfonamide |
| 108 | 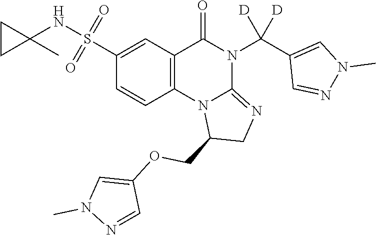 [SMILES: [2H]C([2H])(c1cnn(C)c1)N1C(=O)c2cc(S(=O)(=O)NC3(C)CC3)ccc2N2C1=NC[C@H]2COc1cnn(C)c1] | (S)-4-((1-methyl-1H-pyrazol-4- yl)methyl-d2)-1-(((1-methyl- 1H-pyrazol-4-yl)oxy)methyl)- N-(1-methylcyclopropyl)-5- oxo-1,2,4,5- tetrahydroimidazo[1,2- a]quinazoline-7-sulfonamide |
| 109 | 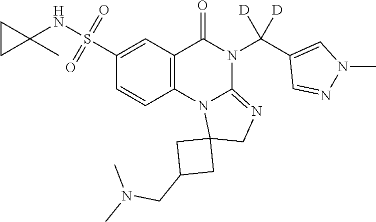 [SMILES: [2H]C([2H])(c1cnn(C)c1)N1C(=O)c2cc(S(=O)(=O)NC3(C)CC3)ccc2N2C1=NCC21CC(CN(C)C)C1] | 3-((dimethylamino)methyl)-4′- ((1-methyl-1H-pyrazol-4- yl)methyl-d2)-N-(1- methylcyclopropyl)-5′-oxo- 4′,5′-dihydro-2′H- spiro[cyclobutane-1,1′- imidazo[1,2-a]quinazoline]-7′- sulfonamide |
| 110 | 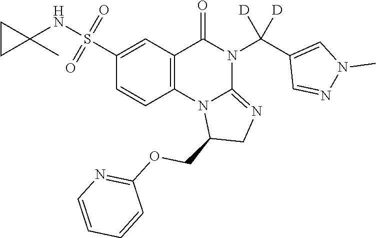 [SMILES: [2H]C([2H])(c1cnn(C)c1)N1C(=O)c2cc(S(=O)(=O)NC3(C)CC3)ccc2N2C1=NC[C@H]2COc1ccccn1] | (S)-4-((1-methyl-1H-pyrazol-4- yl)methyl-d2)-N-(1- methylcyclopropyl)-5-oxo-1- ((pyridin-2-yloxy)methyl)- 1,2,4,5-tetrahydroimidazo[1,2- a]quinazoline-7-sulfonamide |
| 111 | 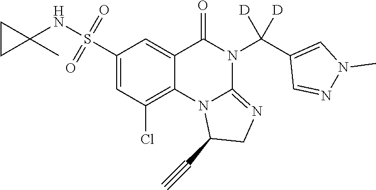 [SMILES: [2H]C([2H])(c1cnn(C)c1)N1C(=O)c2cc(S(=O)(=O)NC3(C)CC3)cc(Cl)c2N2C1=NC[C@H]2C#C] | (R)-9-chloro-1-ethynyl-4-((1- methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 112 | 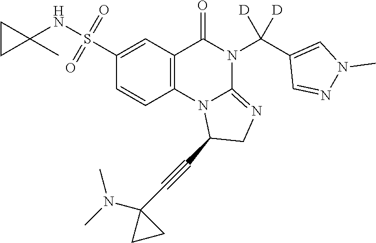 [SMILES: [2H]C([2H])(c1cnn(C)c1)N1C(=O)c2cc(S(=O)(=O)NC3(C)CC3)ccc2N2C1=NC[C@H]2C#CC1(N(C)C)CC1] | (R)-1-((1- (dimethylamino)cyclopropyl) ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 113 | 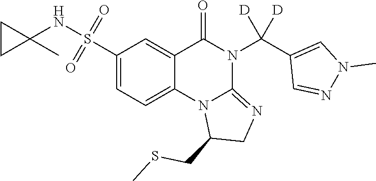 [SMILES: [2H]C([2H])(c1cnn(C)c1)N1C(=O)c2cc(S(=O)(=O)NC3(C)CC3)ccc2N2C1=NC[C@H]2CSC] | (S)-4-((1-methyl-1H-pyrazol-4- yl)methyl-d2)-N-(1- methylcyclopropyl)-1- ((methylthio)methyl)-5-oxo- 1,2,4,5-tetrahydroimidazo[1,2- a]quinazoline-7-sulfonamide |
| 114 | 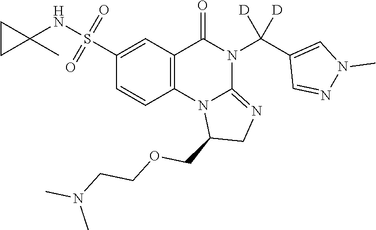 [SMILES: [2H]C([2H])(c1cnn(C)c1)N1C(=O)c2cc(S(=O)(=O)NC3(C)CC3)ccc2N2C1=NC[C@H]2COCCN(C)C] | (S)-1-((2- (dimethylamino)ethoxy)methyl)- 4-((1-methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 115 | 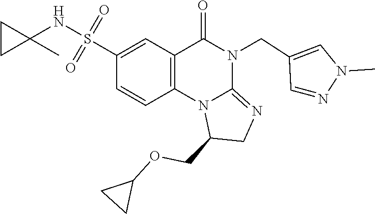 [SMILES: Cn1cc(CN2C(=O)c3cc(S(=O)(=O)NC4(C)CC4)ccc3N3C2=NC[C@H]3COC2CC2)cn1] | (S)-1-(cyclopropoxymethyl)-4- ((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 116 | 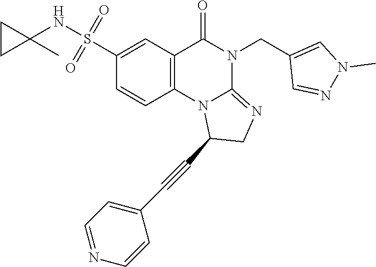 [SMILES: Cn1cc(CN2C(=O)c3cc(S(=O)(=O)NC4(C)CC4)ccc3N3C2=NC[C@H]3C#Cc2ccncc2)cn1] | (R)-4-((1-methyl-1H-pyrazol-4- yl)methyl)-N-(1- methylcyclopropyl)-5-oxo-1- (pyridin-4-ylethynyl)-1,2,4,5- tetrahydroimidazo[1,2- a]quinazoline-7-sulfonamide |
| 117 | 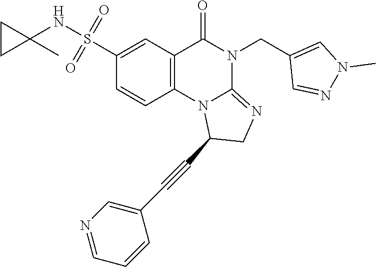 [SMILES: Cn1cc(CN2C(=O)c3cc(S(=O)(=O)NC4(C)CC4)ccc3N3C2=NC[C@H]3C#Cc2cccnc2)cn1] | (R)-4-((1-methyl-1H-pyrazol-4- yl)methyl)-N-(1- methylcyclopropyl)-5-oxo-1- (pyridin-3-ylethynyl)-1,2,4,5- tetrahydroimidazo[1,2- a]quinazoline-7-sulfonamide |
| 118 | 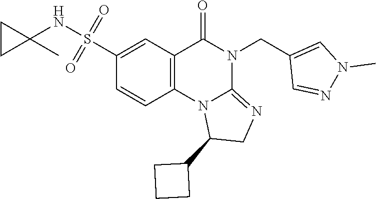 [SMILES: Cn1cc(CN2C(=O)c3cc(S(=O)(=O)NC4(C)CC4)ccc3N3C2=NC[C@H]3C2CCC2)cn1] | (R)-1-cyclobutyl-4-((1-methyl- 1H-pyrazol-4-yl)methyl)-N-(1- methylcyclopropyl)-5-oxo- 1,2,4,5-tetrahydroimidazo[1,2- a]quinazoline-7-sulfonamide |
| 119 | 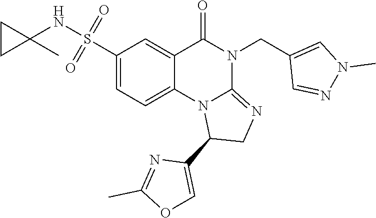 [SMILES: Cc1nc([C@@H]2CN=C3N(Cc4cnn(C)c4)C(=O)c4cc(S(=O)(=O)NC5(C)CC5)ccc4N32)co1] | (S)-4-((1-methyl-1H-pyrazol-4- yl)methyl)-N-(1- methylcyclopropyl)-1-(2- methyloxazol-4-yl)-5-oxo- 1,2,4,5-tetrahydroimidazo[1,2- a]quinazoline-7-sulfonamide |
| 120 | 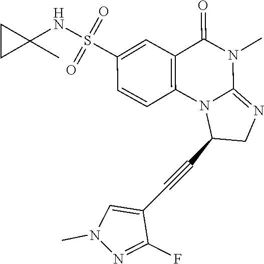 [SMILES: CN1C(=O)c2cc(S(=O)(=O)NC3(C)CC3)ccc2N2C1=NC[C@H]2C#Cc1cn(C)nc1F] | (R)-1-((3-fluoro-1-methyl-1H- pyrazol-4-yl)ethynyl)-4- methyl-N-(1- methylcyclopropyl)-5-oxo- 1,2,4,5-tetrahydroimidazo[1,2- a]quinazoline-7-sulfonamide |
| 121 | 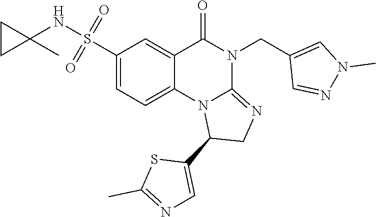 [SMILES: Cc1ncc([C@@H]2CN=C3N(Cc4cnn(C)c4)C(=O)c4cc(S(=O)(=O)NC5(C)CC5)ccc4N32)s1] | (S)-4-((1-methyl-1H-pyrazol-4- yl)methyl)-N-(1- methylcyclopropyl)-1-(2- methylthiazol-5-yl)-5-oxo- 1,2,4,5-tetrahydroimidazo[1,2- a]quinazoline-7-sulfonamide |
| 122 | 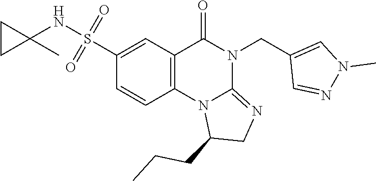 [SMILES: CCC[C@@H]1CN=C2N(Cc3cnn(C)c3)C(=O)c3cc(S(=O)(=O)NC4(C)CC4)ccc3N21] | (R)-4-((1-methyl-1H-pyrazol-4- yl)methyl)-N-(1- methylcyclopropyl)-5-oxo-1- propyl-1,2,4,5- tetrahydroimidazo[1,2- a]quinazoline-7-sulfonamide |
| 123 | 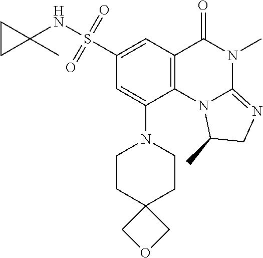 [SMILES: C[C@@H]1CN=C2N(C)C(=O)c3cc(S(=O)(=O)NC4(C)CC4)cc(N4CCC5(CC4)COC5)c3N21] | (R)-1,4-dimethyl-N-(1- methylcyclopropyl)-5-oxo-9- (2-oxa-7-azaspiro[3.5]nonan-7- yl)-1,2,4,5- tetrahydroimidazo[1,2- a]quinazoline-7-sulfonamide |
| 124 | 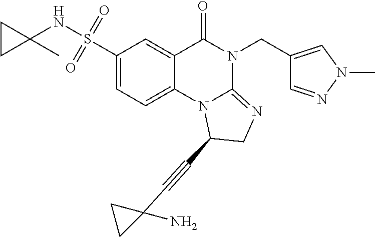 [SMILES: Cn1cc(CN2C(=O)c3cc(S(=O)(=O)NC4(C)CC4)ccc3N3C2=NC[C@H]3C#CC2(N)CC2)cn1] | (R)-1-((1- aminocyclopropyl)ethynyl)-4- ((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 125 | 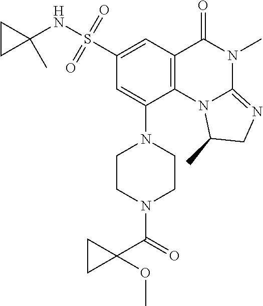 [SMILES: COC1(C(=O)N2CCN(c3cc(S(=O)(=O)NC4(C)CC4)cc4c3N3C(=NC[C@H]3C)N(C)C4=O)CC2)CC1] | (R)-9-(4-(1- methoxycyclopropane-1- carbonyl)piperazin-1-yl)-1,4- dimethyl-N-(1- methylcyclopropyl)-5-oxo- 1,2,4,5-tetrahydroimidazo[1,2- a]quinazoline-7-sulfonamide |
| 126 | 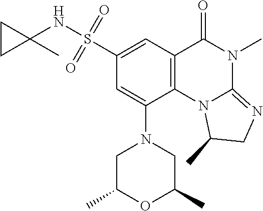 [SMILES: C[C@@H]1CN(c2cc(S(=O)(=O)NC3(C)CC3)cc3c2N2C(=NC[C@H]2C)N(C)C3=O)C[C@@H](C)O1] | (R)-9-((2R,6R)-2,6- dimethylmorpholino)-1,4- dimethyl-N-(1- methylcyclopropyl)-5-oxo- 1,2,4,5-tetrahydroimidazo[1,2- a]quinazoline-7-sulfonamide |
| 127 | 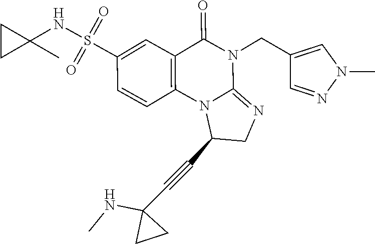 [SMILES: CNC1(C#C[C@@H]2CN=C3N(Cc4cnn(C)c4)C(=O)c4cc(S(=O)(=O)NC5(C)CC5)ccc4N32)CC1] | (R)-4-((1-methyl-1H-pyrazol-4- yl)methyl)-1-((1- (methylamino)cyclopropyl)ethynyl)- N-(1-methylcyclopropyl)- 5-oxo-1,2,4,5- tetrahydroimidazo[1,2- a]quinazoline-7-sulfonamide |
| 128 | 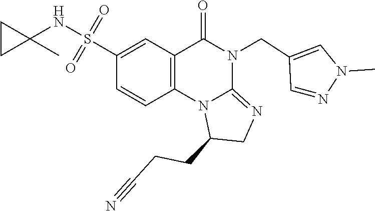 [SMILES: Cn1cc(CN2C(=O)c3cc(S(=O)(=O)NC4(C)CC4)ccc3N3C2=NC[C@H]3CCC#N)cn1] | (R)-1-(2-cyanoethyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 129 | 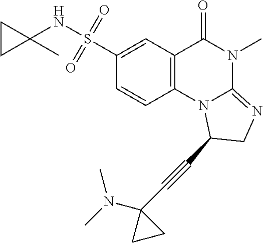 [SMILES: CN1C(=O)c2cc(S(=O)(=O)NC3(C)CC3)ccc2N2C1=NC[C@H]2C#CC1(N(C)C)CC1] | (R)-1-((1- (dimethylamino)cyclopropyl) ethynyl)-4-methyl-N-(1- methylcyclopropyl)-5-oxo- 1,2,4,5-tetrahydroimidazo[1,2- a]quinazoline-7-sulfonamide |
| 130 | 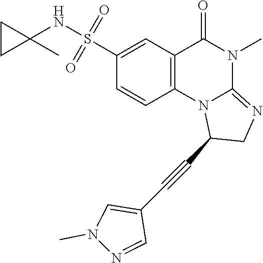 [SMILES: CN1C(=O)c2cc(S(=O)(=O)NC3(C)CC3)ccc2N2C1=NC[C@H]2C#Cc1cnn(C)c1] | (R)-4-methyl-1-((1-methyl-1H- pyrazol-4-yl)ethynyl)-N-(1- methylcyclopropyl)-5-oxo- 1,2,4,5-tetrahydroimidazo[1,2- a]quinazoline-7-sulfonamide |
| 131 | 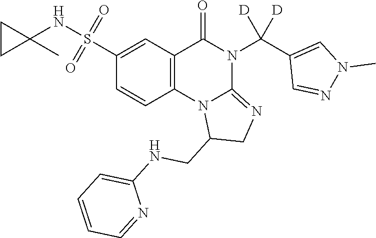 [SMILES: [2H]C([2H])(c1cnn(C)c1)N1C(=O)c2cc(S(=O)(=O)NC3(C)CC3)ccc2N2C1=NCC2CNc1ccccn1] | 4-((1-methyl-1H-pyrazol-4- yl)methyl-d2)-N-(1- methylcyclopropyl)-5-oxo-1- ((pyridin-2-ylamino)methyl)- 1,2,4,5-tetrahydroimidazo[1,2- a]quinazoline-7-sulfonamide |
| 132 | 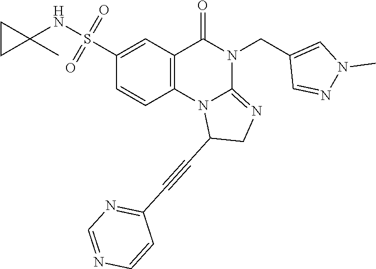 [SMILES: Cn1cc(CN2C(=O)c3cc(S(=O)(=O)NC4(C)CC4)ccc3N3C2=NCC3C#Cc2ccncn2)cn1] | 4-((1-methyl-1H-pyrazol-4- yl)methyl)-N-(1- methylcyclopropyl)-5-oxo-1- (pyrimidin-4-ylethynyl)- 1,2,4,5-tetrahydroimidazo[1,2- a]quinazoline-7-sulfonamide |
| 133 | 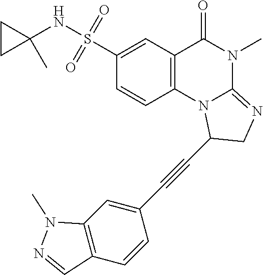 [SMILES: CN1C(=O)c2cc(S(=O)(=O)NC3(C)CC3)ccc2N2C1=NCC2C#Cc1ccc2cnn(C)c2c1] | 4-methyl-1-((1-methyl-1H- indazol-6-yl)ethynyl)-N-(1- methylcyclopropyl)-5-oxo- 1,2,4,5-tetrahydroimidazo[1,2- a]quinazoline-7-sulfonamide |
| 134 | 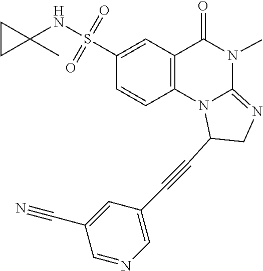 [SMILES: CN1C(=O)c2cc(S(=O)(=O)NC3(C)CC3)ccc2N2C1=NCC2C#Cc1cncc(C#N)c1] | 1-((5-cyanopyridin-3- yl)ethynyl)-4-methyl-N-(1- methylcyclopropyl)-5-oxo- 1,2,4,5-tetrahydroimidazo[1,2- a]quinazoline-7-sulfonamide |
| 135 | 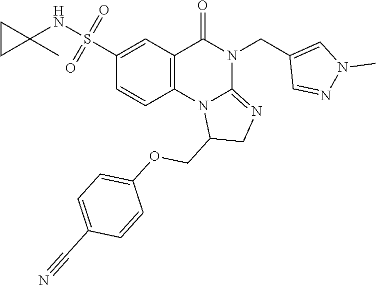 [SMILES: Cn1cc(CN2C(=O)c3cc(S(=O)(=O)NC4(C)CC4)ccc3N3C2=NCC3COc2ccc(C#N)cc2)cn1] | 1-((4-cyanophenoxy)methyl)-4- ((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 136 | 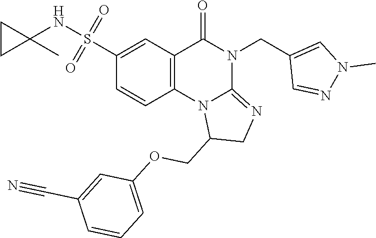 [SMILES: Cn1cc(CN2C(=O)c3cc(S(=O)(=O)NC4(C)CC4)ccc3N3C2=NCC3COc2cccc(C#N)c2)cn1] | 1-((3-cyanophenoxy)methyl)-4- ((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 137 | 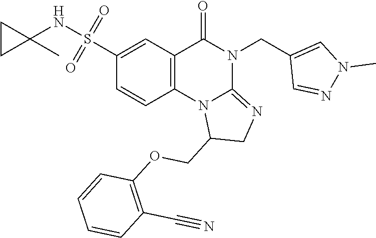 [SMILES: Cn1cc(CN2C(=O)c3cc(S(=O)(=O)NC4(C)CC4)ccc3N3C2=NCC3COc2ccccc2C#N)cn1] | 1-((2-cyanophenoxy)methyl)-4- ((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 138 | 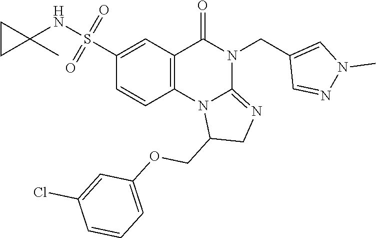 [SMILES: Cn1cc(CN2C(=O)c3cc(S(=O)(=O)NC4(C)CC4)ccc3N3C2=NCC3COc2cccc(Cl)c2)cn1] | 1-((3-chlorophenoxy)methyl)- 4-((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 139 | 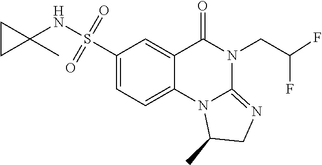 [SMILES: C[C@@H]1CN=C2N(CC(F)F)C(=O)c3cc(S(=O)(=O)NC4(C)CC4)ccc3N21] | (R)-4-(2,2-difluoroethyl)-1- methyl-N-(1- methylcyclopropyl)-5-oxo- 1,2,4,5-tetrahydroimidazo[1,2- a]quinazoline-7-sulfonamide |
| 140 | 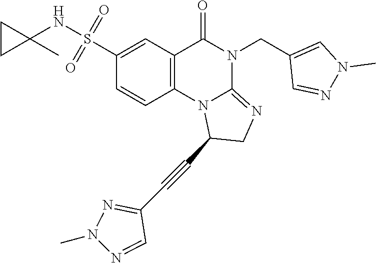 [SMILES: Cn1cc(CN2C(=O)c3cc(S(=O)(=O)NC4(C)CC4)ccc3N3C2=NC[C@H]3C#Cc2cnn(C)n2)cn1] | (R)-4-((1-methyl-1H-pyrazol-4- yl)methyl)-1-((2-methyl-2H- 1,2,3-triazol-4-yl)ethynyl)-N- (1-methylcyclopropyl)-5-oxo- 1,2,4,5-tetrahydroimidazo[1,2- a]quinazoline-7-sulfonamide |
| 141 | 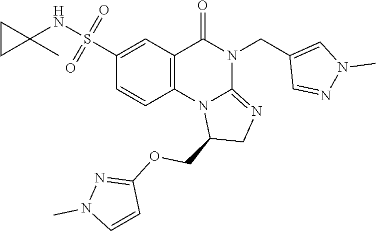 [SMILES: Cn1cc(CN2C(=O)c3cc(S(=O)(=O)NC4(C)CC4)ccc3N3C2=NC[C@H]3COc2ccn(C)n2)cn1] | (S)-1-(((1-methyl-1H-pyrazol- 3-yl)oxy)methyl)-4-((1-methyl- 1H-pyrazol-4-yl)methyl)-N-(1- methylcyclopropyl)-5-oxo- 1,2,4,5-tetrahydroimidazo[1,2- a]quinazoline-7-sulfonamide |
| 142 | 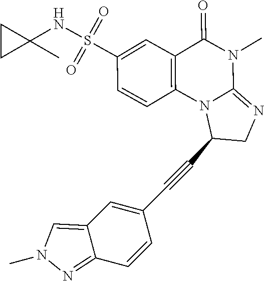 [SMILES: CN1C(=O)c2cc(S(=O)(=O)NC3(C)CC3)ccc2N2C1=NC[C@H]2C#Cc1ccc2nn(C)cc2c1] | (R)-4-methyl-1-((2-methyl-2H- indazol-5-yl)ethynyl)-N-(1- methylcyclopropyl)-5-oxo- 1,2,4,5-tetrahydroimidazo[1,2- a]quinazoline-7-sulfonamide |
| 143 | 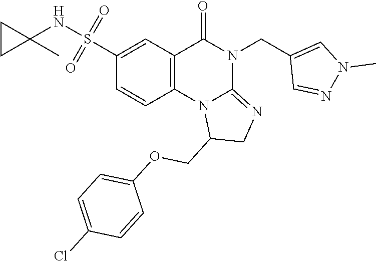 [SMILES: Cn1cc(CN2C(=O)c3cc(S(=O)(=O)NC4(C)CC4)ccc3N3C2=NCC3COc2ccc(Cl)cc2)cn1] | 1-((4-chlorophenoxy)methyl)- 4-((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 144 | 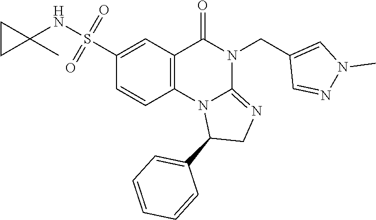 [SMILES: Cn1cc(CN2C(=O)c3cc(S(=O)(=O)NC4(C)CC4)ccc3N3C2=NC[C@H]3c2ccccc2)cn1] | (R)-4-((1-methyl-1H-pyrazol- yl)methyl)-N-(1- methylcyclopropyl)-5-oxo-1- phenyl-1,2,4,5- tetrahydroimidazo[1,2- a]quinazoline-7-sulfonamide |
| 145 | 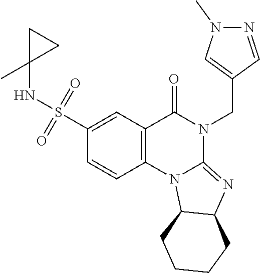 [SMILES: Cn1cc(CN2C(=O)c3cc(S(=O)(=O)NC4(C)CC4)ccc3N3C2=N[C@H]2CCCC[C@H]23)cn1] | (7aS,11aR)-6-((1-methyl-1H- pyrazol-4-yl)methyl)-N-(1- methylcyclopropyl)-5-oxo- 5,6,7a,8,9,10,11,11a- octahydrobenzo[4,5]imidazo[1, 2-a]quinazoline-3-sulfonamide |
| 146 | 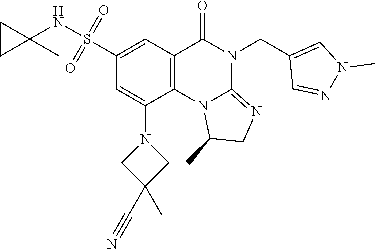 [SMILES: C[C@@H]1CN=C2N(Cc3cnn(C)c3)C(=O)c3cc(S(=O)(=O)NC4(C)CC4)cc(N4CC(C)(C#N)C4)c3N21] | (R)-9-(3-cyano-3- methylazetidin-1-yl)-1-methyl- 4-((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 147 | 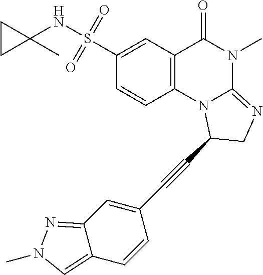 [SMILES: CN1C(=O)c2cc(S(=O)(=O)NC3(C)CC3)ccc2N2C1=NC[C@H]2C#Cc1ccc2cn(C)nc2c1] | (R)-4-methyl-1-((2-methyl-2H- indazol-6-yl)ethynyl)-N-(1- methylcyclopropyl)-5-oxo- 1,2,4,5-tetrahydroimidazo[1,2- a]quinazoline-7-sulfonamide |
| 148 | 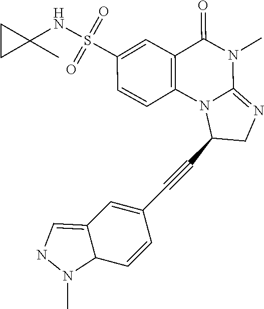 [SMILES: CN1C(=O)c2cc(S(=O)(=O)NC3(C)CC3)ccc2N2C1=NC[C@H]2C#CC1=CC2=CNN(C)C2C=C1] | (R)-4-methyl-1-((1-methyl-1H- indazol-5-yl)ethynyl)-N-(1- methylcyclopropyl)-5-oxo- 1,2,4,5-tetrahydroimidazo[1,2- a]quinazoline-7-sulfonamide |
| 149 | 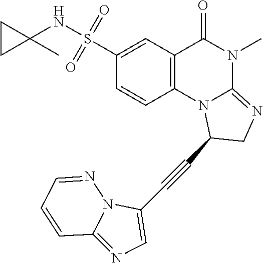 [SMILES: CN1C(=O)c2cc(S(=O)(=O)NC3(C)CC3)ccc2N2C1=NC[C@H]2C#Cc1cnc2cccnn12] | (R)-1-(imidazo[1,2- b]pyridazin-3-ylethynyl)-4- methyl-N-(1- methylcyclopropyl)-5-oxo- 1,2,4,5-tetrahydroimidazo[1,2- a]quinazoline-7-sulfonamide |
| 150 | 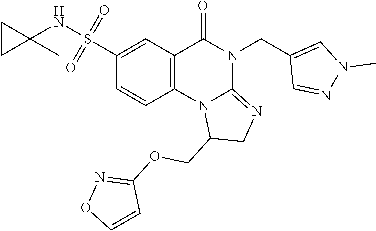 [SMILES: Cn1cc(CN2C(=O)c3cc(S(=O)(=O)NC4(C)CC4)ccc3N3C2=NCC3COc2ccon2)cn1] | 1-((isoxazol-3-yloxy)methyl)- 4-((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 151 | 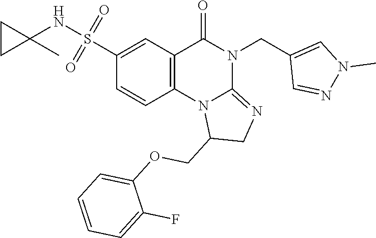 [SMILES: Cn1cc(CN2C(=O)c3cc(S(=O)(=O)NC4(C)CC4)ccc3N3C2=NCC3COc2ccccc2F)cn1] | 1-((2-fluorophenoxy)methyl)-4- ((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 152 | 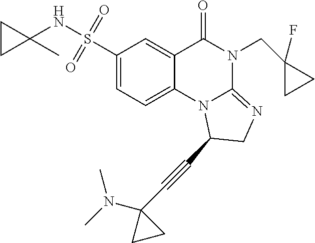 [SMILES: CN(C)C1(C#C[C@@H]2CN=C3N(CC4(F)CC4)C(=O)c4cc(S(=O)(=O)NC5(C)CC5)ccc4N32)CC1] | (R)-1-((1- (dimethylamino)cyclopropyl) ethynyl)-4-((1- fluorocyclopropyl)methyl)-N- (1-methylcyclopropyl)-5-oxo- 1,2,4,5-tetrahydroimidazo[1,2- a]quinazoline-7-sulfonamide |
| 153 | 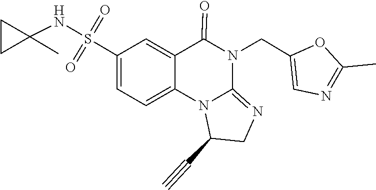 [SMILES: C#C[C@@H]1CN=C2N(Cc3cnc(C)o3)C(=O)c3cc(S(=O)(=O)NC4(C)CC4)ccc3N21] | (R)-1-ethynyl-N-(1- methylcyclopropyl)-4-((2- methyloxazol-5-yl)methyl)-5- oxo-1,2,4,5- tetrahydroimidazo[1,2- a]quinazoline-7-sulfonamide |
| 154 | 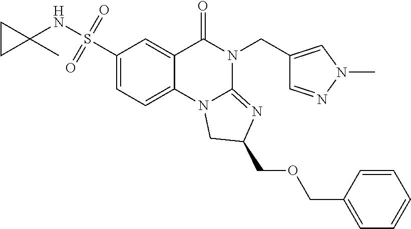 [SMILES: Cn1cc(CN2C(=O)c3cc(S(=O)(=O)NC4(C)CC4)ccc3N3C[C@H](COCc4ccccc4)N=C23)cn1] | (R)-2-((benzyloxy)methyl)-4- ((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 155 | 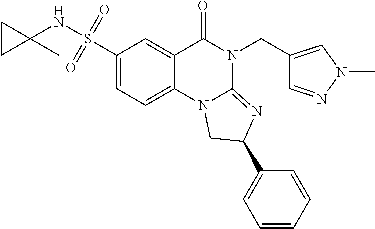 [SMILES: Cn1cc(CN2C(=O)c3cc(S(=O)(=O)NC4(C)CC4)ccc3N3C[C@H](c4ccccc4)N=C23)cn1] | (S)-4-((1-methyl-1H-pyrazol-4- yl)methyl)-N-(1- methylcyclopropyl)-5-oxo-2- phenyl-1,2,4,5- tetrahydroimidazo[1,2- a]quinazoline-7-sulfonamide |
| 156 | 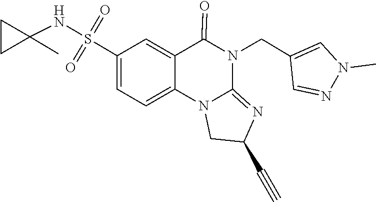 [SMILES: C#C[C@H]1CN2C(=N1)N(Cc1cnn(C)c1)C(=O)c1cc(S(=O)(=O)NC3(C)CC3)ccc12] | (S)-2-ethynyl-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 157 | 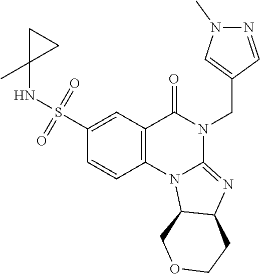 [SMILES: Cn1cc(CN2C(=O)c3cc(S(=O)(=O)NC4(C)CC4)ccc3N3C2=N[C@H]2CCOC[C@H]23)cn1] | (7aS,11aS)-6-((1-methyl-1H- pyrazol-4-yl)methyl)-N-(1- methylcyclopropyl)-5-oxo- 6,7a,8,9,11,11a-hexahydro-5H- pyrano[4′,3′:4,5]imidazo[1,2- a]quinazoline-3-sulfonamide |
| 158 | 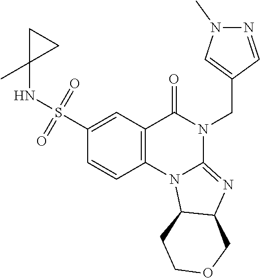 [SMILES: Cn1cc(CN2C(=O)c3cc(S(=O)(=O)NC4(C)CC4)ccc3N3C2=N[C@H]2COCC[C@H]23)cn1] | (7aR,11aR)-6-((1-methyl-1H- pyrazol-4-yl)methyl)-N-(1- methylcyclopropyl)-5-oxo- 5,6,7a,10,11,11a-hexahydro- 8H- pyrano[3′,4′:4,5]imidazo[1,2- a]quinazoline-3-sulfonamide |
| 159 | 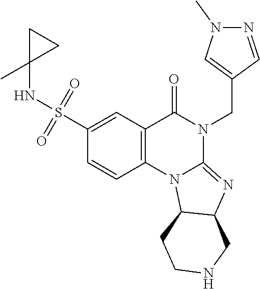 [SMILES: Cn1cc(CN2C(=O)c3cc(S(=O)(=O)NC4(C)CC4)ccc3N3C2=N[C@H]2CNCC[C@H]23)cn1] | (7aS,11aR)-6-((1-methyl-1H- pyrazol-4-yl)methyl)-N-(1- methylcyclopropyl)-5-oxo- 5,6,7a,8,9,10,11,11a- octahydropyrido[3′,4′:4,5]imidazo [1,2-a]quinazoline-3- sulfonamide |
| 160 | 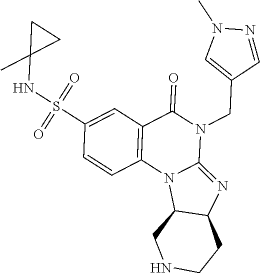 [SMILES: Cn1cc(CN2C(=O)c3cc(S(=O)(=O)NC4(C)CC4)ccc3N3C2=N[C@H]2CCNC[C@H]23)cn1] | (7aS,11aR)-6-((1-methyl-1H- pyrazol-4-yl)methyl)-N-(1- methylcyclopropyl)-5-oxo- 5,6,7a,8,9,10,11,11a- octahydropyrido[4′,3′:4,5]imidazo [1,2-a]quinazoline-3- sulfonamide |
| 161 | 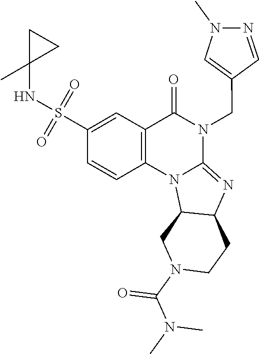 [SMILES: CN(C)C(=O)N1CC[C@@H]2N=C3N(Cc4cnn(C)c4)C(=O)c4cc(S(=O)(=O)NC5(C)CC5)ccc4N3[C@@H]2C1] | (7aS,11aR)-N,N-dimethyl-6- ((1-methyl-1H-pyrazol-4- yl)methyl)-3-(N-(1- methylcyclopropyl)sulfamoyl)- 5-oxo-6,7a,8,9,11,11a- hexahydropyrido[4′,3′:4,5]imidazo [1,2-a]quinazoline-10(5H)- carboxamide |
| 162 | 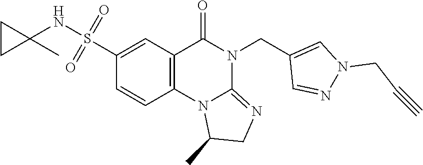 [SMILES: C#CCn1cc(CN2C(=O)c3cc(S(=O)(=O)NC4(C)CC4)ccc3N3C2=NC[C@H]3C)cn1] | (R)-1-methyl-N-(1- methylcyclopropyl)-5-oxo-4- ((1-(prop-2-yn-1-yl)-1H- pyrazol-4-yl)methyl)-1,2,4,5- tetrahydroimidazo[1,2- a]quinazoline-7-sulfonamide |
| 163 | 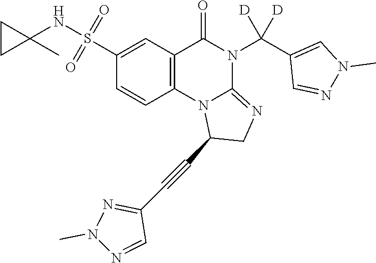 [SMILES: [2H]C([2H])(c1cnn(C)c1)N1C(=O)c2cc(S(=O)(=O)NC3(C)CC3)ccc2N2C1=NC[C@H]2C#Cc1cnn(C)n1] | (R)-4-((1-methyl-1H-pyrazol-4- yl)methyl-d2)-1-((2-methyl- 2H-1,2,3-triazol-4-yl)ethynyl)- N-(1-methylcyclopropyl)-5- oxo-1,2,4,5- tetrahydroimidazo[1,2- a]quinazoline-7-sulfonamide |
| 164 | 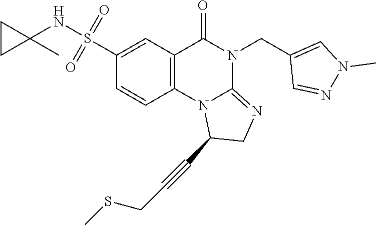 [SMILES: CSCC#C[C@@H]1CN=C2N(Cc3cnn(C)c3)C(=O)c3cc(S(=O)(=O)NC4(C)CC4)ccc3N21] | (R)-4-((1-methyl-1H-pyrazol-4- yl)methyl)-N-(1- methylcyclopropyl)-1-(3- (methylthio)prop-1-yn-1-yl)-5- oxo-1,2,4,5- tetrahydroimidazo[1,2- a]quinazoline-7-sulfonamide |
| 165 | 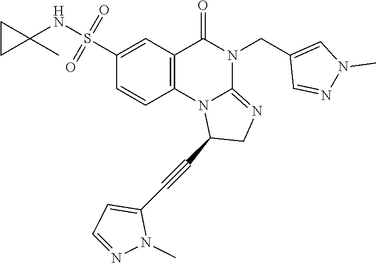 [SMILES: Cn1cc(CN2C(=O)c3cc(S(=O)(=O)NC4(C)CC4)ccc3N3C2=NC[C@H]3C#Cc2ccnn2C)cn1] | (R)-4-((1-methyl-1H-pyrazol-4- yl)methyl)-1-((1-methyl-1H- pyrazol-5-yl)ethynyl)-N-(1- methylcyclopropyl)-5-oxo- 1,2,4,5-tetrahydroimidazo[1,2- a]quinazoline-7-sulfonamide |
| 166 | 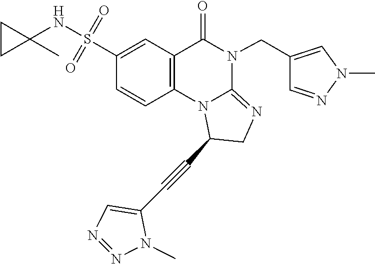 [SMILES: Cn1cc(CN2C(=O)c3cc(S(=O)(=O)NC4(C)CC4)ccc3N3C2=NC[C@H]3C#Cc2cnnn2C)cn1] | (R)-1-((1-methyl-1H-1,2,3- triazol-5-yl)ethynyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 167 | 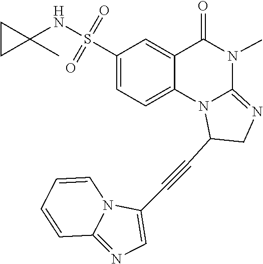 [SMILES: CN1C(=O)c2cc(S(=O)(=O)NC3(C)CC3)ccc2N2C1=NCC2C#Cc1cnc2ccccn12] | 1-(imidazo[1,2-a]pyridin-3- ylethynyl)-4-methyl-N-(1- methylcyclopropyl)-5-oxo- 1,2,4,5-tetrahydroimidazo[1,2- a]quinazoline-7-sulfonamide |
| 168 | 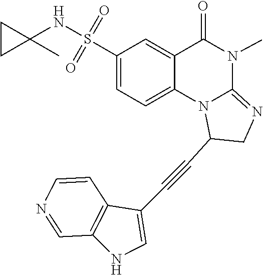 [SMILES: CN1C(=O)c2cc(S(=O)(=O)NC3(C)CC3)ccc2N2C1=NCC2C#Cc1c[nH]c2cnccc12] | 1-((1H-pyrrolo[2,3-c]pyridin-3- yl)ethynyl)-4-methyl-N-(1- methylcyclopropyl)-5-oxo- 1,2,4,5-tetrahydroimidazo[1,2- a]quinazoline-7-sulfonamide |
| 169 | 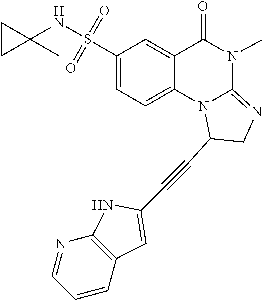 [SMILES: CN1C(=O)c2cc(S(=O)(=O)NC3(C)CC3)ccc2N2C1=NCC2C#Cc1cc2cccnc2[nH]1] | 1-((1H-pyrrolo[2,3-b]pyridin-2- yl)ethynyl)-4-methyl-N-(1- methylcyclopropyl)-5-oxo- 1,2,4,5-tetrahydroimidazo[1,2- a]quinazoline-7-sulfonamide |
| 170 | 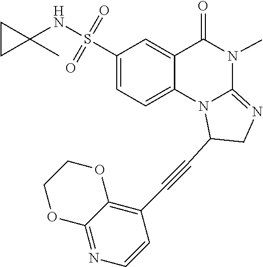 [SMILES: CN1C(=O)c2cc(S(=O)(=O)NC3(C)CC3)ccc2N2C1=NCC2C#Cc1ccnc2c1OCCO2] | 1-((2,3-dihydro- [1,4]dioxino[2,3-b]pyridin-8- yl)ethynyl)-4-methyl-N-(1- methylcyclopropyl)-5-oxo- 1,2,4,5-tetrahydroimidazo[1,2- a]quinazoline-7-sulfonamide |
| 171 | 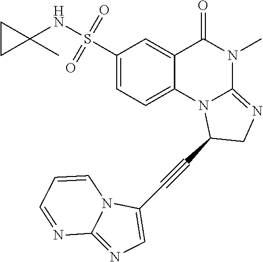 [SMILES: CN1C(=O)c2cc(S(=O)(=O)NC3(C)CC3)ccc2N2C1=NC[C@H]2C#Cc1cnc2ncccn12] | (R)-1-(imidazo[1,2- a]pyrimidin-3-ylethynyl)-4- methyl-N-(1- methylcyclopropyl)-5-oxo- 1,2,4,5-tetrahydroimidazo[1,2- a]quinazoline-7-sulfonamide |
| 172 | 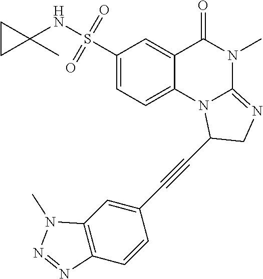 [SMILES: CN1C(=O)c2cc(S(=O)(=O)NC3(C)CC3)ccc2N2C1=NCC2C#Cc1ccc2nnn(C)c2c1] | 4-methyl-1-((1-methyl-1H- benzo[d][1,2,3]triazol-6- yl)ethynyl)-N-(1- methylcyclopropyl)-5-oxo- 1,2,4,5-tetrahydroimidazo[1,2- a]quinazoline-7-sulfonamide |
| 173 | 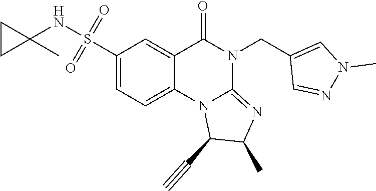 [SMILES: C#C[C@@H]1[C@H](C)N=C2N(Cc3cnn(C)c3)C(=O)c3cc(S(=O)(=O)NC4(C)CC4)ccc3N21] | (1R,2S)-1-ethynyl-2-methyl-4- ((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 174 | 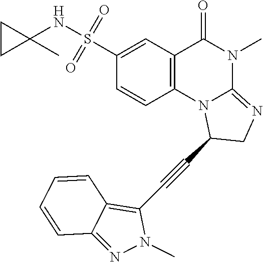 [SMILES: CN1C(=O)c2cc(S(=O)(=O)NC3(C)CC3)ccc2N2C1=NC[C@H]2C#Cc1c2ccccc2nn1C] | (R)-4-methyl-1-((2-methyl-2H- indazol-3-yl)ethynyl)-N-(1- methylcyclopropyl)-5-oxo- 1,2,4,5-tetrahydroimidazo[1,2- a]quinazoline-7-sulfonamide |
| 175 | 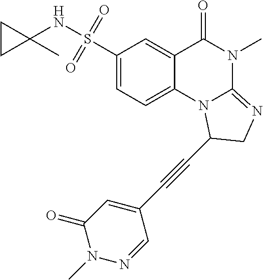 [SMILES: CN1C(=O)c2cc(S(=O)(=O)NC3(C)CC3)ccc2N2C1=NCC2C#Cc1cnn(C)c(=O)c1] | 4-methyl-1-((1-methyl-6-oxo- 1,6-dihydropyridazin-4- yl)ethynyl)-N-(1- methylcyclopropyl)-5-oxo- 1,2,4,5-tetrahydroimidazo[1,2- a]quinazoline-7-sulfonamide |
| 176 | 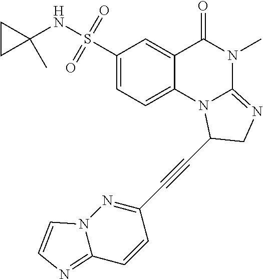 [SMILES: CN1C(=O)c2cc(S(=O)(=O)NC3(C)CC3)ccc2N2C1=NCC2C#Cc1ccc2nccn2n1] | 1-(imidazo[1,2-b]pyridazin-6- ylethynyl)-4-methyl-N-(1- methylcyclopropyl)-5-oxo- 1,2,4,5-tetrahydroimidazo[1,2- a]quinazoline-7-sulfonamide |
| 177 | 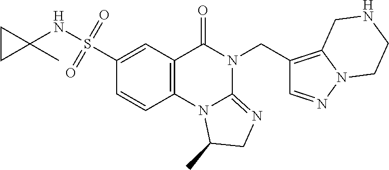 [SMILES: C[C@@H]1CN=C2N(Cc3cnn4c3CNCC4)C(=O)c3cc(S(=O)(=O)NC4(C)CC4)ccc3N21] | (R)-1-methyl-N-(1- methylcyclopropyl)-5-oxo-4- ((4,5,6,7- tetrahydropyrazolo[1,5- a]pyrazin-3-yl)methyl)-1,2,4,5- tetrahydroimidazo[1,2- a]quinazoline-7-sulfonamide |
| 178 | 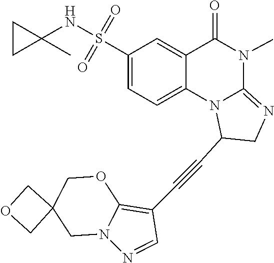 [SMILES: CN1C(=O)c2cc(S(=O)(=O)NC3(C)CC3)ccc2N2C1=NCC2C#Cc1cnn2c1OCC1(COC1)C2] | 1-((5′H, 7′H-spiro[oxetane-3,6′- pyrazolo[5,1-b][1,3]oxazin]-3′- yl)ethynyl)-4-methyl-N-(1- methylcyclopropyl)-5-oxo- 1,2,4,5-tetrahydroimidazo[1,2- a]quinazoline-7-sulfonamide |
| 179 | 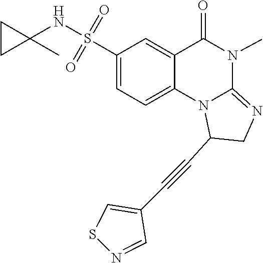 [SMILES: CN1C(=O)c2cc(S(=O)(=O)NC3(C)CC3)ccc2N2C1=NCC2C#Cc1cnsc1] | 1-(isothiazol-4-ylethynyl)-4- methyl-N-(1- methylcyclopropyl)-5-oxo- 1,2,4,5-tetrahydroimidazo[1,2- a]quinazoline-7-sulfonamide |
| 180 | 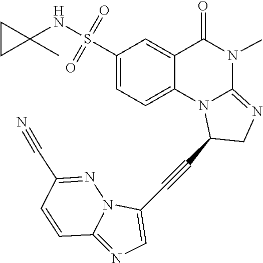 [SMILES: CN1C(=O)c2cc(S(=O)(=O)NC3(C)CC3)ccc2N2C1=NC[C@H]2C#Cc1cnc2ccc(C#N)nn12] | (R)-1-((6-cyanoimidazo[1,2- b]pyridazin-3-yl)ethynyl)-4- methyl-N-(1- methylcyclopropyl)-5-oxo- 1,2,4,5-tetrahydroimidazo[1,2- a]quinazoline-7-sulfonamide |
| 181 | 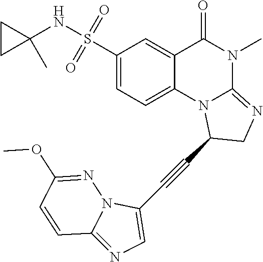 [SMILES: COc1ccc2ncc(C#C[C@@H]3CN=C4N(C)C(=O)c5cc(S(=O)(=O)NC6(C)CC6)ccc5N43)n2n1] | (R)-1-((6-methoxyimidazo[1,2- b]pyridazin-3-yl)ethynyl)-4- methyl-N-(1- methylcyclopropyl)-5-oxo- 1,2,4,5-tetrahydroimidazo[1,2- a]quinazoline-7-sulfonamide |
| 182 | 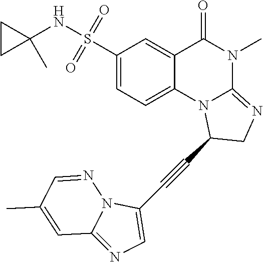 [SMILES: Cc1cnn2c(C#C[C@@H]3CN=C4N(C)C(=O)c5cc(S(=O)(=O)NC6(C)CC6)ccc5N43)cnc2c1] | (R)-4-methyl-N-(1- methylcyclopropyl)-1-((7- methylimidazo[1,2-b]pyridazin- 3-yl)ethynyl)-5-oxo-1,2,4,5- tetrahydroimidazo[1,2- a]quinazoline-7-sulfonamide |
| 183 | 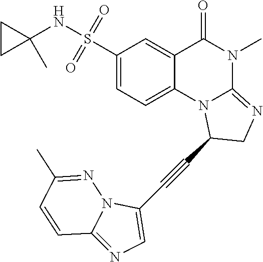 [SMILES: Cc1ccc2ncc(C#C[C@@H]3CN=C4N(C)C(=O)c5cc(S(=O)(=O)NC6(C)CC6)ccc5N43)n2n1] | (R)-4-methyl-N-(1- methylcyclopropyl)-1-((6- methylimidazo[1,2-b]pyridazin- 3-yl)ethynyl)-5-oxo-1,2,4,5- tetrahydroimidazo[1,2- a]quinazoline-7-sulfonamide |
| 184 | 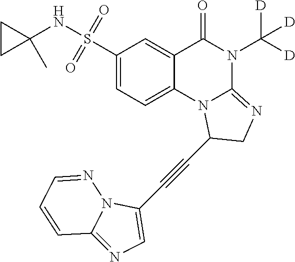 [SMILES: [2H]C([2H])([2H])N1C(=O)c2cc(S(=O)(=O)NC3(C)CC3)ccc2N2C1=NCC2C#Cc1cnc2cccnn12] | 1-(imidazo[1,2-b]pyridazin-3- ylethynyl)-4-(methyl-d3)-N-(1- methylcyclopropyl)-5-oxo- 1,2,4,5-tetrahydroimidazo[1,2- a]quinazoline-7-sulfonamide |
| 185 | 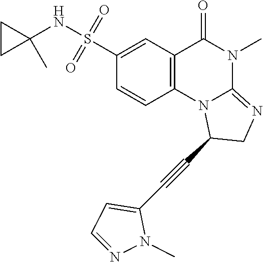 [SMILES: CN1C(=O)c2cc(S(=O)(=O)NC3(C)CC3)ccc2N2C1=NC[C@H]2C#Cc1ccnn1C] | (R)-4-methyl-1-((1-methyl-1H- pyrazol-5-yl)ethynyl)-N-(1- methylcyclopropyl)-5-oxo- 1,2,4,5-tetrahydroimidazo[1,2- a]quinazoline-7-sulfonamide |
| 186 | 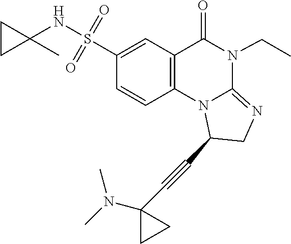 [SMILES: CCN1C(=O)c2cc(S(=O)(=O)NC3(C)CC3)ccc2N2C1=NC[C@H]2C#CC1(N(C)C)CC1] | (R)-1-((1- (dimethylamino)cyclopropyl) ethynyl)-4-ethyl-N-(1- methylcyclopropyl)-5-oxo- 1,2,4,5-tetrahydroimidazo[1,2- a]quinazoline-7-sulfonamide |
| 187 | 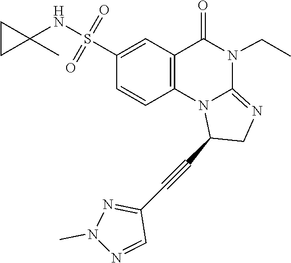 [SMILES: CCN1C(=O)c2cc(S(=O)(=O)NC3(C)CC3)ccc2N2C1=NC[C@H]2C#Cc1cnn(C)n1] | (R)-4-ethyl-1-((2-methyl-2H- 1,2,3-triazol-4-yl)ethynyl)-N- (1-methylcyclopropyl)-5-oxo- 1,2,4,5-tetrahydroimidazo[1,2- a]quinazoline-7-sulfonamide |
| 188 | 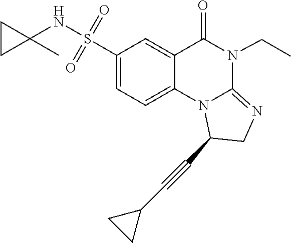 [SMILES: CCN1C(=O)c2cc(S(=O)(=O)NC3(C)CC3)ccc2N2C1=NC[C@H]2C#CC1CC1] | (R)-1-(cyclopropylethynyl)-4- ethyl-N-(1-methylcyclopropyl)- 5-oxo-1,2,4,5- tetrahydroimidazo[1,2- a]quinazoline-7-sulfonamide |
| 189 | 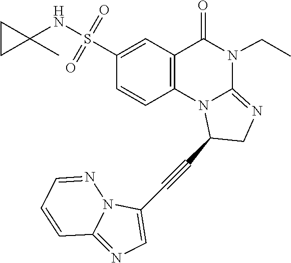 [SMILES: CCN1C(=O)c2cc(S(=O)(=O)NC3(C)CC3)ccc2N2C1=NC[C@H]2C#Cc1cnc2cccnn12] | (R)-4-ethyl-1-(imidazo[1,2- b]pyridazin-3-ylethynyl)-N-(1- methylcyclopropyl)-5-oxo- 1,2,4,5-tetrahydroimidazo[1,2- a]quinazoline-7-sulfonamide |
| 190 | 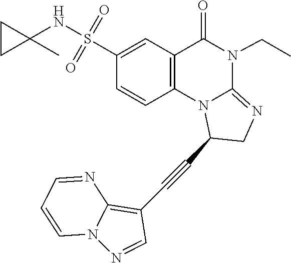 [SMILES: CCN1C(=O)c2cc(S(=O)(=O)NC3(C)CC3)ccc2N2C1=NC[C@H]2C#Cc1cnn2cccnc12] | (R)-4-ethyl-N-(1- methylcyclopropyl)-5-oxo-1- (pyrazolo[1,5-a]pyrimidin-3- ylethynyl)-1,2,4,5- tetrahydroimidazo[1,2- a]quinazoline-7-sulfonamide |
| 191 | 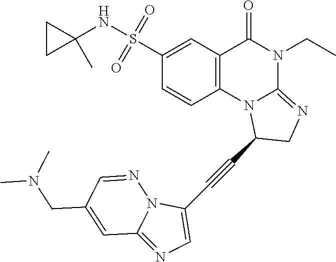 [SMILES: CCN1C(=O)c2cc(S(=O)(=O)NC3(C)CC3)ccc2N2C1=NC[C@H]2C#Cc1cnc2cc(CN(C)C)cnn12] | (R)-1-((7- ((dimethylamino)methyl)imidazo [1,2-b]pyridazin-3- yl)ethynyl)-4-ethyl-N-(1- methylcyclopropyl)-5-oxo- 1,2,4,5-tetrahydroimidazo[1,2- a]quinazoline-7-sulfonamide |
| 192 | 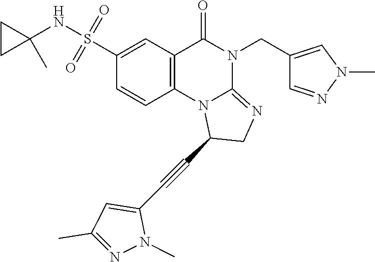 [SMILES: Cc1cc(C#C[C@@H]2CN=C3N(Cc4cnn(C)c4)C(=O)c4cc(S(=O)(=O)NC5(C)CC5)ccc4N32)n(C)n1] | (R)-1-((1,3-dimethyl-1H- pyrazol-5-yl)ethynyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 193 | 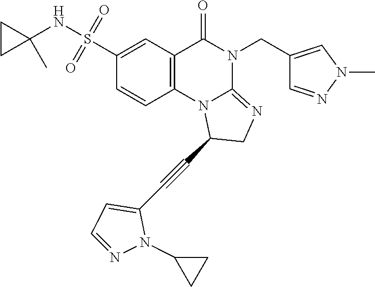 [SMILES: Cn1cc(CN2C(=O)c3cc(S(=O)(=O)NC4(C)CC4)ccc3N3C2=NC[C@H]3C#Cc2ccnn2C2CC2)cn1] | (R)-1-((1-cyclopropyl-1H- pyrazol-5-yl)ethynyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 194 | 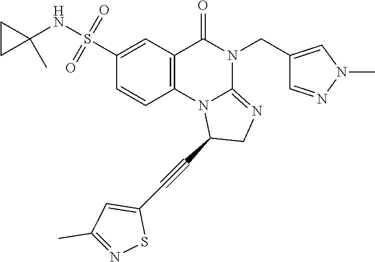 [SMILES: Cc1cc(C#C[C@@H]2CN=C3N(Cc4cnn(C)c4)C(=O)c4cc(S(=O)(=O)NC5(C)CC5)ccc4N32)sn1] | (R)-4-((1-methyl-1H-pyrazol-4- yl)methyl)-N-(1- methylcyclopropyl)-1-((3- methylisothiazol-5-yl)ethynyl)- 5-oxo-1,2,4,5- tetrahydroimidazo[1,2- a]quinazoline-7-sulfonamide |
| 195 | 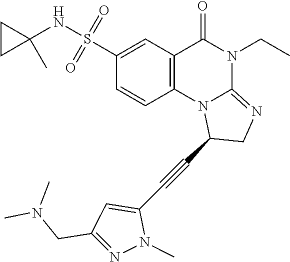 [SMILES: CCN1C(=O)c2cc(S(=O)(=O)NC3(C)CC3)ccc2N2C1=NC[C@H]2C#Cc1cc(CN(C)C)nn1C] | (R)-1-((3- ((dimethylamino)methyl)-1- methyl-1H-pyrazol-5- yl)ethynyl)-4-ethyl-N-(1- methylcyclopropyl)-5-oxo- 1,2,4,5-tetrahydroimidazo[1,2- a]quinazoline-7-sulfonamide |
| 196 | 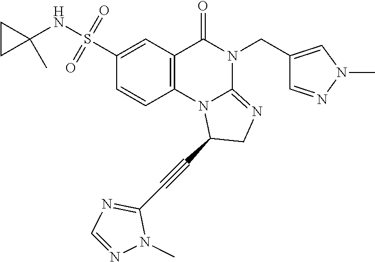 [SMILES: Cn1cc(CN2C(=O)c3cc(S(=O)(=O)NC4(C)CC4)ccc3N3C2=NC[C@H]3C#Cc2ncnn2C)cn1] | (R)-1-((1-methyl-1H-1,2,4- triazol-5-yl)ethynyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 197 | 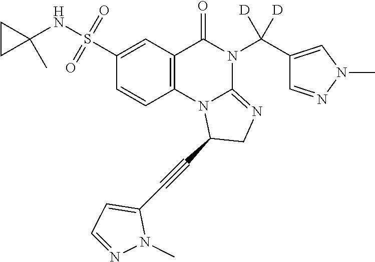 [SMILES: [2H]C([2H])(c1cnn(C)c1)N1C(=O)c2cc(S(=O)(=O)NC3(C)CC3)ccc2N2C1=NC[C@H]2C#Cc1ccnn1C] | (R)-4-((1-methyl-1H-pyrazol-4- yl)methyl-d2)-1-((1-methyl- 1H-pyrazol-5-yl)ethynyl)-N-(1- methylcyclopropyl)-5-oxo- 1,2,4,5-tetrahydroimidazo[1,2- a]quinazoline-7-sulfonamide |
| 198 | 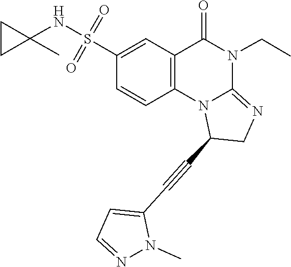 [SMILES: CCN1C(=O)c2cc(S(=O)(=O)NC3(C)CC3)ccc2N2C1=NC[C@H]2C#Cc1ccnn1C] | (R)-4-ethyl-1-((1-methyl-1H- pyrazol-5-yl)ethynyl)-N-(1- methylcyclopropyl)-5-oxo- 1,2,4,5-tetrahydroimidazo[1,2- a]quinazoline-7-sulfonamide |
| 199 | 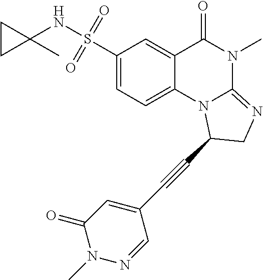 [SMILES: CN1C(=O)c2cc(S(=O)(=O)NC3(C)CC3)ccc2N2C1=NC[C@H]2C#Cc1cnn(C)c(=O)c1] | (R)-4-methyl-1-((1-methyl-6- oxo-1,6-dihydropyridazin-4- yl)ethynyl)-N-(1- methylcyclopropyl)-5-oxo- 1,2,4,5-tetrahydroimidazo[1,2- a]quinazoline-7-sulfonamide |
| 200 | 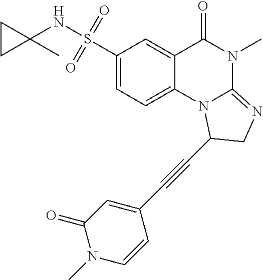 [SMILES: CN1C(=O)c2cc(S(=O)(=O)NC3(C)CC3)ccc2N2C1=NCC2C#Cc1ccn(C)c(=O)c1] | 4-methyl-1-((1-methyl-2-oxo- 1,2-dihydropyridin-4- yl)ethynyl)-N-(1- methylcyclopropyl)-5-oxo- 1,2,4,5-tetrahydroimidazo[1,2- a]quinazoline-7-sulfonamide |
| 201 | 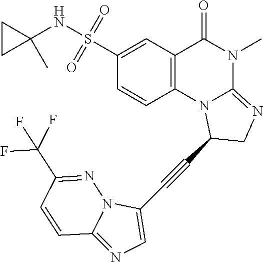 [SMILES: CN1C(=O)c2cc(S(=O)(=O)NC3(C)CC3)ccc2N2C1=NC[C@H]2C#Cc1cnc2ccc(C(F)(F)F)nn12] | (R)-4-methyl-N-(1- methylcyclopropyl)-5-oxo-1- ((6- (trifluoromethyl)imidazo[1,2- b]pyridazin-3-yl)ethynyl)- 1,2,4,5-tetrahydroimidazo[1,2- a]quinazoline-7-sulfonamide |
| 202 | 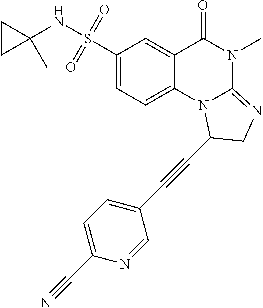 [SMILES: CN1C(=O)c2cc(S(=O)(=O)NC3(C)CC3)ccc2N2C1=NCC2C#Cc1ccc(C#N)nc1] | 1-((6-cyanopyridin-3- yl)ethynyl)-4-methyl-N-(1- methylcyclopropyl)-5-oxo- 1,2,4,5-tetrahydroimidazo[1,2- a]quinazoline-7-sulfonamide |
| 203 | 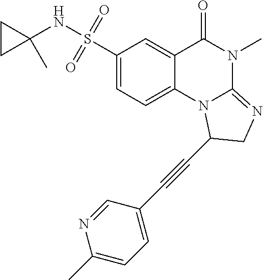 [SMILES: Cc1ccc(C#CC2CN=C3N(C)C(=O)c4cc(S(=O)(=O)NC5(C)CC5)ccc4N32)cn1] | 4-methyl-N-(1- methylcyclopropyl)-1-((6- methylpyridin-3-yl)ethynyl)-5- oxo-1,2,4,5- tetrahydroimidazo[1,2- a]quinazoline-7-sulfonamide |
| 204 | 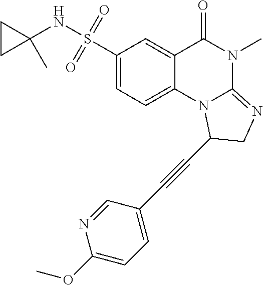 [SMILES: COc1ccc(C#CC2CN=C3N(C)C(=O)c4cc(S(=O)(=O)NC5(C)CC5)ccc4N32)cn1] | 1-((6-methoxypyridin-3- yl)ethynyl)-4-methyl-N-(1- methylcyclopropyl)-5-oxo- 1,2,4,5-tetrahydroimidazo[1,2- a]quinazoline-7-sulfonamide |
| 205 | 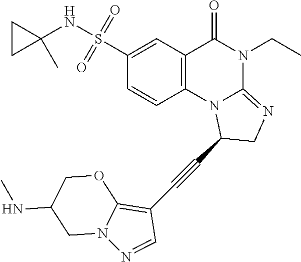 [SMILES: CCN1C(=O)c2cc(S(=O)(=O)NC3(C)CC3)ccc2N2C1=NC[C@H]2C#Cc1cnn2c1OCC(NC)C2] | (1R)-4-ethyl-1-((6- (methylamino)-6,7-dihydro-5H- pyrazolo[5,1-b][1,3]oxazin-3- yl)ethynyl)-N-(1- methylcyclopropyl)-5-oxo- 1,2,4,5-tetrahydroimidazo[1,2- a]quinazoline-7-sulfonamide |
| 206 | 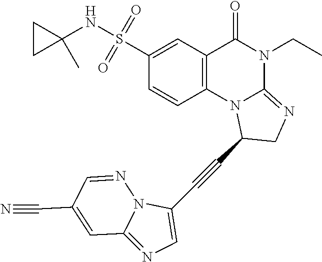 [SMILES: CCN1C(=O)c2cc(S(=O)(=O)NC3(C)CC3)ccc2N2C1=NC[C@H]2C#Cc1cnc2cc(C#N)cnn12] | (R)-1-((7-cyanoimidazo[1,2- b]pyridazin-3-yl)ethynyl)-4- ethyl-N-(1-methylcyclopropyl)- 5-oxo-1,2,4,5- tetrahydroimidazo[1,2- a]quinazoline-7-sulfonamide |
| 207 | 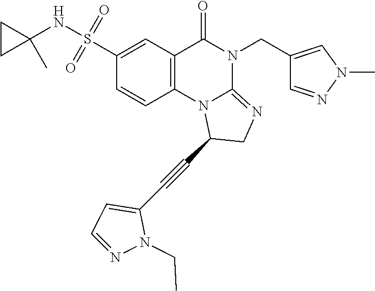 [SMILES: CCn1nccc1C#C[C@@H]1CN=C2N(Cc3cnn(C)c3)C(=O)c3cc(S(=O)(=O)NC4(C)CC4)ccc3N21] | (R)-1-((1-ethyl-1H-pyrazol-5- yl)ethynyl)-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 208 | 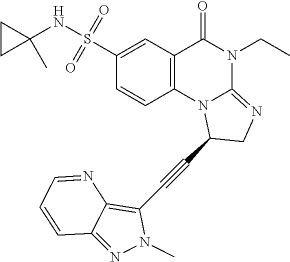 [SMILES: CCN1C(=O)c2cc(S(=O)(=O)NC3(C)CC3)ccc2N2C1=NC[C@H]2C#Cc1c2ncccc2nn1C] | (R)-4-ethyl-1-((2-methyl-2H- pyrazolo[4,3-b]pyridin-3- yl)ethynyl)-N-(1- methylcyclopropyl)-5-oxo- 1,2,4,5-tetrahydroimidazo[1,2- a]quinazoline-7-sulfonamide |
| 209 | 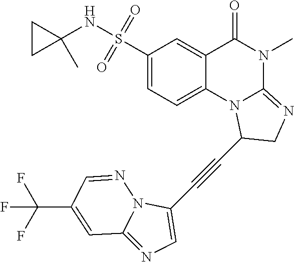 [SMILES: CN1C(=O)c2cc(S(=O)(=O)NC3(C)CC3)ccc2N2C1=NCC2C#Cc1cnc2cc(C(F)(F)F)cnn12] | 4-methyl-N-(1- methylcyclopropyl)-5-oxo-1- ((7- (trifluoromethyl)imidazo[1,2- b]pyridazin-3-yl)ethynyl)- 1,2,4,5-tetrahydroimidazo[1,2- a]quinazoline-7-sulfonamide |
| 210 | 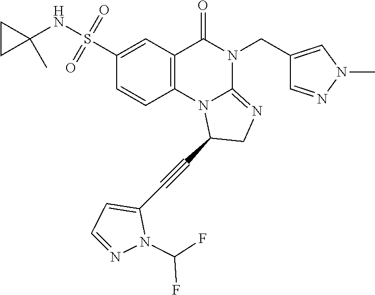 [SMILES: Cn1cc(CN2C(=O)c3cc(S(=O)(=O)NC4(C)CC4)ccc3N3C2=NC[C@H]3C#Cc2ccnn2C(F)F)cn1] | (R)-1-((1-(difluoromethyl)-1H- pyrazol-5-yl)ethynyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 211 | 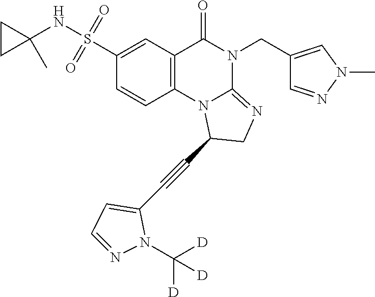 [SMILES: [2H]C([2H])([2H])n1nccc1C#C[C@@H]1CN=C2N(Cc3cnn(C)c3)C(=O)c3cc(S(=O)(=O)NC4(C)CC4)ccc3N21] | (R)-4-((1-methyl-1H-pyrazol-4- yl)methyl)-1-((1-(methyl-d3)- 1H-pyrazol-5-yl)ethynyl)-N-(1- methylcyclopropyl)-5-oxo- 1,2,4,5-tetrahydroimidazo[1,2- a]quinazoline-7-sulfonamide |
| 212 | 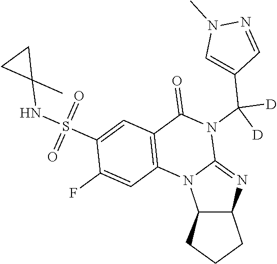 [SMILES: [2H]C([2H])(c1cnn(C)c1)N1C(=O)c2cc(S(=O)(=O)NC3(C)CC3)c(F)cc2N2C1=N[C@H]1CCC[C@H]12] | (7aS,10aR)-2-fluoro-6-((1- methyl-1H-pyrazol-4- yl)methyl-d2)-N-(1- methylcyclopropyl)-5-oxo- 6,7a,8,9,10,10a-hexahydro-5H- cyclopenta[4,5]imidazo[1,2- a]quinazoline-3-sulfonamide |
| 213 | 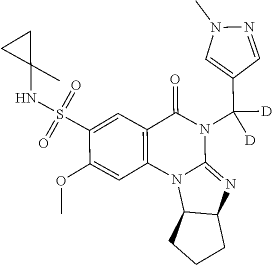 [SMILES: [2H]C([2H])(c1cnn(C)c1)N1C(=O)c2cc(S(=O)(=O)NC3(C)CC3)c(OC)cc2N2C1=N[C@H]1CCC[C@H]12] | (7aS,10aR)-2-methoxy-6-((1- methyl-1H-pyrazol-4- yl)methyl-d2)-N-(1- methylcyclopropyl)-5-oxo- 6,7a,8,9,10,10a-hexahydro-5H- cyclopenta[4,5]imidazo[1,2- a]quinazoline-3-sulfonamide |
| 214 | 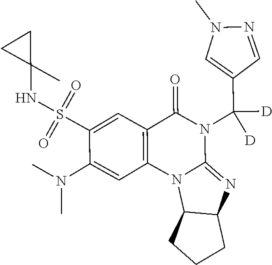 [SMILES: [2H]C([2H])(c1cnn(C)c1)N1C(=O)c2cc(S(=O)(=O)NC3(C)CC3)c(N(C)C)cc2N2C1=N[C@H]1CCC[C@H]12] | (7aS,10aR)-2-(dimethylamino)- 6-((1-methyl-1H-pyrazol-4- yl)methyl-d2)-N-(1- methylcyclopropyl)-5-oxo- 6,7a,8,9,10,10a-hexahydro-5H- cyclopenta[4,5]imidazo[1,2- a]quinazoline-3-sulfonamide |
| 215 | 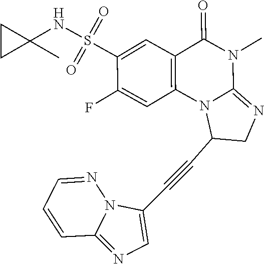 [SMILES: CN1C(=O)c2cc(S(=O)(=O)NC3(C)CC3)c(F)cc2N2C1=NCC2C#Cc1cnc2cccnn12] | 8-fluoro-1-(imidazo[1,2- b]pyridazin-3-ylethynyl)-4- methyl-N-(1- methylcyclopropyl)-5-oxo- 1,2,4,5-tetrahydroimidazo[1,2- a]quinazoline-7-sulfonamide |
| 216 | 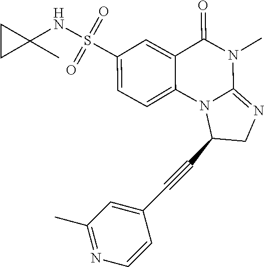 [SMILES: Cc1cc(C#C[C@@H]2CN=C3N(C)C(=O)c4cc(S(=O)(=O)NC5(C)CC5)ccc4N32)ccn1] | (R)-4-methyl-N-(1- methylcyclopropyl)-1-((2- methylpyridin-4-yl)ethynyl)-5- oxo-1,2,4,5- tetrahydroimidazo[1,2- a]quinazoline-7-sulfonamide |
| 217 | 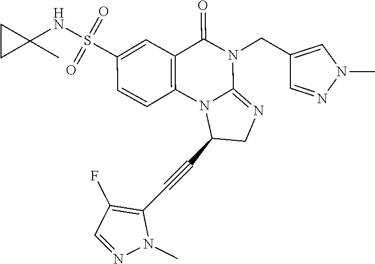 [SMILES: Cn1cc(CN2C(=O)c3cc(S(=O)(=O)NC4(C)CC4)ccc3N3C2=NC[C@H]3C#Cc2c(F)cnn2C)cn1] | (R)-1-((4-fluoro-1-methyl-1H- pyrazol-5-yl)ethynyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 218 | 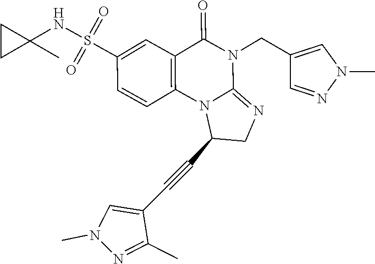 [SMILES: Cc1nn(C)cc1C#C[C@@H]1CN=C2N(Cc3cnn(C)c3)C(=O)c3cc(S(=O)(=O)NC4(C)CC4)ccc3N21] | (R)-1-((1,3-dimethyl-1H- pyrazol-4-yl)ethynyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 219 | 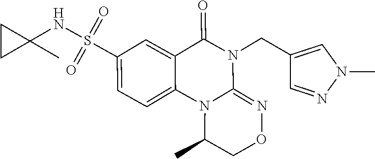 [SMILES: C[C@@H]1CON=C2N(Cc3cnn(C)c3)C(=O)c3cc(S(=O)(=O)NC4(C)CC4)ccc3N21] | (R)-1-methyl-5-((1-methyl-1H- pyrazol-4-yl)methyl)-N-(1- methylcyclopropyl)-6-oxo- 1,2,5,6-tetrahydro- [1,2,4]oxadiazino[4,3- a]quinazoline-8-sulfonamide |
| 220 | 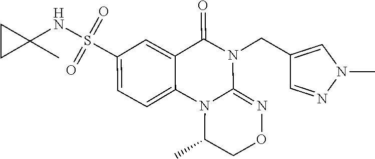 [SMILES: C[C@H]1CON=C2N(Cc3cnn(C)c3)C(=O)c3cc(S(=O)(=O)NC4(C)CC4)ccc3N21] | (S)-1-methyl-5-((1-methyl-1H- pyrazol-4-yl)methyl)-N-(1- methylcyclopropyl)-6-oxo- 1,2,5,6-tetrahydro- [1,2,4]oxadiazino[4,3- a]quinazoline-8-sulfonamide |
| 221 | 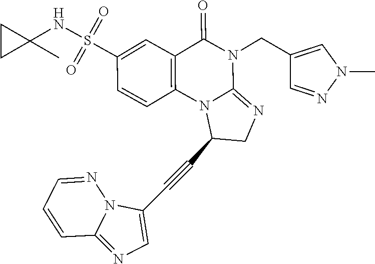 [SMILES: Cn1cc(CN2C(=O)c3cc(S(=O)(=O)NC4(C)CC4)ccc3N3C2=NC[C@H]3C#Cc2cnc3cccnn23)cn1] | (R)-1-(imidazo[1,2- b]pyridazin-3-ylethynyl)-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide |
| 222 | 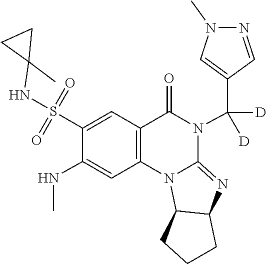 [SMILES: [2H]C([2H])(c1cnn(C)c1)N1C(=O)c2cc(S(=O)(=O)NC3(C)CC3)c(NC)cc2N2C1=N[C@H]1CCC[C@H]12] | (7aS,10aR)-6-((1-methyl-1H- pyrazol-4-yl)methyl-d2)-2- (methylamino)-N-(1- methylcyclopropyl)-5-oxo- 6,7a,8,9,10,10a-hexahydro-5H- cyclopenta[4,5]imidazo[1,2- a]quinazoline-3-sulfonamide |
| 223 | 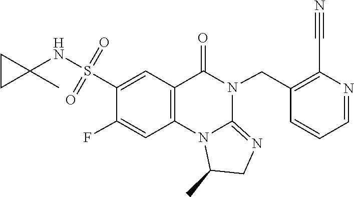 [SMILES: C[C@@H]1CN=C2N(Cc3cccnc3C#N)C(=O)c3cc(S(=O)(=O)NC4(C)CC4)c(F)cc3N21] | (R)-4-((2-cyanopyridin-3- yl)methyl)-8-fluoro-1-methyl- N-(1-methylcyclopropyl)-5- oxo-1,2,4,5- tetrahydroimidazo[1,2- a]quinazoline-7-sulfonamide |
| 224 | 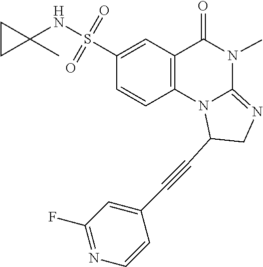 [SMILES: CN1C(=O)c2cc(S(=O)(=O)NC3(C)CC3)ccc2N2C1=NCC2C#Cc1ccnc(F)c1] | 1-((2-fluoropyridin-4- yl)ethynyl)-4-methyl-N-(1- methylcyclopropyl)-5-oxo- 1,2,4,5-tetrahydroimidazo[1,2- a]quinazoline-7-sulfonamide |
| 225 | 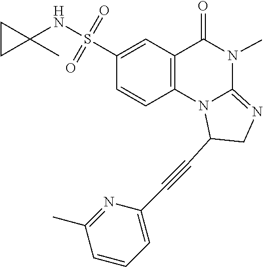 [SMILES: Cc1cccc(C#CC2CN=C3N(C)C(=O)c4cc(S(=O)(=O)NC5(C)CC5)ccc4N32)n1] | 4-methyl-N-(1- methylcyclopropyl)-1-((6- methylpyridin-2-yl)ethynyl)-5- oxo-1,2,4,5- tetrahydroimidazo[1,2- a]quinazoline-7-sulfonamide |
| 226 | 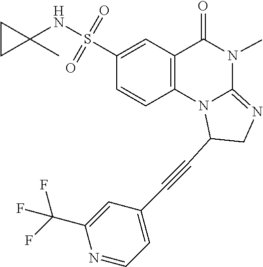 [SMILES: CN1C(=O)c2cc(S(=O)(=O)NC3(C)CC3)ccc2N2C1=NCC2C#Cc1ccnc(C(F)(F)F)c1] | 4-methyl-N-(1- methylcyclopropyl)-5-oxo-1- ((2-(trifluoromethyl)pyridin-4- yl)ethynyl)-1,2,4,5- tetrahydroimidazo[1,2- a]quinazoline-7-sulfonamide |
| 227 | 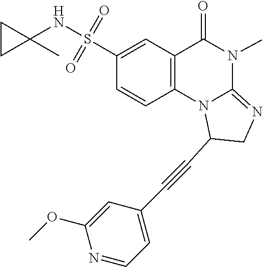 [SMILES: COc1cc(C#CC2CN=C3N(C)C(=O)c4cc(S(=O)(=O)NC5(C)CC5)ccc4N32)ccn1] | 1-((2-methoxypyridin-4- yl)ethynyl)-4-methyl-N-(1- methylcyclopropyl)-5-oxo- 1,2,4,5-tetrahydroimidazo[1,2- a]quinazoline-7-sulfonamide |
| 228 | 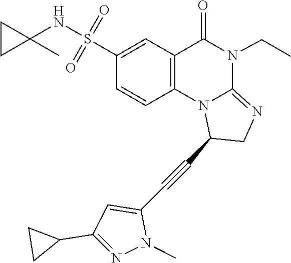 [SMILES: CCN1C(=O)c2cc(S(=O)(=O)NC3(C)CC3)ccc2N2C1=NC[C@H]2C#Cc1cc(C2CC2)nn1C] | (R)-1-((3-cyclopropyl-1- methyl-1H-pyrazol-5- yl)ethynyl)-4-ethyl-N-(1- methylcyclopropyl)-5-oxo- 1,2,4,5-tetrahydroimidazo[1,2- a]quinazoline-7-sulfonamide |
| 229 | 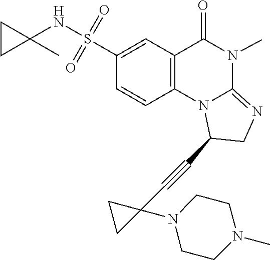 [SMILES: CN1CCN(C2(C#C[C@@H]3CN=C4N(C)C(=O)c5cc(S(=O)(=O)NC6(C)CC6)ccc5N43)CC2)CC1] | (R)-4-methyl-N-(1- methylcyclopropyl)-1-((1-(4- methylpiperazin-1- yl)cyclopropyl)ethynyl)-5-oxo- 1,2,4,5-tetrahydroimidazo[1,2- a]quinazoline-7-sulfonamide |
| 230 | 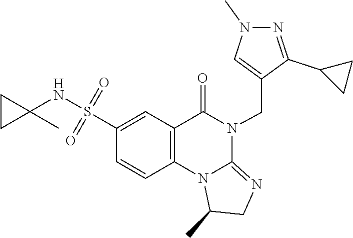 [SMILES: C[C@@H]1CN=C2N(Cc3cn(C)nc3C3CC3)C(=O)c3cc(S(=O)(=O)NC4(C)CC4)ccc3N21] | (R)-4-((3-cyclopropyl-1- methyl-1H-pyrazol-4- yl)methyl)-1-methyl-N-(1- methylcyclopropyl)-5-oxo- 1,2,4,5-tetrahydroimidazo[1,2- a]quinazoline-7-sulfonamide |
| 231 | 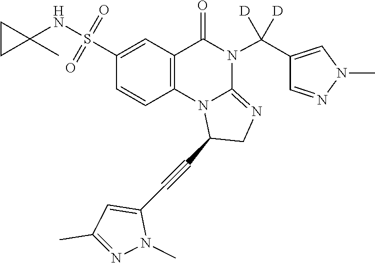 [SMILES: [2H]C([2H])(c1cnn(C)c1)N1C(=O)c2cc(S(=O)(=O)NC3(C)CC3)ccc2N2C1=NC[C@H]2C#Cc1cc(C)nn1C] | (R)-1-((1,3-dimethyl-1H- pyrazol-5-yl)ethynyl)-4-((1- methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 232 | 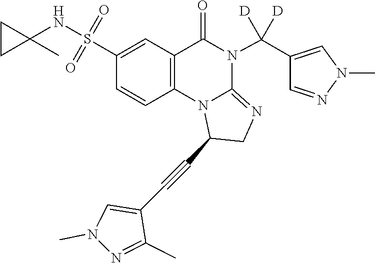 [SMILES: [2H]C([2H])(c1cnn(C)c1)N1C(=O)c2cc(S(=O)(=O)NC3(C)CC3)ccc2N2C1=NC[C@H]2C#Cc1cn(C)nc1C] | (R)-1-((1,3-dimethyl-1H- pyrazol-4-yl)ethynyl)-4-((1- methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 233 | 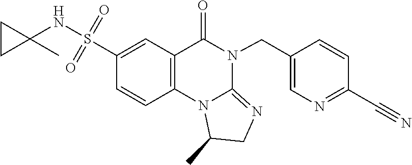 [SMILES: C[C@@H]1CN=C2N(Cc3ccc(C#N)nc3)C(=O)c3cc(S(=O)(=O)NC4(C)CC4)ccc3N21] | (R)-4-((6-cyanopyridin-3- yl)methyl)-1-methyl-N-(1- methylcyclopropyl)-5-oxo- 1,2,4,5-tetrahydroimidazo[1,2- a]quinazoline-7-sulfonamide |
| 234 | 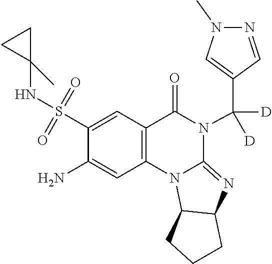 [SMILES: [2H]C([2H])(c1cnn(C)c1)N1C(=O)c2cc(S(=O)(=O)NC3(C)CC3)c(N)cc2N2C1=N[C@H]1CCC[C@H]12] | (7aS,10aR)-2-amino-6-((1- methyl-1H-pyrazol-4- yl)methyl-d2)-N-(1- methylcyclopropyl)-5-oxo- 6,7a,8,9,10,10a-hexahydro-5H- cyclopenta[4,5]imidazo[1,2- a]quinazoline-3-sulfonamide |
| 235 | 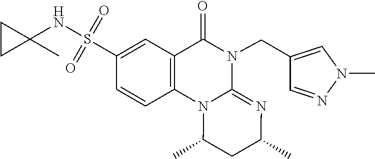 [SMILES: C[C@@H]1C[C@H](C)N2C(=N1)N(Cc1cnn(C)c1)C(=O)c1cc(S(=O)(=O)NC3(C)CC3)ccc12] | (1S,3R)-1,3-dimethyl-5-((1- methyl-1H-pyrazol-4- yl)methyl)-N-(1- methylcyclopropyl)-6-oxo- 2,3,5,6-tetrahydro-1H- pyrimido[1,2-a]quinazoline-8- sulfonamide |
| 236 | 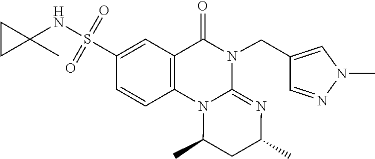 [SMILES: C[C@@H]1C[C@@H](C)N2C(=N1)N(Cc1cnn(C)c1)C(=O)c1cc(S(=O)(=O)NC3(C)CC3)ccc12] | (1R,3R)-1,3-dimethyl-5-((1- methyl-1H-pyrazol-4- yl)methyl)-N-(1- methylcyclopropyl)-6-oxo- 2,3,5,6-tetrahydro-1H- pyrimido[1,2-a]quinazoline-8- sulfonamide |
| 237 | 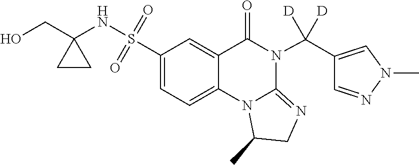 [SMILES: [2H]C([2H])(c1cnn(C)c1)N1C(=O)c2cc(S(=O)(=O)NC3(CO)CC3)ccc2N2C1=NC[C@H]2C] | (R)-N-(1- (hydroxymethyl)cyclopropyl)- 1-methyl-4-((1-methyl-1H- pyrazol-4-yl)methyl-d2)-5-oxo- 1,2,4,5-tetrahydroimidazo[1,2- a]quinazoline-7-sulfonamide |
| 238 | 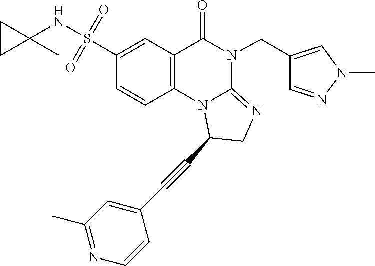 [SMILES: Cc1cc(C#C[C@@H]2CN=C3N(Cc4cnn(C)c4)C(=O)c4cc(S(=O)(=O)NC5(C)CC5)ccc4N32)ccn1] | (R)-4-((1-methyl-1H-pyrazol-4- yl)methyl)-N-(1- methylcyclopropyl)-1-((2- methylpyridin-4-yl)ethynyl)-5- oxo-1,2,4,5- tetrahydroimidazo[1,2- a]quinazoline-7-sulfonamide |
| 239 | 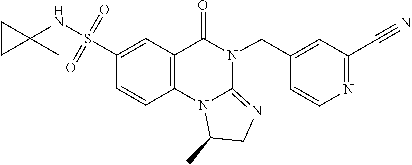 [SMILES: C[C@@H]1CN=C2N(Cc3ccnc(C#N)c3)C(=O)c3cc(S(=O)(=O)NC4(C)CC4)ccc3N21] | (R)-4-((2-cyanopyridin-4- yl)methyl)-1-methyl-N-(1- methylcyclopropyl)-5-oxo- 1,2,4,5-tetrahydroimidazo[1,2- a]quinazoline-7-sulfonamide |
| 240 | 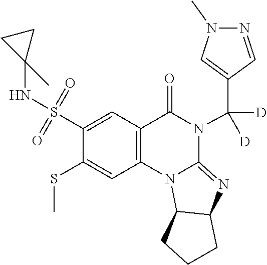 [SMILES: [2H]C([2H])(c1cnn(C)c1)N1C(=O)c2cc(S(=O)(=O)NC3(C)CC3)c(SC)cc2N2C1=N[C@H]1CCC[C@H]12] | (7aS,10aR)-6-((1-methyl-1H- pyrazol-4-yl)methyl-d2)-N-(1- methylcyclopropyl)-2- (methylthio)-5-oxo- 6,7a,8,9,10,10a-hexahydro-5H- cyclopenta[4,5]imidazo[1,2- a]quinazoline-3-sulfonamide |
| 241 | 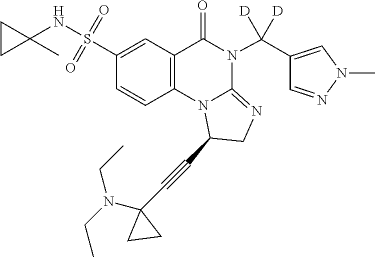 [SMILES: [2H]C([2H])(c1cnn(C)c1)N1C(=O)c2cc(S(=O)(=O)NC3(C)CC3)ccc2N2C1=NC[C@H]2C#CC1(N(CC)CC)CC1] | (R)-1-((1- (diethylamino)cyclopropyl)ethynyl)- 4-((1-methyl-1H-pyrazol- 4-yl)methyl-d2)-N-(1- methylcyclopropyl)-5-oxo- 1,2,4,5-tetrahydroimidazo[1,2- a]quinazoline-7-sulfonamide |
| 242 | 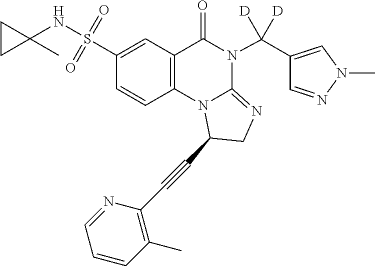 [SMILES: [2H]C([2H])(c1cnn(C)c1)N1C(=O)c2cc(S(=O)(=O)NC3(C)CC3)ccc2N2C1=NC[C@H]2C#Cc1ncccc1C] | (R)-4-((1-methyl-1H-pyrazol-4- yl)methyl-d2)-N-(1- methylcyclopropyl)-1-((3- methylpyridin-2-yl)ethynyl)-5- oxo-1,2,4,5- tetrahydroimidazo[1,2- a]quinazoline-7-sulfonamide |
| 243 | 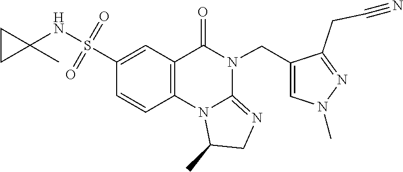 [SMILES: C[C@@H]1CN=C2N(Cc3cn(C)nc3CC#N)C(=O)c3cc(S(=O)(=O)NC4(C)CC4)ccc3N21] | (R)-4-((3-(cyanomethyl)-1- methyl-1H-pyrazol-4- yl)methyl)-1-methyl-N-(1- methylcyclopropyl)-5-oxo- 1,2,4,5-tetrahydroimidazo[1,2- a]quinazoline-7-sulfonamide |
| 244 | 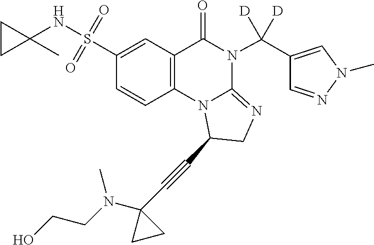 [SMILES: [2H]C([2H])(c1cnn(C)c1)N1C(=O)c2cc(S(=O)(=O)NC3(C)CC3)ccc2N2C1=NC[C@H]2C#CC1(N(C)CCO)CC1] | (R)-1-((1-((2- hydroxyethyl)(methyl)amino) cyclopropyl)ethynyl)-4-((1- methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 245 | 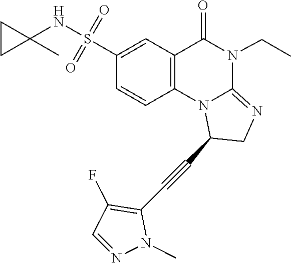 [SMILES: CCN1C(=O)c2cc(S(=O)(=O)NC3(C)CC3)ccc2N2C1=NC[C@H]2C#Cc1c(F)cnn1C] | (R)-4-ethyl-1-((4-fluoro-1- methyl-1H-pyrazol-5- yl)ethynyl)-N-(1- methylcyclopropyl)-5-oxo- 1,2,4,5-tetrahydroimidazo[1,2- a]quinazoline-7-sulfonamide |
| 246 | 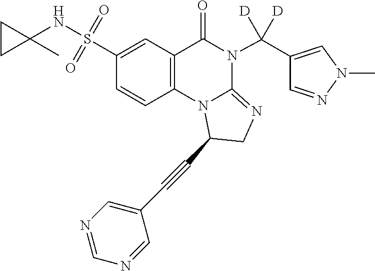 [SMILES: [2H]C([2H])(c1cnn(C)c1)N1C(=O)c2cc(S(=O)(=O)NC3(C)CC3)ccc2N2C1=NC[C@H]2C#Cc1cncnc1] | (R)-4-((1-methyl-1H-pyrazol-4- yl)methyl-d2)-N-(1- methylcyclopropyl)-5-oxo-1- (pyrimidin-5-ylethynyl)- 1,2,4,5-tetrahydroimidazo[1,2- a]quinazoline-7-sulfonamide |
| 247 | 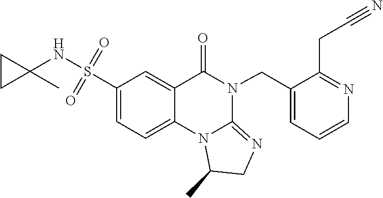 [SMILES: C[C@@H]1CN=C2N(Cc3cccnc3CC#N)C(=O)c3cc(S(=O)(=O)NC4(C)CC4)ccc3N21] | (R)-4-((2- (cyanomethyl)pyridin-3- yl)methyl)-1-methyl-N-(1- methylcyclopropyl)-5-oxo- 1,2,4,5-tetrahydroimidazo[1,2- a]quinazoline-7-sulfonamide |
| 248 | 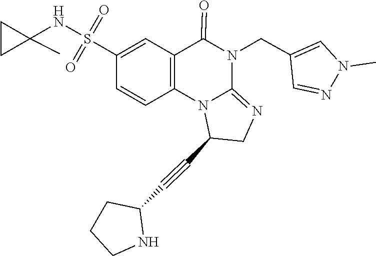 [SMILES: Cn1cc(CN2C(=O)c3cc(S(=O)(=O)NC4(C)CC4)ccc3N3C2=NC[C@H]3C#C[C@H]2CCCN2)cn1] | (R)-4-((1-methyl-1H-pyrazol-4- yl)methyl)-N-(1- methylcyclopropyl)-5-oxo-1- (((R)-pyrrolidin-2-yl)ethynyl)- 1,2,4,5-tetrahydroimidazo[1,2- a]quinazoline-7-sulfonamide |
| 249 | 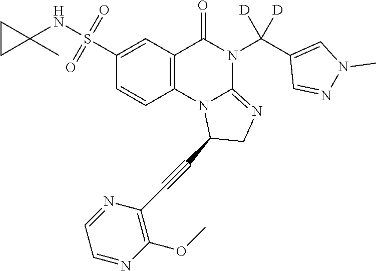 [SMILES: [2H]C([2H])(c1cnn(C)c1)N1C(=O)c2cc(S(=O)(=O)NC3(C)CC3)ccc2N2C1=NC[C@H]2C#Cc1nccnc1OC] | (R)-1-((3-methoxypyrazin-2- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 250 | 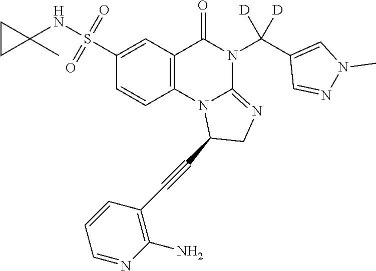 [SMILES: [2H]C([2H])(c1cnn(C)c1)N1C(=O)c2cc(S(=O)(=O)NC3(C)CC3)ccc2N2C1=NC[C@H]2C#Cc1cccnc1N] | (R)-1-((2-aminopyridin-3- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 251 | 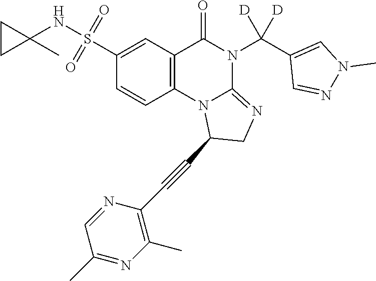 [SMILES: [2H]C([2H])(c1cnn(C)c1)N1C(=O)c2cc(S(=O)(=O)NC3(C)CC3)ccc2N2C1=NC[C@H]2C#Cc1ncc(C)nc1C] | (R)-1-((3,5-dimethylpyrazin-2- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 252 | 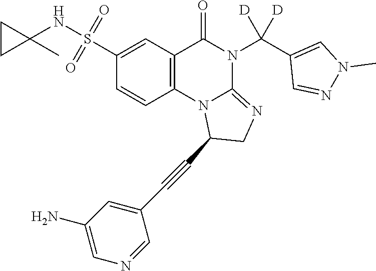 [SMILES: [2H]C([2H])(c1cnn(C)c1)N1C(=O)c2cc(S(=O)(=O)NC3(C)CC3)ccc2N2C1=NC[C@H]2C#Cc1cncc(N)c1] | (R)-1-((5-aminopyridin-3- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 253 | 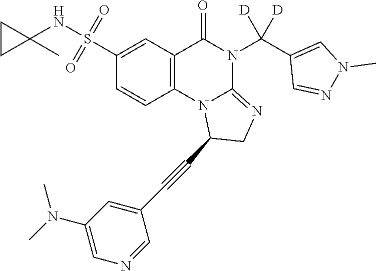 [SMILES: [2H]C([2H])(c1cnn(C)c1)N1C(=O)c2cc(S(=O)(=O)NC3(C)CC3)ccc2N2C1=NC[C@H]2C#Cc1cncc(N(C)C)c1] | (R)-1-((5- (dimethylamino)pyridin-3- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 254 | 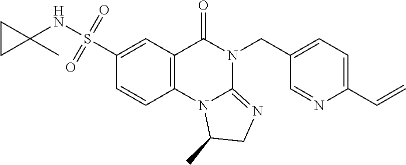 [SMILES: C=Cc1ccc(CN2C(=O)c3cc(S(=O)(=O)NC4(C)CC4)ccc3N3C2=NC[C@H]3C)cn1] | (R)-1-methyl-N-(1- methylcyclopropyl)-5-oxo-4- ((6-vinylpyridin-3-yl)methyl)- 1,2,4,5-tetrahydroimidazo[1,2- a]quinazoline-7-sulfonamide |
| 255 | 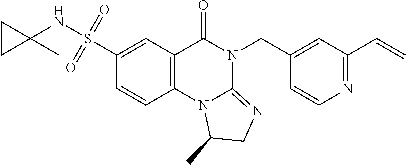 [SMILES: C=Cc1cc(CN2C(=O)c3cc(S(=O)(=O)NC4(C)CC4)ccc3N3C2=NC[C@H]3C)ccn1] | (R)-1-methyl-N-(1- methylcyclopropyl)-5-oxo-4- ((2-vinylpyridin-4-yl)methyl)- 1,2,4,5-tetrahydroimidazo[1,2- a]quinazoline-7-sulfonamide |
| 256 | 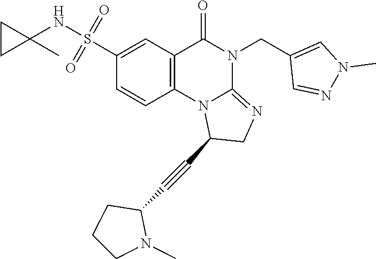 [SMILES: CN1CCC[C@@H]1C#C[C@@H]1CN=C2N(Cc3cnn(C)c3)C(=O)c3cc(S(=O)(=O)NC4(C)CC4)ccc3N21] | (R)-4-((1-methyl-1H-pyrazol-4- yl)methyl)-N-(1- methylcyclopropyl)-1-(((R)-1- methylpyrrolidin-2-yl)ethynyl)- 5-oxo-1,2,4,5- tetrahydroimidazo[1,2- a]quinazoline-7-sulfonamide |
| 257 | 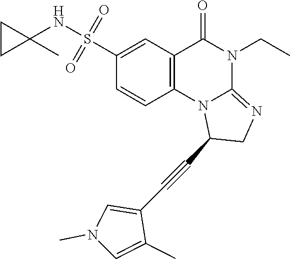 [SMILES: CCN1C(=O)c2cc(S(=O)(=O)NC3(C)CC3)ccc2N2C1=NC[C@H]2C#Cc1cn(C)cc1C] | (R)-1-((2,5-dimethyl-2H-1,2,3- triazol-4-yl)ethynyl)-4-ethyl-N- (1-methylcyclopropyl)-5-oxo- 1,2,4,5-tetrahydroimidazo[1,2- a]quinazoline-7-sulfonamide |
| 258 | 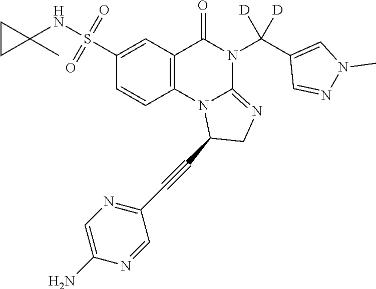 [SMILES: [2H]C([2H])(c1cnn(C)c1)N1C(=O)c2cc(S(=O)(=O)NC3(C)CC3)ccc2N2C1=NC[C@H]2C#Cc1cnc(N)cn1] | (R)-1-((2-aminopyrimidin-5- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 259 | 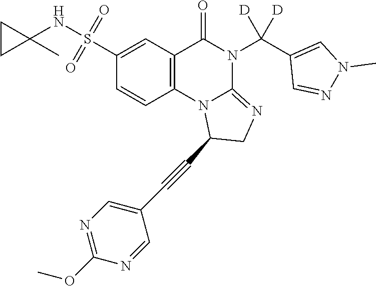 [SMILES: [2H]C([2H])(c1cnn(C)c1)N1C(=O)c2cc(S(=O)(=O)NC3(C)CC3)ccc2N2C1=NC[C@H]2C#Cc1cnc(OC)nc1] | (R)-1-((2-methoxypyrimidin-5- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 260 | 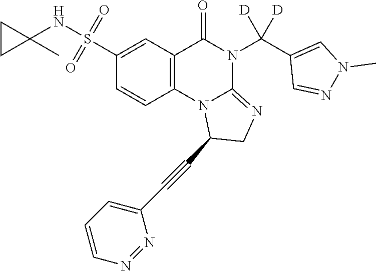 [SMILES: [2H]C([2H])(c1cnn(C)c1)N1C(=O)c2cc(S(=O)(=O)NC3(C)CC3)ccc2N2C1=NC[C@H]2C#Cc1cccnn1] | (R)-4-((1-methyl-1H-pyrazol-4- yl)methyl-d2)-N-(1- methylcyclopropyl)-5-oxo-1- (pyridazin-3-ylethynyl)-1,2,4,5- tetrahydroimidazo[1,2- a]quinazoline-7-sulfonamide |
| 261 | 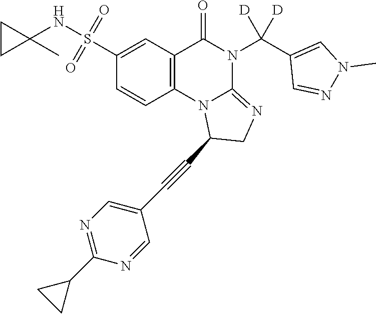 [SMILES: [2H]C([2H])(c1cnn(C)c1)N1C(=O)c2cc(S(=O)(=O)NC3(C)CC3)ccc2N2C1=NC[C@H]2C#Cc1cnc(C2CC2)nc1] | (R)-1-((2- cyclopropylpyrimidin-5- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 262 | 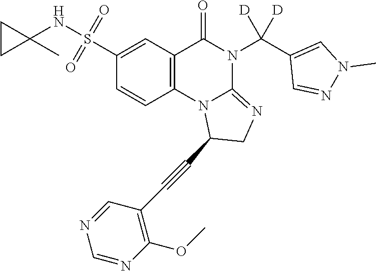 [SMILES: [2H]C([2H])(c1cnn(C)c1)N1C(=O)c2cc(S(=O)(=O)NC3(C)CC3)ccc2N2C1=NC[C@H]2C#Cc1cncnc1OC] | (R)-1-((4-methoxypyrimidin-5- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 263 | 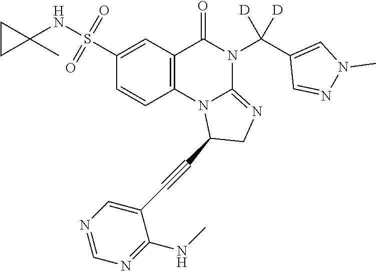 [SMILES: [2H]C([2H])(c1cnn(C)c1)N1C(=O)c2cc(S(=O)(=O)NC3(C)CC3)ccc2N2C1=NC[C@H]2C#Cc1cncnc1NC] | (R)-4-((1-methyl-1H-pyrazol-4- yl)methyl-d2)-1-((4- (methylamino)pyrimidin-5- yl)ethynyl)-N-(1- methylcyclopropyl)-5-oxo- 1,2,4,5-tetrahydroimidazo[1,2- a]quinazoline-7-sulfonamide |
| 264 | 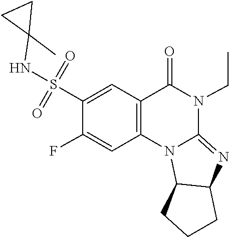 [SMILES: CCN1C(=O)c2cc(S(=O)(=O)NC3(C)CC3)c(F)cc2N2C1=N[C@H]1CCC[C@H]12] | (7aS,10aR)-6-ethyl-2-fluoro-N- (1-methylcyclopropyl)-5-oxo- 6,7a,8,9,10,10a-hexahydro-5H- cyclopenta[4,5]imidazo[1,2- a]quinazoline-3-sulfonamide |
| 265 | 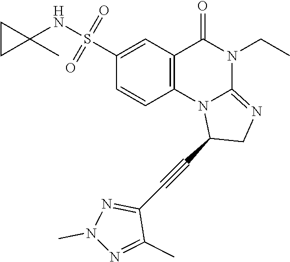 [SMILES: CCN1C(=O)c2cc(S(=O)(=O)NC3(C)CC3)ccc2N2C1=NC[C@H]2C#Cc1nn(C)nc1C] | (R)-1-((1,3-dimethyl-1H- pyrazol-4-yl)ethynyl)-4-ethyl- N-(1-methylcyclopropyl)-5- oxo-1,2,4,5- tetrahydroimidazo[1,2- a]quinazoline-7-sulfonamide |
| 266 | 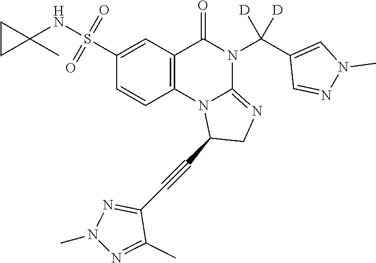 [SMILES: [2H]C([2H])(c1cnn(C)c1)N1C(=O)c2cc(S(=O)(=O)NC3(C)CC3)ccc2N2C1=NC[C@H]2C#Cc1nn(C)nc1C] | (R)-1-((2,5-dimethyl-2H-1,2,3- triazol-4-yl)ethynyl)-4-((1- methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 267 | 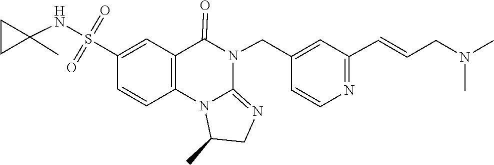 [SMILES: C[C@@H]1CN=C2N(Cc3ccnc(/C=C/CN(C)C)c3)C(=O)c3cc(S(=O)(=O)NC4(C)CC4)ccc3N21] | (R,E)-4-((2-(3- (dimethylamino)prop-1-en-1- yl)pyridin-4-yl)methyl)-1- methyl-N-(1- methylcyclopropyl)-5-oxo- 1,2,4,5-tetrahydroimidazo[1,2- a]quinazoline-7-sulfonamide |
| 268 | 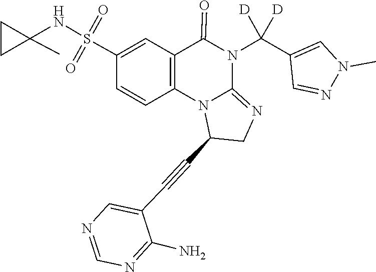 [SMILES: [2H]C([2H])(c1cnn(C)c1)N1C(=O)c2cc(S(=O)(=O)NC3(C)CC3)ccc2N2C1=NC[C@H]2C#Cc1cncnc1N] | (R)-1-((4-aminopyrimidin-5- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 269 | 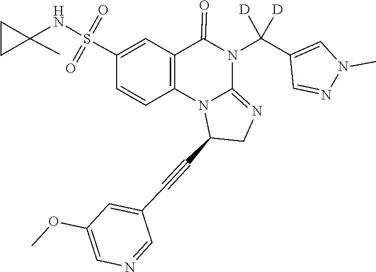 [SMILES: [2H]C([2H])(c1cnn(C)c1)N1C(=O)c2cc(S(=O)(=O)NC3(C)CC3)ccc2N2C1=NC[C@H]2C#Cc1cncc(OC)c1] | (R)-1-((5-methoxypyridin-3- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 270 | 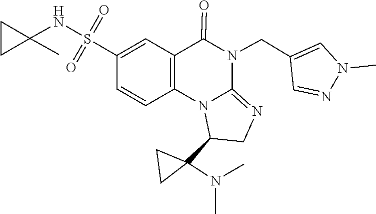 [SMILES: CN(C)C1([C@@H]2CN=C3N(Cc4cnn(C)c4)C(=O)c4cc(S(=O)(=O)NC5(C)CC5)ccc4N32)CC1] | (S)-1-(1- (dimethylamino)cyclopropyl)- 4-((1-methyl-1H-pyrazol-4- yl)methyl)-N-(1- methylcyclopropyl)-5-oxo- 1,2,4,5-tetrahydroimidazo[1,2- a]quinazoline-7-sulfonamide |
| 271 | 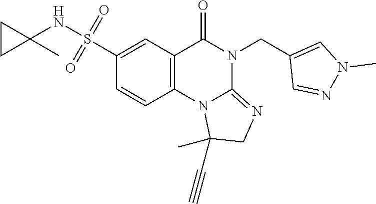 [SMILES: C#CC1(C)CN=C2N(Cc3cnn(C)c3)C(=O)c3cc(S(=O)(=O)NC4(C)CC4)ccc3N21] | 1-ethynyl-1-methyl-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide Absolute stereochemistry not assigned (Isomer A) |
| 272 | 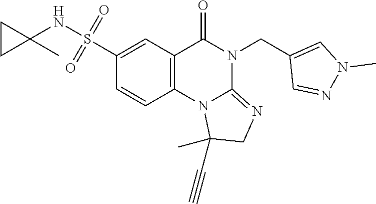 [SMILES: C#CC1(C)CN=C2N(Cc3cnn(C)c3)C(=O)c3cc(S(=O)(=O)NC4(C)CC4)ccc3N21] | 1-ethynyl-1-methyl-4-((1- methyl-1H-pyrazol-4- yl)methyl)-N-(1- methylcyclopropyl)-5-oxo- 1,2,4,5-tetrahydroimidazo[1,2- a]quinazoline-7-sulfonamide Absolute stereochemistry not assigned (Isomer B) |
| 273 | 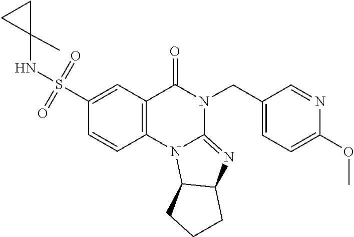 [SMILES: COc1ccc(CN2C(=O)c3cc(S(=O)(=O)NC4(C)CC4)ccc3N3C2=N[C@H]2CCC[C@H]23)cn1] | (7aS,10aR)-6-((6- methoxypyridin-3-yl)methyl)- N-(1-methylcyclopropyl)-5- oxo-6,7a,8,9,10,10a-hexahydro- 5H- cyclopenta[4,5]imidazo[1,2- a]quinazoline-3-sulfonamide |
| 274 | 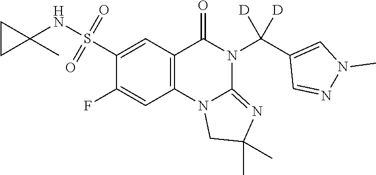 [SMILES: [2H]C([2H])(c1cnn(C)c1)N1C(=O)c2cc(S(=O)(=O)NC3(C)CC3)c(F)cc2N2CC(C)(C)N=C21] | 8-fluoro-2,2-dimethyl-4-((1- methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 275 | 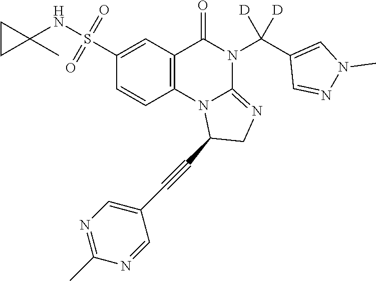 [SMILES: [2H]C([2H])(c1cnn(C)c1)N1C(=O)c2cc(S(=O)(=O)NC3(C)CC3)ccc2N2C1=NC[C@H]2C#Cc1cnc(C)nc1] | (R)-4-((1-methyl-1H-pyrazol-4- yl)methyl-d2)-N-(1- methylcyclopropyl)-1-((2- methylpyrimidin-5-yl)ethynyl)- 5-oxo-1,2,4,5- tetrahydroimidazo[1,2- a]quinazoline-7-sulfonamide |
| 276 | 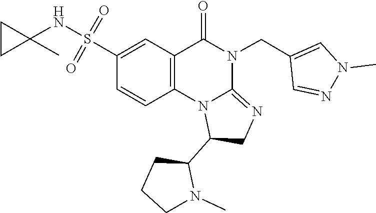 [SMILES: CN1CCC[C@H]1[C@@H]1CN=C2N(Cc3cnn(C)c3)C(=O)c3cc(S(=O)(=O)NC4(C)CC4)ccc3N21] | (S)-4-((1-methyl-1H-pyrazol-4- yl)methyl)-N-(1- methylcyclopropyl)-1-((S)-1- methylpyrrolidin-2-yl)-5-oxo- 1,2,4,5-tetrahydroimidazo[1,2- a]quinazoline-7-sulfonamide |
| 277 | 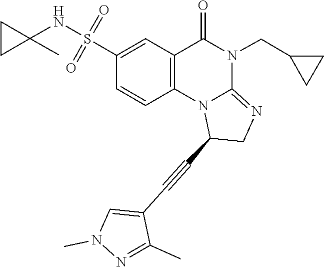 [SMILES: Cc1nn(C)cc1C#C[C@@H]1CN=C2N(CC3CC3)C(=O)c3cc(S(=O)(=O)NC4(C)CC4)ccc3N21] | (R)-4-(cyclopropylmethyl)-1- ((1,3-dimethyl-1H-pyrazol-4- yl)ethynyl)-N-(1- methylcyclopropyl)-5-oxo- 1,2,4,5-tetrahydroimidazo[1,2- a]quinazoline-7-sulfonamide |
| 278 | 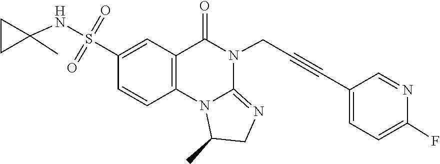 [SMILES: C[C@@H]1CN=C2N(CC#Cc3ccc(F)nc3)C(=O)c3cc(S(=O)(=O)NC4(C)CC4)ccc3N21] | (R)-4-(3-(6-fluoropyridin-3- yl)prop-2-yn-1-yl)-1-methyl-N- (1-methylcyclopropyl)-5-oxo- 1,2,4,5-tetrahydroimidazo[1,2- a]quinazoline-7-sulfonamide |
| 279 | 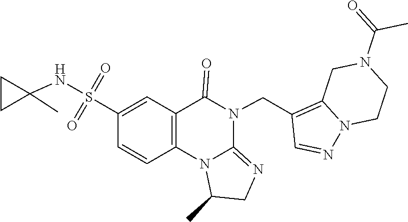 [SMILES: CC(=O)N1CCn2ncc(CN3C(=O)c4cc(S(=O)(=O)NC5(C)CC5)ccc4N4C3=NC[C@H]4C)c2C1] | (R)-4-((5-acetyl-4,5,6,7- tetrahydropyrazolo[1,5- a]pyrazin-3-yl)methyl)-1- methyl-N-(1- methylcyclopropyl)-5-oxo- 1,2,4,5-tetrahydroimidazo[1,2- a]quinazoline-7-sulfonamide |
| 280 | 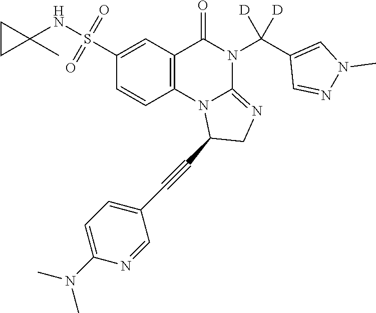 [SMILES: [2H]C([2H])(c1cnn(C)c1)N1C(=O)c2cc(S(=O)(=O)NC3(C)CC3)ccc2N2C1=NC[C@H]2C#Cc1ccc(N(C)C)nc1] | (R)-1-((6- (dimethylamino)pyridin-3- yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 281 | 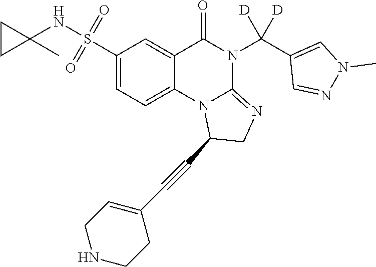 [SMILES: [2H]C([2H])(c1cnn(C)c1)N1C(=O)c2cc(S(=O)(=O)NC3(C)CC3)ccc2N2C1=NC[C@H]2C#CC1=CCNCC1] | (R)-4-((1-methyl-1H-pyrazol-4- yl)methyl-d2)-N-(1- methylcyclopropyl)-5-oxo-1- ((1,2,3,6-tetrahydropyridin-4- yl)ethynyl)-1,2,4,5- tetrahydroimidazo[1,2- a]quinazoline-7-sulfonamide |
| 282 | 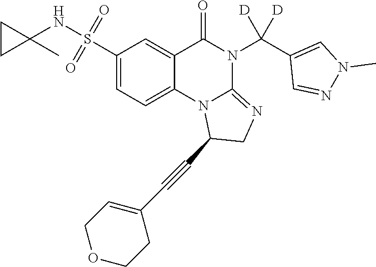 [SMILES: [2H]C([2H])(c1cnn(C)c1)N1C(=O)c2cc(S(=O)(=O)NC3(C)CC3)ccc2N2C1=NC[C@H]2C#CC1=CCOCC1] | (R)-1-((3,6-dihydro-2H-pyran- 4-yl)ethynyl)-4-((1-methyl-1H- pyrazol-4-yl)methyl-d2)-N-(1- methylcyclopropyl)-5-oxo- 1,2,4,5-tetrahydroimidazo[1,2- a]quinazoline-7-sulfonamide |
| 283 | 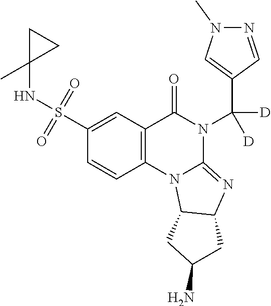 [SMILES: [2H]C([2H])(c1cnn(C)c1)N1C(=O)c2cc(S(=O)(=O)NC3(C)CC3)ccc2N2C1=N[C@@H]1C[C@@H](N)C[C@@H]12] | (7aR,9R,10aS)-9-amino-6-((1- methyl-1H-pyrazol-4- yl)methyl-d2)-N-(1- methylcyclopropyl)-5-oxo- 6,7a,8,9,10,10a-hexahydro-5H- cyclopenta[4,5]imidazo[1,2- a]quinazoline-3-sulfonamide |
| 284 | 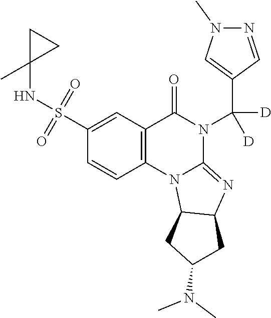 [SMILES: [2H]C([2H])(c1cnn(C)c1)N1C(=O)c2cc(S(=O)(=O)NC3(C)CC3)ccc2N2C1=N[C@H]1C[C@H](N(C)C)C[C@H]12] | (7aS,9S,10aR)-9- (dimethylamino)-6-((1-methyl- 1H-pyrazol-4-yl)methyl-d2)-N- (1-methylcyclopropyl)-5-oxo- 6,7a,8,9,10,10a-hexahydro-5H- cyclopenta[4,5]imidazo[1,2- a]quinazoline-3-sulfonamide |
| 285 | 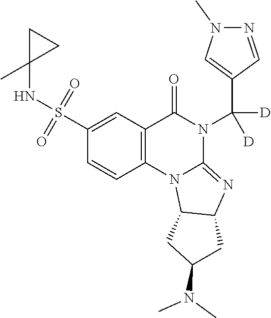 [SMILES: [2H]C([2H])(c1cnn(C)c1)N1C(=O)c2cc(S(=O)(=O)NC3(C)CC3)ccc2N2C1=N[C@@H]1C[C@@H](N(C)C)C[C@@H]12] | (7aR,9R,10aS)-9- (dimethylamino)-6-((1-methyl- 1H-pyrazol-4-yl)methyl-d2)-N- (1-methylcyclopropyl)-5-oxo- 6,7a,8,9,10,10a-hexahydro-5H- cyclopenta[4,5]imidazo[1,2- a]quinazoline-3-sulfonamide |
| 286 | 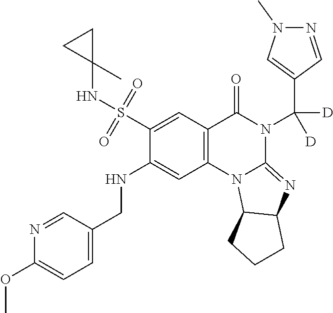 [SMILES: [2H]C([2H])(c1cnn(C)c1)N1C(=O)c2cc(S(=O)(=O)NC3(C)CC3)c(NCc3ccc(OC)nc3)cc2N2C1=N[C@H]1CCC[C@H]12] | (7aS,10aR)-2-(((6- methoxypyridin-3- yl)methyl)amino)-6-((1-methyl- 1H-pyrazol-4-yl)methyl-d2)-N- (1-methylcyclopropyl)-5-oxo- 6,7a,8,9,10,10a-hexahydro-5H- cyclopenta[4,5]imidazo[1,2- a]quinazoline-3-sulfonamide |
| 287 | 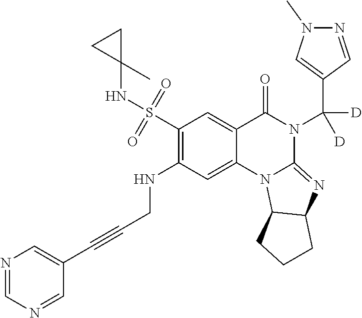 [SMILES: [2H]C([2H])(c1cnn(C)c1)N1C(=O)c2cc(S(=O)(=O)NC3(C)CC3)c(NCC#Cc3cncnc3)cc2N2C1=N[C@H]1CCC[C@H]12] | (7aS,10aR)-6-((1-methyl-1H- pyrazol-4-yl)methyl-d2)-N-(1- methylcyclopropyl)-5-oxo-2- ((3-(pyrimidin-5-yl)prop-2-yn- 1-yl)amino)-6,7a,8,9,10,10a- hexahydro-5H- cyclopenta[4,5]imidazo[1,2- a]quinazoline-3-sulfonamide |
| 288 | 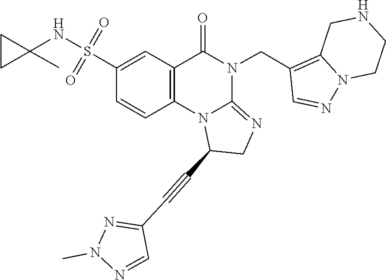 [SMILES: Cn1ncc(C#C[C@@H]2CN=C3N(Cc4cnn5c4CNCC5)C(=O)c4cc(S(=O)(=O)NC5(C)CC5)ccc4N32)n1] | (R)-1-((2-methyl-2H-1,2,3- triazol-4-yl)ethynyl)-N-(1- methylcyclopropyl)-5-oxo-4- ((4,5,6,7- tetrahydropyrazolo[1,5- a]pyrazin-3-yl)methyl)-1,2,4,5- tetrahydroimidazo[1,2- a]quinazoline-7-sulfonamide |
| 289 | 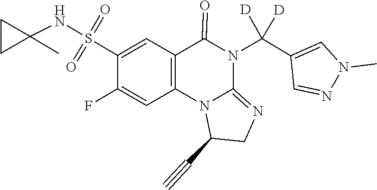 [SMILES: [2H]C([2H])(c1cnn(C)c1)N1C(=O)c2cc(S(=O)(=O)NC3(C)CC3)c(F)cc2N2C1=NC[C@H]2C#C] | (R)-1-ethynyl-8-fluoro-4-((1- methyl-1H-pyrazol-4- yl)methyl-d2)-N-(1- methylcyclopropyl)-5-oxo- 1,2,4,5-tetrahydroimidazo[1,2- a]quinazoline-7-sulfonamide |
| 290 | 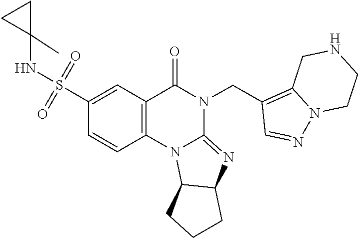 [SMILES: CC1(NS(=O)(=O)c2ccc3c(c2)C(=O)N(Cc2cnn4c2CNCC4)C2=N[C@H]4CCC[C@H]4N23)CC1] | (7aS,10aR)-N-(1- methylcyclopropyl)-5-oxo-6- ((4,5,6,7- tetrahydropyrazolo[1,5- a]pyrazin-3-yl)methyl)- 6,7a,8,9,10,10a-hexahydro-5H- cyclopenta[4,5]imidazo[1,2- a]quinazoline-3-sulfonamide |
| 291 | 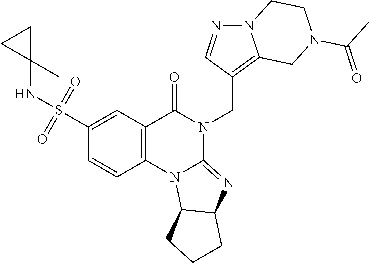 [SMILES: CC(=O)N1CCn2ncc(CN3C(=O)c4cc(S(=O)(=O)NC5(C)CC5)ccc4N4C3=N[C@H]3CCC[C@H]34)c2C1] | (7aS,10aR)-6-((5-acetyl- 4,5,6,7-tetrahydropyrazolo[1,5- a]pyrazin-3-yl)methyl)-N-(1- methylcyclopropyl)-5-oxo- 6,7a,8,9,10,10a-hexahydro-5H- cyclopenta[4,5]imidazo[1,2- a]quinazoline-3-sulfonamide |
| 292 | 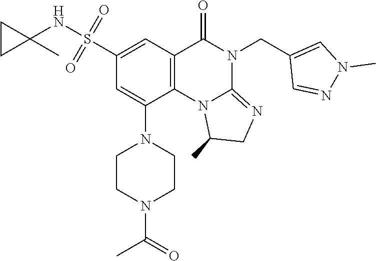 [SMILES: CC(=O)N1CCN(c2cc(S(=O)(=O)NC3(C)CC3)cc3c2N2C(=NC[C@H]2C)N(Cc2cnn(C)c2)C3=O)CC1] | (R)-9-(4-acetylpiperazin-1-yl)- 1-methyl-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 293 | 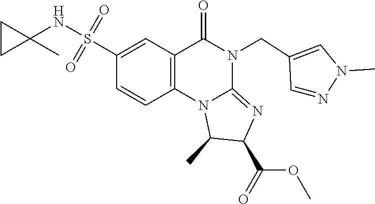 [SMILES: COC(=O)[C@@H]1N=C2N(Cc3cnn(C)c3)C(=O)c3cc(S(=O)(=O)NC4(C)CC4)ccc3N2[C@@H]1C] | methyl (1R,2R)-1-methyl-4- ((1-methyl-1H-pyrazol-4- yl)methyl)-7-(N-(1- methylcyclopropyl)sulfamoyl)- 5-oxo-1,2,4,5- tetrahydroimidazo[1,2- a]quinazoline-2-carboxylate |
| 294 | 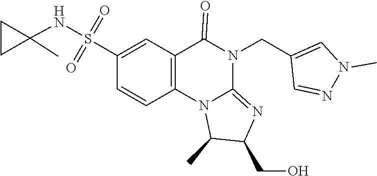 [SMILES: C[C@@H]1[C@H](CO)N=C2N(Cc3cnn(C)c3)C(=O)c3cc(S(=O)(=O)NC4(C)CC4)ccc3N21] | (1R,2R)-2-(hydroxymethyl)-1- methyl-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 295 | 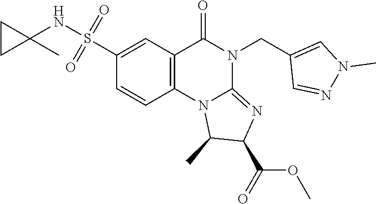 [SMILES: COC(=O)[C@@H]1N=C2N(Cc3cnn(C)c3)C(=O)c3cc(S(=O)(=O)NC4(C)CC4)ccc3N2[C@@H]1C] | N-(((1R,2S)-1-methyl-4-((1- methyl-1H-pyrazol-4- yl)methyl)-7-(N-(1- methylcyclopropyl)sulfamoyl)- 5-oxo-1,2,4,5- tetrahydroimidazo[1,2- a]quinazolin-2- yl)methyl)acetamide |
| 296 | 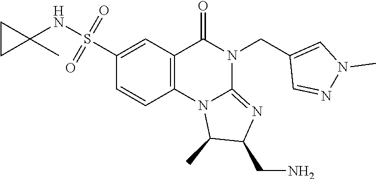 [SMILES: C[C@@H]1[C@H](CN)N=C2N(Cc3cnn(C)c3)C(=O)c3cc(S(=O)(=O)NC4(C)CC4)ccc3N21] | (1R,2S)-2-(aminomethyl)-1- methyl-4-((1-methyl-1H- pyrazol-4-yl)methyl)-N-(1- methylcyclopropyl)-5-oxo- 1,2,4,5-tetrahydroimidazo[1,2- a]quinazoline-7-sulfonamide |
| 297 | 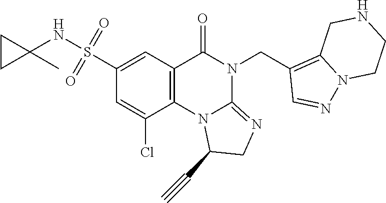 [SMILES: C#C[C@@H]1CN=C2N(Cc3cnn4c3CNCC4)C(=O)c3cc(S(=O)(=O)NC4(C)CC4)cc(Cl)c3N21] | (R)-9-chloro-1-ethynyl-N-(1- methylcyclopropyl)-5-oxo-4- ((4,5,6,7- tetrahydropyrazolo[1,5- a]pyrazin-3-yl)methyl)-1,2,4,5- tetrahydroimidazo[1,2- a]quinazoline-7-sulfonamide |

Another embodiment provides an PARG inhibitory compound, or a pharmaceutically acceptable salt or solvate thereof, having a structure presented in Tables 2A-2B.

TABLE 2A

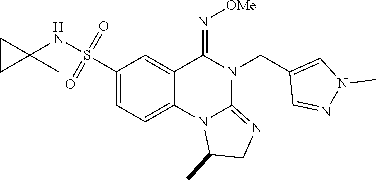 [SMILES: CO/N=C1/c2cc(S(=O)(=O)NC3(C)CC3)ccc2N2C(=NC[C@H]2C)N1Cc1cnn(C)c1]

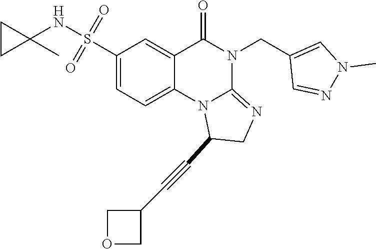 [SMILES: Cn1cc(CN2C(=O)c3cc(S(=O)(=O)NC4(C)CC4)ccc3N3C2=NC[C@H]3C#CC2COC2)cn1]

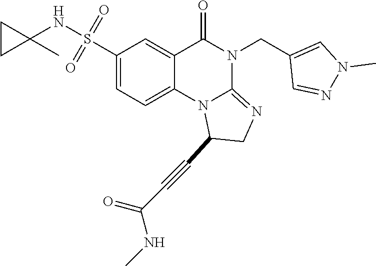 [SMILES: CNC(=O)C#C[C@@H]1CN=C2N(Cc3cnn(C)c3)C(=O)c3cc(S(=O)(=O)NC4(C)CC4)ccc3N21]

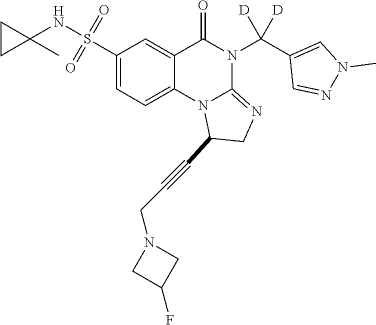 [SMILES: [2H]C([2H])(c1cnn(C)c1)N1C(=O)c2cc(S(=O)(=O)NC3(C)CC3)ccc2N2C1=NC[C@H]2C#CCN1CC(F)C1]

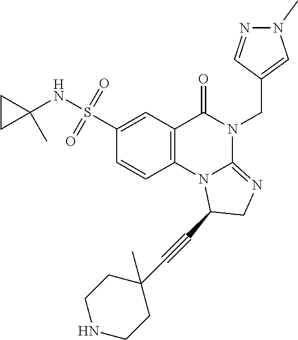 [SMILES: Cn1cc(CN2C(=O)c3cc(S(=O)(=O)NC4(C)CC4)ccc3N3C2=NC[C@H]3C#CC2(C)CCNCC2)cn1]

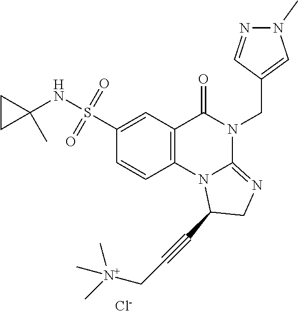 [SMILES: Cn1cc(CN2C(=O)c3cc(S(=O)(=O)NC4(C)CC4)ccc3N3C2=NC[C@H]3C#CC[N+](C)(C)C)cn1 ; [Cl-]]

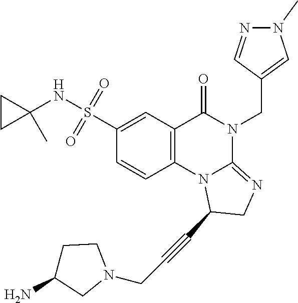 [SMILES: Cn1cc(CN2C(=O)c3cc(S(=O)(=O)NC4(C)CC4)ccc3N3C2=NC[C@H]3C#CCN2CC[C@H](N)C2)cn1]

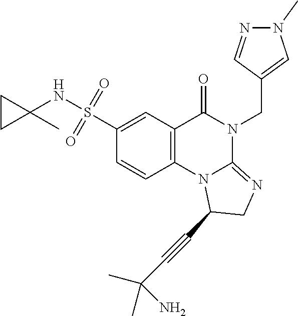 [SMILES: Cn1cc(CN2C(=O)c3cc(S(=O)(=O)NC4(C)CC4)ccc3N3C2=NC[C@H]3C#CC(C)(C)N)cn1]

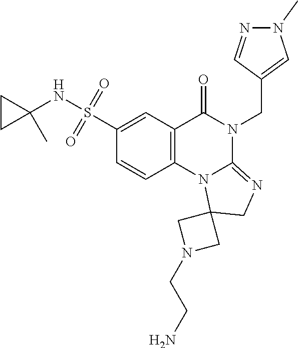 [SMILES: Cn1cc(CN2C(=O)c3cc(S(=O)(=O)NC4(C)CC4)ccc3N3C2=NCC32CN(CCN)C2)cn1]

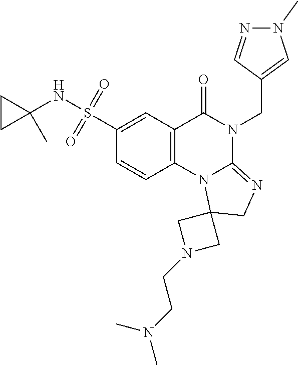 [SMILES: CN(C)CCN1CC2(CN=C3N(Cc4cnn(C)c4)C(=O)c4cc(S(=O)(=O)NC5(C)CC5)ccc4N32)C1]

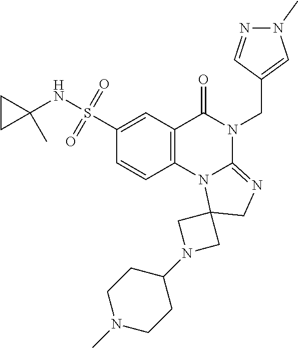 [SMILES: CN1CCC(N2CC3(CN=C4N(Cc5cnn(C)c5)C(=O)c5cc(S(=O)(=O)NC6(C)CC6)ccc5N43)C2)CC1]

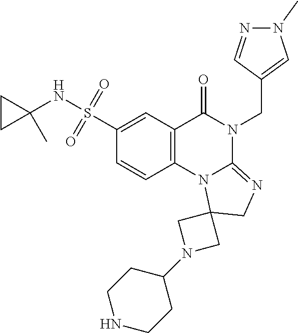 [SMILES: Cn1cc(CN2C(=O)c3cc(S(=O)(=O)NC4(C)CC4)ccc3N3C2=NCC32CN(C3CCNCC3)C2)cn1]

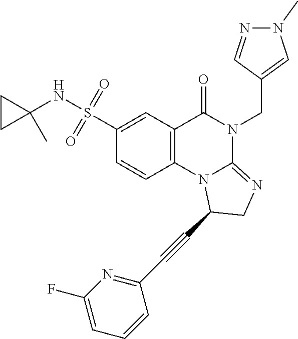 [SMILES: Cn1cc(CN2C(=O)c3cc(S(=O)(=O)NC4(C)CC4)ccc3N3C2=NC[C@H]3C#Cc2cccc(F)n2)cn1]

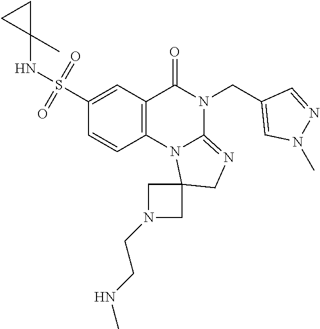 [SMILES: CNCCN1CC2(CN=C3N(Cc4cnn(C)c4)C(=O)c4cc(S(=O)(=O)NC5(C)CC5)ccc4N32)C1]

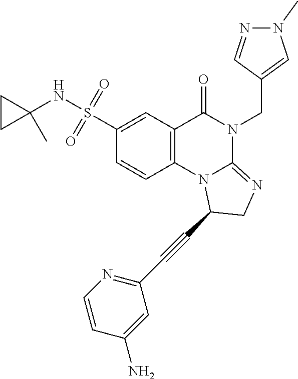 [SMILES: Cn1cc(CN2C(=O)c3cc(S(=O)(=O)NC4(C)CC4)ccc3N3C2=NC[C@H]3C#Cc2cc(N)ccn2)cn1]

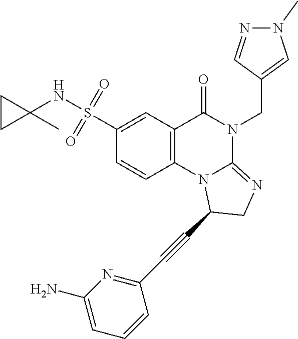 [SMILES: Cn1cc(CN2C(=O)c3cc(S(=O)(=O)NC4(C)CC4)ccc3N3C2=NC[C@H]3C#Cc2cccc(N)n2)cn1]

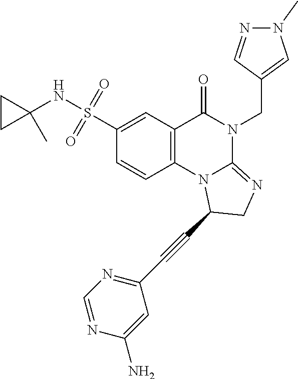 [SMILES: Cn1cc(CN2C(=O)c3cc(S(=O)(=O)NC4(C)CC4)ccc3N3C2=NC[C@H]3C#Cc2cc(N)ncn2)cn1]

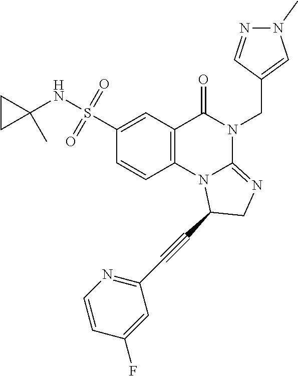 [SMILES: Cn1cc(CN2C(=O)c3cc(S(=O)(=O)NC4(C)CC4)ccc3N3C2=NC[C@H]3C#Cc2cc(F)ccn2)cn1]

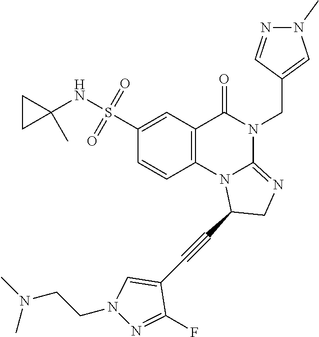

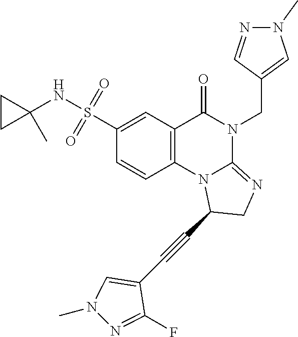 [SMILES: Cn1cc(CN2C(=O)c3cc(S(=O)(=O)NC4(C)CC4)ccc3N3C2=NC[C@H]3C#Cc2cn(C)nc2F)cn1]

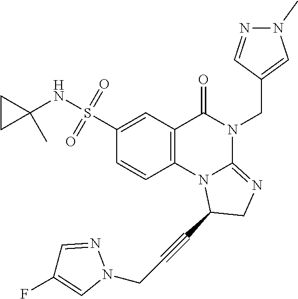 [SMILES: Cn1cc(CN2C(=O)c3cc(S(=O)(=O)NC4(C)CC4)ccc3N3C2=NC[C@H]3C#CCn2cc(F)cn2)cn1]

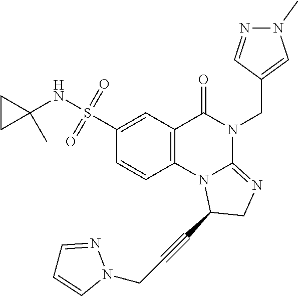 [SMILES: Cn1cc(CN2C(=O)c3cc(S(=O)(=O)NC4(C)CC4)ccc3N3C2=NC[C@H]3C#CCn2cccn2)cn1]

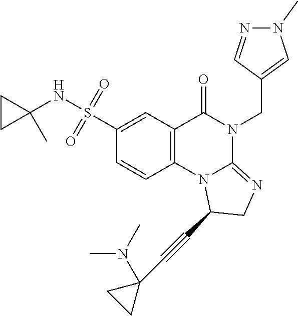 [SMILES: CN(C)C1(C#C[C@@H]2CN=C3N(Cc4cnn(C)c4)C(=O)c4cc(S(=O)(=O)NC5(C)CC5)ccc4N32)CC1]

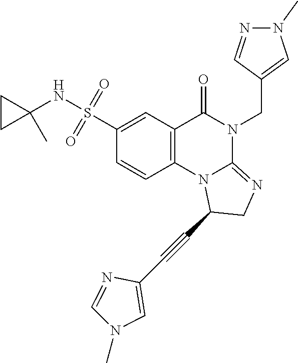 [SMILES: Cn1cnc(C#C[C@@H]2CN=C3N(Cc4cnn(C)c4)C(=O)c4cc(S(=O)(=O)NC5(C)CC5)ccc4N32)c1]

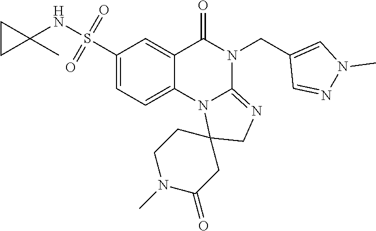 [SMILES: CN1CCC2(CN=C3N(Cc4cnn(C)c4)C(=O)c4cc(S(=O)(=O)NC5(C)CC5)ccc4N32)CC1=O]

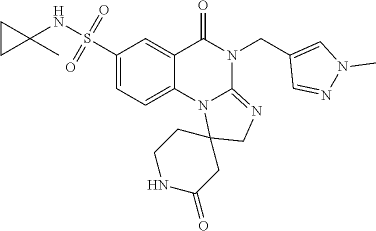 [SMILES: Cn1cc(CN2C(=O)c3cc(S(=O)(=O)NC4(C)CC4)ccc3N3C2=NCC32CCNC(=O)C2)cn1]

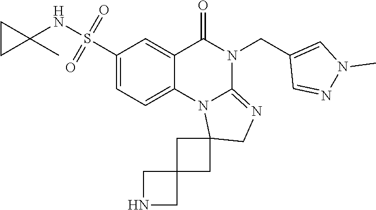 [SMILES: Cn1cc(CN2C(=O)c3cc(S(=O)(=O)NC4(C)CC4)ccc3N3C2=NCC32CC3(CNC3)C2)cn1]

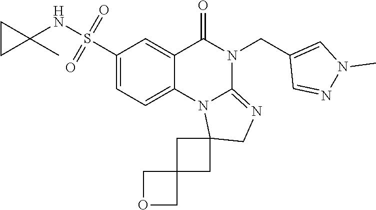 [SMILES: Cn1cc(CN2C(=O)c3cc(S(=O)(=O)NC4(C)CC4)ccc3N3C2=NCC32CC3(COC3)C2)cn1]

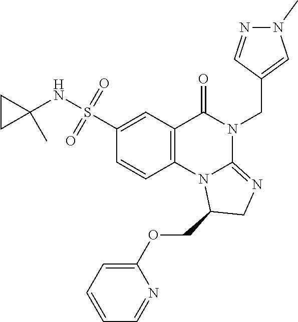 [SMILES: Cn1cc(CN2C(=O)c3cc(S(=O)(=O)NC4(C)CC4)ccc3N3C2=NC[C@H]3COc2ccccn2)cn1]

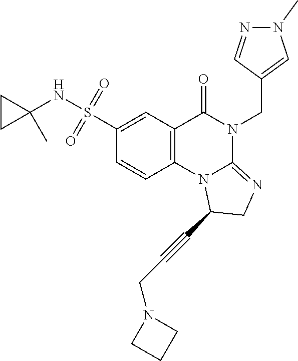 [SMILES: Cn1cc(CN2C(=O)c3cc(S(=O)(=O)NC4(C)CC4)ccc3N3C2=NC[C@H]3C#CCN2CCC2)cn1]

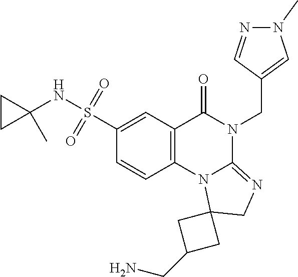 [SMILES: Cn1cc(CN2C(=O)c3cc(S(=O)(=O)NC4(C)CC4)ccc3N3C2=NCC32CC(CN)C2)cn1]

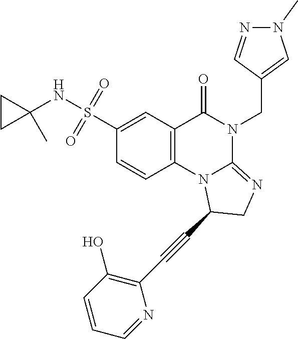 [SMILES: Cn1cc(CN2C(=O)c3cc(S(=O)(=O)NC4(C)CC4)ccc3N3C2=NC[C@H]3C#Cc2ncccc2O)cn1]

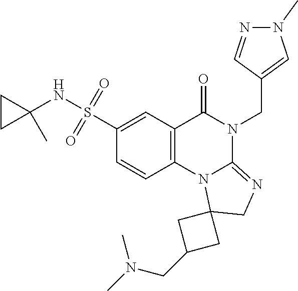 [SMILES: CN(C)CC1CC2(CN=C3N(Cc4cnn(C)c4)C(=O)c4cc(S(=O)(=O)NC5(C)CC5)ccc4N32)C1]

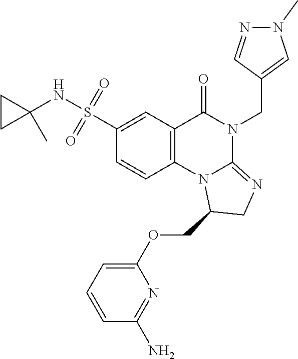 [SMILES: Cn1cc(CN2C(=O)c3cc(S(=O)(=O)NC4(C)CC4)ccc3N3C2=NC[C@H]3COc2cccc(N)n2)cn1]

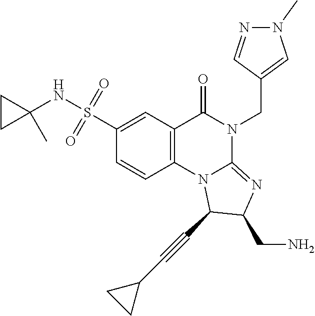 [SMILES: Cn1cc(CN2C(=O)c3cc(S(=O)(=O)NC4(C)CC4)ccc3N3C2=N[C@@H](CN)[C@H]3C#CC2CC2)cn1]

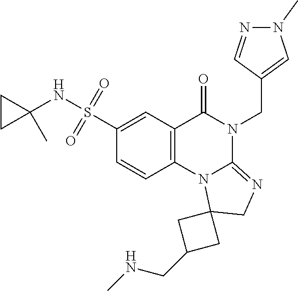 [SMILES: CNCC1CC2(CN=C3N(Cc4cnn(C)c4)C(=O)c4cc(S(=O)(=O)NC5(C)CC5)ccc4N32)C1]

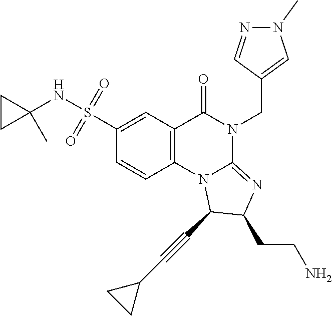 [SMILES: Cn1cc(CN2C(=O)c3cc(S(=O)(=O)NC4(C)CC4)ccc3N3C2=N[C@@H](CCN)[C@H]3C#CC2CC2)cn1]

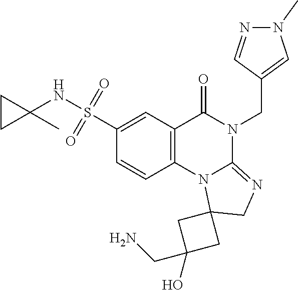 [SMILES: Cn1cc(CN2C(=O)c3cc(S(=O)(=O)NC4(C)CC4)ccc3N3C2=NCC32CC(O)(CN)C2)cn1]

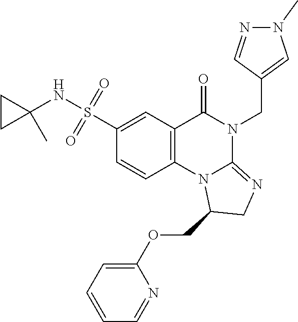 [SMILES: Cn1cc(CN2C(=O)c3cc(S(=O)(=O)NC4(C)CC4)ccc3N3C2=NC[C@H]3COc2ccccn2)cn1]

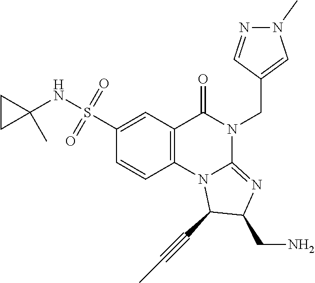 [SMILES: CC#C[C@@H]1[C@H](CN)N=C2N(Cc3cnn(C)c3)C(=O)c3cc(S(=O)(=O)NC4(C)CC4)ccc3N21]

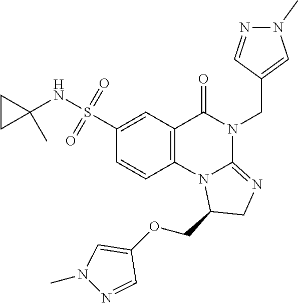 [SMILES: Cn1cc(CN2C(=O)c3cc(S(=O)(=O)NC4(C)CC4)ccc3N3C2=NC[C@H]3COc2cnn(C)c2)cn1]

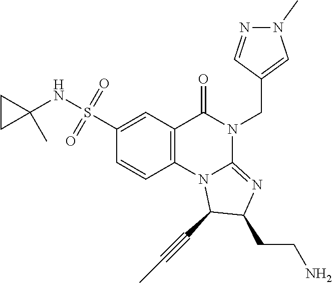 [SMILES: CC#C[C@@H]1[C@H](CCN)N=C2N(Cc3cnn(C)c3)C(=O)c3cc(S(=O)(=O)NC4(C)CC4)ccc3N21]

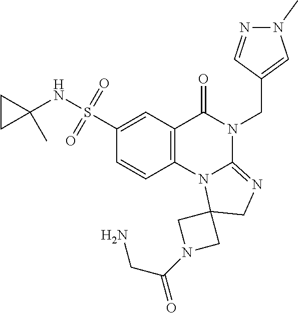 [SMILES: Cn1cc(CN2C(=O)c3cc(S(=O)(=O)NC4(C)CC4)ccc3N3C2=NCC32CN(C(=O)CN)C2)cn1]

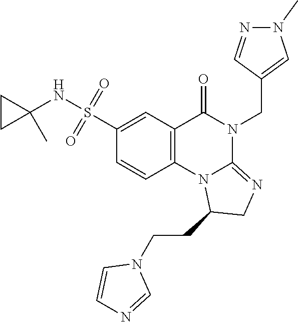 [SMILES: Cn1cc(CN2C(=O)c3cc(S(=O)(=O)NC4(C)CC4)ccc3N3C2=NC[C@H]3CCn2ccnc2)cn1]

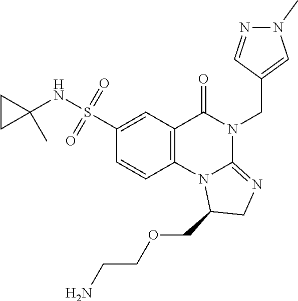 [SMILES: Cn1cc(CN2C(=O)c3cc(S(=O)(=O)NC4(C)CC4)ccc3N3C2=NC[C@H]3COCCN)cn1]

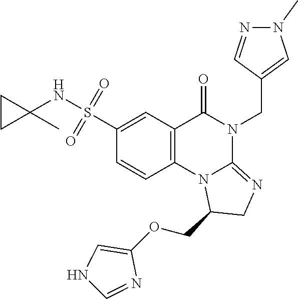 [SMILES: Cn1cc(CN2C(=O)c3cc(S(=O)(=O)NC4(C)CC4)ccc3N3C2=NC[C@H]3COc2c[nH]cn2)cn1]

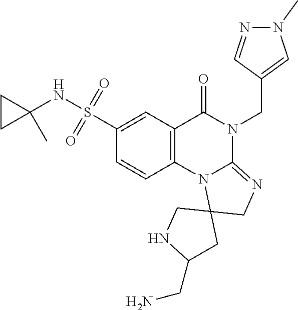 [SMILES: Cn1cc(CN2C(=O)c3cc(S(=O)(=O)NC4(C)CC4)ccc3N3C2=NCC32CNC(CN)C2)cn1]

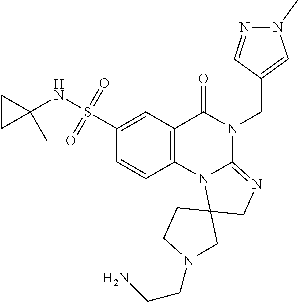 [SMILES: Cn1cc(CN2C(=O)c3cc(S(=O)(=O)NC4(C)CC4)ccc3N3C2=NCC32CCN(CCN)C2)cn1]

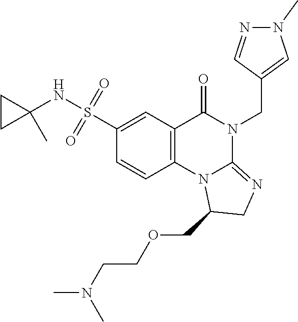 [SMILES: CN(C)CCOC[C@@H]1CN=C2N(Cc3cnn(C)c3)C(=O)c3cc(S(=O)(=O)NC4(C)CC4)ccc3N21]

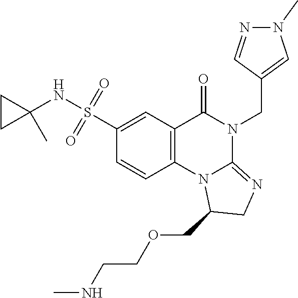 [SMILES: CNCCOC[C@@H]1CN=C2N(Cc3cnn(C)c3)C(=O)c3cc(S(=O)(=O)NC4(C)CC4)ccc3N21]

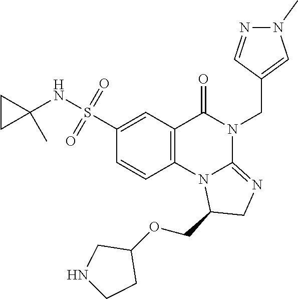 [SMILES: Cn1cc(CN2C(=O)c3cc(S(=O)(=O)NC4(C)CC4)ccc3N3C2=NC[C@H]3COC2CCNC2)cn1]

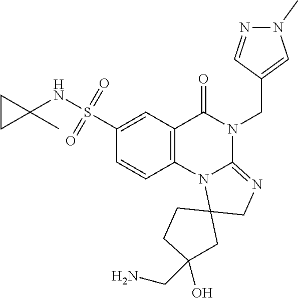 [SMILES: Cn1cc(CN2C(=O)c3cc(S(=O)(=O)NC4(C)CC4)ccc3N3C2=NCC32CCC(O)(CN)C2)cn1]

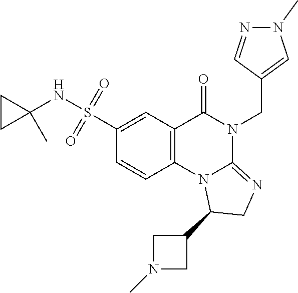 [SMILES: CN1CC([C@@H]2CN=C3N(Cc4cnn(C)c4)C(=O)c4cc(S(=O)(=O)NC5(C)CC5)ccc4N32)C1]

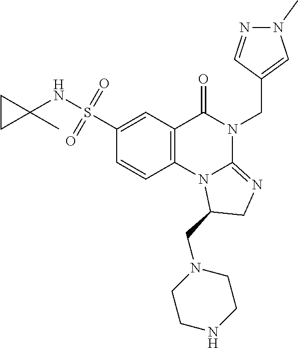 [SMILES: Cn1cc(CN2C(=O)c3cc(S(=O)(=O)NC4(C)CC4)ccc3N3C2=NC[C@H]3CN2CCNCC2)cn1]

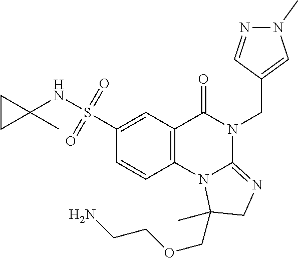 [SMILES: Cn1cc(CN2C(=O)c3cc(S(=O)(=O)NC4(C)CC4)ccc3N3C2=NCC3(C)COCCN)cn1]

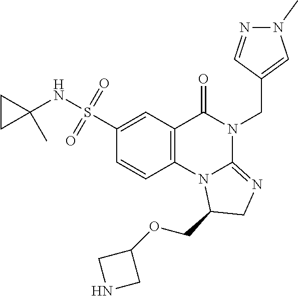 [SMILES: Cn1cc(CN2C(=O)c3cc(S(=O)(=O)NC4(C)CC4)ccc3N3C2=NC[C@H]3COC2CNC2)cn1]

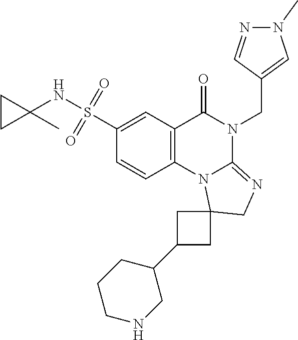 [SMILES: Cn1cc(CN2C(=O)c3cc(S(=O)(=O)NC4(C)CC4)ccc3N3C2=NCC32CC(C3CCCNC3)C2)cn1]

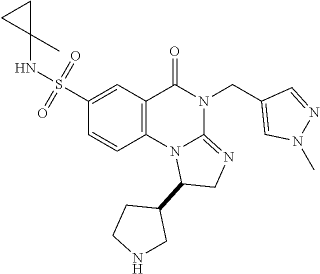 [SMILES: Cn1cc(CN2C(=O)c3cc(S(=O)(=O)NC4(C)CC4)ccc3N3C2=NC[C@H]3C2CCNC2)cn1]

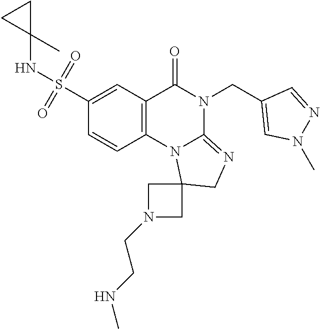 [SMILES: CNCCN1CC2(CN=C3N(Cc4cnn(C)c4)C(=O)c4cc(S(=O)(=O)NC5(C)CC5)ccc4N32)C1]

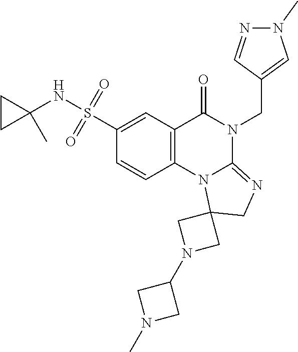 [SMILES: CN1CC(N2CC3(CN=C4N(Cc5cnn(C)c5)C(=O)c5cc(S(=O)(=O)NC6(C)CC6)ccc5N43)C2)C1]

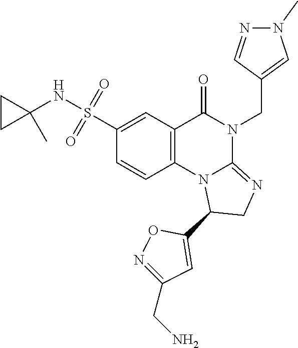 [SMILES: Cn1cc(CN2C(=O)c3cc(S(=O)(=O)NC4(C)CC4)ccc3N3C2=NC[C@H]3c2cc(CN)no2)cn1]

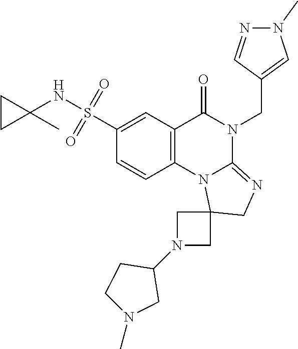 [SMILES: CN1CCC(N2CC3(CN=C4N(Cc5cnn(C)c5)C(=O)c5cc(S(=O)(=O)NC6(C)CC6)ccc5N43)C2)C1]

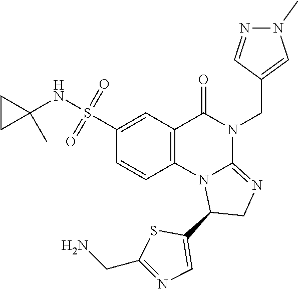 [SMILES: Cn1cc(CN2C(=O)c3cc(S(=O)(=O)NC4(C)CC4)ccc3N3C2=NC[C@H]3c2cnc(CN)s2)cn1]

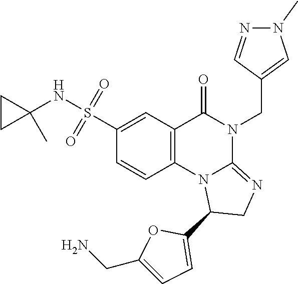 [SMILES: Cn1cc(CN2C(=O)c3cc(S(=O)(=O)NC4(C)CC4)ccc3N3C2=NC[C@H]3c2ccc(CN)o2)cn1]

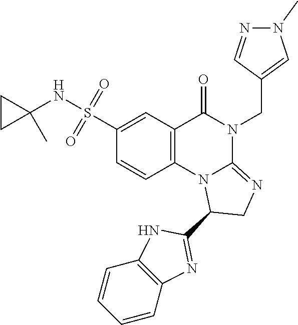 [SMILES: Cn1cc(CN2C(=O)c3cc(S(=O)(=O)NC4(C)CC4)ccc3N3C2=NC[C@H]3c2nc3ccccc3[nH]2)cn1]

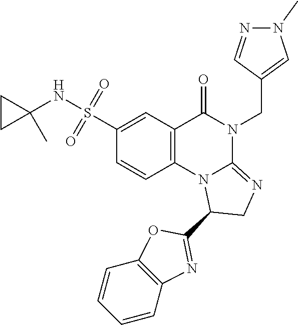 [SMILES: Cn1cc(CN2C(=O)c3cc(S(=O)(=O)NC4(C)CC4)ccc3N3C2=NC[C@H]3c2nc3ccccc3o2)cn1]

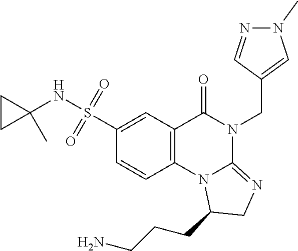 [SMILES: Cn1cc(CN2C(=O)c3cc(S(=O)(=O)NC4(C)CC4)ccc3N3C2=NC[C@H]3CCCN)cn1]

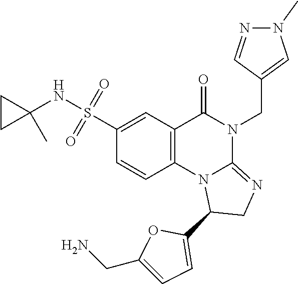 [SMILES: Cn1cc(CN2C(=O)c3cc(S(=O)(=O)NC4(C)CC4)ccc3N3C2=NC[C@H]3c2ccc(CN)o2)cn1]

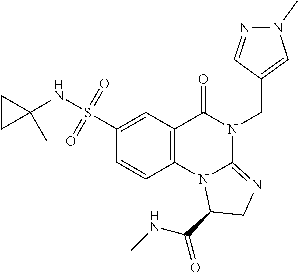 [SMILES: CNC(=O)[C@@H]1CN=C2N(Cc3cnn(C)c3)C(=O)c3cc(S(=O)(=O)NC4(C)CC4)ccc3N21]

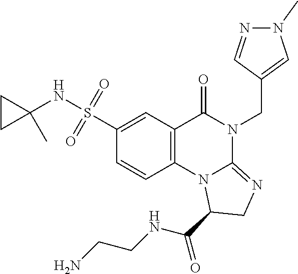 [SMILES: Cn1cc(CN2C(=O)c3cc(S(=O)(=O)NC4(C)CC4)ccc3N3C2=NC[C@H]3C(=O)NCCN)cn1]

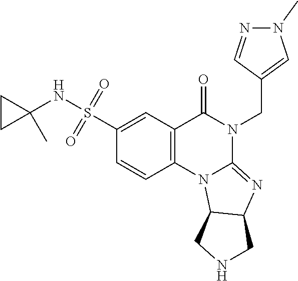 [SMILES: Cn1cc(CN2C(=O)c3cc(S(=O)(=O)NC4(C)CC4)ccc3N3C2=N[C@H]2CNC[C@H]23)cn1]

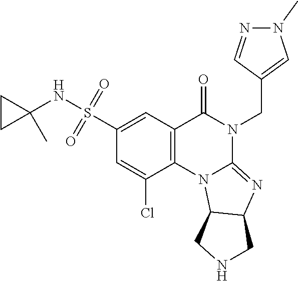 [SMILES: Cn1cc(CN2C(=O)c3cc(S(=O)(=O)NC4(C)CC4)cc(Cl)c3N3C2=N[C@H]2CNC[C@H]23)cn1]

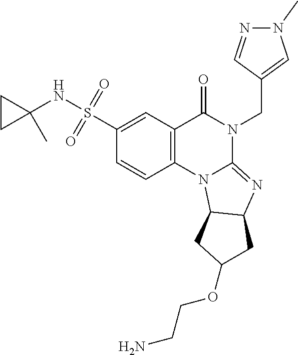

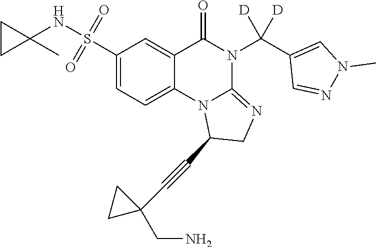 [SMILES: [2H]C([2H])(c1cnn(C)c1)N1C(=O)c2cc(S(=O)(=O)NC3(C)CC3)ccc2N2C1=NC[C@H]2C#CC1(CN)CC1]

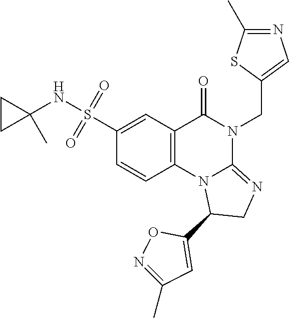 [SMILES: Cc1cc([C@@H]2CN=C3N(Cc4cnc(C)s4)C(=O)c4cc(S(=O)(=O)NC5(C)CC5)ccc4N32)on1]

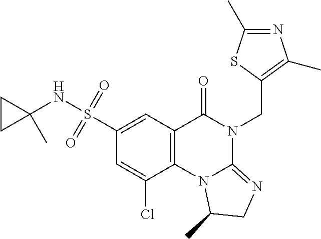 [SMILES: Cc1nc(C)c(CN2C(=O)c3cc(S(=O)(=O)NC4(C)CC4)cc(Cl)c3N3C2=NC[C@H]3C)s1]

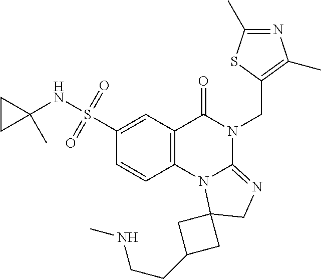 [SMILES: CNCCC1CC2(CN=C3N(Cc4sc(C)nc4C)C(=O)c4cc(S(=O)(=O)NC5(C)CC5)ccc4N32)C1]

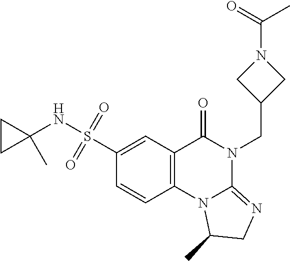 [SMILES: CC(=O)N1CC(CN2C(=O)c3cc(S(=O)(=O)NC4(C)CC4)ccc3N3C2=NC[C@H]3C)C1]

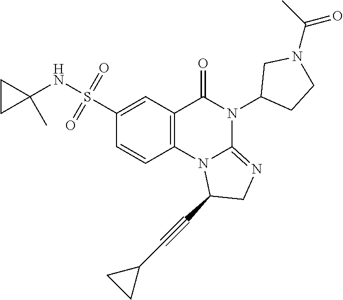 [SMILES: CC(=O)N1CCC(N2C(=O)c3cc(S(=O)(=O)NC4(C)CC4)ccc3N3C2=NC[C@H]3C#CC2CC2)C1]

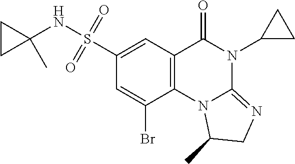 [SMILES: C[C@@H]1CN=C2N(C3CC3)C(=O)c3cc(S(=O)(=O)NC4(C)CC4)cc(Br)c3N21]

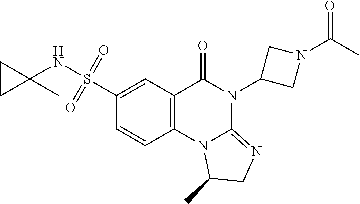 [SMILES: CC(=O)N1CC(N2C(=O)c3cc(S(=O)(=O)NC4(C)CC4)ccc3N3C2=NC[C@H]3C)C1]

—

TABLE 2B

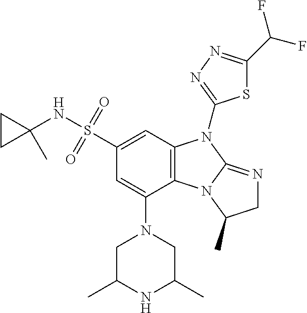 [SMILES: CC1CN(c2cc(S(=O)(=O)NC3(C)CC3)cc3c2N2C(=NC[C@H]2C)N3c2nnc(C(F)F)s2)CC(C)N1]

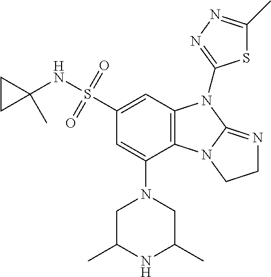 [SMILES: Cc1nnc(N2C3=NCCN3c3c(N4CC(C)NC(C)C4)cc(S(=O)(=O)NC4(C)CC4)cc32)s1]

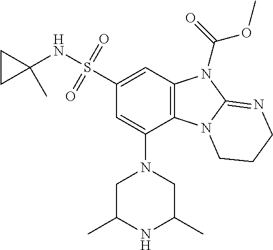 [SMILES: COC(=O)N1C2=NCCCN2c2c(N3CC(C)NC(C)C3)cc(S(=O)(=O)NC3(C)CC3)cc21]

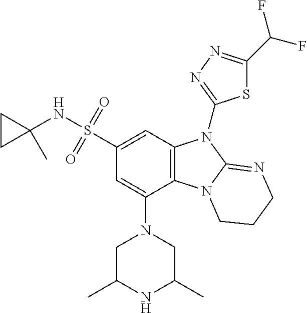 [SMILES: CC1CN(c2cc(S(=O)(=O)NC3(C)CC3)cc3c2N2CCCN=C2N3c2nnc(C(F)F)s2)CC(C)N1]

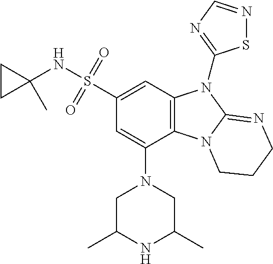 [SMILES: CC1CN(c2cc(S(=O)(=O)NC3(C)CC3)cc3c2N2CCCN=C2N3c2ncns2)CC(C)N1]

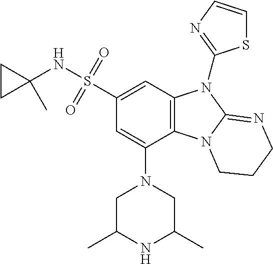 [SMILES: CC1CN(c2cc(S(=O)(=O)NC3(C)CC3)cc3c2N2CCCN=C2N3c2nccs2)CC(C)N1]

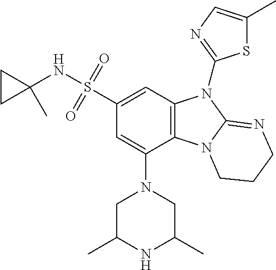 [SMILES: Cc1cnc(N2C3=NCCCN3c3c(N4CC(C)NC(C)C4)cc(S(=O)(=O)NC4(C)CC4)cc32)s1]

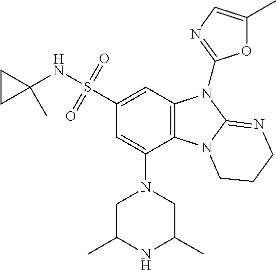 [SMILES: Cc1cnc(N2C3=NCCCN3c3c(N4CC(C)NC(C)C4)cc(S(=O)(=O)NC4(C)CC4)cc32)o1]

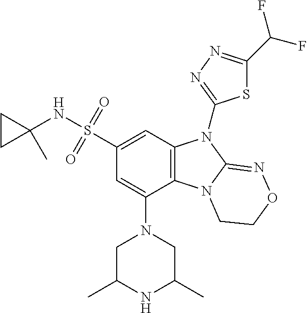 [SMILES: CC1CN(c2cc(S(=O)(=O)NC3(C)CC3)cc3c2N2CCON=C2N3c2nnc(C(F)F)s2)CC(C)N1]

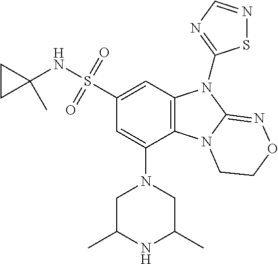 [SMILES: CC1CN(c2cc(S(=O)(=O)NC3(C)CC3)cc3c2N2CCON=C2N3c2ncns2)CC(C)N1]

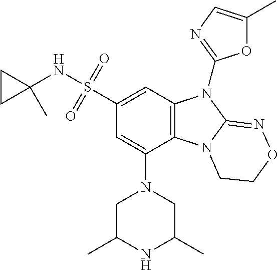 [SMILES: Cc1cnc(N2C3=NOCCN3c3c(N4CC(C)NC(C)C4)cc(S(=O)(=O)NC4(C)CC4)cc32)o1]

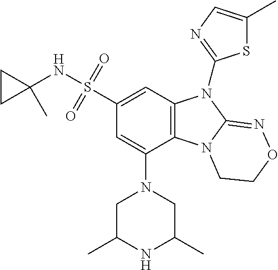 [SMILES: Cc1cnc(N2C3=NOCCN3c3c(N4CC(C)NC(C)C4)cc(S(=O)(=O)NC4(C)CC4)cc32)s1]

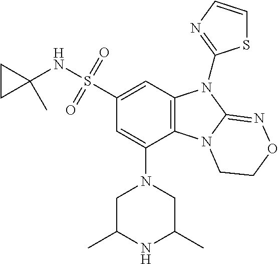 [SMILES: CC1CN(c2cc(S(=O)(=O)NC3(C)CC3)cc3c2N2CCON=C2N3c2nccs2)CC(C)N1]

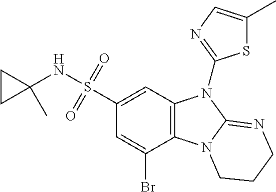 [SMILES: Cc1cnc(N2C3=NCCCN3c3c(Br)cc(S(=O)(=O)NC4(C)CC4)cc32)s1]

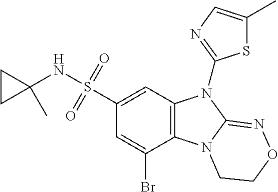 [SMILES: Cc1cnc(N2C3=NOCCN3c3c(Br)cc(S(=O)(=O)NC4(C)CC4)cc32)s1]

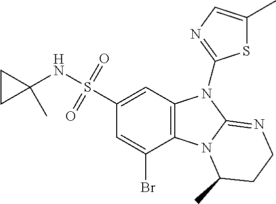 [SMILES: Cc1cnc(N2C3=NCC[C@@H](C)N3c3c(Br)cc(S(=O)(=O)NC4(C)CC4)cc32)s1]

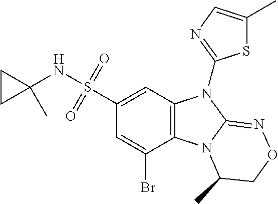 [SMILES: Cc1cnc(N2C3=NOC[C@@H](C)N3c3c(Br)cc(S(=O)(=O)NC4(C)CC4)cc32)s1]

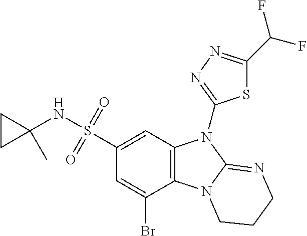 [SMILES: CC1(NS(=O)(=O)c2cc(Br)c3c(c2)N(c2nnc(C(F)F)s2)C2=NCCCN23)CC1]

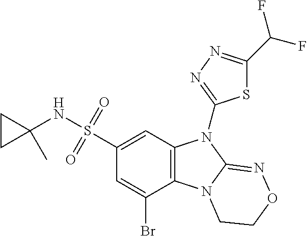 [SMILES: CC1(NS(=O)(=O)c2cc(Br)c3c(c2)N(c2nnc(C(F)F)s2)C2=NOCCN23)CC1]

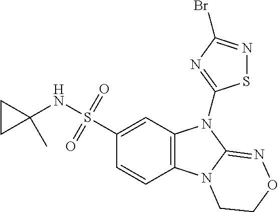 [SMILES: CC1(NS(=O)(=O)c2ccc3c(c2)N(c2nc(Br)ns2)C2=NOCCN23)CC1]

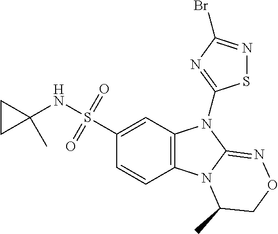 [SMILES: C[C@@H]1CON=C2N(c3nc(Br)ns3)c3cc(S(=O)(=O)NC4(C)CC4)ccc3N21]

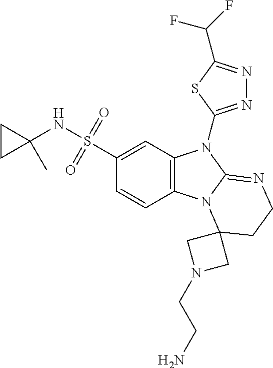 [SMILES: CC1(NS(=O)(=O)c2ccc3c(c2)N(c2nnc(C(F)F)s2)C2=NCCC4(CN(CCN)C4)N23)CC1]

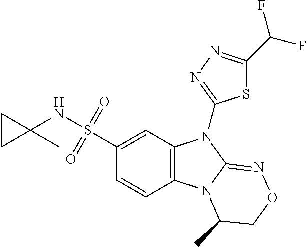 [SMILES: C[C@@H]1CON=C2N(c3nnc(C(F)F)s3)c3cc(S(=O)(=O)NC4(C)CC4)ccc3N21]

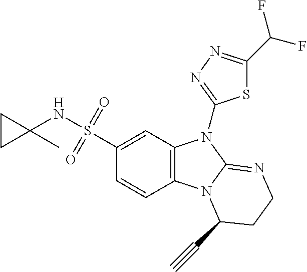 [SMILES: C#C[C@@H]1CCN=C2N(c3nnc(C(F)F)s3)c3cc(S(=O)(=O)NC4(C)CC4)ccc3N21]

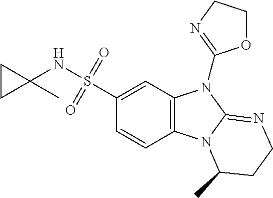 [SMILES: C[C@@H]1CCN=C2N(C3=NCCO3)c3cc(S(=O)(=O)NC4(C)CC4)ccc3N21]

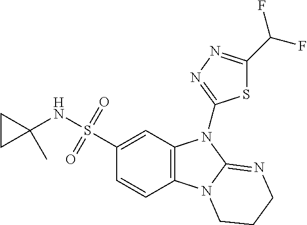 [SMILES: CC1(NS(=O)(=O)c2ccc3c(c2)N(c2nnc(C(F)F)s2)C2=NCCCN23)CC1]

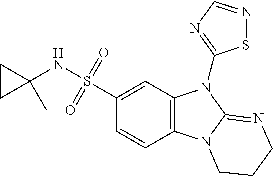 [SMILES: CC1(NS(=O)(=O)c2ccc3c(c2)N(c2ncns2)C2=NCCCN23)CC1]

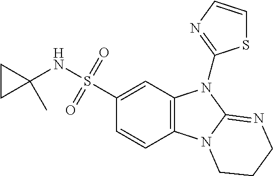 [SMILES: CC1(NS(=O)(=O)c2ccc3c(c2)N(c2nccs2)C2=NCCCN23)CC1]

### Preparation of Compounds

The compounds used in the synthetic chemistry reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester,NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R.V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002. General Synthetic Schemes

The PARG inhibitory compound disclosed herein can be prepared by a variety of synthetic routes including, but not limited to, the routes described below in Scheme I or II.

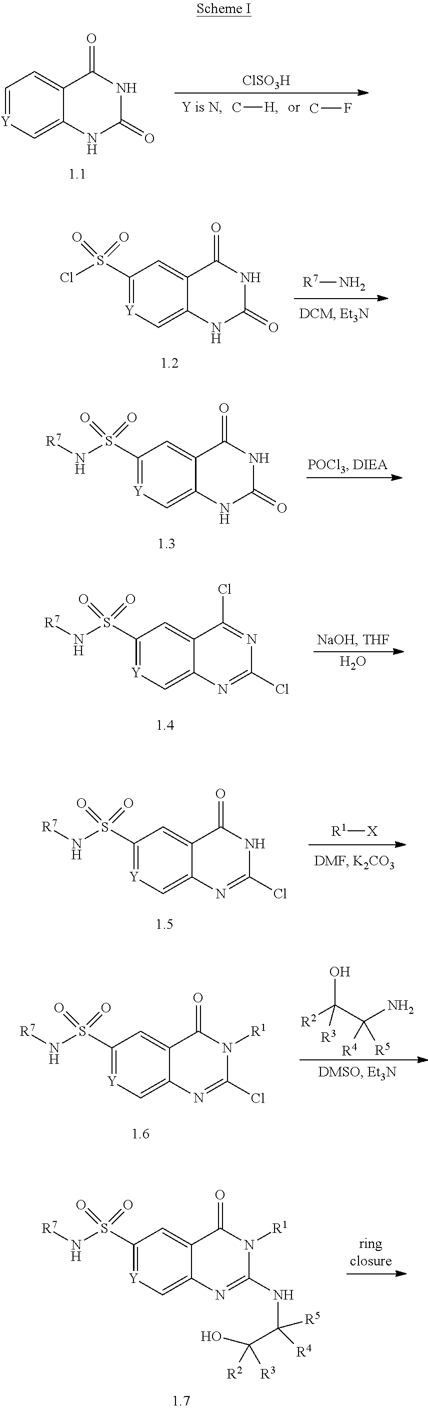

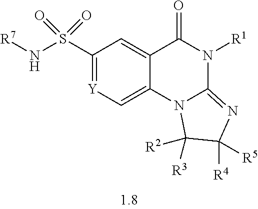

Reaction of 2,4-quinazolinedione derivative 1.1 with chlorosulfonic acid affords the chlorosulfonyl derivative 1.2. Reaction of compound 1.2 with a substituted amine provides sulfonamide 1.3. Chlorination of compound 1.3 affords the dichloroquinazoline derivative 1.4, which undergoes selective hydrolysis at the 4-position upon treatment with aqueous base. 2-chloroquinazolinone 1.5 is substituted on the amide nitrogen by treatment with a suitable electrophile under basic conditions to afford compound 1.6. Annulation of the 5-membered imidazo ring begins with substitution of the chloro group by reaction with a suitable amino alcohol to yield 2-aminoquinazolinone derivative 1.7. Closure of the imidazo ring to obtain target compound 1.8 is accomplished under Mitsunobu conditions (e.g., DEAD, PPh₃), or, alternatively, by activating the alcohol as a suitable leaving group, such as conversion to methanesulfonate or chlorination with SOCl₂, followed by treatment with base to effect ring closure. Persons of skill in the art will recognize that use of a 1,2-amino alcohol will afford the 5-membered imidazo compound 1.8, whereas use of a 1,3-amino alcohol will afford the analogous 6-membered compound. Likewise, use of a 1,4-amino alcohol will afford the analogous 7-membered compound. In the event a single stereoisomer of the target compound is desired, chiral chromatography with supercritical fluid chromatography can be employed. Alternatively, non-racemic, chiral starting materials, such as the amino alcohol may be employed, as appropriate.

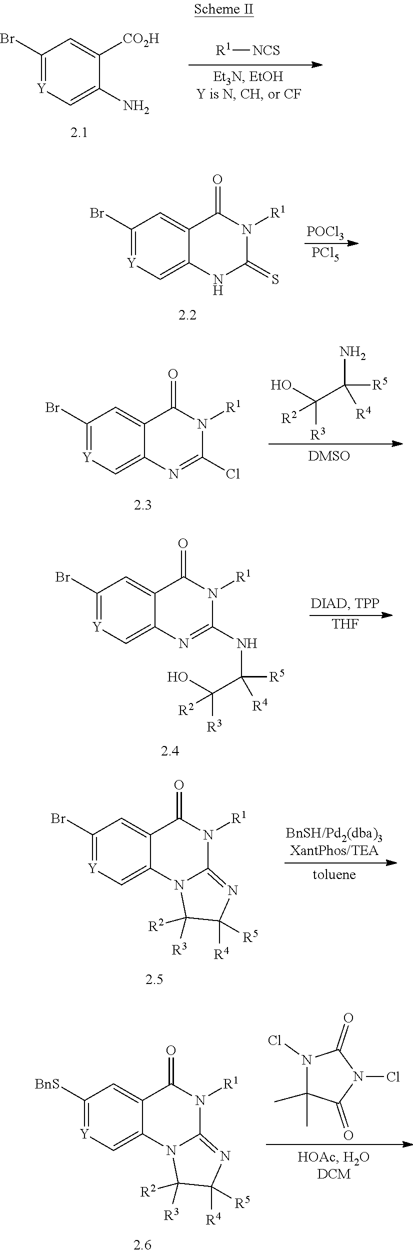

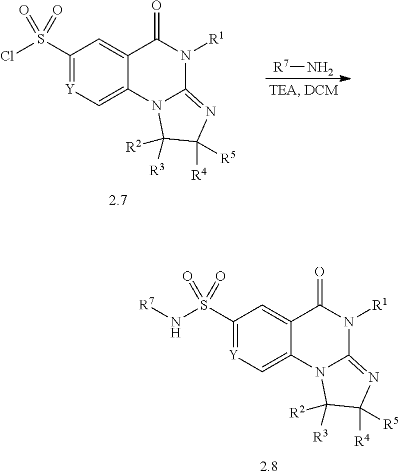

Anthranilic acid derivative 2.1 can be condensed with an alkyl isothiocyanate to afford the 2-thioxo-2,3-dihydroquinazolin-4(1H)-one derivative 2.2. Chlorination provides compound 2.3 which undergoes substitution with an amino alcohol to afford quinazolinone derivative 2.4. Closure of the imidazo ring to obtain compound 2.5 is accomplished under Mitsunobu conditions (e.g., DIAD, PPh₃), or, alternatively, by activating the alcohol as a suitable leaving group, effected by reagents such as conversion to methanesulfonyl chloride (MeSO₂Cl) or thionyl chloride (SOCl₂), followed by treatment with base to effect ring closure. Persons of skill in the art will recognize that use of a 1,2-amino alcohol will afford the 5-membered imidazo compound 2.5, whereas use of a 1,3-amino alcohol will afford the analogous 6-membered compound. Likewise, use of a 1,4-amino alcohol will afford the analogous 7-membered compound. Palladium-catalyzed thiolation of imidazo compound 2.5 affords sulfide 2.6 which undergoes oxidative chlorination to afford sulfonyl chloride 2.7. Reaction of the sulfonyl chloride with an appropriate amine affords target compound 2.8.

Persons of skill in the art of organic synthesis will recognize that variation of Scheme I or II may be necessitated by the nature of the substituents in groups R¹—R⁵ and R⁷. Such modifications may include, for example, the use of protecting groups to alter the reactivity of substituents, or altered time and temperature for reaction. Additionally, further modification of compounds of formula 1.8 or 2.8 may be performed to arrive upon the desired PARG inhibitory compound. In the event a single stereoisomer of the target compound is desired, chiral chromatography with supercritical fluid chromatography can be employed. Alternatively, non-racemic, chiral starting materials, such as the amino alcohol may be employed, as appropriate.

Using appropriate starting materials, the PARG inhibitory compounds described herein by Formula (I)-(III), or within Tables 1 or 2, can be synthesized using the methods described above in Scheme I or II.

### Pharmaceutical Compositions

In certain embodiments, the PARG inhibitory compound described herein is administered as a pure chemical. In other embodiments, the PARG inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one PARG inhibitory compound as described herein, or a stereoisomer, a pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I)-(III), or a pharmaceutically acceptable salt or solvate thereof.

One embodiment provides a method of preparing a pharmaceutical composition comprising mixing a compound of Formula (I)-(III), or a pharmaceutically acceptable salt or solvate thereof, and a pharmaceutically acceptable carrier.

In certain embodiments, the PARG inhibitory compound as described by Formula (I)-(III), or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

One embodiment provides a pharmaceutical composition comprising a pharmaceutically acceptable excipient and a compound of Table 1 or Table 2, or a pharmaceutically acceptable salt or solvate thereof.

One embodiment provides a method of preparing a pharmaceutical composition comprising mixing a compound of Table 1 or Table 2, or a pharmaceutically acceptable salt or solvate thereof, and a pharmaceutically acceptable carrier.

In certain embodiments, the PARG inhibitory compound as described by Table 1 or Table 2, or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the PARG inhibitory compound as described by Formula (I)-(III) or Table 1 or Table 2, or a pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one PARG inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), or a pharmaceutically acceptable salt or solvate thereof, for use in a method of treatment of the human or animal body.

One embodiment provides a compound of Formula (I), or a pharmaceutically acceptable salt or solvate thereof, for use in a method of treatment of cancer or neoplastic disease.

One embodiment provides a pharmaceutical composition comprising a compound of Formula (I), or a pharmaceutically acceptable salt or solvate thereof, and a pharmaceutically acceptable excipient for use in a method of treatment of cancer or neoplastic disease.

One embodiment provides a use of a compound of Formula (I), or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Formula (I), or a pharmaceutically acceptable salt or solvate thereof. In some embodiments is provided a method of treating cancer, in a patient in need thereof, comprising administering to the patient a pharmaceutical composition comprising a compound of Formula (I), or a pharmaceutically acceptable salt or solvate thereof, and a pharmaceutically acceptable excipient.

One embodiment provides a compound of Formula (Ia), or a pharmaceutically acceptable salt or solvate thereof, for use in a method of treatment of the human or animal body.

One embodiment provides a compound of Formula (Ia), or a pharmaceutically acceptable salt or solvate thereof, for use in a method of treatment of cancer or neoplastic disease.

One embodiment provides a pharmaceutical composition comprising a compound of Formula (Ia), or a pharmaceutically acceptable salt or solvate thereof, and a pharmaceutically acceptable excipient for use in a method of treatment of cancer or neoplastic disease.

One embodiment provides a use of a compound of Formula (Ia), or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Formula (Ia), or a pharmaceutically acceptable salt or solvate thereof. In some embodiments is provided a method of treating cancer, in a patient in need thereof, comprising administering to the patient a pharmaceutical composition comprising a compound of Formula (Ia), or a pharmaceutically acceptable salt or solvate thereof, and a pharmaceutically acceptable excipient.

One embodiment provides a compound of Formula (II), or a pharmaceutically acceptable salt or solvate thereof, for use in a method of treatment of the human or animal body.

One embodiment provides a compound of Formula (II), or a pharmaceutically acceptable salt or solvate thereof, for use in a method of treatment of cancer or neoplastic disease.

One embodiment provides a pharmaceutical composition comprising a compound of Formula (II), or a pharmaceutically acceptable salt or solvate thereof, and a pharmaceutically acceptable excipient for use in a method of treatment of cancer or neoplastic disease.

One embodiment provides a use of a compound of Formula (II), or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Formula (II), or a pharmaceutically acceptable salt or solvate thereof. In some embodiments is provided a method of treating cancer, in a patient in need thereof, comprising administering to the patient a pharmaceutical composition comprising a compound of Formula (II), or a pharmaceutically acceptable salt or solvate thereof, and a pharmaceutically acceptable excipient.

One embodiment provides a compound of Formula (III), or a pharmaceutically acceptable salt or solvate thereof, for use in a method of treatment of the human or animal body.

One embodiment provides a compound of Formula (III), or a pharmaceutically acceptable salt or solvate thereof, for use in a method of treatment of cancer or neoplastic disease.

One embodiment provides a pharmaceutical composition comprising a compound of Formula (III), or a pharmaceutically acceptable salt or solvate thereof, and a pharmaceutically acceptable excipient for use in a method of treatment of cancer or neoplastic disease.

One embodiment provides a use of a compound of Formula (III), or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Formula (III), or a pharmaceutically acceptable salt or solvate thereof. In some embodiments is provided a method of treating cancer, in a patient in need thereof, comprising administering to the patient a pharmaceutical composition comprising a compound of Formula (III), or a pharmaceutically acceptable salt or solvate thereof, and a pharmaceutically acceptable excipient.

One embodiment provides a compound of Table 1 or Table 2, or a pharmaceutically acceptable salt or solvate thereof, for use in a method of treatment of the human or animal body.

One embodiment provides a compound of Table 1 or Table 2, or a pharmaceutically acceptable salt or solvate thereof, for use in a method of treatment of cancer or neoplastic disease.

One embodiment provides a pharmaceutical composition comprising a compound of Table 1 or Table 2, or a pharmaceutically acceptable salt or solvate thereof, and a pharmaceutically acceptable excipient for use in a method of treatment of cancer or neoplastic disease.

One embodiment provides a use of a compound of Table 1 or Table 2, or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Table 1 or Table 2, or a pharmaceutically acceptable salt or solvate thereof. In some embodiments is provided a method of treating cancer, in a patient in need thereof, comprising administering to the patient a pharmaceutical composition comprising a compound of Table 1 or Table 2, or a pharmaceutically acceptable salt or solvate thereof, and a pharmaceutically acceptable excipient.

Provided herein is the method wherein the pharmaceutical composition is administered orally. Provided herein is the method wherein the pharmaceutical composition is administered by injection.

One embodiment provides a method of inhibiting a PARG enzyme comprising contacting the PARG enzyme with a compound of Formula (I)-(III) or Table 1 or Table 2.

Another embodiment provides the method of inhibiting a PARG enzyme, wherein the PARG enzyme is contacted in an in vivo setting. Another embodiment provides the method of inhibiting a PARG enzyme, wherein the PARG enzyme is contacted in an in vitro setting.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the PARG inhibitory compounds disclosed herein are synthesized according to the following examples. As used below, and throughout the description of the invention, the following abbreviations, unless otherwise indicated, shall be understood to have the following meanings:

| | |
|---|---|
| ACN | acetonitrile |
| ° C. | degrees Celsius |
| δ_{H} | chemical shift in parts per million |
| | downfield from tetramethylsilane |
| DCM | dichloromethane (CH₂Cl₂) |
| DIAD | diisopropyl azodicarboxylate |
| DIEA | diisopropylethylamine |
| DMF | dimethylformamide |
| DMSO | dimethylsulfoxide |
| EA | ethyl acetate |
| EtOAc | ethyl acetate |
| ESI | electrospray ionization |
| Et | ethyl |
| g | gram(s) |
| h | hour(s) |
| HPLC | high performance liquid chromatography |
| Hz | hertz |
| J | coupling constant (in NMR spectrometry) |
| LCMS | liquid chromatography mass spectrometry |
| μ | micro |
| m | multiplet (spectral); meter(s); milli |
| M | molar |
| M⁺ | parent molecular ion |
| Me | methyl |
| MsCl | methanesulfonyl chloride |
| MHz | megahertz |
| min | minute(s) |
| mol | mole(s); molecular (as in mol wt) |
| mL | milliliter |
| MS | mass spectrometry |
| nm | nanometer(s) |
| NMR | nuclear magnetic resonance |
| pH | potential of hydrogen; a measure of the |
| | acidity or basicity of an aqueous solution |
| PE | petroleum ether |
| RT | room temperature |
| s | singlet (spectral) |
| t | triplet (spectral) |
| SFC | Supercritical fluid chromatography |
| T | temperature |
| TFA | trifluoroacetic acid |
| THF | tetrahydrofuran |
| TPP | Triphenylphosphine |

Representative Synthesis Route 1: Example 1: (R)-9-cyclopropyl-1-methyl-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

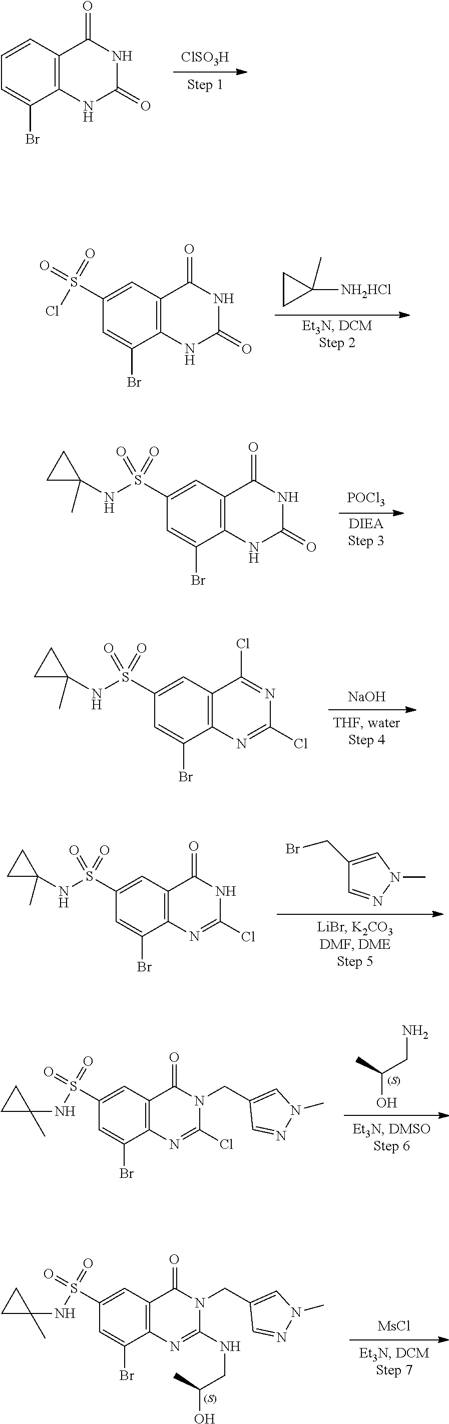

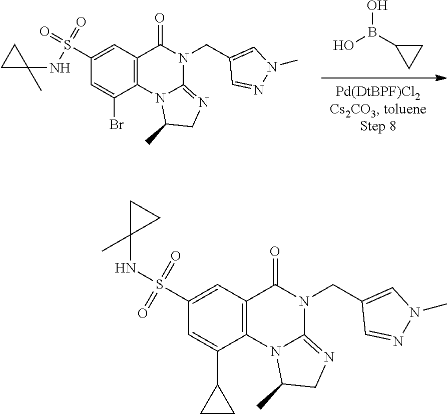

Step 1: A solution of 8-bromo-1,3-dihydroquinazoline-2,4-dione (20 g, 82.9 mmol) in chlorosulfonic acid (200 mL) was stirred overnight at 80° C. The reaction was quenched by the addition of ice (1000 g) at 0° C. The precipitated solids were collected by filtration and washed with water (3×100 mL). The resulting solid was dried under vacuum to obtain 8-bromo-2,4-dioxo-1,3-dihydroquinazoline-6-sulfonyl chloride (30 g) as a white solid. The crude product was used in the next step directly without further purification. LCMS (ESI) m/z: 339, 341 [M+H]⁺.

Step 2: A solution of 8-bromo-2,4-dioxo-1,3-dihy droquinazoline-6-sulfonyl chloride (30 g, 88.3 mmol), 1-methylcyclopropan-1-aminehydrochloride (11.4 g, 106 mmol) and TEA (26.8 g, 265 mmol) in DCM (500 mL) was stirred for 2 h at room temperature. The resulting mixture was concentrated under reduced pressure. The residue was purified by silica gel column chromatography, eluted with CH₂Cl₂/EA (1:1) to afford 8-bromo-N-(1-methylcyclopropyl)-2,4-dioxo-1,3-dihydroquinazoline-6-sulfonamide(25 g, 76%). LCMS (ESI) m/z: 374, 376 [M+H]⁺.

Step 3: A mixture of 8-bromo-N-(1-methylcyclopropyl)-2,4-dioxo-1,3-dihydroquinazoline-6-sul fonamide (5 g, 13.4 mmol) and DIEA (3.4 g, 26.7 mmol) in POCl₃ (100 mL) was stirred at 105° C. overnight. The resulting mixture was concentrated under reduced pressure. The reaction was q uenched by the addition of water/ice (150 mL) at room temperature. The resulting mixture was e xtracted with EtOAc (3×200 mL). The combined organic layers were washed with brine (2×5 0 mL), dried over anhydrous Na₂SO₄. After filtration, the filtrate was concentrated under reduce d pressure to afford 8-bromo-2,4-dichloro-N-(1-methylcyclopropyl)quinazoline-6-sulfonamide (5 g, crude) as a brown solid, which was used in the next step directly without further purification.

Step 4: To a stirred mixture of 8-bromo-2,4-dichloro-N-(1-methylcyclopropyl)quinazoline-6-sul fonamide (2 g, 4.86 mmol) in THF (20 mL) and H₂O (20 mL) was added NaOH (0.39 g, 9.73 m mol) at 0° C. The resulting mixture was stirred for 30 min at 0° C. The mixture was acidified to p H 6 with conc. HCl. The reaction was quenched by the addition of water (200 mL) at 0° C. The resulting mixture was extracted with EtOAc (3×250 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (45%-55%) to afford 8-bromo-2-chloro-N-(1-methylcyclopropyl)-4-oxo-3 H-quinazoline-6-sulfonamide (500 mg) as a yellow solid. LCMS (ESI) m/z: 392, 394 [M+H]⁺.

Step 5: To a stirred mixture of 8-bromo-2-chloro-N-(1-methylcyclopropyl)-4-oxo-3H-quinazolin e-6-sulfonamide (350 mg, 0.89 mmol) and LiBr (77.4 mg, 0.89 mmol) in DMF (8 mL) and DME (2 mL) were added K₂CO₃ (369.6 mg, 2.67 mmol) in portions at room temperature. The resulting mixture was stirred for 15 min at 0° C. To the above mixture was added 4-(bromomethyl)-1-methylpyrazole (312 mg, 1.78 mmol) in portions over 1 min at room temperature. The resulting mixture was stirred overnight at room temperature. The reaction was quenched by the addition o f water (150 mL) at room temperature. The resulting mixture was extracted with EtOAc (3×160 mL). The combined organic layers were washed with brine (2×10 mL), dried over anhydrous N a₂SO₄. After filtration, the filtrate was concentrated under reduced pressure. The residue was purified by silica gel column chromatography, eluted with CH₂Cl₂/MeOH (45%-55%) to afford 8-bromo-2-chloro-N-(1-methylcyclopropyl)-3-[(1-methylpyrazol-4-yl)methyl]-4-oxoquinazoline-6-sulfonamide (350 mg) as a yellow solid. LCMS (ESI) m/z: 486, 488 [M+H]⁺.

Step 6: To a stirred mixture of 8-bromo-2-chloro-N-(1-methylcyclopropyl)-3-[(1-methylpyrazol-4-yl)methyl]-4-oxoquinazoline-6-sulfonamide (300 mg, 0.62 mmol) and (2S)-1-aminopropan-2-o 1(69.4 mg, 0.924 mmol) in DMSO (10 mL) was added TEA (187 mg, 1.85 mmol) in portions at room temperature. The resulting mixture was stirred for 30 min at room temperature. The reaction was quenched by the addition of water (150 mL) at room temperature. The resulting mixture w as extracted with EtOAc (3×160 mL). The combined organic layers were washed with brine (2×10 mL), dried over anhydrous Na₂SO₄. After filtration, the filtrate was concentrated under reduced pressure. This resulted in the desired product (300 mg) as a light yellow solid, which was us ed in the next step directly without further purification. LCMS (ESI) m/z: 525, 527 [M+H]⁺.

Step 7: To product from step 6 (300 mg, 0.57 mmol), MsCl (327 mg, 2.85 mmol) and TEA (289 mg, 2.85 mmol) in DCM (10 mL) was stirred for 16 h at room temperature. The reaction was quenched by the addition of water (10 mL) at room temperature. The resulting mixture was concentrated under reduced pressure. The residue was purified by silica gel column chromatography, eluted with CH₂Cl₂/MeOH (9:1) to afford (1R)-9-bromo-1-methyl-N-(1-methylcyclopropyl)-4-[(1-methylpyrazol-4-yl)methyl]-5-oxo-1H,2H-imidazo[1,2-a]quinazoline-7-sulfonamide (220 mg, 76%) as a yellow solid. LCMS (ESI) m/z: 507, 509 [M+H]⁺.

Step 8: To a stirred mixture of (1R)-9-bromo-1-methyl-N-(1-methylcyclopropyl)-4-[(1-methylpyrazol-4-yl)methyl]-5-oxo-1H, 2H-imidazo[1,2-a]quinazoline-7-sulfonamide (10 mg, 0.020 mmol) and cyclopropylboronic acid (2.5 mg, 0.03 mmol) in toluene (2 mL) were added Cs₂CO₃ (1.6 mg, 0.005 mmol) and Pd(DtBPF)Cl₂ (6.4 mg, 0.01 mmol) in portions at room temperature. The resulting mixture was stirred for 2 h at 90° C. under nitrogen atmosphere. The reaction was quenched by the addition of water (50 mL) at room temperature. The resulting mixture was extracted with EtOAc (3×60 mL). The combined organic layers were washed with brine (2×10 mL), dried over anhydrous Na₂SO₄. After filtration, the filtrate was concentrated underreduced pressure. The residue was purified by silica gel column chromatography, eluted with CH₂Cl₂/MeOH (0%-10%). The crude product was purified by Prep-HPLC with the following conditions (Column: YMC-Actus Triart C18 ExRS 30*150 mm, 5 m; Mobile Phase A: water (10 mmol/L NH₄HCO 3), Mobile Phase B: ACN; Flow rate: 60 mL/min mL/min; Gradient: 26% B to 43% B in 10 min; Wave Length: 254 nm/220 nm; RT1(min): 10.88) to afford (1R)-9-cyclopropyl-1-methyl-N-(1-methylcyclopropyl)-4-[(1-methylpyrazol-4-yl)methyl]-5-oxo-1H,2H-imidazo[1,2-a]quinazoline-7-sulfonamide (6.9 mg, 15%). LCMS (ESI) m/z: 469.19 [M+H]⁺. ¹H NMR (400 MHz, DMSO-d₆) δ 8.34-8.24 (m, 2H), 7.96 (d, J=2.2 Hz, 1H), 7.79 (s, 1H), 7.53 (s, 1H), 5.98 (d, J=7.9 Hz, 1H), 5.18 (d, J=15.5 Hz, 1H), 4.94 (d, J=15.5 Hz, 1H), 4.12 (q, J=11.3, 8.5 Hz, 2H), 3.79 (s, 3H), 2.32-2.29 (m, 1H), 1.34 (d, J=6.6 Hz, 3H), 1.27-1.20 (m, 1H), 1.10-1.05 (m, 4H), 0.9 1-0.89 (m, 1H), 0.77-0.75 (m, 1H), 0.66-0.52 (m, 2H), 0.46-0.34 (m, 2H).

The following compounds in Table 3 were prepared using procedures similar to those described in Representative Synthesis Route 1 for Example 1 using appropriate starting

TABLE 3

| Example | | |
|---|---|---|
| No. | Name | [M + H]⁺ |
| 3 | 9-bromo-2,2-dimethyl-4-((1-methyl-1H-pyrazol-4-yl)methyl)- | 521.00, |
| | N-(1-methylcyclopropyl)-5-oxo-1,2,4,5- | 523.00 |
| | tetrahydroimidazo[1,2-a]quinazoline-7-sulfonamide | |
| 18 | 9-bromo-4-((1-methyl-1H-pyrazol-4-yl)methyl)-N-(1- | 493.00, |
| | methylcyclopropyl)-5-oxo-1,2,4,5-tetrahydroimidazo[1,2- | 495.00 |
| | a]quinazoline-7-sulfonamide | |
| 19 | (S)-1-(isopropoxymethyl)-4-((1-methyl-1H-pyrazol-4- | 487.15 |
| | yl)methyl)-N-(1-methylcyclopropyl)-5-oxo-1,2,4,5- | |
| | tetrahydroimidazo[1,2-a]quinazoline-7-sulfonamide | |
| 23 | (R)-9-bromo-1-methyl-4-((1-methyl-3-(trifluoromethyl)-1H- | 575.00, |
| | pyrazol-4-yl)methyl)-N-(1-methylcyclopropyl)-5-oxo-1,2,4,5- | 577.00 |
| | tetrahydroimidazo[1,2-a]quinazoline-7-sulfonamide | |
| 24 | (R)-4-((3-(difluoromethyl)-1-methyl-1H-pyrazol-4- | 479.05 |
| | yl)methyl)-1-methyl-N-(1-methylcyclopropyl)-5-oxo-1,2,4,5- | |
| | tetrahydroimidazo[1,2-a]quinazoline-7-sulfonamide | |
| 28 | (S)-4-((1-methyl-1H-pyrazol-4-yl)methyl)-N-(1- | 521.15 |
| | methylcyclopropyl)-5-oxo-1-(phenoxymethyl)-1,2,4,5- | |
| | tetrahydroimidazo[1,2-a]quinazoline-7-sulfonamide | |
| 29 | (S)-1-((4-fluorophenoxy)methyl)-4-((1-methyl-1H-pyrazol-4- | 539.05 |
| | yl)methyl)-N-(1-methylcyclopropyl)-5-oxo-1,2,4,5- | |
| | tetrahydroimidazo[1,2-a]quinazoline-7-sulfonamide | |
| 30 | (S)-1-((3-fluorophenoxy)methyl)-4-((1-methyl-1H-pyrazol-4- | 539.10 |
| | yl)methyl)-N-(1-methylcyclopropyl)-5-oxo-1,2,4,5- | |
| | tetrahydroimidazo[1,2-a]quinazoline-7-sulfonamide | |
| 31 | (R)-1-(3-(dimethylamino)prop-1-yn-1-yl)-4-((1-methyl-1H- | 496.05 |
| | pyrazol-4-yl)methyl)-N-(1-methylcyclopropyl)-5-oxo-1,2,4,5- | |
| | tetrahydroimidazo[1,2-a]quinazoline-7-sulfonamide | |
| 34 | (R)-9-bromo-1-(cyclopropylethynyl)-4-methyl-N-(1- | 477.05, |
| | methylcyclopropyl)-5-oxo-1,2,4,5-tetrahydroimidazo[1,2- | 478.95 |
| | a]quinazoline-7-sulfonamide | |
| 36 | (R)-4-((1-methyl-1H-pyrazol-4-yl)methyl)-N-(1- | 515.15 |
| | methylcyclopropyl)-5-oxo-1-(phenylethynyl)-1,2,4,5- | |
| | tetrahydroimidazo[1,2-a]quinazoline-7-sulfonamide | |
| 38 | (R)-9-bromo-1-ethynyl-4-methyl-N-(1-methylcyclopropyl)-5- | 436.85, |
| | oxo-1,2,4,5-tetrahydroimidazo[1,2-a]quinazoline-7- | 438.80 |
| | sulfonamide | |
| 39 | (R)-4-((1-methyl-1H-pyrazol-4-yl)methyl)-N-(1- | 497.00 |
| | methylcyclopropyl)-5-oxo-1-(2,2,2-trifluoroethyl)-1,2,4,5- | |
| | tetrahydroimidazo[1,2-a]quinazoline-7-sulfonamide | |
| 40 | (R,E)-4-((1-methyl-1H-pyrazol-4-yl)methyl)-N-(1- | 517.20 |
| | methylcyclopropyl)-5-oxo-1-styryl-1,2,4,5- | |
| | tetrahydroimidazo[1,2-a]quinazoline-7-sulfonamide | |
| 42 | (R)-9-bromo-4-methyl-N-(1-methylcyclopropyl)-5-oxo-1- | 451.00, |
| | (prop-1-yn-1-yl)-1,2,4,5-tetrahydroimidazo[1,2-a]quinazoline- | 452.95 |
| | 7-sulfonamide | |
| 44 | (R)-4-((1-methyl-1H-pyrazol-4-yl)methyl)-N-(1- | 499.05 |
| | methylcyclopropyl)-5-oxo-1-(tetrahydro-2H-pyran-4-yl)- | |
| | 1,2,4,5-tetrahydroimidazo[1,2-a]quinazoline-7-sulfonamide | |
| 45 | (R)-1-(4,4-difluorocyclohexyl)-4-((1-methyl-1H-pyrazol-4- | 533.15 |
| | yl)methyl)-N-(1-methylcyclopropyl)-5-oxo-1,2,4,5- | |
| | tetrahydroimidazo[1,2-a]quinazoline-7-sulfonamide | |
| 47 | (S)-1-(difluoromethyl)-4-((1-methyl-1H-pyrazol-4-yl)methyl)- | 464.95 |
| | N-(1-methylcyclopropyl)-5-oxo-1,2,4,5- | |
| | tetrahydroimidazo[1,2-a]quinazoline-7-sulfonamide | |
| 48 | (R)-1-cyclopentyl-4-((1-methyl-1H-pyrazol-4-yl)methyl)-N- | 483.20 |
| | (1-methylcyclopropyl)-5-oxo-1,2,4,5-tetrahydroimidazo[1,2- | |
| | a]quinazoline-7-sulfonamide | |
| 51 | (R)-9-chloro-1-ethynyl-4-methyl-N-(1-methylcyclopropyl)-5- | 393.05 |
| | oxo-1,2,4,5-tetrahydroimidazo[1,2-a]quinazoline-7- | |
| | sulfonamide | |
| 56 | (R)-9-chloro-1-(3-(dimethylamino)prop-1-yn-1-yl)-4-methyl- | 449.95 |
| | N-(1-methylcyclopropyl)-5-oxo-1,2,4,5- | |
| | tetrahydroimidazo[1,2-a]quinazoline-7-sulfonamide | |
| 64 | (R)-4-(3-hydroxypropyl)-1-methyl-N-(1-methylcyclopropyl)- | 393.00 |
| | 5-oxo-1,2,4,5-tetrahydroimidazo[1,2-a]quinazoline-7- | |
| | sulfonamide | |
| 69 | (R)-1-(3-hydroxy-3-methylbut-1-yn-1-yl)-4-((1-methyl-1H- | 497.15 |
| | pyrazol-4-yl)methyl)-N-(1-methylcyclopropyl)-5-oxo-1,2,4,5- | |
| | tetrahydroimidazo[1,2-a]quinazoline-7-sulfonamide | |
| 70 | (R)-4-((1-methyl-1H-pyrazol-4-yl)methyl)-N-(1- | 512.15 |
| | methylcyclopropyl)-1-(1-methylpiperidin-4-yl)-5-oxo-1,2,4,5- | |
| | tetrahydroimidazo[1,2-a]quinazoline-7-sulfonamide | |
| 72 | (R)-9-bromo-1-ethynyl-4-((1-methyl-1H-pyrazol-4- | 517.05, |
| | yl)methyl)-N-(1-methylcyclopropyl)-5-oxo-1,2,4,5- | 518.95 |
| | tetrahydroimidazo[1,2-a]quinazoline-7-sulfonamide | |
| 81 | (R)-1-(3-(diethylamino)prop-1-yn-1-yl)-4-((1-methyl-1H- | 524.20 |
| | pyrazol-4-yl)methyl)-N-(1-methylcyclopropyl)-5-oxo-1,2,4,5- | |
| | tetrahydroimidazo[1,2-a]quinazoline-7-sulfonamide | |
| 87 | (R)-4-((1-methyl-1H-pyrazol-4-yl)methyl)-N-(1- | 522.20 |
| | methylcyclopropyl)-5-oxo-1-(3-(pyrrolidin-1-yl)prop-1-yn-1- | |
| | yl)-1,2,4,5-tetrahydroimidazo[1,2-a]quinazoline-7- | |
| | sulfonamide | |
| 89 | (R)-1-(azetidin-3-ylethynyl)-4-((1-methyl-1H-pyrazol-4- | 494.10 |
| | yl)methyl)-N-(1-methylcyclopropyl)-5-oxo-1,2,4,5- | |
| | tetrahydroimidazo[1,2-a]quinazoline-7-sulfonamide | |
| 93 | (S)-1-cyclopentyl-4-((1-methyl-1H-pyrazol-4-yl)methyl)-N- | 483.20 |
| | (1-methylcyclopropyl)-5-oxo-1,2,4,5-tetrahydroimidazo[1,2- | |
| | a]quinazoline-7-sulfonamide | |
| 94 | (S)-9-bromo-1-ethynyl-4-((1-methyl-1H-pyrazol-4- | 517.05, |
| | yl)methyl)-N-(1-methylcyclopropyl)-5-oxo-1,2,4,5- | 518.95 |
| | tetrahydroimidazo[1,2-a]quinazoline-7-sulfonamide | |
| 100 | (R)-9-chloro-1-(3-(dimethylamino)prop-1-yn-1-yl)-4-((1- | 530.17 |
| | methyl-1H-pyrazol-4-yl)methyl)-N-(1-methylcyclopropyl)-5- | |
| | oxo-1,2,4,5-tetrahydroimidazo[1,2-a]quinazoline-7- | |
| | sulfonamide | |

Representative Synthesis Route 2: Example 2: (R)-9-(azetidin-1-yl)-1-methyl-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

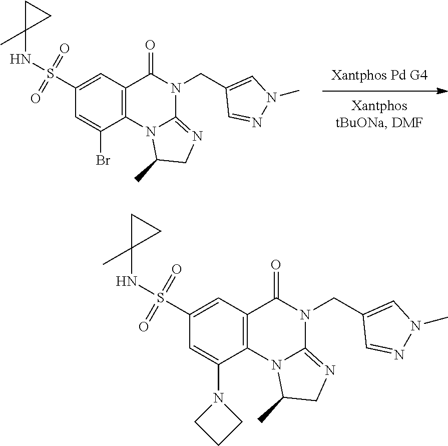

To a stirred solution of (1R)-9-bromo-1-methyl-N-(1-methylcyclopropyl)-4-[(1-methylpyrazol-4-yl)methyl]-5-oxo-1H,2H-imidazo[1,2-a]quinazoline-7-sulfonamide (20 mg, 0.039 mmol) in anhydrous DMF (5 mL) was added azetidine (4.5 mg, 0.078 mmol) and t-BuONa (7.6 mg, 0.078 mmol) followed by catalytic amount of Xantphos Pd G4 (3.8 mg, 0.004 mmol) and XantPhos (2.28 mg, 0.004 mmol) at room temperature. The reaction mixture was stirred at 120° C. overnight. The reaction mixture was purified by Prep-HPLC with the following conditions (Column: Sunfire prep C18 column, 30×150 mm, 5m; Mobile Phase A: water (0.1% FA), Mobile Phase B: ACN; Flow rate: 60 mL/min mL/min; Gradient: 2% B to 22% B in 10 min; Wave Length: 254 nm/220 nm; RT1(min): 12.37) to afford (1R)-9-(azetidin-1-yl)-1-methyl-N-(1-methylcyclopropyl)-4-[(1-methylpyrazol-4-yl)methyl]-5-oxo-1H,2H-imidazo[1,2-a]quinazoline-7-sulfonamide (2.0 mg, 11%). LCMS (ESI) m/z: 484.05 [M+H]⁺. ¹H NMR (400 MHz, DMSO-d₆) δ 8.01 (s, 1H), 7.91 (d, J=2.0 Hz, 1H), 7.68 (s, 1H), 7.41 (s, 2H), 5.02 (t, J=6.9 Hz, 1H), 4.93 (d, J=5.9 Hz, 2H), 3.96 (q, J=7.0 Hz, 2H), 3.87 (dd, J=13.4, 7.8 Hz, 1H), 3.76 (s, 3H), 3.46-3.40 (m, 3H), 2.25 (p, J=7.0 Hz, 2H), 1.04 (s, 3H), 0.90 (d, J=6.1 Hz, 3H), 0.62-0.60 (m, 2H), 0.41-0.33 (m, 2H).

Representative Synthesis Route 3: Example 11: 9-(cyclopent-1-en-1-yl)-2,2-dimethyl-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

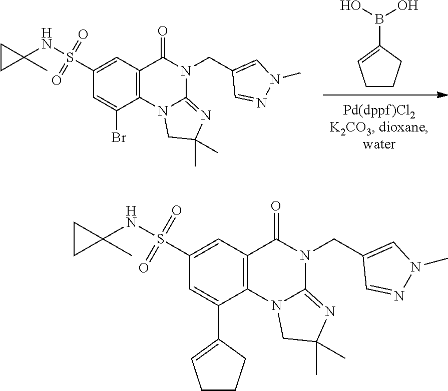 [SMILES: Cn1cc(CN2C(=O)c3cc(S(=O)(=O)NC4(C)CC4)cc(Br)c3N3CC(C)(C)N=C23)cn1 ; OB(O)C1=CCCC1 ; "Pd(dppf)Cl2K2CO3, dioxane,water" ; Cn1cc(CN2C(=O)c3cc(S(=O)(=O)NC4(C)CC4)cc(C4=CCCC4)c3N3CC(C)(C)N=C23)cn1]

To a microwave vial added 9-bromo-2,2-dimethyl-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide (11 mg, 0.021 mmol), K₂CO₃ (11.7 mg, 0.084 mmol), (1-cyclopenten-1-yl)boranediol (3.1 mg, 0.027 mmol), dioxane (1.6 mL), water (0.5 mL) degassed and back filled with N₂ there times.

Subsequently added Pd(dppf)Cl₂ (1.48 mg, 0.002 mmol) degassed and back filled with N₂ and heated in a microwave at 135° C. for 1h. Crude reaction mixture was purified by silica gel chromatography MeOH/DCM (0-20%) to obtain 9-(cyclopent-1-en-1-yl)-2,2-dimethyl-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide (6 mg, 560). LCMS (ESI) m/z: 509.00 [M+H]⁺. ¹H NMR (400 MHz, CDCl₃) δ 8.44 (d, J=2.4 Hz, 111), 7.70 (d, J=2.4 Hz, 111), 7.66 (s, 111), 7.56 (s, 111), 5.73 (s, 111), 5.05 (s, 211), 4.92 (s, 111), 3.84 (s, 311), 3.70-3.73 (in, 111), 2.57-2.54 (in, 411), 2.07-2.05 (in, 211), 1.35 (s, 611), 1.23 (s, 311), 0.78-0.76 (m 211), 0.49-0.47 (in, 211).

The following compounds in Table 4 were prepared using procedures similar to those described in Representative Synthesis Route 3 for Example 11 using appropriate starting

TABLE 4

| Example | | |
|---|---|---|
| No. | Name | [M + H]⁺ |
| 4 | 2,2-dimethyl-9-(1-methyl-1,2,3,6-tetrahydropyridin-4-yl)-4- | 538.00 |
| | ((1-methyl-1H-pyrazol-4-yl)methyl)-N-(1- | |
| | methylcyclopropyl)-5-oxo-1,2,4,5-tetrahydroimidazo[1,2- | |
| | a]quinazoline-7-sulfonamide | |
| 12 | 9-(3,6-dihydro-2H-pyran-4-yl)-2,2-dimethyl-4-((1-methyl-1H- | 525.00 |
| | pyrazol-4-yl)methyl)-N-(1-methylcyclopropyl)-5-oxo-1,2,4,5- | |
| | tetrahydroimidazo[1,2-a]quinazoline-7-sulfonamide | |
| 17 | 9-(4-fluorophenyl)-2,2-dimethyl-4-((1-methyl-1H-pyrazol-4- | 537.01 |
| | yl)methyl)-N-(1-methylcyclopropyl)-5-oxo-1,2,4,5- | |
| | tetrahydroimidazo[1,2-a]quinazoline-7-sulfonamide | |
| 20 | 9-(4-fluoro-2-methylphenyl)-4-((1-methyl-1H-pyrazol-4- | 523.00 |
| | yl)methyl)-N-(1-methylcyclopropyl)-5-oxo-1,2,4,5- | |
| | tetrahydroimidazo[1,2-a]quinazoline-7-sulfonamide | |
| 21 | 9-(4-chlorophenyl)-4-((1-methyl-1H-pyrazol-4-yl)methyl)-N- | 525.00 |
| | (1-methylcyclopropyl)-5-oxo-1,2,4,5-tetrahydroimidazo[1,2- | |
| | a]quinazoline-7-sulfonamide | |
| 25 | 9-(4-fluoro-2-methylphenyl)-2,2-dimethyl-4-((1-methyl-1H- | 551.00 |
| | pyrazol-4-yl)methyl)-N-(1-methylcyclopropyl)-5-oxo-1,2,4,5- | |
| | tetrahydroimidazo[1,2-a]quinazoline-7-sulfonamide | |
| 26 | 9-(cyclopent-1-en-1-yl)-4-((1-methyl-1H-pyrazol-4- | 481.00 |
| | yl)methyl)-N-(1-methylcyclopropyl)-5-oxo-1,2,4,5- | |
| | tetrahydroimidazo[1,2-a]quinazoline-7-sulfonamide | |

The following compounds in Table 5 were prepared using procedures similar to those described in Representative Synthesis Route 3 for Example 11, but using conventional heating at 100° C. for 10 min, using appropriate starting materials.

TABLE 5

| Example | | |
|---|---|---|
| No. | Name | [M − H] |
| 58 | N-methyl-5-(2,2,4-trimethyl-7-(N-(1- | 495.00 |
| | methylcyclopropyl)sulfamoyl)-5-oxo-1,2,4,5- | |
| | tetrahydroimidazo[1,2-a]quinazolin-9-yl)picolinamide | |
| 59 | 9-(1-(cyclopropanecarbonyl)-1,2,3,6-tetrahydropyridin-4-yl)- | 510.00 |
| | 2,2,4-trimethyl-N-(1-methylcyclopropyl)-5-oxo-1,2,4,5- | |
| | tetrahydroimidazo[1,2-a]quinazoline-7-sulfonamide | |
| 60 | 9-(6-methoxypyridin-3-yl)-2,2,4-trimethyl-N-(1- | 468.00 |
| | methylcyclopropyl)-5-oxo-1,2,4,5-tetrahydroimidazo[1,2- | |
| | a]quinazoline-7-sulfonamide | |
| 61 | 9-(4-fluoro-2-methylphenyl)-2,2,4-trimethyl-N-(1- | 469.00 |
| | methylcyclopropyl)-5-oxo-1,2,4,5-tetrahydroimidazo[1,2- | |
| | a]quinazoline-7-sulfonamide | |
| 62 | (R)-9-(2-(hydroxymethyl)phenyl)-1,4-dimethyl-N-(1- | 453.00 |
| | methylcyclopropyl)-5-oxo-1,2,4,5-tetrahydroimidazo[1,2- | |
| | a]quinazoline-7-sulfonamide | |
| 63 | (R)-9-(2-cyanophenyl)-1,4-dimethyl-N-(1- | 448.00 |
| | methylcyclopropyl)-5-oxo-1,2,4,5-tetrahydroimidazo[1,2- | |
| | a]quinazoline-7-sulfonamide | |

Representative Synthesis Route 4: Example 5: (R)-4-((3-ethyl-5-methylisoxazol-4-yl)methyl)-1-methyl-N-(1-methylcyclopropyl)-5-oxo-1,2,4,5-tetrahydroimidazo[1,2-a]quinazoline-7-sulfonamide

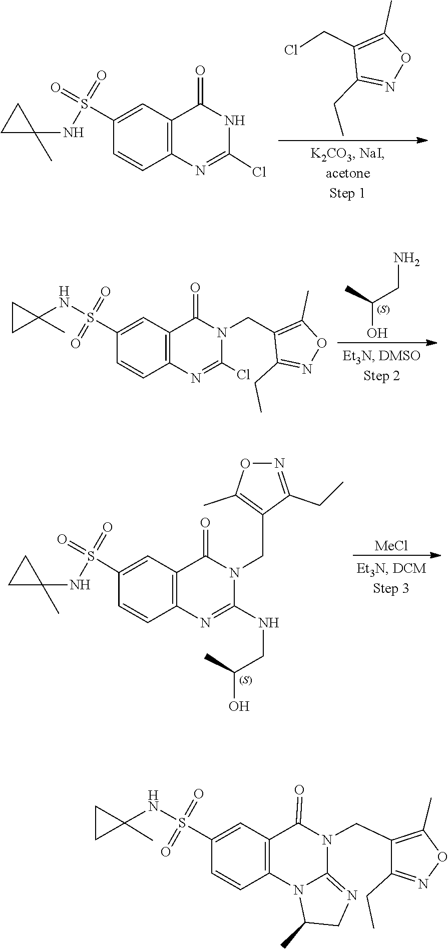

Step 1: To a stirred mixture of 2-chloro-N-(1-methylcyclopropyl)-4-oxo-3H-quinazoline-6-sulfonamide (82 mg, 0.26 mmol) in acetone (1.6 mL) were added 4-(chloromethyl)-3-ethyl-5-methylisoxazole(41.7 mg, 0.26 mmol), NaI (3.9 mg, 0.026 mmol) and K₂CO₃ (72.2 mg, 0.52 mmol) at room temperature. The resulting mixture was stirred at room temperature overnight.

The reaction mixture was washed with water and brine. The resulting crude mixture was purified by silica gel column chromatography, eluted with EtOAc/hexane (0-100%) to afford 2-chloro-3-[(3-ethyl-5-methyl-4-isoxazolyl)methyl]-6-(1-methylcyclopropylaminosulfonyl)-3,4-dihydro-4-quinazolinone (65 mg, 57%) as a yellow solid. LCMS (ESI) m/z: 437 [M+H]⁺.

Synthesis of Example 5 was completed (steps 2 and 3) using procedures similar to those described in Representative Synthesis Route 1 for Example 1, steps 6 and 7 using appropriate starting materials. LCMS (ESI) m/z: 458.00 [M+H]⁺. 1H NMR (400 MHz, DMSO-d₆) δ 8.23 (d, J=2.0 Hz, 1H), 8.03 (s, 1H), 7.92-7.90 (m, 1H), 7.26-7.24 (m, 1H), 4.94-4.84 (m, 2H), 4.74-4.70 (m, 1H), 4.04-4.00 (m, 1H), 3.52-3.49 (m, 1H), 2.72-2.67 (m, 2H), 2.43 (s, 3H), 1.31-1.29 (m, 3H), 1.12-1.09 (m, 3H), 1.06 (s, 3H), 0.60-0.58 (m, 2H), 0.38-0.36 (m, 2H).

The following compounds in Table 6 were prepared using procedures similar to those described in Representative Synthesis Route 4 for Example 5 using appropriate starting materials.

TABLE 6

| Example | | |
|---|---|---|
| No. | Name | [M + H]⁺ |
| 7 | (R)-4-((3-(methoxymethyl)isoxazol-4-yl)methyl)-1-methyl-N- | 460.00 |
| | (1-methylcyclopropyl)-5-oxo-1,2,4,5-tetrahydroimidazo[1,2- | |
| | a]quinazoline-7-sulfonamide | |
| 9 | (R)-4-(4-methoxybenzyl)-1-methyl-N-(1-methylcyclopropyl)- | 455.00 |
| | 5-oxo-1,2,4,5-tetrahydroimidazo[1,2-a]quinazoline-7- | |
| | sulfonamide | |

Representative Synthesis Route 5: Example 6: (R)-9-(5-fluoropyridin-2-yl)-1-methyl-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

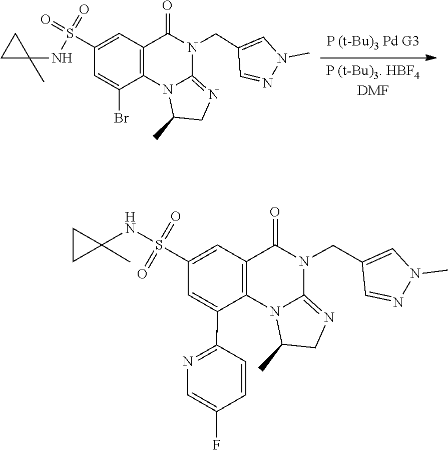 [SMILES: C[C@@H]1CN=C2N(Cc3cnn(C)c3)C(=O)c3cc(S(=O)(=O)NC4(C)CC4)cc(Br)c3N21 ; C[C@@H]1CN=C2N(Cc3cnn(C)c3)C(=O)c3cc(S(=O)(=O)NC4(C)CC4)cc(-c4ccc(F)cn4)c3N21]

To a stirred solution of (1R)-9-bromo-1-methyl-N-(1-methylcyclopropyl)-4-[(1-methylpyrazol-4-yl)methyl]-5-oxo-1H,2H-imidazo[1,2-a]quinazoline-7-sulfonamide (80 mg, 0.16 mmol) in anhydrous DMF (2 mL) was added 5-fluoro-2-(tributylstannyl)pyridine (91.3 mg, 0.24 mmol) and P(t-Bu)₃.HBF₄ (1.5 mg, 0.016 mmol) followed by catalytic amount of P(t-Bu)₃ Pd G3 (15.2 mg, 0.016 mmol) at room temperature. The resulting mixture was stirred at 80° C. overnight under nitrogen atmosphere. The reaction mixture was purified by Prep-HPLC with the following conditions (Column: Xselect CSH F-Phenyl OBD column 30×250 mm, 5 m; Mobile Phase A: water (0.1% FA), Mobile Phase B: ACN; Flow rate: 60 mL/min; Gradient: 2% B to 20% B in 10 min; Wave Length: 254 nm/220 nm; RT1(min): 12.77) to afford (1R)-9-(5-fluoropyridin-2-yl)-1-methyl-N-(1-methylcyclopropyl)-4-[(1-methylpyrazol-4-yl)methyl]-5-oxo-1H,2H-imidazo[1,2-a]quinazoline-7-sulfonamide (8.4 mg, 10%). LCMS (ESI) m/z: 524.15 [M+H]⁺. ¹H NMR (400 MHz, DMSO-d₆) δ 8.71 (d, J=2.8 Hz, 1H), 8.38 (dd, J=2.4, 1.1 Hz, 1H), 8.09 (s, 1H), 7.92 (td, J=8.4, 2.9 Hz, 1H), 7.86-7.82 (m, 1H), 7.81 (1; J=1.6 Hz, 1H), 7.70 (s, 1H), 7.42 (s, 1H), 5.07-4.90 (m, 2H), 3.77 (s, 3H), 3.74-3.68 (m, 1H), 3.58 (q, J=7.2 Hz, 1H), 3.28 (d, J=1.7 Hz, 1H), 1.08 (s, 3H), 0.74 (d, J=6.2 Hz, 3H), 0.61 (q, J=3.6 Hz, 2H), 0.40 (dd, J=5.2, 2.0 Hz, 2H).

Representative Synthesis Route 6: Example 8: (R)-4-((5-cyclopropyl-3-methylisoxazol-4-yl)methyl)-1-methyl-N-(1-methylcyclopropyl)-5-oxo-1,2,4,5-tetrahydroimidazo[1,2-a]quinazoline-7-sulfonamide

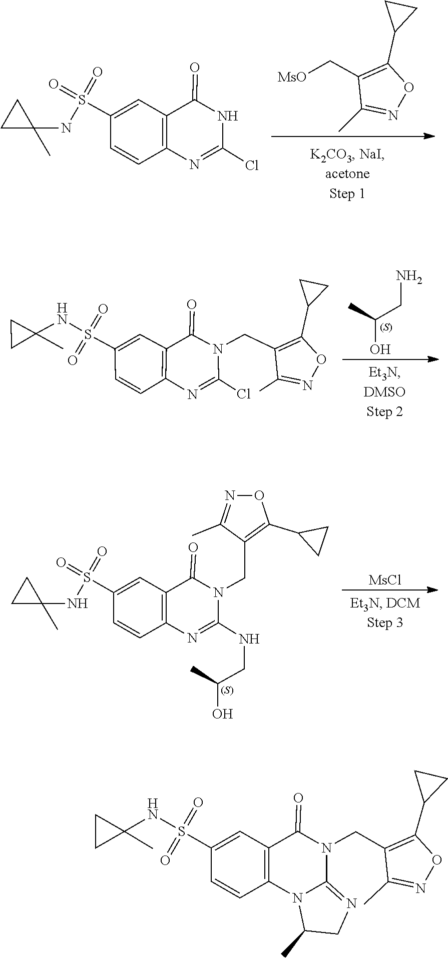

Step 1: To a stirred mixture of 2-chloro-N-(1-methylcyclopropyl)-4-oxo-3H-quinazoline-6-sulfonamide (358 mg, 1.14 mmol) in acetone (5.7 mL) were added 5-cyclopropyl-4-[(mesyloxy)meth yl]-3-methylisoxazole (317 mg, 1.37 mmol), NaI (catalytic) and K₂CO₃ (3 16 mg, 2.28 mmol) at room temperature. The resulting mixture was stirred at room temperature overnight. The reaction mixture was washed with water and brine and extracted with EtOAc. The resulting crude mixture was purified by silica gel column chromatography, eluted with EtOAc/hexane (0-100%) to afford 2-chloro-3-[(5-cyclopropyl-3-methyl-4-isoxazolyl)methyl]-6-(1-methylcyclopropylamino sulfonyl)-3,4-dihydro-4-quinazolinone (247 mg, 48%) as a white solid. LCMS (ESI) m/z: 449 [M+H]⁺.

Synthesis of Example 8 was completed (steps 2 and 3) using procedures similar to those described in Representative Synthesis Route 1 for Example 1, steps 6 and 7 using appropriate starting materials. LCMS (ESI) m/z: 470.00 [M+H]⁺. 1H NMR (400 MHz, DMSO-d₆) δ 8.23 (d, J=2.0 Hz, 1H), 8.02 (s, 1H), 7.93-7.90 (m, 1H), 7.26-7.24 (m, 1H), 5.02-4.91 (m, 2H), 4.74-4.71 (m, 1H), 4.05-4.00 (m, 1H), 3.52-3.48 (m, 1H), 2.48-2.46 (m, 1H), 2.24 (s, 3H), 1.31-1.30 (m, 3H), 1.06 (s, 3H), 0.99-0.84 (m, 4H), 0.60-0.58 (m, 2H), 0.38-0.36 (m, 2H). Representative Synthesis Route 7: Example 10: (R)-1-methyl-4-((1-methyl-1H-pyrazol-4-yl)methyl)-N-(1-methylcyclopropyl)-9-(4-methylpiperazin-1-yl)-5-oxo-1,2,4,5-tetrahy droimidazo[1,2-a]quinazoline-7-sulfonamide

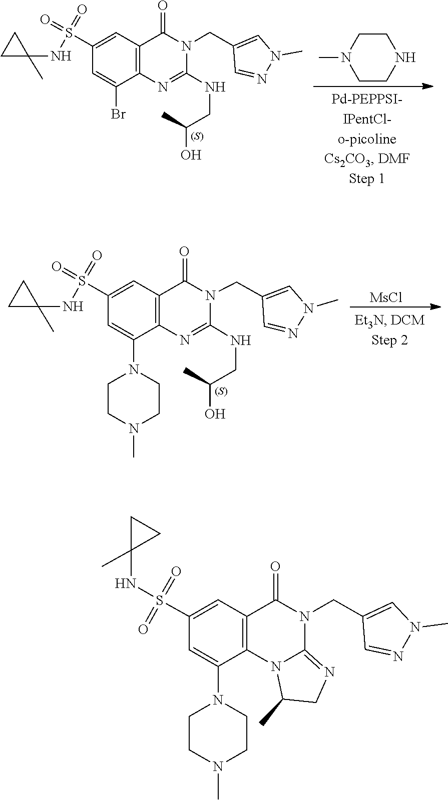

Step 1: A mixture of 8-bromo-2-{[(2S)-2-hydroxypropyl]amino}-N-(1-methylcyclopropyl)-3-[(1-methylpyrazol-4-yl)methyl]-4-oxoquinazoline-6-sulfonamide (120 mg, 0.23 mmol), N-methyl piperazine (34.3 mg, 0.34 mmol), Cs₂CO₃ (148.8 mg, 0.46 mmol) and Pd-PEPPSI-IPentCl 2-methylpyridine (o-picoline) (1.6 mg, 0.002 mmol) in DMF (6 mL) was stirred for 4 h at 120° C. under nitrogen atmosphere. The residue was purified by reversed-phase flash chromatography with the following conditions: column, C18 silica gel; mobile phase, MeCN in water (10 mmol/L NH₄HCO₃), 20% to 50% gradient in 20 min; detector, UV 254 nm. This resulted in 2-{[(2S)-2-hydroxypropyl]amino}-N-(1-methylcyclopropyl)-8-(4-methylpiperazin-1-yl)-3-[(1-methylpyrazol-4-yl)methyl]-4-oxoquinazoline-6-sulfonamide (30 mg, 24%) as a yellow solid. LCMS (ESI) m/z: 545 [M+H]⁺.

Synthesis of Example 10 was completed (step 2) using procedures similar to those described in Representative Synthesis Route 1 for Example 1, step 7 using appropriate starting materials. LCMS (ESI) m/z: 527.20 [M+H]⁺. ¹H NMR (400 MHz, DMSO-d₆) δ 8.08 (d, J=2.1 Hz, 1H), 8.03 (s, 1H), 7.76 (d, J=2.4 Hz, 1H), 7.68 (s, 1H), 7.41 (s, 1H), 5.26-5.16 (m, 1H), 5.00-4.89 (m, 2H), 3.92 (dd, J=13.5, 8.6 Hz, 1H), 3.76 (s, 3H), 3.50 (dd, J=13.6, 1.7 Hz, 1H), 3.15 (t, J=10.6 Hz, 1H), 3.05-2.88 (m, 3H), 2.72 (d, J=8.8 Hz, 1H), 2.30 (t, J=7.5 Hz, 2H), 2.25 (s, 3H), 2.20-2.09 (m, 1H), 1.03 (d, J=5.4 Hz, 6H), 0.59 (dd, J=9.9, 7.0 Hz, 2H), 0.41-0.33 (in, 2H).

The following compounds in Table 7 were prepared using procedures similar to those described in Representative Synthesis Route 7 for Example 10 using appropriate starting

TABLE 7

| Example | | |
|---|---|---|
| No. | Name | [M + H]⁺ |
| 13 | (R)-1-methyl-4-((1-methyl-1H-pyrazol-4-yl)methyl)-N-(1- | 513.10 |
| | methylcyclopropyl)-5-oxo-9-(piperazin-1-yl)-1,2,4,5- | |
| | tetrahydroimidazo[1,2-a]quinazoline-7-sulfonamide | |
| 33 | (R)-4-(1,4-dimethyl-7-(N-(1-methylcyclopropyl)sulfamoyl)-5- | 504.35 |
| | oxo-1,2,4,5-tetrahydroimidazo[1,2-a]quinazolin-9-yl)-N,N- | |
| | dimethylpiperazine-1-carboxamide | |
| 37 | (R)-N,N-dimethyl-1-(1-methyl-4-((1-methyl-1H-pyrazol-4- | 583.25 |
| | yl)methyl)-7-(N-(1-methylcyclopropyl)sulfamoyl)-5-oxo- | |
| | 1,2,4,5-tetrahydroimidazo[1,2-a]quinazolin-9-yl)piperidine-4- | |
| | carboxamide | |
| 46 | N,N-dimethyl-4-(2,2,4-trimethyl-7-(N-(1- | 518.25 |
| | methylcyclopropyl)sulfamoyl)-5-oxo-1,2,4,5- | |
| | tetrahydroimidazo[1,2-a]quinazolin-9-yl)piperazine-1- | |
| | carboxamide | |
| 53 | (R)-9-((3S,5S)-3,5-dimethylpiperazin-1-yl)-1-methyl-4-((1- | 541.25 |
| | methyl-1H-pyrazol-4-yl)methyl)-N-(1-methylcyclopropyl)-5- | |
| | oxo-1,2,4,5-tetrahydroimidazo[1,2-a]quinazoline-7- | |
| | sulfonamide | |
| 54 | 9-((3S,5S)-3,5-dimethylpiperazin-1-yl)-2,2,4-trimethyl-N-(1- | 475.15 |
| | methylcyclopropyl)-5-oxo-1,2,4,5-tetrahydroimidazo[1,2- | |
| | a]quinazoline-7-sulfonamide | |
| 57 | (R,E)-9-(4-(4-(4-methoxyphenyl)-4-oxobut-2-enoyl)piperazin- | 621.15 |
| | 1-yl)-1,4-dimethyl-N-(1-methylcyclopropyl)-5-oxo-1,2,4,5- | |
| | tetrahydroimidazo[1,2-a]quinazoline-7-sulfonamide | |
| 71 | (R)-9-((R)-3-aminopyrrolidin-1-yl)-1-methyl-4-((1-methyl- | 513.10 |
| | 1H-pyrazol-4-yl)methyl)-N-(1-methylcyclopropyl)-5-oxo- | |
| | 1,2,4,5-tetrahydroimidazo[1,2-a]quinazoline-7-sulfonamide | |
| 74 | (R)-9-(3-amino-3-methylazetidin-1-yl)-1-methyl-4-((1- | 513.10 |
| | methyl-1H-pyrazol-4-yl)methyl)-N-(1-methylcyclopropyl)-5- | |
| | oxo-1,2,4,5-tetrahydroimidazo[1,2-a]quinazoline-7- | |
| | sulfonamide | |
| 75 | (R)-9-((S)-3-aminopyrrolidin-1-yl)-1-methyl-4-((1-methyl- | 513.20 |
| | 1H-pyrazol-4-yl)methyl)-N-(1-methylcyclopropyl)-5-oxo- | |
| | 1,2,4,5-tetrahydroimidazo[1,2-a]quinazoline-7-sulfonamide | |
| 78 | (R)-1-methyl-4-((1-methyl-1H-pyrazol-4-yl)methyl)-N-(1- | 527.25 |
| | methylcyclopropyl)-9-((R)-3-methylpiperazin-1-yl)-5-oxo- | |
| | 1,2,4,5-tetrahydroimidazo[1,2-a]quinazoline-7-sulfonamide | |
| 82 | (R)-1-methyl-4-((1-methyl-1H-pyrazol-4-yl)methyl)-N-(1- | 527.15 |
| | methylcyclopropyl)-9-((S)-3-methylpiperazin-1-yl)-5-oxo- | |
| | 1,2,4,5-tetrahydroimidazo[1,2-a]quinazoline-7-sulfonamide | |
| 88 | (R)-9-((3R,5R)-3,5-dimethylpiperazin-1-yl)-1-methyl-4-((1- | 541.15 |
| | methyl-1H-pyrazol-4-yl)methyl)-N-(1-methylcyclopropyl)-5- | |
| | oxo-1,2,4,5-tetrahydroimidazo[1,2-a]quinazoline-7- | |
| | sulfonamide | |
| 123 | (R)-1,4-dimethyl-N-(1-methylcyclopropyl)-5-oxo-9-(2-oxa-7- | 474.30 |
| | azaspiro[3.5]nonan-7-yl)-1,2,4,5-tetrahydroimidazo[1,2- | |
| | a]quinazoline-7-sulfonamide | |

Dioxane was used instead of DMF for examples 33, 46, 53, 123, while dioxane/DMF (10:1) was used for examples 71, 74, 75, 78, 82, 88.

Representative Synthesis Route 8: Example 22: (R)-1-methyl-4-((1-methyl-1H-pyrazol-4-yl)methyl)-N-(1-methylcyclopropyl)-9-(1-methylpiperidin-4-yl)-5-oxo-1,2,4,5-tetrahydroimidazo[1,2-a]quinazoline-7-sulfonamide

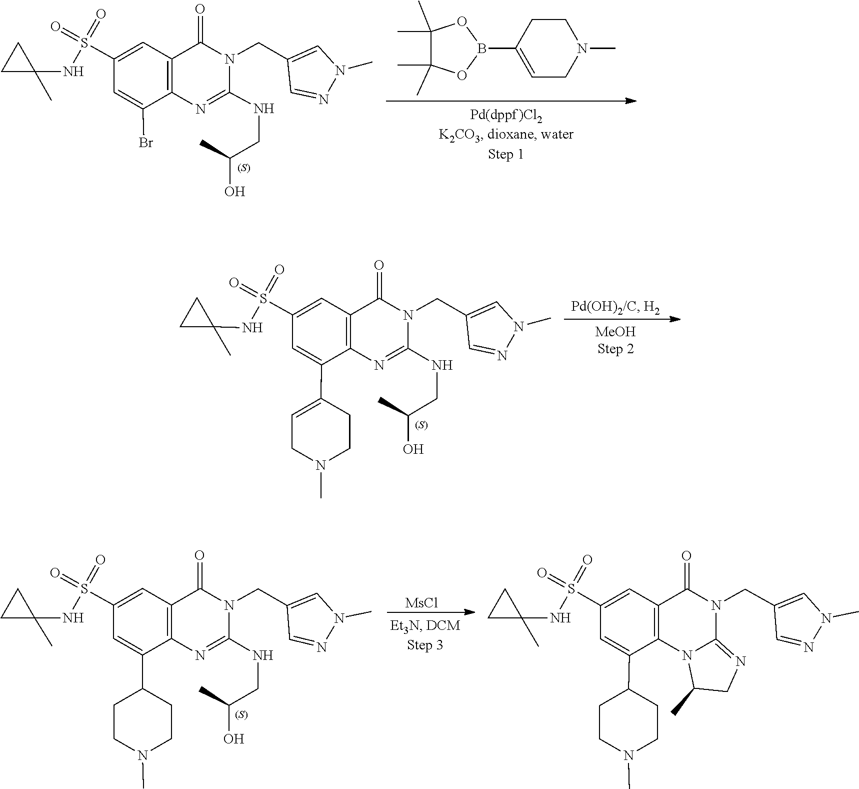

Step 1: A solution of 8-bromo-2-{[(2S)-2-hydroxypropyl]amino}-N-(1-methylcyclopropyl)-3-[(1-methylpyrazol-4-yl)methyl]-4-oxoquinazoline-6-sulfonamide (100 mg, 0.19 mmol), 1-methyl-4-(4,4,5,5-tetramethyl-1,3,2-dioxaborolan-2-yl)-3,6-dihydro-2H-pyridine (63.7 mg, 0.28 mmol), K₂CO₃ (52.6 mg, 0.38 mmol) and Pd(dppf)Cl₂ (13.9 mg, 0.019 mmol) in 1,4-dioxane (10 mL) and H₂O (1 mL) was stirred for 2 h at 90° C. under nitrogen atmosphere. The resulting mixture was concentrated under reduced pressure. The residue was purified by silica gel column chromatography, eluted with CH₂Cl₂/MeOH (8:1) to afford 2-{[(2S)-2-hydroxypropyl]amino}-8-(1-methyl-3,6-dihydro-2H-pyridin-4-yl)-N-(1-methylcyclopropyl)-3-[(1-methylpyrazol-4-yl)methyl]-4-oxoquinazoline-6-sulfonamide (50 mg, 48%) as a yellow solid. LCMS (ESI) m/z: 542 [M+H]⁺.

Step 2: A solution of 2-{[(2S)-2-hydroxypropyl]amino}-8-(1-methyl-3,6-dihydro-2H-pyridin-4-yl)-N-(1-methylcyclopropyl)-3-[(1-methylpyrazol-4-yl)methyl]-4-oxoquinazoline-6-sulfonamide (50 mg, 0.092 mmol) and Pd(OH)₂/C (13 mg, 0.092 mmol) in MeOH (10 mL) was stirred for 4 h at 40° C. under hydrogen atmosphere. The resulting mixture was filtered, the filter cake was washed with MeOH (3×5 mL). The filtrate was concentrated under reduced pressure. This resulted in 2-{[(2S)-2-hydroxypropyl]amino}-N-(1-methylcyclopropyl)-8-(1-methylpiperidin-4-yl)-3-[(1-methylpyrazol-4-yl)methyl]-4-oxoquinazoline-6-sulfonamide (30 mg) as a yellow solid. LCMS (ESI) m/z: 544 [M+H]⁺.

Synthesis of Example 22 was completed (step 3) using procedures similar to those described in Representative Synthesis Route 1 for Example 1, step 7 using appropriate starting materials. LCMS (ESI) m/z: 526.20 [M+H]⁺. 1H NMR (400 MHz, DMSO-d₆) δ 8.54 (s, 1H), 8.42 (d, J=2.6 Hz, 1H), 7.95 (d, J=2.4 Hz, 1H), 7.68 (s, 1H), 7.54 (s, 1H), 5.11-4.95 (m, 3H), 4.07 (dd, J=13.2, 8.3 Hz, 1H), 3.82 (s, 3H), 3.60 (dd, J=27.6, 12.4 Hz, 2H), 3.37 (d, J=12.2 Hz, 1H), 3.28-3.21 (m, 1H), 3.06-2.95 (m, 2H), 2.75 (s, 3H), 2.26 (d, J=8.4 Hz, 2H), 2.11 (d, J=14.4 Hz, 1H), 1.56 (q, J=13.2 Hz, 1H), 1.20 (d, J=6.2 Hz, 3H), 1.12 (s, 3H), 0.70 (q, J=3.4 Hz, 2H), 0.47-0.40 (m, 2H).

Representative Synthesis Route 9: Example 27: (R)-9-(4-hydroxypiperidin-1-yl)-1-methyl-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

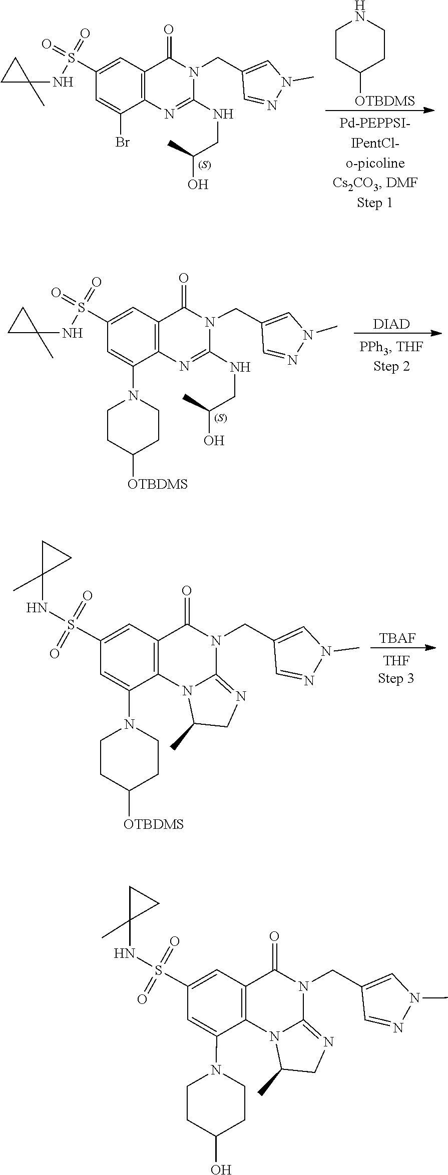

Step 1: A mixture of 8-bromo-2-{[(2S)-2-hydroxypropyl]amino}-N-(1-methylcyclopropyl)-3-[(1-methylpyrazol-4-yl)methyl]-4-oxoquinazoline-6-sulfonamide (150 mg, 0.28 mmol), 4-[(tert-butyldimethylsilyl)oxy]piperidine (92.2 mg, 0.43 mmol), Cs₂CO₃ (186 mg, 0.57 mmol) and Pd-PEPPSI-IPentCl 2-methylpyridine (o-picoline) (20.8 mg, 0.025 mmol) in DMF (5 mL) was stirred for 4 h at 120° C. under nitrogen atmosphere. The residue was purified by reversed-phase flash chromatography with the following conditions: column, C18 silica gel; mobile phase, MeCN in water (10 mmol/L NH₄HCO₃), 50% to 70% gradient in 10 min; detector, UV 254 nm. This resulted in 8-{4-[(tert-butyldimethylsilyl)oxy]piperidin-1-yl}-2-{[(2S)-2-hydroxypropyl]amino}-N-(1-methylcyclopropyl)-3-[(1-methylpyrazol-4-yl)methyl]-4-oxoquinazoline-6-sulfonamide (60 mg, 32%) as a yellow solid. LCMS (ESI) m/z: 660 [M+H]⁺.

Step 2: A solution of 8-{4-[(tert-butyldimethylsilyl)oxy]piperidin-1-yl}-2-{[(2S)-2-hydroxypropyl]amino}-N-(1-methylcyclopropyl)-3-[(1-methylpyrazol-4-yl)methyl]-4-oxoquinazoline-6-sulfonamide (50 mg, 0.076 mmol), DIAD (18.4 mg, 0.091 mmol) and PPh₃ (39.7 mg, 0.15 mmol) in THF (5 mL) was stirred for 16 h at 0° under nitrogen atmosphere. The resulting mixture was extracted with EtOAc (2×50 mL). The combined organic layers were washed with brine (2×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% FA), 10% to 50% gradient in 10 min; detector, UV 254 nm. This resulted in (1R)-9-{4-[(tert-butyldimethylsilyl)oxy]piperidin-1-yl}-1-methyl-N-(1-methylcyclopropyl)-4-[(1-methylpyrazol-4-yl)methyl]-5-oxo-1H,2H-imidazo[1,2-a]quinazoline-7-sulfonamide (30 mg, 62%) as a white solid. LCMS (ESI) m/z: 642 [M+H]⁺.

Step 3: A solution of (1R)-9-{4-[(tert-butyldimethylsilyl)oxy]piperidin-1-yl}-1-methyl-N-(1-methylcyclopropyl)-4-[(1-methylpyrazol-4-yl)methyl]-5-oxo-1H,2H-imidazo[1,2-a]quinazoline-7-sulfonamide (30 mg, 0.047 mmol) and TBAF (24.4 mg, 0.094 mmol) in THF (5 mL) was stirred for 1 h at room temperature. The crude product was purified by Prep-HPLC with the following conditions (Column: XBridge Prep Phenyl OBD Column 19×250 mm; Mobile Phase A: water (10 mmol/L NH₄HCO₃), Mobile Phase B: CAN; Flow rate: 60 mL/min; Gradient: 15% B to 30% B in 10 min; Wave Length: 254 nm/220 nm; RT1(min): 12.88) to afford (1R)-9-(4-hydroxypiperidin-1-yl)-1-methyl-N-(1-methylcyclopropyl)-4-[(1-methylpyrazol-4-yl)methyl]-5-oxo-1H,2H-imidazo[1,2-a]quinazoline-7-sulfonamide(6.3 mg, 25%). LCMS (ESI) m/z: 528.25 [M+H]⁺. 1H NMR (400 MHz, DMSO-d₆) δ 8.10-7.98 (m, 2H), 7.84-7.66 (m, 2H), 7.41 (s, 1H), 5.28-5.18 (m, 1H), 5.01-4.89 (m, 2H), 4.73 (dd, J=79.9, 3.6 Hz, 1H), 3.92 (td, J=12.8, 11.8, 8.8 Hz, 1H), 3.77 (s, 3H), 3.60-3.45 (m, 2H), 3.08-3.00 (m, 2H), 2.78 (dd, J=23.3, 11.2 Hz, 1H), 2.25 (dd, J=13.5, 10.8 Hz, 1H), 2.05-1.87 (m, 1H), 1.81 (d, J=9.6 Hz, 1H), 1.70-1.44 (m, 2H), 1.09-1.01 (m, 6H), 0.61 (t, J=6.4 Hz, 2H), 0.38 (t, J=2.2 Hz, 2H).

Representative Synthesis Route 10: Example 32: (R)-9-(4-fluoro-2-methylphenyl)-1-methyl-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

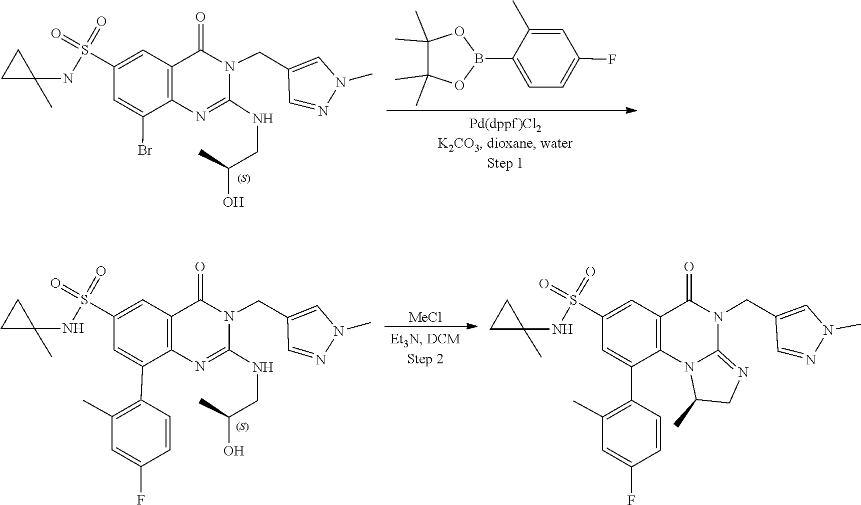

Step 1: A mixture of 8-bromo-2-{[(2S)-2-hydroxypropyl]amino}-N-(1-methylcyclopropyl)-3-[(1-methylpyrazol-4-yl)methyl]-4-oxoquinazoline-6-sulfonamide (120 mg, 0.23 mmol), 2-(4-fluoro-2-methylphenyl)-4,4,5,5-tetramethyl-1,3,2-dioxaborolane (81 mg, 0.34 mmol), K₂CO₃(63.1 mg, 0.46 mmol) and Pd(dppf)Cl₂ (16.7 mg, 0.023 mmol) in 1,4-dioxane (5 mL) and H₂O (0.5 mL) was stirred for 2 h at 90° C. under nitrogen atmosphere. The resulting mixture was concentrated under reduced pressure. The residue was purified by silica gel column chromatography, eluted with CH₂Cl₂/MeOH (8:1) to afford 8-(4-fluoro-2-methylphenyl)-2-{[(2S)-2-hydroxypropyl]amino}-N-(1-methylcyclopropyl)-3-[(1-methylpyrazol-4-yl)methyl]-4-oxoquinazoline-6-sulfonamide (50 mg, 39%) as a yellow solid. LCMS (ESI) m/z: 555 [M+H]⁺. Synthesis of Example 32 was completed (step 2) using procedures similar to those described in Representative Synthesis Route 1 for Example 1, step 7 using appropriate starting materials. LCMS (ESI) m/z: 537.10 [M+H]⁺. ¹H NM/R (400 1VUz, DMSO-d₆) δ 8.35 (dd, J=7.8, 2.4 Hz, 1H), 8.05 (d, J=8.8 Hz, 1H), 7.69 (s, 1H), 7.63 (dd, J=4.4, 2.4 Hz, 1H), 7.55 (dd, J=8.2, 6.0 Hz, 1H), 7.42 (s, 1H), 7.38-7.18 (m, 2H), 5.02-4.90 (m, 2H), 3.77 (s, 3H), 3.69-3.61 (m, 1H), 3.41 (q, J=6.4, 6.0 Hz, 1H), 3.28 (d, J=13.4 Hz, 1H), 2.33 (s, 1H), 1.99 (s, 2H), 1.07 (d, J=9.2 Hz, 3H), 0.73 (dd, J=10.0, 5.8 Hz, 3H), 0.61 (dd, J=9.6, 6.8 Hz, 2H), 0.38 (d, J=2.4 Hz, 2H).

The following compounds in Table 8 were prepared using procedures similar to those described in Representative Synthesis Route 10 for Example 32 using appropriate starting materials.

TABLE 8

| Example | | |
|---|---|---|
| No. | Name | [M + H]⁺ |
| 50 | N-methyl-5-((7aS,10aR)-6-methyl-3-(N-(1- | 509.10 |
| | methylcyclopropyl)sulfamoyl)-5-oxo-6,7a,8,9,10,10a- | |
| | hexahydro-5H-cyclopenta[4,5]imidazo[1,2-a]quinazolin-1- | |
| | yl)picolinamide | |
| 52 | (7aS,10aR)-1-(1-(cyclopropanecarbonyl)-1,2,3,6- | 524.15 |
| | tetrahydropyridin-4-yl)-6-methyl-N-(1-methylcyclopropyl)-5- | |
| | oxo-6,7a,8,9,10,10a-hexahydro-5H- | |
| | cyclopenta[4,5]imidazo[1,2-a]quinazoline-3-sulfonamide | |
| 76 | (R)-1-ethynyl-9-(1-methyl-1,2,3,6-tetrahydropyridin-4-yl)-4- | 534.10 |
| | ((1-methyl-1H-pyrazol-4-yl)methyl)-N-(1- | |
| | methylcyclopropyl)-5-oxo-1,2,4,5-tetrahydroimidazo[1,2- | |
| | a]quinazoline-7-sulfonamide | |
| 79 | (R)-1-ethynyl-4-((1-methyl-1H-pyrazol-4-yl)methyl)-N-(1- | 520.15 |
| | methylcyclopropyl)-5-oxo-9-(1,2,3,6-tetrahydropyridin-4-yl)- | |
| | 1,2,4,5-tetrahydroimidazo[1,2-a]quinazoline-7-sulfonamide | |
| 80 | (R)-1-methyl-4-((1-methyl-1H-pyrazol-4-yl)methyl)-N-(1- | 510.10 |
| | methylcyclopropyl)-5-oxo-9-(1,2,3,6-tetrahydropyridin-4-yl)- | |
| | 1,2,4,5-tetrahydroimidazo[1,2-a]quinazoline-7-sulfonamide | |

Representative Synthesis Route 11: Example 35 and Example 91: (R)-1-((1-methyl-1H-pyrazol-4-yl)ethynyl)-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide and (S′)-1-((1-methyl-1H-pyrazol-4-yl)ethynyl)-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

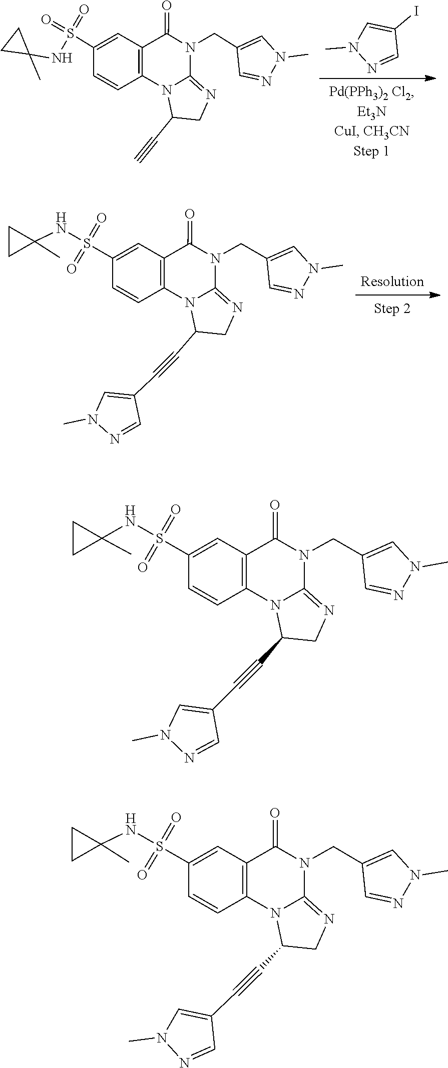

Step 1: To a stirred mixture of 1-ethynyl-N-(1-methylcyclopropyl)-4-[(1-methylpyrazol-4-yl)methyl]-5-oxo-1H,2H-imidazo[1,2-a]quinazoline-7-sulfonamide (40 mg, 0.091 mmol) and 4-iodo-1-methylpyrazole (18.9 mg, 0.091 mmol) in TEA (4 mL) and ACN (4 mL) were added CuI (1.74 mg, 0.009 mmol) and Pd(PPh₃)₂Cl₂ (6.40 mg, 0.009 mmol) at room temperature under nitrogen atmosphere. The resulting mixture was stirred for 1 h at 80° C., concentrated under reduced pressure. The residue was purified by silica gel column chromatography, eluted with PE/EA (1:1) to afford N-(1-methylcyclopropyl)-1-[2-(1-methylpyrazol-4-yl)ethynyl]-4-[(ta1-methylpyrazol-4-yl)methyl]-5-oxo-1H,2H-imidazo[1,2-a]quinazoline-7-sulfonamide (40 mg, 84%). LCMS (ESI) m/z: 519 [M+H]⁺.

Step 2: The crude product was purified by Prep-HPLC with the following conditions (Column: CHIRAL ART Cellulose-SB, 2×25 cm, 5 m; Mobile Phase A: Hex (0.5% 2M NH₃-MeOH), Mobile Phase B: EtOH. HPLC; Flow rate: 20 mL/min; Gradient: isocratic 30; Wave Length: 240/210 nm; RT1(min): 10.15; RT2 (min): 12.55; to afford (1R)-N-(1-methylcyclopropyl)-1-[2-(1-methylpyrazol-4-yl)ethynyl]-4-[(1-methylpyrazol-4-yl)methyl]-5-oxo-1H,2H-imidazo[1,2-a]quinazoline-7-sulfonamide (1.1 mg, 4%) and (1S)-N-(1-methylcyclopropyl)-1-[2-(1-methylpyrazol-4-yl)ethynyl]-4-[(1-methylpyrazol-4-yl)methyl]-5-oxo-1H,2H-imidazo[1,2-a]quinazoline-7-sulfonamide (0.6 mg, 2%). LCMS (ESI) m/z: 519.10 [M+H]⁺. ¹H NMR (400 MHz, CD₃OD) δ 8.44 (d, J=2.4 Hz, 1H), 8.06-8.16 (m, 1H), 7.74 (s, 1H), 7.69 (s, 1H), 7.56 (s, 1H), 7.52 (s, 1H), 7.39-7.37 (d, J=8.8 Hz, 1H), 5.53-5.48 (m, 1H), 5.03 (s, 2H), 4.41-4.34 (m, 1H), 4.15-4.05 (m, 1H), 3.83 (s, 6H), 1.14 (s, 3H), 0.71-0.68 (m, 2H), 0.43-0.42 (m, 2H).

The following compounds in Table 9 were prepared using procedures similar to those described in Representative Synthesis Route 11 for Example 35 using appropriate starting materials.

TABLE 9

| Example | | |
|---|---|---|
| No. | Name | [M + H]⁺ |
| 65 | (R)-1-((1-methyl-1H-pyrazol-3-yl)ethynyl)-4-((1-methyl-1H- | 519.20 |
| | pyrazol-4-yl)methyl)-N-(1-methylcyclopropyl)-5-oxo-1,2,4,5- | |
| | tetrahydroimidazo[1,2-a]quinazoline-7-sulfonamide | |
| 66 | (R)-1-((1-methyl-1H-1,2,4-triazol-3-yl)ethynyl)-4-((1-methyl- | 520.00 |
| | 1H-pyrazol-4-yl)methyl)-N-(1-methylcyclopropyl)-5-oxo- | |
| | 1,2,4,5-tetrahydroimidazo[1,2-a]quinazoline-7-sulfonamide | |
| 67 | (R)-4-((1-methyl-1H-pyrazol-4-yl)methyl)-N-(1- | 516.20 |
| | methylcyclopropyl)-5-oxo-1-(pyridin-2-ylethynyl)-1,2,4,5- | |
| | tetrahydroimidazo[1,2-a]quinazoline-7-sulfonamide | |
| 68 | (R)-4-((1-methyl-1H-pyrazol-4-yl)methyl)-N-(1- | 517.25 |
| | methylcyclopropyl)-5-oxo-1-(pyrimidin-2-ylethynyl)-1,2,4,5- | |
| | tetrahydroimidazo[1,2-a]quinazoline-7-sulfonamide | |
| 77 | (R)-4-((1-methyl-1H-pyrazol-4-yl)methyl)-N-(1- | 517.15 |
| | methylcyclopropyl)-5-oxo-1-(pyrazin-2-ylethynyl)-1,2,4,5- | |
| | tetrahydroimidazo[1,2-a]quinazoline-7-sulfonamide | |
| 83 | (R)-1-((1-methyl-1H-imidazol-2-yl)ethynyl)-4-((1-methyl-1H- | 519.10 |
| | pyrazol-4-yl)methyl)-N-(1-methylcyclopropyl)-5-oxo-1,2,4,5- | |
| | tetrahydroimidazo[1,2-a]quinazoline-7-sulfonamide | |

Representative Synthesis Route 12: Example 41: (R)-8-methyl-5-((1-methyl-1H-pyrazol-4-yl)methyl)-N-(1-methylcyclopropyl)-4-oxo-4,5,7,8-tetrahydroimidazo[1,2-a]thieno[3,2-e]pyrimidine-2-sulfonamide

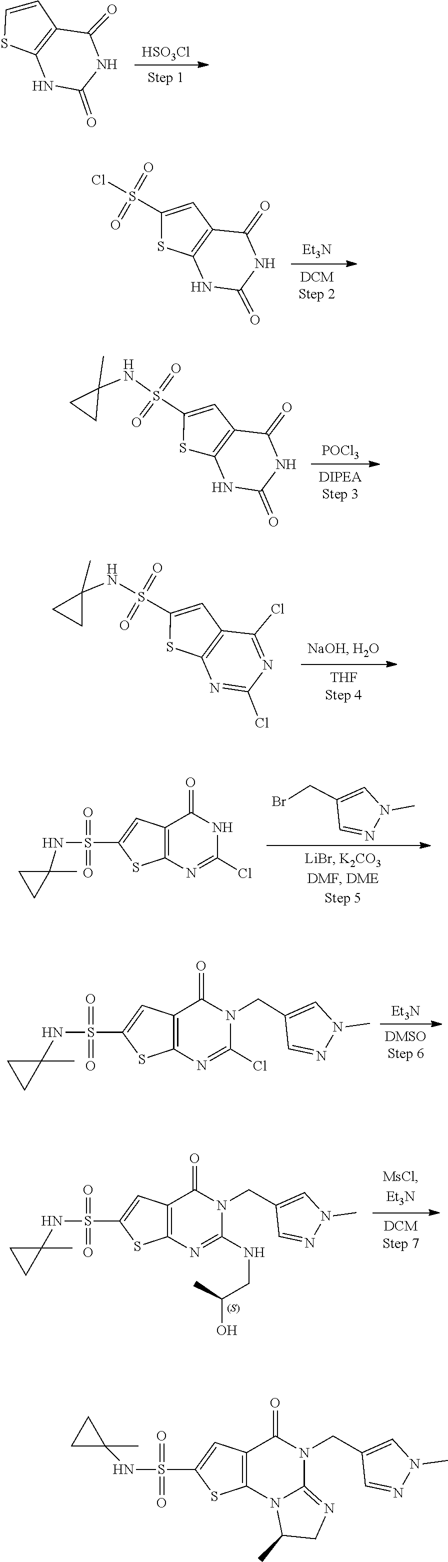

Step 1: Into a 50 mL round-bottom flask were added 1H,3H-thieno[2,3-d]pyrimidine-2,4-dione (2 g, 11.9 mmol) and HSO₃Cl (30 mL) at 0° C. The resulting mixture was stirred for 2 h at 40° C. under nitrogen atmosphere. The reaction was quenched by the addition of water/ice (50 mL) at 0° C. The resulting mixture was extracted with EtOAc (2×100 mL). The combined organic layers were washed with brine (100 mL), dried over anhydrous Na₂SO₄. After filtration, the filtrate was concentrated under reduced pressure. The crude resulting mixture was used in the next step directly without further purification. LCMS (ESI) m/z: 267 [M+H]⁺.

Step 2: A mixture of 2,4-dioxo-1H,3H-thieno[2,3-d]pyrimidine-6-sulfonyl chloride (1.5 g, 5.62 mmol) in TEA (10 mL, 16.8 mmol) was stirred for 12 h at 100° C. under nitrogen atmosphere. The resulting mixture was concentrated under reduced pressure. The crude resulting mixture was used in the next step directly without further purification. LCMS (ESI) m/z: 302 [M+H]⁺.

Step 3: To a stirred mixture of N-(1-methylcyclopropyl)-2,4-dioxo-1H,3H-thieno[2,3-d]pyrimidine-6-sulfonamide (1 g, 3.32 mmol) in POCl₃ (30 mL, 3.32 mmol) was added DIEA (1.29 g, 9.96 mmol) dropwise at room temperature under nitrogen atmosphere. The resulting mixture was stirred for 12 h at 100° C. under nitrogen atmosphere. The resulting mixture was concentrated under reduced pressure. The reaction was quenched with water at room temperature and was extracted with EtOAc (2×100 mL). The combined organic layers were washed with brine 100 mL), dried over anhydrous Na₂SO₄. After filtration, the filtrate was concentrated under reduced pressure. The residue was purified by silica gel column chromatography, eluted with PE/EA (1:3) to afford 2,4-dichloro-N-(1-methylcyclopropyl)thieno[2,3-d]pyrimidine-6-sulfonamide (600 mg, 53%) as a yellow oil.

Step 4: A mixture of 2,4-dichloro-N-(1-methylcyclopropyl)thieno[2,3-d]pyrimidine-6-sulfonamide (500 mg, 1.48 mmol) and NaOH (295.6 mg, 7.39 mmol) in H₂O (10 mL) and THF (10 mL) was stirred for 2 h at room temperature under nitrogen atmosphere. The reaction was quenched with water at room temperature. The resulting mixture was extracted with EtOAc (2×100 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (1:1) to afford 2-chloro-N-(1-methylcyclopropyl)-4-oxo-3H-thieno[2,3-d]pyrimidine-6-sulfonamide (400 mg, 85%) as a yellow oil. LCMS (ESI) m/z: 320 [M+H]⁺.

Step 5: To a stirred mixture of 2-chloro-N-(1-methylcyclopropyl)-4-oxo-3H-thieno[2,3-d]pyrimidine-6-sulfonamide (300 mg, 0.94 mmol) and 4-(bromomethyl)-1-methylpyrazole (164.2 mg, 0.94 mmol) in DME (12 mL) and DMF (3 mL) were added LiBr (81.5 mg, 0.94 mmol) and K₂CO₃ (518.6 mg, 3.75 mmol) in portions at room temperature under nitrogen atmosphere. The resulting mixture was stirred for 2 h at room temperature under nitrogen atmosphere. The reaction was quenched with water at room temperature. The resulting mixture was extracted with EtOAc (2×150 mL). The combined organic layers were washed with brine (150 mL), dried over anhydrous Na₂SO₄. After filtration, the filtrate was concentrated under reduced pressure. The crude resulting mixture was used in the next step directly without further purification. LCMS (ESI) m/z: 414 [M+H]⁺.

Step 6: A mixture of 2-chloro-N-(1-methylcyclopropyl)-3-[(1-methylpyrazol-4-yl)methyl]-4-oxothieno[2,3-d]pyrimidine-6-sulfonamide (180 mg, 0.43 mmol) and (2S)-1-aminopropan-2-ol (98 mg, 1.30 mmol) in DMSO (15 mL) was stirred for 2 h at room temperature under nitrogen atmosphere. The reaction was quenched with water at room temperature. The resulting mixture was extracted with EtOAc (2×100 mL). The combined organic layers were washed with brine (100 mL), dried over anhydrous Na₂SO₄. After filtration, the filtrate was concentrated under reduced pressure. The residue was purified by silica gel column chromatography, eluted with CH₂Cl₂/MeOH (9:1) to afford (S)-2-((2-hydroxypropyl)amino)-3-((1-methyl-1H-pyrazol-4-yl)methyl)-N-(1-methylcyclopropyl)-4-oxo-3,4-dihydrothieno[2,3-d]pyrimidine-6-sulfonamide (170 mg, 86%) as a yellow oil. LCMS (ESI) m/z: 453 [M+H]⁺.

Step 7: To a stirred mixture of (S)-2-((2-hydroxypropyl)amino)-3-((1-methyl-1H-pyrazol-4-yl)methyl)-N-(1-methylcyclopropyl)-4-oxo-3,4-dihydrothieno[2,3-d]pyrimidine-6-sulfonamide (100 mg, 0.22 mmol) and MsCl (152 mg, 1.33 mmol) in DCM (30 mL) was added TEA (111.8 mg, 1.1 mmol) dropwise at room temperature under nitrogen atmosphere. The resulting mixture was stirred for 2 h at room temperature under nitrogen atmosphere. The resulting mixture was concentrated under reduced pressure. The reaction was quenched with water at room temperature. The resulting mixture was extracted with EtOAc (2×100 mL). The combined organic layers were washed with brine (100 mL), dried over anhydrous Na₂SO₄. After filtration, the filtrate was concentrated under reduced pressure. The crude product was purified by Prep -HPLC with the following conditions (Column: YMC-Actus Triart C18 ExRS 30*150 mm, 5 m; Mobile Phase A: water (10 mmol/L NH₄HCO₃), Mobile Phase B: ACN; Flow rate: 60 mL/min mL/min; Gradient: 21% B to 38.5% B in 10 min; Wave Length: 254 nm/220 nm; RT1(min): 10.25) to afford (R)-8-methyl-5-((1-methyl-1H-pyrazol-4-yl)methyl)-N-(1-methylcyclopropyl)-4-oxo-4,5,7,8-tetrahydroimidazo[1,2-a]thieno[3,2-e]pyrimidine-2-sulfonamide (2.3 mg, 2%). LCMS (ESI) m/z: 435.10 [M+H]⁺. 1H NMR (400 MHz, CD₃OD) δ 7.65-7.64 (d, J=4.8 Hz, 2H), 7.52 (s, 1H), 4.93 (s, 2H), 4.82-4.80 (m, 1H), 4.25-4.18 (m, 1H), 3.82 (s, 3H), 3.66-3.59 (m, 1H), 1.49-1.48 (d, 3H), 1.26 (s, 3H), 0.83-0.79 (m, 2H), 0.52-0.47 (m, 2H).

Representative Synthesis Route 13: Example 43 and Example 90: (R)-1-ethynyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide and(S)-1-ethynyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide

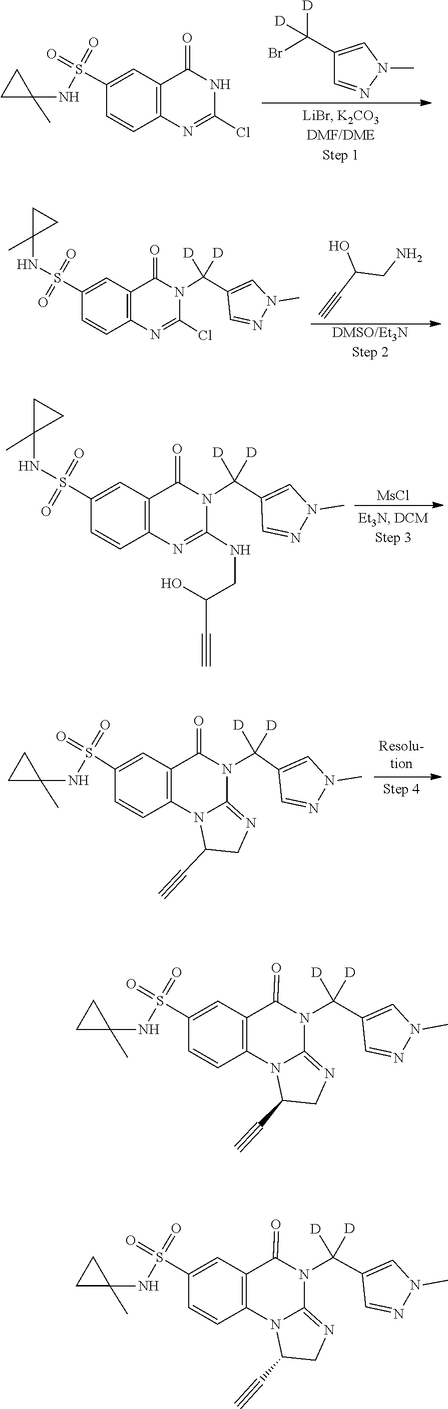

Step 1: To a stirred mixture of 2-chloro-N-(1-methylcyclopropyl)-4-oxo-3H-quinazoline-6-sulfonamide (1 g, 3.19 mmol) and LiBr (276.8 mg, 3.19 mmol) in DME (16 mL) and DMF (4 mL) were added K₂CO₃ (1.32 g, 9.56 mmol) in portions at room temperature. The resulting mixture was stirred for 20 min at 0° C. To the above mixture was added 4-[bromo(2H2)methyl]-1,5-dimethylpyrazole (1.22 g, 6.37 mmol) dropwise over 2 min. The resulting mixture was stirred overnight at room temperature. The reaction was quenched by the addition of water (150 mL) at room temperature. The resulting mixture was extracted with EtOAc (3×160 mL). The combined organic layers were washed with brine (2×10 mL), dried over anhydrous Na₂SO₄.

After filtration, the filtrate was concentrated under reduced pressure. The residue was purified by silica gel column chromatography, eluted with CH₂Cl₂/MeOH (0%~10%) to afford 2-chloro-N-(1-methylcyclopropyl)-3-[(1-methylpyrazol-4-yl)(2H2)methyl]-4-oxoquinazoline-6-sulfonamide (700 mg, 54%) as a light brown solid. LCMS (ESI) m/z: 410 [M+H]⁺.

Step 2: To a stirred mixture of 2-chloro-N-(1-methylcyclopropyl)-3-[(1-methylpyrazol-4-yl)(2H2)methyl]-4-oxoquinazoline-6-sulfonamide (130 mg, 0.32 mmol) and 1-aminobut-3-yn-2-ol (54 mg, 0.63 mmol) in DMSO (6 mL) was added TEA (96.3 mg, 0.95 mmol) dropwise at room temperature. The resulting mixture was stirred for 2 h at room temperature under nitrogen atmosphere. The reaction was quenched by the addition of water (80 mL) at room temperature and extracted with EtOAc (3×100 mL). The combined organic layers were washed with brine (3×10 mL), dried over anhydrous Na₂SO₄. After filtration, the filtrate was concentrated under reduced pressure. This resulted in 2-[(2-hydroxybut-3-yn-1-yl)amino]-N-(1-methylcyclopropyl)-3-[(1-methylpyrazol-4-yl)(2H2)methyl]-4-oxoquinazoline-6-sulfonamide (120 mg) as a brown oil, which was used in the next step directly without further purification. LCMS (ESI) m/z: 459 [M+H]⁺.

Step 3: To a stirred mixture of 2-[(2-hydroxybut-3-yn-1-yl)amino]-N-(1-methylcyclopropyl)-3-[(1-methylpyrazol-4-yl)(2H2)methyl]-4-oxoquinazoline-6-sulfonamide (120 mg, 0.26 mmol) and TEA (79.4 mg, 0.79 mmol) in DCM (15 mL) was added MsCl (59.9 mg, 0.52 mmol) dropwise at room temperature. The resulting mixture was stirred overnight at room temperature.

The reaction was quenched by the addition of water (5 mL) at room temperature. The organic layer was concentrated under reduced pressure. The residue was purified by reversed-phase flash chromatography with the following conditions: column, C18 silica gel; mobile phase, MeCN in water (0.1% FA), 20% to 50% gradient in 40 min; detector, UV 254 nm. This resulted in 1-ethynyl-N-(1-methylcyclopropyl)-4-[(1-methylpyrazol-4-yl)(2H2)methyl]-5-oxo-1H,2H-imidazo[1,2-a]quinazoline-7-sulfonamide (58 mg) as a yellow solid. LCMS (ESI) m/z: 441 [M+H]⁺.

Step 4: The 1-ethynyl-N-(1-methylcyclopropyl)-4-[(1-methylpyrazol-4-yl)(2H2)methyl]-5-oxo-1H,2H-imidazo[1,2-a]quinazoline-7-sulfonamide (50 mg, 0.11 mmol) was purified by Chiral Prep-HPLC with the following conditions (Column: CHIRALPAK-IK, 3×25 mm, 5 m; Mobile Phase A: Hex (10 mM NH₃-MeOH), Mobile Phase B: EtOH. HPLC; Flow rate: 40 mL/min; Gradient: isocratic 45; Wave Length: 220/258 nm; RT1(min): 14.44; RT2 (min): 17.7; to afford (1R)-1-ethynyl-N-(1-methylcyclopropyl)-4-[(1-methylpyrazol-4-yl)(2H2)methyl]-5-oxo-1H,2H-imidazo[1,2-a]quinazoline-7-sulfonamide (21 mg, 42%) and (1S)-1-ethynyl-N-(1-methylcyclopropyl)-4-[(1-methylpyrazol-4-yl)(2H2)methyl]-5-oxo-1H,2H-imidazo[1,2-a]quinazoline-7-sulfonamide (22 mg, 44%). LCMS (ESI) m/z: 441.05 [M+H]⁺. ¹H NMR: (400 MHz, DMSO-d₆) δ 8.28 (d, J=2.1 Hz, 1H), 8.08 (s, 1H), 8.00 (dd, J=8.7, 2.2 Hz, 1H), 7.69 (s, 1H), 7.41 (s, 1H), 7.27 (d, J=8.6 Hz, 1H), 5.41-5.37 (m, 1H), 4.26 (dd, J=13.6, 10.2 Hz, 1H), 3.92 (dd, J=13.6, 5.0 Hz, 1H), 3.76-3.72 (m, 4H), 1.07 (s, 3H), 0.61-0.58 (m, 2H), 0.39-0.37 (m, 2H).

The following compounds in Table 10 were prepared using procedures similar to those described in Representative Synthesis Route 13 for Example 43 using appropriate starting

TABLE 10

| Example | | |
|---|---|---|
| No. | Name | [M + H]⁺ |
| 14 | (R)-1-(cyclopropylethynyl)-4-((1-methyl-1H-pyrazol-4- | 481.05 |
| | yl)methyl-d2)-N-(1-methylcyclopropyl)-5-oxo-1,2,4,5- | |
| | tetrahydroimidazo[1,2-a]quinazoline-7-sulfonamide | |
| 15 | (R)-9-bromo-1-(cyclopropylethynyl)-4-((1-methyl-1H- | 558.95, 560.85 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 16 | (R)-9-chloro-1-(cyclopropylethynyl)-4-((1-methyl-1H- | 515.00 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 49 | (R)-4-((1-methyl-1H-pyrazol-4-yl)methyl-d2)-N-(1- | 495.15 |
| | methylcyclopropyl)-1-((1-methylcyclopropyl)ethynyl)-5-oxo- | |
| | 1,2,4,5-tetrahydroimidazo[1,2-a]quinazoline-7-sulfonamide | |
| 55 | (R)-1-(3-(dimethylamino)prop-1-yn-1-yl)-4-((1-methyl-1H- | 498.10 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 92 | (S)-9-chloro-1-(cyclopropylethynyl)-4-((1-methyl-1H- | 515.15 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 95 | (S)-9-bromo-1-(cyclopropylethynyl)-4-((1-methyl-1H- | 558.95, 560.85 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 105 | (R)-1-(3-amino-3-methylbut-1-yn-1-yl)-4-((1-methyl-1H- | 498.10 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 106 | (7aS,10aR)-6-((1-methyl-1H-pyrazol-4-yl)methyl-d2)-N-(1- | 458.10 |
| | methylcyclopropyl)-5-oxo-6,7a,8,9,10,10a-hexahydro-5H- | |
| | pyrrolo[3′,4′:4,5]imidazo[1,2-a]quinazoline-3-sulfonamide | |
| 108 | (S)-4-((1-methyl-1H-pyrazol-4-yl)methyl-d2)-1-(((1-methyl- | 527.10 |
| | 1H-pyrazol-4-yl)oxy)methyl)-N-(1-methylcyclopropyl)-5- | |
| | oxo-1,2,4,5-tetrahydroimidazo[1,2-a]quinazoline-7- | |
| | sulfonamide | |
| 113 | (S)-4-((1-methyl-1H-pyrazol-4-yl)methyl-d2)-N-(1- | 477.20 |
| | methylcyclopropyl)-1-((methylthio)methyl)-5-oxo-1,2,4,5- | |
| | tetrahydroimidazo[1,2-a]quinazoline-7-sulfonamide | |
| 131 | 4-((1-methyl-1H-pyrazol-4-yl)methyl-d2)-N-(1- | 523.20 |
| | methylcyclopropyl)-5-oxo-1-((pyridin-2-ylamino)methyl)- | |
| | 1,2,4,5-tetrahydroimidazo[1,2-a]quinazoline-7-sulfonamide | |
| 274 | 8-fluoro-2,2-dimethyl-4-((1-methyl-1H-pyrazol-4-yl)methyl- | 463.00 |
| | d2)-N-(1-methylcyclopropyl)-5-oxo-1,2,4,5- | |
| | tetrahydroimidazo[1,2-a]quinazoline-7-sulfonamide | |
| 289 | (R)-1-ethynyl-8-fluoro-4-((1-methyl-1H-pyrazol-4-yl)methyl- | 459.15 |
| | d2)-N-(1-methylcyclopropyl)-5-oxo-1,2,4,5- | |
| | tetrahydroimidazo[1,2-a]quinazoline-7-sulfonamide | |

Representative Synthesis Route 14: Example 57: (R,E)-9-(4-(4-(4-methoxyphenyl)-4-oxobut-2-enoyl)piperazin-1-yl)-1,4-dimethyl-N-(1-methylcyclopropyl)-5-oxo-1,2,4,5-tetrahydroimidazo[1,2-a]quinazoline-7-sulfonamide

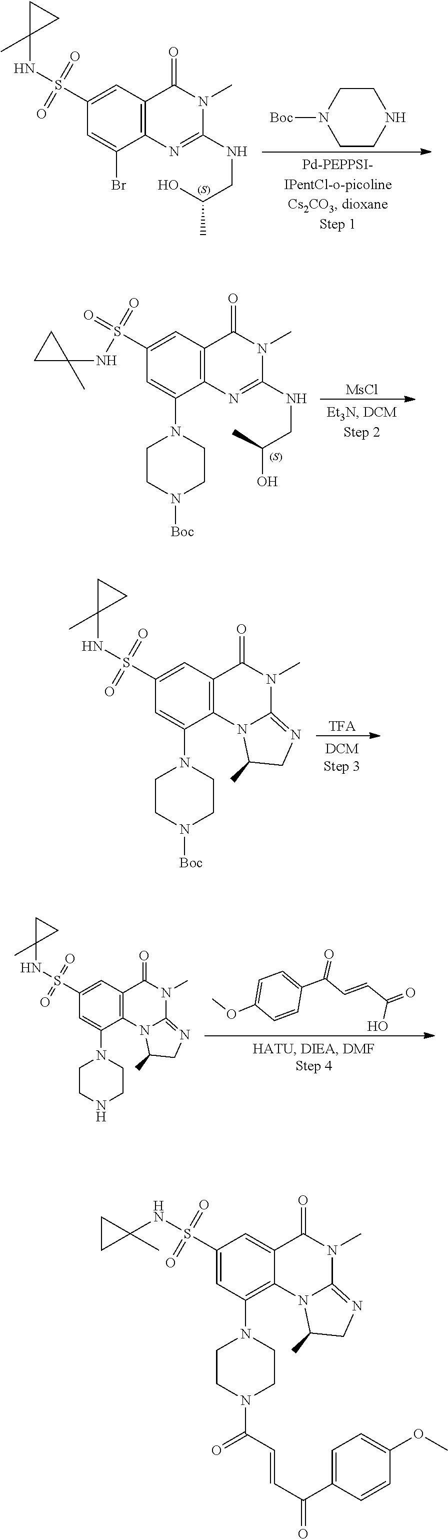

Step 1: To a stirred solution of 8-bromo-2-{[(2S)-2-hydroxypropyl]amino}-3-methyl-N-(1-methylcyclopropyl)-4-oxoquinazoline-6-sulfonamide (300 mg, 0.67 mmol) in anhydrous 1,4-dioxane (10 mL) was added tert-butyl piperazine-1-carboxylate (150.6 mg, 0.81 mmol) and Cs₂CO₃ (439 mg, 1.35 mmol) followed by catalytic amount of Pd-PEPPSI-IPentC1 2-methylpyridine-o-picoline (56.7 mg, 0.067 mmol) at room temperature. The resulting mixture was stirred for overnight at 100° C. under nitrogen atmosphere. The reaction mixture was purified by Prep-HPLC with the following conditions (Column: Xselect CSH F-Phenyl OBD column 30*250 mm, 5 m; Mobile Phase A: water (0.1% FA), Mobile Phase B: ACN; Flow rate: 60 mL/min mL/min; Gradient: 2% B to 20% B in 10 min; Wave Length: 254 nm/220 nm; RT1(min): 12.77; to afford tert-butyl 4-(2-{[(2S)-2-hydroxypropyl]amino}-3-methyl-6-[(1-methylcyclopropyl)sulfamoyl]-4-oxoquinazolin-8-yl)piperazine-1-carboxylate (250 mg) as a white solid.

Step 2: A solution of tert-butyl 4-(2-{[(2S)-2-hydroxypropyl]amino}-3-methyl-6-[(1-methylcyclopropyl)sulfamoyl]-4-oxoquinazolin-8-yl)piperazine-1-carboxylate (200 mg, 0.36 mmol) in DCM (10 mL) was treated with TEA (73.5 mg, 0.73 mmol) for 1 min at room temperature under nitrogen atmosphere followed by the addition of MsCl (83.2 mg, 0.73 mmol) in portions at room temperature. The reaction mixture was stirred at room temperature overnight. The resulting mixture was extracted with EtOAc (20 mL). The combined organic layers were washed with brine (20 mL), dried over anhydrous MgSO₄. After filtration, the filtrate was concentrated under reduced pressure. The residue was purified by reversed -phase flash chromatography with the following conditions: column, C18 silica gel; mobile phase, MeCN in water (0.1% FA), 10% to 50% gradient in 10 min; detector, UV 254 nm. This resulted in tert-butyl 4-[(1R)-1,4-dimethyl-7-[(1-methylcyclopropyl)sulfamoyl]-5-oxo-1H,2H-imidazo[1,2-a]quinazolin-9-yl]piperazine-1-carboxylate (150 mg) as a brown solid.

Step 3: A solution of tert-butyl 4-[(1R)-1,4-dimethyl-7-[(1-methylcyclopropyl)sulfamoyl]-5-oxo-1H,2H-imidazo[1,2-a]quinazolin-9-yl]piperazine-1-carboxylate (150 mg, 0.28 mmol) and TFA (64 mg, 0.56 mmol) in DCM (10 mL) was stirred for 30 min at room temperature under nitrogen atmosphere. The reaction was quenched with water at room temperature. The resulting mixture was concentrated under reduced pressure. This resulted in (1R)-1,4-dimethyl-N-(1-methylcyclopropyl)-5-oxo-9-(piperazin-1-yl)-1H,2H-imidazo[1,2-a]quinazoline-7-sulfonamide (100 mg) as a brown solid which was used in the next step directly without further purification.

Step 4: To a stirred solution of (1R)-1,4-dimethyl-N-(1-methylcyclopropyl)-5-oxo-9-(piperazin-1-yl)-1H,2H-imidazo[1,2-a]quinazoline-7-sulfonamide (30 mg, 0.069 mmol) in anhydrous DMF (5 mL) was added (2E)-4-(4-methoxyphenyl)-4-oxobut-2-enoic acid (17.2 mg, 0.083 mmol) and HATU (39.6 mg, 0.1 mmol) followed by DIEA (17.9 mg, 0.14 mmol) at 0° C. The reaction mixture was stirred at room temperature for 3 h. The reaction was quenched with water at room temperature. The resulting mixture was concentrated under reduced pressure. The crude product was purified by Prep-HPLC with the following conditions (Column: XBridge Shield RP18 OBD Column 30*150 mm, 5m; Mobile Phase A: water (10 mmol/L NH₄HCO₃+0.05% NH₃·H₂O), Mobile Phase B: MeOH; Flow rate: 60 mL/min mL/min; Gradient: 41% B to 61% B in 10 min; Wave Length: 254 nm/200 nm; RT1(min): 12.27) to afford (1R)-9-{4-[(2E)-4-(4-methoxyphenyl)-4-oxobut-2-enoyl]piperazin-1-yl}-1,4-dimethyl-N-(1-methylcyclopropyl)-5-oxo-1H,2H-imidazo[1,2-a]quinazoline-7-sulfonamide (5.8 mg, 14%). LCMS (ESI) m/z: 621.15 [M+H]⁺. ¹H NMR: (400 MHz, MeOH-d₄) δ 8.31 (d, J=2.0 Hz, 1H), 8.07 (t, J=8.8 Hz, 2H), 7.97-7.85 (m, 2H), 7.55 (dd, J=26, 15.2 Hz, 1H), 7.07 (dd, J=8.4, 5.6 Hz, 2H), 5.47 (q, J=6.8 Hz, 1H), 4.85-4.00 (m, 3H), 3.90 (d, J=4.0 Hz, 3H), 3.58-3.55 (m, 2H), 3.42 (d, J=1.2 Hz, 3H), 3.31-3.13 (4H), 2.48 (q, J=13.2 Hz, 1H), 1.19 (d, J=6.4 Hz, 3H), 1.13 (s, 3H), 0.71 (q, J=4.4 Hz, 2H), 0.49-0.36 (m, 2H).

The following compound in Table 11 was prepared using procedures similar to those described in Representative Synthesis Route 14 for Example 57 using appropriate starting materials and Pd(PEPPSI)Cl₂ in step 1.

TABLE 11

| Example | | |
|---|---|---|
| No. | Name | [M + H]⁺ |
| 125 | (R)-9-(4-(1-methoxycyclopropane-1-carbonyl)piperazin-1-yl)- | 531.15 |
| | 1,4-dimethyl-N-(1-methylcyclopropyl)-5-oxo-1,2,4,5- | |
| | tetrahydroimidazo[1,2-a]quinazoline-7-sulfonamide | |

Representative Synthesis Route 15: Example 73: (R)-1-methyl-4-((1-methyl-1H-pyrazol-4-yl)methyl)-N-(3-methyloxetan-3-yl)-5-oxo-1,2,4,5-tetrahydroimidazo[1,2-a]quinazoline-7-sulfonamide

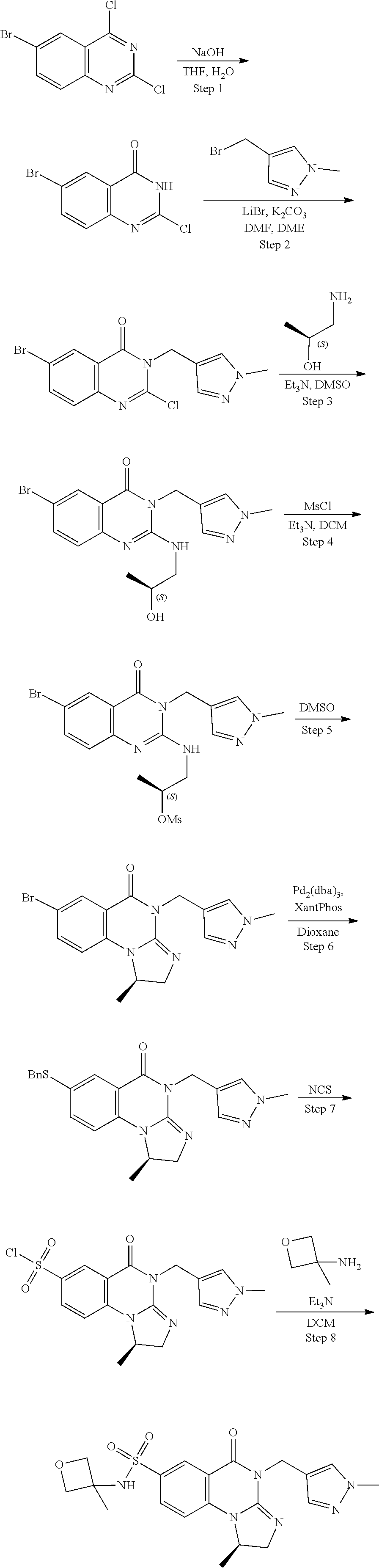

Step 1: A mixture of 6-bromo-2,4-dichloro-3,4-dihydroquinazoline (6 g, 21.4 mmol) and NaOH (1.71 g, 42.9 mmol) in THF (50 mL) was stirred for 2 h at 70° C. The reaction was quenched by the addition of water (300 mL) at room temperature. The resulting mixture was extracted with EtOAc (2×300 mL). The combined organic layers were washed with brine (300 mL), dried over anhydrous Na₂SO₄. After filtration, the filtrate was concentrated under reduced pressure. The residue was purified by silica gel column chromatography, eluted with EtOAc in PE (30% -40%) to afford 6-bromo-2-chloro-3H-quinazolin-4-one (3 g, 54%) as a yellow oil.

Step 2: To a stirred mixture of 6-bromo-2-chloro-3H-quinazolin-4-one (3 g, 11.6 mmol) in DME (24 mL) and DMF (6 mL) were added K₂CO₃ (6.39 g, 46.2 mmol), LiBr (1.00 g, 11.56 mmol) and 4-(bromomethyl)-1-methylpyrazole (2.02 g, 11.56 mmol) in portions at room temperature under nitrogen atmosphere. The resulting mixture was stirred for 2 h at room temperature. The reaction was quenched by the addition of water (200 mL) at room temperature. The resulting mixture was extracted with EtOAc (2×200 mL). The combined organic layers were washed with brine (200 mL), dried over anhydrous Na₂SO₄. After filtration, the filtrate was concentrated under reduced pressure. The residue was purified by silica gel column chromatography, eluted with EtOAc in PE (50%-70%) to afford 6-bromo-2-chloro-3-[(1-methylpyrazol-4-yl)methyl]quinazolin-4-one (2 g, 49%) as a yellow oil. LCMS (ESI) m/z: 353, 3 55 [M+H]⁺.

Step 3: To a stirred mixture of 6-bromo-2-chloro-3-[(1-methylpyrazol-4-yl)methyl]quinazolin-4-one (2 g, 5.66 mmol) and (2S)-1-aminopropan-2-ol (1.27 g, 16.9 mmol) in DMSO (20 mL) was added TEA (1.72 g, 16.9 mmol) dropwise at room temperature. The resulting mixture was stirred for 2 h at room temperature. The reaction was quenched by the addition of water (200 mL) at room temperature. The resulting mixture was extracted with EtOAc (2×200 mL). The combined organic layers were washed with brine (200 mL), dried over anhydrous Na₂SO₄. After filtration, the filtrate was concentrated under reduced pressure. The residue was purified by silica gel column chromatography, eluted with EtOAc in PE (50% -60%) to afford 6-bromo-2-{[(2S)-2-hydroxypropyl]amino}-3-[(1-methylpyrazol-4-yl)methyl]quinazolin-4-one (1.5 g, 68%) as a white oil. LCMS (ESI) m/z: 392, 394 [M+H]⁺.

Step 4: To a stirred mixture of 6-bromo-2-{[(2S)-2-hydroxypropyl]amino}-3-[(1-methylpyrazol-4-yl)methyl]quinazolin-4-one (1.5 g, 3.82 mmol) and MsCl (3.07 g, 26.8 mmol) in DCM (20 mL) was added TEA (1.93 g, 19.1 mmol) dropwise at room temperature. The resulting mixture was stirred for 1 h at room temperature under nitrogen atmosphere. The resulting mixture was concentrated under reduced pressure. The crude resulting mixture was used in the next step directly without further purification. LCMS (ESI) m/z: 470, 472 [M+H]⁺.

Step 5: A solution of (2S)-1-({6-bromo-3-[(1-methylpyrazol-4-yl)methyl]-4-oxoquinazolin-2-yl}amino)propan-2-yl methanesulfonate (500 mg, 1.06 mmol) in DMSO (10 mL) was stirred for 1 h at 40° C. The reaction was quenched by the addition of water (50 mL) at room temperature. The resulting mixture was extracted with EtOAc (2×50 mL). The combined organic layers were washed with brine (50 mL), dried over anhydrous Na₂SO₄. After filtration, the filtrate was concentrated under reduced pressure. The residue was purified by silica gel column chromatography, eluted with MeOH in DCM (10%-30%) to afford (1R)-7-bromo-1-methyl-4-[(1-methylpyrazol-4-yl)methyl]-1H,2H-imidazo[1,2-a]quinazolin-5-one (200 mg, 50%) as a yellow oil. LCMS (ESI) m/z: 374, 376 [M+H]⁺.

Step 6: To a stirred mixture of (1R)-7-bromo-1-methyl-4-[(1-methylpyrazol-4-yl)methyl]-1H,2H-imidazo[1,2-a]quinazolin-5-one (200 mg, 0.53 mmol) and benzyl mercaptan (53.1 mg, 0.43 mmol) in Dioxane (30 mL) were added Pd₂(dba)₃ (48.9 mg, 0.053 mmol), XantPhos (61.8 mg, 0.11 mmol) and DIEA (207.2 mg, 1.6 mmol) in portions at room temperature under nitrogen atmosphere. The resulting mixture was stirred for 2 h at 90° C. The reaction was quenched by the addition of water (50 mL) at room temperature. The resulting mixture was extracted with EtOAc (2×50 mL). The combined organic layers were washed with brine (50 mL), dried over anhydrous Na₂SO₄. After filtration, the filtrate was concentrated under reduced pressure. The residue was purified by silica gel column chromatography, eluted with MeOH in DCM (10%-35%) to afford (1R)-7-(benzylsulfanyl)-1-methyl-4-[(1-methylpyrazol-4-yl)methyl]-1H,2H-imidazo[1,2-a]quinazolin-5-one (120 mg, 54%) as a yellow oil. LCMS (ESI) m/z: 418 [M+H]⁺.

Step 7: A mixture of (1R)-7-(benzylsulfanyl)-1-methyl-4-[(1-methylpyrazol-4-yl)methyl]-1H,2H-imidazo[1,2-a]quinazolin-5-one (200 mg, 0.48 mmol) and NCS (304 mg, 1.92 mmol) in AcOH (5 mL) and H₂O (0.5 mL) was stirred for 1 h at room temperature. The resulting mixture was concentrated under reduced pressure. The crude resulting mixture was used in the next step directly without further purification.

Step 8: To a stirred mixture of (1R)-1-methyl-4-[(1-methylpyrazol-4-yl)methyl]-5-oxo-1H,2H-imidazo[1,2-a]quinazoline-7-sulfonyl chloride (70 mg, 0.18 mmol) and 3-methyloxetan-3 amine (107.2 mg, 1.25 mmol) in DCM (20 mL) was added TEA (125.9 mg, 1.25 mmol) dropwise at room temperature. The resulting mixture was stirred for 2 h at room temperature. The resulting mixture was concentrated under reduced pressure. The crude product was purified by Prep-HPLC with the following conditions (Column: XBridge Prep Phenyl OBD Column 19×250 mm, 5m; Mobile Phase A: water (10 mmol/L NH₄HCO₃+0.1% NH₃·H₂O), Mobile Phase B: ACN; Flow rate: 60 mL/min mL/min; Gradient: 12% B to 27% B in 10 min; Wave Length: 254 nm/220 nm; RT1(min): 12.503 to afford (1R)-1-methyl-N-(3-methyloxetan-3-yl)-4-[(1-methylpyrazol-4-yl)methyl]-5-oxo-1H,2H-imidazo[1,2-a]quinazoline-7-sulfonamide; formic acid (1.2 mg, 1%). LCMS (ESI) m/z: 445.10 [M+H]⁺. ¹H NMR: (400 MHz, DMSO-d₆) δ 8.38 (s, 1H), 8.26 (s, 1H), 7.96 (d, J=8.6 Hz, 1H), 7.68 (s, 1H), 7.42 (s, 1H), 7.29 (d, J=8.9 Hz, 1H), 4.99-4.81 (m, 2H), 4.81-4.70 (m, 1H), 4.58-4.48 (m, 2H), 4.17-4.08 (m, 2H), 4.08-4.06 (s, 1H), 3.80-3.74 (m, 3H) 1.40 (s, 3H), 1.33 (d, J=6.1 Hz, 3H).

Representative Synthesis Route 16: Example 84: (R)-3-methyl-9-(5-methyl-1,3,4-thiadiazol-2-yl)-N-(1-methylcyclopropyl)-2,9-dihydro-3H-benzo[d]imidazo[1,2-a]imidazole-7-sulfonamide

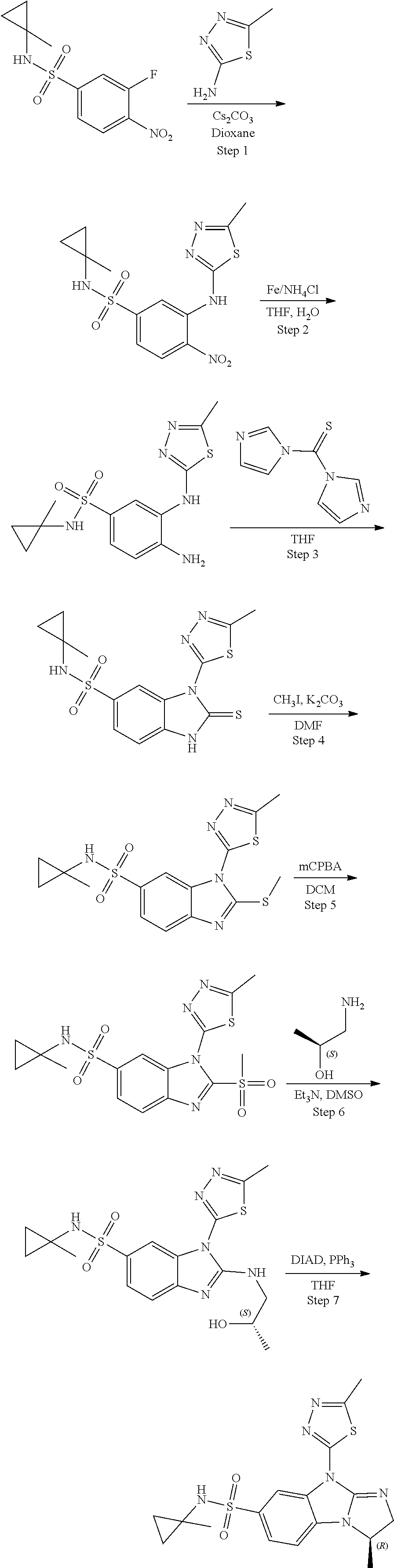

Step 1: To a stirred solution of 3-fluoro-N-(1-methylcyclopropyl)-4-nitrobenzenesulfonamide (5 g, 18.2 mmol) in anhydrous 1,4-dioxane (100 mL) was added was added Cs₂CO₃ (11.9 g, 36.5 mmol) followed by 5-methyl-1,3,4-thiadiazol-2-amine (2.10 g, 18.2 mmol) at 100° C. The reaction mixture was stirred at 100° C. for a period of 1 h. After completion of reaction, the reaction mixture was quenched by addition of water (50 mL). The aqueous layer was extracted with ethyl acetate (400 mL). The combined organic phase was washed with brine (100 mL), dried over anhydrous sodium sulfate and concentrated under reduced pressure to give crude product which was further purified by silica gel column chromatography (20-80% EtOAc/PE) to afford desired compound 3-[(5-methyl-1,3,4-thiadiazol-2-yl) amino]-N-(1-methylcyclopropyl)-4-nitrobenzenesulfonamide (2 g) as a yellow solid. LCMS (ESI) m/z: 370 [M+H]⁺.

Step 2: To a stirred solution of 3-[(5-methyl-1,3,4-thiadiazol-2-yl)amino]-N-(1-methylcyclopropyl)-4-nitrobenzenesulfonamide (2 g, 5.41 mmol) in anhydrous THF (80 mL) and H₂O (60 mL) was added NH₄Cl (2.9 g, 54.14 mmol) followed by Fe (1.21 g, 21.66 mmol) at room temperature. The reaction mixture was stirred at 70° C. for 1 h. After completion of reaction, the reaction mixture was quenched by addition of water (50 mL). The aqueous layer was extracted with ethyl acetate (400 mL). The combined organic phase was washed with brine (100 mL), dried over anhydrous sodium sulfate and concentrated under reduced pressure to give crude product which was further purified by column chromatography (20-80% EtOAc/PE) gradient to afford desired compound 4-amino-3-[(5-methyl-1,3,4-thiadiazol-2-yl)amino]-N-(1-methylcyclopropyl)benzenesulfonamide (1.2 g) as a brown solid. LCMS (ESI) m/z: 340 [M+H]⁺.

Step 3: To a stirred solution of 4-amino-3-[(5-methyl-1,3,4-thiadiazol-2-yl)amino]-N-(1-methylcyclopropyl)benzenesulfonamide (1.2 g, 3.53 mmol) in anhydrous THF (50 mL) was added 1-(imidazole-1-carbothioyl)imidazole (0.95 g, 5.3 mmol) at 0° C. The reaction mixture was stirred at room temperature overnight. After completion of reaction, the reaction mixture was quenched by addition of water (50 mL). The aqueous layer was extracted with ethyl acetate (400 mL). The combined organic phase was washed with brine (100 mL), dried over anhydrous sodium sulfate and concentrated under reduced pressure to give crude product which was further purified by column chromatography (0-10% MeOH/DCM) gradient to afford desired compound 3-(5-methyl-1,3,4-thiadiazol-2-yl)-N-(1-methylcyclopropyl)-2-sulfanylidene-1H-1,3-benzodiazole-5-sulfonamide (800 mg) as a brown solid. LCMS (ESI) m/z: 382 [M+H]⁺.

Step 4: To a stirred solution of 3-(5-methyl-1,3,4-thiadiazol-2-yl)-N-(1-methylcyclopropyl)-2-sulfanylidene-1H-1,3-benzodiazole-5-sulfonamide (800 mg, 2.09 mmol) in anhydrous DMF (20 mL) was added was added K₂CO₃ (579.6 mg, 4.19 mmol) followed by methyl iodide (208.4 mg, 1.47 mmol) at 0° C. The reaction mixture was stirred at 0° C. for 1 h. After completion of reaction, the reaction mixture was quenched by addition of water (30 mL). The aqueous layer was extracted with ethyl acetate (200 mL). The combined organic phase was washed with brine (100 mL), dried over anhydrous sodium sulfate and concentrated under reduced pressure to give crude product which was further purified by column chromatography (0-10% MeOH/DCM) to afford desired compound 3-(5-methyl-1,3,4-thiadiazol-2-yl)-N-(1-methylcyclopropyl)-2-(methylsulfanyl)-1,3-benzodiazole-5-sulfonamide (500 mg) as a yellow green solid. LCMS (ESI) m/z: 396 [M+H]⁺.

Step 5: To a stirred solution of 3-(5-methyl-1,3,4-thiadiazol-2-yl)-N-(1-methylcyclopropyl)-2-(methylsulfanyl)-1,3-benzodiazole-5-sulfonamide (500 mg, 1.26 mmol) in anhydrous DCM (20 mL) was added was added m-CPBA (436.3 mg, 2.53 mmol) at 0° C. The reaction mixture was stirred at 0° C. for 1 h. After completion of reaction, the reaction mixture was quenched by addition of Na₂SO₃ in water (50 mL). The aqueous layer was extracted with DCM (200 mL). The combined organic phase was washed with brine (100 mL), dried over anhydrous sodium sulfate and concentrated under reduced pressure to give crude product which was further purified by column chromatography (0-10% MeOH/DCM) to afford desired compound 2-methanesulfonyl-3-(5-methyl-1,3,4-thiadiazol-2-yl)-N-(1-methylcyclopropyl)-1,3-benzodiazole-5-sulfonamide (300 mg) as a yellow solid. LCMS (ESI) m/z: 428 [M+H]⁺.

Step 6: To a stirred solution of 2-methanesulfonyl-3-(5-methyl-1,3,4-thiadiazol-2-yl)-N-(1-methylcyclopropyl)-1,3-benzodiazole-5-sulfonamide (200 mg, 0.47 mmol) in anhydrous DCM (10 mL) was added TEA (142 mg, 1.4 mmol) followed by (2S)-1-aminopropan-2-ol (52.7 mg, 0.7 mmol) at room temperature. The reaction mixture was stirred at room temperature for 1 h. After completion of reaction, the reaction mixture was quenched by addition of water (10 mL). The aqueous layer was extracted with ethyl acetate (200 mL). The combined organic phase was washed with brine (100 mL), dried over anhydrous sodium sulfate and concentrated under reduced pressure to give crude product which was further purified by column chromatography (0-10% MeOH/DCM) to afford desired compound 2-{[(2S)-2-hydroxypropyl]amino}-3-(5-methyl-1,3,4-thiadiazol-2-yl)-N-(1-methylcyclopropyl)-1,3-benzodiazole-5-sulfonamide (120 mg) as a brown solid. LCMS (ESI) m/z: 423 [M+H]⁺.

Step 7: To a stirred solution of 2-{[(2S)-2-hydroxypropyl]amino}-3-(5-methyl-1,3,4-thiadiazol-2-yl)-N-(1-methylcyclopropyl)-1,3-benzodiazole-5-sulfonamide (50 mg, 0.12 mmol) in anhydrous DCM (3 mL) was added PPh₃ (62.1 mg, 0.24 mmol) and DIAD (47.9 mg, 0.24 mmol) at room temperature. The reaction mixture was stirred at room temperature for 2 h. After completion of reaction, the reaction mixture was quenched by addition of water (5 mL). The aqueous layer was extracted with ethyl acetate (20 mL). The combined organic phase was washed with brine (10 mL), dried over anhydrous sodium sulfate and concentrated under reduced pressure to give crude product which was further purified by column chromatography (0-10% MeOH/DCM) to afford desired compound (1.9 mg, 0.5%). LCMS (ESI) m/z: 404.95 [M+H]⁺. ¹H NMR: (400 MHz, CD₃CN) δ 8.86 (d, J=1.9 Hz, 1H), 7.79 (dd, J=8.4, 1.9 Hz, 1H), 7.65 (d, J=8.4 Hz, 1H), 6.11 (s, 1H), 3.10-3.01 (m, 1H), 2.89-2.85 (m, 1H), 2.76 (s, 3H), 2.52 (d, J=4.7 Hz, 1H), 1.38 (d, J=5.6 Hz, 3H), 1.09 (s, 3H), 0.69 (q, J=4.6 Hz, 2H), 0.42-0.38 (m, 2H).

The following compounds in Table 12 were prepared using procedures similar to those described in Representative Synthesis Route 16 for Example 84 using appropriate starting

TABLE 12

| Example | | |
|---|---|---|
| No. | Name | [M + H]⁺ |
| 85 | R)-10-(5-(difluoromethyl)-1,3,4-thiadiazol-2-yl)-4-methyl-N- | 455.10 |
| | (1-methylcyclopropyl)-2,3,4,10- | |
| | tetrahydrobenzo[4,5]imidazo[1,2-a]pyrimidine-8-sulfonamide | |
| 86 | 10-(5-(difluoromethyl)-1,3,4-thiadiazol-2-yl)-N-(1- | 442.90 |
| | methylcyclopropyl)-3,4-dihydro-10H-benzo[4,5]imidazo[2,1- | |
| | c][1,2,4]oxadiazine-8-sulfonamide | |

Representative Synthesis Route 17: Example 99: (R)-1-(3,3-difluoroprop-1-yn-1-yl)-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

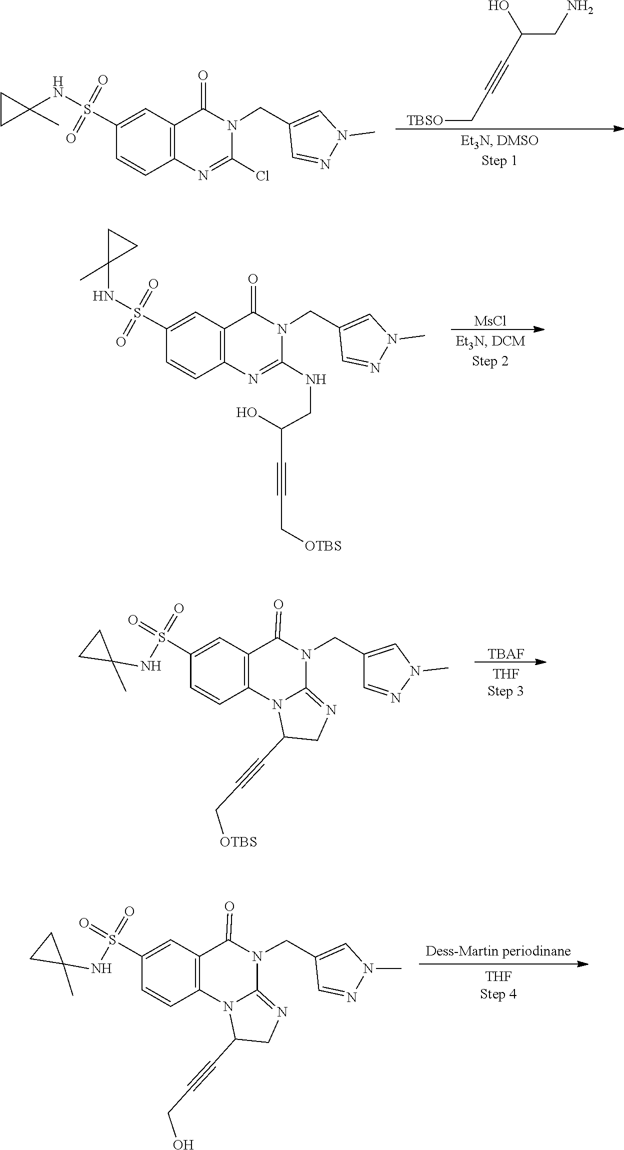

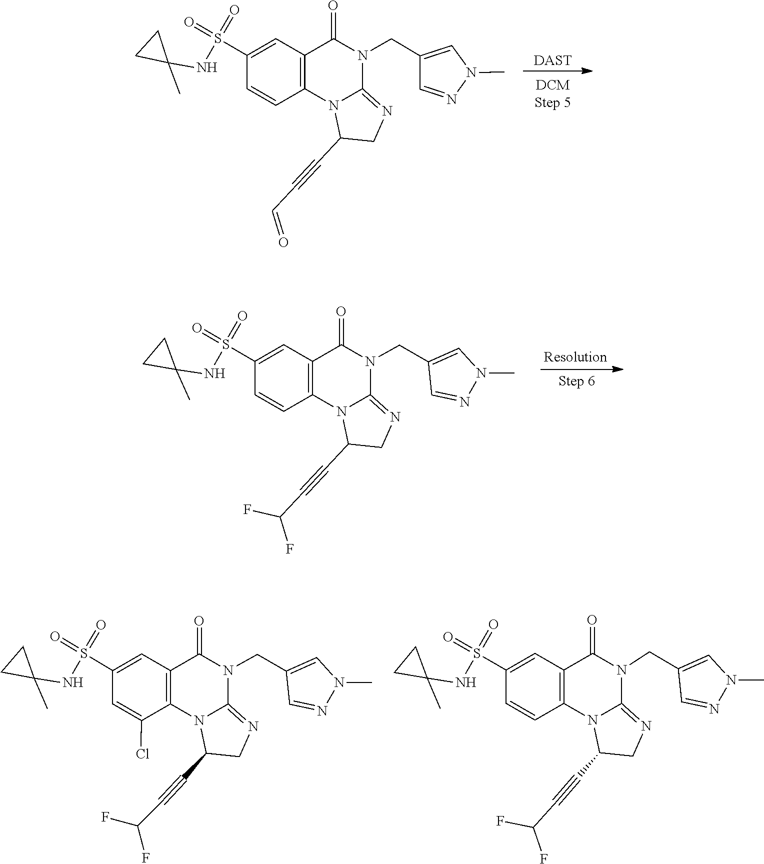

Step 1: To a stirred solution of 2-chloro-N-(1-methylcyclopropyl)-3-[(1-methylpyrazol-4-yl)methyl]-4-oxoquinazoline-6-sulfonamide (300 mg, 0.74 mmol) and Et₃N (223.3 mg, 2.2 mmol) in DMSO (10 mL) was added 1-amino-5-[(tert-butyldimethylsilyl)oxy]pent-3-yn-2-ol (337.4 mg, 1.47 mmol) at room temperature. The resulting mixture was stirred at room temperature for 2 h. The reaction was quenched by the addition of water (100 mL) at room temperature. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with CH₂Cl₂/MeOH (0%-10%) to afford 2-({5-[(tert-butyldimethylsilyl)oxy]-2-hydroxypent-3-yn-1-yl}amino)-N-(1-methylcyclopropyl)-3-[(1-methylpyrazol-4-yl)methyl]-4-oxoquinazoline-6-sulfonamide (260 mg, 59%) as a yellow solid. LCMS (ESI) m/z: 601.0 [M+H]⁺.

Step 2: To a stirred solution of 2-({5-[(tert-butyldimethylsilyl)oxy]-2-hydroxypent-3-yn-1-yl}amino)-N-(1-methylcyclopropyl)-3-[(1-methylpyrazol-4-yl)methyl]-4-oxoquinazoline-6-sulfonamide (250 mg, 0.41 mmol) and MsCl (238.3 mg, 2.08 mmol) in DCM (10 mL) was added Et₃N (421 mg, 4.16 mmol) at room temperature. The resulting mixture was stirred at room temperature for 16 h. The reaction was quenched by the addition of water (50 mL) at room temperature. The resulting mixture was extracted with CH₂Cl₂ (3×30 mL). The combined organic layers were washed with brine (2×30 mL), dried over anhydrous Na₂SO₄.

After filtration, the filtrate was concentrated under reduced pressure. The residue was purified by silica gel column chromatography, eluted with CH₂Cl₂/MeOH (5%-15%) to afford 1-{3-[(tert-butyldimethylsilyl)oxy]prop-1-yn-1-yl}-N-(1-methylcyclopropyl)-4-[(1-methylpyrazol-4-yl)methyl]-5-oxo-1H,2H-imidazo[1,2-a]quinazoline-7-sulfonamide (230 mg, 95%) as a yellow solid. LCMS (ESI) m/z: 583.0 [M+H]⁺.

Step 3: A mixture of 1-{3-[(tert-butyldimethylsilyl)oxy]prop-1-yn-1-yl}-N-(1-methylcyclopropyl)-4-[(1-methylpyrazol-4-yl)methyl]-5-oxo-1H,2H-imidazo[1,2-a]quinazoline-7-sulfonamide (230 mg, 0.39 mmol) and TBAF (103.2 mg, 0.39 mmol) in THF (10 mL) was stirred for 2 h at room temperature. The reaction was quenched by the addition of water (100 mL) at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure to afford 1-(3-hydroxyprop-1-yn-1-yl)-N-(1-methylcyclopropyl)-4-[(1-methylpyrazol-4-yl)methyl]-5-oxo-1H,2H-imidazo[1,2-a]quinazoline-7-sulfonamide (180 mg, 97%) as a yellow solid. LCMS (ESI) m/z: 469.0 [M+H]⁺.

Step 4: A mixture of 1-(3-hydroxyprop-1-yn-1-yl)-N-(1-methylcyclopropyl)-4-[(1-methylpyrazol-4-yl)methyl]-5-oxo-1H,2H-imidazo[1,2-a]quinazoline-7-sulfonamide (110 mg, 0.23 mmol,) and Dess-Martin reagent (99.6 mg, 0.23 mmol) in THF (10 mL) was stirred for 2 h at 0° C. The reaction was quenched by the addition of water (30 mL) at room temperature. The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by silica gel column chromatography, eluted with CH₂Cl₂/MeOH (5%-15%) to afford N-(1-methylcyclopropyl)-4-[(1-methylpyrazol-4-yl)methyl]-5-oxo-1-(3-oxoprop-1-yn-1-yl)-1H,2H-imidazo[1,2-a]quinazoline-7-sulfonamide (40 mg, 36%) as a yellow solid. LCMS (ESI) m/z: 467.0 [M+H]⁺.

Step 5: A solution of N-(1-methylcyclopropyl)-4-[(1-methylpyrazol-4-yl)methyl]-5-oxo-1-(3-oxoprop-1-yn-1-yl)-1H,2H-imidazo[1,2-a]quinazoline-7-sulfonamide (40 mg, 0.08 mmol) and DAST (27.5 mg, 0.17 mmol) in DCM (10 mL) was stirred for 1 h at 0° C. The reaction was quenched by the addition of water (30 mL) at room temperature. The resulting mixture was extracted with CH₂Cl₂ (3×10 mL). The combined organic layers were washed with water (2×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 30% to 50% gradient in 30 min; detector, UV 254 nm. The resulting mixture was dried under vacuum to afford 1-(3,3-difluoroprop-1-yn-1-yl)-N-(1-methylcyclopropyl)-4-[(1-methylpyrazol-4-yl)methyl]-5-oxo-1H,2H-imidazo[1,2-a]quinazoline-7-sulfonamide (25 mg, 60%) as a yellow solid. LCMS (ESI) m/z: 489.0 [M+H]⁺.

Step 6: The racemate (20 mg) was purified by chiral-Prep-HPLC with the following conditions (Mobile Phase A: Hex (0.1% DEA): EtOH=50:50; Flow rate: 1.67 ml/min mL/min; Gradient: isocratic) to afford (1R)-1-(3,3-difluoroprop-1-yn-1-yl)-N-(1-methylcyclopropyl)-4-[(1-methylpyrazol-4-yl)methyl]-5-oxo-1H,2H-imidazo[1,2-a]quinazoline-7-sulfonamide (3.0 mg, 16%) and (1 S)-1-(3,3-difluoroprop-1-yn-1-yl)-N-(1-methylcyclopropyl)-4-[(1-methylpyrazol-4-yl)methyl]-5-oxo-1H,2H-imidazo[1,2-a]quinazoline-7-sulfonamide (3.2 mg, 17%) both as white solids. LCMS (ESI) m/z: 489.00 [M+H]⁺. ¹H NMR (400 MHz, Methanol-d₄) δ 8.49-8.42 (m, 1H), 8.09-8.01 (m, 1H), 7.69 (s, 1H), 7.56 (s, 1H), 7.27 (dd, J=8.8, 2.4 Hz, 1H), 6.60-6.24 (m, 1H), 5.49 (dt, J=8.8, 4.4 Hz, 1H), 5.09-4.96 (m, 2H), 4.38 (dd, J=13.6, 10.4 Hz, 1H), 4.11 (dd, J=13.6, 4.8 Hz, 1H), 3.82 (s, 3H), 1.15 (s, 3H), 0.70 (q, J=4.8 Hz, 2H), 0.51-0.39 (m, 2H).

Representative Synthesis Route 18: Example 101: (R)-1-((3-fluoro-1-methyl-1H-pyrazol-4-yl)ethynyl)-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide

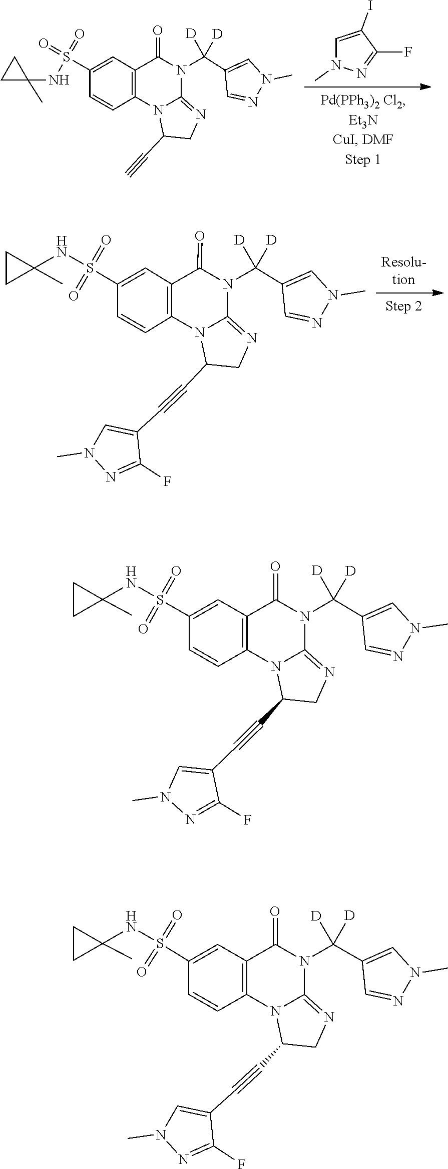

Step 1: A solution of 1-ethynyl-N-(1-methylcyclopropyl)-4-[(1-methylpyrazol-4-yl)(2H2)methyl]-5-oxo-1H,2H-imidazo[1,2-a]quinazoline-7-sulfonamide (120 mg, 0.27 mmol), 3-fluoro-4-iodo-1-methylpyrazole (73.8 mg, 0.33 mmol), Et₃N (82.7 mg, 0.82 mmol), CuI (5.19 mg, 0.027 mmol) and Pd(PPh₃)₂Cl₂ (19.1 mg, 0.027 mmol) in DMF (2 mL) was stirred for 2 h at 60° C. under nitrogen atmosphere. The residue was purified by reversed-phase flash chromatography with the following conditions: column, C18 silica gel; mobile phase, MeCN in water (0.1% NH₃·H₂O), 20% to 50% gradient in 20 min). This resulted in 1-[2-(3-fluoro-1-methylpyrazol-4-yl)ethynyl]-N-(1-methylcyclopropyl)-4-[(1-methylpyrazol-4-yl)(2H2)methyl]-5-oxo-1H,2H-imidazo[1,2-a]quinazoline-7-sulfonamide (80 mg, 54%) as a yellow solid. LCMS (ESI) m/z: 539 [M+H]⁺.

Step 2: The racemate (80 mg) was purified by Chiral-HPLC with the following conditions (Column: XA-CHIRAL ART Cellulose-SZ, 3*25 cm Sum; Mobile Phase A: Hex (10 mM NH₃-MeOH), Mobile Phase B: EtOH; Flow rate: 40 mL/min; Gradient: isocratic 50; Wave Length: 210/200 nm; Sample Solvent: MeOH: DCM=1:1) to afford (1R)-1-[2-(3-fluoro-1-methylpyrazol-4-yl)ethynyl]-N-(1-methylcyclopropyl)-4-[(1-methylpyrazol-4-yl)(2H2)methyl]-5-oxo-1H,2H-imidazo[1,2-a]quinazoline-7-sulfonamide (27.6 mg, 34%), (1 S)-1-[2-(3-fluoro-1-methylpyrazol-4-yl)ethynyl]-N-(1-methylcyclopropyl)-4-[(1-methylpyrazol-4-yl)(2H2)methyl]-5-oxo-1H,2H-imidazo[1,2-a]quinazoline-7-sulfonamide (29.6 mg, 36%) both as white solids. LCMS (ESI) m/z: 539.05 [M+H]⁺. 1H NMR (400 MHz, DMSO-d₆) δ 8.28 (d, J=2.2 Hz, 1H), 8.07 (s, 1H), 8.00 (dd, J=8.8, 2.2 Hz, 1H), 7.95 (d, J=2.0 Hz, 1H), 7.70 (s, 1H), 7.42 (s, 1H), 7.32 (d, J=8.7 Hz, 1H), 5.62 (dd, J=10.2, 5.2 Hz, 1H), 4.32 (dd, J=13.6, 10.2 Hz, 1H), 3.97 (dd, J=13.6, 5.2 Hz, 1H), 3.77 (s, 3H), 3.69 (s, 3H), 1.06 (s, 3H), 0.62-0.56 (m, 2H), 0.40-0.35 (m, 2H).

The following compounds in Table 13 were prepared using procedures similar to those described in Representative Synthesis Route 18 for Example 101 using appropriate starting

TABLE 13

| Example | | |
|---|---|---|
| No. | Name | [M + H]⁺ |
| 97 | (R)-1-((1-methyl-1H-pyrazol-4-yl)ethynyl)-4-((1-methyl-1H- | 521.10 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 104 | (R)-1-((6-aminopyridin-2-yl)ethynyl)-4-((1-methyl-1H- | 533.10 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 116 | (R)-4-((1-methyl-1H-pyrazol-4-yl)methyl)-N-(1- | 516.05 |
| | methylcyclopropyl)-5-oxo-1-(pyridin-4-ylethynyl)-1,2,4,5- | |
| | tetrahydroimidazo[1,2-a]quinazoline-7-sulfonamide | |
| 117 | (R)-4-((1-methyl-1H-pyrazol-4-yl)methyl)-N-(1- | 516.10 |
| | methylcyclopropyl)-5-oxo-1-(pyridin-3-ylethynyl)-1,2,4,5- | |
| | tetrahydroimidazo[1,2-a]quinazoline-7-sulfonamide | |
| 120 | (R)-1-((3-fluoro-1-methyl-1H-pyrazol-4-yl)ethynyl)-4-methyl- | 457.00 |
| | N-(1-methylcyclopropyl)-5-oxo-1,2,4,5- | |
| | tetrahydroimidazo[1,2-a]quinazoline-7-sulfonamide | |
| 165 | (R)-4-((1-methyl-1H-pyrazol-4-yl)methyl)-1-((1-methyl-1H- | 519.10 |
| | pyrazol-5-yl)ethynyl)-N-(1-methylcyclopropyl)-5-oxo-1,2,4,5- | |
| | tetrahydroimidazo[1,2-a]quinazoline-7-sulfonamide | |
| 166 | (R)-1-((1-methyl-1H-1,2,3-triazol-5-yl)ethynyl)-4-((1-methyl- | 520.10 |
| | 1H-pyrazol-4-yl)methyl)-N-(1-methylcyclopropyl)-5-oxo- | |
| | 1,2,4,5-tetrahydroimidazo[1,2-a]quinazoline-7-sulfonamide | |
| 167 | 1-(imidazo[1,2-a]pyridin-3-ylethynyl)-4-methyl-N-(1- | 475.10 |
| | methylcyclopropyl)-5-oxo-1,2,4,5-tetrahydroimidazo[1,2- | |
| | a]quinazoline-7-sulfonamide | |
| 168 | 1-((1H-pyrrolo[2,3-c]pyridin-3-yl)ethynyl)-4-methyl-N-(1- | 475.10 |
| | methylcyclopropyl)-5-oxo-1,2,4,5-tetrahydroimidazo[1,2- | |
| | a]quinazoline-7-sulfonamide | |
| 169 | 1-((1H-pyrrolo[2,3-b]pyridin-2-yl)ethynyl)-4-methyl-N-(1- | 475.20 |
| | methylcyclopropyl)-5-oxo-1,2,4,5-tetrahydroimidazo[1,2- | |
| | a]quinazoline-7-sulfonamide | |
| 170 | 1-((2,3-dihydro-[1,4]dioxino[2,3-b]pyridin-8-yl)ethynyl)-4- | 494.20 |
| | methyl-N-(1-methylcyclopropyl)-5-oxo-1,2,4,5- | |
| | tetrahydroimidazo[1,2-a]quinazoline-7-sulfonamide | |
| 171 | (R)-1-(imidazo[1,2-a]pyrimidin-3-ylethynyl)-4-methyl-N-(1- | 476.10 |
| | methylcyclopropyl)-5-oxo-1,2,4,5-tetrahydroimidazo[1,2- | |
| | a]quinazoline-7-sulfonamide | |
| 174 | (R)-4-methyl-1-((2-methyl-2H-indazol-3-yl)ethynyl)-N-(1- | 489.20 |
| | methylcyclopropyl)-5-oxo-1,2,4,5-tetrahydroimidazo[1,2- | |
| | a]quinazoline-7-sulfonamide | |
| 176 | 1-(imidazo[1,2-b]pyridazin-6-ylethynyl)-4-methyl-N-(1- | 476.20 |
| | methylcyclopropyl)-5-oxo-1,2,4,5-tetrahydroimidazo[1,2- | |
| | a]quinazoline-7-sulfonamide | |
| 178 | 1-((5′H,7′H-spiro[oxetane-3,6′-pyrazolo[5,1-b][1,3]oxazin]-3′- | 523.10 |
| | yl)ethynyl)-4-methyl-N-(1-methylcyclopropyl)-5-oxo-1,2,4,5- | |
| | tetrahydroimidazo[1,2-a]quinazoline-7-sulfonamide | |
| 180 | (R)-1-((6-cyanoimidazo[1,2-b]pyridazin-3-yl)ethynyl)-4- | 501.10 |
| | methyl-N-(1-methylcyclopropyl)-5-oxo-1,2,4,5- | |
| | tetrahydroimidazo[1,2-a]quinazoline-7-sulfonamide | |
| 181 | (R)-1-((6-methoxyimidazo[1,2-b]pyridazin-3-yl)ethynyl)-4- | 506.20 |
| | methyl-N-(1-methylcyclopropyl)-5-oxo-1,2,4,5- | |
| | tetrahydroimidazo[1,2-a]quinazoline-7-sulfonamide | |
| 182 | (R)-4-methyl-N-(1-methylcyclopropyl)-1-((7- | 490.10 |
| | methylimidazo[1,2-b]pyridazin-3-yl)ethynyl)-5-oxo-1,2,4,5- | |
| | tetrahydroimidazo[1,2-a]quinazoline-7-sulfonamide | |
| 183 | (R)-4-methyl-N-(1-methylcyclopropyl)-1-((6- | 490.10 |
| | methylimidazo[1,2-b]pyridazin-3-yl)ethynyl)-5-oxo-1,2,4,5- | |
| | tetrahydroimidazo[1,2-a]quinazoline-7-sulfonamide | |
| 184 | 1-(imidazo[1,2-b]pyridazin-3-ylethynyl)-4-(methyl-d3)-N-(1- | 479.10 |
| | methylcyclopropyl)-5-oxo-1,2,4,5-tetrahydroimidazo[1,2- | |
| | a]quinazoline-7-sulfonamide | |
| 185 | (R)-4-methyl-1-((1-methyl-1H-pyrazol-5-yl)ethynyl)-N-(1- | 439.10 |
| | methylcyclopropyl)-5-oxo-1,2,4,5-tetrahydroimidazo[1,2- | |
| | a]quinazoline-7-sulfonamide | |
| 190 | (R)-4-ethyl-N-(1-methylcyclopropyl)-5-oxo-1-(pyrazolo[1,5- | 490.10 |
| | a]pyrimidin-3-ylethynyl)-1,2,4,5-tetrahydroimidazo[1,2- | |
| | a]quinazoline-7-sulfonamide | |
| 191 | (R)-1-((7-((dimethylamino)methyl)imidazo[1,2-b]pyridazin-3- | 547.15 |
| | yl)ethynyl)-4-ethyl-N-(1-methylcyclopropyl)-5-oxo-1,2,4,5- | |
| | tetrahydroimidazo[1,2-a]quinazoline-7-sulfonamide | |
| 192 | (R)-1-((1,3-dimethyl-1H-pyrazol-5-yl)ethynyl)-4-((1-methyl- | 533.20 |
| | 1H-pyrazol-4-yl)methyl)-N-(1-methylcyclopropyl)-5-oxo- | |
| | 1,2,4,5-tetrahydroimidazo[1,2-a]quinazoline-7-sulfonamide | |
| 194 | (R)-4-((1-methyl-1H-pyrazol-4-yl)methyl)-N-(1- | 536.00 |
| | methylcyclopropyl)-1-((3-methylisothiazol-5-yl)ethynyl)-5- | |
| | oxo-1,2,4,5-tetrahydroimidazo[1,2-a]quinazoline-7- | |
| | sulfonamide | |
| 197 | (R)-4-((1-methyl-1H-pyrazol-4-yl)methyl-d2)-1-((1-methyl- | 521.10 |
| | 1H-pyrazol-5-yl)ethynyl)-N-(1-methylcyclopropyl)-5-oxo- | |
| | 1,2,4,5-tetrahydroimidazo[1,2-a]quinazoline-7-sulfonamide | |
| 200 | 4-methyl-1-((1-methyl-2-oxo-1,2-dihydropyridin-4- | 466.10 |
| | yl)ethynyl)-N-(1-methylcyclopropyl)-5-oxo-1,2,4,5- | |
| | tetrahydroimidazo[1,2-a]quinazoline-7-sulfonamide | |
| 201 | (R)-4-methyl-N-(1-methylcyclopropyl)-5-oxo-1-((6- | 544.20 |
| | (trifluoromethyl)imidazo[1,2-b]pyridazin-3-yl)ethynyl)- | |
| | 1,2,4,5-tetrahydroimidazo[1,2-a]quinazoline-7-sulfonamide | |
| 202 | 1-((6-cyanopyridin-3-yl)ethynyl)-4-methyl-N-(1- | 461.10 |
| | methylcyclopropyl)-5-oxo-1,2,4,5-tetrahydroimidazo[1,2- | |
| | a]quinazoline-7-sulfonamide | |
| 203 | 4-methyl-N-(1-methylcyclopropyl)-1-((6-methylpyridin-3- | 450.10 |
| | yl)ethynyl)-5-oxo-1,2,4,5-tetrahydroimidazo[1,2- | |
| | a]quinazoline-7-sulfonamide | |
| 204 | 1-((6-methoxypyridin-3-yl)ethynyl)-4-methyl-N-(1- | 466.10 |
| | methylcyclopropyl)-5-oxo-1,2,4,5-tetrahydroimidazo[1,2- | |
| | a]quinazoline-7-sulfonamide | |
| 209 | 4-methyl-N-(1-methylcyclopropyl)-5-oxo-1-((7- | 544.10 |
| | (trifluoromethyl)imidazo[1,2-b]pyridazin-3-yl)ethynyl)- | |
| | 1,2,4,5-tetrahydroimidazo[1,2-a]quinazoline-7-sulfonamide | |
| 210 | (R)-1-((1-(difluoromethyl)-1H-pyrazol-5-yl)ethynyl)-4-((1- | 555.25 |
| | methyl-1H-pyrazol-4-yl)methyl)-N-(1-methylcyclopropyl)-5- | |
| | oxo-1,2,4,5-tetrahydroimidazo[1,2-a]quinazoline-7- | |
| | sulfonamide | |
| 215 | 8-fluoro-1-(imidazo[1,2-b]pyridazin-3-ylethynyl)-4-methyl-N- | 494.00 |
| | (1-methylcyclopropyl)-5-oxo-1,2,4,5-tetrahydroimidazo[1,2- | |
| | a]quinazoline-7-sulfonamide | |
| 216 | (R)-4-methyl-N-(1-methylcyclopropyl)-1-((2-methylpyridin-4- | 450.10 |
| | yl)ethynyl)-5-oxo-1,2,4,5-tetrahydroimidazo[1,2- | |
| | a]quinazoline-7-sulfonamide | |
| 224 | 1-((2-fluoropyridin-4-yl)ethynyl)-4-methyl-N-(1- | 454.00 |
| | methylcyclopropyl)-5-oxo-1,2,4,5-tetrahydroimidazo[1,2- | |
| | a]quinazoline-7-sulfonamide | |
| 225 | 4-methyl-N-(1-methylcyclopropyl)-1-((6-methylpyridin-2- | 450.00 |
| | yl)ethynyl)-5-oxo-1,2,4,5-tetrahydroimidazo[1,2- | |
| | a]quinazoline-7-sulfonamide | |
| 226 | 4-methyl-N-(1-methylcyclopropyl)-5-oxo-1-((2- | 504.00 |
| | (trifluoromethyl)pyridin-4-yl)ethynyl)-1,2,4,5- | |
| | tetrahydroimidazo[1,2-a]quinazoline-7-sulfonamide | |
| 227 | 1-((2-methoxypyridin-4-yl)ethynyl)-4-methyl-N-(1- | 466.00 |
| | methylcyclopropyl)-5-oxo-1,2,4,5-tetrahydroimidazo[1,2- | |
| | a]quinazoline-7-sulfonamide | |
| 238 | (R)-4-((1-methyl-1H-pyrazol-4-yl)methyl)-N-(1- | 530.15 |
| | methylcyclopropyl)-1-((2-methylpyridin-4-yl)ethynyl)-5-oxo- | |
| | 1,2,4,5-tetrahydroimidazo[1,2-a]quinazoline-7-sulfonamide | |
| 242 | (R)-4-((1-methyl-1H-pyrazol-4-yl)methyl-d2)-N-(1- | 532.00 |
| | methylcyclopropyl)-1-((3-methylpyridin-2-yl)ethynyl)-5-oxo- | |
| | 1,2,4,5-tetrahydroimidazo[1,2-a]quinazoline-7-sulfonamide | |
| 245 | (R)-4-ethyl-1-((4-fluoro-1-methyl-1H-pyrazol-5-yl)ethynyl)- | 471.20 |
| | N-(1-methylcyclopropyl)-5-oxo-1,2,4,5- | |
| | tetrahydroimidazo[1,2-a]quinazoline-7-sulfonamide | |
| 246 | (R)-4-((1-methyl-1H-pyrazol-4-yl)methyl-d2)-N-(1- | 519.20 |
| | methylcyclopropyl)-5-oxo-1-(pyrimidin-5-ylethynyl)-1,2,4,5- | |
| | tetrahydroimidazo[1,2-a]quinazoline-7-sulfonamide | |
| 249 | (R)-1-((3-methoxypyrazin-2-yl)ethynyl)-4-((1-methyl-1H- | 549.20 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 250 | (R)-1-((2-aminopyridin-3-yl)ethynyl)-4-((1-methyl-1H- | 533.30 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 251 | (R)-1-((3,5-dimethylpyrazin-2-yl)ethynyl)-4-((1-methyl-1H- | 547.20 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 252 | (R)-1-((5-aminopyridin-3-yl)ethynyl)-4-((1-methyl-1H- | 533.20 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 253 | (R)-1-((5-(dimethylamino)pyridin-3-yl)ethynyl)-4-((1-methyl- | 561.20 |
| | 1H-pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 258 | (R)-1-((2-aminopyrimidin-5-yl)ethynyl)-4-((1-methyl-1H- | 534.20 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 259 | (R)-1-((2-methoxypyrimidin-5-yl)ethynyl)-4-((1-methyl-1H- | 549.20 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 260 | (R)-4-((1-methyl-1H-pyrazol-4-yl)methyl-d2)-N-(1- | 519.20 |
| | methylcyclopropyl)-5-oxo-1-(pyridazin-3-ylethynyl)-1,2,4,5- | |
| | tetrahydroimidazo[1,2-a]quinazoline-7-sulfonamide | |
| 261 | (R)-1-((2-cyclopropylpyrimidin-5-yl)ethynyl)-4-((1-methyl- | 559.20 |
| | 1H-pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 262 | (R)-1-((4-methoxypyrimidin-5-yl)ethynyl)-4-((1-methyl-1H- | 549.20 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 263 | (R)-4-((1-methyl-1H-pyrazol-4-yl)methyl-d2)-1-((4- | 548.20 |
| | (methylamino)pyrimidin-5-yl)ethynyl)-N-(1- | |
| | methylcyclopropyl)-5-oxo-1,2,4,5-tetrahydroimidazo[1,2- | |
| | a]quinazoline-7-sulfonamide | |
| 268 | (R)-1-((4-aminopyrimidin-5-yl)ethynyl)-4-((1-methyl-1H- | 534.10 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 269 | (R)-1-((5-methoxypyridin-3-yl)ethynyl)-4-((1-methyl-1H- | 548.20 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 275 | (R)-4-((1-methyl-1H-pyrazol-4-yl)methyl-d2)-N-(1- | 533.20 |
| | methylcyclopropyl)-1-((2-methylpyrimidin-5-yl)ethynyl)-5- | |
| | oxo-1,2,4,5-tetrahydroimidazo[1,2-a]quinazoline-7- | |
| | sulfonamide | |
| 277 | (R)-4-(cyclopropylmethyl)-1-((1,3-dimethyl-1H-pyrazol-4- | 493.25 |
| | yl)ethynyl)-N-(1-methylcyclopropyl)-5-oxo-1,2,4,5- | |
| | tetrahydroimidazo[1,2-a]quinazoline-7-sulfonamide | |
| 280 | (R)-1-((6-(dimethylamino)pyridin-3-yl)ethynyl)-4-((1-methyl- | 561.00 |
| | 1H-pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 281 | (R)-4-((1-methyl-1H-pyrazol-4-yl)methyl-d2)-N-(1- | 522.30 |
| | methylcyclopropyl)-5-oxo-1-((1,2,3,6-tetrahydropyridin-4- | |
| | yl)ethynyl)-1,2,4,5-tetrahydroimidazo[1,2-a]quinazoline-7- | |
| | sulfonamide | |
| 282 | (R)-1-((3,6-dihydro-2H-pyran-4-yl)ethynyl)-4-((1-methyl-1H- | 523.20 |
| | pyrazol-4-yl)methyl-d2)-N-(1-methylcyclopropyl)-5-oxo- | |
| | 1,2,4,5-tetrahydroimidazo[1,2-a]quinazoline-7-sulfonamide | |

Representative Synthesis Route 19: Example 102: (R)-1-methyl-N-(1-methylcyclopropyl)-5-oxo-4-((1-(prop-2-yn-1-yl)-1H-pyrazol-4-yl)methyl)-1,2,4,5-tetrahydroimidazo[1,2-a]quinazoline-7-sulfonamide

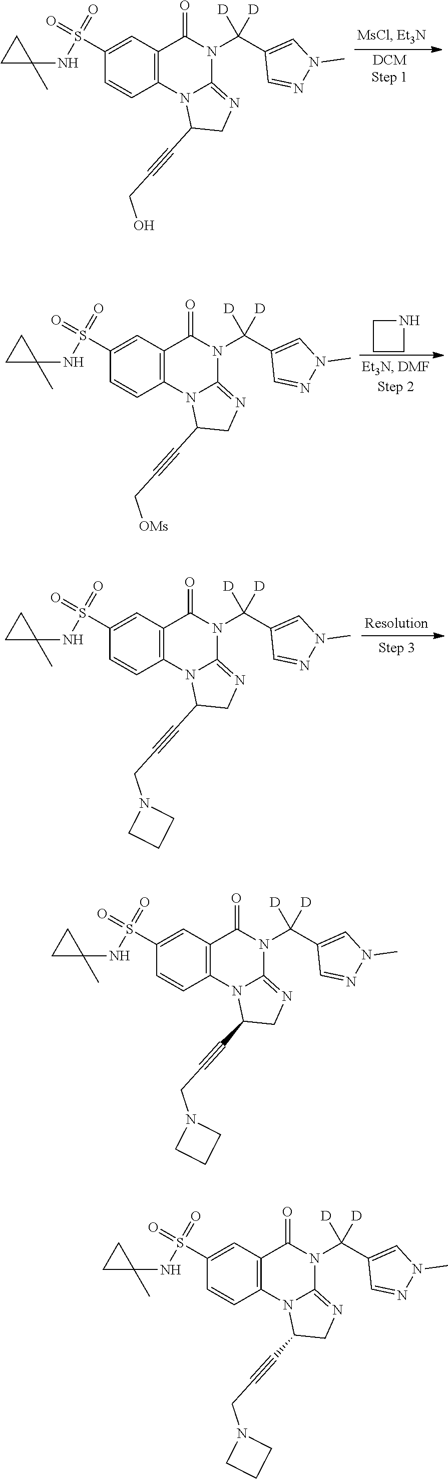

Step 1: To a stirred solution of 1-(3-hydroxyprop-1-yn-1-yl)-N-(1-methylcyclopropyl)-4-[(1-methylpyrazol-4-yl)(2H2)methyl]-5-oxo-1H,2H-imidazo[1,2-a]quinazoline-7-sulfonamide (180 mg, 0.38 mmol) and Et₃N (116.1 mg, 1.14 mmol) in DCM (10 mL) was added MsCl (87.6 mg, 0.76 mmol) at 0° C. The resulting mixture was stirred for 2 h at room temperature. The reaction was quenched by the addition of water (60 mL) at room temperature. The resulting mixture was extracted with CH₂Cl₂ (3×50 mL). The combined organic layers were washed with water (2×50 mL), dried over anhydrous Na₂SO₄. After filtration, the filtrate was concentrated under reduced pressure. The residue was purified by silica gel column chromatography, eluted with CH₂Cl₂/MeOH (0%-10%) to afford 3-{7-[(1-methylcyclopropyl)sulfamoyl]-4-[(1-methylpyrazol-4-yl)(2H2)methyl]-5-oxo-1H,2H-imidazo[1,2-a]quinazolin-1-yl}prop-2-yn-1-yl methanesulfonate (160 mg, 76%) as a yellow solid. LCMS (ESI) m/z: 549 [M+H]⁺.

Step 2: To a stirred mixture of 3-{7-[(1-methylcyclopropyl)sulfamoyl]-4-[(1-methylpyrazol-4-yl)(2H2)methyl]-5-oxo-1H,2H-imidazo[1,2-a]quinazolin-1-yl}prop-2-yn-1-yl methanesulfonate (70 mg, 0.12 mmol) and Et₃N (38.7 mg, 0.38 mmol) in DMF (6 mL) was added azetidine (14.6 mg, 0.25 mmol) at room temperature. The resulting mixture was stirred for 1 h at room temperature. The reaction was quenched by the addition of water (50 mL) at room temperature. The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by silica gel column chromatography, eluted with CH₂Cl₂/MeOH (10:1) to afford 1-[3-(azetidin-1-yl)prop-1-yn-1-yl]-N-(1-methylcyclopropyl)-4-[(1-methylpyrazol-4-yl)(2H2)methyl]-5-oxo-1H,2H-imidazo[1,2-a]quinazoline-7-sulfonamide (60 mg, 92%) as a yellow solid. LCMS (ESI) m/z: 510 [M+H]⁺.

Step 3: The racemate (20 mg) was purified by chiral-Prep-HPLC with the following conditions (Mobile Phase A: Hex (0.1% DEA): EtOH 50:50; Flow rate: 1.67 ml/min mL/min; Gradient: isocratic) to afford (1R)-1-[3-(azetidin-1-yl)prop-1-yn-1-yl]-N-(1-methylcyclopropyl)-4-[(1-methylpyrazol-4-yl)(2H2)methyl]-5-oxo-1H,2H-imidazo[1,2-a]quinazoline-7-sulfonamide (7.2 mg, 36%) and (1 S)-1-[3-(azetidin-1-yl)prop-1-yn-1-yl]-N-(1-methylcyclopropyl)-4-[(1-methylpyrazol-4-yl)(2H2)methyl]-5-oxo-1H,2H-imidazo[1,2-a]quinazoline-7-sulfonamide (6.3 mg, 30%) both as white solids. LCMS (ESI) m/z: 510.25 [M+H]⁺. ¹H NMR (400 MHz, Methanol-d4) δ 8.43 (dd, J=4.0, 2.0 Hz, 1H), 8.07-7.96 (m, 1H), 7.69 (s, 1H), 7.55 (d, J=0.8 Hz, 1H), 7.38-7.30 (m, 1H), 5.42-5.31 (m, 1H), 4.38-4.28 (m, 1H), 4.04 (dd, J=13.2, 4.8 Hz, 1H), 3.82 (d, J=1.2 Hz, 3H), 3.27 (d, J=1.6 Hz, 2H), 3.23 (dd, J=7.6, 6.4 Hz, 4H), 2.04-1.92 (m, 2H), 1.14 (d, J=1.6 Hz, 3H), 0.69 (s, 2H), 0.42 (q, J=2.4, 1.6 Hz, 2H).

Representative Synthesis Route 20: Example 103: (R)-1-(3-aminoprop-1-yn-1-yl)-9-chloro-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

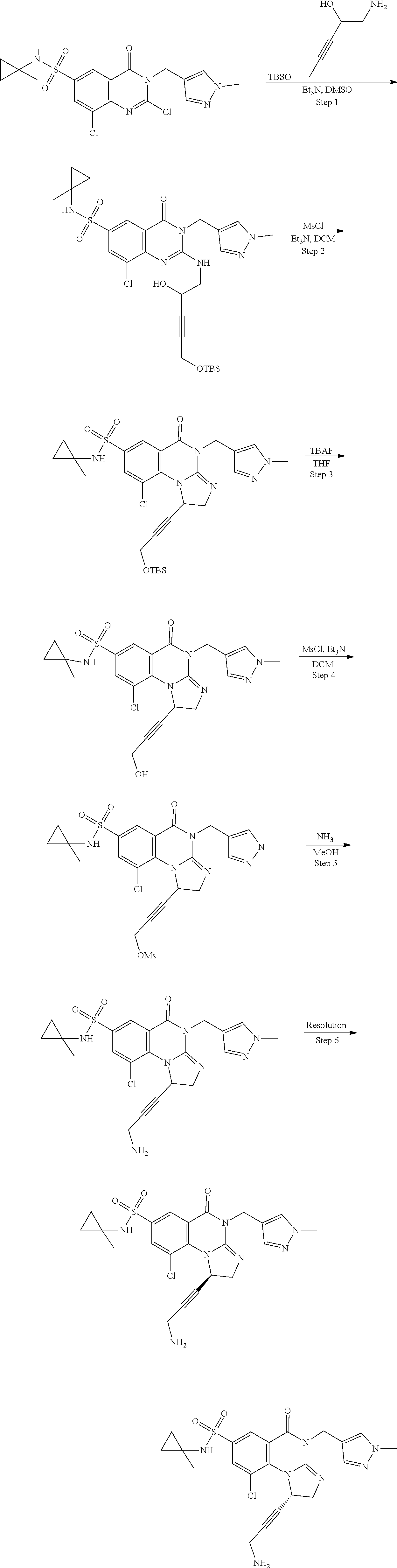

Step 1: A solution of 2,8-dichloro-N-(1-methylcyclopropyl)-3-[(1-methylpyrazol-4-yl)methyl]-4-oxoquinazoline-6-sulfonamide (500 mg, 1.13 mmol) in DMSO (10 mL) was treated with Et₃N (228.8 mg, 2.26 mmol) for 1 min at room temperature under nitrogen atmosphere followed by the addition of 1-amino-5-[(tert-butyldimethylsilyl)oxy]pent-3-yn-2-ol (388.9 mg, 1.69 mmol) in portions at room temperature. The reaction mixture was stirred at room temperature for 2 h. The reaction was quenched by the addition of water (10 mL) at room temperature. The resulting mixture was extracted with EtOAc (20 mL). The combined organic layers were washed with brine (20 mL), dried over anhydrous MgSO₄. After filtration, the filtrate was concentrated and the residue was purified by reverse phase flash chromatography with the following conditions: column, C18 silica gel; mobile phase, MeCN in water (0.1% FA), 10% to 60% gradient in 10 min. This resulted in 2-({5-[(tert-butyldimethylsilyl)oxy]-2-hydroxypent-3-yn-1-yl}amino)-8-chloro-N-(1-methylcyclopropyl)-3-[(1-methylpyrazol-4-yl)methyl]-4-oxoquinazoline-6-sulfonamide (400 mg) as a brown solid.

Step 2: A solution of 2-({5-[(tert-butyldimethylsilyl)oxy]-2-hydroxypent-3-yn-1-yl}amino)-8-chloro-N-(1-methylcyclopropyl)-3-[(1-methylpyrazol-4-yl)methyl]-4-oxoquinazoline-6-sulfonamide (350 mg, 0.55 mmol) in DMSO (10 mL) was treated with Et₃N (111.5 mg, 1.1 mmol) for 1 min at room temperature under nitrogen atmosphere followed by the addition of MsCl (126.2 mg, 1.1 mmol) in portions at room temperature. The reaction mixture was stirred at room temperature for 16 h. The reaction was quenched by the addition of water (10 mL) at room temperature. The resulting mixture was extracted with EtOAc (20 mL). The combined organic layers were washed with brine (20 mL), dried over anhydrous MgSO₄. After filtration, the filtrate was concentrated and the residue was purified by reverse phase flash chromatography with the following conditions: column, C18 silica gel; mobile phase, MeCNin water (0.1% F A), 10% to 50% gradient in 10 min. This resulted in 1-{3-[(tert-butyldimethylsilyl)oxy]prop-1-yn-1-yl}-9-chloro-N-(1-methylcyclopropyl)-4-[(1-methylpyrazol-4-yl)methyl]-5-oxo-1H,2H-imidazo[1,2-a]quinazoline-7-sulfonamide (300 mg) as a brown solid.

Step 3: A solution of 1-{3-[(tert-butyldimethylsilyl)oxy]prop-1-yn-1-yl}-9-chloro-N-(1-methylcyclopropyl)-4-[(1-methylpyrazol-4-yl)methyl]-5-oxo-1H,2H-imidazo[1,2-a]quinazoline-7-sulfonamide (300 mg, 0.49 mmol) and TBAF (254.2 mg, 0.97 mmol) in THF (10 mL) was stirred for 1 h at 0° C. under nitrogen atmosphere. The reaction was quenched by the addition of water (20 mL) at room temperature. The resulting mixture was extracted with EtOAc (50 mL). The combined organic layers were washed with brine (500 mL), dried over anhydrous MgSO₄. After filtration, the filtrate was concentrated and the residue was purified by silica gel column chromatography, eluted with CH₂Cl₂/MeOH (9:1) to afford 9-chloro-1-(3-hydroxyprop-1-yn-1-yl)-N-(1-methylcyclopropyl)-4-[(1-methylpyrazol-4-yl)methyl]-5-oxo-1H,2H-imidazo[1,2-a]quinazoline-7-sulfonamide (200 mg) as a brown solid. LCMS (ESI) m/z: 503 [M+H]⁺.

Step 4: A solution of 9-chloro-1-(3-hydroxyprop-1-yn-1-yl)-N-(1-methylcyclopropyl)-4-[(1-methylpyrazol-4-yl)methyl]-5-oxo-1H,2H-imidazo[1,2-a]quinazoline-7-sulfonamide (200 mg, 0.39 mmol) in DCM (10 mL) was treated with Et₃N (80.5 mg, 0.79 mmol) for 1 min at room temperature under nitrogen atmosphere followed by the addition of MsCl (91 mg, 0.79 mmol) in portions at room temperature. The reaction mixture was stirred at room temperature for 2 h. The reaction was quenched by the addition of water (10 mL) at room temperature. The resulting mixture was extracted with EtOAc (20 mL). The combined organic layers were washed with brine (20 mL), dried over anhydrous MgSO₄. After filtration, the filtrate was concentrated and the residue was purified by silica gel column chromatography, eluted with PE/EA (1:8) to afford 3-{9-chloro-7-[(1-methylcyclopropyl)sulfamoyl]-4-[(1-methylpyrazol-4-yl)methyl]-5-oxo-1H,2H-imidazo[1,2-a]quinazolin-1-yl}prop-2-yn-1-yl methanesulfonate (150 mg) as a brown oil.

Step 5: A solution of 3-{9-chloro-7-[(1-methylcyclopropyl)sulfamoyl]-4-[(1-methylpyrazol-4-yl)methyl]-5-oxo-1H,2H-imidazo[1,2-a]quinazolin-1-yl}prop-2-yn-1-yl methanesulfonate (150 mg, 0.26 mmol) in NH₃ in MeOH (5 mL) was stirred for 1 h at 80° C. under nitrogen atmosphere. After completion of reaction, the reaction mixture was quenched by addition of water (10 mL). The resulting mixture was extracted with EtOAc (50 mL). The combined organic layers were washed with brine (50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% FA), 10% to 50% gradient in 10 min. This resulted in 1-(3-aminoprop-1-yn-1-yl)-9-chloro-N-(1-methylcyclopropyl)-4-[(1-methylpyrazol-4-yl)methyl]-5-oxo-1H,2H-imidazo[1,2-a]quinazoline-7-sulfonamide (80 mg) as a white solid. LCMS (ESI) m/z: 502 [M+H]⁺.

Step 6: The racemate (20 mg) was purified by Prep-CHIRAL-HPLC with the following conditions (Column: CHIRAL ART Cellulose-SZ, 3*25 cm, 5 m; Mobile Phase A: Hex (10 mM NH₃-MeOH), Mobile Phase B: EtOH-HPLC; Flow rate: 40 mL/min; Gradient: isocratic 50; Wave Length: 210/240 nm; Sample Solvent: EtOH: DCM (2:1) to afford (1R)-1-(3-aminoprop-1-yn-1-yl)-9-chloro-N-(1-methylcyclopropyl)-4-[(1-methylpyrazol-4-yl)methyl]-5-oxo-1H,2H-imidazo[1,2-a]quinazoline-7-sulfonamide (3.0 mg) and (1S)-1-(3-aminoprop-1-yn-1-yl)-9-chloro-N-(1-methylcyclopropyl)-4-[(1-methylpyrazol-4-yl)methyl]-5-oxo-1H,2H-imidazo[1,2-a]quinazoline-7-sulfonamide (3.9 mg) both as white solids. LCMS (ESI) m/z: 502.13 [M+H]⁺. ¹H NMR (400 MHz, DMSO-d₆) δ 8.30-8.25 (m, 1H), 8.02-7.94 (m, 1H), 7.70 (s, 1H), 7.42 (s, 1H), 6.20-6.12 (m, 1H), 5.02-4.90 (m, 2H), 4.17-4.07 (m, 1H), 3.93-3.85 (m, 1H), 3.77 (s, 3H), 3.28-3.26 (m, 2H), 1.09 (s, 3H), 0.66-0.57 (m, 2H), 0.46-0.39 (m, 2H).

Representative Synthesis Route 21: Example 107: (S)-4-((1-methyl-1H-pyrazol-4-yl)methyl)-N-(1-methylcyclopropyl)-1-(3-methylisoxazol-5-yl)-5-oxo-1,2,4,5-tetrahydroimidazo[1,2-a]quinazoline-7-sulfonamide

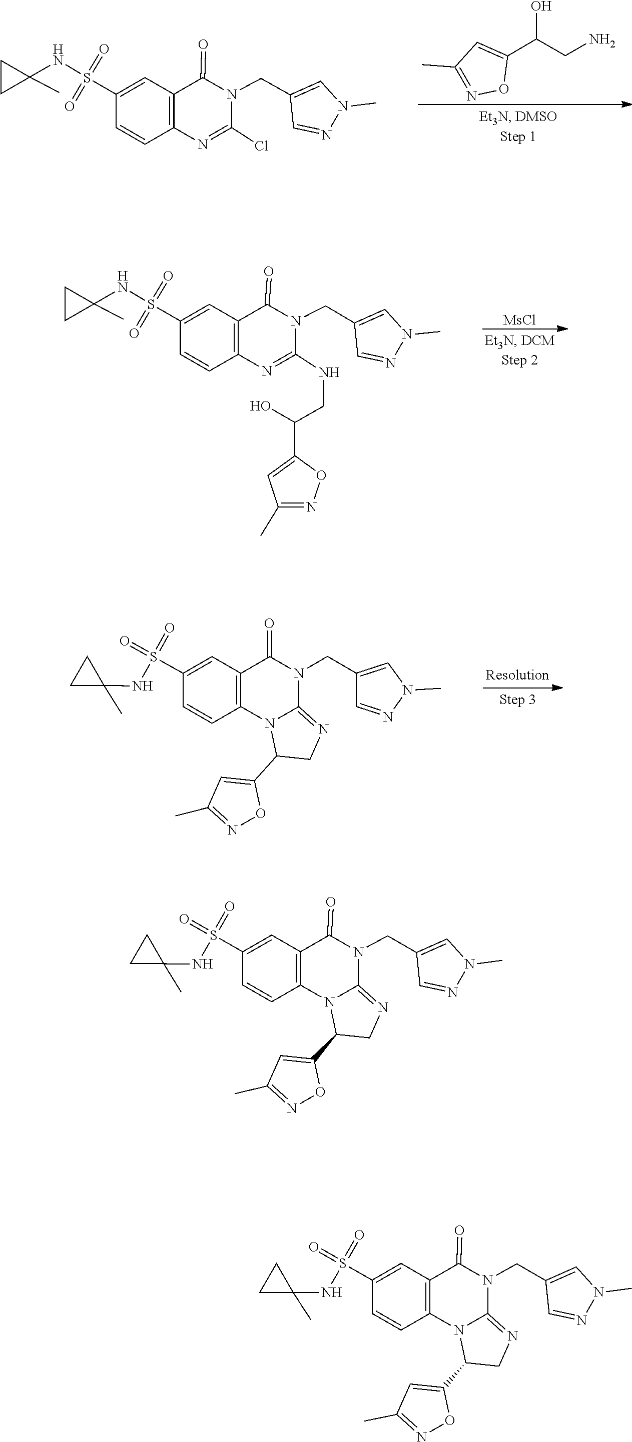

Step 1: To a stirred solution of 2-chloro-N-(1-methylcyclopropyl)-3-[(1-methylpyrazol-4-yl)methyl]-4-oxoquinazoline-6-sulfonamide (200 mg, 0.49 mmol) and 2-amino-1-(3-methyl-1,2-oxazol-5-yl)ethanol (83.6 mg, 0.58 mmol) in DMSO (15 mL) was added Et₃N (148.8 mg, 1.47 mmol) at room temperature. The resulting mixture was stirred for 2 h at room temperature. The reaction was quenched by the addition of water (150 mL) at room temperature. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with CH₂Cl₂/MeOH (0%-10%) to afford (2E)-2-{[2-hydroxy-2-(3-methyl-1,2-oxazol-5-yl)ethyl]imino}-N-(1-methylcyclopropyl)-3-[(1-methylpyrazol-4-yl)methyl]-4-oxo-1H-quinazoline-6-sulfonamide (180 mg, 71%) as a yellow solid. LCMS (ESI) m/z: 514 [M+H]⁺.

Step 2: To a stirred solution of (2E)-2-{[2-hydroxy-2-(3-methyl-1,2-oxazol-5-yl)ethyl]imino}-N-(1-methylcyclopropyl)-3-[(1-methylpyrazol-4-yl)methyl]-4-oxo-1H-quinazoline-6-sulfonamide (180 mg, 0.35 mmol) and MsCl (200.7 mg, 1.75 mmol) in DCM (10 mL) was added Et₃N (354.7 mg, 3.50 mmol) at 0° C. The resulting mixture was stirred for 16 h at room temperature. The reaction was quenched by the addition of water (50 mL) and extracted with CH₂Cl₂ (3×50 mL). The combined organic layers were washed with water (2×50 mL), dried over anhydrous Na₂SO₄. After filtration, the filtrate was concentrated under reduced pressure. The residue was purified by silica gel column chromatography, eluted with CH₂Cl₂/MeOH (5%-15%) to afford 1-(3-methyl-1,2-oxazol-5-yl)-N-(1-methyl(cyclopropyl)-4-[(1-methylpyrazol-4-yl)methyl]-5-oxo-1H,2H-imidazo[1,2-a]quinazoline-7-sulfonamide (100 mg, 57) as a yellow solid. LCMS (ESI) m/z: 496 [M+H].

Step 3: The racemate (100 mg) was purified by Chiral-Prep-HPLC with the following conditions (Column: CHIRALPAK IE, 4.6*50 mm, 3 μm; Mobile Phase A: MTBE (0.1% o DEA): MeOH (70:30); Gradient: isocratic) to afford (1R)-1-(3-methyl-1,2-oxazol-5-yl)-N-(1-methyl cyclopropyl)-4-[(-methylpyrazol-4-yl)methyl]-5-oxo-1H,2H-imidazo[1,2-a]quinazoline-7-sulfonamide (40.3 mg, 400l) and (1r)-1-(3-methyl-1,2-oxazol-5-yl)-N-(1-methylcyclopropyl)-4-[(1-methylpyrazol-4-yl)methyl]-5-oxo-1H,2H-imidazo[1,2-a]quinazoline-7-sulfonamide (41.8 mg, 42) both as white solids. LCMS (ESI) m/z: 496.10 [M+H]⁺. ¹H NMR (400 1 z, Methanol-d₄) δ 8.41 (s, 1H), 7.91-7.83 (i, 1H), 7.70 (s, 1H), 7.57 (s, 1H), 6.97-6.89 (m, 1H), 6.33 (s, 1H), 5.89 (dd, J=10.4, 4.4 Hz, 1H), 5.12-4.98 (m, 2H), 4.44 (dd, J=13.6, 10.4 Hz, 1H), 3.99 (dd, J=13.6, 4.4 Hz, 1H), 3.83 (d, J=1.2 Hz, 3H), 2.25-2.20 (m, 3H), 1.12 (d, J=1.6 Hz, 3H), 0.66 (d, J=5.6 Hz, 2H), 0.44-0.36 (in, 2H).

The following compounds in Table 14 were prepared using procedures similar to those described in Representative Synthesis Route 21 for Example 107 using appropriate starting

TABLE 14

| Example | | |
|---|---|---|
| No. | Name | [M + H]⁺ |
| 115 | (S)-1-(cyclopropoxymethyl)-4-((1-methyl-1H-pyrazol-4- | 485.05 |
| | yl)methyl)-N-(1-methylcyclopropyl)-5-oxo-1,2,4,5- | |
| | tetrahydroimidazo[1,2-a]quinazoline-7-sulfonamide | |
| 118 | (R)-1-cyclobutyl-4-((1-methyl-1H-pyrazol-4-yl)methyl)-N-(1- | 469.05 |
| | methylcyclopropyl)-5-oxo-1,2,4,5-tetrahydroimidazo[1,2- | |
| | a]quinazoline-7-sulfonamide | |
| 119 | (S)-4-((1-methyl-1H-pyrazol-4-yl)methyl)-N-(1- | 496.15 |
| | methylcyclopropyl)-1-(2-methyloxazol-4-yl)-5-oxo-1,2,4,5- | |
| | tetrahydroimidazo[1,2-a]quinazoline-7-sulfonamide | |
| 121 | (S)-4-((1-methyl-1H-pyrazol-4-yl)methyl)-N-(1- | 512.05 |
| | methylcyclopropyl)-1-(2-methylthiazol-5-yl)-5-oxo-1,2,4,5- | |
| | tetrahydroimidazo[1,2-a]quinazoline-7-sulfonamide | |
| 122 | (R)-4-((1-methyl-1H-pyrazol-4-yl)methyl)-N-(1- | 457.15 |
| | methylcyclopropyl)-5-oxo-1-propyl-1,2,4,5- | |
| | tetrahydroimidazo[1,2-a]quinazoline-7-sulfonamide | |
| 124 | (R)-1-((1-aminocyclopropyl)ethynyl)-4-((1-methyl-1H- | 494.20 |
| | pyrazol-4-yl)methyl)-N-(1-methylcyclopropyl)-5-oxo-1,2,4,5- | |
| | tetrahydroimidazo[1,2-a]quinazoline-7-sulfonamide | |
| 130 | (R)-4-methyl-1-((1-methyl-1H-pyrazol-4-yl)ethynyl)-N-(1- | 439.10 |
| | methylcyclopropyl)-5-oxo-1,2,4,5-tetrahydroimidazo[1,2- | |
| | a]quinazoline-7-sulfonamide | |
| 135 | 1-((4-cyanophenoxy)methyl)-4-((1-methyl-1H-pyrazol-4- | 546.00 |
| | yl)methyl)-N-(1-methylcyclopropyl)-5-oxo-1,2,4,5- | |
| | tetrahydroimidazo[1,2-a]quinazoline-7-sulfonamide | |
| 136 | 1-((3-cyanophenoxy)methyl)-4-((1-methyl-1H-pyrazol-4- | 546.00 |
| | yl)methyl)-N-(1-methylcyclopropyl)-5-oxo-1,2,4,5- | |
| | tetrahydroimidazo[1,2-a]quinazoline-7-sulfonamide | |
| 137 | 1-((2-cyanophenoxy)methyl)-4-((1-methyl-1H-pyrazol-4- | 546.00 |
| | yl)methyl)-N-(1-methylcyclopropyl)-5-oxo-1,2,4,5- | |
| | tetrahydroimidazo[1,2-a]quinazoline-7-sulfonamide | |
| 138 | 1-((3-chlorophenoxy)methyl)-4-((1-methyl-1H-pyrazol-4- | 555.00 |
| | yl)methyl)-N-(1-methylcyclopropyl)-5-oxo-1,2,4,5- | |
| | tetrahydroimidazo[1,2-a]quinazoline-7-sulfonamide | |
| 141 | (S)-1-(((1-methyl-1H-pyrazol-3-yl)oxy)methyl)-4-((1-methyl- | 525.15 |
| | 1H-pyrazol-4-yl)methyl)-N-(1-methylcyclopropyl)-5-oxo- | |
| | 1,2,4,5-tetrahydroimidazo[1,2-a]quinazoline-7-sulfonamide | |
| 143 | 1-((4-chlorophenoxy)methyl)-4-((1-methyl-1H-pyrazol-4- | 555.00 |
| | yl)methyl)-N-(1-methylcyclopropyl)-5-oxo-1,2,4,5- | |
| | tetrahydroimidazo[1,2-a]quinazoline-7-sulfonamide | |
| 144 | (R)-4-((1-methyl-1H-pyrazol-4-yl)methyl)-N-(1- | 491.00 |
| | methylcyclopropyl)-5-oxo-1-phenyl-1,2,4,5- | |
| | tetrahydroimidazo[1,2-a]quinazoline-7-sulfonamide | |
| 145 | (7aS,11aR)-6-((1-methyl-1H-pyrazol-4-yl)methyl)-N-(1- | 469.00 |
| | methylcyclopropyl)-5-oxo-5,6,7a,8,9,10,11,11a- | |
| | octahydrobenzo[4,5]imidazo[1,2-a]quinazoline-3-sulfonamide | |
| 150 | 1-((isoxazol-3-yloxy)methyl)-4-((1-methyl-1H-pyrazol-4- | 512.00 |
| | yl)methyl)-N-(1-methylcyclopropyl)-5-oxo-1,2,4,5- | |
| | tetrahydroimidazo[1,2-a]quinazoline-7-sulfonamide | |
| 151 | 1-((2-fluorophenoxy)methyl)-4-((1-methyl-1H-pyrazol-4- | 539.00 |
| | yl)methyl)-N-(1-methylcyclopropyl)-5-oxo-1,2,4,5- | |
| | tetrahydroimidazo[1,2-a]quinazoline-7-sulfonamide | |
| 153 | (R)-1-ethynyl-N-(1-methylcyclopropyl)-4-((2-methyloxazol-5- | 440.20 |
| | yl)methyl)-5-oxo-1,2,4,5-tetrahydroimidazo[1,2- | |
| | a]quinazoline-7-sulfonamide | |
| 154 | (R)-2-((benzyloxy)methyl)-4-((1-methyl-1H-pyrazol-4- | 535.00 |
| | yl)methyl)-N-(1-methylcyclopropyl)-5-oxo-1,2,4,5- | |
| | tetrahydroimidazo[1,2-a]quinazoline-7-sulfonamide | |
| 155 | (S)-4-((1-methyl-1H-pyrazol-4-yl)methyl)-N-(1- | 491.00 |
| | methylcyclopropyl)-5-oxo-2-phenyl-1,2,4,5- | |
| | tetrahydroimidazo[1,2-a]quinazoline-7-sulfonamide | |
| 156 | (S)-2-ethynyl-4-((1-methyl-1H-pyrazol-4-yl)methyl)-N-(1- | 439.00 |
| | methylcyclopropyl)-5-oxo-1,2,4,5-tetrahydroimidazo[1,2- | |
| | a]quinazoline-7-sulfonamide | |
| 157 | (7aS,1laS)-6-((1-methyl-1H-pyrazol-4-yl)methyl)-N-(1- | 471.00 |
| | methylcyclopropyl)-5-oxo-6,7a,8,9,11,11a-hexahydro-5H- | |
| | pyrano[4′,3′:4,5]imidazo[1,2-a]quinazoline-3-sulfonamide | |
| 158 | (7aR,11aR)-6-((1-methyl-1H-pyrazol-4-yl)methyl)-N-(1- | 471.00 |
| | methylcyclopropyl)-5-oxo-5,6,7a,10,11,11a-hexahydro-8H- | |
| | pyrano[3′,4′:4,5]imidazo[1,2-a]quinazoline-3-sulfonamide | |
| 159 | (7aS,11aR)-6-((1-methyl-1H-pyrazol-4-yl)methyl)-N-(1- | 470.00 |
| | methylcyclopropyl)-5-oxo-5,6,7a,8,9,10,11,11a- | |
| | octahydropyrido[3′,4′:4,5]imidazo[1,2-a]quinazoline-3- | |
| | sulfonamide | |
| 160 | (7aS,11aR)-6-((1-methyl-1H-pyrazol-4-yl)methyl)-N-(1- | 470.00 |
| | methylcyclopropyl)-5-oxo-5,6,7a,8,9,10,11,11a- | |
| | octahydropyrido[4′,3′:4,5]imidazo[1,2-a]quinazoline-3- | |
| | sulfonamide | |
| 161 | (7aS,11aR)-N,N-dimethyl-6-((1-methyl-1H-pyrazol-4- | 541.00 |
| | yl)methyl)-3-(N-(1-methylcyclopropyl)sulfamoyl)-5-oxo- | |
| | 6,7a,8,9,11,1la-hexahydropyrido[4′,3′:4,5]imidazo[1,2- | |
| | a]quinazoline-10(5H)-carboxamide | |
| 173 | (1R,2S)-1-ethynyl-2-methyl-4-((1-methyl-1H-pyrazol-4- | 453.00 |
| | yl)methyl)-N-(1-methylcyclopropyl)-5-oxo-1,2,4,5- | |
| | tetrahydroimidazo[1,2-a]quinazoline-7-sulfonamide | |
| 188 | (R)-1-(cyclopropylethynyl)-4-ethyl-N-(1-methylcyclopropyl)- | 413.15 |
| | 5-oxo-1,2,4,5-tetrahydroimidazo[1,2-a]quinazoline-7- | |
| | sulfonamide | |
| 212 | (7aS,10aR)-2-fluoro-6-((1-methyl-1H-pyrazol-4-yl)methyl- | 475.00 |
| | d2)-N-(1-methylcyclopropyl)-5-oxo-6,7a,8,9,10,10a- | |
| | hexahydro-5H-cyclopenta[4,5]imidazo[1,2-a]quinazoline-3- | |
| | sulfonamide | |
| 219 | (R)-1-methyl-5-((1-methyl-1H-pyrazol-4-yl)methyl)-N-(1- | 445.25 |
| | methylcyclopropyl)-6-oxo-1,2,5,6-tetrahydro- | |
| | [1,2,4]oxadiazino[4,3-a]quinazoline-8-sulfonamide | |
| 220 | (S)-1-methyl-5-((1-methyl-1H-pyrazol-4-yl)methyl)-N-(1- | 445.20 |
| | methylcyclopropyl)-6-oxo-1,2,5,6-tetrahydro- | |
| | [1,2,4]oxadiazino[4,3-a]quinazoline-8-sulfonamide | |
| 223 | (R)-4-((2-cyanopyridin-3-yl)methyl)-8-fluoro-1-methyl-N-(1- | 469.00 |
| | methylcyclopropyl)-5-oxo-1,2,4,5-tetrahydroimidazo[1,2- | |
| | a]quinazoline-7-sulfonamide | |
| 229 | (R)-4-methyl-N-(1-methylcyclopropyl)-1-((1-(4- | 497.20 |
| | methylpiperazin-1-yl)cyclopropyl)ethynyl)-5-oxo-1,2,4,5- | |
| | tetrahydroimidazo[1,2-a]quinazoline-7-sulfonamide | |
| 233 | (R)-4-((6-cyanopyridin-3-yl)methyl)-1-methyl-N-(1- | 451.00 |
| | methylcyclopropyl)-5-oxo-1,2,4,5-tetrahydroimidazo[1,2- | |
| | a]quinazoline-7-sulfonamide | |
| 235 | (1S,3R)-1,3-dimethyl-5-((1-methyl-1H-pyrazol-4-yl)methyl)- | 457.15 |
| | N-(1-methylcyclopropyl)-6-oxo-2,3,5,6-tetrahydro-1H- | |
| | pyrimido[1,2-a]quinazoline-8-sulfonamide | |
| 236 | (1R,3R)-1,3-dimethyl-5-((1-methyl-1H-pyrazol-4-yl)methyl)- | 457.20 |
| | N-(1-methylcyclopropyl)-6-oxo-2,3,5,6-tetrahydro-1H- | |
| | pyrimido[1,2-a]quinazoline-8-sulfonamide | |
| 239 | (R)-4-((2-cyanopyridin-4-yl)methyl)-1-methyl-N-(1- | 451.00 |
| | methylcyclopropyl)-5-oxo-1,2,4,5-tetrahydroimidazo[1,2- | |
| | a]quinazoline-7-sulfonamide | |
| 256 | (R)-4-((1-methyl-1H-pyrazol-4-yl)methyl)-N-(1- | 522.25 |
| | methylcyclopropyl)-1-(((R)-1-methylpyrrolidin-2-yl)ethynyl)- | |
| | 5-oxo-1,2,4,5-tetrahydroimidazo[1,2-a]quinazoline-7- | |
| | sulfonamide | |
| 264 | (7aS,10aR)-6-ethyl-2-fluoro-N-(1-methylcyclopropyl)-5-oxo- | 407.00 |
| | 6,7a,8,9,10,10a-hexahydro-5H-cyclopenta[4,5]imidazo[1,2- | |
| | a]quinazoline-3-sulfonamide | |
| 270 | (S)-1-(1-(dimethylamino)cyclopropyl)-4-((1-methyl-1H- | 498.25 |
| | pyrazol-4-yl)methyl)-N-(1-methylcyclopropyl)-5-oxo-1,2,4,5- | |
| | tetrahydroimidazo[1,2-a]quinazoline-7-sulfonamide | |
| 271 | 1-ethynyl-1-methyl-4-((1-methyl-1H-pyrazol-4-yl)methyl)-N- | 453.15 |
| | (1-methylcyclopropyl)-5-oxo-1,2,4,5-tetrahydroimidazo[1,2- | |
| | a]quinazoline-7-sulfonamide | |
| | Absolute stereochemistry not assigned (Isomer A) | |
| 272 | 1-ethynyl-1-methyl-4-((1-methyl-1H-pyrazol-4-yl)methyl)-N- | 453.15 |
| | (1-methylcyclopropyl)-5-oxo-1,2,4,5-tetrahydroimidazo[1,2- | |
| | a]quinazoline-7-sulfonamide | |
| | Absolute stereochemistry not assigned (Isomer B) | |
| 273 | (7aS,10aR)-6-((6-methoxypyridin-3-yl)methyl)-N-(1- | 482.00 |
| | methylcyclopropyl)-5-oxo-6,7a,8,9,10,10a-hexahydro-5H- | |
| | cyclopenta[4,5]imidazo[1,2-a]quinazoline-3-sulfonamide | |
| 276 | (S)-4-((1-methyl-1H-pyrazol-4-yl)methyl)-N-(1- | 498.25 |
| | methylcyclopropyl)-1-((S)-1-methylpyrrolidin-2-yl)-5-oxo- | |
| | 1,2,4,5-tetrahydroimidazo[1,2-a]quinazoline-7-sulfonamide | |
| 283 | (7aR,9R,10aS)-9-amino-6-((1-methyl-1H-pyrazol-4- | 472.25 |
| | yl)methyl-d2)-N-(1-methylcyclopropyl)-5-oxo- | |
| | 6,7a,8,9,10,10a-hexahydro-5H-cyclopenta[4,5]imidazo[1,2- | |
| | a]quinazoline-3-sulfonamide | |

Representative Synthesis Route 22: Example 109: 3-((dimethylamino)methyl)-4′-((1-methyl-1H-pyrazol-4-yl)methyl-d2)-N-(1-methylcyclopropyl)-5′-oxo-4′,5′-dihydro-2′H-spiro[cyclobutane-1,1′-imidazo[1,2-a]quinazoline]-7′-sulfonamide

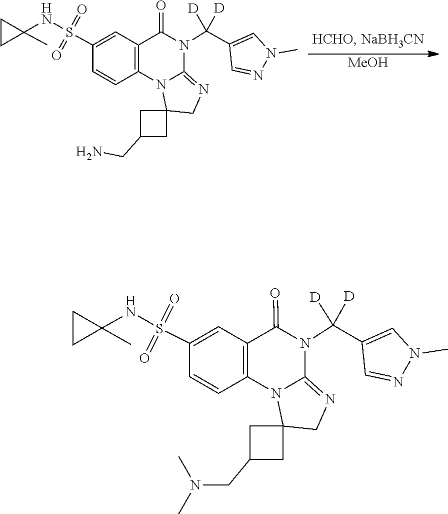 [SMILES: "MeOH" ; [2H]C([2H])(c1cnn(C)c1)N1C(=O)c2cc(S(=O)(=O)NC3(C)CC3)ccc2N2C1=NCC21CC(CN)C1 ; [2H]C([2H])(c1cnn(C)c1)N1C(=O)c2cc(S(=O)(=O)NC3(C)CC3)ccc2N2C1=NCC21CC(CN(C)C)C1]

A solution of 3-(aminomethyl)-N-(1-methylcyclopropyl)-4′-[(1-methylpyrazol-4-yl)(2H2)methyl]-5′-oxo-2′H-spiro[cyclobutane-1,1′-imidazo[1,2-a]quinazoline]-7′-sulfonamide (100 mg, 0.21 mmol) and HCHO (30.9 mg, 1.03 mmol) in MeOH (5 mL) was stirred for 30 min at room temperature. To the above mixture was added NaBH₃CN (38.8 mg, 0.62 mmol) in portions over 2 min at room temperature. The resulting mixture was stirred at room temperature overnight. The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by reversed -phase flash chromatography with the following conditions: column, C18 silica gel; mobile phase, MeOH in water (0.1% NH₃·H₂O+10 mmol/L NH₄HCO₃), 10% to 50% gradient in 10 min; detector, UV 254 nm to obtain 3-[(dimethylamino)methyl]-N-(1-methylcyclopropyl)-4′-[(1-methylpyrazol-4-yl)(2H2)methyl]-5′-oxo-2′H-spiro[cyclobutane-1,1′-imidazo[1,2-a]quinazoline]-7′-sulfonamide) as a white solid (23.7 mg, 22%). LCMS (ESI) m/z: 514.25 [M+H]⁺. ¹H NMR (400 MHz, DMSO-d₆) δ 8.47-8.26 (m, 2H), 8.04 (dd, J=8.8, 2.4 Hz, 1H), 7.69 (s, 1H), 7.58 (d, J=8.9 Hz, 1H), 7.47 (s, 1H), 4.16 (s, 2H), 3.77 (s, 3H), 3.51-3.26 (m, 2H), 3.15 (d, J=7.6 Hz, 2H), 3.02 (s, 1H), 2.69 (s, 6H), 2.20-1.99 (m, 2H), 1.11 (s, 3H), 0.63 (d, J=5.3 Hz, 2H), 0.44 (q, J=5.2, 4.8 Hz, 2H).

Representative Synthesis Route 23: Example 110: (S)-4-((1-methyl-1H-pyrazol-4-yl)methyl-d2)-N-(1-methylcyclopropyl)-5-oxo-1-((pyridin-2-yloxy)methyl)-1,2,4,5-tetrahydroimidazo[1,2-a]quinazoline-7-sulfonamide

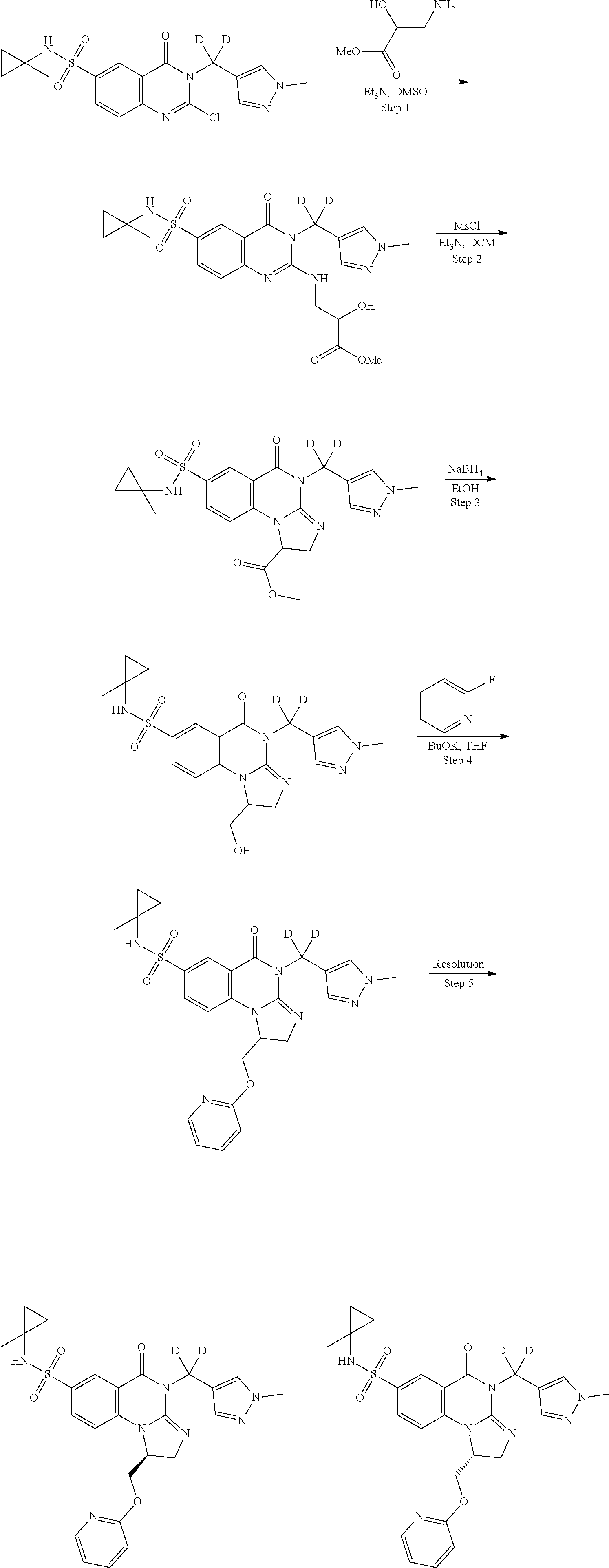

Step 1: To a stirred solution of 2-chloro-N-(1-methylcyclopropyl)-3-[(1-methylpyrazol-4-yl)(2H2)methyl]-4-oxoquinazoline-6-sulfonamide (400 mg, 0.97 mmol) and methyl 3-amino-2-hydroxypropanoate (139 mg, 1.17 mmol) in DMSO (10 mL) was added Et₃N (296 mg, 2.92 mmol) at room temperature. The resulting mixture was stirred for 2 h at room temperature. The reaction was quenched by the addition of water (200 mL) at room temperature. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with MeOH/CH₂Cl₂ (0%-10%) to afford methyl 2-hydroxy-3-{[(2E)-6-[(1-methylcyclopropyl)sulfamoyl]-3-[(1-methylpyrazol-4-yl)(2H2)methyl]-4-oxo-1H-quinazolin-2-ylidene]amino}propanoate (450 mg, 94%) as a yellow solid. LCMS (ESI) m/z: 493 [M+H]⁺.

Step 2: To a stirred solution of methyl 2-hydroxy-3-{[(2E)-6-[(1-methylcyclopropyl)sulfamoyl]-3-[(1-methylpyrazol-4-yl)(2H2)methyl]-4-oxo-1H-quinazolin-2-ylidene]amino}propanoate (450 mg, 0.91 mmol) and Et₃N (1.38 g, 13.7 mmol) in DCM (10 mL) was added MsCl (1.04 g, 9.14 mmol) at 0° C. The resulting mixture was stirred for 2 h at room temperature. The reaction was quenched by the addition of water (200 mL) at room temperature. The resulting mixture was extracted with CH₂Cl₂ (3×100 mL). The combined organic layers were washed with water (2×100 mL), dried over anhydrous Na₂SO₄. After filtration, the filtrate was concentrated under reduced pressure. The residue was purified by silica gel column chromatography, eluted with MeOH/CH₂Cl₂ (5%-15%) to afford methyl 7-[(1-methylcyclopropyl)sulfamoyl]-4-[(1-methylpyrazol-4-yl)(2H2)methyl]-5-oxo-1H,2H-imidazo[1,2-a]quinazoline-1-carboxylate (340 mg, 78%) as a yellow solid. LCMS (ESI) m/z: 475 [M+H]⁺.

Step 3: A solution of methyl 7-[(1-methylcyclopropyl)sulfamoyl]-4-[(1-methylpyrazol-4-yl)(2H2)methyl]-5-oxo-1H,2H-imidazo[1,2-a]quinazoline-1-carboxylate (330 mg, 0.69 mmol) and NaBH₄ (52 mg, 1.39 mmol) in EtOH (10 mL) was stirred for 2 h at room temperature. The reaction was quenched by the addition of water (100 mL) at 0° C. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with CH₂Cl₂/MeOH (5%-15%) to afford 1-(hydroxymethyl)-N-(1-methylcyclopropyl)-4-[(1-methylpyrazol-4-yl)(2H2)methyl]-5-oxo-1H,2H-imidazo[1,2-a]quinazoline-7-sulfonamide (270 mg, 87%) as a yellow solid. LCMS (ESI) m/z: 447 [M+H]⁺.

Step 4: To a stirred solution of 1-(hydroxymethyl)-N-(1-methylcyclopropyl)-4-[(1-methylpyrazol-4-yl)(2H2)methyl]-5-oxo-1H,2H-imidazo[1,2-a]quinazoline-7-sulfonamide (90 mg, 0.20 mmol) and 2-fluoropyridine (58 mg, 0.60 mmol) in THF (5 mL) was added t-BuOK (45 mg, 0.40 mmol) at room temperature. The resulting mixture was stirred for 3 h at 65° C. The reaction was quenched by the addition of water (30 mL) at room temperature. The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by silica gel column chromatography, eluted with MeOH/CH₂Cl₂ (5%-15%) to afford N-(1-methylcyclopropyl)-4-[(1-methylpyrazol-4-yl)(2H2)methyl]-5-oxo-1-[(pyridin-2-yloxy)methyl]-1H,2H-imidazo[1,2-a]quinazoline-7-sulfonamide (80 mg, 76%) as a yellow solid. LCMS (ESI) m/z: 524 [M+H]⁺.

Step 5: The racemate (60 mg) was purified by Chiral-Prep-HPLC with the following conditions (Mobile Phase A: MTBE (0.1% DEA): MeOH (90:10); Flow rate: 1.67 ml/min mL/min; Gradient: isocratic) to afford (1R)-N-(1-methylcyclopropyl)-4-[(1-methylpyrazol-4-yl)(2H2)methyl]-5-oxo-1-[(pyridin-2-yloxy)methyl]-1H,2H-imidazo[1,2-a]quinazoline-7-sulfonamide (25.2 mg, 41%) and (1S)-N-(1-methylcyclopropyl)-4-[(1-methylpyrazol-4-yl)(2H2)methyl]-5-oxo-1-[(pyridin-2-yloxy)methyl]-1H,2H-imidazo[1,2-a]quinazoline-7-sulfonamide (24.6 mg, 41%) both as white solids. LCMS (ESI) m/z: 524.15 [M+H]⁺. ¹H NMR (400 MHz, Methanol-d₄) δ 8.35 (s, 1H), 8.03-7.94 (m, 1H), 7.84-7.78 (m, 1H), 7.64 (d, J=1.6 Hz, 1H), 7.56-7.46 (m, 2H), 7.40 (s, 1H), 6.84-6.76 (m, 1H), 6.55-6.48 (m, 1H), 5.04 (s, 1H), 4.81-4.76 (m, 1H), 4.51-4.43 (m, 1H), 4.17 (dd, J=13.6, 9.6 Hz, 1H), 4.06-3.98 (m, 1H), 3.84 (d, J=1.6 Hz, 3H), 1.14 (d, J=2.0 Hz, 3H), 0.68-0.69 (m, 2H), 0.41 (q, J=2.4 Hz, 2H).

Representative Synthesis Route 24: Example 111: (R)-9-chloro-1-ethynyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide

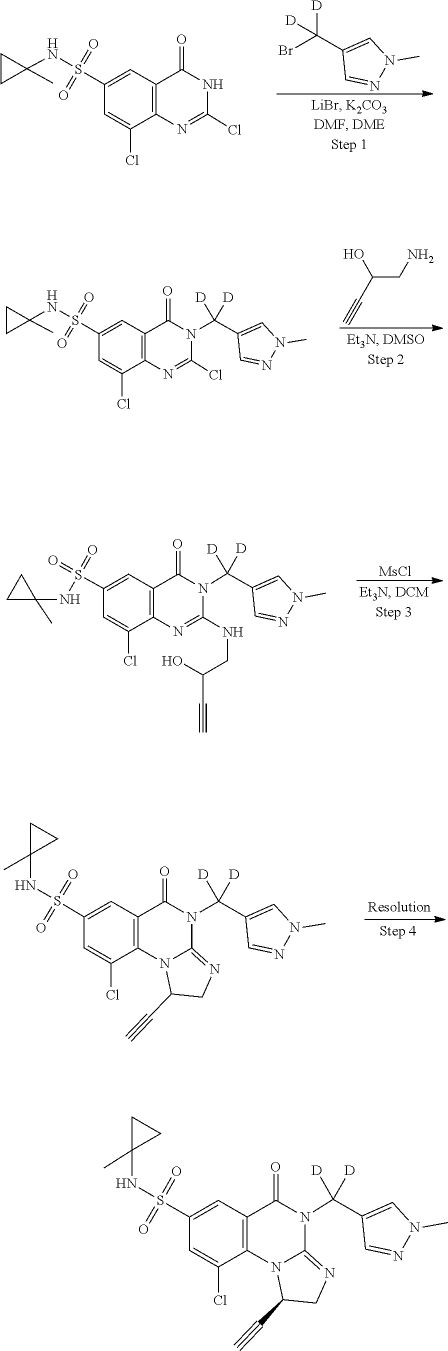

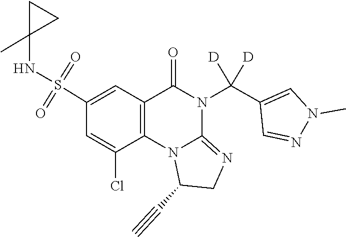

Step 1: To a stirred mixture of 2,8-dichloro-N-(1-methylcyclopropyl)-4-oxo-3H-quinazoline-6-sulfonamide (320 mg, 0.92 mmol) and 4-[bromo(2H2)methyl]-1-methylpyrazole (244 mg, 1.38 mmol) in DME (36 mL) and DMF (9 mL) were added LiBr (159.6 mg, 1.84 mmol) and K₂CO₃ (508 mg, 3.67 mmol) in portions at room temperature. The resulting mixture was stirred for 16 h at room temperature. The reaction was quenched by the addition of water (100 mL) at room temperature. The resulting mixture was extracted with EtOAc (2×100 mL). The combined organic layers were washed with brine (100 mL), dried over anhydrous Na₂SO₄. After filtration, the filtrate was concentrated under reduced pressure. The residue was purified by silica gel column chromatography, eluted with EA/PE (10-40%) to afford 2,8-dichloro-N-(1-methylcyclopropyl)-3-[(1-methylpyrazol-4-yl)(2H2)methyl]-4-oxoquinazoline-6-sulfonamide (200 mg, 49%) as a yellow oil.

Step 2: To a stirred mixture of 2,8-dichloro-N-(1-methylcyclopropyl)-3-[(1-methylpyrazol-4-yl)(2H2)methyl]-4-oxoquinazoline-6-sulfonamide (200 mg, 0.45 mmol) and 1-aminobut-3-yn-2-ol (46 mg, 0.54 mmol) in DMSO (15 mL) was added Et₃N (136.6 mg, 1.35 mmol) dropwise at room temperature. The resulting mixture was stirred at room temperature for 2 h. The reaction was quenched by the addition of water (50 mL) at room temperature. The resulting mixture was extracted with EtOAc (2×50 mL). The combined organic layers were washed with brine (50 mL), dried over anhydrous Na₂SO₄. After filtration, the filtrate was concentrated under reduced pressure. The residue was purified by silica gel column chromatography, eluted with EA/PE (10-30%) to afford 8-chloro-2-((2-hydroxybut-3-yn-1-yl)amino)-3-((1-methyl-1H-pyrazol-4-yl)methyl-d2)-N-(1-methylcyclopropyl)-4-oxo-3,4-dihydroquinazoline-6-sulfonamide (150 mg, 68%) as a yellow oil. LCMS (ESI) m/z: 493 [M+H]⁺.

Step 3: To a stirred mixture of 8-chloro-2-((2-hydroxybut-3-yn-1-yl)amino)-3-((1-methyl-1H-pyrazol-4-yl)methyl-d2)-N-(1-methylcyclopropyl)-4-oxo-3,4-dihydroquinazoline-6-sulfonamide (100 mg, 0.20 mmol) and MsCl (232.3 mg, 2.03 mmol) in DCM (10 mL) was added Et₃N (205.3 mg, 2.03 mmol) dropwise at room temperature. The resulting mixture was stirred at room temperature for 16 h. The resulting mixture was concentrated under reduced pressure. The residue was purified by silica gel column chromatography, eluted with EA/PE (10-30%) to afford 9-chloro-1-ethynyl-N-(1-methylcyclopropyl)-4-[(1-methylpyrazol-4-yl)(2H2)methyl]-5-oxo-1H,2H-imidazo[1,2-a]quinazoline-7-sulfonamide (70 mg, 74%) as a yellow oil. LCMS (ESI) m/z: 475 [M+H]⁺.

Step 4: The racemate (150 mg) was purified by Prep-Chiral-HPLC with the following conditions (Column: CHIRALPAK-IK, 3*25 mm, 5 m; Mobile Phase A: Hex (10 mM NH₃-MeOH), Mobile Phase B: EtOH; Flow rate: 40 mL/min; Gradient: isocratic 40; Wave Length: 202/220 nm; RT1 (min): 23.6; RT2 (min): 31.2; Sample Solvent: EtOH: DCM=1:1) to afford (R)-9-chloro-1-ethynyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide (43.2 mg, 29%) and (S)-9-chloro-1-ethynyl-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide (39.8 mg, 26%) both as white solids. LCMS (ESI) m/z: 475.00 [M+H]⁺. ¹H NMR (400 MHz, DMSO-d₆) δ 8.29-8.23 (m, 2H), 8.00 (d, J=2.2 Hz, 1H), 7.70 (d, J=0.8 Hz, 1H), 7.42 (d, J=0.8 Hz, 1H), 6.17-6.15 (m, 1H), 4.11 (dd, J=13.8, 9.4 Hz, 1H), 3.92 (dd, J=13.6, 2.2 Hz, 1H), 3.77 (s, 3H), 3.60 (dd, J=2.2, 0.8 Hz, 1H), 1.09 (s, 3H), 0.61 (q, J=4.4, 4.0 Hz, 2H), 0.47-0.38 (m, 2H).

The following compounds in Table 15 were prepared using procedures similar to those described in Representative Synthesis Route 24 for Example 111 using appropriate starting materials.

TABLE 15

| Example | | |
|---|---|---|
| No. | Name | [M + H]⁺ |
| 98 | (R)-9-bromo-1-ethynyl-4-((1-methyl-1H-pyrazol-4-yl)methyl- | 519.07, |
| | d2)-N-(1-methylcyclopropyl)-5-oxo-1,2,4,5- | 521.07 |
| | tetrahydroimidazo[1,2-a]quinazoline-7-sulfonamide | |

Representative Synthesis Route 25: Example 112: (R)-1-((1-(dimethylamino)cyclopropyl)ethynyl)-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide

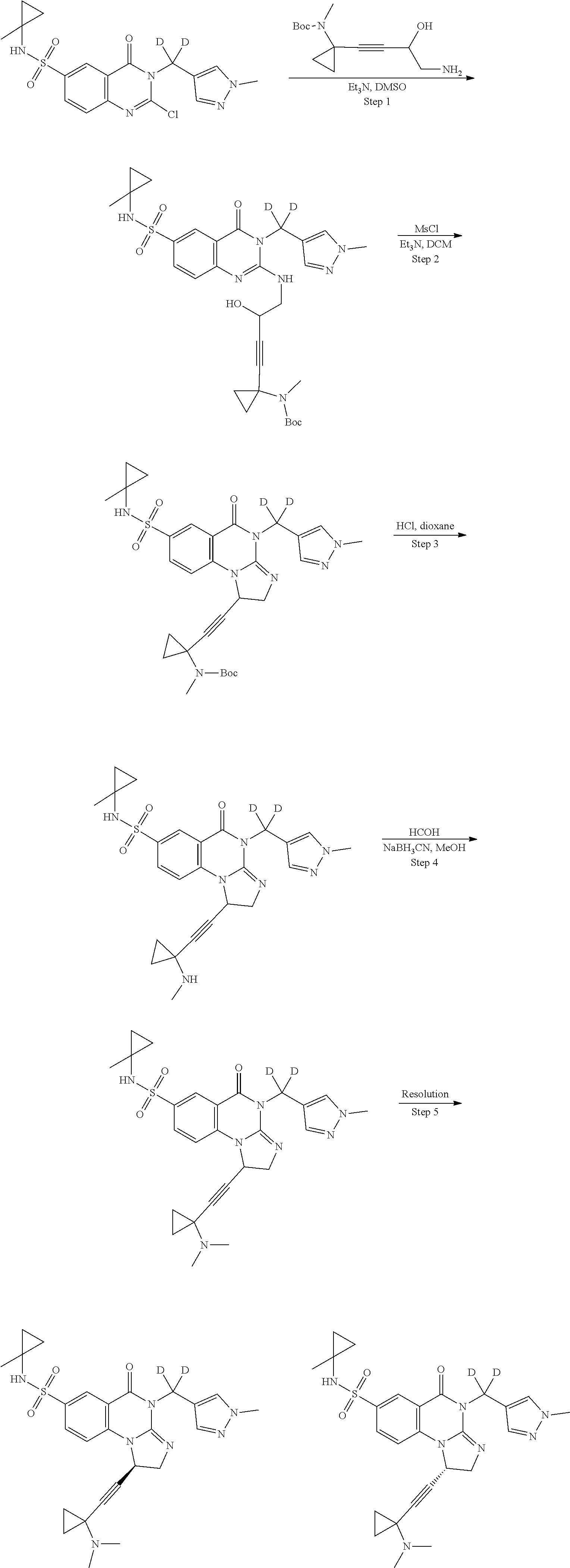

Step 1: A solution of 2-chloro-N-(1-methylcyclopropyl)-3-[(1-methylpyrazol-4-yl)(2H2)methyl]-4-oxoquinazoline-6-sulfonamide (500 mg, 1.22 mmol) in DMSO (15 mL) was treated with Et₃N (246.8 mg, 2.44 mmol) for 1 min at room temperature under nitrogen atmosphere followed by the addition of tert-butyl N-[1-(4-amino-3-hydroxybut-1-yn-1-yl)cyclopropyl]-N-methylcarbamate (372.3 mg, 1.46 mmol) in portions at room temperature. The reaction mixture was stirred at room temperature for 2 h. The reaction was quenched by the addition of water (20 mL) at room temperature. The resulting mixture was extracted with EtOAc (100 mL). The combined organic layers were washed with brine (200 mL), dried over anhydrous MgSO₄. After filtration, the filtrate was concentrated under reduced pressure. The residue was purified by silica gel column chromatography, eluted with CH₂Cl₂/MeOH (9:1) to obtain tert-butyl (1-(3-hydroxy-4-((3-((1-methyl-1H-pyrazol-4-yl)methyl-d2)-6-(N-(1-methylcyclopropyl)sulfamoyl)-4-oxo-3,4-dihydroquinazolin-2-yl)amino)but-1-yn-1-yl)cyclopropyl)(methyl)carbamate (400 mg) as a brown solid. LCMS (ESI) m/z: 628 [M+H]⁺.

Step 2: A solution tert-butyl (1-(3-hydroxy-4-((3-((1-methyl-1H-pyrazol-4-yl)methyl-d2)-6-(N-(1-methylcyclopropyl)sulfamoyl)-4-oxo-3,4-dihydroquinazolin-2-yl)amino)but-1-yn-1-yl)cy clopropyl)(methyl)carbamate (400 mg, 0.64 mmol) in DCM (20 mL) was treated with Et₃N (128.9 mg, 1.27 mmol) for 1 min at room temperature under nitrogen atmosphere followed by the addition of MsCl (145.9 mg, 1.27 mmol) in portions at room temperature. The reaction mixture was stirred at room temperature for 2 h. The reaction was quenched by the addition of water (30 mL) at room temperature. The resulting mixture was extracted with EtOAc (100 mL). The combined organic layers were washed with brine (300 mL), dried over anhydrous MgSO₄. After filtration, the filtrate was concentrated under reduced pressure. The residue was purified by silica gel column chromatography, eluted with CH₂Cl₂/MeOH (9:1) to tert-butyl methyl(1-((4-((1-methyl-1H-pyrazol-4-yl)methyl-d2)-7-(N-(1-methylcyclopropyl)sulfamoyl)-5-oxo-1,2,4,5-tetrahydroimidazo[1,2-a]quinazolin-1-yl)ethynyl)cyclopropyl)carbamate (300 mg) as a brown solid. LCMS (ESI) m/z: 610 [M+H]⁺.

Step 3: A solution of tert-butyl methyl(1-((4-((1-methyl-1H-pyrazol-4-yl)methyl-d2)-7-(N-(1-methylcyclopropyl)sulfamoyl)-5-oxo-1,2,4,5-tetrahydroimidazo[1,2-a]quinazolin-1-yl)ethynyl)cyclopropyl)carbamate (300 mg, 0.48 mmol), HCl in 1,4-dioxane (15 mL)) was stirred for 1 h at room temperature. The reaction was quenched by the addition of water (30 mL) at room temperature. The resulting mixture was extracted with EtOAc (100 mL). The combined organic layers were washed with brine (300 mL), dried over anhydrous MgSO₄. After filtration, the filtrate was concentrated under reduced pressure. The residue was purified by reversed -phase flash chromatography with the following conditions: column, C18 silica gel; mobile phase, MeCN in water (0.1% FA), 10% to 30% gradient in 40 min to obtain 4-((1-methyl-1H-pyrazol-4-yl)methyl-d2)-1-((1-(methylamino)cyclopropyl)ethynyl)-N-(1-methylcyclopropyl)-5-oxo-1,2,4,5-tetrahydroimidazo[1,2-a]quinazoline-7-sulfonamide (180 mg) as a brown oil. LCMS (ESI) m/z: 510 [M+H]⁺.

Step 4: A solution of 4-((1-methyl-1H-pyrazol-4-yl)methyl-d2)-1-((1-(methylamino)cyclopropyl)ethynyl)-N-(1-methylcyclopropyl)-5-oxo-1,2,4,5-tetrahydroimidazo[1,2-a]quinazoline-7-sulfonamide (180 mg, 0.35 mmol) in MeOH (20 mL) was treated with formaldehyde (12.7 mg, 0.42 mmol) for 30 min at room temperature under nitrogen atmosphere followed by the addition of NaBH₃CN (44.4 mg, 0.71 mmol) in portions at 0° C. The resulting mixture was stirred for 1 h at 0° C. under nitrogen atmosphere. The reaction was quenched by the addition of water (30 mL) at room temperature. The resulting mixture was extracted with CH₂Cl₂ (100 mL). The combined organic layers were washed with brine (300 mL), dried over anhydrous MgSO₄. After filtration, the filtrate was concentrated under reduced pressure. The residue was purified by silica gel column chromatography, eluted with CH₂Cl₂/MeOH (9:1) to afford 1-((1-(dimethylamino)cyclopropyl)ethynyl)-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide (60 mg) as a brown solid. LCMS (ESI) m/z: 524 [M+H]⁺.

Step 5: The racemate (60 mg) was purified by Prep-CHIRAL-HPLC with the following conditions (Mobile Phase A: Hex (0.1% DEA): EtOH (50:50); Flow rate: 1.67 ml/min mL/min; Gradient: isocratic) to afford (R)-1-((1-(dimethylamino)cyclopropyl)ethynyl)-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide (27.4 mg, 48%) and (S)-1-((1-(dimethylamino)cyclopropyl)ethynyl)-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide (24.9 mg, 430) both as white solids. LCMS (ESI) m/z: 524.20 [M+H]⁺. ¹H NMR (400 H+z, Methanol-d₄) δ 8.45-8.41 (m, 1H), 8.05-8.00 (m, 1H), 7.68 (s, 1H), 7.55 (s, 1H), 7.36-7.30 (m, 1H), 5.41-5.32 (m, 1H), 4.36-4.26 (m, 1H), 4.03-3.95 (m, 1H), 3.82 (s, 3H), 2.20 (s, 6H), 1.13 (s, 3H), 0.90-0.79 (min, 4H), 0.72-0.65 (m, 2H), 0.45-0.36 (i, 2H).

The following compounds in Table 16 were prepared using procedures similar to those described in Representative Synthesis Route 25 for Example 112 using appropriate starting materials.

TABLE 16

| Example | | |
|---|---|---|
| No. | Name | [M + H]⁺ |
| 127 | (R)-4-((1-methyl-1H-pyrazol-4-yl)methyl)-1-((1- | 508.05 |
| | (methylamino)cyclopropyl)ethynyl)-N-(1-methylcyclopropyl)- | |
| | 5-oxo-1,2,4,5-tetrahydroimidazo[1,2-a]quinazoline-7- | |
| | sulfonamide | |
| 129 | (R)-1-((1-(dimethylamino)cyclopropyl)ethynyl)-4-methyl-N- | 442.10 |
| | (1-methylcyclopropyl)-5-oxo-1,2,4,5-tetrahydroimidazo[1,2- | |
| | a]quinazoline-7-sulfonamide | |
| 186 | (R)-1-((1-(dimethylamino)cyclopropyl)ethynyl)-4-ethyl-N-(1- | 456.10 |
| | methylcyclopropyl)-5-oxo-1,2,4,5-tetrahydroimidazo[1,2- | |
| | a]quinazoline-7-sulfonamide | |
| 241 | (R)-1-((1-(diethylamino)cyclopropyl)ethynyl)-4-((1-methyl- | 552.30 |
| | 1H-pyrazol-4-yl)methyl-d2)-N-(1-methylcyclopropyl)-5-oxo- | |
| | 1,2,4,5-tetrahydroimidazo[1,2-a]quinazoline-7-sulfonamide | |

Representative Synthesis Route 26: Example 114: (S)-1-((2-(dimethylamino)ethoxy)methyl) -4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide

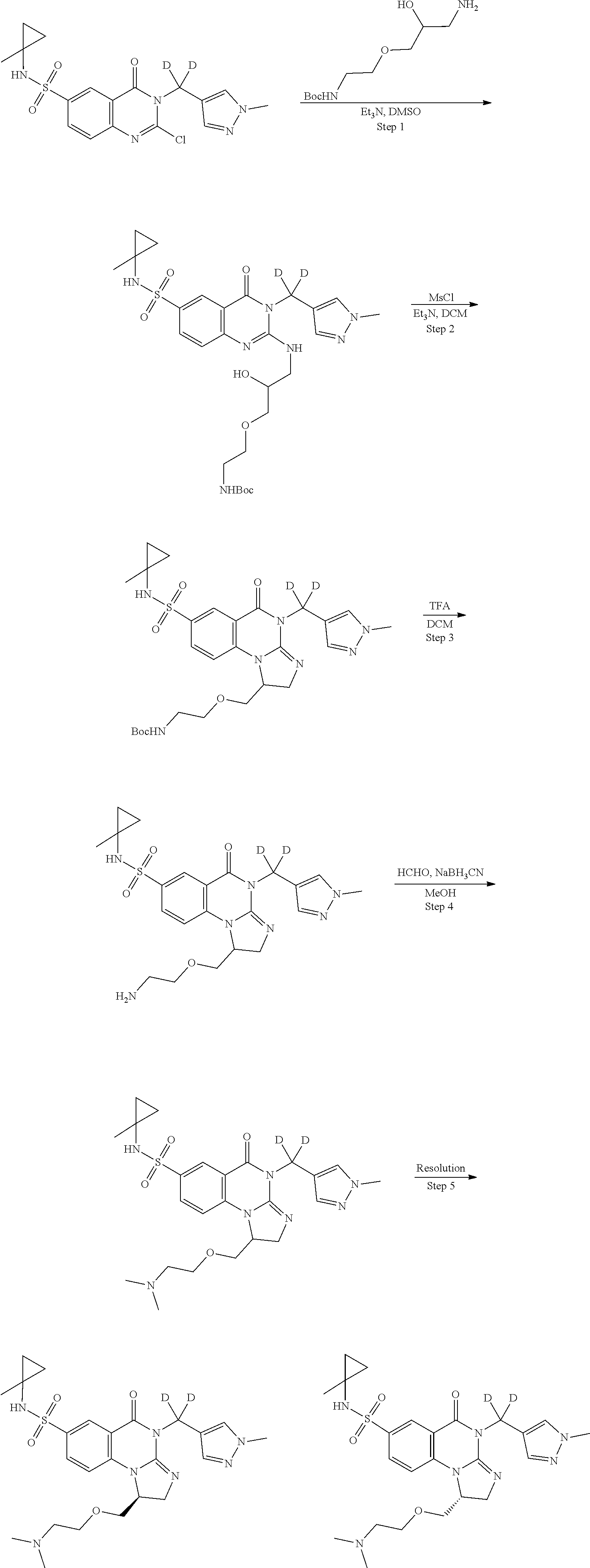

Step 1: A solution of 2-chloro-N-(1-methylcyclopropyl)-3-[(1-methylpyrazol-4-yl)(2H2)methyl]-4-oxoquinazoline-6-sulfonamide (200 mg, 0.49 mmol) in DMSO (10 mL) was treated with Et₃N (98.7 mg, 0.98 mmol) for 1 min at room temperature under nitrogen atmosphere followed by the addition of tert-butyl N-[2-(3-amino-2-hydroxypropoxy)ethyl]carbamate (114.3 mg, 0.49 mmol) in portions at room temperature. The reaction mixture was stirred at room temperature for 2 h. The reaction was quenched by the addition of water (20 mL) at room temperature. The resulting mixture was extracted with EtOAc (100 mL). The combined organic layers were washed with brine (200 mL), dried over anhydrous MgSO₄. After filtration, the filtrate was concentrated under reduced pressure.

The residue was purified by silica gel column chromatography, eluted with CH₂Cl₂/MeOH (9:1) to afford tert-butylN-[2-(2-hydroxy-3-{[(2E)-6-[(1-methylcyclopropyl)sulfamoyl]-3-[(1-methylpyrazol-4-yl)(2H2)methyl]-4-oxo-1H-quinazolin-2-ylidene]amino}propoxy)ethyl]carbamate (180 mg) as a brown solid. LCMS (ESI) m/z: 608 [M+H]⁺.

Step 2: A solution of tert-butyl N-[2-(2-hydroxy-3-{[(2E)-6-[(1-methylcyclopropyl)sulfamoyl]-3-[(1-methylpyrazol-4-yl)(2H2)methyl]-4-oxo-1H-quinazolin-2-ylidene]amino}propoxy)ethyl]carbamate (180 mg, 0.29 mmol) in DCM (10 mL) was treated with Et₃N (59.9 mg, 0.59 mmol) for 1 min at room temperature under nitrogen atmosphere followed by the addition of methanesulfonyl chloride (67.8 mg, 0.59 mmol) at room temperature. The reaction mixture was stirred at room temperature for 2 h. The reaction was quenched by the addition of water (20 mL) at room temperature. The resulting mixture was extracted with EtOAc (100 mL). The combined organic layers were washed with brine (200 mL), dried over anhydrous MgSO₄. After filtration, the filtrate was concentrated under reduced pressure. The residue was purified by silica gel column chromatography, eluted with CH₂Cl₂/MeOH (9:1) to afford tert-butyl N-[2-({7-[(1-methylcyclopropyl)sulfamoyl]-4-[(1-methylpyrazol-4-yl)(2H2)methyl]-5-oxo-1H,2H-imidazo[1,2-a]quinazolin-1-yl}methoxy)ethyl]carbamate (120 mg) as a brown oil. LCMS (ESI) m/z: 590 [M+H]⁺.

Step 3: A solution of tert-butyl N-[2-({7-[(1-methylcyclopropyl)sulfamoyl]-4-[(1-methylpyrazol-4-yl)(2H2)methyl]-5-oxo-1H,2H-imidazo[1,2-a]quinazolin-1-yl}methoxy)ethyl]carbamate (120 mg, 0.20 mmol) and TFA (2 mL) in DCM (10 mL) was stirred at room temperature for 1 h under nitrogen atmosphere. The reaction was quenched by the addition of water (20 mL) at room temperature. The resulting mixture was extracted with EtOAc (100 mL). The combined organic layers were washed with brine (200 mL), dried over anhydrous MgSO₄. After filtration, the filtrate was concentrated under reduced pressure. The residue was purified by reversed-phase flash chromatography with the following conditions: column, C18 silica gel; mobile phase, MeCN in water (0.1% FA), 5% to 50% gradient in 30 min. After filtration, the filtrate was concentrated under reduced pressure. This resulted in 1-[(2-aminoethoxy)methyl]-N-(1-methylcyclopropyl)-4-[(1-methylpyrazol-4-yl)(2H2)methyl]-5-oxo-1H,2H-imidazo[1,2-a]quinazoline-7-sulfonamide (80 mg) as a brown oil. LCMS (ESI) m/z: 490 [M+H]⁺.

Step 4: A solution of 1-[(2-aminoethoxy)methyl]-N-(1-methylcyclopropyl)-4-[(1-methylpyrazol-4-yl)(2H2)methyl]-5-oxo-1H,2H-imidazo[1,2-a]quinazoline-7-sulfonamide (60 mg, 0.12 mmol) in MeOH (5 mL) was treated with sodium cyanoborohydride (15.4 mg, 0.25 mmol) at 0° C. for 30 min under nitrogen atmosphere followed by the addition of formaldehyde (7.4 mg, 0.25 mmol) at 0° C. The reaction mixture was stirred at room temperature for a period of 90 min. After completion of reaction, the reaction mixture was quenched by addition of water (1 mL). The aqueous layer was extracted with ethyl acetate (50 mL). The combined organic phase was washed with brine (20 mL), dried over anhydrous sodium sulfate and concentrated under reduced pressure. The residue was purified by reversed-phase flash chromatography with the following conditions: column, C18 silica gel; mobile phase, MeCN in Water (0.1% NH₃·H₂O+10 mmol/L NH₄HCO₃), 10% to 60% gradient in 300 min; detector, UV 254 nm. This resulted in 1-{[2-(dimethylamino)ethoxy]methyl}-N-(1-methylcyclopropyl)-4-[(1-methylpyrazol-4-yl)(2H2)methyl]-5-oxo-1H,2H-imidazo[1,2-a]quinazoline-7-sulfonamide (20 mg) as a white solid. LCMS (ESI) m/z: 518 [M+H]⁺.

Step 5: The racemate (20 mg) was purified by Prep-CHIRAL-HPLC with the following conditions; Column: JW-CHIRAL ART Cellulose-SZ, 3.0*50 mm; 3 um; Mobile Phase A: Hex (10 mM NH₃-MeOH), Mobile Phase B: EtOH; Flow rate: 40 mL/min; Gradient: isocratic 50) to afford (1S)-1-{[2-(dimethylamino)ethoxy]methyl}-N-(1-methylcyclopropyl)-4-[(1-methylpyrazol-4-yl)(2H2)methyl]-5-oxo-1H,2H-imidazo[1,2-a]quinazoline-7-sulfonamide (5.1 mg, 26%) and (1R)-1-{[2-(dimethylamino)ethoxy]methyl}-N-(1-methylcyclopropyl)-4-[(1-methylpyrazol-4-yl)(2H2)methyl]-5-oxo-1H,2H-imidazo[1,2-a]quinazoline-7-sulfonamide (5.1 mg, 26%) both as white solids. LCMS (ESI) m/z: 518.30 [M+H]⁺. ¹H NMR (400 MHz, Methanol-d₄) δ8.44-8.35 (m, 1H), 8.02-7.95 (m, 1H), 7.68 (s, 1H), 7.59-7.53 (m, 1H), 7.35-7.27 (m, 1H), 4.82 (s, 1H), 4.15-4.07 (m, 1H), 3.94-3.80 (m, 5H), 3.61-3.52 (m, 2H), 3.53-3.47 (m, 1H), 2.46-2.37 (m, 2H), 2.15-2.05 (m, 6H), 1.16 (s, 3H), 0.75-0.64 (m, 2H), 0.48-0.37 (m, 2H).

Representative Synthesis Route 27: Example 128: (R)-1-(2-cyanoethyl)-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

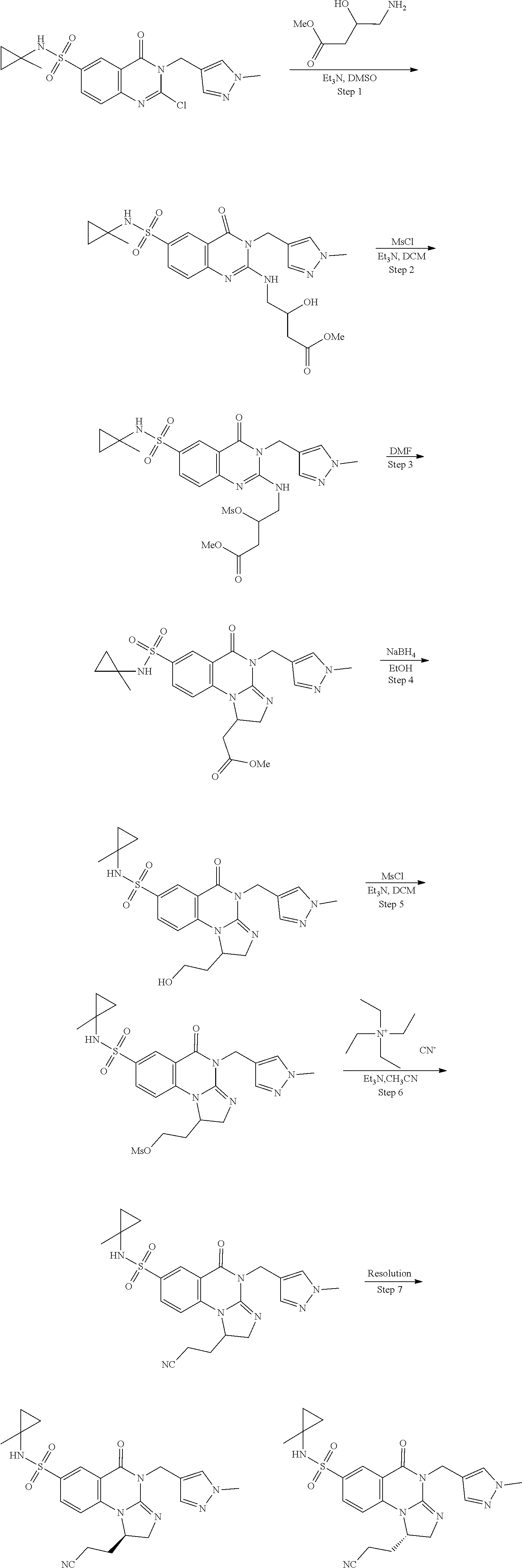

Step 1: To a stirred mixture of methyl 4-amino-3-hydroxybutanoate (600 mg, 4.51 mmol) and 2-chloro-N-(1-methylcyclopropyl)-3-[(1-methylpyrazol-4-yl)methyl]-4-oxoquinazoline-6-sulfonamide (459.5 mg, 1.13 mmol) in DMSO (10 mL) were added Et₃N (229 mg, 2.26 mmol) dropwise at room temperature under nitrogen atmosphere. The reaction was quenched by the addition of water (50 mL) at room temperature. The resulting mixture was extracted with EtOAc (2×50 mL). The combined organic layers were washed with brine (50 mL), dried over anhydrous Na₂SO₄. After filtration, the filtrate was concentrated under reduced pressure. The residue was purified by silica gel column chromatography, eluted with MeOH/CH₂Cl₂ (0-10% o) to afford methyl 3-hydroxy-4-({6-[(1-methyl cyclopropyl)sulfamoyl]-3-[(1-methylpyrazol-4-yl)methyl]-4-oxoquinazolin-2-yl}amino)butanoate (500 mg) as a yellow oil. LCMS (ESI) m/z: 505 [M+H]⁺ Step 2: To a stirred mixture of methyl 3-hydroxy-4-({6-[(1-methylcyclopropyl)sulfamoyl]-3-[(1-methylpyrazol-4-yl)methyl]-4-oxoquinazolin-2-yl}amino)butanoate (500 mg, 0.99 mmol) and Et₃N (1.00 g, 9.91 mmol) in DCM (20 mL) were added methanesulfonyl chloride (1.14 g, 9.91 mmol) dropwise at room temperature. The resulting mixture was stirred at room temperature for 4 h. The resulting mixture was concentrated under reduced pressure to obtain methyl 3-(methanesulfonyloxy)-4-{[(2E)-6-[(1-methylcyclopropyl)sulfamoyl]-3-[(1-methylpyrazol-4-yl)methyl]-4-oxo-1H-quinazolin-2-ylidene]amino}butanoate (450 mg) as a yellow oil, which was used in the next step directly without further purification. LCMS (ESI) m/z: 583 [M+H]⁺.

Step 3: A mixture of methyl 3-hydroxy-4-({6-[(1-methylcyclopropyl)sulfamoyl]-3-[(1-methylpyrazol-4-yl)methyl]-4-oxoquinazolin-2-yl}amino)butanoate (450 mg, 0.89 mmol) in DMF (10 mL) was stirred at 50° C. for 16 h. The reaction was quenched by the addition of water (100 mL) at room temperature. The resulting mixture was extracted with EtOAc (2×100 mL). The combined organic layers were washed with brine (100 mL), dried over anhydrous Na₂SO₄. After filtration, the filtrate was concentrated under reduced pressure. The residue was purified by silica gel column chromatography, eluted with MeOH/CH₂Cl₂ (0-10%) to afford methyl 2-{7-[(1-methylcyclopropyl)sulfamoyl]-4-[(1-methylpyrazol-4-yl)methyl]-5-oxo-1H,2H-imidazo[1,2-a]quinazolin-1-yl}acetate (350 mg) as a yellow solid. LCMS (ESI) m/z: 487 [M+H]⁺.

Step 4: A mixture of methyl 2-{7-[(1-methylcyclopropyl)sulfamoyl]-4-[(1-methylpyrazol-4-yl)methyl]-5-oxo-1H,2H-imidazo[1,2-a]quinazolin-1-yl}acetate (350 mg, 0.72 mmol) and NaBH₄ (54.4 mg, 1.44 mmol) in EtOH (10 mL) was stirred at room temperature for 2 h. The reaction was quenched by the addition of water (50 mL) at room temperature. The resulting mixture was extracted with EtOAc (2×50 mL). The combined organic layers were washed with brine (50 mL), dried over anhydrous Na₂SO₄. After filtration, the filtrate was concentrated under reduced pressure. The residue was purified by silica gel column chromatography, eluted with MeOH/CH₂Cl₂ (0-10%) to afford 1-(2-hydroxyethyl)-N-(1-methylcyclopropyl)-4-[(1-methylpyrazol-4-yl)methyl]-5-oxo-1H,2H-imidazo[1,2-a]quinazoline-7-sulfonamide (250 mg) as a yellow oil. LCMS (ESI) m/z: 459 [M+H]⁺.

Step 5: To a stirred mixture of 1-(2-hydroxyethyl)-N-(1-methylcyclopropyl)-4-[(1-methylpyrazol-4-yl)methyl]-5-oxo-1H,2H-imidazo[1,2-a]quinazoline-7-sulfonamide (250 mg, 0.54 mmol) and Et₃N (551.7 mg, 5.45 mmol) in DCM (10 mL) were added methanesulfonyl chloride (624.5 mg, 5.45 mmol) dropwise at room temperature. The resulting mixture was stirred at room temperature for 16 h. The resulting mixture was concentrated under reduced pressure, residue dissolved in H₂O (50 mL) and extracted with EtOAc (2×50 mL). The combined organic layers were washed with brine (50 mL), dried over anhydrous Na₂SO₄. After filtration, the filtrate was concentrated under reduced pressure. The residue was purified by silica gel column chromatography, eluted with MeOH/CH₂Cl₂ (0-10%) to afford 2-{7-[(1-methylcyclopropyl)sulfamoyl]-4-[(1-methylpyrazol-4-yl)methyl]-5-oxo-1H,2H-imidazo[1,2-a]quinazolin-1-yl}ethyl methanesulfonate (200 mg) as a white solid. LCMS (ESI) m/z: 537 [M+H]⁺.

Step 6: To a stirred mixture of 2-{7-[(1-methylcyclopropyl)sulfamoyl]-4-[(1-methylpyrazol-4-yl)methyl]-5-oxo-1H,2H-imidazo[1,2-a]quinazolin-1-yl}ethyl methanesulfonate (400 mg, 0.74 mmol) and Et₃N (226.3 mg, 2.23 mmol) in CH₃CN (10 mL) were added tetraethyl ammonium cyanide (232.9 mg, 1.49 mmol) in portions at room temperature. The resulting mixture was stirred at 70° C. for 8 h. The reaction was quenched by the addition of water (100 mL) at room temperature. The resulting mixture was extracted with EtOAc (2×100 mL). The combined organic layers were washed with brine (100 mL), dried over anhydrous Na₂SO₄. After filtration, the filtrate was concentrated under reduced pressure. The residue was purified by silica gel column chromatography, eluted with CH₂Cl₂/MeOH (1:4) to afford 1-(2-cyanoethyl)-N-(1-methylcyclopropyl)-4-[(1-methylpyrazol-4-yl)methyl]-5-oxo-1H,2H-imidazo[1,2-a]quinazoline-7-sulfonamide (200 mg, 57% yield, 50% purity) as a yellow solid. LCMS (ESI) m/z: 468 [M+H]

Step 7: The racemate (100 mg) was purified by Prep-Chiral-HPLC with the following conditions (Column: CHIRALPAK-IK, 3*25 mm, 5 m; Mobile Phase A: Hex (10 mM NH₃-MeOH), Mobile Phase B: EtOH; Flow rate: 40 mL/min; Gradient: isocratic 40; Wave Length: 202/220 nm; Sample Solvent: EtOH: DCM=1:1) to afford (1R)-1-(2-cyanoethyl)-N-(1-methylcyclopropyl)-4-[(1-methylpyrazol-4-yl)methyl]-5-oxo-1H,2H-imidazo[1,2-a]quinazoline-7-sulfonamide (50 mg, 25%), (1S)-1-(2-cyanoethyl)-N-(1-methylcyclopropyl)-4-[(1-methylpyrazol-4-yl)methyl]-5-oxo-1H,2H-imidazo[1,2-a]quinazoline-7-sulfonamide (2.2 mg, 1%), both as white solids. LCMS (ESI) m/z: 468.10 [M+H]⁺. 1H NMR (400 MHz, DMSO-d₆) δ 8.26 (s, 1H), 8.07 (s, 1H), 7.94 (d, J=8.6 Hz, 1H), 7.69 (s, 1H), 7.42 (s, 1H), 7.32 (s, 1H), 5.02-4.84 (m, 2H), 4.77 (s, 1H), 4.10-3.95 (m, 1H), 3.76 (s, 4H), 2.60 (d, J=6.8 Hz, 2H), 2.04 (d, J=14.6 Hz, 2H), 1.07 (s, 3H), 0.59 (t, J=3.2 Hz, 2H), 0.38 (dd, J=4.9, 2.2 Hz, 2H).

Representative Synthesis Route 28: Example 132: 4-((1-methyl-1H-pyrazol-4-yl)methyl)-N-(1-methylcyclopropyl)-5-oxo-1-(pyrimidin-4-ylethynyl)-1,2,4,5-tetrahydroimidazo[1,2-a]quinazoline-7-sulfonamide

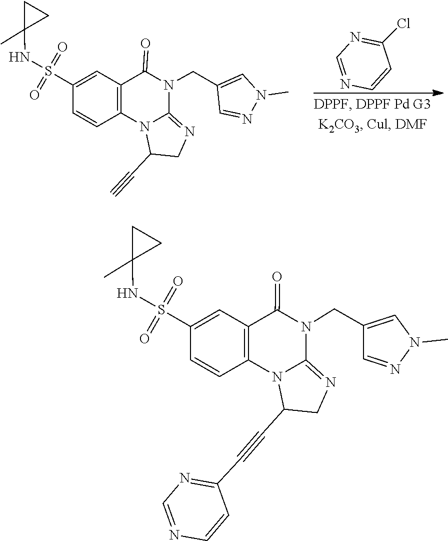

To a stirred solution of 1-ethynyl-N-(1-methylcyclopropyl)-4-[(1-methylpyrazol-4-yl)methyl]-5-oxo-1H,2H-imidazo[1,2-a]quinazoline-7-sulfonamide (100 mg, 0.22 mmol) and 4-chloro pyrimidine (20.9 mg, 0.18 mmol) in DMF (10 mL) were added K₂CO₃ (94.5 mg, 0.68 mmol), CuI (8.69 mg, 0.04 mmol) and DPPF Pd G3 (21.3 mg, 0.02 mmol), DPPF (12.6 mg, 0.02 mmol) at room temperature. The resulting mixture was stirred at 90° C. for 16 h under nitrogen atmosphere. The reaction was quenched by the addition of water (30 mL) at room temperature. The resulting mixture was extracted with EtOAc (3×20 mL). The combined organic layers were washed with brine (2×20 mL), dried over anhydrous Na₂SO₄. After filtration, the filtrate was concentrated under reduced pressure. The crude product (80 mg) was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min mL/min; Gradient: 5% B to 5% B in 1.5 min, 5% B to 21% B in 2 min, 21% to 38% B in 10 min; Wave Length: 254 nm/220 nm) to afford 4-((1-methyl-1H-pyrazol-4-yl)methyl)-N-(1-methylcyclopropyl)-5-oxo-1-(pyrimidin-4-ylethynyl)-1,2,4,5-tetrahydroimidazo[1,2-a]quinazoline-7-sulfonamide (40.0 mg, 34%) as an off-white solid. LCMS (ESI) m/z: 517.25 [M+H]⁺. ¹H NMR (400 MHz, Methanol-d₄) δ 9.08 (t, J=2.0 Hz, 1H), 8.76 (dd, J=5.2, 2.4 Hz, 1H), 8.44 (q, J=2.4 Hz, 1H), 8.04 (dt, J=8.8, 2.4 Hz, 1H), 7.70 (s, 1H), 7.59-7.50 (m, 2H), 7.38 (dd, J=8.8, 3.2 Hz, 1H), 5.65 (dd, J=10.4, 5.2 Hz, 1H), 5.03 (d, J=3.2 Hz, 2H), 4.46 (dd, J=13.6, 10.4 Hz, 1H), 4.22 (dd, J=13.6, 5.2 Hz, 1H), 3.82 (d, J=2.0 Hz, 3H), 1.14 (d, J=2.0 Hz, 3H), 0.69 (q, J=4.4 Hz, 2H), 0.45-0.37 (m, 2H).

The following compounds in Table 17 were prepared using procedures similar to those described in Representative Synthesis Route 28 for Example 132 using appropriate starting materials.

TABLE 17

| Example | | |
|---|---|---|
| No. | Name | [M + H]⁺ |
| 133 | 4-methyl-1-((1-methyl-1H-indazol-6-yl)ethynyl)-N-(1- | 489.10 |
| | methylcyclopropyl)-5-oxo-1,2,4,5-tetrahydroimidazo[1,2- | |
| | a]quinazoline-7-sulfonamide | |
| 142 | (R)-4-methyl-1-((2-methyl-2H-indazol-5-yl)ethynyl)-N-(1- | 489.15 |
| | methylcyclopropyl)-5-oxo-1,2,4,5-tetrahydroimidazo[1,2- | |
| | a]quinazoline-7-sulfonamide | |

Representative Synthesis Route 29: Example 134: 1-((5-cyanopyridin-3-yl)ethynyl)-4-methyl-N-(1-methylcyclopropyl)-5-oxo-1,2,4,5-tetrahydroimidazo[1,2-a]quinazoline-7-sulfonamide

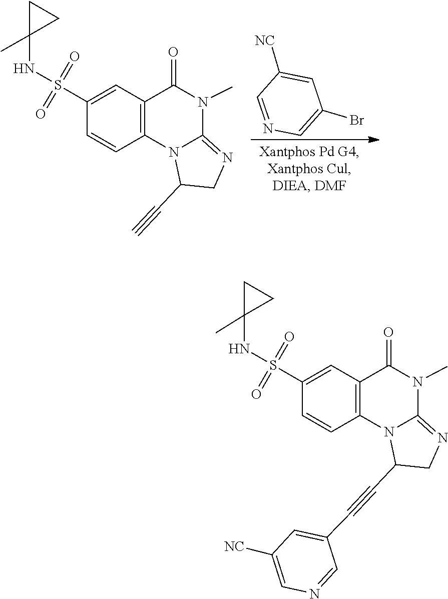

To a stirred solution of 1-ethynyl-4-methyl-N-(1-methylcyclopropyl)-5-oxo-1H,2H-imidazo[1,2-a]quinazoline-7-sulfonamide (100 mg, 0.27 mmol) and 5-bromopyridine-3-carbonitrile (40.8 mg, 0.22 mmol) in DMF (8 mL) were added XantPhos Pd G4 (26.8 mg, 0.02 mmol), XantPhos (16.1 mg, 0.02 mmol) and DIEA (115.7 mg, 0.83 mmol), CuI (10.6 mg, 0.05 mmol) at room temperature. The resulting mixture was stirred at 90° C. for 2 h under nitrogen atmosphere. The reaction was quenched by the addition of water (100 mL) at room temperature. The resulting mixture was extracted with EtOAc (3×50 mL). The combined organic layers were washed with brine (2×50 mL), dried over anhydrous Na₂SO₄. The crude product (60 mg) was purified by Prep-HPLC with the following conditions (Column: XBridge Shield RP18 OBD Column 30*150 mm; Mobile Phase A: Water (10 mmol/L NH₄HCO₃+0.05% NH₃·H₂O), Mobile Phase B: ACN; Flow rate: 60 mL/min mL/min; Gradient: 21% B to 37% B in 10 min; Wave Length: 254 nm/220 nm; RT1 (min): 13.7) to afford 1-((5-cyanopyridin-3-yl)ethynyl)-4-methyl-N-(1-methylcyclopropyl)-5-oxo-1,2,4,5-tetrahydroimidazo[1,2-a]quinazoline-7-sulfonamide (23.8 mg, 18%), as a white solid. LCMS (ESI) m/z: 461.10 [M+H]⁺. 1H NMR (400 MHz, Methanol-d₄) δ 8.83 (dd, J=15.6, 2.0 Hz, 2H), 8.45 (d, J=2.0 Hz, 1H), 8.24 (t, J=2.0 Hz, 1H), 8.07 (dd, J=8.8, 2.0 Hz, 1H), 7.43 (d, J=8.8 Hz, 1H), 5.65 (dd, J=10.2, 4.8 Hz, 1H), 4.41 (dd, J=13.6, 10.4 Hz, 1H), 4.17 (dd, J=13.6, 4.8 Hz, 1H), 3.44 (s, 3H), 1.15 (s, 3H), 0.74-0.66 (m, 2H), 0.46-0.39 (m, 2H).

The following compounds in Table 18 were prepared using procedures similar to those described in Representative Synthesis Route 29 for Example 134 using appropriate starting materials.

TABLE 18

| Example | | |
|---|---|---|
| No. | Name | [M + H]⁺ |
| 193 | (R)-1-((1-cyclopropyl-1H-pyrazol-5-yl)ethynyl)-4-((1-methyl- | 545.25 |
| | 1H-pyrazol-4-yl)methyl)-N-(1-methylcyclopropyl)-5-oxo- | |
| | 1,2,4,5-tetrahydroimidazo[1,2-a]quinazoline-7-sulfonamide | |
| 196 | (R)-1-((1-methyl-1H-1,2,4-triazol-5-yl)ethynyl)-4-((1-methyl- | 520.15 |
| | 1H-pyrazol-4-yl)methyl)-N-(1-methylcyclopropyl)-5-oxo- | |
| | 1,2,4,5-tetrahydroimidazo[1,2-a]quinazoline-7-sulfonamide | |
| 206 | (R)-1-((7-cyanoimidazo[1,2-b]pyridazin-3-yl)ethynyl)-4-ethyl- | 515.05 |
| | N-(1-methylcyclopropyl)-5-oxo-1,2,4,5- | |
| | tetrahydroimidazo[1,2-a]quinazoline-7-sulfonamide | |
| 217 | (R)-1-((4-fluoro-1-methyl-1H-pyrazol-5-yl)ethynyl)-4-((1- | 537.15 |
| | methyl-1H-pyrazol-4-yl)methyl)-N-(1-methylcyclopropyl)-5- | |
| | oxo-1,2,4,5-tetrahydroimidazo[1,2-a]quinazoline-7- | |
| | sulfonamide | |

Representative Synthesis Route 30: Example 139: (R)-4-(2,2-difluoroethyl)-1-methyl-N-(1-methylcyclopropyl)-5-oxo-1,2,4,5-tetrahydroimidazo[1,2-a]quinazoline-7-sulfonamide

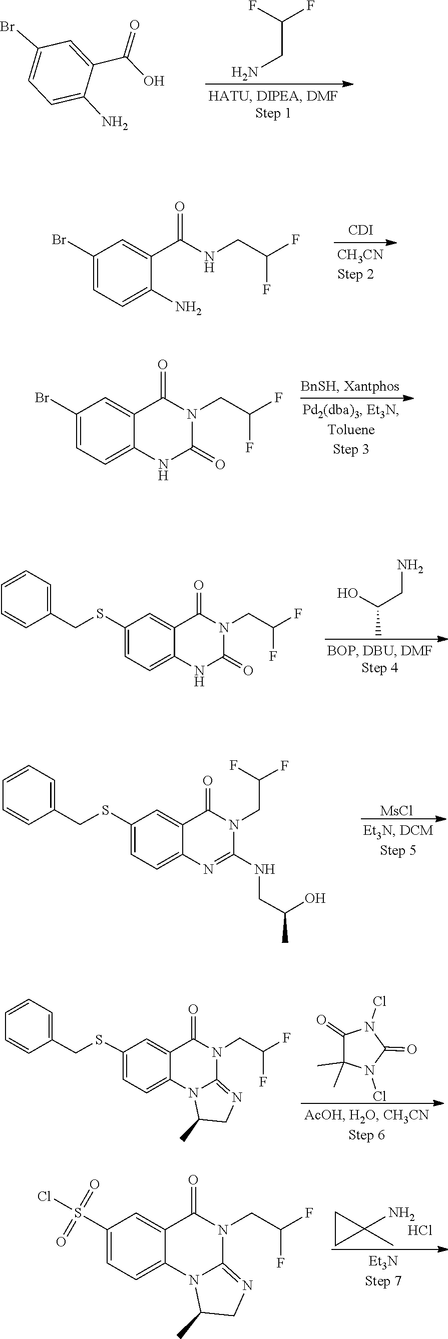

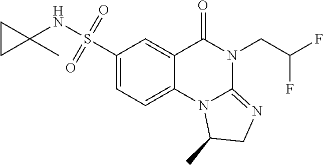 [SMILES: C[C@@H]1CN=C2N(CC(F)F)C(=O)c3cc(S(=O)(=O)NC4(C)CC4)ccc3N21]

Step 1: To a solution of 2-amino-5-bromobenzoic acid (3 g, 13.9 mmol) in DMF (77 mL) added HATU (5.8 g, 15.3 mmol), DIPEA (3.4 mL, 19.4 mmol) and 2,2-difluoroethylamine (0.98 mL, 13.9 mmol). Reaction was stirred at rt for 1.45 h. To the reaction mixture added water and extracted with EtOAc, dried over anhydrous Na₂SO₄, evaporated solvent in vacuo. To the crude reaction mixture water was added dropwise and the formed precipitate was filtered and dried to obtain a pale yellow solid (3.4 g, 88%). LCMS (ESI) m/z: 279, 281 [M+H]⁺.

Step 2: To a solution of N-2,2-difluoroethyl 2-amino-5-bromobenzamide (2 g, 7.17 mmol) in CH₃CN (33 mL) added CDI (2.1 g, 12.9 mmol) and DMAP (87.6 mg, 0.72 mmol). The reaction was stirred at rt for 25 min, 55° C. (sand bath) for 1.40 h and then at 80° C. for 2.10 h. Don't observe any product formation. To this reaction mixture added DIPEA (2.2 mL) dropwise and heated at 80° C. overnight. Don't observe any product formation. Additional CDI (1.4g) and DIPEA (4 mL) was added and heated for another 1.5 h. Don't observe any product formation. Reaction mixture was transferred into two 40 mL vials and heated in a heat block at 75° C. for 2 h. Desired product was observed by LCMS. The reaction was concentrated in vacuo to obtain a yellow solid. To the residue added water and the formed ppt was filtered and washed with 1M HCl. The solid was dried to obtain a pale yellow solid (2.04 g, 93%). LCMS (ESI) m/z: 305, 307 [M+H]⁺.

Step 3: To a stirred solution of 6-bromo-3-(2,2-difluoroethyl)-1,2,3,4-tetrahydro-2,4-quinazolinedione (0.6 g, 1.97 mmol) in toluene (11.4 mL) added Et₃N (0.8 mL, 5.9 mmol), Xantphos (228 mg, 0.39 mmol) and Pd₂(dba)₃ (180 mg, 0.19 mmol). The reaction mixture was degassed and back filled with N₂, twice. Subsequently added BnSH (0.27 mL, 2.36 mmol) and the reaction mixture was heated at 90° C. in a heat block for 1.15 h. Crude reaction mixture was purified by silica gel chromatography (0-130% MeOH/DCM) to obtain product as a pale yellow solid (707 mg, 94%). LCMS (ESI) m/z: 349 [M+H]⁺.

Step 4: A mixture of 6-(benzylthio)-3-(2,2-difluoroethyl)-2,4(1H,3H)-quinazolinedione (415 mg, 1.19 mmol), DMF (0.5 mL), BOP (1.52 g, 3.4 mmol), DBU (0.51 mL, 3.4 mmol) was stirred at rt for 1.5 h. To this solution added (S)-1-amino-2-propanol (0.44 mL, 5.7 mmol) and reacted at rt for 30 min. The reaction was quenched by the addition of water at room temperature. The resulting mixture was extracted with EtOAc. The combined organic layers were washed with brine, dried over anhydrous Na₂SO₄. After filtration, the filtrate was concentrated under reduced pressure. Crude reaction mixture was purified by silica gel chromatography (0-15% MeOH/DCM) to obtain product as an orange oily solid (417 mg, 86%). LCMS (ESI) m/z: 406 [M+H]⁺.

Step 5: To a solution of 2-[(S)-2-hydroxypropylamino]-6-(benzylthio)-3-(2,2-difluoroethyl)-4(3H)-quinazolinone (468 mg, 1.15 mmol) in DCM (23.8 mL) at room temperature added Et₃N (1.4 mL, 10.3 mmol) and MsCl (0.22 mL, 2.89 mmol) dropwise. Reaction was stirred at rt for 3 h and then heated at 38° C. for 1 h. To the reaction mixture water was added and extracted with DCM and EtOAc. The combined organic layers were washed with brine, dried over anhydrous Na₂SO₄. After filtration, the filtrate was concentrated under reduced pressure. Crude reaction mixture was purified by silica gel chromatography (0-15% MeOH/DCM) to obtain product as an orange oily solid, 447 mg (75% purity) which was used for the next step without further purification. LCMS (ESI) m/z: 388 [M+H]⁺.

Step 6: A mixture of (R)-7-(benzylthio)-4-(2,2-difluoroethyl)-1-methyl-2,4-dihydroimidazo[1,2-a]quinazolin-5(1H)-one (63.9 mg, 0.16 mmol) in CH₃CN (0.65 mL) was cooled to 0° C. and added of H₂O (1 drop), AcOH (2 drops), 1,3-dichloro-5,5-dimethylimidazolidine-2,4-dione (35.7 mg, 0.18 mmol). The reaction was stirred at 0° C. for 1 h. Additional 1,3-dichloro-5,5-dimethyl-2,4-imidazolidinedione (17.2 mg) was added at 0° C. and stirred at 0° C. for another 30 min. Observe complete conversion to product. Used the reaction mixture directly for the next step. LCMS (ESI) m/z: 364 [M+H]⁺.

Step 7: The reaction mixture from the previous step was cooled to 0° C. and added 1-methylcyclopropan-1-amine hydrochloride (47.3 mg, 0.44 mmol) and Et₃N (0.32 mL, 2.3 mmol). The reaction was stirred at 0° C. for 30 min and then at rt for 50 min. Reaction mixture was concentrated, added water and extracted with EtOAc. Organic layer was dried and purified by silica gel chromatography (0-2% MeOH/DCM) and then (0-50% EtOAc/hex) to obtain product as a white solid (15.7 mg, 29%). LCMS (ESI) m/z: 399.00 [M+H]⁺. ¹H NMR (400 MHz, DMSO-d₆) δ 8.25 (s, 1H), 8.07 (s, 1H), 7.80 (d, J=8.0 Hz, 1H), 7.28 (d, J=8.0 Hz, 1H), 6.44-6.20 (m, 1H), 4.76-4.72 (m, 1H), 4.38-4.30 (m, 2H), 4.04-3.99 (m, 1H), 3.50-3.47 (m, 1H), 1.33 (d, J=6.0 Hz, 3H), 1.06 (s, 3H), 0.61-0.58 (m, 2H), 0.44-0.37 (m, 2H).

Representative Synthesis Route 31: Example 140: (R)-4-((1-methyl-1H-pyrazol-4-yl)methyl)-1-((2-methyl-2H-1,2,3-triazol-4-yl)ethynyl)-N-(1-methylcyclopropyl)-5-oxo-1,2,4,5-tetrahydroimidazo[1,2-a]quinazoline-7-sulfonamide

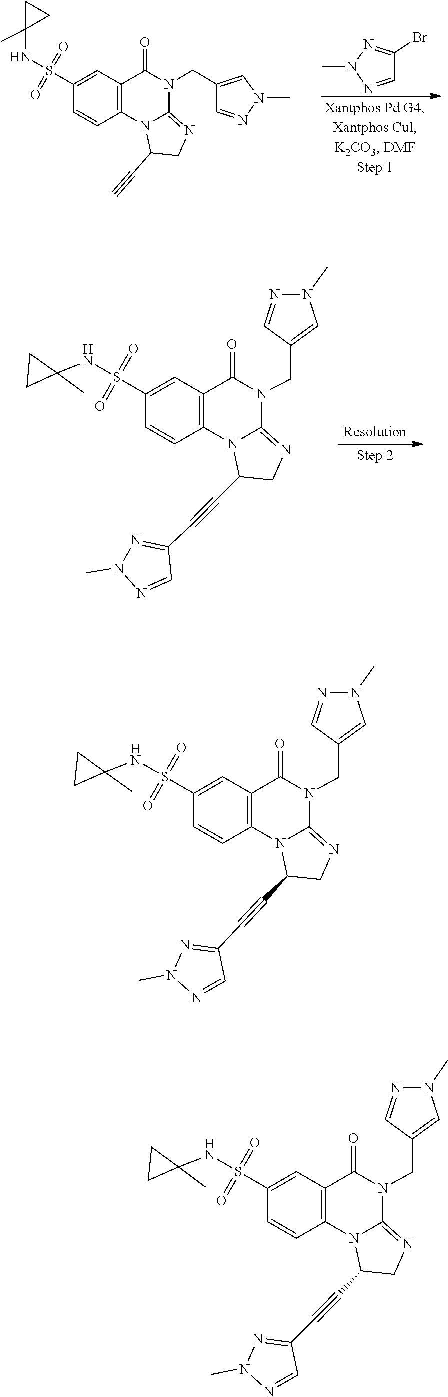

Step 1: To a stirred solution of 1-ethynyl-N-(1-methylcyclopropyl)-4-[(1-methylpyrazol-4-yl)methyl]-5-oxo-1H,2H-imidazo[1,2-a]quinazoline-7-sulfonamide (100 mg, 0.22 mmol) and 4-bromo-2-methyl-1,2,3-triazole (29.5 mg, 0.18 mmol) in DMF (10 mL) were added K₂CO₃ (94.5 mg, 0.68 mmol), CuI (8.7 mg, 0.04 mmol) and XantPhos Pd G4 (21.9 mg, 0.02 mmol), XantPhos (13.2 mg, 0.02 mmol) at room temperature. The resulting mixture was stirred at 90° C. for 16 h under nitrogen atmosphere. The reaction was quenched by the addition of water (30 mL) at room temperature. The resulting mixture was extracted with EtOAc (3×20 mL). The combined organic layers were washed with brine (2×20 mL), dried over anhydrous Na₂SO₄. After filtration, the filtrate was concentrated under reduced pressure. The crude product (80 mg) was purified by Prep-HPLC with the following conditions (Column: Xselect CSH C18 OBD Column 30*150 mm 5 m; Mobile Phase A: Water (0.1% FA), Mobile Phase B: ACN; Flow rate: 60 mL/min; Gradient: 5% B to 5% B in 1.5 min, 5% B to 27% B in 2 min, 27% to 45% B in 10 min; Wave Length: 254 nm/220 nm; RT1 (min): 8.37) to afford 1-[2-(2-methyl-1,2,3-triazol-4-yl)ethynyl]-N-(1-methylcyclopropyl)-4-[(1-methylpyrazol-4-yl)methyl]-5-oxo-1H,2H-imidazo[1,2-a]quinazoline-7-sulfonamide (27.6 mg, 23%) as a white solid. LCMS (ESI) m/z: 520 [M+H]⁺.

Step 2: The racemate (24.6 mg) was purified by chiral-Prep-HPLC with the following conditions (Mobile Phase A: MTBE (0.1% DEA): EtOH=80:20; Flow rate: 1.0 ml/min; Gradient: isocratic) to afford (1R)-1-[2-(2-methyl-1,2,3-triazol-4-yl)ethynyl]-N-(1-methylcyclopropyl)-4-[(1-methylpyrazol-4-yl)methyl]-5-oxo-1H,2H-imidazo[1,2-a]quinazoline-7-sulfonamide (8.1 mg, 34%) and (1 S)-1-[2-(2-methyl-1,2,3-triazol-4-yl)ethynyl]-N-(1-methylcyclopropyl)-4-[(1-methylpyrazol-4-yl)methyl]-5-oxo-1H,2H-imidazo[1,2-a]quinazoline-7-sulfonamide (8.1 mg, 34%) both as off-white solids. LCMS (ESI) m/z: 520.15 [M+H]⁺. 1H NMR (400 MHz, Methanol-d₄) δ 8.46 (d, J=2.0 Hz, 1H), 8.08-8.01 (m, 1H), 7.71 (d, J=10.8 Hz, 2H), 7.57 (d, J=0.8 Hz, 1H), 7.38 (d, J=8.8 Hz, 1H), 5.58 (dd, J=10.4, 5.2 Hz, 1H), 5.03 (s, 2H), 4.42 (dd, J=13.6, 10.4 Hz, 1H), 4.20-4.10 (m, 4H), 3.83 (s, 3H), 1.15 (s, 3H), 0.74-0.66 (m, 2H), 0.46-0.38 (m, 2H).

The following compounds in Table 19 were prepared using procedures similar to those described in Representative Synthesis Route 31 using appropriate starting materials.

TABLE 19

| Example | | |
|---|---|---|
| No. | Name | [M + H]⁺ |
| 147 | (R)-4-methyl-1-((2-methyl-2H-indazol-6-yl)ethynyl)-N-(1- | 489.15 |
| | methylcyclopropyl)-5-oxo-1,2,4,5-tetrahydroimidazo[1,2- | |
| | a]quinazoline-7-sulfonamide | |
| 148 | (R)-4-methyl-1-((1-methyl-1H-indazol-5-yl)ethynyl)-N-(1- | 489.15 |
| | methylcyclopropyl)-5-oxo-1,2,4,5-tetrahydroimidazo[1,2- | |
| | a]quinazoline-7-sulfonamide | |
| 149 | (R)-1-(imidazo[1,2-b]pyridazin-3-ylethynyl)-4-methyl-N-(1- | 476.15 |
| | methylcyclopropyl)-5-oxo-1,2,4,5-tetrahydroimidazo[1,2- | |
| | a]quinazoline-7-sulfonamide | |
| 172 | 4-methyl-1-((1-methyl-1H-benzo[d][1,2,3]triazol-6- | 490.20 |
| | yl)ethynyl)-N-(1-methylcyclopropyl)-5-oxo-1,2,4,5- | |
| | tetrahydroimidazo[1,2-a]quinazoline-7-sulfonamide | |
| 179 | 1-(isothiazol-4-ylethynyl)-4-methyl-N-(1-methylcyclopropyl)- | 442.00 |
| | 5-oxo-1,2,4,5-tetrahydroimidazo[1,2-a]quinazoline-7- | |
| | sulfonamide | |
| 189 | (R)-4-ethyl-1-(imidazo[1,2-b]pyridazin-3-ylethynyl)-N-(1- | 490.00 |
| | methylcyclopropyl)-5-oxo-1,2,4,5-tetrahydroimidazo[1,2- | |
| | a]quinazoline-7-sulfonamide | |
| 199 | (R)-4-methyl-1-((1-methyl-6-oxo-1,6-dihydropyridazin-4- | 467.10 |
| | yl)ethynyl)-N-(1-methylcyclopropyl)-5-oxo-1,2,4,5- | |
| | tetrahydroimidazo[1,2-a]quinazoline-7-sulfonamide | |
| 221 | (R)-1-(imidazo[1,2-b]pyridazin-3-ylethynyl)-4-((1-methyl-1H- | 556.20 |
| | pyrazol-4-yl)methyl)-N-(1-methylcyclopropyl)-5-oxo-1,2,4,5- | |
| | tetrahydroimidazo[1,2-a]quinazoline-7-sulfonamide | |
| 231 | (R)-1-((1,3-dimethyl-1H-pyrazol-5-yl)ethynyl)-4-((1-methyl- | 535.15 |
| | 1H-pyrazol-4-yl)methyl-d2)-N-(1-methylcyclopropyl)-5-oxo- | |
| | 1,2,4,5-tetrahydroimidazo[1,2-a]quinazoline-7-sulfonamide | |

Representative Synthesis Route 32: Example 146: (R)-9-(3-cyano-3-methylazetidin-1-yl)-1-methyl-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

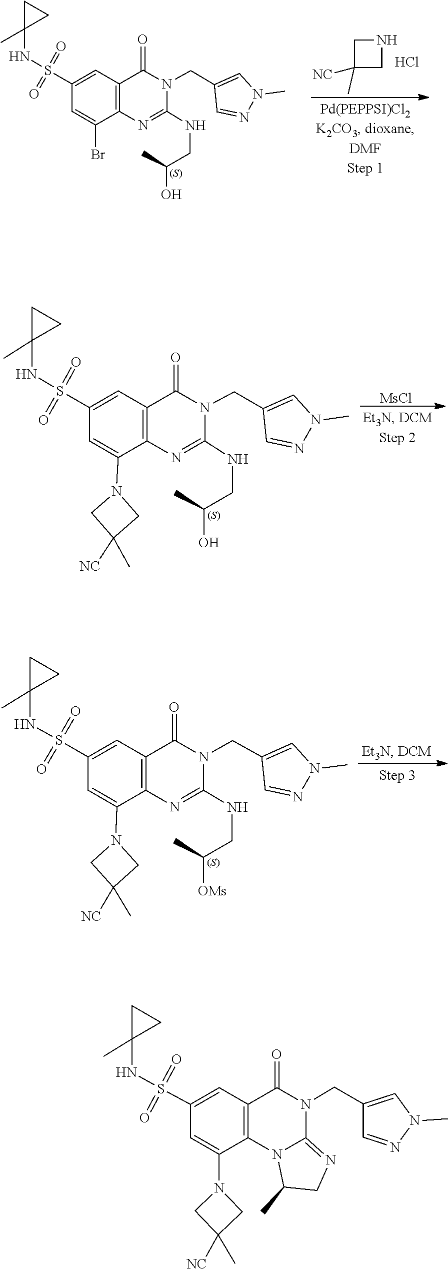

Step 1: A solution of (S)-8-bromo-2-((2-hydroxypropyl)amino)-3-((1-methyl-1H-pyrazol-4-yl)methyl)-N-(1-methylcyclopropyl)-4-oxo-3,4-dihydroquinazoline-6-sulfonamide (200 mg, 0.38 mmol), 3-methylazetidine-3-carbonitrile hydrochloride (60.6 mg, 0.46 mmol), K₂CO₃(210.4 mg, 1.52 mmol,) and (SP-4-1)-[1,3-Bis[2,6-bis(1-ethylpropyl)phenyl]-4,5-dichloro-1,3-dihydro-2H-imidazol-2-ylidene]dichloro(2-methylpyridine)palladium (31.9 mg, 0.038 mmol) in 1,4-dioxane (4 mL) and DMF (0.4 mL) was stirred at 100° C. for 1 h under nitrogen atmosphere. The reaction was quenched by the addition of water (50 mL) at room temperature. The resulting mixture was extracted with EtOAc (2×50 mL). The combined organic layers were washed with brine (10 mL), dried over anhydrous Na₂SO₄. After filtration, the filtrate was concentrated under reduced pressure. The residue was purified by silica gel column chromatography, eluted with CH₂Cl₂/MeOH (9:1) to afford (S)-8-(3-cyano-3-methylazetidin-1-yl)-2-((2-hydroxypropyl)amino)-3-((1-methyl-1H-pyrazol-4-yl)methyl)-N-(1-methylcyclopropyl)-4-oxo-3,4-dihydroquinazoline-6-sulfonamide (100 mg, 48%) as a yellow solid. LCMS (ESI) m/z: 541 [M+H]⁺.

Step 2: A solution of (S)-8-(3-cyano-3-methylazetidin-1-yl)-2-((2-hydroxypropyl)amino)-3-((1-methyl-1H-pyrazol-4-yl)methyl)-N-(1-methylcyclopropyl)-4-oxo-3,4-dihydroquinazoline-6-sulfonamide (80 mg, 0.15 mmol), methanesulfonyl chloride (33.9 mg, 0.29 mmol) and Et₃N (44.9 mg, 0.44 mmol) in DCM (4 mL) was stirred at room temperature for 2 h. The reaction was quenched by the addition of water (50 mL) at room temperature. The resulting mixture was extracted with CH₂Cl₂(2×50 mL). The combined organic layers were washed with brine (2×6 mL), dried over anhydrous Na₂SO₄. After filtration, the filtrate was concentrated under reduced pressure. The residue was purified by silica gel column chromatography, eluted with CH₂Cl₂/MeOH (9:1) to afford (S)-1-((8-(3-cyano-3-methylazetidin-1-yl)-3-((1-methyl-1H-pyrazol-4-yl)methyl)-6-(N-(1-methylcyclopropyl)sulfamoyl)-4-oxo-3,4-dihy droquinazolin-2-yl)amino)propan-2-yl methanesulfonate (50 mg, 55% yield, 60% purity) as a yellow solid. LCMS (ESI) m/z: 619 [M+H]⁺.

Step 3: A solution of (S)-1-((8-(3-cyano-3-methylazetidin-1-yl)-3-((1-methyl-1H-pyrazol-4-yl)methyl)-6-(N-(1-methylcyclopropyl)sulfamoyl)-4-oxo-3,4-dihydroquinazolin-2-yl)amino)propan-2-yl methanesulfonate (50 mg, 0.081 mmol) and K₂CO₃ (33.5 mg, 0.24 mmol) in MeCN (5 mL) was stirred at 40° C. for 2h. The reaction was quenched by the addition of water (20 mL) at room temperature. The resulting mixture was extracted with EtOAc (2×20 mL). The combined organic layers were washed with brine (5 mL), dried over anhydrous Na₂SO₄. After filtration, the filtrate was concentrated under reduced pressure. The crude product (50 mg) was purified by Prep-HPLC with the following conditions (Column: XBridge Shield RP18 OBD Column 30*150 mm, 5m; Mobile Phase A: Water (10 mmol/L NH₄HCO₃), Mobile Phase B: ACN; Flow rate: 60 mL/min mL/min; Gradient: 26% B to 42% B in 10 min; Wave Length: 254 nm/220 nm nm; RT1 (min): 14.6) to (R)-9-(3-cyano-3-methylazetidin-1-yl)-1-methyl-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide (9 mg, 21%) as a white solid. LCMS (ESI) m/z: 523.20 [M+H]⁺. ¹H NMR (400 MHz, DMSO-d₆) δ 8.07 (s, 1H), 7.82 (d, J=2.0 Hz, 1H), 7.68 (s, 1H), 7.43 (d, J=0.7 Hz, 1H), 6.88 (d, J=2.0 Hz, 1H), 5.22-5.10 (m, 2H), 4.50 (dd, J=8.3, 3.3 Hz, 2H), 4.15 (d, J=8.3 Hz, 2H), 3.76 (s, 3H), 2.91-2.80 (m, 1H), 2.73 (d, J=6.4 Hz, 1H), 2.38 (d, J=4.2 Hz, 1H), 1.68 (s, 3H), 1.35 (d, J=5.6 Hz, 3H), 1.06 (s, 3H), 0.66-0.57 (m, 2H), 0.40-0.33 (m, 2H).

The following compounds in Table 20 were prepared using procedures similar to those described in Representative Synthesis Route 32 for Example 146 using appropriate starting materials.

TABLE 20

| Example | | |
|---|---|---|
| No. | Name | [M + H]⁺ |
| 126 | (R)-9-((2R,6R)-2,6-dimethylmorpholino)-1,4-dimethyl-N-(1- | 462.10 |
| | methylcyclopropyl)-5-oxo-1,2,4,5-tetrahydroimidazo[1,2- | |
| | a]quinazoline-7-sulfonamide | |

Representative Synthesis Route 33: Example 152: (R)-1-((1-(dimethylamino)cyclopropyl)ethynyl)-4-((1-fluorocyclopropyl)methyl)-N-(1-methylcyclopropyl)-5-oxo-1,2,4,5-tetrahydroimidazo[1,2-a]quinazoline-7-sulfonamide

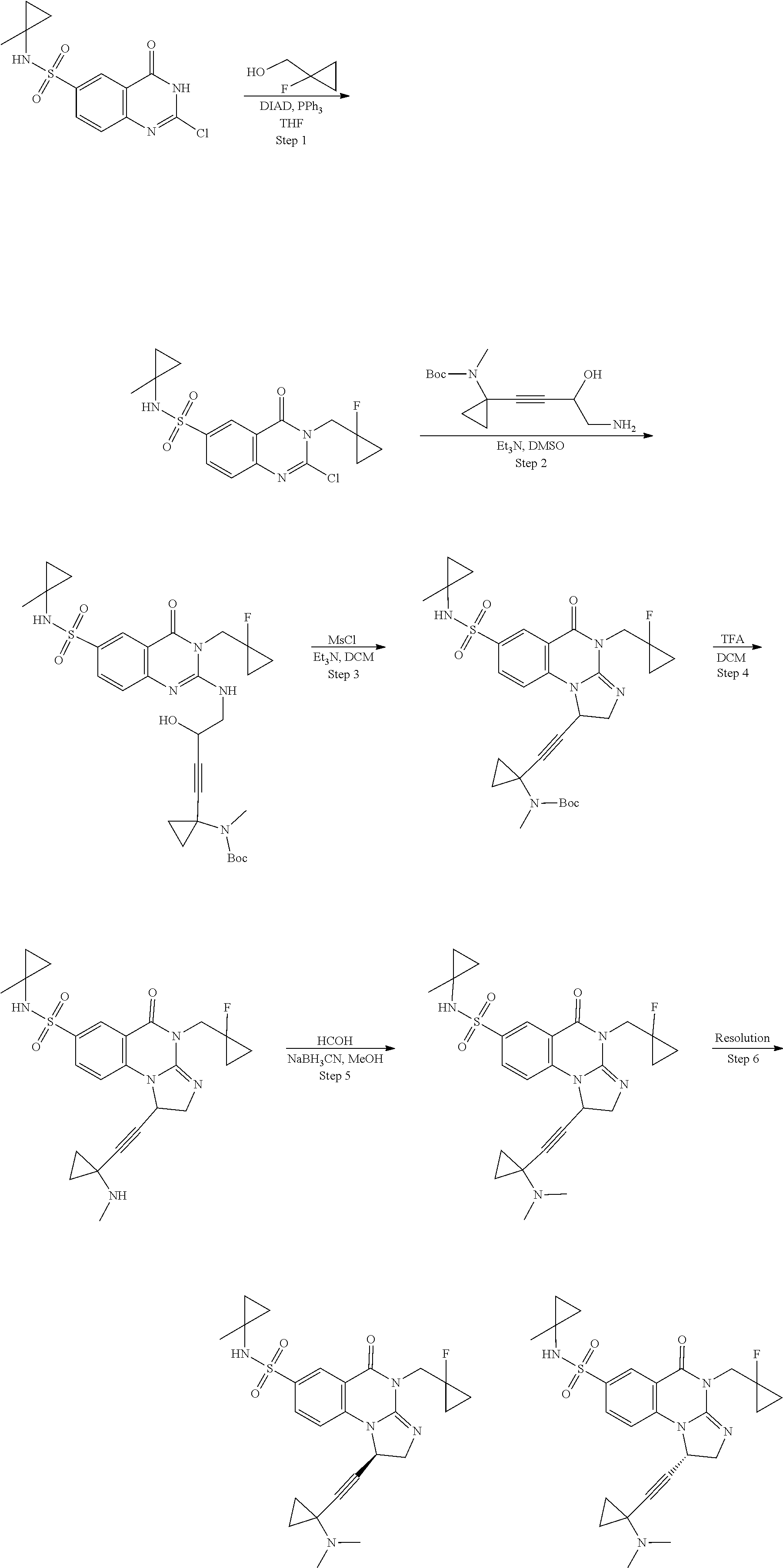

Step 1: To a stirred solution of 2-chloro-N-(1-methylcyclopropyl)-4-oxo-3H-quinazoline-6-sulfonamide (500 mg, 1.59 mmol) in anhydrous THF (20 mL) was added PPh₃ (836 mg, 3.18 mmol) and (1-fluorocyclopropyl)methanol (157.9 mg, 1.75 mmol) followed by the addition of DIAD (644.5 mg, 3.19 mmol) at 0° C. The reaction mixture was stirred at room temperature for 16 h. The reaction was quenched by the addition of water (20 mL) at room temperature. The resulting mixture was extracted with EtOAc (100 mL). The combined organic layers were washed with brine (200 mL), dried over anhydrous MgSO₄. After filtration, the filtrate was concentrated under reduced pressure. The residue was purified by silica gel column chromatography, eluted with PE/EA (1:1) to afford 2-chloro-3-[(1-fluorocyclopropyl)methyl]-N-(1-methylcyclopropyl)-4-oxoquinazoline-6-sulfonamide (250 mg) as a brown solid. LCMS (ESI) m/z: 386 [M+H]⁺.

Steps 2-5 were completed by using procedures similar to those described in Representative Synthesis Route 25 for Example 112.

Step 6: The racemate (70 mg) was purified by Prep-CHIRAL-HPLC with the following conditions (Column: CHIRAL ART Cellulose-SZ, 3*25 cm, 5 m; Mobile Phase A: Hex (10 mM NH₃-MeOH), Mobile Phase B: EtOH; Flow rate: 40 mL/min; Gradient: isocratic 50; Wave Length: 220/254 nm; RT1 (min): 7.72; RT2 (min): 20.56) to afford (R)-1-((1-(dimethylamino)cy clopropyl)ethynyl)-4-((1-fluorocyclopropyl)methyl)-N-(1-methylcyclopropyl)-5-oxo-1,2,4,5-tetrahydroimidazo[1,2-a]quinazoline-7-sulfonamide (28.0 mg) and (S)-1-((1-(dimethylamino)cyclopropyl)ethynyl)-4-((1-fluorocyclopropyl)methyl)-N-(1-methylcyclopropyl)-5-oxo-1,2,4,5-tetrahydroimidazo[1,2-a]quinazoline-7-sulfonamide (28.9 mg) both as white solids. LCMS (ESI) m/z: 500.15 [M+H]⁺. 1H NMR (400 MHz, DMSO-d₆) δ 8.31-8.26 (m, 1H), 8.08-7.98 (m, 2H), 7.38-7.31 (m, 1H), 5.53-5.41 (m, 1H), 4.50-4.33 (m, 2H), 4.26-4.15 (m, 1H), 3.87-3.76 (m, 1H), 2.11 (s, 6H), 1.06-0.94 (m, 7H), 0.84-0.79 (m, 2H), 0.79-0.73 (m, 2H), 0.64-0.56 (m, 2H), 0.41-0.35 (m, 2H).

Representative Synthesis Route 34: Example 163: (R)-4-((1-methyl-1H-pyrazol-4-yl)methyl-d2)-1-((2-methyl-2H-1,2,3-triazol-4-yl)ethynyl)-N-(1-methylcyclopropyl)-5-oxo-1,2,4,5-tetrahydroimidazo[1,2-a]quinazoline-7-sulfonamide

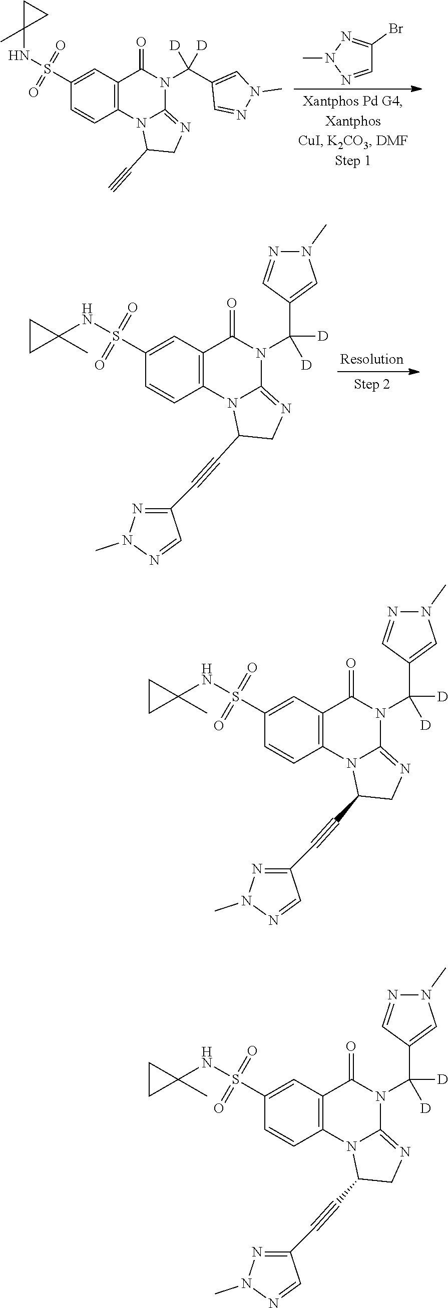

Step 1: To a stirred solution of 1-ethynyl-N-(1-methylcyclopropyl)-4-[(1-methylpyrazol-4-yl)(2H2)methyl]-5-oxo-1H,2H-imidazo[1,2-a]quinazoline-7-sulfonamide (300 mg, 0.68 mmol) and 4-bromo-2-methyl-1,2,3-triazole (132.4 mg, 0.81 mmol) in DMF (15 mL) were added K₂CO₃ (94.5 mg, 0.68 mmol), CuI (22.5 mg, 0.11 mmol) and XantPhos Pd G4 (22 mg, 0.02 mmol), XantPhos (34.1 mg, 0.06 mmol) at room temperature. The resulting mixture was stirred at 90° C. for 2 h under nitrogen atmosphere. The reaction was quenched by the addition of water (50 mL) at room temperature. The resulting mixture was extracted with EtOAc (3×30 mL). The combined organic layers were washed with brine (2×30 mL), dried over anhydrous Na₂SO₄. After filtration, the filtrate was concentrated under reduced pressure. The crude product (200 mg) was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min mL/min; Gradient: 5% B to 5% B in 1.5 min, 5% B to 27% B in 2 min, 27% to 45% B in 10 min; Wave Length: 254 nm/220 nm; RT1 (min): 8.37) to afford 1-[2-(2-methyl-1,2,3-triazol-4-yl)ethynyl]-N-(1-methylcyclopropyl)-4-[(1-methylpyrazol-4-yl)(2H2)methyl]-5-oxo-1H,2H-imidazo[1,2-a]quinazoline-7-sulfonamide (90 mg, 25%) as a yellow solid. LCMS (ESI) m/z: 522.0 [M+H]⁺.

Step 2: The racemate (90 mg) was purified by Chiral-Prep-HPLC with the following conditions (Column: Lux 5u Cellulose-2, 30*250 mm, 5.0 um; Mobile Phase A: Hex (10 mMNNH₃-MeOH), Mobile Phase B: EtOH:CH₃CN=5:1; Flow rate: 40 mL/min; Gradient: isocratic 50; Wave Length: 220/278 nm; RT1 (min): 10.8; RT2 (min): 18.1; Sample Solvent: MeOH) to afford (R)-4-((1-methyl-1H-pyrazol-4-yl)methyl-d2)-1-((2-methyl-2H-1,2,3-triazol-4-yl)ethynyl)-N-(1-methylcyclopropyl)-5-oxo-1,2,4,5-tetrahydroimidazo[1,2-a]quinazoline-7-sulfonamide (30.6 mg, 34%) and (S)-4-((1-methyl-1H-pyrazol-4-yl)methyl-d2)-1-((2-methyl-2H-1,2,3-triazol-4-yl)ethynyl)-N-(1-methylcyclopropyl)-5-oxo-1,2,4,5-tetrahydroimidazo[1,2-a]quinazoline-7-sulfonamide (30.9 mg, 34%) both as white solids. LCMS (ESI) m/z: 522.10 [M+H]⁺. 1H NMR (400 MHz, DMSO-d₆) δ 8.29 (d, J=2.0 Hz, 1H), 8.08 (d, J=5.6 Hz, 1H), 8.03-7.98 (m, 2H), 7.71 (s, 1H), 7.43 (d, J=0.8 Hz, 1H), 7.33 (d, J=8.8 Hz, 1H), 5.70 (dd, J=10.0, 5.2 Hz, 1H), 4.38-4.32 (m, 1H), 4.13 (s, 3H), 4.06-4.03 (m, 1H), 3.77 (s, 3H), 1.06 (s, 3H), 0.60-0.58 (m, 2H), 0.38-0.37 (m, 2H).

Representative Synthesis Route 35: Example 164: (R)-4-((1-methyl-1H-pyrazol-4-yl)methyl)-N-(1-methylcyclopropyl)-1-(3-(methylthio)prop-1-yn-1-yl)-5-oxo-1,2,4,5-tetrahydroimidazo[1,2-a]quinazoline-7-sulfonamide

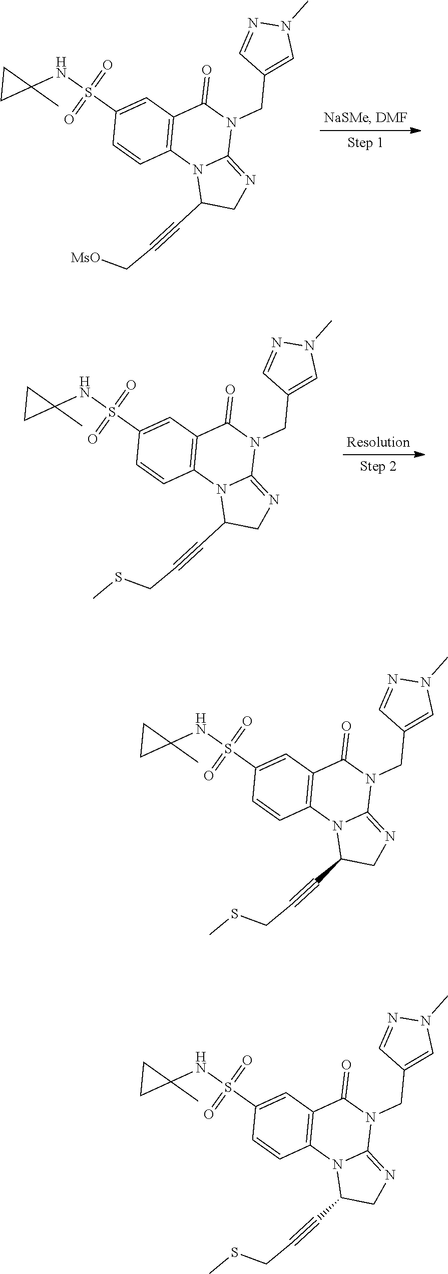 [SMILES: Cn1cc(CN2C(=O)c3cc(S(=O)(=O)NC4(C)CC4)ccc3N3C2=NCC3C#CCOS(C)(=O)=O)cn1 ; CSCC#CC1CN=C2N(Cc3cnn(C)c3)C(=O)c3cc(S(=O)(=O)NC4(C)CC4)ccc3N21 ; CSCC#C[C@@H]1CN=C2N(Cc3cnn(C)c3)C(=O)c3cc(S(=O)(=O)NC4(C)CC4)ccc3N21 ; CSCC#C[C@H]1CN=C2N(Cc3cnn(C)c3)C(=O)c3cc(S(=O)(=O)NC4(C)CC4)ccc3N21]

Step 1: A mixture of 3-(7-[(1-methylcyclopropyl)sulfamoyl]-4-[(1-methylpyrazol-4-yl)methyl]-5-oxo-1H,2H-imidazo[1,2-a]quinazolin-1-ylprop-2-yn-1-yl methanesulfonate (100 mg, 0.18 mmol) and sodium thiomethoxide (25.6 mg, 0.37 mmol) in DMF (2 mL) was stirred at room temperature for 2 h. The reaction was quenched by the addition of water (10 mL) at room temperature and was extracted with EtOAc (2×20 mL). The combined organic layers were washed with brine (2×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in N-(1-methylcyclopropyl)-4-[(1-methylpyrazol-4-yl)methyl]-1-[3-(methylsulfanyl)prop-1-yn-1-yl]-5-oxo-1H,2H-imidazo[1,2-a]quinazoline-7-sulfonamide (15 mg) as a white solid. LCMS (ESI) m/z: 499 [M+H]⁺.

Step 2: The racemate (15 mg) was purified by Prep-CHIRAL-HPLC with the following conditions (Column: CHIRALPAK-IK, 3*25 mm, 5 μm; Mobile Phase A: MTBE (10 mM NH₃-MeOH), Mobile Phase B: EtOH; Flow rate: 40 mL/min; Gradient: isocratic 15; Wave Length: 214/258 nm; RT1 (min): 8.23; RT2 (min): 11.00; Sample Solvent: EtOH: DCM=1:1) to afford (R)-4-((1-methyl-1H-pyrazol-4-yl)methyl)-N-(1-methylcyclopropyl)-1-(3-(methylthio)prop-1-yn-1-yl)-5-oxo-1,2,4,5-tetrahydroimidazo[1,2-a]quinazoline-7-sulfonamide (1.9 mg, 13%) and (S)-4-((1-methyl-1H-pyrazol-4-yl)methyl)-N-(1-methylcyclopropyl)-1-(3-(methylthio)prop-1-yn-1-yl)-5-oxo-1,2,4,5-tetrahydroimidazo[1,2-a]quinazoline-7-sulfonamide (2.4 mg, 16%) both as white solids. LCMS (ESI) m/z: 499.20 [M+H]⁺. ¹H NMR (400 MHz, Methanol-d₄) δ 8.45 (d, J=2.0 Hz, 1H), 8.03 (dd, J=8.8, 2.2 Hz, 1H), 7.68 (s, 1H), 7.55 (s, 1H), 7.35 (d, J=8.8 Hz, 1H), 5.37-5.34 (m, 1H), 5.02 (s, 2H), 4.37-4.26 (m, 1H), 4.10-3.99 (m, 1H), 3.83 (s, 3H), 3.28 (d, J=1.9 Hz, 2H), 2.07 (s, 3H), 1.15 (s, 3H), 0.69-0.63 (m, 2H), 0.44-0.42 (m, 2H).

Representative Synthesis Route 36: Example 177: (R)-1-methyl-N-(1-methylcyclopropyl)-5-oxo-4-((4,5,6,7-tetrahydropyrazolo[1,5-a]pyrazin-3-yl)methyl)-1,2,4,5-tetrahydroimidazo[1,2-a]quinazoline-7-sulfonamide

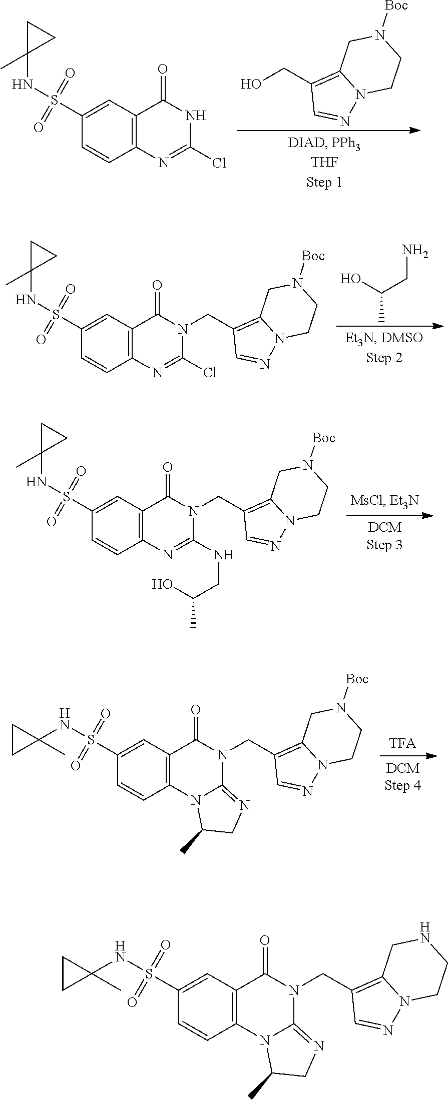

Step 1: To a stirred mixture of 2-chloro-N-(1-methylcyclopropyl)-4-oxo-3H-quinazoline-6-sulfonamide (500 mg, 1.59 mmol) and tert-butyl 3-(hydroxymethyl)-4H,6H,7H-pyrazolo[1,5-a]pyrazine-5-carboxylate (605.5 mg, 2.39 mmol) in THF (15 mL) was added PPh₃ (627 mg, 2.39 mmol) and DIAD (483.4 mg, 2.39 mmol) dropwise at 0° C. The resulting mixture was stirred at room temperature for 16 h. The resulting mixture was concentrated under reduced pressure.

The residue was dissolved in H₂O (100 mL) and extracted with EtOAc (2×100 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (50-80%) to afford tert-butyl 3-({2-chloro-6-[(1-methylcyclopropyl)sulfamoyl]-4-oxoquinazolin-3-yl}methyl)-4H,6H,7H-pyrazolo[1,5-a]pyrazine-5-carboxylate (400 mg, 37%) as a yellow oil. LCMS (ESI) m/z: 549 [M+H]⁺.

Step 2: To a stirred mixture of tert-butyl 3-({2-chloro-6-[(1-methylcyclopropyl)sulfamoyl]-4-oxoquinazolin-3-yl}methyl)-4H,6H,7H-pyrazolo[1,5-a]pyrazine-5-carboxylate (350 mg, 0.64 mmol) and (2S)-1-aminopropan-2-ol (143.6 mg, 1.91 mmol) in DMSO (10 mL) was added Et₃N (193.5 mg, 1.91 mmol) dropwise at room temperature. The resulting mixture was stirred at room temperature for 2 h. The reaction was quenched by the addition of water (50 mL) at room temperature. The resulting mixture was extracted with EtOAc (2×50 mL). The combined organic layers were washed with brine (50 mL), dried over anhydrous Na₂SO₄. After filtration, the filtrate was concentrated under reduced pressure. The residue was purified by silica gel column chromatography, eluted with MeOH/CH₂Cl₂(0-20%) to tert-butyl (S)-3-((2-((2-hydroxypropyl)amino)-6-(N-(1-methylcyclopropyl)sulfamoyl)-4-oxoquinazolin-3(4H)-yl)methyl)-6,7-dihydropyrazolo[1,5-a]pyrazine-5(4H)-carboxylate (256 mg, 68%) as an off-white solid. LCMS (ESI) m/z: 588 [M+H]⁺.

Step 3: To a stirred mixture of tert-butyl (S)-3-((2-((2-hydroxypropyl)amino)-6-(N-(1-methylcyclopropyl)sulfamoyl)-4-oxoquinazolin-3(4H)-yl)methyl)-6,7-dihydropyrazolo[1,5-a]pyrazine-5(4H)-carboxylate (256 mg, 0.44 mmol) and Et₃N (176.3 mg, 1.74 mmol) in DCM (8 mL) was added methanesulfonyl chloride (274.4 mg, 2.40 mmol) dropwise at room temperature. The resulting mixture was stirred at room temperature for 4 h. The reaction was quenched by the addition of water (50 mL) at room temperature. The resulting mixture was extracted with EtOAc (2×50 mL). The combined organic layers were washed with brine (50 mL), dried over anhydrous Na₂SO₄. After filtration, the filtrate was concentrated under reduced pressure. This resulted in tert-butyl (R)-3-((1-methyl-7-(N-(1-methylcyclopropyl)sulfamoyl)-5-oxo-1,2-dihy droimidazo[1,2-a]quinazolin-4(5H)-yl)methyl)-6,7-dihydropyrazolo[1,5-a]pyrazine-5(4H)-carboxylate (307 mg) as a yellow solid was used in the next step directly without further purification. LCMS (ESI) m/z: 570 [M+H]⁺.

Step 4: A mixture of tert-butyl (R)-3-((1-methyl-7-(N-(1-methylcyclopropyl)sulfamoyl)-5-oxo-1,2-dihydroimidazo[1,2-a]quinazolin-4(5H)-yl)methyl)-6,7-dihydropyrazolo[1,5-a]pyrazine-5(4H)-carboxylate (307 mg, 0.54 mmol) in DCM (6 mL) and TFA (2 mL) was stirred at room temperature for 1 h. The resulting mixture was concentrated under reduced pressure. The crude product (250 mg) was purified by Prep-HPLC with the following conditions (Column: Lux 5u Cellulose-2, 30*250 mm, 5.0 um; Mobile Phase A: Hex (10 mMNH₃-MeOH), Mobile Phase B: EtOH: CH₃CN=5:1; Flow rate: 40 mL/min; Gradient: isocratic 50; Wave Length: 232/276 nm; RT1 (min): 8.588; RT2 (min): 11.259; Sample Solvent: EtOH: MeOH=2:1) to afford (R)-1-methyl-N-(1-methylcyclopropyl)-5-oxo-4-((4,5,6,7-tetrahydropyrazolo[1,5-a]pyrazin-3-yl)methyl)-1,2,4,5-tetrahydroimidazo[1,2-a]quinazoline-7-sulfonamide (42 mg, 17%) as a white solid. LCMS (ESI) m/z: 470.05 [M+H]⁺. ¹H NMR (400 MHz, DMSO-d₆) δ 8.24 (d, J=2.2 Hz, 1H), 8.02 (s, 1H), 7.90 (d, J=8.0 Hz, 1H), 7.42 (s, 1H), 7.23 (d, J=8.0 Hz, 1H), 4.90-4.80 (m, 2H), 4.73-4.69 (m, 1H), 4.07-4.01 (m, 3H), 3.90 (t, J=5.5 Hz, 2H), 3.54-3.51 (m, 1H), 3.05 (t, J=5.3 Hz, 2H), 1.31 (d, J=4.0 Hz, 3H), 1.06 (s, 3H), 0.60-0.58 (m, 2H), 0.38-0.36 (m, 2H).

The following compounds in Table 21 were prepared using procedures similar to those described in Representative Synthesis Route 36 for Example 177 using appropriate starting

TABLE 21

| Example | | |
|---|---|---|
| No. | Name | [M + H]⁺ |
| 162 | (R)-1-methyl-N-(1-methylcyclopropyl)-5-oxo-4-((1-(prop-2- | 453.20 |
| | yn-1-yl)-1H-pyrazol-4-yl)methyl)-1,2,4,5- | |
| | tetrahydroimidazo[1,2-a]quinazoline-7-sulfonamide | |
| 230 | (R)-4-((3-cyclopropyl-1-methyl-1H-pyrazol-4-yl)methyl)-1- | 469.20 |
| | methyl-N-(1-methylcyclopropyl)-5-oxo-1,2,4,5- | |
| | tetrahydroimidazo[1,2-a]quinazoline-7-sulfonamide | |
| 288 | (R)-1-((2-methyl-2H-1,2,3-triazol-4-yl)ethynyl)-N-(1- | 561.15 |
| | methylcyclopropyl)-5-oxo-4-((4,5,6,7-tetrahydropyrazolo[1,5- | |
| | a]pyrazin-3-yl)methyl)-1,2,4,5-tetrahydroimidazo[1,2- | |
| | a]quinazoline-7-sulfonamide | |
| 290 | (7aS,10aR)-N-(1-methylcyclopropyl)-5-oxo-6-((4,5,6,7- | 496.00 |
| | tetrahydropyrazolo[1,5-a]pyrazin-3-yl)methyl)- | |
| | 6,7a,8,9,10,10a-hexahydro-5H-cyclopenta[4,5]imidazo[1,2- | |
| | a]quinazoline-3-sulfonamide | |
| 297 | (R)-9-chloro-1-ethynyl-N-(1-methylcyclopropyl)-5-oxo-4- | 514.05 |
| | ((4,5,6,7-tetrahydropyrazolo[1,5-a]pyrazin-3-yl)methyl)- | |
| | 1,2,4,5-tetrahydroimidazo[1,2-a]quinazoline-7-sulfonamide | |

Representative Synthesis Route 37: Example 187: (R)-4-ethyl-I-((2-methyl-2H-1,2,3-triazol-4-yl)ethynyl)-N-(1-methylcyclopropyl)-5-oxo-1,2,4,5-tetrahydroimidazo[1,2-a]quinazoline-7-sulfonamide

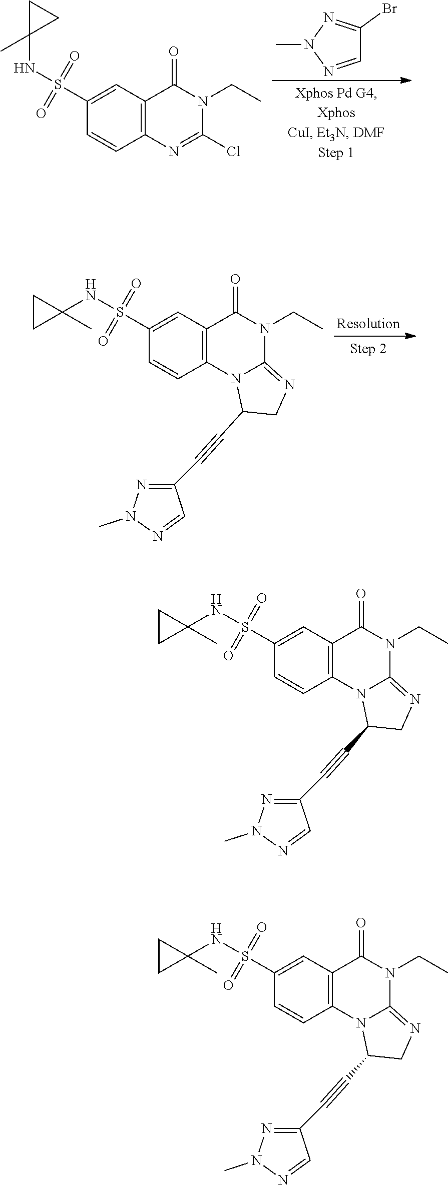

Step 1: A solution of 4-ethyl-1-ethynyl-N-(1-methylcyclopropyl)-5-oxo-1H,2H-imidazo[1,2-a]quinazoline-7-sulfonamide (100 mg, 0.29 mmol), 4-bromo-2-methyl-1,2,3-triazole (52.2 mg, 0.32 mmol), XPhos Pd G4 (23.1 mg, 0.027 mmol), XPhos (12.8 mg, 0.027 mmol), Et₃N (54.3 mg, 0.54 mmol) in DMF (10 mL) was treated with Cul (2.6 mg, 0.013 mmol) at room temperature for 2 min. The resulting mixture was stirred at 80° C. overnight under nitrogen atmosphere. The reaction was quenched by the addition of water (30 mL) at room temperature. The resulting mixture was extracted with CH₂Cl₂ (3×100 mL). The combined organic layers were washed with brine (2×100 mL), dried over anhydrous MgSO₄. After filtration, the filtrate was concentrated under reduced pressure. The residue was purified by reversed-phase flash chromatography with the following conditions: column, C18 silica gel; mobile phase, MeCN in Water (0.1% FA), 20% to 60% gradient in 30 min; detector, UV 254 nm. This resulted in 4-ethyl-1-((2-methyl-2H-1,2,3-triazol-4-yl)ethynyl)-N-(1-methylcyclopropyl)-5-oxo-1,2,4,5-tetrahydroimidazo[1,2-a]quinazoline-7-sulfonamide (40 mg, 30%) as a white solid. LCMS (ESI) m/z: 454 [M+H]⁺.

Step 2: The racemate (40 mg) was purified by Prep-CHIRAL-HPLC with the following conditions (Column: XA-Lux 5 μm Cellulose-2, 3*25 cm, 5 m; Mobile Phase A: Hex (10 mM NH₃-MeOH), Mobile Phase B: EtOH: CH₃CN=5:1; Flow rate: 40 mL/min; Gradient: isocratic 50; Wave Length: 224/260 nm; RT1 (min): 6.699; RT2 (min): 10.154; Sample Solvent: MeOH: EtOH=3:2) to afford (R)-4-ethyl-1-((2-methyl-2H-1,2,3-triazol-4-yl)ethynyl)-N-(1-methylcyclopropyl)-5-oxo-1,2,4,5-tetrahydroimidazo[1,2-a]quinazoline-7-sulfonamide (10.4 mg, 26%) and (S)-4-ethyl-1-((2-methyl-2H-1,2,3-triazol-4-yl)ethynyl)-N-(1-methylcyclopropyl)-5-oxo-1,2,4,5-tetrahydroimidazo[1,2-a]quinazoline-7-sulfonamide (10.8 mg, 27%) both as white solids. LCMS (ESI) m/z: 454.20 [M+H]⁺. 1H NMR (400 MHz, DMSO-d₆) δ 8.27 (d, J=2.4 Hz, 1H), 8.09 (s, 1H), 8.03-7.99 (m, 2H), 7.35-7.33 (m, 1H), 5.71-5.67 (m, 1H), 4.35-4.29 (m, 1H), 4.13 (s, 3H), 4.04-3.95 (m, 3H), 1.23-1.20 (m, 3H), 1.08 (s, 3H), 0.61-0.59 (m, 2H), 0.40-0.37 (m, 2H).

The following compounds in Table 22 were prepared using procedures similar to those described in Representative Synthesis Route 37 for Example 187 using appropriate starting materials.

TABLE 22

| Example | | |
|---|---|---|
| No. | Name | [M + H]⁺ |
| 195 | (R)-1-((3-((dimethylamino)methyl)-1-methyl-1H-pyrazol-5- | 510.30 |
| | yl)ethynyl)-4-ethyl-N-(1-methylcyclopropyl)-5-oxo-1,2,4,5- | |
| | tetrahydroimidazo[1,2-a]quinazoline-7-sulfonamide | |
| 198 | (R)-4-ethyl-1-((1-methyl-1H-pyrazol-5-yl)ethynyl)-N-(1- | 453.15 |
| | methylcyclopropyl)-5-oxo-1,2,4,5-tetrahydroimidazo[1,2- | |
| | a]quinazoline-7-sulfonamide | |
| 207 | (R)-1-((1-ethyl-1H-pyrazol-5-yl)ethynyl)-4-((1-methyl-1H- | 533.20 |
| | pyrazol-4-yl)methyl)-N-(1-methylcyclopropyl)-5-oxo-1,2,4,5- | |
| | tetrahydroimidazo[1,2-a]quinazoline-7-sulfonamide | |
| 208 | (R)-4-ethyl-1-((2-methyl-2H-pyrazolo[4,3-b]pyridin-3- | 504.10 |
| | yl)ethynyl)-N-(1-methylcyclopropyl)-5-oxo-1,2,4,5- | |
| | tetrahydroimidazo[1,2-a]quinazoline-7-sulfonamide | |
| 211 | (R)-4-((1-methyl-1H-pyrazol-4-yl)methyl)-1-((1-(methyl-d3)- | 522.25 |
| | 1H-pyrazol-5-yl)ethynyl)-N-(1-methylcyclopropyl)-5-oxo- | |
| | 1,2,4,5-tetrahydroimidazo[1,2-a]quinazoline-7-sulfonamide | |
| 218 | (R)-1-((1,3-dimethyl-1H-pyrazol-4-yl)ethynyl)-4-((1-methyl- | 533.25 |
| | 1H-pyrazol-4-yl)methyl)-N-(1-methylcyclopropyl)-5-oxo- | |
| | 1,2,4,5-tetrahydroimidazo[1,2-a]quinazoline-7-sulfonamide | |
| 228 | (R)-1-((3-cyclopropyl-1-methyl-1H-pyrazol-5-yl)ethynyl)-4- | 493.20 |
| | ethyl-N-(1-methylcyclopropyl)-5-oxo-1,2,4,5- | |
| | tetrahydroimidazo[1,2-a]quinazoline-7-sulfonamide | |
| 232 | (R)-1-((1,3-dimethyl-1H-pyrazol-4-yl)ethynyl)-4-((1-methyl- | 535.20 |
| | 1H-pyrazol-4-yl)methyl-d2)-N-(1-methylcyclopropyl)-5-oxo- | |
| | 1,2,4,5-tetrahydroimidazo[1,2-a]quinazoline-7-sulfonamide | |
| 257 | (R)-1-((2,5-dimethyl-2H-1,2,3-triazol-4-yl)ethynyl)-4-ethyl-N- | 468.20 |
| | (1-methylcyclopropyl)-5-oxo-1,2,4,5-tetrahydroimidazo[1,2- | |
| | a]quinazoline-7-sulfonamide | |
| 265 | (R)-1-((1,3-dimethyl-1H-pyrazol-4-yl)ethynyl)-4-ethyl-N-(1- | 467.20 |
| | methylcyclopropyl)-5-oxo-1,2,4,5-tetrahydroimidazo[1,2- | |
| | a]quinazoline-7-sulfonamide | |
| 266 | (R)-1-((2,5-dimethyl-2H-1,2,3-triazol-4-yl)ethynyl)-4-((1- | 536.25 |
| | methyl-1H-pyrazol-4-yl)methyl-d2)-N-(1-methylcyclopropyl)- | |
| | 5-oxo-1,2,4,5-tetrahydroimidazo[1,2-a]quinazoline-7- | |
| | sulfonamide | |

Representative Synthesis Route 38: Example 205: (1R)-4-ethyl-1-((6-(methyl amino)-6,7-dihydro-5H-pyrazolo[5,i-b][1,3]oxazin-3-yl)ethynyl)-N-(1-methylcyclopropyl)-5-oxo-1,2,4,5-tetrahydroimidazo[1,2-a]quinazoline-7-sulfonamide

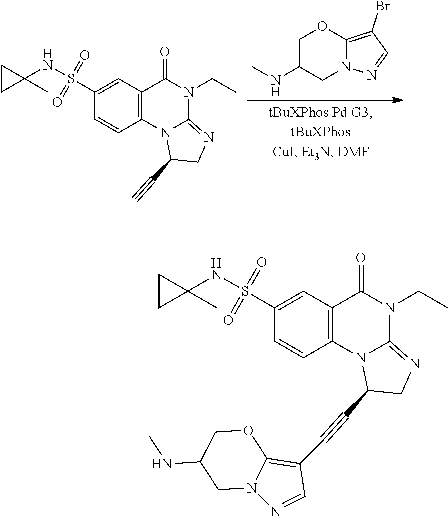

A mixture of (1R)-4-ethyl-1-ethynyl-N-(1-methylcyclopropyl)-5-oxo-1H,2H-imidazo[1,2-a]quinazoline-7-sulfonamide (120 mg, 0.32 mmol), 3-brom o-N-methyl-5H, 6H, 7H-pyrazol o[3,2-b][1,3]oxazin-6-amine(74.8 mg, 0.32 mmol), Et₃N (65.2 mg, 0.64 mmol), CuI (3.1 mg, 0.016 mmol), t-BuXPhos Pd G3 (25.6 mg, 0.032 mmol) and t-BuXPhos (13.7 mg, 0.032 mmol) in DMF (1.5 mL) was stirred at 80° C. for 2 h under nitrogen atmosphere. The reaction was quenched by the addition of water (50 mL) at room temperature. The resulting mixture was extracted with EtOAc (2×50 mL). The combined organic layers were washed with brine (2×10 mL), dried over anhydrous Na₂SO₄. After filtration, the filtrate was concentrated under reduced pressure. The crude product (120 mg) was purified by Prep-HPLC with the following conditions (Column: XBridge Prep OBD C18 Column 30*150 mm, 5 m; Mobile Phase A: Water (10 mmol/L NH₄HCO₃), Mobile Phase B: CH₃CN; Flow rate: 60 mL/min mL/min; Gradient: 21% B to 37% B in 16 min; Wave Length: 254 nm/220 nm; RT 1(min): 11.68) to afford (1R)-4-ethyl-1-((6-(methylamino)-6,7-dihydro-5H-pyrazolo[5,1-b][1,3]oxazin-3-yl)ethynyl)-N-(1-methylcyclopropyl)-5-oxo-1,2,4,5-tetrahydroimidazo[1,2-a]quinazoline-7-sulfonamide (7.3 mg, 4%) as a white solid. LCMS (ESI) m/z: 524.10 [M+H]⁺. ¹H NMR (400 MHz, DMSO-d₆) δ 8.27 (d, J=2.2 Hz, 1H), 8.06 (s, 1H), 8.00 (dd, J=8.6, 2.2 Hz, 1H), 7.40 (s, 1H), 7.31 (d, J=8.6 Hz, 1H), 5.59-5.55 (m, 1H), 4.30-4.19 (m, 3H), 4.14 (dd, J=12.4, 4.4 Hz, 1H), 4.02-3.94 (m, 2H), 3.92-3.84 (m, 2H), 3.17-3.05 (m, 1H), 2.30 (s, 3H), 2.07 (s, 1H), 1.21 (t, J=7.0 Hz, 3H), 1.08 (s, 3H), 0.60-0.59 (m, 2H), 0.41-0.36 (m, 2H).

Representative Synthesis Route 39: Example 213: (7aS,10aR)-2-methoxy-6-((1-methyl-1H-pyrazol-4-yl)methyl-d2)-N-(1-methylcyclopropyl)-5-oxo-6,7a,8,9,10,1Oa-hexahydro-5H-cyclopenta[4,5]imidazo[1,2-a]quinazoline-3-sulfonamide

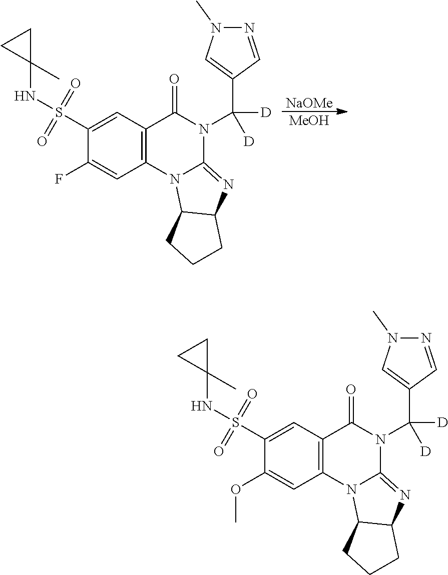 [SMILES: "MeOH" ; [2H]C([2H])(c1cnn(C)c1)N1C(=O)c2cc(S(=O)(=O)NC3(C)CC3)c(F)cc2N2C1=N[C@H]1CCC[C@H]12 ; [2H]C([2H])(c1cnn(C)c1)N1C(=O)c2cc(S(=O)(=O)NC3(C)CC3)c(OC)cc2N2C1=N[C@H]1CCC[C@H]12 ; "NaOMe"]

To a solution of (7aS,10aR)-2-fluoro-6-((1-methyl-1H-pyrazol-4-yl)methyl-d2)-N-(1-methylcyclopropyl)-5-oxo-6,7a,8,9,10,10a-hexahydro-5H-cyclopenta[4,5]imidazo[1,2-a]quinazoline-3-sulfonamide (7 mg, 0.015 mmol) in MeOH (0.14 mL) added NaOMe (1.6 mg, 0.029 mmol). The reaction was stirred at room temperature for 1 hour. The solution was quenched with a saturated aqueous solution of ammonium chloride. The mixture was extracted with EtOAc. The organic phases were combined, dried over sodium sulfate, and the solvent was removed in vacuo. The resultant residue was purified via silica gel column chromatography {(0-100%, Hex: [25% EtOH in EtOAc])} to afford (7aS,10aR)-2-methoxy-6-((1-methyl-1H-pyrazol-4-yl)methyl-d2)-N-(1-methylcyclopropyl)-5-oxo-6,7a,8,9,10,10a-hexahydro-5H-cyclopenta[4,5]imidazo[1,2-a]quinazoline-3-sulfonamide as a white solid (5.6 mg, 78%). LCMS (ESI) m/z: 487.00 [M+H]⁺. ¹H NMR (400 MHz, DMSO-d₆) δ 8.19 (s, 1H), 7.72 (s, 1H), 7.67 (s, 1H), 7.40 (s, 1H), 6.49 (s, 1H), 4.85-4.83 (m, 1H), 4.66-4.63 (m, 1H), 3.98 (s, 3H), 3.76 (s, 3H), 2.06-1.83 (m, 4H), 1.41-1.39 (m, 2H), 1.07 (s, 3H), 0.64-0.58 (m, 2H), 0.40-0.34 (m, 2H).

Representative Synthesis Route 40: Example 214: (7aS,10aR)-2-(dimethylamino)-6-((1-methyl-1H-pyrazol-4-yl)methyl-d2)-N-(1-methylcyclopropyl)-5-oxo-6,7a,8,9,10,10a-hexahydro-5H-cyclopenta[4,5]imidazo[1,2-a]quinazoline-3-sulfonamide

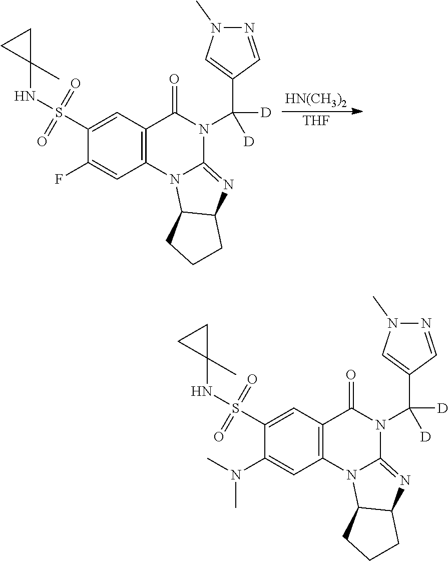

To (7aS,10aR)-2-fluoro-6-((1-methyl-1H-pyrazol-4-yl)methyl-d2)-N-(1-methylcyclopropyl)-5-oxo-6,7a,8,9,10,10a-hexahydro-5H-cyclopenta[4,5]imidazo[1,2-a]quinazoline-3-sulfonamide (5 mg, 0.01 mmol) added 2M dimethyl amine in THF (15.8 uL, 0.032 mmol). The reaction was heated to 50° C. and stirred for 30 minutes. The solution was quenched with a saturated aqueous solution of ammonium chloride. The mixture was extracted with EtOAc. The organic phases were combined, dried over sodium sulfate, and the solvent was removed in vacuo. The resultant residue was purified by silica gel column chromatography (0-100%, Hex: [25% EtOH in EtOAc]) to afford (7aS,10aR)-2-(dimethylamino)-6-((1-methyl-1H-pyrazol-4-yl)methyl-d2)-N-(1-methylcyclopropyl)-5-oxo-6,7a,8,9,10,10a-hexahydro-5H-cyclopenta[4,5]imidazo[1,2-a]quinazoline-3-sulfonamide (3.6 mg, 68%). LCMS (ESI) m/z: 500.00 [M+H]⁺. 1H NMR (400 MHz, DMSO-d₆) δ 8.35 (s, 1H), 7.90-7.70 (m, 2H) 7.37-7.33 (m, 1H), 6.60 (s, 1H), 4.72-4.60 (m, 1H), 3.77 (s, 3H), 2.88 (bs, 6H), 1.88-1.85 (m, 4H), 1.65-1.50 (m, 1H), 1.30-1.24 (m, 1H), 1.05 (s, 3H), 0.58-0.56 (m, 2H) 0.37-0.35 (m, 2H).

The following compounds in Table 23 were prepared using procedures similar to those described in Representative Synthesis Route 40 for Example 214 using appropriate starting

TABLE 23

| Example | | |
|---|---|---|
| No. | Name | [M + H]⁺ |
| 222 | (7aS,10aR)-6-((1-methyl-1H-pyrazol-4-yl)methyl-d2)-2- | 486.00 |
| | (methylamino)-N-(1-methylcyclopropyl)-5-oxo- | |
| | 6,7a,8,9,10,10a-hexahydro-5H-cyclopenta[4,5]imidazo[1,2- | |
| | a]quinazoline-3-sulfonamide | |
| 234 | (7aS,10aR)-2-amino-6-((1-methyl-1H-pyrazol-4-yl)methyl- | 472.00 |
| | d2)-N-(1-methylcyclopropyl)-5-oxo-6,7a,8,9,10,10a- | |
| | hexahydro-5H-cyclopenta[4,5]imidazo[1,2-a]quinazoline-3- | |
| | sulfonamide | |
| 286 | (7aS,10aR)-2-(((6-methoxypyridin-3-yl)methyl)amino)-6-((1- | 593.00 |
| | methyl-1H-pyrazol-4-yl)methyl-d2)-N-(1-methylcyclopropyl)- | |
| | 5-oxo-6,7a,8,9,10,10a-hexahydro-5H- | |
| | cyclopenta[4,5]imidazo[1,2-a]quinazoline-3-sulfonamide | |

Representative Synthesis Route 41: Example 237: (R)-N-(1-(hydroxymethyl)cyclopropyl)-1-methyl-4-((1-methyl-1H-pyrazol-4-yl)methyl-d2)-5-oxo-1,2,4,5-tetrahydroimidazo[1,2-a]quinazoline-7-sulfonamide

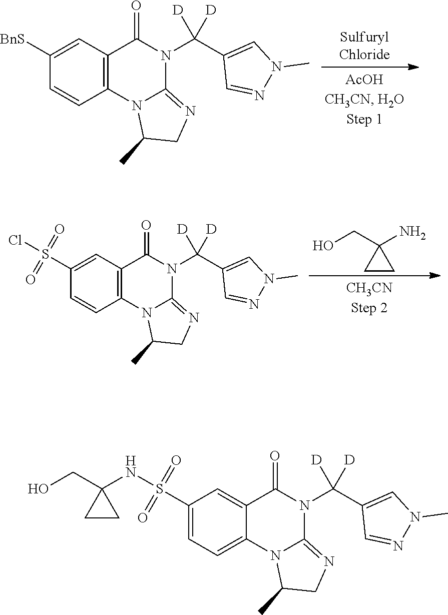

Step 1: A solution of (1R)-7-(benzylsulfanyl)-1-methyl-4-[(1-methylpyrazol-4-yl)(2H2)methyl]-1H,2H-imidazo[1,2-a]quinazolin-5-one (1 g, 2.38 mmol) and sulfuroyl dichloride (1.61 g, 11.9 mmol) in H₂O (0.5 mL), MeCN (20 mL) and HOAc (0.75 mL) was stirred at 0° C. for 2 h. The resulting mixture was concentrated under reduced pressure. This resulted in (1R)-1-methyl-4-[(1-methylpyrazol-4-yl)(2H2)methyl]-5-oxo-1H,2H-imidazo[1,2-a]quinazoline-7-sulfonyl chloride (1 g) as a yellow liquid. The resulting mixture was used in the next step directly without further purification. LCMS (ESI) m/z: 396 [M+H]⁺.

Step 2: A solution of (1R)-1-methyl-4-[(1-methylpyrazol-4-yl)(2H2)methyl]-5-oxo-1H,2H-imidazo[1,2-a]quinazoline-7-sulfonyl chloride (1 g, 2.53 mmol) and (1-aminocyclopropyl)methanol (4.4 g, 50.5 mmol) in MeCN (15 mL) was stirred at 0° C. for 1 h. The reaction was quenched by the addition of water (50 mL) at room temperature. The resulting mixture was extracted with EtOAc (2×50 mL). The combined organic layers were washed with brine (2×10 mL), dried over anhydrous Na₂SO₄. After filtration, the filtrate was concentrated under reduced pressure. The crude product (800 mg) was purified by Prep-HPLC with the following conditions (Column: XBridge Prep OBD C18 Column 30*150 mm, 5 m; Mobile Phase A: Water (10 mmol/L NH₄HCO₃), Mobile Phase B: ACN; Flow rate: 60 mL/min mL/min; Gradient: 10% B to 29% B in 16 min; Wave Length: 254 nm/220 nm; RTl (min): 11.43) to afford (1R)-N-[1-(hydroxymethyl)cyclopropyl]-1-methyl-4-[(1-methylpyrazol-4-yl)(2H2)methyl]-5-oxo-1H,2H-imidazo[1,2-a]quinazoline-7-sulfonamide (94.6 mg, 8%) as a white solid. LCMS (ESI) m/z: 447.05 [M+H]⁺. ¹H NMR (400 MHz, DMSO-d₆) δ 8.22 (d, J=2.2 Hz, 1H), 8.13 (s, 1H), 7.90 (dd, J=8.8, 2.2 Hz, 1H), 7.68 (d, J=0.8 Hz, 1H), 7.41 (d, J=0.8 Hz, 1H), 7.22 (d, J=8.8 Hz, 1H), 4.76-4.66 (m, 1H), 4.57 (t, J=5.8 Hz, 1H), 4.08-4.02 (m, 1H), 3.76 (s, 3H), 3.54-3.49 (m, 1H), 3.27 (d, J=5.6 Hz, 2H), 1.32 (d, J=6.2 Hz, 3H), 0.60-0.53 (m, 2H), 0.52-0.46 (m, 2H).

Representative Synthesis Route 42: Example 240: (7aS,10aR)-6-((1-methyl-1H-pyrazol-4-yl)methyl-d2)-N-(1-methylcyclopropyl)-2-(methylthio)-5-oxo-6,7a,8,9,10,1Oa-hexahydro-5H-cyclopenta[4,5]imidazo[1,2-a]quinazoline-3-sulfonamide

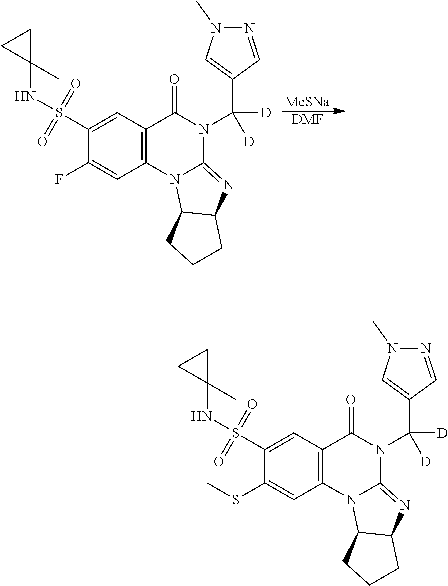

To a solution of (7aS,10aR)-2-fluoro-6-((1-methyl-1H-pyrazol-4-yl)methyl-d2)-N-(1-methylcyclopropyl)-5-oxo-6,7a,8,9,10,10a-hexahydro-5H-cyclopenta[4,5]imidazo[1,2-a]quinazoline-3-sulfonamide (20 mg, 0.042 mmol) in DMF (0.3 mL) was added sodium methanethiolate (8.9 mg, 0.13 mmol). The reaction was stirred at room temperature for 1 hour. The mixture was quenched with a saturated aqueous solution of ammonium chloride and was extracted with EtOAc. The organic phases were combined and washed with water and brine. The organic phase was dried over sodium sulfate and had the solvent removed in vacuo. The resultant residue was purified by silica gel column chromatography (0-100% Hex [25% EtOH in EtOAc]) to afford (7aS,10aR)-6-((1-methyl-1H-pyrazol-4-yl)methyl-d2)-N-(1-methylcyclopropyl)-2-(methylthio)-5-oxo-6,7a,8,9,10,10a-hexahydro-5H-cyclopenta[4,5]imidazo[1,2-a]quinazoline-3-sulfonamide, as a white solid (13 mg, 61%). LCMS (ESI) m/z: 503.00 [M+H]⁺. ¹H NMR (400 MHz, DMSO-d₆) δ 8.28 (s, 1H), 8.03 (s, 1H) 7.67 (s, 1H), 7.41 (s, 1H), 6.67 (s, 1H), 4.90-4.80 (m, 1H), 4.66-4.64 (m, 1H), 3.76 (s, 1H), 2.59 (s, 3H), 1.92-1.90 (m, 4H), 1.65-1.58 (m, 2H), 1.51-1.43 (m, 2H), 1.08 (s, 3H), 0.59-0.58 (m, 2H), 0.37-0.35 (m, 2H).

Representative Synthesis Route 43: Example 243: (R)-4-((3-(cyanomethyl)-1-methyl-1H-pyrazol-4-yl)methyl)-1-methyl-N-(1-methylcyclopropyl)-5-oxo-1,2,4,5-tetrahydroimidazo[1,2-a]quinazoline-7-sulfonamide

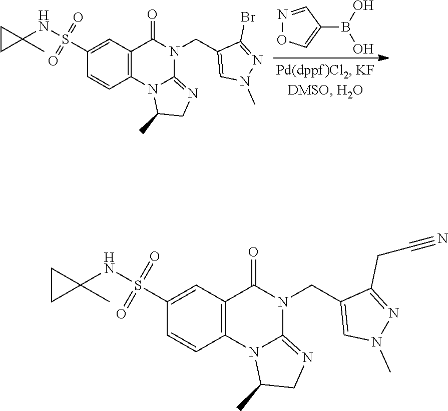

A mixture of (1R)-4-[(3-bromo-1-methylpyrazol-4-yl)methyl]-1-methyl-N-(1-methylcyclopropyl)-5-oxo-1H,2H-imidazo[1,2-a]quinazoline-7-sulfonamide (100 mg, 0.2 mmol), 4-(4,4,5,5-tetramethyl-1,3,2-dioxaborolan-2-yl)-1,2-oxazole (57.6 mg, 0.29 mmol), KF (34.3 mg, 0.59 mmol), Pd(dppf)Cl₂ (14.4 mg, 0.02 mmol) in DMSO (1 mL) and H₂O (0.2 mL) was stirred for 5 h at 130° C. under nitrogen atmosphere. The resulting mixture was filtered, the filter cake was washed with MeOH (3×10 mL). The filtrate was concentrated under reduced pressure. The residue was purified by reversed-phase flash chromatography with the following conditions: Column: Xselect CSH Prep C18, 30*150 mm 5 m; Mobile Phase A: Water (0.1% FA), Mobile Phase B: CH₃CN; Flow rate: 60 mL/min mL/min; Gradient: isocratic 2% to 20% B in 14 min; Wave Length: 254 nm/220 nm; RT1 (min): 13.65 min. This resulted in (R)-4-((3-(cyanomethyl)-1-methyl-1H-pyrazol-4-yl)methyl)-1-methyl-N-(1-methylcyclopropyl)-5-oxo-1,2,4,5-tetrahydroimidazo[1,2-a]quinazoline-7-sulfonamide (7.0 mg, 8%) as a white solid. LCMS (ESI) m/z: 468.15 [M+H]⁺. 1H NMR (400 MHz, Methanol-d₄) δ 8.42 (d, J=2.2 Hz, 1H), 7.99 (dd, J=8.7, 2.2 Hz, 1H), 7.72 (s, 1H), 7.19 (d, J=8.7 Hz, 1H), 5.01 (s, 2H), 4.73 (td, J=6.3, 3.2 Hz, 1H), 4.17 (dd, J=13.5, 9.3 Hz, 1H), 4.10 (d, J=1.6 Hz, 2H), 3.81 (s, 3H), 3.64 (dd, J=13.5, 3.5 Hz, 1H), 1.41 (d, J=6.3 Hz, 3H), 1.15 (s, 3H), 0.74-0.67 (m, 2H), 0.46-0.38 (m, 2H).

Representative Synthesis Route 44: Example 244: (R)-1-((1-((2-hydroxyethyl)(methyl)amino)cyclopropyl)ethynyl)-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide

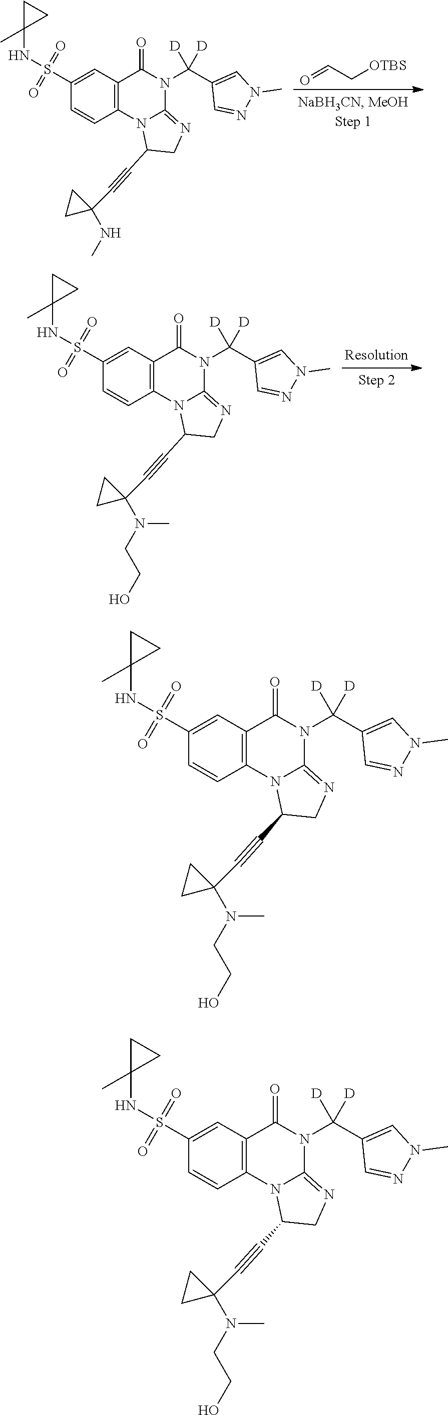

Step 1: A mixture of 4-((1-methyl-1H-pyrazol-4-yl)methyl-d2)-1-((1-(methylamino)cyclopropyl)ethynyl)-N-(1-methylcyclopropyl)-5-oxo-1,2,4,5-tetrahydroimidazo[1,2-a]quinazoline-7-sulfonamide (500 mg, 0.98 mmol) and 2-[(tert-butyldimethylsilyl)oxy]acetaldehyde (342 mg, 1.96 mmol) in MeOH (7 mL) was stirred at room temperature for 1 h. To the above mixture was added NaBH₃CN (185 mg, 2.94 mmol) at room temperature. The resulting mixture was stirred at room temperature for additional 4 h. The mixture was basified to pH 8 with saturated NaHCO₃ (aq.). The resulting mixture was extracted with EtOAc (2×50 mL). The combined organic layers were washed with brine (10 mL), dried over anhydrous Na₂SO₄. After filtration, the filtrate was concentrated under reduced pressure. The crude product (100 mg) was purified by Prep-HPLC with the following conditions (Column: XselectCSH Prep C18, 30*150 mm 5 m; Mobile Phase A: Water (0.1% FA), Mobile Phase B: CH₃CN; Flow rate: 60 mL/min mL/min; Gradient: isocratic 2% to 17% B in 10 min; Wave Length: 254 nm/220 nm; RT1 (min): 10.70 min) to afford 1-((1-((2-hydroxyethyl)(methyl)amino)cyclopropyl)ethynyl)-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide (20 mg, 4%) as a white solid. LCMS (ESI) m/z: 554 [M+H]⁺.

Step 2: The racemate (20 mg) was purified by Prep-Chiral-HPLC with the following conditions Column: CHIRALPAK ID, 3*25 cm, 5 m; Mobile Phase A: MTBE (10 mM NH₃-MeOH), Mobile Phase B: MeOH; Flow rate: 40 mL/min; Gradient: isocratic 10; Wave Length: 222/260 nm; RT1 (min): 8.069; RT2 (min): 10.72; Sample Solvent: EtOH: DCM=1:1) to afford (R)-1-((1-((2-hydroxyethyl)(methyl)amino)cyclopropyl)ethynyl)-4-((1-methyl-1H-pyrazol-4-yl)methyl-d2)-N-(1-methylcyclopropyl)-5-oxo-1,2,4,5-tetrahy droimidazo[1,2-a]quinazoline-7-sulfonamide (7.8 mg, 39%), (S)-1-((1-((2-hydroxyethyl)(methyl)amino)cyclopropyl)ethynyl)-4-((1-methyl-1H-pyrazol-4-yl)methyl-d2)-N-(1-methylcyclopropyl)-5-oxo-1,2,4,5-tetrahydroimidazo[1,2-a]quinazoline-7-sulfonamide (5.0 mg, 25%) both as white solids. LCMS (ESI) m/z: 554.25 [M+H]⁺. ¹H NMR (400 MHz, DMSO-d₆) δ 8.27 (d, J=2.2 Hz, 1H), 8.05 (s, 1H), 8.00 (dd, J=8.8, 2.2 Hz, 1H), 7.68 (s, 1H), 7.41 (d, J=0.8 Hz, 1H), 7.31 (d, J=8.8 Hz, 1H), 5.45-5.41 (m, 1H), 4.30-4.21 (m, 2H), 3.87-3.82 (m, 1H), 3.76 (s, 3H), 3.40-3.35 (m, 2H), 2.46 (t, J=6.2 Hz, 2H), 2.13 (s, 3H), 1.05 (s, 3H), 0.83-0.78 (m, 4H), 0.59 (q, J=4.6 Hz, 2H), 0.40-0.35 (m, 2H).

Representative Synthesis Route 45: Example 247: (R)-4-((2-(cyanomethyl)pyridin-3-yl)methyl)-1-methyl-N-(1-methylcyclopropyl)-5-oxo-1,2,4,5-tetrahydroimidazo[1,2-a]quinazoline-7-sulfonamide

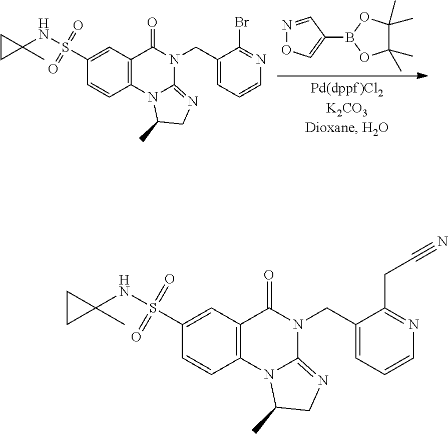

A mixture of (1R)-4-[(2-bromopyridin-3-yl)methyl]-1-methyl-N-(1-methylcyclopropyl)-5-oxo-1H,2H-imidazo[1,2-a]quinazoline-7-sulfonamide (150 mg, 0.3 mmol), 4-(4,4,5,5-tetramethyl-1,3,2-dioxaborolan-2-yl)-1,2-oxazole (116 mg, 0.59 mmol), Pd(dppf)Cl₂ (21.8 mg, 0.03 mmol) and K₂CO₃ (123.3 mg, 0.89 mmol) in dioxane (5 mL) and H₂O (0.5 mL) was stirred at 90° C. for 16 h under nitrogen atmosphere. The reaction was quenched by the addition of water (50 mL) at room temperature. The resulting mixture was extracted with EtOAc (2×50 mL). The combined organic layers were washed with brine (10 mL), dried over anhydrous Na₂SO₄. After filtration, the filtrate was concentrated under reduced pressure. The crude product (150 mg) was purified by Prep-HPLC with the following conditions (Column: Xselect CSH Prep C18, 30*150 mm 5 m; Mobile Phase A: Water (0.1% FA), Mobile Phase B: CH₃CN; Flow rate: 60 mL/min mL/min; Gradient: isocratic 7% to 25% B in 13 min; Wave Length: 254 nm/220 nm; RT1 (min): 12.68 min) to afford (R)-4-((2-(cyanomethyl)pyridin-3-yl)methyl)-1-methyl-N-(1-methylcyclopropyl)-5-oxo-1,2,4,5-tetrahydroimidazo[1,2-a]quinazoline-7-sulfonamide (20.2 mg, 15%) as a white solid. LCMS (ESI) m/z: 465 [M+H]⁺. ¹H NMR (400 MHz, DMSO-d₆) δ 8.47 (dd, J=4.8, 1.6 Hz, 1H), 8.24 (d, J=2.2 Hz, 1H), 8.06 (s, 1H), 7.94 (dd, J=8.8, 2.2 Hz, 1H), 7.72 (dd, J=7.8, 1.6 Hz, 1H), 7.36-7.26 (m, 2H), 5.19-5.05 (m, 2H), 4.77-4.74 (m, 1H), 4.50 (s, 2H), 4.00 (dd, J=13.6, 9.4 Hz, 1H), 3.46 (dd, J=13.6, 3.6 Hz, 1H), 1.34 (d, J=6.2 Hz, 3H), 1.09 (s, 3H), 0.64-0.57 (m, 2H), 0.42-0.35 (m, 2H).

Representative Synthesis Route 46: Example 254: (R)-1-methyl-N-(1-methylcyclopropyl)-5-oxo-4-((6-vinylpyridine-3-yl)methyl)-1,2,4,5-tetrahydroimidazo[1,2-a]quinazoline-7-sulfonamide

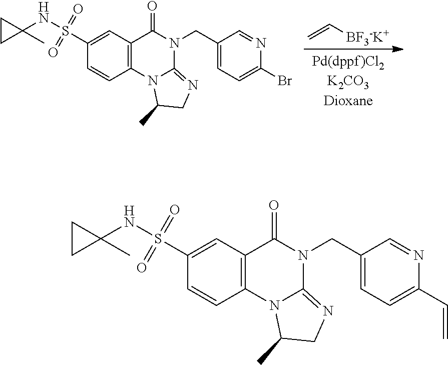

To a solution of (R)-4-((6-bromopyridin-3-yl)methyl)-1-methyl-N-(1-methylcyclopropyl)-5-oxo-1,2,4,5-tetrahydroimidazo[1,2-a]quinazoline-7-sulfonamide (46 mg, 0.09 mmol) in dioxane (0.3 mL) added potassium vinyltrifluoroborate (18.3 mg, 0.14 mmol) and K₂CO₃(37.8 mg, 0.27 mmol). The mixture was purged with nitrogen for 5 minutes and added Pd(dppf)Cl₂ (6.6 mg, 0.009 mmol). The reaction was heated to 100° C. and stirred for 1 hour.

The solution was cooled before being diluted with DMSO and isolated via prep -HPLC to afford (R)-1-methyl-N-(1-methylcyclopropyl)-5-oxo-4-((6-vinylpyridin-3-yl)methyl)-1,2,4,5-tetrahydroimidazo[1,2-a]quinazoline-7-sulfonamide as a tan solid (6 mg, 15%). LCMS (ESI) m/z: 452.00 [M+H]⁺. ¹H NMR (400 MHz, DMSO-d₆) δ 8.59 (d, J=2.0 Hz, 1H), 8.23 (d, J=2.2 Hz, 1H), 8.04 (s, 1H), 7.92 (dd, J=8.7, 2.3 Hz, 1H), 7.77 (dd, J=8.1, 2.3 Hz, 1H), 7.45 (d, J=8.1 Hz, 1H), 7.26 (d, J=8.7 Hz, 1H), 6.78 (dd, J=8.2, 7.4 Hz, 1H), 6.19 (dd, J=7.4, 1.6 Hz, 1H), 5.44 (dd, J=10.7, 3.6 Hz, 1H), 5.16-5.07 (m, 2H), 4.75-4.71 (m, 1H), 4.03 (dd, J=13.8, 9.3 Hz, 1H), 3.49 (dd, J=13.7, 3.6 Hz, 1H), 1.32 (d, J=6.2 Hz, 3H), 1.07 (s, 3H), 0.61-0.59 (m, 2H), 0.39-0.36 (m, 2H).

The following compounds in Table 24 were prepared using procedures similar to those described in Representative Synthesis Route 46 for Example 254 using appropriate starting materials.

TABLE 24

| Example | | |
|---|---|---|
| No. | Name | [M + H]⁺ |
| 255 | (R)-1-methyl-N-(1-methylcyclopropyl)-5-oxo-4-((2- | 452.00 |
| | vinylpyridin-4-yl)methyl)-1,2,4,5-tetrahydroimidazo[1,2- | |
| | a]quinazoline-7-sulfonamide | |

Representative Synthesis Route 47: Example 267: (R,E)-4-((2-(3-(dimethylamino)prop-1-en-1-yl)pyridin-4-yl)methyl)-1-methyl-N-(1-methylcyclopropyl)-5-oxo-1,2,4,5-tetrahydroimidazo[1,2-a]quinazoline-7-sulfonamide

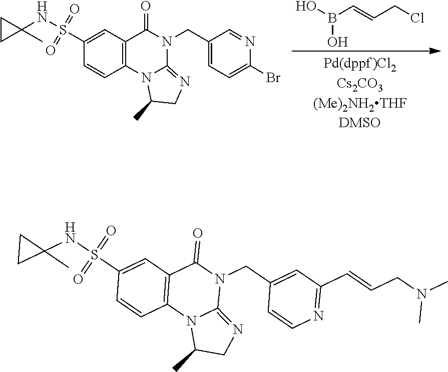 [SMILES: C[C@@H]1CN=C2N(Cc3ccc(Br)nc3)C(=O)c3cc(S(=O)(=O)NC4(C)CC4)ccc3N21 ; OB(O)/C=C/CCl ; C[C@@H]1CN=C2N(Cc3ccnc(/C=C/CN(C)C)c3)C(=O)c3cc(S(=O)(=O)NC4(C)CC4)ccc3N21]

To a solution of (R)-4-((6-bromopyridin-3-yl)methyl)-1-methyl-N-(1-methylcyclopropyl)-5-oxo-1,2,4,5-tetrahydroimidazo[1,2-a]quinazoline-7-sulfonamide (25 mg, 0.05 mmol) in DMSO (0.3 mL) added [(E)-3-chloro-1-propenyl]boranediol (11.9 mg, 0.1 mmol), Cs₂CO₃ (32.3 mg, 0.1 mmol). The reaction was purged with nitrogen for 5 minutes. Subsequently added Pd(dppf)Cl₂ (7.3 mg, 0.01 mmol) and 2M dimethylamine in THF (0.05 mL, 0.1 mmol) and the reaction was heated to 100° C. and stirred for 1 h. The solution was cooled and was diluted with DMSO and purified by prep-HPLC to afford (R,E)-4-((2-(3-(dimethylamino)prop-1-en-1-yl)pyridin-4-yl)methyl)-1-methyl-N-(1-methylcyclopropyl)-5-oxo-1,2,4,5-tetrahydroimidazo[1,2-a]quinazoline-7-sulfonamide as a tan solid (5.5 mg, 22%). LCMS (ESI) m/z: 509.00 [M+H]⁺. ¹HNMR (400 MHz, DMSO-d₆) δ 8.42 (d, J=5.0 Hz, 1H), 8.24 (dd, J=6.1, 2.2 Hz, 1H), 8.05 (s, 1H), 8.00-7.93 (m, 1H), 7.36-7.34 (m, 1H), 7.30-7.27 (m, 1H), 7.16-7.13 (m, 1H), 6.72-6.59 (m, 2H), 5.15-5.07 (m, 2H), 4.76-4.73 (m, 1H), 3.99 (td, J=9.5, 1.9 Hz, 1H), 3.51-3.43 (m, 2H), 3.09 (d, J=2.2 Hz, 1H), 2.19 (s, 3H), 1.34 (t, J=Hz, 3H), 1.26-1.23 (m, 2H), 1.09 (s, 3H), 0.61-0.58 (m, 2H), 0.39-0.35 (m, 2H).

Representative Synthesis Route 48: Example 278: (R)-4-(3-(6-fluoropyridin-3-yl)prop-2-yn-1-yl)-1-methyl-N-(1-methylcyclopropyl)-5-oxo-1,2,4,5-tetrahy droimidazo[1,2-a]quinazoline-7-sulfonamide

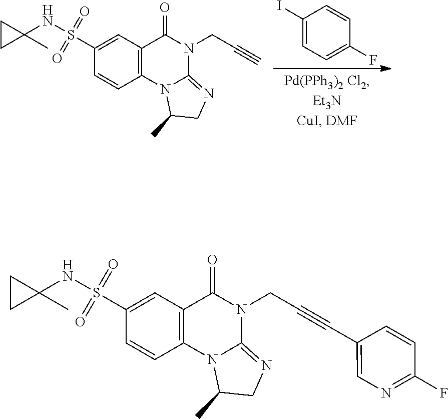 [SMILES: C#CCN1C(=O)c2cc(S(=O)(=O)NC3(C)CC3)ccc2N2C1=NC[C@H]2C ; Fc1ccc(I)cc1 ; C[C@@H]1CN=C2N(CC#Cc3ccc(F)nc3)C(=O)c3cc(S(=O)(=O)NC4(C)CC4)ccc3N21]

A mixture of (1R)-1-methyl-N-(1-methylcyclopropyl)-5-oxo-4-(prop-2-yn-1-yl)-1H,2H-imidazo[1,2-a]quinazoline-7-sulfonamide(150 mg, 0.4 mmol), 2-fluoro-5-iodopyridine (107.8 mg, 0.48 mmol), Et₃N (203.8 mg, 2.01 mmol), CuI (15.3 mg, 0.081 mmol) and Pd(PPh₃)₂Cl₂ (28.3 mg, 0.04 mmol) in DMF (1 mL) was stirred at 80° C. for 16 h under nitrogen atmosphere. The reaction was quenched by the addition of water (30 mL) at room temperature. The resulting mixture was extracted with EtOAc (2×20 mL). The combined organic layers were washed with brine (2×10 mL), dried over anhydrous Na₂SO₄. After filtration, the filtrate was concentrated under reduced pressure. The crude product (150 mg) was purified by Prep-HPLC with the following conditions (Column: XSelect CSH Fluoro Phenyl 30*150 mm, 5 m; Mobile Phase A: Water (0.1% FA), Mobile Phase B: CH₃CN; Flow rate: 60 mL/min; Gradient (B %): 11% B to 28% B in 30 min; Wave Length: 254 nm/220 nm; RT1 (min): 11.8 min) to afford (R)-4-(3-(6-fluoropyridin-3-yl)prop-2-yn-1-yl)-1-methyl-N-(1-methylcyclopropyl)-5-oxo-1,2,4,5-tetrahydroimidazo[1,2-a]quinazoline-7-sulfonamide (6.8 mg, 4%) as a white solid. LCMS (ESI) m/z: 468.25 [M+H]⁺. 1H NMR (400 MHz, DMSO-d₆) δ 8.34 (d, J=2.4 Hz, 1H), 8.29 (d, J=2.2 Hz, 1H), 8.16-8.02 (m, 2H), 7.98 (dd, J=8.7, 2.2 Hz, 1H), 7.34 (d, J=8.8 Hz, 1H), 7.22 (dd, J=8.6, 2.8 Hz, 1H), 5.05-4.89 (m, 2H), 4.82 (s, 1H), 4.09 (dd, J=13.4, 9.4 Hz, 1H), 3.56 (dd, J=13.4, 3.6 Hz, 1H), 1.37 (d, J=6.2 Hz, 3H), 1.09 (s, 3H), 0.64-0.56 (m, 2H), 0.43-0.37 (m, 2H).

Representative Synthesis Route 49: Example 279: (R)-4-((5-acetyl-4,5,6,7-tetrahydropyrazolo[1,5-a]pyrazin-3-yl)methyl)-1-methyl-N-(1-methylcyclopropyl)-5-oxo-1,2,4,5-tetrahydroimidazo[1,2-a]quinazoline-7-sulfonamide

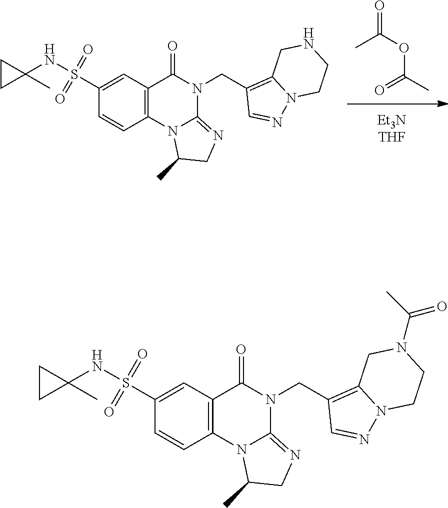

To a solution (R)-1-methyl-N-(1-methylcyclopropyl)-5-oxo-4-((4,5,6,7-tetrahydropyrazolo[1,5-a]pyrazin-3-yl)methyl)-1,2,4,5-tetrahydroimidazo[1,2-a]quinazoline-7-sulfonamide (5.2 mg, 0.01 1 mmol) in THF (1 mL) added Ac₂O (1 uL, 0.011 mmol) and Et₃N (4.6 uL, 0.033 mmol) and the reaction was stirred at room temperature for 30 min. The solvent was removed in vacuo and the crude was purified by silica gel chromatography (0-100% Hex: [25% EtOH in EtOAc+1% TEA]) to afford (R)-4-((5-acetyl-4,5,6,7-tetrahydropyrazolo[1,5-a]pyrazin-3-yl)methyl)-1-methyl-N-(1-methylcyclopropyl)-5-oxo-1,2,4,5-tetrahydroimidazo[1,2-a]quinazoline-7-sulfonamide, as a white solid (6.4 mg, 94%). LCMS (ESI) m/z: 512.20 [M+H]⁺ 0.1H NMR (400 MHz, DMSO-d₆) δ 8.24 (dd, J=5.3, 2.2 Hz, 1H), 8.03 (d, J=3.4 Hz, 1H), 7.90 (dd, J=8.6, 2.1 Hz, 1H), 7.48 (d, J=5.4 Hz, 1H), 7.23 (dd, J=8.8, 3.3 Hz, 1H), 4.87-4.84 (m, 2H), 4.72-4.70 (m, 1H), 4.12-4.00 (m, 3H), 3.88-3.86 (m, 2H), 3.56-3.52 (m, 1H), 3.10-3.06 (m, 2H), 2.12 (d, J=4.0 Hz, 3H), 1.32 (d, J=4.0 Hz, 3H), 1.06 (s, 3H), 0.60-0.58 (m, 2H) 0.38-0.36 (m, 2H).

The following compounds in Table 25 were prepared using procedures similar to those described in Representative Synthesis Route 49 for Example 279 using appropriate starting materials.

TABLE 25

| Example | | |
|---|---|---|
| No. | Name | [M + H]⁺ |
| 291 | (7aS,10aR)-6-((5-acetyl-4,5,6,7-tetrahydropyrazolo[1,5- | 538.00 |
| | a]pyrazin-3-yl)methyl)-N-(1-methylcyclopropyl)-5-oxo- | |
| | 6,7a,8,9,10,10a-hexahydro-5H-cyclopenta[4,5]imidazo[1,2- | |
| | a]quinazoline-3-sulfonamide | |
| 292 | (R)-9-(4-acetylpiperazin-1-yl)-1-methyl-4-((1-methyl-1H- | 555.30 |
| | pyrazol-4-yl)methyl)-N-(1-methylcyclopropyl)-5-oxo-1,2,4,5- | |
| | tetrahydroimidazo[1,2-a]quinazoline-7-sulfonamide | |
| 295 | N-(((1R,2S)-1-methyl-4-((1-methyl-1H-pyrazol-4-yl)methyl)- | 500.20 |
| | 7-(N-(1-methylcyclopropyl)sulfamoyl)-5-oxo-1,2,4,5- | |
| | tetrahydroimidazo[1,2-a]quinazolin-2-yl)methyl)acetamide | |

Representative Synthesis Route 50: Example 285: (7aR,9R,10aS)-9-(dimethylamino)-6-((1-methyl-1H-pyrazol-4-yl)methyl-d2)-N-(1-methylcyclopropyl)-5-oxo-6,7a,8,9,10,1Oa-hexahydro-5H-cyclopenta[4,5]imidazo[1,2-a]quinazoline-3-sulfonamide

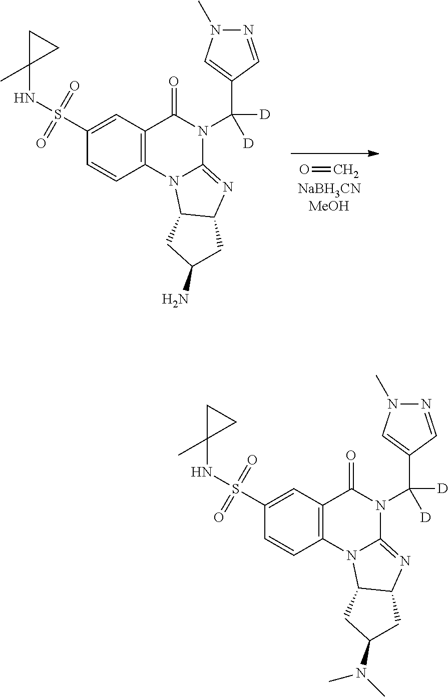

A solution of (7aR,9R,10aS)-9-amino-6-((1-methyl-1H-pyrazol-4-yl)methyl-d2)-N-(1-methylcyclopropyl)-5-oxo-6,7a,8,9,10,10a-hexahydro-5H-cyclopenta[4,5]imidazo[1,2-a]quinazoline-3-sulfonamide (100 mg, 0.21 mmol) in methanol (5 mL) was treated with formaldehyde (12.7 mg, 0.42 mmol) for 30 min at room temperature under nitrogen atmosphere followed by the addition of sodium cyanoborohydride (26.6 mg, 0.42 mmol) in portions at 0° C. for 30 mins. The resulting mixture was stirred for 2 h at room temperature under nitrogen atmosphere. The reaction was quenched by the addition of water (30 mL) at room temperature. The resulting mixture was extracted with CH₂Cl₂ (3×100 mL). The combined organic layers were washed with brine (2×100 mL), dried over anhydrous MgSO₄. After filtration, the filtrate was concentrated under reduced pressure. The crude product (70 mg) was purified by Prep-HPLC with the following conditions (Column: XBridge Prep OBD C18 Column, 30*150 mm, 5 m; Mobile Phase A: Water (10 mmol/L NH₄HCO₃), Mobile Phase B: ACN; Flow rate: 60 mL/min mL/min; Gradient: 12% B to 42% B in 10 min; Wave Length: 254 nm/220 nm; RT1 (min): 8.4) to afford (7aR,9R,10aS)-9-(dimethylamino)-6-((1-methyl-1H-pyrazol-4-yl)methyl-d2)-N-(1-methylcyclopropyl)-5-oxo-6,7a,8,9,10,10a-hexahydro-5H-cyclopenta[4,5]imidazo[1,2-a]quinazoline-3-sulfonamide (29.3 mg, 28%) as a white solid. LCMS (ESI) m/z: 500.25 [M+H]⁺. ¹HNMR (400 MHz, DMSO-d₆) δ 8.22 (d, J=2.0 Hz, 1H), 8.03 (s, 1H), 7.91 (dd, J=8.6, 2.4 Hz, 1H), 7.69 (s, 1H), 7.42 (s, 1H), 7.12 (d, J=8.8 Hz, 1H), 4.87 (t, J=7.6 Hz, 1H), 4.65 (t, J=7.6 Hz, 1H), 3.76 (s, 3H), 2.47-2.43 (m, 1H), 2.16-2.06 (m, 8H), 1.78-1.68 (m, 2H), 1.08 (s, 3H), 0.60-0.56 (m, 2H), 0.42-0.33 (m, 2H).

The following compounds in Table 26 were prepared using procedures similar to those described in Representative Synthesis Route 50 for Example 285 using appropriate starting materials.

TABLE 26

| Example | | |
|---|---|---|
| No. | Name | [M + H]⁺ |
| 284 | (7aS,9S,10aR)-9-(dimethylamino)-6-((1-methyl-1H-pyrazol-4- | 500.25 |
| | yl)methyl-d2)-N-(1-methylcyclopropyl)-5-oxo- | |
| | 6,7a,8,9,10,10a-hexahydro-5H-cyclopenta[4,5]imidazo[1,2- | |
| | a]quinazoline-3-sulfonamide | |

Representative Synthesis Route 51: Example 287: (7aS,10aR)-6-((1-methyl-1H-pyrazol-4-yl)methyl-d2)-N-(1-methylcyclopropyl)-5-oxo-2-((3-(pyrimidin-5-yl)prop-2-yn-1-yl)amino)-6,7a,8,9,10,1 Oa-hexahydro-5H-cyclopenta[4,5]imidazo[1,2-a]quinazoline-3-sulfonamide

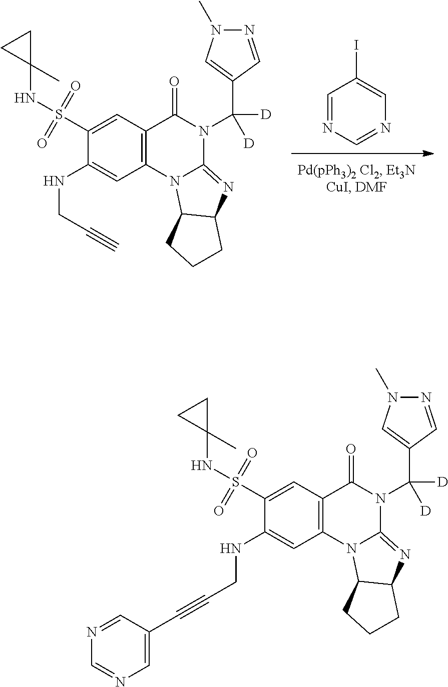 [SMILES: [2H]C([2H])(c1cnn(C)c1)N1C(=O)c2cc(S(=O)(=O)NC3(C)CC3)c(NCC#C)cc2N2C1=N[C@H]1CCC[C@H]12 ; Ic1cncnc1 ; [2H]C([2H])(c1cnn(C)c1)N1C(=O)c2cc(S(=O)(=O)NC3(C)CC3)c(NCC#Cc3cncnc3)cc2N2C1=N[C@H]1CCC[C@H]12]

To a solution of (7aS,10aR)-6-((1-methyl-1H-pyrazol-4-yl)methyl-d2)-N-(1-methylcyclopropyl)-5-oxo-2-(prop-2-yn-1-ylamino)-6,7a,8,9,10,10a-hexahydro-5H-cyclopenta[4,5]imidazo[1,2-a]quinazoline-3-sulfonamide (50 mg, 0.098 mmol), 5-iodopyrimidine (20.2 mg, 0.098 mmol) in DMF (1 mL) added Et₃N (41 uL, 0.29 mmol), Pd(PPh₃)₂Cl₂ (7 mg, 0.0098 mmol), degassed and back filled with N₂ three times. Subsequently added CuI (3 mg) and degassed and back filled with N₂ three times. The reaction was heated at 90° C. for 1 h, diluted with DMSO (1 mL). To this mixture added SiliaMetS Thiol (50 mg) and SiliaMetS TAAcONa (50 mg) and stirred for 1 h. The reaction mixture was filtered and purified by prep-HPLC to obtain (7aS,10aR)-6-((1-methyl-1H-pyrazol-4-yl)methyl-d2)-N-(1-methylcyclopropyl)-5-oxo-2-((3-(pyrimidin-5-yl)prop-2-yn-1-yl)amino)-6,7a,8,9,10,10a-hexahydro-5H-cyclopenta[4,5]imidazo[1,2-a]quinazoline-3-sulfonamide as a tan solid (4 mg, 7%). LCMS (ESI) m/z: 588.20 [M+H]⁺. ¹H NMR (400 MHz, DMSO-d₆) 6 9.17 (s, 1H), 8.83 (s, 2H), 8.16 (s, 1H) 8.06 (s, 1H), 7.65 (s, 1H), 7.39 (s, 1H), 6.82-6.80 (m, 1H), 6.19 (s, 1H), 4.79-4.77 (m, 1H), 4.63-4.62 (m, 1H), 4.56 (t, J=4.0 Hz, 2H), 3.75 (s, 3H), 1.85-1.83 (m, 4H) 1.41-1.39 (m, 2H), 1.07 (s, 3H), 0.66-0.64 (m, 2H) 0.37-0.35 (m, 2H).

Representative Synthesis Route 52: Example 295: Methyl (1R,2R)-1-methyl-4-((1-methyl-1H-pyrazol-4-yl)methyl)-7-(N-(1-methylcyclopropyl)sulfamoyl)-5-oxo-1,2,4,5-tetrahy droimidazo[1,2-a]quinazoline-2-carboxylate

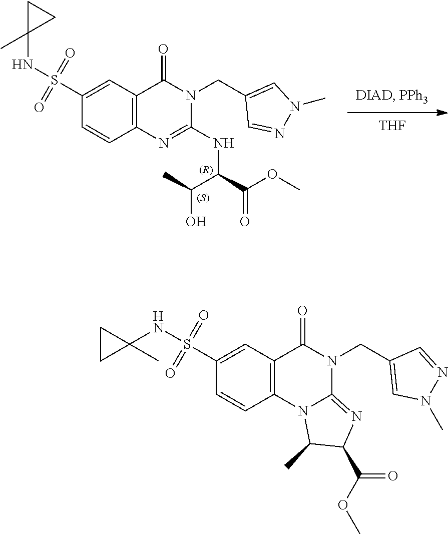

A mixture of methyl (2R,3S)-3-hydroxy-2-({6-[(1-methylcyclopropyl)sulfamoyl]-3-[(1-methylpyrazol-4-yl)methyl]-4-oxoquinazolin-2-yl}amino)butanoate (50 mg, 0.099 mmol) and PPh₃ (39 mg, 0.15 mmol) in THF (1 mL) was stirred at room temperature for 5 min under nitrogen atmosphere. To the above mixture was added DIAD (30.1 mg, 0.15 mmol) dropwise over 2 min at 0° C. The resulting mixture was stirred at room temperature for additional 3 h. The resulting mixture was concentrated under vacuum. The residue was purified by silica gel column chromatography, eluted with PE/EA (1:1) to afford the crude product (20 mg) as a white solid.

The residue was purified by HPLC with the following conditions: Column: Xselect CSH Prep C18, 30*150 mm 5 m; Mobile Phase A: Water (0.1% FA), Mobile Phase B: MeOH: HPLC; Flow rate: 60 mL/min; Gradient (B %): isocratic 42% to 63% B in 12 min; Wave Length: 254 nm/220 nm; RTl(min): 10.78 min. This resulted in the desired product (4.7 mg, 10%) as a white solid. LCMS (ESI) m/z: 487.15 [M+H]⁺. ¹H NMR (400 MHz, DMSO-d₆) δ 8.46 (d, J=2.2 Hz, 1H), 8.07 (dd, J=8.6, 2.2 Hz, 1H), 7.72-7.65 (m, 2H), 7.46 (s, 1H), 5.18-5.14 (m, 2H), 3.76-3.73 (m, 6H), 3.33 (dd, J=7.1, 5.6 Hz, 1H), 1.37 (d, J=5.6 Hz, 3H), 1.05 (s, 3H), 0.63-0.55 (m, 2H), 0.43-0.36 (m, 2H).

Representative Synthesis Route 53: Example 296: (1R,2R)-2-(hydroxymethyl)-1-methyl-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

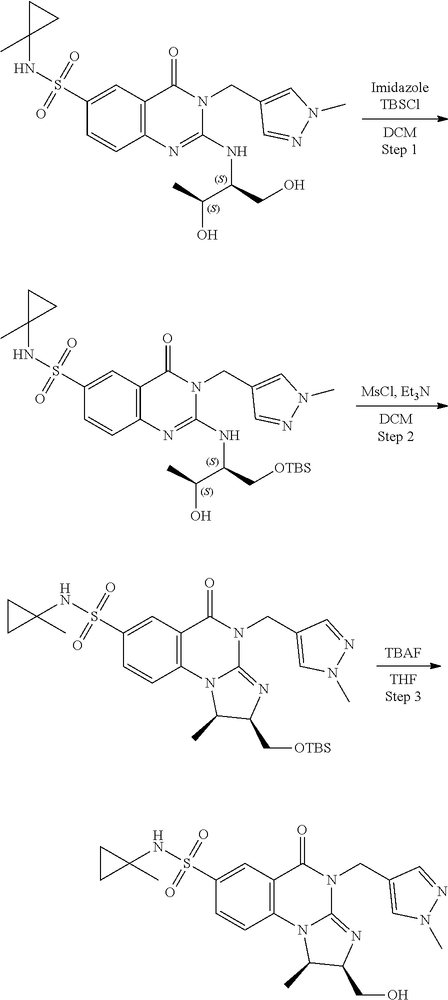

Step 1: A solution of 2-{[(2S,3 S)-1,3-dihydroxybutan-2-yl]amino}-N-(1-methylcyclopropyl)-3-[(1-methylpyrazol-4-yl)methyl]-4-oxoquinazoline-6-sulfonamide (460 mg, 0.96 mmol), imidazole (197.1 mg, 2.89 mmol) and TBSCl (145.5 mg, 0.96 mmol) in DCM (20 mL) was stirred at room temperature for 1 h. Desired product was detected by LCMS. The reaction was quenched with water at room temperature. The resulting mixture was extracted with EtOAc (3×100 mL). The combined organic layers were dried over anhydrous Na₂SO₄. After filtration, the filtrate was concentrated under reduced pressure. The residue was purified by silica gel column chromatography, with PE/EA (4:5) to afford 2-{[(2S,3S)-1-[(tert-butyldimethylsilyl)oxy]-3-hydroxybutan-2-yl]amino}-N-(1-methylcyclopropyl)-3-[(1-methylpyrazol-4-yl)methyl]-4-oxoquinazoline-6-sulfonamide (498 mg) as a light yellow solid. LCMS (ESI) m/z: 591 [M+H]⁺.

Step 2: A solution of 2-{[(2S,3 S)-1-[(tert-butyldimethylsilyl)oxy]-3-hydroxybutan-2-yl]amino}-N-(1-methylcyclopropyl)-3-[(1-methylpyrazol-4-yl)methyl]-4-oxoquinazoline-6-sulfonamide (440 mg, 0.74 mmol) and Et₃N (226.1 mg, 2.23 mmol) in DCM (10 mL) was stirred at room temperature for 1 min. To the above mixture was added methanesulfonyl chloride (426.5 mg, 3.72 mmol) dropwise at 0° C. The resulting mixture was stirred at 40° C. overnight. Desired product was detected by LCMS. The resulting mixture was concentrated under reduced pressure. The resulting mixture was diluted with water (50 mL) and extracted with EtOAc (3×50 mL). The combined organic layers were dried over anhydrous Na₂SO₄. After filtration, the filtrate was concentrated under reduced pressure. The residue was purified by silica gel column chromatography, PE/EA (1:9) to afford (1R,2R)-2-{[(tert-butyldimethylsilyl)oxy]methyl}-1-methyl-N-(1-methylcyclopropyl)-4-[(1-methylpyrazol-4-yl)methyl]-5-oxo-1H,2H-imidazo[1,2-a]quinazoline-7-sulfonamide (350 mg, 82%) as a light yellow solid. LCMS (ESI) m/z: 573 [M+H]⁺.

Step 3: A solution of (1R,2R)-2-{[(tert-butyldimethylsilyl)oxy]methyl}-1-methyl-N-(1-methylcyclopropyl)-4-[(1-methylpyrazol-4-yl)methyl]-5-oxo-1H,2H-imidazo[1,2-a]quinazoline-7-sulfonamide (50 mg, 0.087 mmol) and TBAF (45.6 mg, 0.17 mmol) in THF (1 mL) was stirred at room temperature for 1 h. Desired product was be detected by LCMS. The resulting mixture was concentrated under vacuum. The resulting mixture was diluted with water (20 mL) and extracted with EtOAc (3×20 mL). The combined organic layers were dried over anhydrous Na₂SO₄. After filtration, the filtrate was concentrated under reduced pressure. The residue was purified by silica gel column chromatography, eluted with CH₂Cl₂/MeOH (9:1) to afford the crude as a light yellow solid. The crude product (30 mg) was purified by Prep-HPLC with the following conditions (Column: XBridge Shield RP18 OBD Column 30*150 mm, 5 m; Mobile Phase A: Water (10 mmol/L NH₄HCO₃), Mobile Phase B: ACN; Flow rate: 60 mL/min; Gradient (B %): 17% B to 30% B in 17 min; Wave Length: 254 nm/220 nm; RT1(min): 9.382 min) to afford the desired product (12.5 mg, 31%) as a white solid. LCMS (ESI) m/z: 459.20 [M+H]⁺. ¹HNMR (400 MHz, DMSO-d₆) δ 8.23 (d, J=2.4 Hz, 1H), 8.04 (s, 1H), 7.91 (dd, J=8.8, 2.4 Hz, 1H), 7.67 (s, 1H), 7.41 (d, J=0.8 Hz, 1H), 7.29 (d, J=8.8 Hz, 1H), 4.96-4.76 (m, 4H), 4.24-4.16 (m, 1H), 3.90-3.82 (m, 1H), 3.76 (s, 3H), 3.66 (td, J=10.2, 6.0 Hz, 1H), 1.24 (d, J=6.4 Hz, 3H), 1.06 (s, 3H), 0.60-0.59 (m, 2H), 0.40-0.35 (m, 2H).

Representative Synthesis Route 54: Example 298: (1R,2S)-2-(aminomethyl)-1-methyl-4-((1-methyl-1H-pyrazol-4-yl)methyl)-N-(1-methylcyclopropyl)-5-oxo-1,2,4,5-tetrahydroimidazo[1,2-a]quinazoline-7-sulfonamide

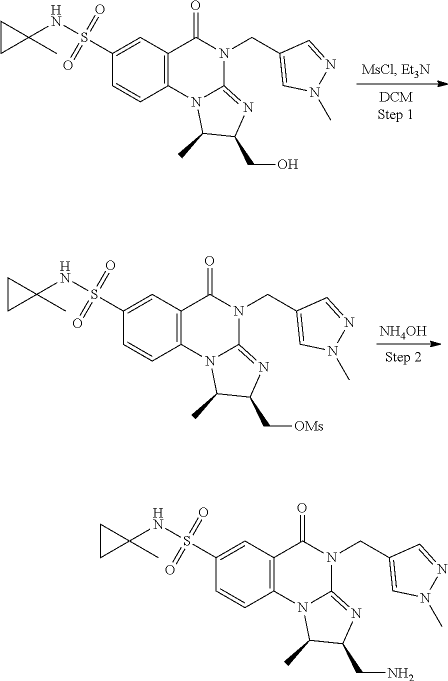 [SMILES: C[C@@H]1[C@H](CO)N=C2N(Cc3cnn(C)c3)C(=O)c3cc(S(=O)(=O)NC4(C)CC4)ccc3N21 ; "DCMStep 1" ; C[C@@H]1[C@H](COS(C)(=O)=O)N=C2N(Cc3cnn(C)c3)C(=O)c3cc(S(=O)(=O)NC4(C)CC4)ccc3N21 ; "NH4OH" ; "Step 2" ; C[C@@H]1[C@H](CN)N=C2N(Cc3cnn(C)c3)C(=O)c3cc(S(=O)(=O)NC4(C)CC4)ccc3N21]

Step 1: A solution of (1R,2R)-2-(hydroxymethyl)-1-methyl-N-(1-methylcyclopropyl)-4-[(1-methylpyrazol-4-yl)methyl]-5-oxo-1H,2H-imidazo[1,2-a]quinazoline-7-sulfonamide (200 mg, 0.44 mmol) and Et₃N (132.4 mg, 1.31 mmol) in DCM (5 mL) was stirred at room temperature for 1 min. To the above mixture was added methanesulfonyl chloride (149.9 mg, 1.31 mmol) dropwise at 0° C. The resulting mixture was stirred at room temperature for additional 1 h.

Desired product was detected by LCMS. The resulting mixture was diluted with water (50 mL) and extracted with EtOAc (3×50 mL). The combined organic layers were dried over anhydrous Na₂SO₄. After filtration, the filtrate was concentrated under reduced pressure. This resulted in [(1R,2R)-1-methyl-7-[(1-methylcyclopropyl)sulfamoyl]-4-[(1-methylpyrazol-4-yl)methyl]-5-oxo-1H,2H-imidazo[1,2-a]quinazolin-2-yl]methyl methanesulfonate (280 mg) as a light yellow solid. The crude product was used in the next step directly without further purification. LCMS (ESI) m/z: 537 [M+H]⁺.

Step 2: A solution of [(1R,2R)-1-methyl-7-[(1-methylcyclopropyl)sulfamoyl]-4-[(1-methylpyrazol-4-yl)methyl]-5-oxo-1H,2H-imidazo[1,2-a]quinazolin-2-yl]methyl methanesulfonate (130 mg, 0.24 mmol) in ammonium hydroxide (6 mL) was stirred at 100° C. for 2 h. Desired product was detected by LCMS. The resulting mixture was concentrated under reduced pressure. The residue was purified by silica gel column chromatography, eluted with CH₂Cl₂/MeOH (4:1) to afford the crude as a white solid. The crude product (60 mg) was purified by Prep-HPLC with the following conditions (Column: XBridge Shield RP18 OBD Column 30*150 mm, 5 m; Mobile Phase A: Water (10 mmol/L NH₄HCO₃), Mobile Phase B: ACN; Flow rate: 60 mL/min; Gradient (B %): 17% B to 32% B in 17 min; Wave Length: 254 nm/220 nm; RT1 (min): 7.809 min) to afford the desired product (25.7 mg, 23%) as a white solid. LCMS (ESI) m/z: 458.15 [M+H]⁺. ¹HNMR (400 MHz, DMSO-d₆) δ 8.23 (d, J=2.4 Hz, 1H), 7.90 (dd, J=8.8, 2.0 Hz, 1H), 7.67 (s, 1H), 7.42 (s, 1H), 7.24 (d, J=8.8 Hz, 1H), 4.92-4.77 (m, 3H), 4.05 (q, J=7.6 Hz, 1H), 3.76 (s, 3H), 2.94 (dd, J=12.4, 7.2 Hz, 1H), 2.81 (dd, J=12.4, 7.2 Hz, 1H), 1.18 (d, J=6.4 Hz, 3H), 1.06 (s, 3H), 0.61-0.56 (m, 2H), 0.39-0.35 (m, 2H).

### Synthesis of Intermediates

### Intermediate 1: 7-1-amino-4-cyclopropylbut-3-yn-2-ol

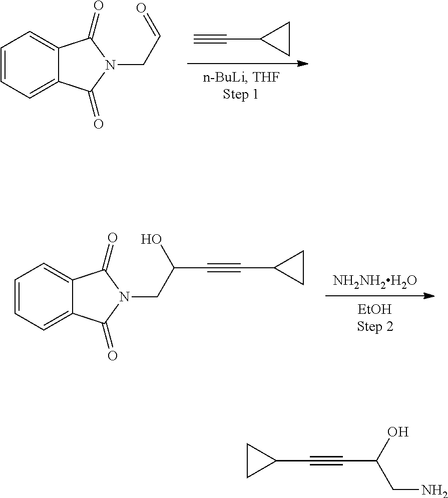

Step 1: To a stirred solution of ethynylcyclopropane (1 g, 15 mmol) in anhydrous THF (20 mL) was added n-BuLi (1.1 g, 16.6 mmol) at −78° C. for 30 min. To the above mixture was added Phthalimidoacetaldehyde (3.15 g, 16.6 mmol) dropwise over 15 min at −78° C. The resulting mixture was stirred for additional 2 h at room temperature. After completion of reaction, the reaction mixture was quenched by addition of saturated NH₄Cl solution (20 mL). The aqueous layer was extracted with ethyl acetate (300 mL). The combined organic phase was washed with brine (100 mL), dried over anhydrous sodium sulfate and concentrated under reduced pressure to give crude product which was further purified by silica gel column chromatography using 10 to 30% ethyl acetate in PE gradient to afford desired compound 2-(4-cyclopropyl-2-hydroxybut-3-yn-1-yl) isoindole-1,3-dione (0.8 g). LCMS (ESI) m/z: 256 [M+H]

Step 2: To a stirred solution of 2-(4-cyclopropyl-2-hydroxybut-3-yn-1-yl) isoindole-1,3-dione (800 mg, 3.1 mmol) in anhydrous EtOH (25 mL) was added hydrazine hydrate (307 mg, 6.3 mmol) at room temperature. The reaction mixture was stirred at 70° C. for 2 h. After completion of reaction, the reaction mixture was quenched by addition of water (10 mL). The aqueous layer was extracted with ethyl acetate (200 mL), dried over anhydrous sodium sulfate and concentrated under reduced pressure to give crude product which was further purified by column chromatography (10 to 30% MeOH/DCM) to afford desired compound 1-amino-4-cyclopropylbut-3-yn-2-ol (200 mg). LCMS (ESI) m/z: 126 [M+H]⁺.

Intermediate 1 was used for the synthesis of Examples 14, 15, 16 and 34. In a similar manner using appropriate starting material, tert-butyl 3-(4-amino-3-hydroxybut-1-yn-1-yl)azetidine-1-carboxylate was synthesized and used during the synthesis of Example 89. In a similar manner using appropriate starting material following intermediates were synthesized.

TABLE 27

| Example | |
|---|---|
| No. | Intermediate |
| 96 | 1-amino-6-methylhept-3-yn-2-ol |
| 105 | tert-butyl N-(6-amino-5-hydroxy-2-methylhex-3- |
| | yn-2-yl)carbamate |

### Intermediate 2: 1-amino-3-isopropoxypropan-2-ol

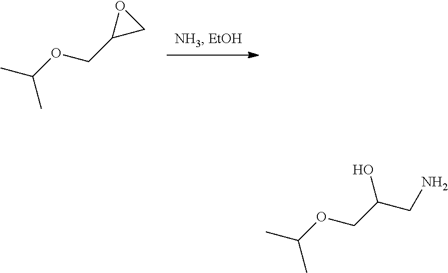

A solution of isopropyl glyceryl ether (1 g, 8.61 mmol) and NH₃ in EtOH (50 mL) was stirred for 2 h at room temperature. The residue was purified by reversed-phase flash chromatography with the following conditions: column, C18 silica gel; mobile phase, MeCN in water (0.1% FA), 10% to 50% gradient in 10 min; detector, UV 254 nm. This resulted in 1-amino-3-isopropoxypropan-2-ol (400 mg) as a white solid. LCMS (ESI) m/z: 134 [M+H]⁺.

Intermediate 2 was used for the synthesis of Example 19. In a similar manner using appropriate starting material following intermediates were synthesized.

TABLE 28

| Example | |
|---|---|
| No. | Intermediate |
| 28 | 1-amino-3-phenoxypropan-2-ol |
| 29 | 1-amino-3-(4-fluorophenoxy)propan-2-ol |
| 30 | 1-amino-3-(3-fluorophenoxy)propan-2-ol |
| 122 | 1-amino-2-pentanol |

### Intermediate 3: 4-(bromomethyl)-1-methyl-3-(trifluoromethyl)pyrazole

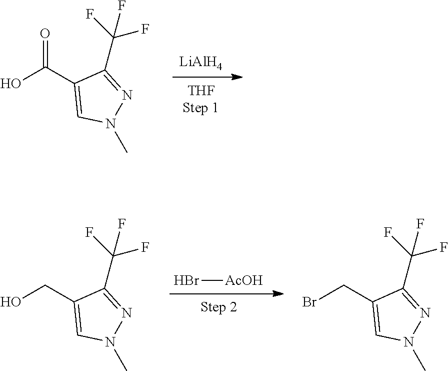

Step 1: To a stirred solution of 1-methyl-3-(trifluoromethyl)pyrazole-4-carboxylic acid (2 g, 10.3 mmol) in THF (40 mL) was added LiAlH₄ (0.78 g, 20.6 mmol) in portions at 0° C. The resulting mixture was stirred for 2 h at 0° C. under nitrogen atmosphere. The reaction was quenched by the addition of Na₂SO₄.10H₂O (3 g) at 0° C. The resulting mixture was filtered, the filter cake was washed with MeCN (3×20 mL). The filtrate was concentrated under reduced pressure to afford [1-methyl-3-(trifluoromethyl)pyrazol-4-yl]methanol (1.6 g, 86%) as a colorless oil. LCMS (ESI) m/z: 181 [M+H]⁺.

Step 2: A solution of [1-methyl-3-(trifluoromethyl)pyrazol-4-yl]methanol (800 mg, 4.44 mmol) in HBr in CH₃COOH (40%) (5 mL) was stirred for 16 h at 100° C. The resulting oil was dried under vacuum to afford 4-(bromomethyl)-1-methyl-3-(trifluoromethyl)pyrazole (850 mg, 79%) as a brown oil. LCMS (ESI) m/z: 243, 245 [M+H]⁺.

Intermediate 3 was used for the synthesis of Example 23. In a similar manner using appropriate starting material 5-(bromomethyl)-2-methyl-1,3-oxazole was synthesized, this was used for the synthesis of Example 153.

### Intermediate 4: 4-(bromomethyl)-3-(difluoromethyl)-1-methylpyrazole

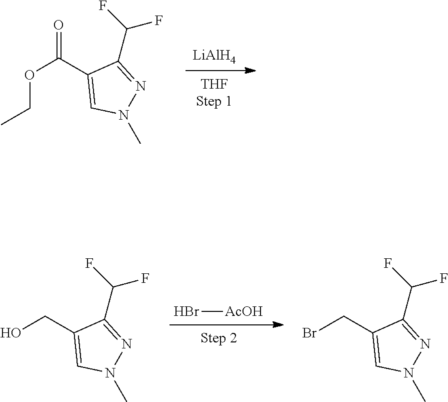

Step 1: A mixture of ethyl 3-(difluoromethyl)-1-methylpyrazole-4-carboxylate (1.2 g, 5.87 mmol) and LiAlH₄ (0.45 g, 11.7 mmol) in THF (15 mL) was stirred for 16 h at 0° C. The reaction was quenched by the addition of water (50 mL) at 0° C. The resulting mixture was filtered, the filter cake was washed with MeCN (3×30 mL). The filtrate was concentrated under reduced pressure. This resulted in [3-(difluoromethyl)-1-methylpyrazol-4-yl]methanol (800 mg) as a light brown oil, which was used in the next step directly without further purification. LCMS (ESI) m/z: 163 [M+H]⁺.

Step 2: A mixture of [3-(difluoromethyl)-1-methylpyrazol-4-yl]methanol (800 mg, 4.93 mmol) and HBr-AcOH (12 mL, 3 mmol) was stirred overnight at 110° C.. The resulting mixture was concentrated under reduced pressure and washed with Et₂O (50 mL). The resulting mixture was filtered, the filter cake was washed with Et₂O (3×50 mL). The filtrate was concentrated under reduced pressure to obtain 4-(bromomethyl)-3-(difluoromethyl)-1-methylpyrazole (800 mg) as a light brown solid, which was used in the next step directly without further purification. LCMS (ESI) m/z: 225, 227 [M+H]⁺.

Intermediate 4 was used for the synthesis of Example 24.

### Intermediate 5: 1-amino-5-(dimethylamino)pent-3-yn-2-ol

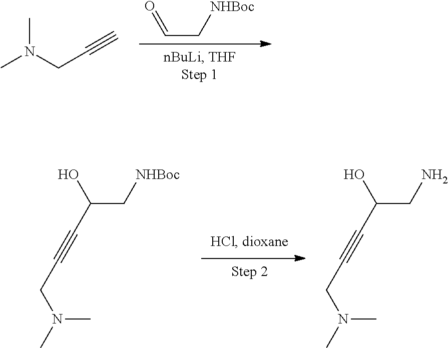

Step 1: In a 50-mL round bottom flask, to a solution of dimethyl(prop-2-yn-1-yl)amine (1 g, 12 mmol) in THF (30 mL) was added dropwise n-butyllithium (2.5 mol/L, 5.8 mL, 14.4 mmol) at -78° C. under N₂ atmosphere. The reaction mixture was stirred at −78° C. for 30 min. Then a solution of tert-butyl N-(2-oxoethyl)carbamate (2.3 g, 14.4 mmol) in THF (10 mL) was added dropwise and the mixture was stirred for another 120 min. The reaction was quenched with water/sat. NH₄Cl (10 mL), and then the mixture was extracted with ether/EtOAc (2×50 mL). The combined organic extracts were washed with brine (30 mL), dried over anhydrous Na₂SO₄, concentrated under vacuum to yield a crude product which was directly purified by flash chromatography (DCM) to yield, tert-butyl N-[5-(dimethylamino)-2-hydroxypent-3-yn-1-yl]carbamate (130 mg, 4%) as a light yellow oil. LCMS (ESI) m/z: 243 [M+H]⁺.

Step 2: A solution of tert-butyl N-[5-(dimethylamino)-2-hydroxypent-3-yn-1-yl]carbamate (130 mg, 0.54 mmol) in HCl/1,4-dioxane (10 mL) was stirred for 2 h at room temperature. The resulting mixture was concentrated under reduced pressure. This resulted in 1-amino-5-(dimethylamino)pent-3-yn-2-ol (60 mg, 79%) as a light yellow oil, which was used in the next step directly without further purification. LCMS (ESI) m/z: 143 [M+H]⁺.

Intermediate 5 was used for the synthesis of Examples 31, 55, and 56.

### Intermediate 6: 1-amino-4-phenylbut-3-yn-2-ol

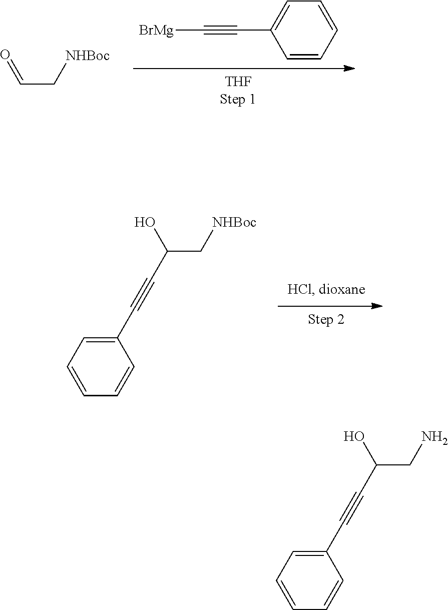

Step 1: To a stirred mixture of tert-butyl N-(2-oxoethyl)carbamate (1 g, 6.28 mmol) in THF (80 mL) was added bromo(2-phenylethynyl)magnesium (1M in THF, 20.1 mL, 20.1 mmol) dropwise at −78° C. under nitrogen atmosphere. The resulting mixture was stirred overnight at 60° C. under nitrogen atmosphere. The reaction was quenched with NH₄Cl at room temperature. The resulting mixture was extracted with EtOAc (2×200 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (1:1) to afford tert-butyl N-(2-hydroxy-4-phenylbut-3-yn-1-yl)carbamate (500 mg, 30%) as a yellow solid. LCMS (ESI) m/z: 262 [M+H]⁺.

Step 2: A mixture of tert-butyl N-(2-hydroxy-4-phenylbut-3-yn-1-yl)carbamate (250 mg, 0.96 mmol) and HCl in 1,4-dioxane (20 mL) was stirred for 1 h at room temperature under nitrogen atmosphere. The resulting mixture was concentrated under reduced pressure. The crude product mixture was used in the next step directly without further purification. LCMS (ESI) m/z: 162 [M+H]⁺.

Intermediate 6 was used for the synthesis of Example 36. In a similar manner using intermediates 1-aminopent-3-yn-2-ol and 1-amino-2-methylbut-3-yn-2-ol, Example 42, 271 and 272 respectively were synthesized.

### Intermediate 7: (3E)-1-amino-4-phenylbut-3-en-2-ol

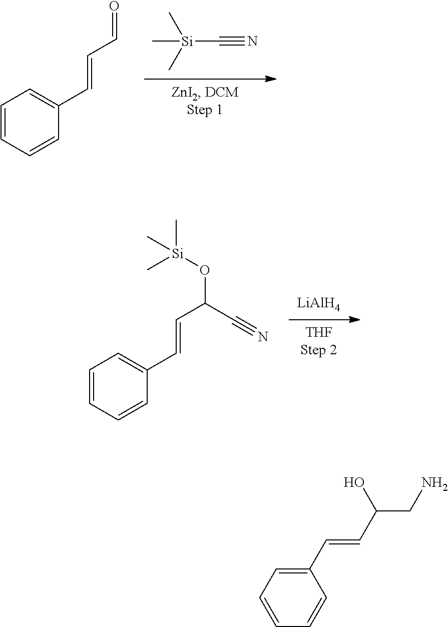

Step 1: A solution of cinnamal (1 g, 7.57 mmol) in DCM (15 mL) was treated with ZnI2 (0.24 g, 0.76 mmol) for 10 min at 0° C. under nitrogen atmosphere followed by the addition of trimethylsilyl cyanide (0.90 g, 9.08 mmol) dropwise at 0° C. The resulting mixture was stirred for 2 h at room temperature under nitrogen atmosphere. The resulting mixture was concentrated under reduced pressure to obtain (3E)-4-phenyl-2-[(trimethylsilyl)oxy]but-3-enenitrile (1.2 g, 68%) as a light yellow oil, which was used in the next step directly without further purification. LCMS (ESI) m/z: 232 [M+H]⁺.

Step 2: A solution of (3E)-4-phenyl-2-[(trimethylsilyl)oxy]but-3-enenitrile (1.2 g, 5.19 mmol) in lithium aluminum hydride (1.0 Min THF) (20 mL) was stirred for 2 h at room temperature. The reaction was quenched with water at room temperature. The resulting mixture was concentrated under reduced pressure to obtain (3E)-1-amino-4-phenylbut-3-en-2-ol (800 mg, 94%) as a light yellow oil, which was used in the next step directly without further purification. LCMS (ESI) m/z: 164 [M+H]⁺.

Intermediate 7 was used for the synthesis of Example 40. In a similar manner using appropriate starting material following intermediates were synthesized.

TABLE 29

| Example | |
|---|---|
| No. | Intermediate |
| 44 | 2-amino-1-(oxan-4-yl)ethanol |
| 45 | 2-amino-1-(4,4-difluorocyclohexyl)ethanol |
| 48 | 2-amino-1-cyclopentylethanol |
| 70 | 2-amino-1-(1-methylpiperidin-4-yl)ethan-1-ol |

### Intermediate 8: 1-amino-4-(1-methylcyclopropyl)but-3-yn-2-ol

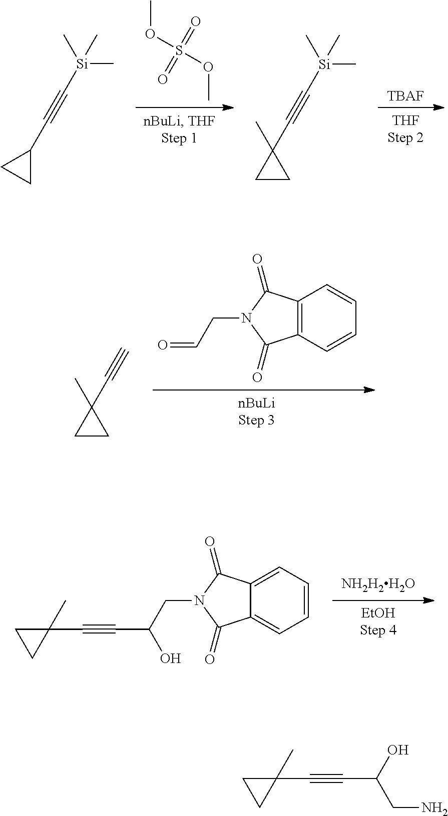

Step 1: A solution of (2-cyclopropylethynyl)trimethylsilane (6 g, 43.4 mmol) in THF (100 mL) was treated with n-BuLi (4169.2 mg, 65 mmol) for 30 min at −78° C. under nitrogen atmosphere followed by the addition of dimethyl sulfate (6.6 g, 52.1 mmol) dropwise at -78° C. The resulting mixture was stirred overnight at room temperature under nitrogen atmosphere. The reaction was quenched with water/sat. NH₄Cl (10 mL), and then the mixture was extracted with EtOAc (300 mL), dried over anhydrous Na₂SO₄. The product was precipitated by the addition of ethyl ether. This resulted in trimethyl[2-(1-methylcyclopropyl)ethynyl]silane (4 g) as a brown solid. LCMS (ESI) m/z: 153 [M+H]⁺.

Step 2: A solution of trimethyl[2-(1-methylcyclopropyl)ethynyl]silane (4 g, 26.26 mmol) and TBAF (8.24 g, 31.51 mmol) in THF (100 mL) was stirred for 2 h at room temperature under nitrogen atmosphere. The crude product was purified by distillation under atm and the fraction was collected at 80° C. The resulting mixture was concentrated under reduced pressure. This resulted in 1-ethynyl-1-methylcyclopropane (2 g) as a yellow oil. LCMS (ESI) m/z: 81 [M+H]⁺.

Step 3: A solution of 1-ethynyl-1-methylcyclopropane (2 g, 24.95 mmol) in THF (50 mL) was treated with phthalimide, N-(formylmethyl)-(5665.9 mg, 29.9 mmol) for 30 min at −78° C. under nitrogen atmosphere followed by the addition of butyllithium (2398.3 mg, 37.4 mmol) dropwise at −78° C. The resulting mixture was stirred overnight at room temperature under nitrogen atmosphere. The reaction was quenched with water/sat. NH₄Cl (10 mL), and then the mixture was extracted with EtOAc (300 mL), dried over anhydrous Na₂SO₄, the product was precipitated by the addition of ethyl ether. This resulted in 2-[2-hydroxy-4-(1-methylcyclopropyl)but-3-yn-1-yl]isoindole-1,3-dione (1 g) as a brown oil. LCMS (ESI) m/z: 270 [M+H]⁺.

Step 4: To a stirred solution of 2-[2-hydroxy-4-(1-methylcyclopropyl)but-3-yn-1-yl]isoindole-1,3-dione (1 g, 3.71 mmol) in anhydrous EtOH (20 mL) was added hydrazine hydrate (0.36 g, 7.42 mmol) at 70° C. and stirred for 4 h. The reaction progress was monitored by TLC. The resulting mixture was concentrated under reduced pressure. The crude product was used in the next step directly without further purification. This resulted in 1-amino-4-(1-methylcyclopropyl)but-3-yn-2-ol (300 mg) as a brown oil. LCMS (ESI) m/z: 140 [M+H]⁺.

Intermediate 8 was used for the synthesis of Example 49.

### Intermediate 9: Amino-5-[(tert-butyldimethylsilyl)oxy]-5-methylhex-3-yn-2-ol

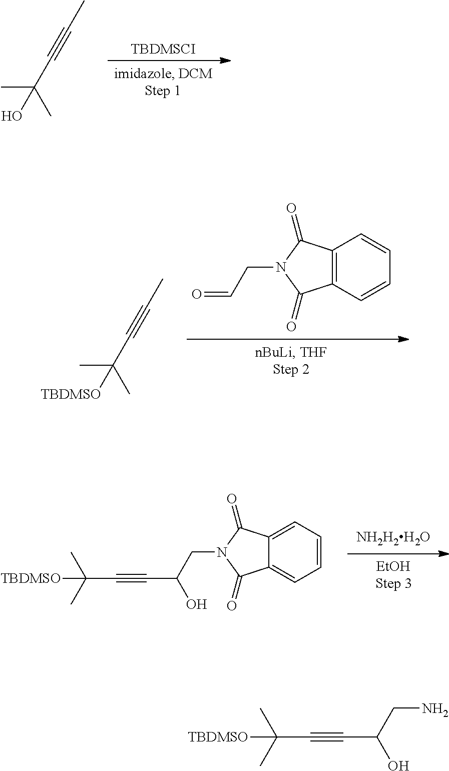

Step 1: A solution of 2-methyl-3-butyn-2-ol (3 g, 35.7 mmol) in DCM (50 mL) was treated with Imidazole (4.86 g, 71.3 mmol) for 1 min at room temperature under nitrogen atmosphere followed by the addition of t-butyldimethylchlorosilane (6.45 g, 42.8 mmol) at room temperature. The reaction mixture was stirred at room temperature for 16 h. The resulting mixture was extracted with EtOAc (3×50 mL). The combined organic layers were washed with brine (80 mL), dried over anhydrous MgSO₄. After filtration, the filtrate was concentrated under reduced pressure. The residue was purified by reversed-phase flash chromatography with the following conditions: column, C18 silica gel; mobile phase, MeCN in water (0.10% FA), 10% to 50% gradient in 10 min. This resulted in tert-butyldimethyl[(2-methylbut-3-yn-2-yl)oxy]silane (1.5 g) as a brown oil. LCMS (ESI) m/z: 199 [M+H]⁺.

Step 2: To a solution of tert-butyldimethyl[(2-methylbut-3-yn-2-yl)oxy]silane (1 g, 5.04 mmol) in THF (50 mL) was added dropwise n-butyllithium solution (2.5 M in THF, 5.04 mL, 12.6 mmol) at −78° C. under N₂ atmosphere. The reaction mixture was stirred at -78° C. for 30 mins. Then a solution of phthalimide, N-(formylmethyl)-(0.95 g, 5.04 mmol) in THF (10 mL) was added dropwise and the mixture was stirred for another 90 mins.

The reaction was quenched with water/sat. NH₄Cl (10 mL), and then the mixture was extracted with EtOAc (200 mL). The combined organic extracts were washed with brine (100 mL), dried over anhydrous Na₂SO₄. After filtration, the filtrate was concentrated under reduced pressure. The residue was purified by silica gel column chromatography, eluted with CH₂Cl₂/MeOH (8:1) to afford N-{5-[(tert-butyldimethylsilyl)oxy]-2-hydroxy-5-methylhex-3-yn-1-yl}-2-formylbenzamide (300 mg) as a brown oil.

Step 3: A solution of N-{5-[(tert-butyldimethylsilyl)oxy]-2-hydroxy-5-methylhex-3-yn-1-yl}-2-formylbenzamide (300 mg, 0.77 mmol) in EtOH (20 mL) was treated with NH₂NH₂·H₂O (57.8 mg, 1.15 mmol) for 1 min at room temperature. The reaction mixture was stirred at 70° C. for 2 h. The mixture was allowed to cool down to room temperature. The resulting mixture was filtered; the filter cake was washed with ethyl acetate (2×3 mL). The filtrate was concentrated under reduced pressure. This resulted in 1-amino-5-[(tert-butyldimethylsilyl)oxy]-5-methylhex-3-yn-2-ol (150 mg) as a brown oil, which was used in the next step directly without further purification. LCMS (ESI) m/z: 258 [M+H]⁺.

Intermediate 9 was used for the synthesis of Example 69.

### Intermediate 10: 1-amino-5-(diethylamino)pent-3-yn-2-ol

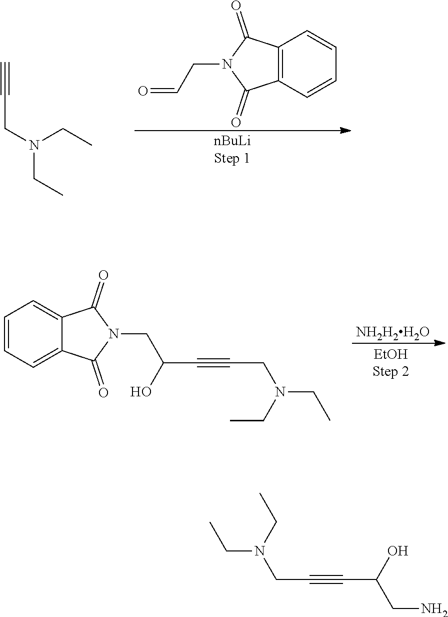

Step 1: To a solution of 2-propyn-1-amine, N, N-diethyl-(1 g, 9.0 mmol) in THF (10 mL) was added dropwise n-BuLi (3.9 mL, 9.89 mmol) at −78° C. under N₂ atmosphere. The reaction mixture was stirred at −78° C. for 30 min. Then a solution of phthalimide, N-(formylmethyl)-(1.7 g, 9 mmol) in THF (10 mL) was added dropwise and the mixture was stirred for another 1.5 h. The reaction was quenched with water/sat. NH₄Cl (10 mL), and then the mixture was extracted with EtOAc (2×15 mL). The combined organic extracts were washed with brine (10 mL), dried over anhydrous Na₂SO₄.The residue was purified by silica gel column chromatography, eluted with CH₂Cl₂/MeOH (12:1) to afford 2-[5-(diethylamino)-2-hydroxypent-3-yn-1-yl]isoindole-1,3-dione (500 mg, 18%) as a yellow oil. LCMS (ESI) m/z: 301 [M+H]⁺.

Step 2: A solution of 2-[5-(diethylamino)-2-hydroxypent-3-yn-1-yl]isoindole-1,3-dione (500 mg, 1.66 mmol) and NH₂NH₂·H₂O (166.7 mg, 3.33 mmol) in EtOH (10 mL) was stirred for 2 h at 80° C..The mixture was allowed to cool down to room temperature. The resulting mixture was filtered, the filter cake was washed with ethyl acetate (2×10 mL). The filtrate was concentrated under reduced pressure. This resulted in 1-amino-5-(diethylamino)pent-3-yn-2-ol (200 mg, 70%) as a yellow oil. LCMS (ESI) m/z: 171 [M+H]⁺.

Intermediate 10 was used for the synthesis of Example 81.

In a similar manner using appropriate starting material 1-amino-5-(pyrrolidin-1-yl)pent-3-yn-2-ol was synthesized. This was used for the synthesis of Example 87.

### Intermediate 11: 1-amino-5-[(tert-butyldimethylsilyl)oxy]pent-3-yn-2-ol

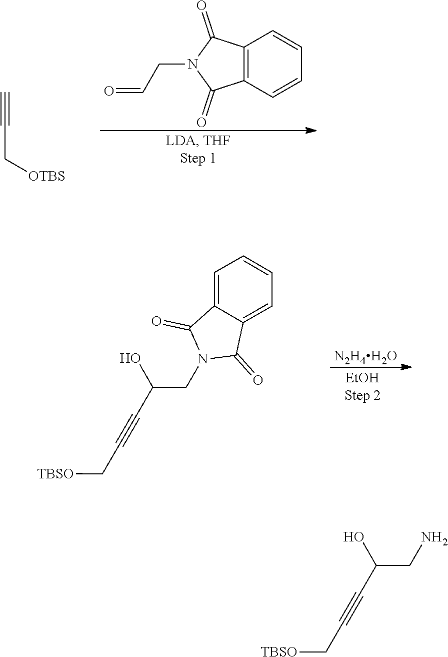 [SMILES: C#CCO[Si](C)(C)C(C)(C)C ; O=CCN1C(=O)c2ccccc2C1=O ; CC(C)(C)[Si](C)(C)OCC#CC(O)CN1C(=O)c2ccccc2C1=O ; "N2H4·H2O" ; CC(C)(C)[Si](C)(C)OCC#CC(O)CN]

Step 1: A solution of tert-butyldimethyl(prop-2-yn-1-yloxy)silane (5.4 g, 31.7 mmol) in THF (80 mL) was treated with LDA (2M in THF, 8.7 mL, 17.4 mmol) for 30 min at −78° C. under nitrogen atmosphere followed by the addition of phthalimide, N-(formylmethyl)-(3 g, 15.9 mmol) in portions at −78° C. The resulting mixture was stirred for 1 h at -50° C. under nitrogen atmosphere. The reaction was quenched by the addition of sat. NH₄Cl (aq.) (600 mL) at 0° C. The resulting mixture was extracted with EtOAc (3×500 mL). The combined organic layers were washed with brine (2×700 mL), dried over anhydrous Na₂SO₄.

After filtration, the filtrate was concentrated under reduced pressure. The residue was purified by silica gel column chromatography, eluted with PE/EA (20%-40%) to afford 2-{5-[(tert-butyldimethylsilyl)oxy]-2-hydroxypent-3-yn-1-yl}isoindole-1,3-dione (1.5 g, 26%) as a yellow oil. LCMS (ESI) m/z: 360 [M+H]⁺.

Step 2: A solution of 2-(5-((tert-butyldimethylsilyl)oxy)-2-hydroxypent-3-yn-1-yl)isoindoline-1,3-dione (1.5 g, 4.2 mmol) and hydrazine monohydrate (0.42 g, 8.3 mmol) in EtOH (15 mL) was stirred at 80° C. for 2 h. The resulting mixture was filtered, the filter cake was washed with Et₂O (3×30 mL). The filtrate was concentrated under reduced pressure to afford 1-amino-5-[(tert-butyldimethylsilyl)oxy]pent-3-yn-2-ol (800 mg, 83%) as a yellow oil. LCMS (ESI) m/z: 230 [M+H]⁺.

Intermediate 11 was used for the synthesis of Examples 99, 102 and 103.

### Intermediate 12: 2-amino-1-(3-methyl-1,2-oxazol-5-yl)ethanol

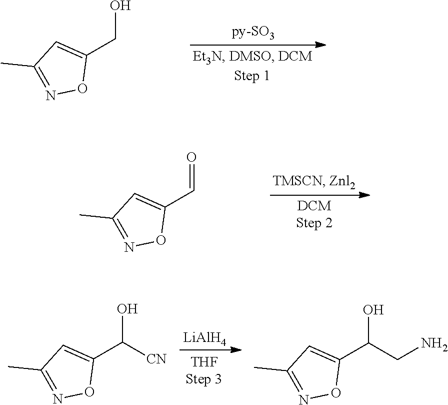

Step 1: To a stirred solution of (3-methyl-1,2-oxazol-5-yl)methanol (1 g, 8.84 mmol) and SO₃-pyridine (2.9 g, 18.6 mmol) in DCM (20 mL) were added Et₃N (4.5 g, 44.2 mmol) and DMSO (5.3 mL) at 0° C. under nitrogen atmosphere. The resulting mixture was stirred at 0° C. for 2 h under nitrogen atmosphere. The reaction was quenched by the addition of water (300 mL) at 0° C. The mixture was acidified to pH 4 with HCl (aq.). The resulting mixture was extracted with EtOAc (3×100 mL). The combined organic layers were washed with brine (2×100 mL), dried over anhydrous Na₂SO₄. After filtration, the filtrate was concentrated under reduced pressure to afford 3-methyl-1,2-oxazole-5-carbaldehyde (500 mg, 51%) as a yellow oil. LCMS (ESI) m/z: 112 [M+H]⁺.

Step 2: A mixture of 3-methyl-1,2-oxazole-5-carbaldehyde (500 mg, 4.5 mmol) and ZnI₂ (143.6 mg, 0.45 mmol) in DCM (20 mL) was stirred for 10 min at 0° C. under nitrogen atmosphere. To the above mixture was added trimethylsilyl cyanide (892.9 mg, 9 mmol) dropwise over 2 min at 0° C. The resulting mixture was stirred for additional 16 h at room temperature. The reaction was quenched by the addition of water (300 mL) at room temperature. The resulting mixture was extracted with CH₂Cl₂ (3×100 mL). The combined organic layers were washed with brine (2×100 mL), dried over anhydrous Na₂SO₄. After filtration, the filtrate was concentrated under reduced pressure to afford 2-hydroxy-2-(3-methyl-1,2-oxazol-5-yl)acetonitrile (500 mg, 80%) as a yellow solid. LCMS (ESI) m/z: 139 [M+H]⁺.

Step 3: To a stirred solution of 2-hydroxy-2-(3-methyl-1,2-oxazol-5-yl)acetonitrile (500 mg, 3.6 mmol) in THF (30 mL) was added LiAlH₄ (274.7 mg, 7.24 mmol) at 0° C. The resulting mixture was stirred for 1 h at 0° C. The reaction was quenched by the addition of Na₂SO₄.10H₂O (3 g) at 0° C. The resulting mixture was filtered, the filter cake was washed with THF (3×50 mL). The filtrate was concentrated under reduced pressure to afford 2-amino-1-(3-methyl-1,2-oxazol-5-yl)ethanol (400 mg, 78%) as a yellow solid. LCMS (ESI) m/z: 143 [M+H]⁺.

Intermediate 12 was used for the synthesis of Example 107.

### Intermediate 13: Amino-3-[(1-methylpyrazol-4-yl)oxy]propan-2-ol

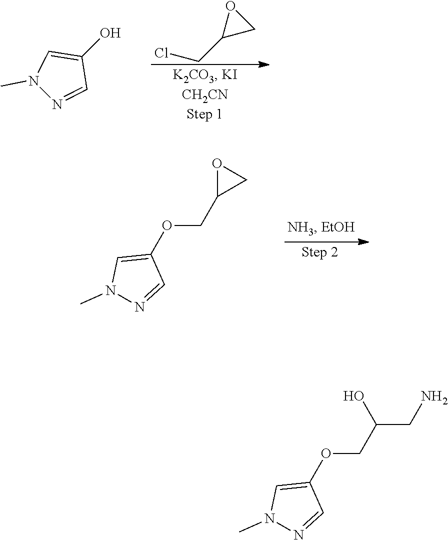

Step 1: To a stirred solution of 1-methylpyrazol-4-ol (2 g, 20.4 mmol) and epichlorohydrin (2.8 g, 30.6 mmol) in CH₃CN (30 mL) were added K₂CO₃ (8.4 g, 61.1 mmol) and KI (5.1 g, 30.6 mmol) at room temperature. The resulting mixture was stirred for 16 h at 80° C. The reaction was quenched by the addition of water (300 mL) at room temperature. The resulting mixture was extracted with EtOAc (3×100 mL). The combined organic layers were washed with brine (2×1 00 mL), dried over anhydrous Na₂SO₄. After filtration, the filtrate was concentrated underreduced pressure to afford 1-methyl-4-(oxiran-2-ylmethoxy)pyrazole (1.5 g, 48%) as a yellow oil. LCMS (ESI) m/z: 155.0 [M+H]⁺.

Step 2: A solution of 1-methyl-4-(oxiran-2-ylmethoxy)pyrazole (1.4 g, 9.4 mmol) in NH₃-EtOH (10 mL) was stirred for 2 h at 60° C. The resulting oil was dried under vacuum. The residue was purified by reversed-phase flash chromatography with the following conditions: column, C18 silica gel; mobile phase, MeCN in water (0.1% FA), 1% to 10% gradient in 20 min. The resulting mixture was dried under vacuum to afford 1-amino-3-[(1-methylpyrazol-4-yl)oxy]propan-2-ol (600 mg, 37%) as a yellow oil. LCMS (ESI) m/z: 172.0 [M+H]⁺. Intermediate 13 was used for the synthesis of Example 108. In a similar manner using appropriate starting material 1-amino-3-[(1-methylpyrazol-3-yl)oxy]propan-2-ol was synthesized without the use of KI and using DMF as the solvent. This intermediate was us ed to synthesize Examples 141.

Intermediate 14: tert-butyl N-[1-(4-amino-3-hydroxybut-1-yn-1-yl)cyclopropyl]-N-methylcarbamate

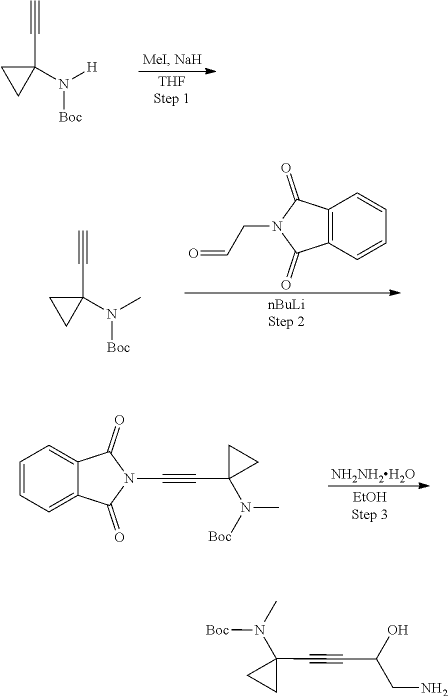 [SMILES: C#CC1(NC(=O)OC(C)(C)C)CC1 ; C#CC1(N(C)C(=O)OC(C)(C)C)CC1 ; O=CCN1C(=O)c2ccccc2C1=O ; CN(C(=O)OC(C)(C)C)C1(C#CN2C(=O)c3ccccc3C2=O)CC1 ; "NH2NH2·H2O" ; CN(C(=O)OC(C)(C)C)C1(C#CC(O)CN)CC1]

Step 1: To a solution of tert-butyl N-(1-ethynylcyclopropyl)carbamate (5 g, 27.6 mmol) in THF was added sodium hydride (60% in oil, 132.4 mg) at 0° C. The mixture was stirred for 15 min. Mel (4.7 g, 33.1 mmol) was added and the mixture was allowed to warm to room temperature and stirred at room temperature overnight. The reaction progress was monitored by TLC. The reaction was quenched by the addition of water/ice (5 mL) at 0° C. After completion of reaction, the reaction mixture was concentrated under reduced pressure to give crude product which was further purified by column chromatography (using 5% to 55% PE in DCM) to afford desired compound tert-butyl N-(1-ethynylcyclopropyl)-N-methylcarbamate (4 g) as a yellow liquid. Desired product was detected by TLC (PE/DCM=1:1, Rf=0.5). LCMS (ESI) m/z: 196 [M+H]⁺.

Step 2: To a solution of tert-butyl N-(1-ethynylcyclopropyl)-N-methylcarbamate (1 g, 5.12 mmol) in THF (50 mL) was added dropwise n-butyllithium solution (2.5 M in THF, 2.5 mL, 6.14 mmol) at −78° C. under N₂ atmosphere. The reaction mixture was stirred at -78° C. for 30 mins. Then a solution of 2-(1-oxo-3H-isoindol-2-yl)acetaldehyde (1.08 g, 6.14 mmol) in THF (20 mL) was added dropwise and the mixture was stirred for another 16 h. The reaction was quenched by the addition of water (15 mL) at room temperature. The resulting mixture was concentrated under reduced pressure. The residue was purified by silica gel column chromatography, eluted with CH₂Cl₂/MeOH (9:1) to afford tert-butyl (1-(4-(1,3-dioxoisoindolin-2-yl)-3-hydroxybut-1-yn-1-yl)cyclopropyl)(methyl)carbamate (1.2 g) as a brown oil. LCMS (ESI) m/z: 385 [M+H]⁺.

Step 3: A solution of tert-butyl (1-(4-(1,3-dioxoisoindolin-2-yl)-3-hydroxybut-1-yn-1-yl)cyclopropyl)(methyl)carbamate (1.2 g, 3.12 mmol) in EtOH (20 mL) was treated with NH₂NH₂·H₂O (0.31 g, 6.24 mmol) for 1 min at room temperature. The reaction mixture was stirred at 70° C. for 2 h. The mixture was allowed to cool down to room temperature. The resulting mixture was filtered, the filter cake was washed with ethyl acetate (3×20 mL), dried over anhydrous MgSO₄. The filtrate was concentrated under reduced pressure. This resulted in tert-butyl N-[1-(4-amino-3-hydroxybut-1-yn-1-yl)cyclopropyl]-N-methylcarbamate (400 mg) as a brown oil, which was used in the next step directly without further purification. LCMS (ESI) m/z: 255 [M+H]⁺.

Intermediate 14 was used for the synthesis of Examples 112, 127 and 129.

In a similar manner using EtI, tert-butylN-[1-(4-amino-3-hydroxybut-1-yn-1-yl)cyclopropyl]-N-ethylcarbamate was synthesized. This intermediate was used for the synthesis of Example 241.

### Intermediate 15: 1-Amino-3-(methylsulfanyl)propan-2-ol hydrochloride

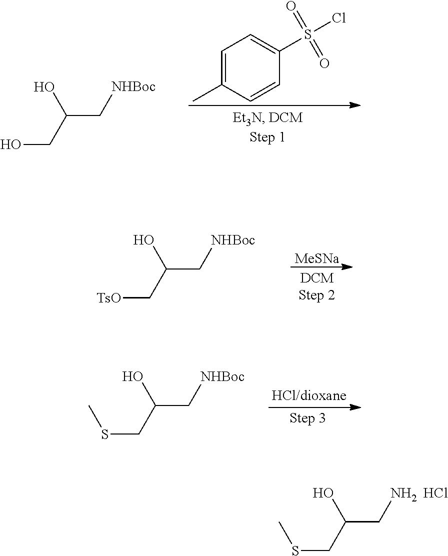

Step 1: To a stirred mixture of tert-butyl N-(2,3-dihydroxypropyl)carbamate (3 g, 15.7 mmol) and p-toluenesulfonyl chloride (3.6 g, 18.8 mmol) in DCM (40 mL) were added Et₃N (4.8 g, 47 mmol) dropwise at room temperature under nitrogen atmosphere. The resulting mixture was stirred at room temperature for 12 h. The reaction progress was monitored by TLC. Desired product was detected by TLC (PE/DCM=1:1, Rf=0.5). The resulting mixture was concentrated under reduced pressure. The residue was dissolved in H₂O (100 mL) and extracted with EtOAc (2×100 mL). The combined organic layers were washed with brine (50 mL), dried over anhydrous Na₂SO₄. After filtration, the filtrate was concentrated under reduced pressure to obtain product (3g). LCMS (ESI) m/z: 346 [M+H]⁺.

Step 2: A mixture of 3-((tert-butoxycarbonyl)amino)-2-hydroxypropyl 4-methylbenzenesulfonate (3 g, 8.68 mmol) and (methylsulfanyl)sodium (0.73 g, 10.4 mmol) in DCM (20 mL) was stirred at room temperature for 2 h. The resulting mixture was concentrated under reduced pressure. The residue was dissolved in H₂O (100 mL). The resulting mixture was extracted with EtOAc (2×100 mL). The combined organic layers were washed with brine (100 mL), dried over anhydrous Na₂SO₄. After filtration, the filtrate was concentrated under reduced pressure. The residue was purified by silica gel column chromatography, eluted with EA/PE (20-50%) to afford tert-butyl N-[2-hydroxy-3-(methylsulfanyl)propyl]carbamate (1 g, 52%) as a yellow oil. LCMS (ESI) m/z: 222 [M+H]⁺.

Step 3: A mixture of tert-butyl N-[2-hydroxy-3-(methylsulfanyl)propyl]carbamate (1 g, 4.51 mmol), HCl in 1,4-dioxane (10 mL) was stirred at room temperature for 2 h under nitrogen atmosphere. The resulting mixture was concentrated under reduced pressure to obtain 1-amino-3-(methylsulfanyl)propan-2-ol hydrochloride (500 mg, 70%) as a yellow oil, which was used in the next step directly without further purification. LCMS (ESI) m/z: 122 [M+H]⁺.

Intermediate 15 was used for the synthesis of Example 113.

### Intermediate 16: 1-Amino-3-cyclopropoxypropan-2-ol

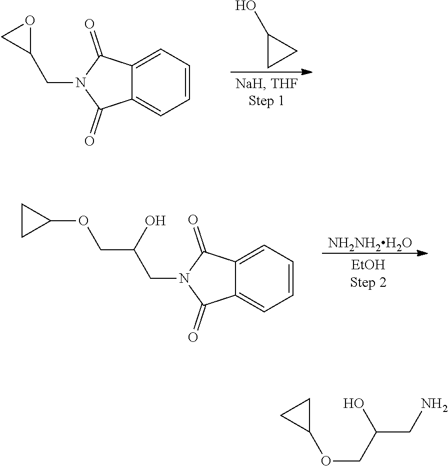

Step 1: To a solution of N-(2,3-epoxypropyl)phthalimide (2 g, 9.84 mmol) in THF (100 mL) was added sodium hydride (60% in oil, 0.47 g) at 0° C. The mixture was stirred for 15 min. Cyclopropanol (0.69 g, 11.8 mmol) was added and the mixture was allowed to warm to rt and stirred overnight. The reaction progress was monitored by TLC. Desired product was detected by TLC (PE/DCM=1:1, Rf=0.5). The reaction was quenched by the addition of water/ice (5 mL) at 0° C. After completion of reaction, the reaction mixture was concentrated under reduced pressure to give crude product which was further purified by column chromatography using PE in DCM (5% to 55%) gradient to afford desired compound 2-(3-cyclopropoxy-2-hydroxypropyl)isoindole-1,3-dione (1.4 g) as a brown oil.

Step 2: A solution of 2-(3-cyclopropoxy-2-hydroxypropyl)isoindole-1,3-dione (1.4 g, 5.35 mmol) in EtOH (20 mL) was treated with NH₂NH₂·H₂O (536.5 mg, 10.7 mmol) for 1 min at room temperature. The reaction mixture was stirred at 70° C. for 2 h. The mixture was allowed to cool down to room temperature. The resulting mixture was filtered, the filter cake was washed with ethyl acetate (3×20 mL), dried over anhydrous MgSO₄. The filtrate was concentrated under reduced pressure. This resulted in 1-amino-3-cyclopropoxypropan-2-ol (400 mg) as a brown oil which was used in the next step directly without further purification. LCMS (ESI) m/z: 132 [M+H]⁺.

Intermediate 16 was used for the synthesis of Example 115.

### Intermediate 17: 2-amino-1-cyclobutylethanol

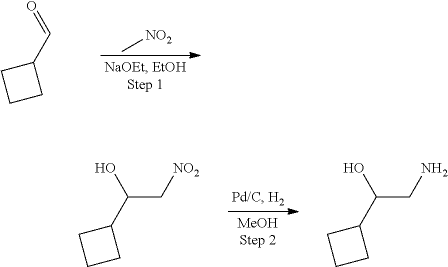

Step 1: A solution of cyclobutyral (1 g, 11.9 mmol) and EtONa (0.08 g, 1.19 mmol) in nitromethane (3 mL) and EtOH (9 mL) was stirred at room temperature for 16 h. The reaction was quenched by the addition of water (70 mL) at room temperature. The resulting mixture was extracted with EtOAc (2×80 mL). The combined organic layers were washed with brine (10 mL), dried over anhydrous Na₂SO₄. After filtration, the filtrate was concentrated under reduced pressure. This resulted in 1-cyclobutyl-2-nitroethanol (900 mg, 52%) as a yellow solid. LCMS (ESI) m/z: 146 [M+H]⁺.

Step 2: A solution of 1-cyclobutyl-2-nitroethanol (900 mg, 6.20 mmol) and Pd/C (501.5 mg, 4.71 mmol) in MeOH (30 mL) was stirred at room temperature for 16 h under hydrogen atmosphere. The resulting mixture was filtered, the filter cake was washed with MeOH (2×10 mL). The filtrate was concentrated under reduced pressure to obtain 2-amino-1-cyclobutylethanol (700 mg, 98%) as a clear oil. LCMS (ESI) m/z: 116 [M+H]⁺.

Intermediate 17 was used for the synthesis of Example 118. In a similar manner using appropriate starting material following intermediates were synthesized.

TABLE 30

| Example | |
|---|---|
| No. | Intermediate |
| 119 | 2-amino-1-(2-methyl-1,3-oxazol-4-yl)ethanol |
| 121 | 2-amino-1-(2-methyl-1,3-thiazol-5-yl)ethanol |
| 270 | tert-butyl N-[1-(2-amino-1- |
| | hydroxyethyl)cyclopropyl]carbamate |
| 276 | tert-butyl (2S)-2-(2-amino-1-hydroxyethyl)pyrrolidine-1- |
| | carboxylate |

Intermediate 18: tert-butyl N-[1-(4-amino-3-hydroxybut-1-yn-1-yl)cyclopropyl]-N-(tert-butoxycarbonyl)carbamate

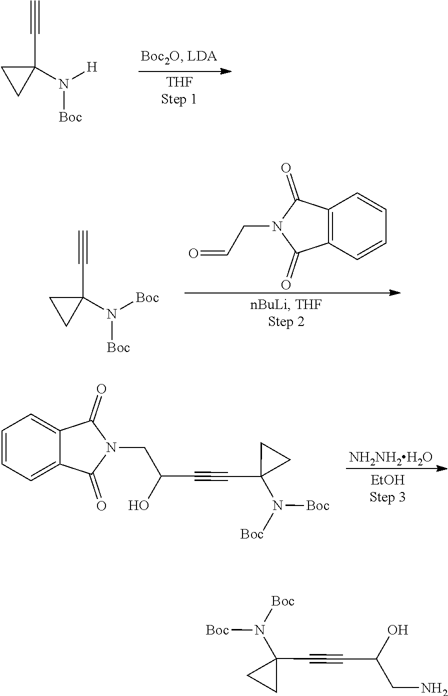

Step 1: A solution of tert-butyl N-(1-ethynylcyclopropyl)carbamate (3 g, 16.5 mmol) and LDA (2.0 M in THF/heptane) (2.13 g, 19.9 mmol) in THF (15 mL) was stirred at −78° C. for 1 h under nitrogen atmosphere. To the above mixture was added di-tert-butyl dicarbonate (3.97 g, 18.2 mmol) dropwise over 2 h at −78° C. The resulting mixture was stirred at −78° C. for additional 2 h. The reaction was quenched with sat. NH₄Cl (aq.) at room temperature. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (12:1) to afford tert-butyl N-(tert-butoxycarbonyl)-N-(1-ethynylcyclopropyl)carbamate (2.5 g, 54% yield, 90% purity) as a white oil. LCMS (ESI) m/z: 282 [M+H]⁺.

Step 2: A solution of tert-butyl N-(tert-butoxycarbonyl)-N-(1-ethynylcyclopropyl)carbamate (2.5 g, 8.89 mmol) in THF was treated with n-BuLi (2.5 Min n-hexane) (0.68 g, 10.7 mmol) at -78° C. for 30 min under nitrogen atmosphere followed by the addition of 2-(1,3-dioxoisoindolin-2-yl)acetaldehyde (1.68 g, 8.89 mmol) at −78° C. The resulting mixture was stirred at −78° C. for 1 h under nitrogen atmosphere. The reaction was quenched by the addition of water at room temperature and extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with CH₂Cl₂/MeOH (12:1) to afford tert-butyl N-(tert-butoxycarbonyl)-N-(1-[4-(1,3-dioxoisoindol-2-yl)-3-hydroxybut-1-yn-1-yl]cyclopropylcarbamate (1.5 g, 36% yield, 85% purity) as a yellow solid. LCMS (ESI) m/z: 471 [M+H]⁺.

Step 3: A solution of tert-butyl N-(tert-butoxycarbonyl)-N-(1-[4-(1,3-dioxoisoindol-2-yl)-3-hydroxybut-1-yn-1-yl]cyclopropylcarbamate (1.5 g, 3.19 mmol) and hydrazine monohydrate (319.2 mg, 6.38 mmol) in EtOH (20 mL) was stirred at 60° C. for 2 h. The resulting mixture was filtered, the filter cake was washed with EtOH (2×10 mL). The filtrate was concentrated under reduced pressure. The crude product was used in the next step directly without further purification. LCMS (ESI) m/z: 341 [M+H]⁺.

Intermediate 18 was used for the synthesis of Example 124.

### Intermediate 19: 1-amino-3-(pyridin-2-ylamino)propan-2-ol

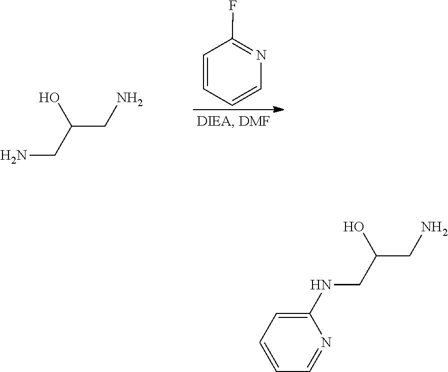 [SMILES: NCC(O)CN ; Fc1ccccn1 ; NCC(O)CNc1ccccn1]

To a stirred solution of 2-fluoropyridine (1 g, 10.3 mmol) and 1,3-diaminopropan-2-ol (1.11 g, 12.4 mmol) in DMF (10 mL) was added DIEA (3.99 g, 30.9 mmol) at room temperature. The resulting mixture was stirred at 100° C. for 16 h. The resulting mixture was dried under vacuum. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm to afford 1-amino-3-(pyridin-2-ylamino)propan-2-ol (500 mg, 29%) as a yellow oil. LCMS (ESI) m/z: 168 [M+H]⁺.

Intermediate 19 was used for the synthesis of Example 131.

### Intermediate 20: p-[(R)-3-amino-2-hydroxypropoxy]benzonitrile

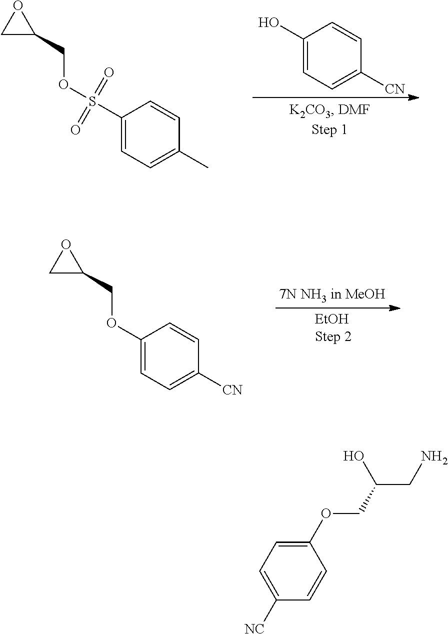

Step 1: To a solution of p-hydroxybenzonitrile (100 mg, 0.84 mmol) in DMF (2.6 mL) added (R)-2-[(tosyloxy)methyl]oxirane (287 mg, 1.26 mmol), K₂CO₃ (232 mg, 1.68 mmol) and heated at 65° C. overnight. The reaction was cooled, quenched with water, and extracted with EtOAc. The organic phases were combined, washed with water and brine, and dried over sodium sulfate. The solvent was removed in vacuo to afford a colorless residue that was used in the subsequent step without further purification.

Step 2: The residue from previous step was dissolved in MeOH (2.8 mL) and added 7M NH₃/MeOH (1.2 mL, 8.4 mmol). The reaction was stirred for 1 hour at room temperature. The solvent was removed in vacuo to afford the desired product as a colorless oil that was used in the subsequent step without further purification (144 mg, 89%). LCMS (ESI) m/z: 193 [M+H]⁺.

Intermediate 20 was used for the synthesis of Example 135. In a similar manner using appropriate starting material following intermediates were synthesized.

TABLE 31

| Example | |
|---|---|
| No. | Intermediate |
| 136 | m-[(R)-3-amino-2-hydroxypropoxy]benzonitrile |
| 137 | o-[(R)-3-amino-2-hydroxypropoxy]benzonitrile |
| 138 | (R)-1-amino-3-(m-chlorophenoxy)-2-propanol |
| 143 | (R)-1-amino-3-(p-chlorophenoxy)-2-propanol |
| 150 | (R)-3-amino-1-(3-isoxazolyloxy)-2-propanol |
| 151 | (R)-1-amino-3-(o-fluorophenoxy)-2-propanol |

### Intermediate 21: (1-fluorocyclopropyl) methanol

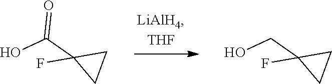 [SMILES: O=C(O)C1(F)CC1 ; OCC1(F)CC1]

A solution of 1-fluorocyclopropane-1-carboxylic acid (2 g, 19.2 mmol) in THF (50 mL) was treated with Lithium aluminum hydride (922.3 mg, 38.4 mmol) for 1 min at 0° C. under nitrogen atmosphere. The reaction mixture was stirred at room temperature for 2 h. The reaction was quenched with water at 0° C., dried over anhydrous Na₂SO₄. After filtration, the filtrate was concentrated under reduced pressure. The resulting mixture was concentrated under reduced pressure. This resulted in (1-fluorocyclopropyl) methanol (800 mg) as a white oil. The reaction progress was monitored by TLC. Desired product was detected by TLC (PE/DCM=1:1, Rf=0.5). LCMS (ESI) m/z: 91 [M+H]⁺.

Intermediate 21 was used for the synthesis of Example 152.

### Intermediate 22: (S)-2-amino-3-butyn-1-ol-hydrogen chloride (1/1)

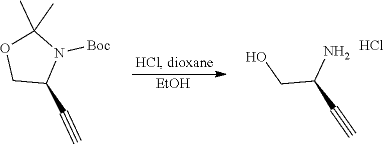

To a solution of tert-butyl (S)-4-ethynyl-2,2-dimethyl-1,3-oxazolidine-3-carboxylate (175 mg, 0.78 mmol) in ethanol (2.1 mL) added a 4M solution of hydrogen chloride in 1, 4-dioxane (1.5 mL). The reaction was stirred at 60° C. overnight. The mixture was cooled to room temperature and the solvent was removed in vacuo to afford a brown residue that was used directly in the next step.

Intermediate 22 was used for the synthesis of Example 156.

### Intermediate 23: (1-(prop-2-yn-1-yl)-1H-pyrazol-4-yl)methanol

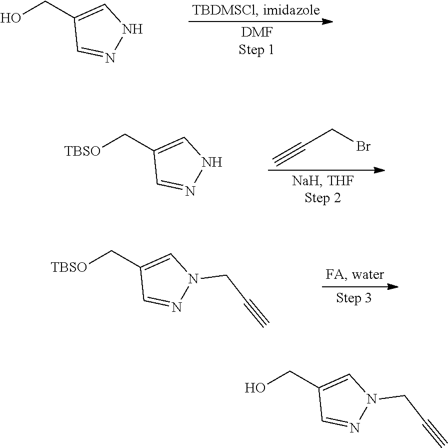

Step 1: To a stirred mixture of 1H-pyrazol-4-ylmethanol (3 g, 30.6 mmol) and imidazole (4.16 g 61.2 mmol) in DMF (30 mL) was added TBDMSCl (6.91 g, 45.9 mmol) in portions at room temperature. The resulting mixture was stirred at room temperature for 1 h. The reaction was quenched by the addition of water (400 mL) at room temperature. The resulting mixture was extracted with EtOAc (2×400 mL). The combined organic layers were washed with brine (400 mL), dried over anhydrous Na₂SO₄. After filtration, the filtrate was concentrated under reduced pressure. The residue was purified by silica gel column chromatography, eluted with MeOH/CH₂Cl₂ (0-25%) to afford 4-{[(tert-butyldimethylsilyl)oxy]methyl}-1H-pyrazole (2.4 g, 37%, 60% purity) as a yellow oil. LCMS (ESI) m/z: 213 [M+H]⁺.

Step 2: A solution of 4-{[(tert-butyldimethylsilyl)oxy]methyl}-1H-pyrazole (2.4 g, 11 mmol) in THF (20 mL) was treated with NaH (0.32 g, 13.3 mmol) at 0° C. for 30 min under nitrogen atmosphere followed by the addition of propargyl bromide (2.6 g, 22.1 mmol) dropwise at 0° C. The resulting mixture was stirred at room temperature for 16 h under nitrogen atmosphere. The resulting mixture was concentrated under reduced pressure. The reaction was quenched by the addition of water (50 mL) at room temperature and extracted with EtOAc (2×50 mL). The combined organic layers were washed with brine (50 mL), dried over anhydrous Na₂SO₄. After filtration, the filtrate was concentrated under reduced pressure. The residue was purified by silica gel column chromatography, eluted with MeOH/CH₂Cl₂ (0-20%) to afford 4-{[(tert-butyldimethylsilyl)oxy]methyl}-1-(prop-2-yn-1-yl)pyrazole (1 g, 36%) as a yellow oil. LCMS (ESI) m/z: 251 [M+H]⁺.

Step 3: A mixture of 4-{[(tert-butyldimethylsilyl)oxy]methyl}-1-(prop-2-yn-1-yl)pyrazole (0.9 g, 3.59 mmol) in H₂O (5 mL) and HCOOH (5 mL) was stirred at room temperature for 2 h. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 30 min; detector, UV 254 nm. This resulted in (1-(prop-2-yn-1-yl)-1H-pyrazol-4-yl)methanol (0.8 g) as a white oil. LCMS (ESI) m/z: 137 [M+H]⁺.

Intermediate 23 was used for the synthesis of Example 162.

### Intermediate 24: (4R)-4-amino-1-pentyn-3-ol

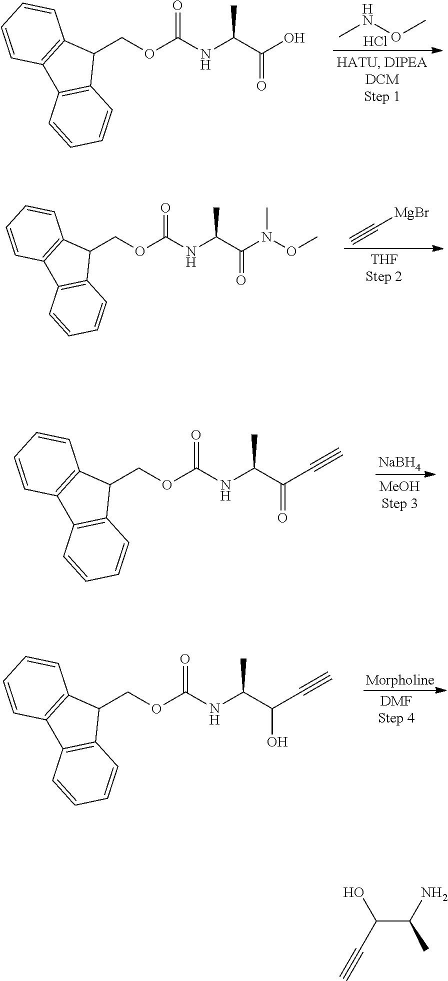

Step 1: To a solution of (S)-2-{[(9H-fluoren-9-yl)methyl](oxycarbonylamino)}propionic acid (2 g, 6.42 mmol) in DCM (21.4 mL) added O-methyl-N-methylhydroxylamine-hydrogen chloride (1/1) (689 mg, 7.07 mmol), HATU (3.7 g, 9.64 mmol) and DIPEA (3.4 mL, 19.3 mmol). The mixture was stirred at room temperature overnight. The solution was quenched with water, extracted with DCM. The organic phases were combined, dried over sodium sulfate and concentrated in vacuo. The residue was purified via flash chromatography (0-100% Hex: [25% EtOH in EtOAc), to afford (S)-1-(N-methoxy-N-methylcarbamoyl)ethyl (9H-fluoren-9-yl)methanecarbamate as a white solid (2 g, 86%). LCMS (ESI) m/z: 355 [M+H]⁺.

Step 2: To (S)-1-(N-methoxy-N-methylcarbamoyl)ethyl (9H-fluoren-9-yl)methanecarbamate (0.5 g, 1.41 mmol) in THF (10 mL) at 0° C. was added magnesium bromide ethynide (547 mg, 4.23 mmol). The solution was warmed to room temperature and stirred for 4 hours. The solution was quenched with 1 M HCl and stirred for 20 min and extracted with EtOAc. The organic phases were combined, dried over sodium sulfate, filtered and had solvent removed in vacuo. The residue was purified via flash chromatography (0-100% Hex: [25% EtOH in EtOAc), to afford (S)-1-methyl-2-oxo-3-butynyl (9H-fluoren-9-yl)methanecarbamate as a white solid (408 mg, 90%).

Step 3: (S)-1-methyl-2-oxo-3-butynyl (9H-fluoren-9-yl)methanecarbamate (451 mg, 1.41 mmol) was dissolved in MeOH (9 mL) and cooled to 0° C. Subsequently added NaBH₄ (58.8 mg, 1.55 mmol). The mixture was warmed to room temperature and stirred for 30 min. The mixture was quenched with NH₄Cl aq solution and extracted with EtOAc. The organic phases were combined, dried over sodium sulfate, filtered and solvent was removed in vacuo. The residue was purified by column chromatography (0-50% hex: EtOAc) to afford (1S)-2-hydroxy-1-methyl-3-butynyl (9H-fluoren-9-yl)methanecarbamate as a yellow oil (198 mg, 44%). LCMS (ESI) m/z: 322 [M+H]⁺.

Step 4: To (1S)-2-hydroxy-1-methyl-3-butynyl (9H-fluoren-9-yl)methanecarbamate (0.1 g, 0.31 mmol) in DMF (1 mL) added morpholine (271 mg, 3.1 mmol) and the reaction was stirred at room temperature for 2 h. The cloudy solution was filtered and the filtrate was dried in vacuo to afford (4R)-4-amino-1-pentyn-3-ol which was used in the next step without further purification.

Intermediate 24 was used for the synthesis of Example 173.

### Intermediate 25: tert-butyl 3-(hydroxymethyl)-4H,6H,7H-pyrazolo[1,5-a]pyrazine-5-carboxylate

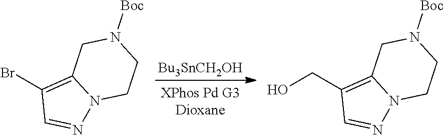 [SMILES: "Bu3SnCH2OH" ; CC(C)(C)OC(=O)N1CCn2ncc(Br)c2C1 ; CC(C)(C)OC(=O)N1CCn2ncc(CO)c2C1]

To a stirred mixture of tert-butyl 3-bromo-4H,6H,7H-pyrazolo[1,5-a]pyrazine-5-carboxylate (3 g, 9.93 mmol) and (tributylstannyl)methanol (6.4 g, 19.8 mmol) in dioxane (30 mL) were added XPhos Pd G3 (0.84 g, 0.99 mmol) in portions at room temperature under nitrogen atmosphere. The resulting mixture was stirred at 90° C. for 2 h under nitrogen atmosphere. The resulting mixture was concentrated under reduced pressure. The residue was purified by silica gel column chromatography, eluted with MeOH/CH₂Cl₂ (0-15%) to afford tert-butyl 3-(hydroxymethyl)-4H,6H,7H-pyrazolo[1,5-a]pyrazine-5-carboxylate (800 mg, 32%) as a brown oil. LCMS (ESI) m/z: 254 [M+H]⁺.

Intermediate 25 was used for the synthesis of Examples 177, 288, 290 and 297.

### Intermediate 26: ({3-bromoimidazo[1,2-b]pyridazin-7-yl}methyl)dimethylamine

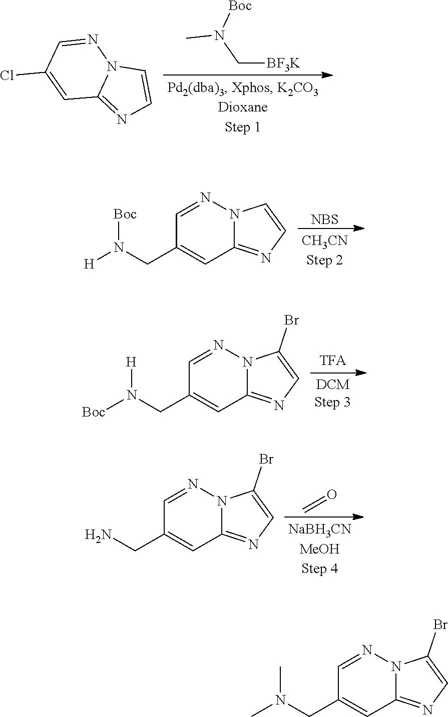

Step 1: To a stirred solution of 7-chloroimidazo[1,2-b]pyridazine (500 mg, 3.25 mmol) and potassium tert-butyl N-[(trifluoroboranuidyl)methyl]carbamate (1543.7 mg, 6.51 mmol) in 1,4-dioxane (20 mL), H₂O (2 mL) were added K₂CO₃ (1349.9 mg, 9.76 mmol), XPhos (310.4 mg, 0.65 mmol) and Pd₂(dba)₃ (73.1 mg, 0.32 mmol) at room temperature. The resulting mixture was stirred at 100° C. for 2 h under nitrogen atmosphere. The resulting mixture was dried under vacuum. The residue was purified by silica gel column chromatography, eluted with PE/EA (50%-70%) to afford tert-butyl N-{imidazo[1,2-b]pyridazin-7-ylmethyl}carbamate (700 mg, 87%, 72% purity) as a yellow solid. LCMS (ESI) m/z: 249 [M+H]⁺.

Step 2: To a stirred solution of tert-butyl N-{imidazo[1,2-b]pyridazin-7-ylmethyl}carbamate (700 mg, 2.81 mmol) in MeCN (10 mL) was added NBS (526.9 mg, 2.96 mmol) at 0° C. The resulting mixture was stirred at room temperature for 2 h. The resulting mixture was dried under vacuum. The residue was purified by silica gel column chromatography, eluted with PE/EA (70%-90%) to afford tert-butyl N-({3-bromoimidazo[1,2-b]pyridazin-7-yl}methyl)carbamate (800 mg, 87%, 83% purity) as a yellow solid. LCMS (ESI) m/z: 327, 329 [M+H]⁺.

Step 3: To a stirred solution of tert-butyl N-({3-bromoimidazo[1,2-b]pyridazin-7-yl}methyl)carbamate (300 mg, 0.91 mmol) in DCM (12 mL) was added trifluoroacetic acid (4 mL) at 0° C. The resulting mixture was stirred at room temperature for 1 h. The resulting oil was dried under vacuum to afford 1-{3-bromoimidazo[1,2-b]pyridazin-7-yl}methanamine (200 mg) as a brown oil. LCMS (ESI) m/z: 227, 229 [M+H]⁺.

Step 4: To a stirred solution of 1-{3-bromoimidazo[1,2-b]pyridazin-7-yl}methanamine (170 mg, 0.74 mmol) and formaldehyde (224.8 mg, 2.24 mmol, 30%) in methanol (10 mL) was added NaBH₃CN (94.1 mg, 1.49 mmol) at 0° C. The resulting mixture was stirred at room temperature for 16 h. The pH was adjusted to pH 9 with saturated Na₂CO₃ (aq.). The resulting mixture was extracted with EtOAc (3×80 mL). The combined organic layers were washed with brine (2×80 mL), dried over anhydrous Na₂SO₄. After filtration, the filtrate was concentrated under reduced pressure. The residue was purified by silica gel column chromatography, eluted with CH₂Cl₂/MeOH (0%-10%) to afford ({3-bromoimidazo[1,2-b]pyridazin-7-yl}methyl)dimethylamine (120 mg, 63%) as a yellow solid. LCMS (ESI) m/z: 255, 257 [M+H]⁺.

Intermediate 26 was used for the synthesis of Example 191.

### Intermediate 27: [(5-bromo-1-methylpyrazol-3-yl) methyl]dimethylamine

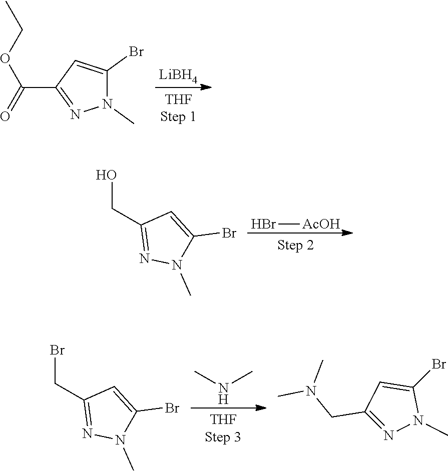

Step 1: To a solution of ethyl 5-bromo-1-methylpyrazole-3-carboxylate (2 g, 8.58 mmol) in THF (50 mL) under N₂ atmosphere added LiBH₄ (0.38 g, 17.2 mmol) in portions at 0° C. The resulting mixture was stirred at 0° C. for 1 h under nitrogen atmosphere. Desired product was detected by TLC (PE/DCM=1:1, Rf=0.5). The reaction was quenched with Na₂SO₄ 0.10 H₂O at 0° C. The resulting mixture was filtered. The filter cake was washed with THF (3×100 mL). The filtrate was concentrated under reduced pressure to afford (5-bromo-1-methylpyrazol-3-yl) methanol (1 g) as a colorless oil. LCMS (ESI) m/z: 191,193 [M+H]⁺.

Step 2: A solution of (5-bromo-1-methylpyrazol-3-yl) methanol (1 g, 5.23 mmol) in hydrogen bromide-acetic acid solution (10 mL) was stirred at 100° C. for 16 h under nitrogen atmosphere. Desired product was detected by TLC (PE/EA=1:1, Rf=0.5). The mixture was concentrated under vacuum. The residue was treated with Et₂O (20 mL). The precipitated solids were collected by filtration and washed with Et₂O (3×50 mL). The resulting solid was dried under reduced pressure to afford 5-bromo-3-(bromomethyl)-1-methylpyrazole (420 mg, crude) as a brown solid. LCMS (ESI) m/z: 253, 255 [M+H]⁺.

Step 3: A solution of 5-bromo-3-(bromomethyl)-1-methylpyrazole (420 mg, 1.65 mmol) in dimethylamine in THF (2.0 M in THF, 10 mL) was stirred at room temperature for 2 h under nitrogen atmosphere. The resulting mixture was concentrated under vacuum. The residue was washed with water and dried under vacuum to afford [(5-bromo-1-methylpyrazol-3-yl) methyl]dimethylamine (300 mg, crude) as a brown solid. LCMS (ESI) m/z: 218, 220 [M+H]

Intermediate 27 was used for the synthesis of Example 195.

### Intermediate 28: 3-bromo-N-methyl-5H,6H,7H-pyrazolo[3,2-b][1,3]oxazin-6-amine

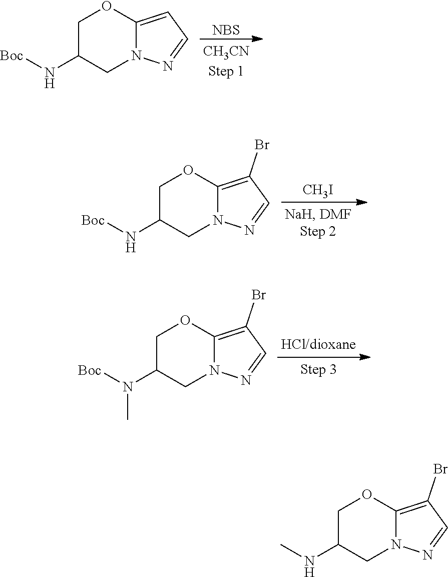

Step 1: To a stirred solution of tert-butyl N-{5H,6H,7H-pyrazolo[3,2-b][1,3]oxazin-6-yl}carbamate (1.5 g, 6.27 mmol) in MeCN (12 mL) was added NBS (1115.8 mg, 6.27 mmol) at 0° C. under nitrogen atmosphere. The resulting mixture was stirred at room temperature for 2 h under nitrogen atmosphere. The reaction was quenched by the addition of water (60 mL) at room temperature. The resulting mixture was extracted with EtOAc (2×100 mL). The combined organic layers were washed with brine (2×10 mL), dried over anhydrous Na₂SO₄. After filtration, the filtrate was concentrated under reduced pressure. This resulted in tert-butyl N-{3-bromo-5H,6H,7H-pyrazolo[3,2-b][1,3]oxazin-6-yl}carbamate (1.8 g, 90%) as a white solid. LCMS (ESI) m/z: 318, 320 [M+H]⁺.

Step 2: To a stirred solution of tert-butylN-{3-bromo-5H,6H,7H-pyrazolo[3,2-b][1,3]oxazin-6-yl}carbamate (1.8 g, 5.66 mmol) in DMF (30 mL) was added NaH (0.45 g, 11.3 mmol) in portions at 0° C. under nitrogen atmosphere. The resulting mixture was stirred at 0° C. for 1 h under nitrogen atmosphere. To the above mixture was added iodomethane (1.61 g, 11.3 mmol) at 0° C. The resulting mixture was stirred at room temperature for additional 2 h. The reaction was quenched by the addition of water (100 mL) at room temperature. The resulting mixture was extracted with EtOAc (2×100 mL). The combined organic layers were washed with brine (10 mL), dried over anhydrous Na₂SO₄. After filtration, the filtrate was concentrated under reduced pressure. The residue was purified by silica gel column chromatography, eluted with PE/EA (1:1) to afford tert-butyl N-{3-bromo-5H,6H,7H-pyrazolo[3,2-b][1,3]oxazin-6-yl}-N-methylcarbamate (1.5 g, 80%) as a white solid. LCMS (ESI) m/z: 332, 334 [M+H]⁺.

Step 3: A solution of tert-butyl N-{3-bromo-5H,6H,7H-pyrazolo[3,2-b][1,3]oxazin-6-yl}-N-methylcarbamate (800 mg, 2.41 mmol) in HCl in 1,4-dioxane (4.0 M) (15 mL) was stirred at room temperature for 1 h. The resulting mixture was concentrated under reduced pressure. The resulting mixture was washed with diethyl ether (2×10 mL). The precipitated solids were collected by filtration and washed with diethyl ether (2×5 mL). This resulted in 3-bromo-N-methyl-5H,6H,7H-pyrazolo[3,2-b][1,3]oxazin-6-amine (600 mg) as a white solid. LCMS (ESI) m/z: 232, 234 [M+H]⁺.

Intermediate 28 was used for the synthesis of Example 205.

### Intermediate 29: 3-bromoimidazo[1,2-b]pyridazine-7-carbonitrile

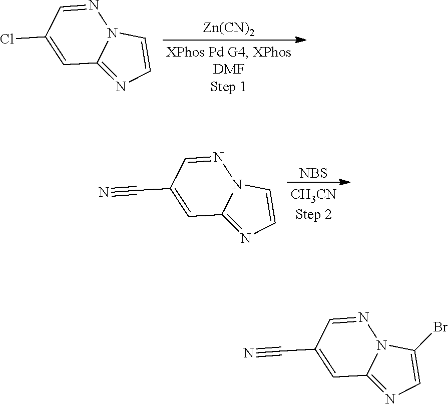

Step 1: To a stirred solution of 7-chloroimidazo[1,2-b]pyridazine (300 mg, 1.95 mmol) and zinc cyanide (183.5 mg, 1.56 mmol) in DMF (8 mL) were added XPhos Pd G4 (152.4 mg, 0.19 mmol) and XPhos (93.1 mg, 0.19 mmol) at room temperature. The resulting mixture was stirred at 110° C. for 3 h. The reaction was quenched by the addition of water (100 mL) at room temperature. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with EA/PE (30%-50%) to afford imidazo[1,2-b]pyridazine-7-carbonitrile (220 mg, 78%) as a yellow solid. LCMS (ESI) m/z: 145.0 [M+H]⁺.

Step 2: A solution of imidazo[1,2-b]pyridazine-7-carbonitrile (200 mg, 1.38 mmol) and NBS (259.3 mg, 1.45 mmol) in MeCN (10 mL) was stirred at room temperature for 1 h. Desired product could be detected by LCMS. The resulting mixture was dried under vacuum. The residue was purified by silica gel column chromatography, eluted with EA/PE (40%-60%) to afford 3-bromoimidazo[1,2-b]pyridazine-7-carbonitrile (260 mg, 84%) as a yellow solid. LCMS (ESI) m/z: 223.0, 225.0 [M+H]⁺.

Intermediate 29 was used for the synthesis of Example 206.

### Intermediate 30: 1-(difluoromethyl)-5-iodopyrazole

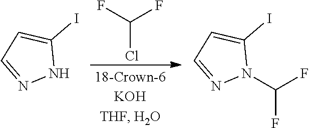

A mixture of 3-iodo-2H-pyrazole (3 g, 15.47 mmol), 18-crown-6 (0.41 g, 1.55 mmol) and KOH (2.6 g, 46.34 mmol) in THF (10 mL) and H₂O (1 mL) was stirred at 40° C. for 2 h under chlorodifluoromethane atmosphere. The reaction was quenched by the addition of water (20 mL) at room temperature. The resulting mixture was extracted with CH₂Cl₂ (2×20 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EA (12:1) to afford 1-(difluoromethyl)-5-iodopyrazole (300 mg) as a colorless oil. LCMS (ESI) m/z: 245 [M+H]⁺.

Intermediate 30 was used for the synthesis of Example 210.

### Intermediate 31: 5-iodo-1-(2H3)methylpyrazole

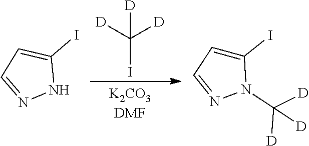

A solution of 3-iodo-2H-pyrazole (2 g, 10.31 mmol), iodo(2H3)methane (2.99 g, 20.6 mmol), K₂CO₃ (4.27 g, 30.9 mmol) in DMF (20 mL) was stirred at room temperature for 24 h. The residue was dissolved in H₂O (20 mL) and extracted with EtOAc (3×20 mL). The combined organic layers were washed with H₂O (3×20 mL), dried over anhydrous Na₂SO₄. After filtration, the filtrate was concentrated under reduced pressure. The residue was purified by reversed-phase flash chromatography with the following conditions: column, C18 silica gel; mobile phase, MeOH in Water (0.1% FA), 40% to 50% gradient in 10 min; detector, UV 254 nm. This resulted in 5-iodo-1-(2H3)methylpyrazole (500 mg) as a yellow solid. LCMS (ESI) m/z: 212 [M+H]⁺.

Intermediate 31 was used for the synthesis of Example 211.

### Intermediate 32: 5-bromo-4-fluoro-1-methylpyrazole

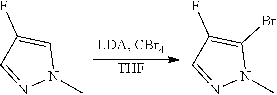

A solution of 4-fluoro-1-methylpyrazole (500 mg, 4.99 mmol) in THF (20 mL) was treated with LDA (2M in THF, 3.00 mL, 5.99 mmol) at −78° C. for 1 h under nitrogen atmosphere followed by the addition of tetrabromomethane (1987.8 mg, 5.99 mmol) at −78° C. The resulting mixture was stirred at −78° C. for 2 h under nitrogen atmosphere. The reaction was quenched by the addition of sat. NH₄Cl (aq.) (50 mL) at 0° C. The resulting mixture was extracted with EtOAc (2×50 mL). The combined organic layers were washed with brine (10 mL), dried over anhydrous Na₂SO₄. After filtration, the filtrate was concentrated under reduced pressure. The residue was purified by silica gel column chromatography, eluted with PE/EA (9:1) to afford 5-bromo-4-fluoro-1-methylpyrazole (280 mg, 31%) as a black liquid. LCMS (ESI) m/z: 179,181 [M+H]⁺.

Intermediate 32 was used for the synthesis of Example 217.

### Intermediate 33: tert-butyl 4-[1-(4-amino-3-hydroxybut-1-yn-1-yl)cyclopropyl]piperazine-1-carboxylate

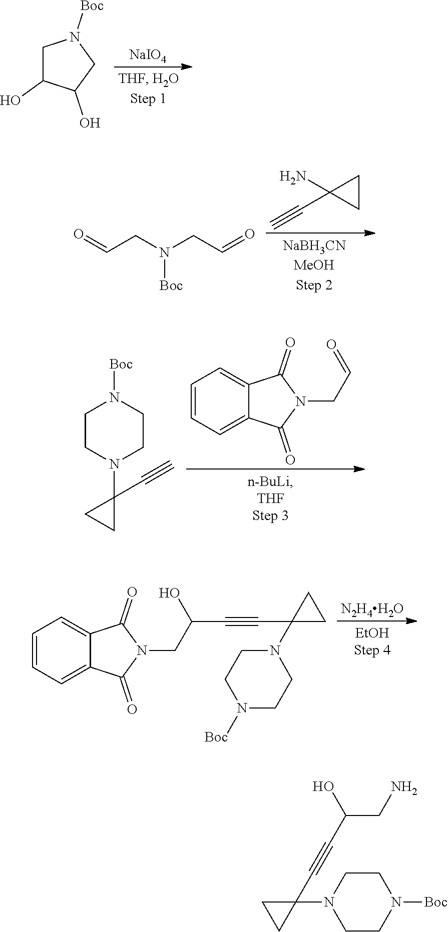

Step 1: A solution of tert-butyl 3,4-dihydroxypyrrolidine-1-carboxylate (10 g, 49.2 mmol) and NaIO₄ (21 g, 98.4 mmol) in THF (600 mL) was stirred at room temperature overnight. The reaction progress was monitored by TLC. Desired product was detected by TLC (PE/EA=3:1, Rf=0.5). The reaction was quenched by the addition of water (300 mL) at room temperature. The resulting mixture was extracted with EtOAc (1000 mL). The combined organic layers were washed with brine (300 mL), dried over anhydrous MgSO₄. After filtration, the filtrate was concentrated under reduced pressure. This resulted in tert-butyl N, N-bis(2-oxoethyl)carbamate (9.07 g) as a brown oil. The crude product was used in the next step directly without further purification. LCMS (ESI) m/z: 202 [M+H]⁺.

Step 2: A solution of 1-ethynylcyclopropan-1-amine hydrochloride (5.68 g, 48.3 mmol) in methanol (400 mL) was treated with tert-butyl N,N-bis(2-oxoethyl)carbamate (9.72 g, 48.31 mmol) at room temperature for 30 min under nitrogen atmosphere followed by the addition of NaBH₃CN (9.11 g, 144.9 mmol) in portions at 0° C. The resulting mixture was stirred at room temperature overnight under nitrogen atmosphere. The reaction was quenched by the addition of water (200 mL) at room temperature. The resulting mixture was extracted with EtOAc (500 mL). The combined organic layers were washed with brine (300 mL), dried over anhydrous MgSO₄. After filtration, the filtrate was concentrated under reduced pressure. The reaction progress was monitored by TLC. Desired product was detected by TLC (PE/EA=3:1, Rf=0.5). The residue was purified by silica gel column chromatography, eluted with PE/EA (3:1) to afford tert-butyl 4-(1-ethynylcyclopropyl)piperazine-1-carboxylate (4.8 g) as a white solid. LCMS (ESI) m/z: 251 [M+H]⁺.

Step 3: In to a solution of tert-butyl 4-(1-ethynylcyclopropyl)piperazine-1-carboxylate (2.3 g, 9.19 mmol) in THF (200 mL) was added dropwise n-Butyllithium (2.5 M in n-hexane, 4.41 mL, 11.02 mmol) at −78° C. under N₂ atmosphere. The reaction mixture was stirred at −78° C. for 30 mins. Then a solution of 2-(1,3-dioxoisoindolin-2-yl)acetaldehyde (2.09 g, 11 mmol) in THF (20 mL) was added dropwise and the mixture was stirred for another 2 h. The reaction was quenched by the addition of water (15 mL) at room temperature. The resulting mixture was concentrated under reduced pressure. The residue was purified by silica gel column chromatography, eluted with CH₂Cl₂/MeOH (9:1) to afford tert-butyl 4-{1-[4-(1,3-dioxoisoindol-2-yl)-3-hydroxybut-1-yn-1-yl]cyclopropyl}piperazine-1-carboxylate (1.5 g) as a brown oil. LCMS (ESI) m/z: 440 [M+H]⁺.

Step 4: A solution of tert-butyl 4-{1-[4-(1,3-dioxoisoindol-2-yl)-3-hydroxybut-1-yn-1-yl]cyclopropyl}piperazine-1-carboxylate (1.5 g, 3.41 mmol) in methylamine (3.1-3.5 M in THF, 20 mL, 50 mmol) was stirred for 1 min at room temperature. The reaction mixture was stirred at 70° C. for 16 h. The mixture was allowed to cool down to room temperature. The resulting mixture was filtered, the filter cake was washed EtOAc (3×50 mL), dried over anhydrous MgSO₄. The filtrate was concentrated under reduced pressure. This resulted in tert-butyl 4-[1-(4-amino-3-hydroxybut-1-yn-1-yl)cyclopropyl]piperazine-1-carboxylate (0.85 g) as a brown oil, which was used in the next step directly without further purification. LCMS (ESI) m/z: 310 [M+H]⁺.

Intermediate 33 was used for the synthesis of Example 229.

### Intermediate 34: (3-cyclopropyl-1-methylpyrazol-4-yl)methanol

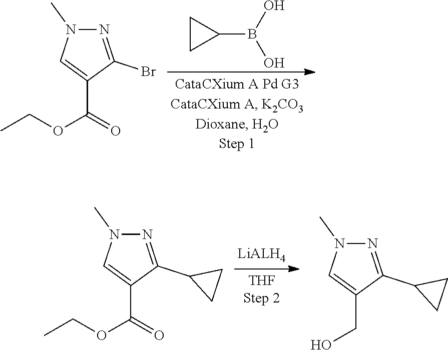

Step 1: A mixture of ethyl 3-bromo-1-methylpyrazole-4-carboxylate (2 g, 8.58 mmol), cyclopropylboronic acid (1.11 g, 12.9 mmol), CataCXium A Pd G3 (625 mg, 0.86 mmol), di(1-adamantyl)-N-butylphosphine (615.4 mg, 1.72 mmol), K₂CO₃ (3.56 g, 25.7 mmol), 1,4-dioxane (5 mL) in H₂O (0.5 mL) was stirred 2 h at 100° C. under nitrogen atmosphere. The mixture was allowed to cool down to room temperature. The residue was dissolved in H₂O (50 mL) and extracted with EtOAc (3×50 mL). The combined organic layers were washed with H₂O (3×50 mL), dried over anhydrous Na₂SO₄. After filtration, the filtrate was concentrated under reduced pressure. The residue was purified by silica gel column chromatography, eluted with PE/EA (8:1) to afford ethyl 3-cyclopropyl-1-methylpyrazole-4-carboxylate (1.3 g) as a yellow solid. LCMS (ESI) m/z: 195.1 [M+H]⁺.

Step 2: A mixture of ethyl 3-cyclopropyl-1-methylpyrazole-4-carboxylate (1.3 g, 6.69 mmol), LAH (508 mg, 13.4 mmol) in THF (30 mL) was stirred for 2 h at room temperature under nitrogen atmosphere. The reaction was quenched by the addition of sodium sulfate decahydrate (50 mg) at 0° C. The resulting mixture was filtered, the filter cake was washed with THF (3×50 mL). The filtrate was concentrated under reduced pressure. The residue was purified by silica gel column chromatography, eluted with CH₂Cl₂/MeOH (10:1) to afford (3-cyclopropyl-1-methylpyrazol-4-yl)methanol (800 mg) as a colorless oil. LCMS (ESI) m/z: 153.1 [M+H]⁺.

Intermediate 34 was used for the synthesis of Example 230.

### Intermediate 35: (4R)-4-aminopentan-2-ol hydrochloride

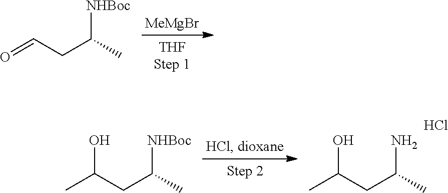

Step 1: To a stirred solution of methylmagnesium bromide (3M in 2-MeTHF, 3.56 mL, 5.34 mmol) in THF (20 mL) was added tert-butyl N-[(2R)-4-oxobutan-2-yl]carbamate (1 g, 5.34 mmol) at −78° C. under nitrogen atmosphere. The resulting mixture was stirred at room temperature for 2 h under nitrogen atmosphere. The reaction was quenched by the addition of sat. NH₄Cl (aq.) (100 mL) at room temperature. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with EtOAc/PE (10%-30%) to afford tert-butyl N-[(2R)-4-hydroxypentan-2-yl]carbamate (400 mg) as a yellow oil. LCMS (ESI) m/z: 204.0 [M+H]⁺.

Step 2: A solution of tert-butyl N-[(2R)-4-hydroxypentan-2-yl]carbamate (400 mg, 1.96 mmol) in HCl in 1,4-dioxane (4.0 M, 10 mL) was stirred at room temperature for 2 h. The resulting mixture was dried under vacuum to afford the desired product (150 mg) as a brown oil. LCMS (ESI) m/z: 104.0 [M+H]⁺.

Intermediate 35 was used for the synthesis of Examples 235 and 236.

### Intermediate 36: 3-bromo-4-(bromomethyl)-1-methylpyrazole

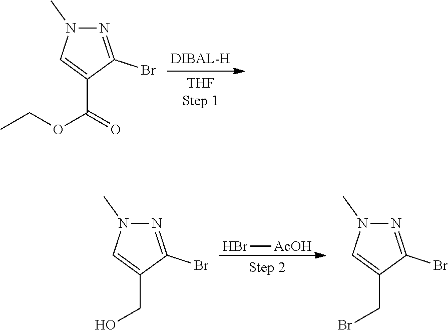

Step 1: A mixture of ethyl 3-bromo-1-methylpyrazole-4-carboxylate (2 g, 8.58 mmol), diisobutylaluminium hydride (25% in toluene, 2.44 g, 17.2 mmol) in THF (10 mL) was stirred for 10 h at room temperature under nitrogen atmosphere. The reaction was quenched with sodium sulfate decahydrate at 0° C. The resulting mixture was filtered, the filter cake was washed with THF (3×10 mL). The filtrate was concentrated under reduced pressure. The residue was purified by silica gel column chromatography, eluted with CH₂Cl₂/MeOH (7:1) to afford (3-bromo-1-methylpyrazol-4-yl)methanol (1.2 g, 73%) as a colorless oil. LCMS (ESI) m/z: 191, 193 [M+H]⁺.

Step 2: A mixture of (3-bromo-1-methylpyrazol-4-yl)methanol (500 mg, 2.62 mmol) and hydrogen bromide-acetic acid solution (10 mL) was stirred overnight at 100° C. The solvents were removed, precipitated solids were collected by filtration and washed with diethyl ether (3×10 mL). The resulting solid was dried under vacuum. This resulted in 3-bromo-4-(bromomethyl)-1-methylpyrazole (500 mg) as a brown solid. LCMS (ESI) m/z: 253, 255 [M+H]

Intermediate 36 was used for the synthesis of Example 243.

### Intermediate 37: 4-fluoro-5-iodo-1-methylpyrazole

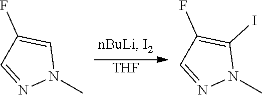

To a solution of 4-fluoro-1-methylpyrazole (1 g, 9.99 mmol) in THF (10 mL) was added dropwise n-butyllithium solution (2.5 M in THF/hexane, 4.8 mL) at −78° C. under N₂ atmosphere. The reaction mixture was stirred at −78° C. for 30 min. Then a solution of iodine (2.79 g, 11 mmol) in THF (3 mL) was added dropwise and the mixture was stirred for another 1 h. The reaction was quenched with water/sat. NH₄Cl (10 mL), and then the mixture was extracted with EtOAc (2×15 mL). The combined organic extracts were washed with brine (10 mL), dried over anhydrous Na₂SO₄.The residue was purified by silica gel column chromatography, eluted with PE/EA (12:1) to afford 4-fluoro-5-iodo-1-methylpyrazole (1 g) as a yellow solid. LCMS (ESI) m/z: 227 [M+H]⁺.

Intermediate 37 was used for the synthesis of Example 245.

### Intermediate 38: tert-butyl (2R)-2-(4-amino-3-hydroxybut-1-yn-1-yl)pyrrolidine-1-carboxylate

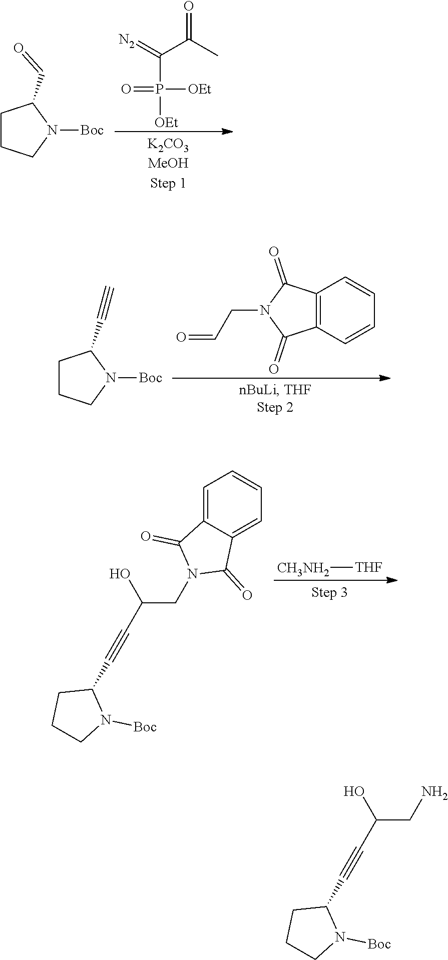

Step 1: To a stirred mixture of tert-butyl (2R)-2-formylpyrrolidine-1-carboxylate (5 g, 25.1 mmol) and (1-diazo-2-oxopropyl)-phosphonic acid dimethyl ester (7.23 g, 37.6 mmol) in methanol (20 mL) was added K₂CO₃ (6.94 g, 50.2 mmol) in portions at 0° C. The resulting mixture was stirred overnight at room temperature. The reaction was quenched by the addition of sat. NH₄Cl (aq.) (100 mL) at room temperature. The resulting mixture was extracted with EtOAc (3×50 mL). The combined organic layers were washed with H₂O (3×50 mL), dried over anhydrous Na₂SO₄. After filtration, the filtrate was concentrated under reduced pressure. The residue was purified by silica gel column chromatography, eluted with PE/EA (5:1) to afford tert-butyl (2R)-2-ethynylpyrrolidine-1-carboxylate as a colorless oil. LCMS (ESI) m/z: 196.1 [M+H]⁺.

Step 2: To a stirred solution of tert-butyl (2R)-2-ethynylpyrrolidine-1-carboxylate (2 g, 10.2 mmol) in THF (50 mL) was added butyllithium (1.6 M in n-hexane, 1.38 g, 21.5 mmol) dropwise at −78° C. under nitrogen atmosphere. The resulting mixture was stirred at −78° C. for 1 h under nitrogen atmosphere. To the above mixture was added 2-(1,3-dioxoisoindolin-2-yl)acetaldehyde (1.94 g, 10.2 mmol) dropwise over 3 min at −78° C. The resulting mixture was stirred at room temperature for additional 2 h. The reaction was quenched with MeOH (10 mL) at room temperature and concentrated under vacuum. The residue was purified by silica gel column chromatography, eluted with PE/EA (6:1) to afford tert-butyl (2R)-2-[4-(1,3-dioxoisoindol-2-yl)-3-hydroxybut-1-yn-1-yl]pyrrolidine-1-carboxylate (2 g) as a yellow oil. LCMS (ESI) m/z: 385.2 [M+H]⁺.

Step 3: A mixture of tert-butyl (2R)-2-[4-(1,3-dioxoisoindol-2-yl)-3-hydroxybut-1-yn-1-yl]pyrrolidine-1-carboxylate (2 g, 5.2 mmol) in methylamine (3.1-3.5 M in TIFF, 50 mL) was stirred overnight at 70° C. under nitrogen atmosphere. The mixture was allowed to cool down to room temperature. The resulting mixture was filtered, the filter cake was washed with THF (3×10 mL). The filtrate was concentrated under reduced pressure. This resulted in tert-butyl (2R)-2-(4-amino-3-hydroxybut-1-yn-1-yl)pyrrolidine-1-carboxylate as a brown solid. LCMS (ESI) m/z: 255.2 [M+H]⁺.

Intermediate 38 was used for the synthesis of Example 248.

### I. Biological Evaluation

### Example 1: Nuclear PAR Accumulation Assay

Compound IC50 values for inhibition of cellular PARG were determined using an immunofluorescence high-content imaging assay for nuclear PAR accumulation. HeLa cells were plated at 15,000 cells/well in a 96-well plate the day before compound treatment and then treated with varying concentrations of test compound and incubated for 1 hour at 37° C.+5% CO₂. Following test compound treatment, methylmethane sulfonate (MMS) was added to a final concentration of 50 μg/mL and the cells were incubated for an additional hour. Following treatment, the cells were fixed with ice cold 95% methanol and the plates sealed and placed at -20° C. for 15 minutes. The cells were then washed once with phosphate buffered saline (PBS) and permeabilized with 0.10% Triton-X 100 for 20 minutes at room temperature. The cells were again washed once with PBS and incubated with an anti-poly-ADP ribose (PAR) monoclonal antibody (Millipore Part #AM80 clone 1OH) diluted 1:1,000 in 5% fetal bovine serum+0.1% Tween-20+PBS overnight at 4° C. The cells were then washed three times with PBS before incubation with a fluorescently conjugated goat anti-mouse antibody (Invitrogen A32723) diluted 1:500 in 5% FBS+0.1% Tween-20+PBS for 1 hour at room temperature. The cells were again washed three times with PBS and the nuclei labeled by incubating with 1 μg/mL DAPI (4′,6-diamidino-2-phenylindole). Nuclear PAR accumulation was evaluated using high-content fluorescence microscopy. Four fields for each well were acquired at 20× magnification resulting in at least 1,000 cells analyzed per well. Using high-content software (CellReporterXpress) the nuclei were detected and segmented using the DAPI channel and the fluorescence intensity of the PAR-Alexa 488 channel was quantified within the nuclei regions. Both the average intensity per nucleus and percent PAR positive cells were quantified. Percent PAR positive cells were identified by having greater anti-PAR labeling above the threshold set with vehicle DMSO treated control cells. Percent inhibition of PARG by test compounds was calculated by normalizing the data to the 0.1% DMSO and reference compound control values, which represent 0% and 100% inhibition of enzymatic activity, respectively. Concentration-response-curves and IC50 values were generated using four parameter logistic curve fit software.

Representative data for exemplary compounds is presented in Table 32, Table 33 and Table 34.

TABLE 32

| | PAR | | PAR | | PAR |
|---|---|---|---|---|---|
| | Accumulation | | Accumulation | | Accumulation |
| Example | IC₅₀ (μM) | Example | IC₅₀ (μM) | Example | IC₅₀ (μM) |
| 1 | A | 30 | A | 59 | B |
| 2 | A | 31 | A | 60 | B |
| 3 | B | 32 | A | 61 | B |
| 4 | A | 33 | A | 62 | C |
| 5 | A | 34 | A | 63 | C |
| 6 | A | 35 | A | 64 | C |
| 7 | B | 36 | A | 65 | A |
| 8 | A | 37 | B | 66 | A |
| 9 | B | 38 | A | 67 | A |
| 10 | A | 39 | A | 68 | A |
| 11 | A | 40 | A | 69 | B |
| 12 | A | 41 | B | 70 | C |
| 13 | B | 42 | A | 71 | B |
| 14 | A | 43 | A | 72 | A |
| 15 | A | 44 | B | 73 | B |
| 16 | A | 45 | A | 74 | C |
| 17 | A | 46 | A | 75 | B |
| 18 | B | 47 | A | 76 | A |
| 19 | A | 48 | A | 77 | A |
| 20 | A | 49 | A | 78 | B |
| 21 | B | 50 | B | 79 | B |
| 22 | B | 51 | A | 80 | B |
| 23 | A | 52 | A | 81 | A |
| 24 | A | 53 | C | 82 | C |
| 25 | A | 54 | B | 83 | A |
| 26 | B | 55 | A | 84 | B |
| 27 | B | 56 | A | 85 | A |
| 28 | A | 57 | B | 86 | A |
| 29 | A | 58 | B | 87 | A |
| 88 | A | 91 | B | 94 | B |
| 89 | C | 92 | B | 95 | B |
| 90 | B | 93 | B | — | — |

TABLE 33

| | PAR | | PAR | | PAR |
|---|---|---|---|---|---|
| | Accumulation | | Accumulation | | Accumulation |
| Example | IC₅₀ (μM) | Example | IC₅₀ (μM) | Example | IC₅₀ (μM) |
| 96 | A | 125 | A | 154 | A |
| 97 | A | 126 | B | 155 | A |
| 98 | A | 127 | A | 156 | B |
| 99 | A | 128 | B | 157 | A |
| 100 | A | 129 | A | 158 | B |
| 101 | A | 130 | A | 159 | C |
| 102 | A | 131 | C | 160 | B |
| 103 | B | 132 | B | 161 | C |
| 104 | A | 133 | A | 162 | A |
| 105 | B | 134 | A | 163 | A |
| 106 | B | 135 | B | 164 | A |
| 107 | A | 136 | A | 165 | A |
| 108 | A | 137 | B | 166 | A |
| 109 | B | 138 | B | 167 | A |
| 110 | A | 139 | B | 168 | B |
| 111 | A | 140 | A | 169 | B |
| 112 | A | 141 | B | 170 | A |
| 113 | A | 142 | A | 171 | B |
| 114 | B | 143 | B | 172 | A |
| 115 | A | 144 | A | 173 | A |
| 116 | A | 145 | A | 174 | A |
| 117 | A | 146 | C | 175 | A |
| 118 | A | 147 | A | 176 | B |
| 119 | B | 148 | A | 177 | B |
| 120 | A | 149 | A | 178 | B |
| 121 | A | 150 | A | 179 | A |
| 122 | A | 151 | A | 180 | A |
| 123 | A | 152 | A | 181 | A |
| 124 | A | 153 | A | 182 | A |
| 183 | A | 210 | A | 237 | C |
| 184 | A | 211 | A | 238 | A |
| 185 | A | 212 | A | 239 | B |
| 186 | A | 213 | B | 240 | B |
| 187 | A | 214 | C | 241 | A |
| 188 | A | 215 | A | 242 | A |
| 189 | A | 216 | A | 243 | A |
| 190 | A | 217 | A | 244 | A |
| 191 | A | 218 | A | 245 | A |
| 192 | A | 219 | A | 246 | A |
| 193 | A | 220 | B | 247 | B |
| 194 | A | 221 | A | 248 | B |
| 195 | A | 222 | A | 249 | A |
| 196 | A | 223 | B | 250 | A |
| 197 | A | 224 | A | 251 | A |
| 198 | A | 225 | A | 252 | A |
| 199 | A | 226 | B | 253 | A |
| 200 | B | 227 | A | 254 | B |
| 201 | A | 228 | A | 255 | A |
| 202 | A | 229 | B | 256 | A |
| 203 | A | 230 | A | 257 | A |
| 204 | A | 231 | A | 258 | A |
| 205 | B | 232 | A | 259 | A |
| 206 | A | 233 | A | 260 | A |
| 207 | A | 234 | A | 261 | A |
| 208 | A | 235 | B | 262 | A |
| 209 | A | 236 | A | 263 | A |

TABLE 34

| | PAR |
|---|---|
| | Accumulation |
| Example | IC₅₀ (μM) |
| 264 | A |
| 265 | A |
| 266 | A |
| 267 | C |
| 268 | A |
| 269 | A |
| 270 | A |
| 271 | A |
| 272 | A |
| 273 | B |
| 274 | A |
| 275 | A |
| 276 | A |
| 277 | A |
| 278 | B |
| 279 | B |
| 280 | A |
| 281 | A |
| 282 | A |
| 283 | C |
| 284 | C |
| 285 | B |
| 286 | A |
| 287 | A |
| 288 | A |
| 289 | A |
| 290 | B |
| 291 | B |
| 292 | B |
| 293 | B |
| 294 | A |
| 295 | B |
| 296 | B |
| 297 | A |

Note:
PAR accumulation assay IC₅₀ data are designated within the following ranges:
A: ≤0.1 μM
B: >0.1 μM to ≤1.0 μM
C: >1.0 μM to ≤10 μM
D: >10 μM

### III. Preparation of Pharmaceutical Dosage Forms

### Example 1: Oral Capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Table 1, or a pharmaceutically acceptable salt or solvate thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

What is claimed is:

1. A compound, or a pharmaceutically acceptable salt, stereoisomer, or deuteroisotope thereof, having the structure of Formula (I): 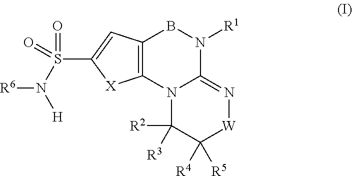
wherein,
B is a bond, C═O, or CN—OR⁹;
W is a bond, —O—, or C(R¹⁰)(R¹¹), or NR⁷;
X is —S—, or —*Y═C(A)—; wherein the * indicates point of attachment to C—SO₂ group;
Y is N, C—H, C—F, C—O(optionally substituted C1-C6 alkyl), C—NH₂, C—NH(optionally substituted C1-C6 alkyl), C—N(optionally substituted C1-C6 alkyl)₂, C—SH, C—S(optionally substituted C1-C6 alkyl), C—NH(optionally substituted (heteroaryl)alkylene), —C—NH(optionally substituted (heteroaryl)alkynylene);
A is selected from the group consisting of hydrogen, halo, —OH, —CN, optionally substituted C1-C6 alkoxy, —N(R⁷)₂, optionally substituted C1-C6 alkyl, optionally substituted C2-C6 alkenyl, optionally substituted C2-C6 alkynyl, optionally substituted C3-C7 carbocyclyl, optionally substituted (carbocyclyl)alkylene, optionally substituted (carbocyclyl)alkynylene, optionally substituted heterocyclyl, optionally substituted (heterocyclyl)alkylene, optionally substituted heteroaryl, and optionally substituted aryl;
R¹ is selected from the group consisting of optionally substituted alkyl, optionally substituted alkenyl, optionally substituted alkynyl, optionally substituted (cycloalkyl)alkylene, optionally substituted (aryl)alkylene, optionally substituted (heterocyclyl)alkylene, optionally substituted (heteroaryl)alkylene, optionally substituted (heteroaryl)alkynylene, optionally substituted aryl, optionally substituted heteroaryl, optionally substituted C3-C7 carbocyclyl, and optionally substituted heterocyclyl;
R² and R³ are independently selected from the group consisting of hydrogen, halo, —OH, —CN, optionally substituted alkyl, optionally substituted alkenyl, optionally substituted alkynyl, optionally substituted cycloalkyl, optionally substituted aryl, optionally substituted heteroaryl, optionally substituted heterocyclyl, optionally substituted (cycloalkyl)alkylene, optionally substituted (heterocyclyl)alkylene, optionally substituted (aryl)alkylene, optionally substituted (heteroaryl)alkylene, optionally substituted (cycloalkyl)alkenylene, optionally substituted (heterocyclyl)alkenylene, optionally substituted (aryl)alkenylene, optionally substituted (heteroaryl)alkenylene, optionally substituted (cycloalkyl)alkynylene, optionally substituted (heterocyclyl)alkynylene, optionally substituted (heteroaryl)alkynylene, optionally substituted (aryl)alkynylene, optionally substituted (cycloalkyl)-O-alkylene, optionally substituted (heterocyclyl)-O-alkylene, optionally substituted (aryl)-O-alkylene, optionally substituted (heteroaryl)-O-alkylene, optionally substituted (cycloalkyl)-NR⁷-alkylene, optionally substituted (heterocyclyl)-NR⁷-alkylene, optionally substituted (aryl)-NR⁷-alkylene, optionally substituted (heteroaryl)-NR⁷-alkylene, optionally substituted (alkyl)-O-alkylene, optionally substituted (alkyl)-NR⁷-alkylene, optionally substituted (alkyl)-O-alkynylene, and optionally substituted (alkyl)-NR⁷-alkynylene; or
optionally, R² and R³ combine to form an optionally substituted carbocyclic or heterocyclic ring;
R⁴, R⁵, R¹⁰, and R¹¹ are independently selected from the group consisting of hydrogen, halo, —OH, —CN, optionally substituted alkyl, optionally substituted alkenyl, optionally substituted alkynyl, optionally substituted cycloalkyl, optionally substituted (cycloalkyl)alkylene, optionally substituted (cycloalkyl)alkynylene, optionally substituted heterocyclyl, optionally substituted (heterocyclyl)alkylene, optionally substituted heteroaryl, and optionally substituted aryl;
or optionally, R⁴ and R⁵ combine to form an optionally substituted carbocyclic or heterocyclic ring;
or optionally, R³ and R⁴ combine to form an optionally substituted carbocyclic or heterocyclic ring;
R⁶ is optionally substituted alkyl, optionally substituted cycloalkyl, optionally substituted heterocyclyl, or an optionally substituted 1,1′-bi(cyclopropan)-1-yl;
each R⁷ is independently hydrogen or an optionally substituted C1-C6 alkyl; and
each R⁹ is independently hydrogen or an optionally substituted C1-C6 alkyl.

2. The compound of claim 1, or a pharmaceutically acceptable salt, stereoisomer, or deuteroisotope thereof, wherein X is -S-.

3. The compound of claim 1, or a pharmaceutically acceptable salt, stereoisomer, or deuteroisotope thereof, having the structure of Formula (II):

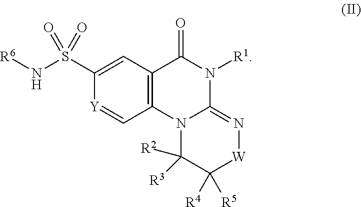

4. The compound of claim 3, or a pharmaceutically acceptable salt, stereoisomer, or deuteroisotope thereof, wherein W is a bond.

5. The compound of claim 3, or a pharmaceutically acceptable salt, stereoisomer, or deuteroisotope thereof, wherein W is O.

6. The compound of claim 3, or a pharmaceutically acceptable salt, stereoisomer, or deuteroisotope thereof, wherein W is CR¹⁰R¹¹.

7. The compound of claim 1, or a pharmaceutically acceptable salt, stereoisomer, or deuteroisotope thereof, having the structure of Formula (III):

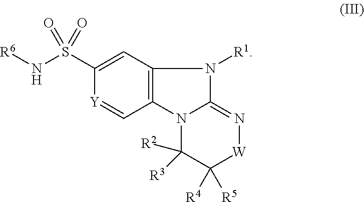

8. The compound of claim 7, or a pharmaceutically acceptable salt, stereoisomer, or deuteroisotope thereof, wherein W is a bond.

9. The compound of claim 7, or a pharmaceutically acceptable salt, stereoisomer, or deuteroisotope thereof, wherein W is O.

10. The compound of claim 7, or a pharmaceutically acceptable salt, stereoisomer, or deuteroisotope thereof, wherein W is CR¹⁰R¹¹.

11. The compound of any one of claims 1, or 3-10, or a pharmaceutically acceptable salt, stereoisomer, or deuteroisotope thereof, wherein Y is N.

12. The compound of any one of claims 1, or 3-10, or a pharmaceutically acceptable salt, stereoisomer, or deuteroisotope thereof, wherein Y is C—H.

13. The compound of any one of claims 1, or 3-10, or a pharmaceutically acceptable salt, stereoisomer, or deuteroisotope thereof, wherein Y is C—F.

14. The compound of any one of claims 1, or 3-13, or a pharmaceutically acceptable salt, stereoisomer, or deuteroisotope thereof, wherein A is hydrogen.

15. The compound of any one of claims 1, or 3-13, or a pharmaceutically acceptable salt, stereoisomer, or deuteroisotope thereof, wherein A is halo or —CN.

16. The compound of any one of claims 1, or 3-13, or a pharmaceutically acceptable salt, stereoisomer, or deuteroisotope thereof, wherein A is optionally substituted C1-C6 alkoxy or -N(R⁷)₂.

17. The compound of any one of claims 1, or 3-13, or a pharmaceutically acceptable salt, stereoisomer, or deuteroisotope thereof, wherein A is selected from the group consisting optionally substituted C1-C6 alkyl, optionally substituted C3-C7 carbocyclyl, and optionally substituted (carbocyclyl)alkylene.

18. The compound of any one of claims 1, or 3-13, or a pharmaceutically acceptable salt, stereoisomer, or deuteroisotope thereof, wherein A is optionally substituted heterocyclyl or optionally substituted (heterocyclyl)alkylene.

19. The compound of any one of claims 1, or 3-13, or a pharmaceutically acceptable salt, stereoisomer, or deuteroisotope thereof, wherein A is selected from optionally substituted heteroaryl or optionally substituted aryl.

20. The compound of any one of claims 1-19, or a pharmaceutically acceptable salt, stereoisomer, or deuteroisotope thereof, wherein R¹ is optionally substituted alkyl.

21. The compound of claim 20, or a pharmaceutically acceptable salt, stereoisomer, or deuteroisotope thereof, wherein R¹ is optionally substituted C1-C4 alkyl.

22. The compound of claim 20, or a pharmaceutically acceptable salt, stereoisomer, or deuteroisotope thereof, wherein R¹ is optionally substituted C1-C4 alkyl and is substituted with at least one substituent selected from —CN, —OR⁹, halo, oxo, —N(R⁹)₂, or —CON(R⁹)₂; wherein each R⁹ is independently hydrogen, or optionally substituted C1-C4 alkyl.

23. The compound of any one of claims 1-19, or a pharmaceutically acceptable salt, stereoisomer, or deuteroisotope thereof, wherein R¹ is optionally substituted (heteroaryl)alkylene.

24. The compound of claim 23, or a pharmaceutically acceptable salt, stereoisomer, or deuteroisotope thereof, wherein the optionally substituted heteroaryl is selected from a 5- or a 6-membered heteroaryl.

25. The compound of claim 24, or a pharmaceutically acceptable salt, stereoisomer, or deuteroisotope thereof, wherein the heteroaryl is selected from a 5-membered nitrogen-containing heteroaryl.

26. The compound of claim 25, or a pharmaceutically acceptable salt, stereoisomer, or deuteroisotope thereof, wherein the optionally substituted 5-membered nitrogen-containing heteroaryl is selected from optionally substituted thiazole, optionally substituted oxazole, optionally substituted imidazole, optionally substituted pyrazole, optionally substituted isoxazole, optionally substituted pyrrole, optionally substituted oxadiazole, optionally substituted triazole, optionally substituted thiadiazole, or optionally substituted isothiazole or optionally substituted isoxazole.

27. The compound of claim 25, or a pharmaceutically acceptable salt, stereoisomer, or deuteroisotope thereof, wherein the optionally substituted 5-membered nitrogen-containing heteroaryl is selected from optionally substituted pyrazole or optionally substituted isoxazole.

28. The compound of claim 24, or a pharmaceutically acceptable salt, stereoisomer, or deuteroisotope thereof, wherein the heteroaryl is a 6-membered nitrogen-containing heteroaryl.

29. The compound of claim 28, or a pharmaceutically acceptable salt, stereoisomer, or deuteroisotope thereof, wherein the 6-membered nitrogen-containing heteroaryl is an optionally substituted pyridine or optionally substituted pyrazine.

30. The compound of any one of claims 23-29, or a pharmaceutically acceptable salt, stereoisomer, or deuteroisotope thereof, wherein R¹ is optionally substituted heteroaralkyl and the alkylene is an optionally substituted C1-C4 alkylene.

31. The compound of claim 30, or a pharmaceutically acceptable salt, stereoisomer, or deuteroisotope thereof, wherein the optionally substituted C1-C4 alkylene is a —CH₂-.

32. The compound of claim 30, or a pharmaceutically acceptable salt, stereoisomer, or deuteroisotope thereof, wherein the optionally substituted C1-C4 alkylene is a —CD₂-.

33. The compound of any one of claims 1-19, or a pharmaceutically acceptable salt, stereoisomer, or deuteroisotope thereof, wherein R¹ is optionally substituted (aryl)alkylene.

34. The compound of claim 33, or a pharmaceutically acceptable salt, stereoisomer, or deuteroisotope thereof, wherein the optionally substituted (aryl)alkylene comprises an optionally substituted phenyl.

35. The compound of claim 33 or 34, or a pharmaceutically acceptable salt, stereoisomer, or deuteroisotope thereof, wherein the optionally substituted (aryl)alkylene comprises an optionally substituted C1-C4 alkylene.

36. The compound of claim 35, or a pharmaceutically acceptable salt, stereoisomer, or deuteroisotope thereof, wherein the optionally substituted C1-C4 alkylene is a —CH₂— or —CD₂-.

37. The compound of any one of claims 1-19, or a pharmaceutically acceptable salt, stereoisomer, or deuteroisotope thereof, wherein R¹ is an optionally substituted C4-C7 (carbocyclyl)alkylene.

38. The compound of claim 37, or a pharmaceutically acceptable salt, stereoisomer, or deuteroisotope thereof, wherein the optionally substituted (carbocyclyl)alkylene is an optionally substituted (cyclopropyl)methylene.

39. The compound of any one of claims 1-19, or a pharmaceutically acceptable salt, stereoisomer, or deuteroisotope thereof, wherein R¹ is an optionally substituted (heterocyclyl)alkylene.

40. The compound of any one of claims 1-19, or a pharmaceutically acceptable salt, stereoisomer, or deuteroisotope thereof, wherein R¹ is

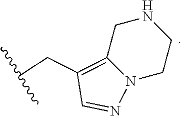 [SMILES: *Cc1cnn2c1CNCC2]

41. The compound of any one of claims 1-40, or a pharmaceutically acceptable salt, stereoisomer, or deuteroisotope thereof, wherein R⁴ is hydrogen or methyl.

42. The compound of any one of claims 1-41, or a pharmaceutically acceptable salt, stereoisomer, or deuteroisotope thereof, wherein R⁵ is hydrogen or methyl.

43. The compound of any one of claims 1-42, or a pharmaceutically acceptable salt, stereoisomer, or deuteroisotope thereof, wherein R³ is hydrogen or methyl.

44. The compound of any one of claims 1-43, or a pharmaceutically acceptable salt, stereoisomer, or deuteroisotope thereof, wherein R² is hydrogen.

45. The compound of any one of claims 1-43, or a pharmaceutically acceptable salt, stereoisomer, or deuteroisotope thereof, wherein R² is not hydrogen.

46. The compound of claim 45, or a pharmaceutically acceptable salt, stereoisomer, or deuteroisotope thereof, wherein R² is an optionally substituted (heteroaryl)alkynylene.

47. The compound of claim 46, or a pharmaceutically acceptable salt, stereoisomer, or deuteroisotope thereof, wherein the optionally substituted (heteroaryl)alkynyl is selected from:

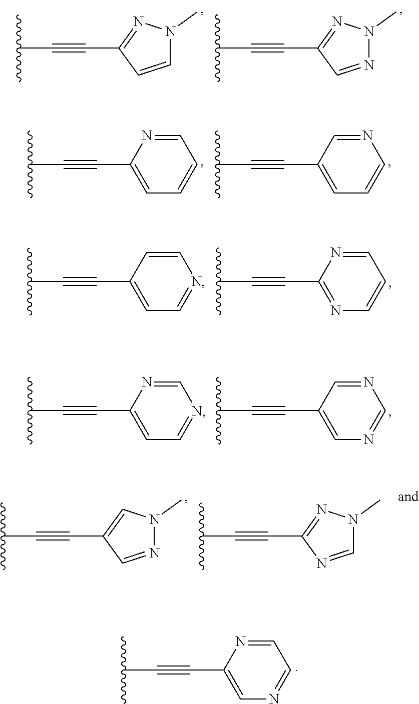

48. The compound of claim 45, or a pharmaceutically acceptable salt, stereoisomer, or deuteroisotope thereof, wherein R² is an optionally substituted (aryl)alkynylene.

49. The compound of claim 46, or a pharmaceutically acceptable salt, stereoisomer, or deuteroisotope thereof, wherein the optionally substituted (aryl)alkynylene is —C—C≡C6H₅.

50. The compound of claim 45, or a pharmaceutically acceptable salt, stereoisomer, or deuteroisotope thereof, wherein R² is an optionally substituted (aryl)alkenylene.

51. The compound of claim 48, or a pharmaceutically acceptable salt, stereoisomer, or deuteroisotope thereof, wherein the optionally substituted (aryl)alkenylene is —CH═CH—C6H₅.

52. The compound of claim 45, or a pharmaceutically acceptable salt, stereoisomer, or deuteroisotope thereof, wherein R² is an optionally substituted (aryloxy)alkylene.

53. The compound of claim 52, or a pharmaceutically acceptable salt, stereoisomer, or deuteroisotope thereof, wherein the optionally substituted (aryloxy)alkylene is 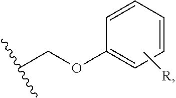
wherein R is selected from halo, —OH, —CN, optionally substituted C1-C6 alkoxy, or optionally substituted C1-C6 alkyl.

54. The compound of any one of claims 1-43, or a pharmaceutically acceptable salt, stereoisomer, or deuteroisotope thereof, wherein R² is optionally substituted alkynyl.

55. The compound of any one of claims 1-43, or a pharmaceutically acceptable salt, stereoisomer, or deuteroisotope thereof, wherein R² is optionally substituted C2-C4 alkynyl.

56. The compound of any one of claims 1-43, or a pharmaceutically acceptable salt, stereoisomer, or deuteroisotope thereof, wherein R² is —C≡C—H.

57. The compound of any one of claims 1-56, or a pharmaceutically acceptable salt, stereoisomer, or deuteroisotope thereof, wherein R⁶ is optionally substituted C3-C5 cycloalkyl.

58. The compound of claim 57, or a pharmaceutically acceptable salt, stereoisomer, or deuteroisotope thereof, wherein the optionally substituted cycloalkyl is: 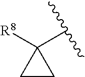
wherein R⁸ is selected from the group consisting of hydrogen, —CH₃, —CH₂F, —CHF₂, —CF₃, —CN, cyclopropyl, —CH₂CH₃, —CH(CH₃)₂, and —C(CH₃)3.

59. The compound of any one of claims 1 or 2, or a pharmaceutically acceptable salt, stereoisomer, or deuteroisotope thereof, wherein B is CN—OR⁹.

60. The compound of claim 59, or a pharmaceutically acceptable salt, stereoisomer, or deuteroisotope thereof, wherein R⁹ is H.

61. The compound of claim 59, or a pharmaceutically acceptable salt, stereoisomer, or deuteroisotope thereof, wherein R⁹ is optionally substituted C1-C3 alkyl.

62. A compound, or a pharmaceutically acceptable salt, stereoisomer, or deuteroisotope thereof, described in Table 1.

63. A compound, or a pharmaceutically acceptable salt, stereoisomer, or deuteroisotope thereof, as described in Table 2.

64. A pharmaceutical composition comprising a compound, or a pharmaceutically acceptable salt, stereoisomer, or deuteroisotope thereof, as described in any one of claims 1-63, and a pharmaceutically acceptable excipient.

65. A method of preparing a pharmaceutical composition comprising mixing a compound, or a pharmaceutically acceptable salt, stereoisomer, or deuteroisotope thereof, as described in any one of claims 1-63, and a pharmaceutically acceptable carrier.

66. A compound of any one of claims 1-63, or a pharmaceutically acceptable salt, stereoisomer, or deuteroisotope thereof, for use in a method of treatment of the human or animal body.

67. A method of treating cancer in a patient in need thereof, comprising administering to the patient a compound as described in any one of claims 1-63, or a pharmaceutically acceptable salt, stereoisomer, or deuteroisotope thereof.

68. A method of treating cancer in a patient in need thereof, comprising administering to the patient a pharmaceutical composition comprising a compound as described in any one of claims 1-63, or a pharmaceutically acceptable salt, stereoisomer, or deuteroisotope thereof, and a pharmaceutically acceptable excipient.

69. A method of inhibiting a PARG enzyme comprising contacting the enzyme with a compound of any one of claims 1-63, wherein the PARG enzyme is contacted in an in vitro setting.

70. A method of inhibiting a PARG enzyme comprising contacting the enzyme with a compound of any one of claims 1-63, wherein the PARG enzyme is contacted in an in vivo setting.
